SEQUENCE LISTING (781 sequences)

SEQ ID NO: 1 - protein, 574 aa, Respiratory Syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 574 aa, Respiratory Syncytial virus
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 3 - protein, 574 aa, Respiratory Syncytial virus
MELPILKTNAITTILAAVTLCFASSQNITEEFYQSTCSAVSKGYLSALRTGWYTSVITIELSNIKENKCNGTDAKVKLIKQELDKYKNAVTELQLLMQSTPAANSRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 4 - protein, 574 aa, Synthetic construct
MELPILKTNAITTILAAVTLCFASSQNITEEFYQSTCSAVSKGYLSALRTGWYTSVITIELSNIKENKCNGTDAKVKLIKQELDKYKNAVTELQLLMQSTPACNSRARRELPRFMNYTLNNTKNTNVTLSKKRKRRFLGFLLGVGSACASGIAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 5 - protein, 574 aa, Respiratory Syncytial virus
MELLIHRSSAIFLTLAINALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 6 - protein, 574 aa, Synthetic construct
MELLIHRSSAIFLTLAINALYLTSSQNITEEFYQSTCSAVSRGYFSALRTGWYTSVITIELSNIKETKCNGTDTKVKLIKQELDKYKNAVTELQLLMQNTPACNNRARREAPQYMNYTINTTKNLNVSISKKRKRRFLGFLLGVGSACASGIAVSKVLHLEGEVNKIKNALLSTNKAVVSLSNGVSVLTIKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSSEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 7 - DNA, 1728 bp, Respiratory Syncytial virus
  source 1..1728: mol_type = other DNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgccgccaatagcagagccagacgggaactgcctagattcatgaactacaccctgaacaacaccaagaacaccaacgtgaccctgagcaagaagcggaagcggagattcctgggctttctgctcggagtgggaagcgccattgcctctggaatcgccgtgtctaaagtgctgcacctggaaggcgaagtgaacaagatcaagagcgccctgctgagcaccaacaaggccgtggtgtctctgagcaatggcgtgtccgtgctgaccagcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcgacgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 8 - DNA, 1728 bp, Synthetic construct
  source 1..1728: mol_type = other DNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 9 - DNA, 1728 bp, Respiratory Syncytial virus
  source 1..1728: mol_type = other DNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgccgccaacaaccgggccagaagagaagcccctcagtacatgaactacaccatcaacaccaccaagaacctgaacgtgtccatcagcaagaagcggaagcggcggttcctgggctttctgcttggagtgggaagcgccattgccagcggaatcgccgtgtctaaagtgctgcacctggaaggcgaagtgaacaagatcaagaatgccctgctgagcaccaacaaggccgtggtgtctctgagcaatggcgtgtccgtgctgacctccaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcgacgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

SEQ ID NO: 10 - DNA, 1728 bp, Synthetic construct
  source 1..1728: mol_type = other DNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

SEQ ID NO: 11 - DNA, 2003 bp, Synthetic construct
  source 1..2003: mol_type = other DNA
aggaaataagagaggataagacgactaaggagacatacagaataagaaacaggcagccaccatggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 2003 bp, Synthetic construct
  source 1..2003: mol_type = other DNA
aggaaataagagaggataagacgactaaggagacatacagaataagaaacaggcagccaccatggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 1995 bp, Synthetic construct
  source 1..1995: mol_type = other DNA
aggtgtcagagtttaacttgaagactatttctagggataatatccctgccaccatggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 1995 bp, Synthetic construct
  source 1..1995: mol_type = other DNA
aggtgtcagagtttaacttgaagactatttctagggataatatccctgccaccatggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - RNA, 1728 nt, Synthetic construct
  source 1..1728: mol_type = other RNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 16 - RNA, 1728 nt, Synthetic construct
  source 1..1728: mol_type = other RNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

SEQ ID NO: 17 - RNA, 2003 nt, Synthetic construct
  source 1..2003: mol_type = other RNA
aggaaataagagaggataagacgactaaggagacatacagaataagaaacaggcagccaccatggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - RNA, 2003 nt, Synthetic construct
  source 1..2003: mol_type = other RNA
aggaaataagagaggataagacgactaaggagacatacagaataagaaacaggcagccaccatggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - RNA, 1995 nt, Synthetic construct
  source 1..1995: mol_type = other RNA
aggtgtcagagtttaacttgaagactatttctagggataatatccctgccaccatggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 20 - RNA, 1995 nt, Synthetic construct
  source 1..1995: mol_type = other RNA
aggtgtcagagtttaacttgaagactatttctagggataatatccctgccaccatggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 21 - DNA, 80 bp, Synthetic construct
  source 1..80: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 22 - RNA, 80 nt, Synthetic construct
  source 1..80: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 23 - DNA, 10 bp, Synthetic construct
  source 1..10: mol_type = other DNA
gcatatgact

SEQ ID NO: 24 - RNA, 10 nt, Synthetic construct
  source 1..10: mol_type = other RNA
gcatatgact

SEQ ID NO: 25-42 - skipped (18 entries)

SEQ ID NO: 43 - protein, 27 aa, T4 bacteriophage
GYIPEAPRDGQAYVRKDGEWVLLSTFL

SEQ ID NO: 44-48 - skipped (5 entries)

SEQ ID NO: 49 - DNA, 58 bp, Synthetic construct
  source 1..58: mol_type = other DNA
aggaaataagacgaggaatagagagtaagaagaaatataaggccagtgcgccgccacc

SEQ ID NO: 50 - DNA, 57 bp, Synthetic construct
  source 1..57: mol_type = other DNA
aggaaataagagaggataagacgactaaggagacatacagaataagaggcagccacc

SEQ ID NO: 51 - DNA, 59 bp, Synthetic construct
  source 1..59: mol_type = other DNA
aggaaataagaaagaagacagaagaagacagaagaagaaccagagaaggacaagccacc

SEQ ID NO: 52 - DNA, 50 bp, Synthetic construct
  source 1..50: mol_type = other DNA
agaagagaacctcgtcgagtcctggtagtagtaatcctagaggagccacc

SEQ ID NO: 53 - DNA, 50 bp, Synthetic construct
  source 1..50: mol_type = other DNA
aggatataaccaattttgtgaattacgagctaaaacgtcttcgtgccacc

SEQ ID NO: 54 - DNA, 50 bp, Synthetic construct
  source 1..50: mol_type = other DNA
aggagggtaattcgcttagcgatagtactatcgaagcgtacagagccacc

SEQ ID NO: 55 - DNA, 50 bp, Synthetic construct
  source 1..50: mol_type = other DNA
aggaggactgcgcgaacctgcatagtgatcataaggtcatgatagccacc

SEQ ID NO: 56 - DNA, 50 bp, Synthetic construct
  source 1..50: mol_type = other DNA
aggtatctaattcgacaattggtctacaaattatcctaacgcctgccacc

SEQ ID NO: 57 - DNA, 92 bp, Synthetic construct
  source 1..92: mol_type = other DNA
aggtccccataagatactttaacaagatcactaggtttagacgctgtgcagtcgtcccggactactacagccgcgccgcagtcttcgccacc

SEQ ID NO: 58 - DNA, 53 bp, Synthetic construct
  source 1..53: mol_type = other DNA
aggacatttgctcctgacacaaccgtgagcactagcaccttcacactgccacc

SEQ ID NO: 59 - DNA, 53 bp, Synthetic construct
  source 1..53: mol_type = other DNA
aggaaacttgctactaccagcaccatgctcgtcaacatcctcaactagccacc

SEQ ID NO: 60 - DNA, 53 bp, Synthetic construct
  source 1..53: mol_type = other DNA
aggacattagcttctgacacaactgccttcgctaccaacatcacaccgccacc

SEQ ID NO: 61 - DNA, 53 bp, Synthetic construct
  source 1..53: mol_type = other DNA
aggacatttgcttctgacaccactgtattcgctggcatcctcaacttgccacc

SEQ ID NO: 62 - DNA, 53 bp, Synthetic construct
  source 1..53: mol_type = other DNA
aggacatttacttctgacccaactgagttgactagcgacctgaactagccacc

SEQ ID NO: 63 - DNA, 53 bp, Synthetic construct
  source 1..53: mol_type = other DNA
aggacatttgcttctgtcacaactttgttcactagcagtatcgcccagccacc

SEQ ID NO: 64 - DNA, 53 bp, Synthetic construct
  source 1..53: mol_type = other DNA
aggacatttccttctgacacaactgtattcaaccgtaagctcaaaaagccacc

SEQ ID NO: 65 - DNA, 53 bp, Synthetic construct
  source 1..53: mol_type = other DNA
aggacattagtttctgacacaactgtcaggactagcaacatcaaccagccacc

SEQ ID NO: 66 - DNA, 53 bp, Synthetic construct
  source 1..53: mol_type = other DNA
aggacattggcttctgacgcaactgggaccactagcaaccgcaactagccacc

SEQ ID NO: 67 - DNA, 57 bp, Synthetic construct
  source 1..57: mol_type = other DNA
aggaaataagaacataacacgacacatataacacactcaacgacaccagaagccacc

SEQ ID NO: 68 - DNA, 50 bp, Synthetic construct
  source 1..50: mol_type = other DNA
agagagagagagagagagagagagagagagagagagagagagaggccacc

SEQ ID NO: 69 - DNA, 50 bp, Synthetic construct
  source 1..50: mol_type = other DNA
aggatggggcctaacaaaatggcggggccggggccccgggccgggccacc

SEQ ID NO: 70 - DNA, 50 bp, Synthetic construct
  source 1..50: mol_type = other DNA
aggaggactgcgcgaacctgcatagtgatcataaggtcacgatagccacc

SEQ ID NO: 71 - DNA, 50 bp, Synthetic construct
  source 1..50: mol_type = other DNA
aggaggactgggcgaacctgcatagtgatcataaggtcatcatagccacc

SEQ ID NO: 72 - DNA, 58 bp, Synthetic construct
  source 1..58: mol_type = other DNA
aggaaataaggaggactgggcgaacctgcatagtgatcataaggtcatcatagccacc

SEQ ID NO: 73 - DNA, 53 bp, Synthetic construct
  source 1..53: mol_type = other DNA
aggacatttgcttctgacacaactgtgttcactagcaacctcaaacagccacc

SEQ ID NO: 74 - DNA, 53 bp, Synthetic construct
  source 1..53: mol_type = other DNA
agaccataaataaacaacacaaacaacaaacaacccaaataatacccgccacc

SEQ ID NO: 75 - DNA, 57 bp, Synthetic construct
  source 1..57: mol_type = other DNA
aggaaataagacccgggcccgggcccgggcccgggcccgggcccgggccctgccacc

SEQ ID NO: 76 - DNA, 53 bp, Synthetic construct
  source 1..53: mol_type = other DNA
aggtggagagagtgtgtgagatgagtcgcggaatgagccgaccttctgccacc

SEQ ID NO: 77 - DNA, 53 bp, Synthetic construct
  source 1..53: mol_type = other DNA
aggtgagagagtgtagagtgactgaaattgccaaacgtaccgtaatggccacc

SEQ ID NO: 78 - DNA, 53 bp, Synthetic construct
  source 1..53: mol_type = other DNA
aggtgagagagtttaacttgatgaccatgtctaggcataatatgcctgccacc

SEQ ID NO: 79 - DNA, 53 bp, Synthetic construct
  source 1..53: mol_type = other DNA
aggtgggaagagtgtttcagagagagatgttcactaacttgtcaagggccacc

SEQ ID NO: 80 - DNA, 53 bp, Synthetic construct
  source 1..53: mol_type = other DNA
agggggggtgccaggaacttgctactaccagcaccatgctgtaccgcgccacc

SEQ ID NO: 81 - DNA, 53 bp, Synthetic construct
  source 1..53: mol_type = other DNA
aggagcgggcattgcccaggaacttgctactaccagcaccatcctgcgccacc

SEQ ID NO: 82 - DNA, 87 bp, Synthetic construct
  source 1..87: mol_type = other DNA
aggaccacacaaaccgcgtccgccacgctagcggggatcctgtcctttccagatccggaatccgtacacattcaacgcaaggccacc

SEQ ID NO: 83 - DNA, 87 bp, Synthetic construct
  source 1..87: mol_type = other DNA
aggacagacgagaccgcgtccgccccgcgagcacagaccctttcctattccgatctgcatcccttccatagtaaaaggggggccacc

SEQ ID NO: 84 - DNA, 87 bp, Synthetic construct
  source 1..87: mol_type = other DNA
aggacccacgagtgcgcgtgcaccccgcgagcacagagtctcgctgttaactatccgcagcacgtcgacatcactcacctagccacc

SEQ ID NO: 85 - DNA, 87 bp, Synthetic construct
  source 1..87: mol_type = other DNA
aggactcccgagaacgtgtccgcccctcgagcacagagcctcgaccttgccgatccgcctgtcgtccacaccagccgcctagccacc

SEQ ID NO: 86 - DNA, 61 bp, Synthetic construct
  source 1..61: mol_type = other DNA
aggaaataagagaaagaggataagacgactaaggagacatacagaataagaggcagccacc

SEQ ID NO: 87 - DNA, 61 bp, Synthetic construct
  source 1..61: mol_type = other DNA
aggaaataagagaggataagacgactaaggagacatacagaataagaaacaggcagccacc

SEQ ID NO: 88 - DNA, 50 bp, Synthetic construct
  source 1..50: mol_type = other DNA
aggaggactggtcgaacctgcatagtgatcataaggtcagcatagccacc

SEQ ID NO: 89 - DNA, 50 bp, Synthetic construct
  source 1..50: mol_type = other DNA
aggaggactggccgaacctgcatagtgatcataaggtcagcatagccacc

SEQ ID NO: 90 - DNA, 57 bp, Synthetic construct
  source 1..57: mol_type = other DNA
aggaaataagagagaaaagaagagtaagaagaaatataagaccccggcgccgccacc

SEQ ID NO: 91 - DNA, 86 bp, Synthetic construct
  source 1..86: mol_type = other DNA
aggaccgccgagaccgcgtccgccccgcgagcacagagcctcgcctttgccgatccgccgcccgtccacacccgccgccagccacc

SEQ ID NO: 92 - DNA, 53 bp, Synthetic construct
  source 1..53: mol_type = other DNA
aggtgtgagagtttaacttgaagaccatctctagggataatatccctgccacc

SEQ ID NO: 93 - DNA, 53 bp, Synthetic construct
  source 1..53: mol_type = other DNA
agattggagaactttgtttgattatcagaatccttagtagaattggggccacc

SEQ ID NO: 94 - DNA, 53 bp, Synthetic construct
  source 1..53: mol_type = other DNA
aggtgtcagagtttaacttgaagactatttctagggataatatccctgccacc

SEQ ID NO: 95 - DNA, 53 bp, Synthetic construct
  source 1..53: mol_type = other DNA
agagggccggacacagtcccacgctcgcccctccccgtcccgcgggggccacc

SEQ ID NO: 96 - DNA, 53 bp, Synthetic construct
  source 1..53: mol_type = other DNA
aggtgtgagagtttaacttgaagaccatcactcgcgataatatcgcggccacc

SEQ ID NO: 97 - DNA, 53 bp, Synthetic construct
  source 1..53: mol_type = other DNA
aggtcagagagctaagcttaattaccatctctacgactacaagtcgtgccacc

SEQ ID NO: 98 - DNA, 53 bp, Synthetic construct
  source 1..53: mol_type = other DNA
aggtgtgtgagtttaacttgaagaccgtatctagggataatatccctgccacc

SEQ ID NO: 99 - DNA, 53 bp, Synthetic construct
  source 1..53: mol_type = other DNA
agtacaaattccgaaggaacgaatacagaattcaagttattaacttggccacc

SEQ ID NO: 100 - DNA, 53 bp, Synthetic construct
  source 1..53: mol_type = other DNA
aggtgtaagaggttaacttgaagaccatatctagggataatatcgctgccacc

SEQ ID NO: 101 - DNA, 53 bp, Synthetic construct
  source 1..53: mol_type = other DNA
agttttaaaagtttggctacaagagcattagcagcgaagagatcgctgccacc

SEQ ID NO: 102 - DNA, 53 bp, Synthetic construct
  source 1..53: mol_type = other DNA
agctgagagagtttaacttgacggccataactaggattatagctcctgccacc

SEQ ID NO: 103 - DNA, 53 bp, Synthetic construct
  source 1..53: mol_type = other DNA
aggtcttcgaacttatctcaataaccattggccgtattagagtagtagccacc

SEQ ID NO: 104 - DNA, 53 bp, Synthetic construct
  source 1..53: mol_type = other DNA
aggtgagagaggtgagcgcgaaggccaggtcttggcataaggtgcctgccacc

SEQ ID NO: 105 - DNA, 53 bp, Synthetic construct
  source 1..53: mol_type = other DNA
agttcagtataacgcacttggctccaggaacttttagatatagtagagccacc

SEQ ID NO: 106 - DNA, 53 bp, Synthetic construct
  source 1..53: mol_type = other DNA
agcttggagaacttttactagagagtagaaacgttaatagaattggggccacc

SEQ ID NO: 107 - DNA, 53 bp, Synthetic construct
  source 1..53: mol_type = other DNA
aggtcctagagctggactggattaccagtactagtcaatcaatttctgccacc

SEQ ID NO: 108 - DNA, 53 bp, Synthetic construct
  source 1..53: mol_type = other DNA
agttctgtgagtataaatttaggctcattactaggcaccataatccggccacc

SEQ ID NO: 109 - DNA, 53 bp, Synthetic construct
  source 1..53: mol_type = other DNA
aggtgcgggagtggggcgtgctgaccaggtctaggcttagtattcctgccacc

SEQ ID NO: 110 - DNA, 53 bp, Synthetic construct
  source 1..53: mol_type = other DNA
aggtgcgagagtttaacttggagaccatctctagggagaatctccctgccacc

SEQ ID NO: 111 - DNA, 53 bp, Synthetic construct
  source 1..53: mol_type = other DNA
agaataaactagtattcttctggtccccacagactcagagagaacccgccacc

SEQ ID NO: 112 - skipped

SEQ ID NO: 113 - DNA, 61 bp, Synthetic construct
  source 1..61: mol_type = other DNA
agtcctccccatcctctccctctgtccctctgtccctctgaccctgcactgtcccgccacc

SEQ ID NO: 114 - DNA, 39 bp, Synthetic construct
  source 1..39: mol_type = other DNA
agactcttctggtccccacagactcagagagaagccacc

SEQ ID NO: 115 - DNA, 55 bp, Synthetic construct
  source 1..55: mol_type = other DNA
agacgcccctctttctccccaggccgaagcctcgggacggccctggaaggccacc

SEQ ID NO: 116 - DNA, 56 bp, Synthetic construct
  source 1..56: mol_type = other DNA
agaataaactcactatttgttttcgcgcccagttgcaaaaagtgtcgaacgccacc

SEQ ID NO: 117 - DNA, 50 bp, Synthetic construct
  source 1..50: mol_type = other DNA
agctcgtcagctagctagacttctttgctaggacaaccatgttagccacc

SEQ ID NO: 118 - DNA, 50 bp, Synthetic construct
  source 1..50: mol_type = other DNA
agaactataacaatcaaagtccctgtgtccgcgaagccatgagagccacc

SEQ ID NO: 119 - DNA, 50 bp, Synthetic construct
  source 1..50: mol_type = other DNA
aggctagcagcatagttagtctacaatattacactaacaagagagccacc

SEQ ID NO: 120 - DNA, 50 bp, Synthetic construct
  source 1..50: mol_type = other DNA
agtagcataagtggtccaggactcttagttcgcaaataatgcgcgccacc

SEQ ID NO: 121 - DNA, 50 bp, Synthetic construct
  source 1..50: mol_type = other DNA
agacacttagcttacgtcatttcctggcaattagtaactgaatagccacc

SEQ ID NO: 122 - DNA, 50 bp, Synthetic construct
  source 1..50: mol_type = other DNA
agcaattcacactttccattgcggaagaattctttagcatccaagccacc

SEQ ID NO: 123 - DNA, 50 bp, Synthetic construct
  source 1..50: mol_type = other DNA
agtatccagattagcaaaacccaaacaatcttgtaagaatccaagccacc

SEQ ID NO: 124 - DNA, 50 bp, Synthetic construct
  source 1..50: mol_type = other DNA
agtggaccacgcagaagattgttgagactattatgctagtccgtgccacc

SEQ ID NO: 125 - DNA, 50 bp, Synthetic construct
  source 1..50: mol_type = other DNA
agcctactcgtctttagagagagatacacaatacttatatgagagccacc

SEQ ID NO: 126 - DNA, 50 bp, Synthetic construct
  source 1..50: mol_type = other DNA
agtgacaaaagtagccaaattctcctagcctcaaccaagaatcggccacc

SEQ ID NO: 127 - DNA, 50 bp, Synthetic construct
  source 1..50: mol_type = other DNA
agtgtctatcccattcattccgtgatctactacttagcaaccgtgccacc

SEQ ID NO: 128 - DNA, 50 bp, Synthetic construct
  source 1..50: mol_type = other DNA
agtgtccatctttggcaagttgctctagcatgataattttgagggccacc

SEQ ID NO: 129 - DNA, 50 bp, Synthetic construct
  source 1..50: mol_type = other DNA
agtctgaaactaatccagttggtgaggacattacaagagtctcggccacc

SEQ ID NO: 130 - DNA, 50 bp, Synthetic construct
  source 1..50: mol_type = other DNA
agacgtctaactgttccctggactaatatacaattgaaatgaaagccacc

SEQ ID NO: 131 - DNA, 50 bp, Synthetic construct
  source 1..50: mol_type = other DNA
aggtggttcttctgcaaattagcgaccaaataatgagaatcagcgccacc

SEQ ID NO: 132 - DNA, 50 bp, Synthetic construct
  source 1..50: mol_type = other DNA
agatcaaatactaacgaattcgtgagcgcttaatcgcgacgtttgccacc

SEQ ID NO: 133 - DNA, 50 bp, Synthetic construct
  source 1..50: mol_type = other DNA
agaaacaccagtaggcaacattttgtagcaactctgttaagagagccacc

SEQ ID NO: 134 - DNA, 50 bp, Synthetic construct
  source 1..50: mol_type = other DNA
agtatatagaatatctagttgatagaagaccaatgagtacagctgccacc

SEQ ID NO: 135 - DNA, 50 bp, Synthetic construct
  source 1..50: mol_type = other DNA
agacaaattgctaccttagaaaccgagagaagcgtatcatcagagccacc

SEQ ID NO: 136 - DNA, 50 bp, Synthetic construct
  source 1..50: mol_type = other DNA
agtcgctaactgaaccacttaccgataattcatatataaacgctgccacc

SEQ ID NO: 137 - DNA, 50 bp, Synthetic construct
  source 1..50: mol_type = other DNA
agtccatagcgctaattagctatttacattatcctcacagcaacgccacc

SEQ ID NO: 138 - DNA, 50 bp, Synthetic construct
  source 1..50: mol_type = other DNA
aggccgtcacctaaagaaatacttgtgatcatttatcaataattgccacc

SEQ ID NO: 139 - DNA, 50 bp, Synthetic construct
  source 1..50: mol_type = other DNA
agtaaataaccgtgttcggtagctcctgtggtaaggactacatagccacc

SEQ ID NO: 140 - DNA, 50 bp, Synthetic construct
  source 1..50: mol_type = other DNA
agtatcagagtgaaccctgagatactaatatcctgagagtttaagccacc

SEQ ID NO: 141 - DNA, 50 bp, Synthetic construct
  source 1..50: mol_type = other DNA
aggaacaaccccgagaacaaggttttcttatcatccggtttcgagccacc

SEQ ID NO: 142 - DNA, 50 bp, Synthetic construct
  source 1..50: mol_type = other DNA
agctaatctgcttatctgtttctcgatataattaaagcattacagccacc

SEQ ID NO: 143 - DNA, 50 bp, Synthetic construct
  source 1..50: mol_type = other DNA
agtaactaactgattggcttagctctgtaagcaacccaattctggccacc

SEQ ID NO: 144 - DNA, 50 bp, Synthetic construct
  source 1..50: mol_type = other DNA
agtctcttcactgctttcattctttataaagttccaaaatgggtgccacc

SEQ ID NO: 145 - DNA, 50 bp, Synthetic construct
  source 1..50: mol_type = other DNA
agaatatatagcctctaattgttggccattttaacctctgccgcgccacc

SEQ ID NO: 146 - DNA, 50 bp, Synthetic construct
  source 1..50: mol_type = other DNA
aggcggtccaacatcttgtgactttagcttgcaaagttatgacagccacc

SEQ ID NO: 147 - DNA, 50 bp, Synthetic construct
  source 1..50: mol_type = other DNA
agtttcaaactttctctatagagaataatattagccttctctgcgccacc

SEQ ID NO: 148 - DNA, 50 bp, Synthetic construct
  source 1..50: mol_type = other DNA
agtaccggtattacgcagttcgttttattatttcgacaatgtgcgccacc

SEQ ID NO: 149 - DNA, 50 bp, Synthetic construct
  source 1..50: mol_type = other DNA
aggactattttagcattaggtgatttggtaacaccgctttagacgccacc

SEQ ID NO: 150 - DNA, 50 bp, Synthetic construct
  source 1..50: mol_type = other DNA
aggagctgacccattagaatactgctcgtttaaactgattccctgccacc

SEQ ID NO: 151 - DNA, 50 bp, Synthetic construct
  source 1..50: mol_type = other DNA
aggctttgagacaataatttagttataagagaacccaaaaccaagccacc

SEQ ID NO: 152 - DNA, 50 bp, Synthetic construct
  source 1..50: mol_type = other DNA
aggaagatacctagctgagttgtctctatagaagagaaatgagagccacc

SEQ ID NO: 153 - DNA, 50 bp, Synthetic construct
  source 1..50: mol_type = other DNA
agcgagtaagcttgttctgtcatctaatatccatgcaaatggcagccacc

SEQ ID NO: 154 - DNA, 50 bp, Synthetic construct
  source 1..50: mol_type = other DNA
agtaaattagccaatcaaggtaactgtaaccttgtaccaagggagccacc

SEQ ID NO: 155 - DNA, 50 bp, Synthetic construct
  source 1..50: mol_type = other DNA
aggatacgagtgatacacattgtgaactacctatccagagttgcgccacc

SEQ ID NO: 156 - DNA, 50 bp, Synthetic construct
  source 1..50: mol_type = other DNA
agtgccactgtctgtgaaagtgatagactggccagctaatgagagccacc

SEQ ID NO: 157 - DNA, 50 bp, Synthetic construct
  source 1..50: mol_type = other DNA
aggatttcattgaacacattgcactgtttcaagttccgagcagcgccacc

SEQ ID NO: 158 - DNA, 50 bp, Synthetic construct
  source 1..50: mol_type = other DNA
agcacaatacgaacccagattctcttggtaccgaactgacaattgccacc

SEQ ID NO: 159 - DNA, 50 bp, Synthetic construct
  source 1..50: mol_type = other DNA
agtttggttcacttactcctggggagcatataatttatagaccagccacc

SEQ ID NO: 160 - DNA, 50 bp, Synthetic construct
  source 1..50: mol_type = other DNA
agctttagtcgcacccaacatattcgagtaggtaaaccatgccagccacc

SEQ ID NO: 161 - DNA, 50 bp, Synthetic construct
  source 1..50: mol_type = other DNA
agtatcatacttagacattgagctgaacgattaactgcagtccagccacc

SEQ ID NO: 162 - DNA, 50 bp, Synthetic construct
  source 1..50: mol_type = other DNA
agtactacacataaccgagtctgtagagtcataatatatctagagccacc

SEQ ID NO: 163 - DNA, 50 bp, Synthetic construct
  source 1..50: mol_type = other DNA
agatacggccacaactgaatcttttcgatagacctcagctagttgccacc

SEQ ID NO: 164 - DNA, 50 bp, Synthetic construct
  source 1..50: mol_type = other DNA
aggagatacaatacaggagatcgaaggacagtgcatctagcagtgccacc

SEQ ID NO: 165 - DNA, 50 bp, Synthetic construct
  source 1..50: mol_type = other DNA
agtaacacatgttgaacatttgttcgggtattagagtaagtgcggccacc

SEQ ID NO: 166 - DNA, 50 bp, Synthetic construct
  source 1..50: mol_type = other DNA
agttccacataaaggtagttgaacgtagtaatttaagatcccgagccacc

SEQ ID NO: 167 - DNA, 50 bp, Synthetic construct
  source 1..50: mol_type = other DNA
agtgtaaacattacccaactggttataattctctagcgcacggagccacc

SEQ ID NO: 168 - DNA, 50 bp, Synthetic construct
  source 1..50: mol_type = other DNA
agggtctaactaacaccaaggaagattaagtggtcagaatgcttgccacc

SEQ ID NO: 169 - DNA, 50 bp, Synthetic construct
  source 1..50: mol_type = other DNA
agagccgctcgtttcatcgctattgtagaaccacaatatttgcagccacc

SEQ ID NO: 170 - DNA, 92 bp, Synthetic construct
  source 1..92: mol_type = other DNA
agaacccgaacgcccttccgtcgctccaagggctagtggagcgttgctacccgccgacacttgctactaccagcaccaccgtcttcgccacc

SEQ ID NO: 171 - DNA, 92 bp, Synthetic construct
  source 1..92: mol_type = other DNA
agatcagacggtacatcgctacaagcgctagtacggagtgtaagccacaatttgaaacttgctactaccaggctgcgtagggattcgccacc

SEQ ID NO: 172 - DNA, 92 bp, Synthetic construct
  source 1..92: mol_type = other DNA
agtaggaacgccgctccgaggcctcattgtagcaggcgataccgtggcaccctacctcccaaccatccttcgccgccgcagtcttcgccacc

SEQ ID NO: 173 - DNA, 92 bp, Synthetic construct
  source 1..92: mol_type = other DNA
agcgtcgcgtcgttcagacgcaattacgtaatggcgtgggagaacgaacgtgccacccgcatatgcttctaccagtcggagtcttcgccacc

SEQ ID NO: 174 - DNA, 92 bp, Synthetic construct
  source 1..92: mol_type = other DNA
agtcggatggcctgatacaaagcgaccgtcgttccgacgcccaagttaccacgaatcaatgatgcggagaccgcgttacggacttcgccacc

SEQ ID NO: 175 - DNA, 92 bp, Synthetic construct
  source 1..92: mol_type = other DNA
agtatgacactccgaaggcctcaagtccaataaccgcacgtagccgacaaccttctaccacgcgtctaccaagtgtcccggacttcgccacc

SEQ ID NO: 176 - DNA, 92 bp, Synthetic construct
  source 1..92: mol_type = other DNA
agaaacgcgaataggagaatccgacgctacgcccttagctgcacattcggtctacctccccccactgagtctacgcttctaacttcgccacc

SEQ ID NO: 177 - DNA, 92 bp, Synthetic construct
  source 1..92: mol_type = other DNA
agggatagacgtccccgtcgctcgaagcgcgcgttgttgtggctgtcaggggtctaggatccagtttcggtgccgctgcagtcttcgccacc

SEQ ID NO: 178 - DNA, 92 bp, Synthetic construct
  source 1..92: mol_type = other DNA
aggcgttgaggacggcgctgcgtcgtgcataaaaccccacctagcttcgcgtgtgctctactgaactactacccgccgcagtcttcgccacc

SEQ ID NO: 179 - DNA, 92 bp, Synthetic construct
  source 1..92: mol_type = other DNA
agtgtaaacgtcactacgtcaacgctctcgacggtcgtctagcacagttgaccgtactaccccgcgtctaataattcccaatcttcgccacc

SEQ ID NO: 180 - DNA, 92 bp, Synthetic construct
  source 1..92: mol_type = other DNA
agatctttaaacgccggtgtacgcacggcttagggagtgccgtccaaattactgctctgtgtgccatcctgccgagtaaagtcttcgccacc

SEQ ID NO: 181 - DNA, 92 bp, Synthetic construct
  source 1..92: mol_type = other DNA
agtctgctgcggtattgtctctttgttaaaaggctagaactccagtcccgagtccgagtccttcctattctttgtgaccggtcttcgccacc

SEQ ID NO: 182 - DNA, 92 bp, Synthetic construct
  source 1..92: mol_type = other DNA
agcagcaccgacacccaccgttcagacgctcctgaatctgtattcaccccattggtcaatgacccctgaccgtgtgtaccagcttcgccacc

SEQ ID NO: 183 - DNA, 92 bp, Synthetic construct
  source 1..92: mol_type = other DNA
agcagggtgtccttcagacgctcctgaaaggaaggtggtctctcgcagttgtacctccctacctattgttctactactcatcctttgccacc

SEQ ID NO: 184 - DNA, 92 bp, Synthetic construct
  source 1..92: mol_type = other DNA
agactgagtctcgcatcgtcgcttctaaaaccagcagtggaggaatactacgatactacaattaccacgttccacccgcagtcttcgccacc

SEQ ID NO: 185 - DNA, 92 bp, Synthetic construct
  source 1..92: mol_type = other DNA
agcgcaaaaaaaatcgccaccgacggccagacgcaactataacgtggaccaggttcccccccgcgtctacgtacgccgcggtcttcgccacc

SEQ ID NO: 186 - DNA, 92 bp, Synthetic construct
  source 1..92: mol_type = other DNA
agcctgaattctaacgctcagtcgcacctggatcaaaatgcatccaggactaaactagttgtacattcgtaccagccgcagtcttcgccacc

SEQ ID NO: 187 - DNA, 92 bp, Synthetic construct
  source 1..92: mol_type = other DNA
agggttgggcaaagcaagatggccatcgcagcaacggcgatcgttactctactacgggtctaccaagcggcccaggcgcagtcttcgccacc

SEQ ID NO: 188 - DNA, 92 bp, Synthetic construct
  source 1..92: mol_type = other DNA
aggacattaagtgcaagtgtcgtacaacgctgaagaccctcaaagaacacgacactgcgcaactgtacgtactactaccagtcttcgccacc

SEQ ID NO: 189 - DNA, 92 bp, Synthetic construct
  source 1..92: mol_type = other DNA
agccgagtaagtgcaagcgccgcttcttcagcccggatggctatatcattcgccatacttgcgtttcgacgccgggcccagtcttcgccacc

SEQ ID NO: 190 - DNA, 92 bp, Synthetic construct
  source 1..92: mol_type = other DNA
agatcgcactgcgtgtaagcagcctctagccgcacgcgtcgttgcaatttttcgaccgtaccattgatattcgcgccgcagtcttcgccacc

SEQ ID NO: 191 - DNA, 92 bp, Synthetic construct
  source 1..92: mol_type = other DNA
agagggtaaccgtaacggtctatcgctgcatcgacaataggaccgctggttagtctacgtaccggaccactacctcctcagtcttcgccacc

SEQ ID NO: 192 - DNA, 92 bp, Synthetic construct
  source 1..92: mol_type = other DNA
agtgcatccgtcgctccaagtgcctgtgaccctcagaggtttgcaccataataacaggagtacctctaccagccgcccaagtcttcgccacc

SEQ ID NO: 193 - DNA, 92 bp, Synthetic construct
  source 1..92: mol_type = other DNA
aggacactttaccgtcacaaggtgtctttaactgattaactgcaagggagtcctctcctacgagtcaatgatcttccgtggacttcgccacc

SEQ ID NO: 194 - DNA, 92 bp, Synthetic construct
  source 1..92: mol_type = other DNA
agcgccgttgtagagaatactacgctcaaacgctagaaccgcaactaccagatcaccccctacctatgcttctacctcccgtcctcgccacc

SEQ ID NO: 195 - DNA, 92 bp, Synthetic construct
  source 1..92: mol_type = other DNA
aggtaggtgtagacacactaaattctttgggtggagtcttcccacacccgaagcgcaagaagcatagtgcatctcacactcacctagccacc

SEQ ID NO: 196 - DNA, 92 bp, Synthetic construct
  source 1..92: mol_type = other DNA
agactaaaaggacgccctctaaagggagaactctgcataaggcaggcgggcgagaccaaatcgcaggccttggctccttgaaccaagccacc

SEQ ID NO: 197 - DNA, 92 bp, Synthetic construct
  source 1..92: mol_type = other DNA
agtaaagacccagcagaaatctttgcatagaatgatgactccctcgatagaattttagcccactgcatccagccaataggagcccagccacc

SEQ ID NO: 198 - DNA, 92 bp, Synthetic construct
  source 1..92: mol_type = other DNA
aggactctgcttgagtgtgctctttgtatcagtgttggctggtctctgagaggaaggccttggctccttgaacttttgtctctcttgccacc

SEQ ID NO: 199 - DNA, 92 bp, Synthetic construct
  source 1..92: mol_type = other DNA
agaaggaccattaaaagtatgcccagtggagtctcctgaaataagtgctccttgaacttttggcaggccttggctccttgtacttggccacc

SEQ ID NO: 200 - DNA, 92 bp, Synthetic construct
  source 1..92: mol_type = other DNA
agaacgtgcacaatttgattcactccctcaagacggagcactctgcaggccttggctccttgaactgttggcccgcatgtcagcaggccacc

SEQ ID NO: 201 - DNA, 92 bp, Synthetic construct
  source 1..92: mol_type = other DNA
agatctagtgtataacttggataggaaaaaaattctttgggtggtgctattgctgacaccaactgaatccagccaataggagcccagccacc

SEQ ID NO: 202 - DNA, 92 bp, Synthetic construct
  source 1..92: mol_type = other DNA
agtcccactgcagtaaagggtaaacttcatgagatggggctgtttctggccttggctccttgaacttttaaattagccatctcatggccacc

SEQ ID NO: 203 - DNA, 92 bp, Synthetic construct
  source 1..92: mol_type = other DNA
agtgtactcctctttgcctggactaagcactgcctctcaaaggagtaagccttggctccttgaacttttttgctccatcgcagtaggccacc

SEQ ID NO: 204 - DNA, 92 bp, Synthetic construct
  source 1..92: mol_type = other DNA
aggaaaaaaaaatctctctcgggtggagtcttcttcaatgcttgcctaaaaagactaggctcttggcgcatcgcgccacacacctagccacc

SEQ ID NO: 205 - DNA, 92 bp, Synthetic construct
  source 1..92: mol_type = other DNA
agcaaaaaaaaaaaaacccctcgggtggagtcctcgcaaagattacgttattccagatccttaagctgacagccaataggagcccagccacc

SEQ ID NO: 206 - DNA, 92 bp, Synthetic construct
  source 1..92: mol_type = other DNA
aggatatcttatttaagtactcctcgggtggagtcctcacaggcctaggctccttgaggcaattgccttccgtcaataggagcccagccacc

SEQ ID NO: 207 - DNA, 92 bp, Synthetic construct
  source 1..92: mol_type = other DNA
agaccctgagtagaagagttaaataaaacactagtccactcgaggtctaaccagcaggccttggctccttgaacttatatcacctagccacc

SEQ ID NO: 208 - DNA, 92 bp, Synthetic construct
  source 1..92: mol_type = other DNA
agatactactccctgaatactctgtggggagtcctctcatacaggcaggcctaggctccttgaactttaggccgccatcgaatgaggccacc

SEQ ID NO: 209 - DNA, 92 bp, Synthetic construct
  source 1..92: mol_type = other DNA
agacacctcgggtggagtcctgagaaggtatgaattataaaacgtagcagtacatcaaacatacggccttggctccttgaacttttgccacc

SEQ ID NO: 210 - DNA, 92 bp, Synthetic construct
  source 1..92: mol_type = other DNA
aggaccccgaatacctctcgggtggagtctcctcccagacaaggcttaccgtaagaatattagattcctgaggcaataggagcccagccacc

SEQ ID NO: 211 - DNA, 92 bp, Synthetic construct
  source 1..92: mol_type = other DNA
agcctgccccaaactaactcctcgggtggagtctcctgaatgaggacgcgctgtttaatgaaacaaaacgaaccaataggagcccagccacc

SEQ ID NO: 212 - DNA, 92 bp, Synthetic construct
  source 1..92: mol_type = other DNA
aggtctctcaacatacctctcgggtggagtcttctgaatggatagagttttgctggagatgaagccattggctgaaagcacacctagccacc

SEQ ID NO: 213 - DNA, 92 bp, Synthetic construct
  source 1..92: mol_type = other DNA
agctctcgggtggagtcttctaaaaatttaacaaggctaatcctatggtggttgatgtcgaaatcacatagcgcaataggagcccagccacc

SEQ ID NO: 214 - DNA, 92 bp, Synthetic construct
  source 1..92: mol_type = other DNA
agtctatactcgaaggaaaagctctcgggtggagtcttctgcctataatagcacaatcaaaaggcatcataagtaataggagcccagccacc

SEQ ID NO: 215 - DNA, 92 bp, Synthetic construct
  source 1..92: mol_type = other DNA
agatgaggaagcttgtccgaaggcatagtccagtcctcccttctttaggccttggctccttgaacttttggaagtaattagagtaggccacc

SEQ ID NO: 216 - DNA, 92 bp, Synthetic construct
  source 1..92: mol_type = other DNA
agaacggaaggactctgtggagaaacaaaactcttcgggtggagtctacagttggatgaagaggaatagaaggcaataggagcccagccacc

SEQ ID NO: 217 - DNA, 92 bp, Synthetic construct
  source 1..92: mol_type = other DNA
agtggcgctaaactaactcctcgggtggagtctcgcctctctatacatggtaagcattagaaccatatctctttcacactcacctagccacc

SEQ ID NO: 218 - DNA, 92 bp, Synthetic construct
  source 1..92: mol_type = other DNA
agatctcgggtggagtcctgaggatgaaggtgatgaacatcctacctagaggcgtaatatcactgcatccagccaataggagcccagccacc

SEQ ID NO: 219 - DNA, 92 bp, Synthetic construct
  source 1..92: mol_type = other DNA
agtcctccgggtggagtcctctcaaagaagaagaaactaaagaaaaaggccttggctccttgaacttttggccgccatgtagagaggccacc

SEQ ID NO: 220 - DNA, 92 bp, Synthetic construct
  source 1..92: mol_type = other DNA
aggagcatggtgacgataatcttgagggtgagctgagattcagaggataacagaaggccttggctccttgaacttttgcccgttatgccacc

SEQ ID NO: 221 - DNA, 92 bp, Synthetic construct
  source 1..92: mol_type = other DNA
agaactgctaaactgccccagaaaattgtcatgaaaaatactaatgagaaggccttggctccttgaacttttggccgccatgtgtcgccacc

SEQ ID NO: 222 - DNA, 92 bp, Synthetic construct
  source 1..92: mol_type = other DNA
agatctctctcgggtggaatcctgggaaggtatttattaacttaacgatggccttggctccttgaacttttggccgtccttcagaggccacc

SEQ ID NO: 223 - DNA, 92 bp, Synthetic construct
  source 1..92: mol_type = other DNA
agaataattcttcgcgtgcagtattcagacggagtagtcttcccacacctcaggccttggctccttgaacttttggccgccatgtcgccacc

SEQ ID NO: 224 - DNA, 92 bp, Synthetic construct
  source 1..92: mol_type = other DNA
agtcaaagacctcgggtggagtcttcaggcgaagatctctctgtttgcgtcaggccttggctccttgaacttttggccaccatgtggccacc

SEQ ID NO: 225 - DNA, 92 bp, Synthetic construct
  source 1..92: mol_type = other DNA
agctctggggtggattcttctagatgaaacacactaaattaaactattaacgaaagccttggctccttgaacttttggccgccatggccacc

SEQ ID NO: 226 - DNA, 92 bp, Synthetic construct
  source 1..92: mol_type = other DNA
agaaactttctcgggtggagtctttagatatccatcctagccagacgactgaaggccttggctccttgaacttttggcaacttgtggccacc

SEQ ID NO: 227 - DNA, 92 bp, Synthetic construct
  source 1..92: mol_type = other DNA
agccctggggtggagtcctctgcactcacatcatttcaacctaaagcagatcacgatatctcaggccttggctccttgaacttttggccacc

SEQ ID NO: 228 - DNA, 92 bp, Synthetic construct
  source 1..92: mol_type = other DNA
agctctagggtagcgtcctctgaacaaactacagacctggcttagatcatagaaggccttggctccttgaacttttggacgccatggccacc

SEQ ID NO: 229 - DNA, 92 bp, Synthetic construct
  source 1..92: mol_type = other DNA
aggtaaacaaaaaaaaaagaactctctcgggtggagtcttcggacgtacggaaggccttggctccttgaacttttggccgccattggccacc

SEQ ID NO: 230 - DNA, 92 bp, Synthetic construct
  source 1..92: mol_type = other DNA
agacactaagttctcgcccggagaagtctcatcaaataattctcgctgcaggccttggctccttgaacttttggccgccaagtgtcgccacc

SEQ ID NO: 231 - DNA, 92 bp, Synthetic construct
  source 1..92: mol_type = other DNA
agccctgtctgaaaggcctcgggtggagtcctctcacgcccacgctagtcgacatctcaggccttggctccttgaacttttggccggccacc

SEQ ID NO: 232 - DNA, 92 bp, Synthetic construct
  source 1..92: mol_type = other DNA
agtcctctgagacaggactcgggtggaggcttacttgttaattaggcaccgtcatctcaggccttggctccttgaacttttggactgccacc

SEQ ID NO: 233 - DNA, 92 bp, Synthetic construct
  source 1..92: mol_type = other DNA
agattgtctctcgggtgcaatctacggactaaaaagacctctcagttacggaaggccttggctccttgaacttttggccgccattggccacc

SEQ ID NO: 234 - DNA, 92 bp, Synthetic construct
  source 1..92: mol_type = other DNA
agtgtatggatctctcagaagtaacccttcgggtggagtcttcccacttgaacttttggccgccaccgcaggccttggctccttgagccacc

SEQ ID NO: 235 - DNA, 92 bp, Synthetic construct
  source 1..92: mol_type = other DNA
agaactctcgtgaggtcctctgaactcacttgaattcttgtcctgcgcattcgcgataccttaggccttggctccttgaacttttggccacc

SEQ ID NO: 236 - DNA, 92 bp, Synthetic construct
  source 1..92: mol_type = other DNA
aggactaaagtactccccaggcgaattcacgtcaaaagacctctcaggaaggccttggctccttgaacttttggccgccatgtgtcgccacc

SEQ ID NO: 237 - DNA, 92 bp, Synthetic construct
  source 1..92: mol_type = other DNA
agccaaaagaacttaaatctctctcgggtggagtcttatgaaaggattacagaatacatctcaggccttggctcctcgaccttttggccacc

SEQ ID NO: 238 - DNA, 92 bp, Synthetic construct
  source 1..92: mol_type = other DNA
agtaataaatctctctcgggtggagtctcatgcacacgtgctaggcgagtttttgatcccgaggctctgtgagtaccacacacctagccacc

SEQ ID NO: 239 - DNA, 92 bp, Synthetic construct
  source 1..92: mol_type = other DNA
agtcctcctgaatgaccggagaagtcctctcaaaaattgacataggggaaggccttggctccttgaacttttggccgcaacgtgaggccacc

SEQ ID NO: 240 - DNA, 92 bp, Synthetic construct
  source 1..92: mol_type = other DNA
agcacagaaaaagtacttctctgaagtctcataacaaaaccagacaggaaggccttggctccttgaacttttggccaacatttcccgccacc

SEQ ID NO: 241 - DNA, 92 bp, Synthetic construct
  source 1..92: mol_type = other DNA
agactacctgtgaactcggagaaggagttcccccaggcttgcatacagtaggccttggctccttgaacttttggccgccatgacgtgccacc

SEQ ID NO: 242 - DNA, 92 bp, Synthetic construct
  source 1..92: mol_type = other DNA
agaccgacagaggactcgcagaaggacttcctattgaatctattgtggaaggccttggctccttgaacttttgaccgccatgtgaggccacc

SEQ ID NO: 243 - DNA, 92 bp, Synthetic construct
  source 1..92: mol_type = other DNA
agaatctctctcgggtggagtcttcccacagttcacacataaaatttatcgcgaggctctgtgagtaccacacagtgggattactagccacc

SEQ ID NO: 244 - DNA, 92 bp, Synthetic construct
  source 1..92: mol_type = other DNA
aggctctcgtgtggagtcttgtcaaataaaactttgggtggtggacggccttggctccttgaacttttggccgccatgtggagcaggccacc

SEQ ID NO: 245 - DNA, 92 bp, Synthetic construct
  source 1..92: mol_type = other DNA
agatctctcacgggttgaatcatgtgaaagtaaactaaatggatcggcaggccttggctccttgaacttttggccgaccaccataggccacc

SEQ ID NO: 246 - DNA, 92 bp, Synthetic construct
  source 1..92: mol_type = other DNA
agacttaaatctctctcgggtggagtcttctggtaaaagaaaaaattctccaagagcaaacagcaggccttggctccttgaacaaagccacc

SEQ ID NO: 247 - DNA, 76 bp, Synthetic construct
  source 1..76: mol_type = other DNA
agataatgcaaaaatcaaaatcaatcatcatcacaacatcaacaatcaatcatcaacacatcatcaagacgccacc

SEQ ID NO: 248 - DNA, 69 bp, Synthetic construct
  source 1..69: mol_type = other DNA
agataatcactcctccccatcctctccctctgtccctctgtccctctgaccctgcactgtcccgccacc

SEQ ID NO: 249 - DNA, 80 bp, Synthetic construct
  source 1..80: mol_type = other DNA
agataatacagcagttacactgcggcgggcgtctgttctagtgtttgagccgtcgtgcttcaccggtctacctcgccacc

SEQ ID NO: 250 - DNA, 166 bp, Synthetic construct
  source 1..166: mol_type = other DNA
agataatctttctgcagcaggaaccgcggctgctggacaagaggggtgcggtggatactgacctttgctccggcctcgtgtagcgtgaagacacagcgcatctccccgctgtaggcttcctcccacagaacccgtttcgggcctcagagcgtctggtgaggccacc

SEQ ID NO: 251 - DNA, 102 bp, Synthetic construct
  source 1..102: mol_type = other DNA
agataattctacagcattgtcccagatatagccgttggtacaaagcggggatctgacgagcttttctctacttgtgtcactaacggaccgtttatcgccacc

SEQ ID NO: 252 - DNA, 190 bp, Synthetic construct
  source 1..190: mol_type = other DNA
agataatcgccgccccttccgaggctaaatcggctgcgttcctctcggaacgcgccgcagaaggggtcctggtgacgagtcccgcgttctctccttgaatccactcgccagcccgccgccctctgccgccgcaccctgcacacccgcccctctcctgtgccaggaacttgctactaccagcaccgccacc

SEQ ID NO: 253 - DNA, 130 bp, Synthetic construct
  source 1..130: mol_type = other DNA
agataatcacatatgtatccacagcgcctagaacactgcctgtaacgtggaaggtgttcgatctatagagttttgtcgaatgaatgaatgaagccgactagtgcacagggagtgcagcggcgcggccacc

SEQ ID NO: 254 - DNA, 68 bp, Synthetic construct
  source 1..68: mol_type = other DNA
agataatacttccccgtcaccagctcctgtgcctgccagtcggtgcccctcccgctccagccgccacc

SEQ ID NO: 255 - DNA, 85 bp, Synthetic construct
  source 1..85: mol_type = other DNA
agataatctcagtcgccgctgccagctctcgcactctgttcttccgccgctccgccgtcgcgtttctctgccggtcgcagccacc

SEQ ID NO: 256 - DNA, 91 bp, Synthetic construct
  source 1..91: mol_type = other DNA
agataatctcattgaactcgcctgcagctcttgggttttttgtggcttccttcgttattggagccaggcctacaccccagcaaccgccacc

SEQ ID NO: 257 - DNA, 129 bp, Synthetic construct
  source 1..129: mol_type = other DNA
agataatagtatctgtgggtacccggagcacggagatctcgccggctttacgttcacctcggtgtctgcagcaccctccgcttcctctcctaggcgacgagacccagtggctagaagttcaccgccacc

SEQ ID NO: 258 - DNA, 102 bp, Synthetic construct
  source 1..102: mol_type = other DNA
agataatccagttactctagcgcgccaggccgaaccgcagcttcttggcttaggtacttctactcacagcggccgattccgaggccaactccagcagccacc

SEQ ID NO: 259 - DNA, 225 bp, Synthetic construct
  source 1..225: mol_type = other DNA
agataatagtgaattcaccaccccagctctctgcggattgagctagtgcatcttttggaaaaatacctattatttgtgaggtcttcaaaagttgtgacagccctgattcagatcaggtagctttcacatttgtccattgctattttgagaaaaagcatttaaaaacaatatgtggtcttatttttggtatacgggattttaaaaaaaaggtgctgccacgccacc

SEQ ID NO: 260 - DNA, 207 bp, Synthetic construct
  source 1..207: mol_type = other DNA
agataatgcgagtaagtgcaggtgccggtggcgcacggggctcgggtagttctgggaacctctgggcggtcctgggactgaggagtcggcgccttcctcaagaatgcctgggacaaggagccagtgctggtcgtgtccttcgtcgtcgggggcctcgctgtaattctgcccccattgagcccctacttcaagtactccgtcgccacc

SEQ ID NO: 261 - DNA, 266 bp, Synthetic construct
  source 1..266: mol_type = other DNA
agataataaaggttcctgcctctcctcggccaggcggaacctctctgctgggcccggtggccgcaaaagaactttctttctcccgcccgaacggtcgccgcggccaactgcctcgcccgcctggcagcctaaccctccttctcttcttctcctctccggcttcgcgcggccctgcctccctctcgcccggcggcatccgcttgctgctgccaccgcctcctcatcttctgcccggccaaccggcctgccccgctgcagtggccacc

SEQ ID NO: 262 - DNA, 79 bp, Synthetic construct
  source 1..79: mol_type = other DNA
agataatagaacagacagacctactgacacaggggaggctctgtgagtaccacacagtggggagggggtgggggccacc

SEQ ID NO: 263 - DNA, 81 bp, Synthetic construct
  source 1..81: mol_type = other DNA
agataatcataaaaccagctgagtatttgtgccaggaagactgcgtgcagaaggccttggctccttgaacttttggccacc

SEQ ID NO: 264 - DNA, 64 bp, Synthetic construct
  source 1..64: mol_type = other DNA
agataatagcaaggctcagcctcaagattcacagcatctcagacacagcctaggccgcgccacc

SEQ ID NO: 265 - DNA, 216 bp, Synthetic construct
  source 1..216: mol_type = other DNA
agataataatctagttctctgtgttcgggcggggggagtcgaactcgtggggtgtcagtatcccgggttcgtctccccactaccggttcccaccactgagtctgggggcgaaggaaggagccagagtgcaattgcagatccagaccccagagtcagaaggagtgagaaccctgacccctaatcccactgcatccagccaataggagcccagccacc

SEQ ID NO: 266 - DNA, 397 bp, Synthetic construct
  source 1..397: mol_type = other DNA
agataataatcagggctcaactatggctgcctcctcctcttcctctggatcaggaggcgcagggagcagggagatgtagacctcattgacctctgtgtccaaatgtcggccaccccgaggtgcttccaggtttaagatgccatcatgggagagagcagctcggactcgccaggggtcgacatcagcaggcaggacatagctcagtgagtactgggtatgtgtcacattgccaaatcccggatcacaagtctccatgaactgctggtgagctaggataataaaacccctgacatcaccattccagaagcttcacaagactgcatatataaggggctggctgtagctgcagctgaaggagctgaccagccagctgacccctcacactcacctagccacc

SEQ ID NO: 267 - DNA, 204 bp, Synthetic construct
  source 1..204: mol_type = other DNA
agataatgtttccagggggagaactctgcagaggagaggcaaagttcaacaggacaacaaagaagcagcagaaacagccaggaaaacgaagagcccccaggttttgacacagaaccctccaccagagttctcaaatccccaaatactctgcaaagtgcaacttgacgggaacttttcaactgacttttacttttgacggccacc

SEQ ID NO: 268 - DNA, 103 bp, Synthetic construct
  source 1..103: mol_type = other DNA
agataattcctcctcgcctgccaccggtgcacccagtccgctcacccagcccagtccgtccggtcctcaccgcctgccggccggcccaccccccaccgccacc

SEQ ID NO: 269 - DNA, 215 bp, Synthetic construct
  source 1..215: mol_type = other DNA
agataatggtgagctgcatgatgtcttacatttcatgagcttcctctaaatctagttgtatttttcgtctgaggggggctgaggctgaggtggcctccttgaaccgtaggatccagctggttgaagaagagctggaccgtgctcaggagcgcctggccactgccctgcaaaagctggaagaagctgaaaaagctgctgatgagagtgaggccacc

SEQ ID NO: 270 - DNA, 82 bp, Synthetic construct
  source 1..82: mol_type = other DNA
agataatgcctgcagccggtgctgtccacagggagctccagcccttctcacactcgacccgcagaaaccacccaccgccacc

SEQ ID NO: 271 - DNA, 84 bp, Synthetic construct
  source 1..84: mol_type = other DNA
agataatggcactcgtggctgtggtggcttcggcagcggcttcagcagatcggcggcatcagcggtagcaccagcactgccacc

SEQ ID NO: 272 - DNA, 59 bp, Synthetic construct
  source 1..59: mol_type = other DNA
agataatcctgctttctgcattcttctctccacatccctctctgtacttacaggccacc

SEQ ID NO: 273 - DNA, 139 bp, Synthetic construct
  source 1..139: mol_type = other DNA
agataatagctgcgcctggctgcgcacagagctccctcccaggcccgcgaacttggccattcagccgccgctgtccccgctgcgcgccctcgcgcctctgcctgagaagccaggcgctgttcccccaccccaggccacc

SEQ ID NO: 274 - DNA, 82 bp, Synthetic construct
  source 1..82: mol_type = other DNA
agataataactcgccgctgccggcctgacctcgctcccagccctgctgcccagattctaggctccaagctcaggacgccacc

SEQ ID NO: 275 - DNA, 378 bp, Synthetic construct
  source 1..378: mol_type = other DNA
agataatacacggtaagagggagagcccaggtttgaacaagtctatctgattccaaagcctaaggtccttcttagcactcttcagcctgtttagataactggggaaggggagggtgggtgctctcccagaagcatattcattcagtaaactcagagcatctattttgtgccactctgcacggtactgaacgtacaaagatgatgaagtggtctctgtgggccttgggagcccatgttcaaggcaggaccattggaaaacagcatccctcatgcttgacccggatggggcaggacattgatgaatcgtgatggagggggtgactatgggatgcttcaaaaaaaaggcttcctggaggagatgacatttaactggccacc

SEQ ID NO: 276 - DNA, 71 bp, Synthetic construct
  source 1..71: mol_type = other DNA
agataatatctttgggtggtggagtgcaaaggaggcgacctgcaacagaggagtcccggtcaccagccacc

SEQ ID NO: 277 - DNA, 45 bp, Synthetic construct
  source 1..45: mol_type = other DNA
agataattggcgggcattcctgaagctgacagcattcgggccacc

SEQ ID NO: 278 - DNA, 86 bp, Synthetic construct
  source 1..86: mol_type = other DNA
agataatgcaggcggagtgcctgagtgccctctcactcaaccctctctaccctttgtctgcagaggcggccgctgacagcgccacc

SEQ ID NO: 279 - DNA, 53 bp, Synthetic construct
  source 1..53: mol_type = other DNA
aggaaaaacgcccctgacacaagtgtgttcactaccaacctcccacagccacc

SEQ ID NO: 280 - DNA, 255 bp, Synthetic construct
  source 1..255: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaacccca

SEQ ID NO: 281 - DNA, 133 bp, Synthetic construct
  source 1..133: mol_type = other DNA
gctcgctttcttgctgtccaatttctattaaaggttcctttgttccctaagtccaactactaaactgggggatattatgaagggccttgagcatctggattctgcctaataaaaaacatttattttcattgca

SEQ ID NO: 282 - DNA, 120 bp, Synthetic construct
  source 1..120: mol_type = other DNA
gtgtgtggaggacaccctgaaccccccgctttcaaacaagttttcaaattgtttgaggtcaggatttctcaaactgattcctttctttgcatatgagtatttgaaaataaatattttccc

SEQ ID NO: 283 - DNA, 83 bp, Synthetic construct
  source 1..83: mol_type = other DNA
ttgaggatagtcaggagcgtcaatgtgcttgtacatagagtgctgtagctgtgtgttccaataaattattttgtagggaaagt

SEQ ID NO: 284 - DNA, 130 bp, Synthetic construct
  source 1..130: mol_type = other DNA
cctcgccccggacctgccctcccgccaggtgcacccacctgcaataaatgcagcgaagccgggacctcctcgcgggccaccacgcctcccgcgtccaccaccgtggaccaataaatcgtgcgaagggccc

SEQ ID NO: 285 - DNA, 77 bp, Synthetic construct
  source 1..77: mol_type = other DNA
acatctcttgccatcacctagctgcctgcacctgcccttcagggagatgggggtcattaaaggaaactgaacattga

SEQ ID NO: 286 - DNA, 113 bp, Synthetic construct
  source 1..113: mol_type = other DNA
taagctggagcctcggtggccatgcttcttgccccttgggcctccccccagcccctcctccccttcctgcacccgtacccccgtggtctttgaataaagtctgagtgggcggc

SEQ ID NO: 287 - DNA, 110 bp, Synthetic construct
  source 1..110: mol_type = other DNA
gctggagcctcggtggcctagcttcttgccccttgggcctccccccagcccctcctccccttcctgcacccgtacccccgtggtctttgaataaagtctgagtgggcggc

SEQ ID NO: 288 - DNA, 278 bp, Synthetic construct
  source 1..278: mol_type = other DNA
accgaatatctccaatgctctcccctttcatatctagcgtggtccggaatggaaaagtgggactggatgctacaaactgtttgaggataactgacttaaaatctggctgcacatcattgactcctgggcccaactgtgaccgatttaaactgcacataccatatgctggagagacattaaagtgggatatcattttcaatgcccaatacccagaactgcctcccgattttatctttggagaagatgctgaattcctgccagacccctcagctttgcag

SEQ ID NO: 289 - DNA, 223 bp, Synthetic construct
  source 1..223: mol_type = other DNA
ggatagaggggaggccttattgtatcggttttatattacctgttctgcttcaccaggagatcatgctgctgtgatactgagttttctaaacagcataaggaagacttgctcccctgtcctatgaaagagaatagttttggaggggagaagtgggacaaaaaagatgcagttttcctttgtattgggaaatgtgaaaataaaattgtcaactctttcagttaaa

SEQ ID NO: 290 - DNA, 288 bp, Synthetic construct
  source 1..288: mol_type = other DNA
acacactccacctccagcacgcgacttctcaggacgacgaatcttctcaatgggggggcggctgagctccagccaccccgcagtcactttctttgtaacaacttccgttgctgccatcgtaaactgacacagtgtttataacgtgtacatacattaacttattacctcattttgttatttttcgaaacaaagccctgtggaagaaaatggaaaacttgaagaagcattaaagtcattctgttaagctgcgtaaatggtctttgaataaagcctgagtaggaagtctag

SEQ ID NO: 291 - DNA, 378 bp, Synthetic construct
  source 1..378: mol_type = other DNA
catcacatttaaaagcatctcagcctaccatgagaataagagaaagaaaatgaagatcaaaagtctattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaatggtctttgaataaagcctgagtaggaagtctag

SEQ ID NO: 292 - DNA, 142 bp, Synthetic construct
  source 1..142: mol_type = other DNA
tgataatagtccataaagtaggaaacactacagctggagcctcggtggccatgcttcttgccccttgggcctccccccagcccctcctccccttcctgcacccgtacccccgtggtctttgaataaagtctgagtgggcggc

SEQ ID NO: 293 - DNA, 142 bp, Synthetic construct
  source 1..142: mol_type = other DNA
tgataataggctggagcctcggtggctccataaagtaggaaacactacacatgcttcttgccccttgggcctccccccagcccctcctccccttcctgcacccgtacccccgtggtctttgaataaagtctgagtgggcggc

SEQ ID NO: 294 - DNA, 141 bp, Synthetic construct
  source 1..141: mol_type = other DNA
tgataataggctggagcctcggtggccatgcttcttgccccttgggcctccccccagtccataaagtaggaaacactacacccctcctccccttcctgcacccgtacccccgtggtctttgaaaaagtctgagtgggcggc

SEQ ID NO: 295 - DNA, 142 bp, Synthetic construct
  source 1..142: mol_type = other DNA
tgataataggctggagcctcggtggccatgcttcttgccccttgggcctccccccagcccctcctccccttctccataaagtaggaaacactacactgcacccgtacccccgtggtctttgaataaagtctgagtgggcggc

SEQ ID NO: 296 - DNA, 142 bp, Synthetic construct
  source 1..142: mol_type = other DNA
tgataataggctggagcctcggtggccatgcttcttgccccttgggcctccccccagcccctcctccccttcctgcacccgtaccccctccataaagtaggaaacactacagtggtctttgaataaagtctgagtgggcggc

SEQ ID NO: 297 - DNA, 142 bp, Synthetic construct
  source 1..142: mol_type = other DNA
tgataataggctggagcctcggtggccatgcttcttgccccttgggcctccccccagcccctcctccccttcctgcacccgtacccccgtggtctttgaataaagttccataaagtaggaaacactacactgagtgggcggc

SEQ ID NO: 298 - DNA, 164 bp, Synthetic construct
  source 1..164: mol_type = other DNA
tgataatagtccataaagtaggaaacactacagctggagcctcggtggccatgcttcttgccccttgggcctccccccagcccctcctccccttcctgcacccgtaccccccgcattattactcacggtacgagtggtctttgaataaagtctgagtgggcggc

SEQ ID NO: 299 - DNA, 164 bp, Synthetic construct
  source 1..164: mol_type = other DNA
tgataatagtccataaagtaggaaacactacagctggagcctcggtggcctagcttcttgccccttgggcctccccccagcccctcctccccttcctgcacccgtaccccccgcattattactcacggtacgagtggtctttgaataaagtctgagtgggcggc

SEQ ID NO: 300 - DNA, 141 bp, Synthetic construct
  source 1..141: mol_type = other DNA
tgataaaggctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 301 - DNA, 142 bp, Synthetic construct
  source 1..142: mol_type = other DNA
tgataataggctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 302 - DNA, 118 bp, Synthetic construct
  source 1..118: mol_type = other DNA
tgataataggctggagcctcggtggccatgcttcttgccccttgggcctcccccagcccctcctccccttcctgcacccgtacccccgtggtctttgaataaagtctgagtgggcggc

SEQ ID NO: 303 - DNA, 163 bp, Synthetic construct
  source 1..163: mol_type = other DNA
tgataataggctggagcctcggtggccatgcttcttgccccttgggcccaaacaccattgtcacactccatccccccagcccctcctccccttcctccataaagtaggaaacactacatgcacccgtacccccgtggtcttgaataaagtctgagtgggcggc

SEQ ID NO: 304 - DNA, 370 bp, Synthetic construct
  source 1..370: mol_type = other DNA
gcgcctgcccacctgccaccgactgctggaacccagccagtgggagggcctggcccaccagagtcctgctccctcactcctcgccccgccccctgtcccagagtcccacctgggggctctctccacccttctcagagttccagtttcaaccagagttccaaccaatgggctccatcctctggattctggccaatgaaatatctccctggcagggtcctcttcttttcccagagctccaccccaaccaggagctctagttaatggagagctcccagcacactcggagcttgtgctttgtctccacgcaaagcgataaataaaagcattggtggcctttggtctttgaataaagcctgagtaggaagtctag

SEQ ID NO: 305 - DNA, 566 bp, Synthetic construct
  source 1..566: mol_type = other DNA
gcccctgccgctcccacccccacccatctgggccccgggttcaagagagagcggggtctgatctcgtgtagccatatagagtttgcttctgagtgtctgctttgtttagtagaggtgggcaggaggagctgaggggctggggctggggtgttgaagttggctttgcatgcccagcgatgcgcctccctgtgggatgtcatcaccctgggaaccgggagtggcccttggctcactgtgttctgcatggtttggatctgaattaattgtcctttcttctaaatcccaaccgaacttcttccaacctccaaactggctgtaaccccaaatccaagccattaactacacctgacagtagcaattgtctgattaatcactggccccttgaagacagcagaatgtccctttgcaatgaggaggagatctgggctgggcgggccagctggggaagcatttgactatctggaacttgtgtgtgcctcctcaggtatggcagtgactcacctggttttaaaaaacaacctgcaacatctcatggtctttgaataaagcctgagtaggaagtctag

SEQ ID NO: 306 - DNA, 378 bp, Synthetic construct
  source 1..378: mol_type = other DNA
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaatggtctttgaataaagcctgagtaggaagtctag

SEQ ID NO: 307 - DNA, 117 bp, Synthetic construct
  source 1..117: mol_type = other DNA
gctgccttctgcggggcttgccttctggccatgcccttcttctctcccttgcacctgtacctcttggtctttgaataaagcctgagtaggaaggcggccgctcgagcatgcatctag

SEQ ID NO: 308 - DNA, 116 bp, Synthetic construct
  source 1..116: mol_type = other DNA
taataggctggagcctcggtggccatgcttcttgccccttgggcctccccccagcccctcctccccttcctgcacccgtacccccgtggtctttgaataaagtctgagtgggcggc

SEQ ID NO: 309 - DNA, 118 bp, Synthetic construct
  source 1..118: mol_type = other DNA
tgataataggctggagcctcggtggccatgcttcttgccccttgggcctccccccagcccctcctccccttcctgcacccgtaccccctggtctttgaataaagtctgagtgggcggc

SEQ ID NO: 310 - DNA, 119 bp, Synthetic construct
  source 1..119: mol_type = other DNA
tgataataggctggagcctcggtggccatgcttcttgccccttgggcctccccccagcccctcctccccttcctgcacccgtacccccgtggtctttgaataaagtctgagtgggcggc

SEQ ID NO: 311 - DNA, 119 bp, Synthetic construct
  source 1..119: mol_type = other DNA
tgataataggctggagcctcggtggcctagcttcttgccccttgggcctccccccagcccctcctccccttcctgcacccgtacccccgtggtctttgaataaagtctgagtgggcggc

SEQ ID NO: 312 - DNA, 96 bp, Synthetic construct
  source 1..96: mol_type = other DNA
agcgctgccttctgcggggcttgccttctggccatgcccttcttctctcccttgcacctgtacctcttggtctttgaataaagcctgagtaggaag

SEQ ID NO: 313 - DNA, 102 bp, Synthetic construct
  source 1..102: mol_type = other DNA
tgataataggctgccttctgcggggcttgccttctggccatgcccttcttctctcccttgcacctgtacctcttggtctttgaataaagcctgagtaggaag

SEQ ID NO: 314 - DNA, 169 bp, Synthetic construct
  source 1..169: mol_type = other DNA
ggccaggccccttccctctgcccccgggtgcttgaagtctagccccatcctggtccaatgcgctcttggtagcctcctttcccagctgcccgcccgccgccatgccgcccttactgcccctgcgcctgtgccggctgtggccccgcaaccctccctcccggctcctcgg

SEQ ID NO: 315 - DNA, 143 bp, Synthetic construct
  source 1..143: mol_type = other DNA
tgcccgtcctcaccaagactgactgcctgctgctttgctactgcccgggcccatgagactgacttcccactgctctgcctgcctctccccactgcactggcacagccccgccttgccgctgctgatccattgccggtgtgacc

SEQ ID NO: 316 - DNA, 141 bp, Synthetic construct
  source 1..141: mol_type = other DNA
tgctgcctgcttcttgctccagcaccatggaatgcctgcgcagtttaccctgcctcctgccccgcgcgatgagacttccccggcggacgctgtgtgccctggccttggacgtgacctctgtgggtcctcccgttgctgcct

SEQ ID NO: 317 - DNA, 149 bp, Synthetic construct
  source 1..149: mol_type = other DNA
ttccagccagacacccgccccccggccctggctaagaagttgcttcctgttgccagcatgacctaccctcgcctctttgatgccatccgctgccacctccttttgctcctggaccctttagcctctctgcccttccactctctgacccc

SEQ ID NO: 318 - DNA, 155 bp, Synthetic construct
  source 1..155: mol_type = other DNA
gccttggctcctccaggaaggctcaggagccctacctccctgccattatagctgctccccgccagaagcctgtgccaactctctgcattccctgatctccatccctgtggctgtcacccttggtcacctccgtgctgtcactgccatctcccccc

SEQ ID NO: 319 - DNA, 190 bp, Synthetic construct
  source 1..190: mol_type = other DNA
ctgacagcgtgggcaacgcctgccgcctgctctgaggcccgatccagtgggcaggccaaggcctgctgggcccccgcggacccaggtgctctgggtcacggtccctgtccccgcacccccgcttctgtctgccccattgtggctcctcaggctctctcccctgctctcccacctctacctccacccccac

SEQ ID NO: 320 - DNA, 233 bp, Synthetic construct
  source 1..233: mol_type = other DNA
ctttgcaggatgaaacacttccccgcttggctctcattcttccacaagagagacctttctccggacctggttgctactggttcagcaactctgcagaaaatgtcctcccctgtggctgcctcagctcatgcctttggcctgaagtcccagcattgatggcagcccctcatcttccaagttttgtgctcccctttacctaacgcttcctgcctcccatgcatctgtactcctcc

SEQ ID NO: 321 - DNA, 169 bp, Synthetic construct
  source 1..169: mol_type = other DNA
ccttgagcttggagtcctcctcctccagtaggcgctgcgtctccttcttacgttccagctggtcgaggcgccgcttctccttctcctccttgcgctgctccttcctcatgacgtgtttgtcgtcgtccttccagtaggcatcctccagctccttctgcttcttggcatc

SEQ ID NO: 322 - DNA, 276 bp, Synthetic construct
  source 1..276: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaaccctcgagctcgctttcttgctgtccaatttctattaaaggttcctttgttccctaagtccaactactaaactgggggatattatgaagggccttgagcatctggattctgcctaataaaaaacatttattttcattgcgaa

SEQ ID NO: 323 - DNA, 597 bp, Synthetic construct
  source 1..597: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcccctcgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 324 - DNA, 286 bp, Synthetic construct
  source 1..286: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaaccctcgaaggcaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 325 - DNA, 277 bp, Synthetic construct
  source 1..277: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaaccctcgactggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 326 - DNA, 288 bp, Synthetic construct
  source 1..288: mol_type = other DNA
aggcaagcacgcagcaatgcagctcaaaacgcttagcctagccacacccccacgggaaacagcagtgattaacctttagcaataaacgaaagtttaactaagctatactaaccccagggttggtcaatttcgtgccagccacaccccctcgactggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 327 - DNA, 261 bp, Synthetic construct
  source 1..261: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaaccctcgagtgtgtggaggacaccctgaaccccccgctttcaaacaagttttcaaattgtttgaggtcaggatttctcaaactgattcctttctttgcatatgagtatttgaaaataaatattttccc

SEQ ID NO: 328 - DNA, 247 bp, Synthetic construct
  source 1..247: mol_type = other DNA
gtgtgtggaggacaccctgaaccccccgctttcaaacaagttttcaaattgtttgaggtcaggatttctcaaactgattcctttctttgcatatgagtatttgaaaataaatattttcccccctcgagtgtgtggaggacaccctgaaccccccgctttcaaacaagttttcaaattgtttgaggtcaggatttctcaaactgattcctttctttgcatatgagtatttgaaaataaatattttccc

SEQ ID NO: 329 - DNA, 60 bp, Synthetic construct
  source 1..60: mol_type = other DNA
cgtctgcataacttttattatttcttttattaatcaacaaaattttgtttttaacatttc

SEQ ID NO: 330 - DNA, 201 bp, Synthetic construct
  source 1..201: mol_type = other DNA
gtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccttcggggtactcttaacgctgacactcgaaagagcagttcggcaaccc

SEQ ID NO: 331 - DNA, 436 bp, Synthetic construct
  source 1..436: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaaccctcgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 332 - DNA, 273 bp, Synthetic construct
  source 1..273: mol_type = other DNA
agctcgctttcttgctccccaatttctattattccttggaatgaagggaaagtccaactactaaactgtcccttattttgttccaggaagagctagacctatctgccttaaacgagacacgtttattcaccgcaccctcgagctcgctttcttgctgtccaatttctattaaaggttcctttgttccctaagtccaactactaaactgggggatattatgaagggccttgagcatctggattctgcctaataaaaaacatttattttcattgc

SEQ ID NO: 333 - DNA, 71 bp, Synthetic construct
  source 1..71: mol_type = other DNA
aggcaaaaatcaaaatcaatcatcatcacaacatcaacaatcaatcatcaacacatcatcaagacgccacc

SEQ ID NO: 334 - DNA, 113 bp, Synthetic construct
  source 1..113: mol_type = other DNA
aatgtcagaaatattaacaaaaattctcagcattaaagaaaacattgattttcctttcctgactataaaactaattgtgcattatagaaaagtttaaatcacagaatggtatt

SEQ ID NO: 335 - RNA, 58 nt, Synthetic construct
  source 1..58: mol_type = other RNA
aggaaataagacgaggaatagagagtaagaagaaatataaggccagtgcgccgccacc

SEQ ID NO: 336 - RNA, 57 nt, Synthetic construct
  source 1..57: mol_type = other RNA
aggaaataagagaggataagacgactaaggagacatacagaataagaggcagccacc

SEQ ID NO: 337 - RNA, 59 nt, Synthetic construct
  source 1..59: mol_type = other RNA
aggaaataagaaagaagacagaagaagacagaagaagaaccagagaaggacaagccacc

SEQ ID NO: 338 - RNA, 50 nt, Synthetic construct
  source 1..50: mol_type = other RNA
agaagagaacctcgtcgagtcctggtagtagtaatcctagaggagccacc

SEQ ID NO: 339 - RNA, 50 nt, Synthetic construct
  source 1..50: mol_type = other RNA
aggatataaccaattttgtgaattacgagctaaaacgtcttcgtgccacc

SEQ ID NO: 340 - RNA, 50 nt, Synthetic construct
  source 1..50: mol_type = other RNA
aggagggtaattcgcttagcgatagtactatcgaagcgtacagagccacc

SEQ ID NO: 341 - RNA, 50 nt, Synthetic construct
  source 1..50: mol_type = other RNA
aggaggactgcgcgaacctgcatagtgatcataaggtcatgatagccacc

SEQ ID NO: 342 - RNA, 50 nt, Synthetic construct
  source 1..50: mol_type = other RNA
aggtatctaattcgacaattggtctacaaattatcctaacgcctgccacc

SEQ ID NO: 343 - RNA, 92 nt, Synthetic construct
  source 1..92: mol_type = other RNA
aggtccccataagatactttaacaagatcactaggtttagacgctgtgcagtcgtcccggactactacagccgcgccgcagtcttcgccacc

SEQ ID NO: 344 - RNA, 53 nt, Synthetic construct
  source 1..53: mol_type = other RNA
aggacatttgctcctgacacaaccgtgagcactagcaccttcacactgccacc

SEQ ID NO: 345 - RNA, 53 nt, Synthetic construct
  source 1..53: mol_type = other RNA
aggaaacttgctactaccagcaccatgctcgtcaacatcctcaactagccacc

SEQ ID NO: 346 - RNA, 53 nt, Synthetic construct
  source 1..53: mol_type = other RNA
aggacattagcttctgacacaactgccttcgctaccaacatcacaccgccacc

SEQ ID NO: 347 - RNA, 53 nt, Synthetic construct
  source 1..53: mol_type = other RNA
aggacatttgcttctgacaccactgtattcgctggcatcctcaacttgccacc

SEQ ID NO: 348 - RNA, 53 nt, Synthetic construct
  source 1..53: mol_type = other RNA
aggacatttacttctgacccaactgagttgactagcgacctgaactagccacc

SEQ ID NO: 349 - RNA, 53 nt, Synthetic construct
  source 1..53: mol_type = other RNA
aggacatttgcttctgtcacaactttgttcactagcagtatcgcccagccacc

SEQ ID NO: 350 - RNA, 53 nt, Synthetic construct
  source 1..53: mol_type = other RNA
aggacatttccttctgacacaactgtattcaaccgtaagctcaaaaagccacc

SEQ ID NO: 351 - RNA, 53 nt, Synthetic construct
  source 1..53: mol_type = other RNA
aggacattagtttctgacacaactgtcaggactagcaacatcaaccagccacc

SEQ ID NO: 352 - RNA, 53 nt, Synthetic construct
  source 1..53: mol_type = other RNA
aggacattggcttctgacgcaactgggaccactagcaaccgcaactagccacc

SEQ ID NO: 353 - RNA, 57 nt, Synthetic construct
  source 1..57: mol_type = other RNA
aggaaataagaacataacacgacacatataacacactcaacgacaccagaagccacc

SEQ ID NO: 354 - RNA, 50 nt, Synthetic construct
  source 1..50: mol_type = other RNA
agagagagagagagagagagagagagagagagagagagagagaggccacc

SEQ ID NO: 355 - RNA, 50 nt, Synthetic construct
  source 1..50: mol_type = other RNA
aggatggggcctaacaaaatggcggggccggggccccgggccgggccacc

SEQ ID NO: 356 - RNA, 50 nt, Synthetic construct
  source 1..50: mol_type = other RNA
aggaggactgcgcgaacctgcatagtgatcataaggtcacgatagccacc

SEQ ID NO: 357 - RNA, 50 nt, Synthetic construct
  source 1..50: mol_type = other RNA
aggaggactgggcgaacctgcatagtgatcataaggtcatcatagccacc

SEQ ID NO: 358 - RNA, 58 nt, Synthetic construct
  source 1..58: mol_type = other RNA
aggaaataaggaggactgggcgaacctgcatagtgatcataaggtcatcatagccacc

SEQ ID NO: 359 - RNA, 53 nt, Synthetic construct
  source 1..53: mol_type = other RNA
aggacatttgcttctgacacaactgtgttcactagcaacctcaaacagccacc

SEQ ID NO: 360 - RNA, 53 nt, Synthetic construct
  source 1..53: mol_type = other RNA
agaccataaataaacaacacaaacaacaaacaacccaaataatacccgccacc

SEQ ID NO: 361 - RNA, 57 nt, Synthetic construct
  source 1..57: mol_type = other RNA
aggaaataagacccgggcccgggcccgggcccgggcccgggcccgggccctgccacc

SEQ ID NO: 362 - RNA, 53 nt, Synthetic construct
  source 1..53: mol_type = other RNA
aggtggagagagtgtgtgagatgagtcgcggaatgagccgaccttctgccacc

SEQ ID NO: 363 - RNA, 53 nt, Synthetic construct
  source 1..53: mol_type = other RNA
aggtgagagagtgtagagtgactgaaattgccaaacgtaccgtaatggccacc

SEQ ID NO: 364 - RNA, 53 nt, Synthetic construct
  source 1..53: mol_type = other RNA
aggtgagagagtttaacttgatgaccatgtctaggcataatatgcctgccacc

SEQ ID NO: 365 - RNA, 53 nt, Synthetic construct
  source 1..53: mol_type = other RNA
aggtgggaagagtgtttcagagagagatgttcactaacttgtcaagggccacc

SEQ ID NO: 366 - RNA, 53 nt, Synthetic construct
  source 1..53: mol_type = other RNA
agggggggtgccaggaacttgctactaccagcaccatgctgtaccgcgccacc

SEQ ID NO: 367 - RNA, 53 nt, Synthetic construct
  source 1..53: mol_type = other RNA
aggagcgggcattgcccaggaacttgctactaccagcaccatcctgcgccacc

SEQ ID NO: 368 - RNA, 87 nt, Synthetic construct
  source 1..87: mol_type = other RNA
aggaccacacaaaccgcgtccgccacgctagcggggatcctgtcctttccagatccggaatccgtacacattcaacgcaaggccacc

SEQ ID NO: 369 - RNA, 87 nt, Synthetic construct
  source 1..87: mol_type = other RNA
aggacagacgagaccgcgtccgccccgcgagcacagaccctttcctattccgatctgcatcccttccatagtaaaaggggggccacc

SEQ ID NO: 370 - RNA, 87 nt, Synthetic construct
  source 1..87: mol_type = other RNA
aggacccacgagtgcgcgtgcaccccgcgagcacagagtctcgctgttaactatccgcagcacgtcgacatcactcacctagccacc

SEQ ID NO: 371 - RNA, 87 nt, Synthetic construct
  source 1..87: mol_type = other RNA
aggactcccgagaacgtgtccgcccctcgagcacagagcctcgaccttgccgatccgcctgtcgtccacaccagccgcctagccacc

SEQ ID NO: 372 - RNA, 61 nt, Synthetic construct
  source 1..61: mol_type = other RNA
aggaaataagagaaagaggataagacgactaaggagacatacagaataagaggcagccacc

SEQ ID NO: 373 - RNA, 61 nt, Synthetic construct
  source 1..61: mol_type = other RNA
aggaaataagagaggataagacgactaaggagacatacagaataagaaacaggcagccacc

SEQ ID NO: 374 - RNA, 50 nt, Synthetic construct
  source 1..50: mol_type = other RNA
aggaggactggtcgaacctgcatagtgatcataaggtcagcatagccacc

SEQ ID NO: 375 - RNA, 50 nt, Synthetic construct
  source 1..50: mol_type = other RNA
aggaggactggccgaacctgcatagtgatcataaggtcagcatagccacc

SEQ ID NO: 376 - RNA, 57 nt, Synthetic construct
  source 1..57: mol_type = other RNA
aggaaataagagagaaaagaagagtaagaagaaatataagaccccggcgccgccacc

SEQ ID NO: 377 - RNA, 86 nt, Synthetic construct
  source 1..86: mol_type = other RNA
aggaccgccgagaccgcgtccgccccgcgagcacagagcctcgcctttgccgatccgccgcccgtccacacccgccgccagccacc

SEQ ID NO: 378 - RNA, 53 nt, Synthetic construct
  source 1..53: mol_type = other RNA
aggtgtgagagtttaacttgaagaccatctctagggataatatccctgccacc

SEQ ID NO: 379 - RNA, 53 nt, Synthetic construct
  source 1..53: mol_type = other RNA
agattggagaactttgtttgattatcagaatccttagtagaattggggccacc

SEQ ID NO: 380 - RNA, 53 nt, Synthetic construct
  source 1..53: mol_type = other RNA
aggtgtcagagtttaacttgaagactatttctagggataatatccctgccacc

SEQ ID NO: 381 - RNA, 53 nt, Synthetic construct
  source 1..53: mol_type = other RNA
agagggccggacacagtcccacgctcgcccctccccgtcccgcgggggccacc

SEQ ID NO: 382 - RNA, 53 nt, Synthetic construct
  source 1..53: mol_type = other RNA
aggtgtgagagtttaacttgaagaccatcactcgcgataatatcgcggccacc

SEQ ID NO: 383 - RNA, 53 nt, Synthetic construct
  source 1..53: mol_type = other RNA
aggtcagagagctaagcttaattaccatctctacgactacaagtcgtgccacc

SEQ ID NO: 384 - RNA, 53 nt, Synthetic construct
  source 1..53: mol_type = other RNA
aggtgtgtgagtttaacttgaagaccgtatctagggataatatccctgccacc

SEQ ID NO: 385 - RNA, 53 nt, Synthetic construct
  source 1..53: mol_type = other RNA
agtacaaattccgaaggaacgaatacagaattcaagttattaacttggccacc

SEQ ID NO: 386 - RNA, 53 nt, Synthetic construct
  source 1..53: mol_type = other RNA
aggtgtaagaggttaacttgaagaccatatctagggataatatcgctgccacc

SEQ ID NO: 387 - RNA, 53 nt, Synthetic construct
  source 1..53: mol_type = other RNA
agttttaaaagtttggctacaagagcattagcagcgaagagatcgctgccacc

SEQ ID NO: 388 - RNA, 53 nt, Synthetic construct
  source 1..53: mol_type = other RNA
agctgagagagtttaacttgacggccataactaggattatagctcctgccacc

SEQ ID NO: 389 - RNA, 53 nt, Synthetic construct
  source 1..53: mol_type = other RNA
aggtcttcgaacttatctcaataaccattggccgtattagagtagtagccacc

SEQ ID NO: 390 - RNA, 53 nt, Synthetic construct
  source 1..53: mol_type = other RNA
aggtgagagaggtgagcgcgaaggccaggtcttggcataaggtgcctgccacc

SEQ ID NO: 391 - RNA, 53 nt, Synthetic construct
  source 1..53: mol_type = other RNA
agttcagtataacgcacttggctccaggaacttttagatatagtagagccacc

SEQ ID NO: 392 - RNA, 53 nt, Synthetic construct
  source 1..53: mol_type = other RNA
agcttggagaacttttactagagagtagaaacgttaatagaattggggccacc

SEQ ID NO: 393 - RNA, 53 nt, Synthetic construct
  source 1..53: mol_type = other RNA
aggtcctagagctggactggattaccagtactagtcaatcaatttctgccacc

SEQ ID NO: 394 - RNA, 53 nt, Synthetic construct
  source 1..53: mol_type = other RNA
agttctgtgagtataaatttaggctcattactaggcaccataatccggccacc

SEQ ID NO: 395 - RNA, 53 nt, Synthetic construct
  source 1..53: mol_type = other RNA
aggtgcgggagtggggcgtgctgaccaggtctaggcttagtattcctgccacc

SEQ ID NO: 396 - RNA, 53 nt, Synthetic construct
  source 1..53: mol_type = other RNA
aggtgcgagagtttaacttggagaccatctctagggagaatctccctgccacc

SEQ ID NO: 397 - RNA, 53 nt, Synthetic construct
  source 1..53: mol_type = other RNA
agaataaactagtattcttctggtccccacagactcagagagaacccgccacc

SEQ ID NO: 398 - skipped

SEQ ID NO: 399 - RNA, 61 nt, Synthetic construct
  source 1..61: mol_type = other RNA
agtcctccccatcctctccctctgtccctctgtccctctgaccctgcactgtcccgccacc

SEQ ID NO: 400 - RNA, 39 nt, Synthetic construct
  source 1..39: mol_type = other RNA
agactcttctggtccccacagactcagagagaagccacc

SEQ ID NO: 401 - RNA, 55 nt, Synthetic construct
  source 1..55: mol_type = other RNA
agacgcccctctttctccccaggccgaagcctcgggacggccctggaaggccacc

SEQ ID NO: 402 - RNA, 56 nt, Synthetic construct
  source 1..56: mol_type = other RNA
agaataaactcactatttgttttcgcgcccagttgcaaaaagtgtcgaacgccacc

SEQ ID NO: 403 - RNA, 50 nt, Synthetic construct
  source 1..50: mol_type = other RNA
agctcgtcagctagctagacttctttgctaggacaaccatgttagccacc

SEQ ID NO: 404 - RNA, 50 nt, Synthetic construct
  source 1..50: mol_type = other RNA
agaactataacaatcaaagtccctgtgtccgcgaagccatgagagccacc

SEQ ID NO: 405 - RNA, 50 nt, Synthetic construct
  source 1..50: mol_type = other RNA
aggctagcagcatagttagtctacaatattacactaacaagagagccacc

SEQ ID NO: 406 - RNA, 50 nt, Synthetic construct
  source 1..50: mol_type = other RNA
agtagcataagtggtccaggactcttagttcgcaaataatgcgcgccacc

SEQ ID NO: 407 - RNA, 50 nt, Synthetic construct
  source 1..50: mol_type = other RNA
agacacttagcttacgtcatttcctggcaattagtaactgaatagccacc

SEQ ID NO: 408 - RNA, 50 nt, Synthetic construct
  source 1..50: mol_type = other RNA
agcaattcacactttccattgcggaagaattctttagcatccaagccacc

SEQ ID NO: 409 - RNA, 50 nt, Synthetic construct
  source 1..50: mol_type = other RNA
agtatccagattagcaaaacccaaacaatcttgtaagaatccaagccacc

SEQ ID NO: 410 - RNA, 50 nt, Synthetic construct
  source 1..50: mol_type = other RNA
agtggaccacgcagaagattgttgagactattatgctagtccgtgccacc

SEQ ID NO: 411 - RNA, 50 nt, Synthetic construct
  source 1..50: mol_type = other RNA
agcctactcgtctttagagagagatacacaatacttatatgagagccacc

SEQ ID NO: 412 - RNA, 50 nt, Synthetic construct
  source 1..50: mol_type = other RNA
agtgacaaaagtagccaaattctcctagcctcaaccaagaatcggccacc

SEQ ID NO: 413 - RNA, 50 nt, Synthetic construct
  source 1..50: mol_type = other RNA
agtgtctatcccattcattccgtgatctactacttagcaaccgtgccacc

SEQ ID NO: 414 - RNA, 50 nt, Synthetic construct
  source 1..50: mol_type = other RNA
agtgtccatctttggcaagttgctctagcatgataattttgagggccacc

SEQ ID NO: 415 - RNA, 50 nt, Synthetic construct
  source 1..50: mol_type = other RNA
agtctgaaactaatccagttggtgaggacattacaagagtctcggccacc

SEQ ID NO: 416 - RNA, 50 nt, Synthetic construct
  source 1..50: mol_type = other RNA
agacgtctaactgttccctggactaatatacaattgaaatgaaagccacc

SEQ ID NO: 417 - RNA, 50 nt, Synthetic construct
  source 1..50: mol_type = other RNA
aggtggttcttctgcaaattagcgaccaaataatgagaatcagcgccacc

SEQ ID NO: 418 - RNA, 50 nt, Synthetic construct
  source 1..50: mol_type = other RNA
agatcaaatactaacgaattcgtgagcgcttaatcgcgacgtttgccacc

SEQ ID NO: 419 - RNA, 50 nt, Synthetic construct
  source 1..50: mol_type = other RNA
agaaacaccagtaggcaacattttgtagcaactctgttaagagagccacc

SEQ ID NO: 420 - RNA, 50 nt, Synthetic construct
  source 1..50: mol_type = other RNA
agtatatagaatatctagttgatagaagaccaatgagtacagctgccacc

SEQ ID NO: 421 - RNA, 50 nt, Synthetic construct
  source 1..50: mol_type = other RNA
agacaaattgctaccttagaaaccgagagaagcgtatcatcagagccacc

SEQ ID NO: 422 - RNA, 50 nt, Synthetic construct
  source 1..50: mol_type = other RNA
agtcgctaactgaaccacttaccgataattcatatataaacgctgccacc

SEQ ID NO: 423 - RNA, 50 nt, Synthetic construct
  source 1..50: mol_type = other RNA
agtccatagcgctaattagctatttacattatcctcacagcaacgccacc

SEQ ID NO: 424 - RNA, 50 nt, Synthetic construct
  source 1..50: mol_type = other RNA
aggccgtcacctaaagaaatacttgtgatcatttatcaataattgccacc

SEQ ID NO: 425 - RNA, 50 nt, Synthetic construct
  source 1..50: mol_type = other RNA
agtaaataaccgtgttcggtagctcctgtggtaaggactacatagccacc

SEQ ID NO: 426 - RNA, 50 nt, Synthetic construct
  source 1..50: mol_type = other RNA
agtatcagagtgaaccctgagatactaatatcctgagagtttaagccacc

SEQ ID NO: 427 - RNA, 50 nt, Synthetic construct
  source 1..50: mol_type = other RNA
aggaacaaccccgagaacaaggttttcttatcatccggtttcgagccacc

SEQ ID NO: 428 - RNA, 50 nt, Synthetic construct
  source 1..50: mol_type = other RNA
agctaatctgcttatctgtttctcgatataattaaagcattacagccacc

SEQ ID NO: 429 - RNA, 50 nt, Synthetic construct
  source 1..50: mol_type = other RNA
agtaactaactgattggcttagctctgtaagcaacccaattctggccacc

SEQ ID NO: 430 - RNA, 50 nt, Synthetic construct
  source 1..50: mol_type = other RNA
agtctcttcactgctttcattctttataaagttccaaaatgggtgccacc

SEQ ID NO: 431 - RNA, 50 nt, Synthetic construct
  source 1..50: mol_type = other RNA
agaatatatagcctctaattgttggccattttaacctctgccgcgccacc

SEQ ID NO: 432 - RNA, 50 nt, Synthetic construct
  source 1..50: mol_type = other RNA
aggcggtccaacatcttgtgactttagcttgcaaagttatgacagccacc

SEQ ID NO: 433 - RNA, 50 nt, Synthetic construct
  source 1..50: mol_type = other RNA
agtttcaaactttctctatagagaataatattagccttctctgcgccacc

SEQ ID NO: 434 - RNA, 50 nt, Synthetic construct
  source 1..50: mol_type = other RNA
agtaccggtattacgcagttcgttttattatttcgacaatgtgcgccacc

SEQ ID NO: 435 - RNA, 50 nt, Synthetic construct
  source 1..50: mol_type = other RNA
aggactattttagcattaggtgatttggtaacaccgctttagacgccacc

SEQ ID NO: 436 - RNA, 50 nt, Synthetic construct
  source 1..50: mol_type = other RNA
aggagctgacccattagaatactgctcgtttaaactgattccctgccacc

SEQ ID NO: 437 - RNA, 50 nt, Synthetic construct
  source 1..50: mol_type = other RNA
aggctttgagacaataatttagttataagagaacccaaaaccaagccacc

SEQ ID NO: 438 - RNA, 50 nt, Synthetic construct
  source 1..50: mol_type = other RNA
aggaagatacctagctgagttgtctctatagaagagaaatgagagccacc

SEQ ID NO: 439 - RNA, 50 nt, Synthetic construct
  source 1..50: mol_type = other RNA
agcgagtaagcttgttctgtcatctaatatccatgcaaatggcagccacc

SEQ ID NO: 440 - RNA, 50 nt, Synthetic construct
  source 1..50: mol_type = other RNA
agtaaattagccaatcaaggtaactgtaaccttgtaccaagggagccacc

SEQ ID NO: 441 - RNA, 50 nt, Synthetic construct
  source 1..50: mol_type = other RNA
aggatacgagtgatacacattgtgaactacctatccagagttgcgccacc

SEQ ID NO: 442 - RNA, 50 nt, Synthetic construct
  source 1..50: mol_type = other RNA
agtgccactgtctgtgaaagtgatagactggccagctaatgagagccacc

SEQ ID NO: 443 - RNA, 50 nt, Synthetic construct
  source 1..50: mol_type = other RNA
aggatttcattgaacacattgcactgtttcaagttccgagcagcgccacc

SEQ ID NO: 444 - RNA, 50 nt, Synthetic construct
  source 1..50: mol_type = other RNA
agcacaatacgaacccagattctcttggtaccgaactgacaattgccacc

SEQ ID NO: 445 - RNA, 50 nt, Synthetic construct
  source 1..50: mol_type = other RNA
agtttggttcacttactcctggggagcatataatttatagaccagccacc

SEQ ID NO: 446 - RNA, 50 nt, Synthetic construct
  source 1..50: mol_type = other RNA
agctttagtcgcacccaacatattcgagtaggtaaaccatgccagccacc

SEQ ID NO: 447 - RNA, 50 nt, Synthetic construct
  source 1..50: mol_type = other RNA
agtatcatacttagacattgagctgaacgattaactgcagtccagccacc

SEQ ID NO: 448 - RNA, 50 nt, Synthetic construct
  source 1..50: mol_type = other RNA
agtactacacataaccgagtctgtagagtcataatatatctagagccacc

SEQ ID NO: 449 - RNA, 50 nt, Synthetic construct
  source 1..50: mol_type = other RNA
agatacggccacaactgaatcttttcgatagacctcagctagttgccacc

SEQ ID NO: 450 - RNA, 50 nt, Synthetic construct
  source 1..50: mol_type = other RNA
aggagatacaatacaggagatcgaaggacagtgcatctagcagtgccacc

SEQ ID NO: 451 - RNA, 50 nt, Synthetic construct
  source 1..50: mol_type = other RNA
agtaacacatgttgaacatttgttcgggtattagagtaagtgcggccacc

SEQ ID NO: 452 - RNA, 50 nt, Synthetic construct
  source 1..50: mol_type = other RNA
agttccacataaaggtagttgaacgtagtaatttaagatcccgagccacc

SEQ ID NO: 453 - RNA, 50 nt, Synthetic construct
  source 1..50: mol_type = other RNA
agtgtaaacattacccaactggttataattctctagcgcacggagccacc

SEQ ID NO: 454 - RNA, 50 nt, Synthetic construct
  source 1..50: mol_type = other RNA
agggtctaactaacaccaaggaagattaagtggtcagaatgcttgccacc

SEQ ID NO: 455 - RNA, 50 nt, Synthetic construct
  source 1..50: mol_type = other RNA
agagccgctcgtttcatcgctattgtagaaccacaatatttgcagccacc

SEQ ID NO: 456 - RNA, 92 nt, Synthetic construct
  source 1..92: mol_type = other RNA
agaacccgaacgcccttccgtcgctccaagggctagtggagcgttgctacccgccgacacttgctactaccagcaccaccgtcttcgccacc

SEQ ID NO: 457 - RNA, 92 nt, Synthetic construct
  source 1..92: mol_type = other RNA
agatcagacggtacatcgctacaagcgctagtacggagtgtaagccacaatttgaaacttgctactaccaggctgcgtagggattcgccacc

SEQ ID NO: 458 - RNA, 92 nt, Synthetic construct
  source 1..92: mol_type = other RNA
agtaggaacgccgctccgaggcctcattgtagcaggcgataccgtggcaccctacctcccaaccatccttcgccgccgcagtcttcgccacc

SEQ ID NO: 459 - RNA, 92 nt, Synthetic construct
  source 1..92: mol_type = other RNA
agcgtcgcgtcgttcagacgcaattacgtaatggcgtgggagaacgaacgtgccacccgcatatgcttctaccagtcggagtcttcgccacc

SEQ ID NO: 460 - RNA, 92 nt, Synthetic construct
  source 1..92: mol_type = other RNA
agtcggatggcctgatacaaagcgaccgtcgttccgacgcccaagttaccacgaatcaatgatgcggagaccgcgttacggacttcgccacc

SEQ ID NO: 461 - RNA, 92 nt, Synthetic construct
  source 1..92: mol_type = other RNA
agtatgacactccgaaggcctcaagtccaataaccgcacgtagccgacaaccttctaccacgcgtctaccaagtgtcccggacttcgccacc

SEQ ID NO: 462 - RNA, 92 nt, Synthetic construct
  source 1..92: mol_type = other RNA
agaaacgcgaataggagaatccgacgctacgcccttagctgcacattcggtctacctccccccactgagtctacgcttctaacttcgccacc

SEQ ID NO: 463 - RNA, 92 nt, Synthetic construct
  source 1..92: mol_type = other RNA
agggatagacgtccccgtcgctcgaagcgcgcgttgttgtggctgtcaggggtctaggatccagtttcggtgccgctgcagtcttcgccacc

SEQ ID NO: 464 - RNA, 92 nt, Synthetic construct
  source 1..92: mol_type = other RNA
aggcgttgaggacggcgctgcgtcgtgcataaaaccccacctagcttcgcgtgtgctctactgaactactacccgccgcagtcttcgccacc

SEQ ID NO: 465 - RNA, 92 nt, Synthetic construct
  source 1..92: mol_type = other RNA
agtgtaaacgtcactacgtcaacgctctcgacggtcgtctagcacagttgaccgtactaccccgcgtctaataattcccaatcttcgccacc

SEQ ID NO: 466 - RNA, 92 nt, Synthetic construct
  source 1..92: mol_type = other RNA
agatctttaaacgccggtgtacgcacggcttagggagtgccgtccaaattactgctctgtgtgccatcctgccgagtaaagtcttcgccacc

SEQ ID NO: 467 - RNA, 92 nt, Synthetic construct
  source 1..92: mol_type = other RNA
agtctgctgcggtattgtctctttgttaaaaggctagaactccagtcccgagtccgagtccttcctattctttgtgaccggtcttcgccacc

SEQ ID NO: 468 - RNA, 92 nt, Synthetic construct
  source 1..92: mol_type = other RNA
agcagcaccgacacccaccgttcagacgctcctgaatctgtattcaccccattggtcaatgacccctgaccgtgtgtaccagcttcgccacc

SEQ ID NO: 469 - RNA, 92 nt, Synthetic construct
  source 1..92: mol_type = other RNA
agcagggtgtccttcagacgctcctgaaaggaaggtggtctctcgcagttgtacctccctacctattgttctactactcatcctttgccacc

SEQ ID NO: 470 - RNA, 92 nt, Synthetic construct
  source 1..92: mol_type = other RNA
agactgagtctcgcatcgtcgcttctaaaaccagcagtggaggaatactacgatactacaattaccacgttccacccgcagtcttcgccacc

SEQ ID NO: 471 - RNA, 92 nt, Synthetic construct
  source 1..92: mol_type = other RNA
agcgcaaaaaaaatcgccaccgacggccagacgcaactataacgtggaccaggttcccccccgcgtctacgtacgccgcggtcttcgccacc

SEQ ID NO: 472 - RNA, 92 nt, Synthetic construct
  source 1..92: mol_type = other RNA
agcctgaattctaacgctcagtcgcacctggatcaaaatgcatccaggactaaactagttgtacattcgtaccagccgcagtcttcgccacc

SEQ ID NO: 473 - RNA, 92 nt, Synthetic construct
  source 1..92: mol_type = other RNA
agggttgggcaaagcaagatggccatcgcagcaacggcgatcgttactctactacgggtctaccaagcggcccaggcgcagtcttcgccacc

SEQ ID NO: 474 - RNA, 92 nt, Synthetic construct
  source 1..92: mol_type = other RNA
aggacattaagtgcaagtgtcgtacaacgctgaagaccctcaaagaacacgacactgcgcaactgtacgtactactaccagtcttcgccacc

SEQ ID NO: 475 - RNA, 92 nt, Synthetic construct
  source 1..92: mol_type = other RNA
agccgagtaagtgcaagcgccgcttcttcagcccggatggctatatcattcgccatacttgcgtttcgacgccgggcccagtcttcgccacc

SEQ ID NO: 476 - RNA, 92 nt, Synthetic construct
  source 1..92: mol_type = other RNA
agatcgcactgcgtgtaagcagcctctagccgcacgcgtcgttgcaatttttcgaccgtaccattgatattcgcgccgcagtcttcgccacc

SEQ ID NO: 477 - RNA, 92 nt, Synthetic construct
  source 1..92: mol_type = other RNA
agagggtaaccgtaacggtctatcgctgcatcgacaataggaccgctggttagtctacgtaccggaccactacctcctcagtcttcgccacc

SEQ ID NO: 478 - RNA, 92 nt, Synthetic construct
  source 1..92: mol_type = other RNA
agtgcatccgtcgctccaagtgcctgtgaccctcagaggtttgcaccataataacaggagtacctctaccagccgcccaagtcttcgccacc

SEQ ID NO: 479 - RNA, 92 nt, Synthetic construct
  source 1..92: mol_type = other RNA
aggacactttaccgtcacaaggtgtctttaactgattaactgcaagggagtcctctcctacgagtcaatgatcttccgtggacttcgccacc

SEQ ID NO: 480 - RNA, 92 nt, Synthetic construct
  source 1..92: mol_type = other RNA
agcgccgttgtagagaatactacgctcaaacgctagaaccgcaactaccagatcaccccctacctatgcttctacctcccgtcctcgccacc

SEQ ID NO: 481 - RNA, 92 nt, Synthetic construct
  source 1..92: mol_type = other RNA
aggtaggtgtagacacactaaattctttgggtggagtcttcccacacccgaagcgcaagaagcatagtgcatctcacactcacctagccacc

SEQ ID NO: 482 - RNA, 92 nt, Synthetic construct
  source 1..92: mol_type = other RNA
agactaaaaggacgccctctaaagggagaactctgcataaggcaggcgggcgagaccaaatcgcaggccttggctccttgaaccaagccacc

SEQ ID NO: 483 - RNA, 92 nt, Synthetic construct
  source 1..92: mol_type = other RNA
agtaaagacccagcagaaatctttgcatagaatgatgactccctcgatagaattttagcccactgcatccagccaataggagcccagccacc

SEQ ID NO: 484 - RNA, 92 nt, Synthetic construct
  source 1..92: mol_type = other RNA
aggactctgcttgagtgtgctctttgtatcagtgttggctggtctctgagaggaaggccttggctccttgaacttttgtctctcttgccacc

SEQ ID NO: 485 - RNA, 92 nt, Synthetic construct
  source 1..92: mol_type = other RNA
agaaggaccattaaaagtatgcccagtggagtctcctgaaataagtgctccttgaacttttggcaggccttggctccttgtacttggccacc

SEQ ID NO: 486 - RNA, 92 nt, Synthetic construct
  source 1..92: mol_type = other RNA
agaacgtgcacaatttgattcactccctcaagacggagcactctgcaggccttggctccttgaactgttggcccgcatgtcagcaggccacc

SEQ ID NO: 487 - RNA, 92 nt, Synthetic construct
  source 1..92: mol_type = other RNA
agatctagtgtataacttggataggaaaaaaattctttgggtggtgctattgctgacaccaactgaatccagccaataggagcccagccacc

SEQ ID NO: 488 - RNA, 92 nt, Synthetic construct
  source 1..92: mol_type = other RNA
agtcccactgcagtaaagggtaaacttcatgagatggggctgtttctggccttggctccttgaacttttaaattagccatctcatggccacc

SEQ ID NO: 489 - RNA, 92 nt, Synthetic construct
  source 1..92: mol_type = other RNA
agtgtactcctctttgcctggactaagcactgcctctcaaaggagtaagccttggctccttgaacttttttgctccatcgcagtaggccacc

SEQ ID NO: 490 - RNA, 92 nt, Synthetic construct
  source 1..92: mol_type = other RNA
aggaaaaaaaaatctctctcgggtggagtcttcttcaatgcttgcctaaaaagactaggctcttggcgcatcgcgccacacacctagccacc

SEQ ID NO: 491 - RNA, 92 nt, Synthetic construct
  source 1..92: mol_type = other RNA
agcaaaaaaaaaaaaacccctcgggtggagtcctcgcaaagattacgttattccagatccttaagctgacagccaataggagcccagccacc

SEQ ID NO: 492 - RNA, 92 nt, Synthetic construct
  source 1..92: mol_type = other RNA
aggatatcttatttaagtactcctcgggtggagtcctcacaggcctaggctccttgaggcaattgccttccgtcaataggagcccagccacc

SEQ ID NO: 493 - RNA, 92 nt, Synthetic construct
  source 1..92: mol_type = other RNA
agaccctgagtagaagagttaaataaaacactagtccactcgaggtctaaccagcaggccttggctccttgaacttatatcacctagccacc

SEQ ID NO: 494 - RNA, 92 nt, Synthetic construct
  source 1..92: mol_type = other RNA
agatactactccctgaatactctgtggggagtcctctcatacaggcaggcctaggctccttgaactttaggccgccatcgaatgaggccacc

SEQ ID NO: 495 - RNA, 92 nt, Synthetic construct
  source 1..92: mol_type = other RNA
agacacctcgggtggagtcctgagaaggtatgaattataaaacgtagcagtacatcaaacatacggccttggctccttgaacttttgccacc

SEQ ID NO: 496 - RNA, 92 nt, Synthetic construct
  source 1..92: mol_type = other RNA
aggaccccgaatacctctcgggtggagtctcctcccagacaaggcttaccgtaagaatattagattcctgaggcaataggagcccagccacc

SEQ ID NO: 497 - RNA, 92 nt, Synthetic construct
  source 1..92: mol_type = other RNA
agcctgccccaaactaactcctcgggtggagtctcctgaatgaggacgcgctgtttaatgaaacaaaacgaaccaataggagcccagccacc

SEQ ID NO: 498 - RNA, 92 nt, Synthetic construct
  source 1..92: mol_type = other RNA
aggtctctcaacatacctctcgggtggagtcttctgaatggatagagttttgctggagatgaagccattggctgaaagcacacctagccacc

SEQ ID NO: 499 - RNA, 92 nt, Synthetic construct
  source 1..92: mol_type = other RNA
agctctcgggtggagtcttctaaaaatttaacaaggctaatcctatggtggttgatgtcgaaatcacatagcgcaataggagcccagccacc

SEQ ID NO: 500 - RNA, 92 nt, Synthetic construct
  source 1..92: mol_type = other RNA
agtctatactcgaaggaaaagctctcgggtggagtcttctgcctataatagcacaatcaaaaggcatcataagtaataggagcccagccacc

SEQ ID NO: 501 - RNA, 92 nt, Synthetic construct
  source 1..92: mol_type = other RNA
agatgaggaagcttgtccgaaggcatagtccagtcctcccttctttaggccttggctccttgaacttttggaagtaattagagtaggccacc

SEQ ID NO: 502 - RNA, 92 nt, Synthetic construct
  source 1..92: mol_type = other RNA
agaacggaaggactctgtggagaaacaaaactcttcgggtggagtctacagttggatgaagaggaatagaaggcaataggagcccagccacc

SEQ ID NO: 503 - RNA, 92 nt, Synthetic construct
  source 1..92: mol_type = other RNA
agtggcgctaaactaactcctcgggtggagtctcgcctctctatacatggtaagcattagaaccatatctctttcacactcacctagccacc

SEQ ID NO: 504 - RNA, 92 nt, Synthetic construct
  source 1..92: mol_type = other RNA
agatctcgggtggagtcctgaggatgaaggtgatgaacatcctacctagaggcgtaatatcactgcatccagccaataggagcccagccacc

SEQ ID NO: 505 - RNA, 92 nt, Synthetic construct
  source 1..92: mol_type = other RNA
agtcctccgggtggagtcctctcaaagaagaagaaactaaagaaaaaggccttggctccttgaacttttggccgccatgtagagaggccacc

SEQ ID NO: 506 - RNA, 92 nt, Synthetic construct
  source 1..92: mol_type = other RNA
aggagcatggtgacgataatcttgagggtgagctgagattcagaggataacagaaggccttggctccttgaacttttgcccgttatgccacc

SEQ ID NO: 507 - RNA, 92 nt, Synthetic construct
  source 1..92: mol_type = other RNA
agaactgctaaactgccccagaaaattgtcatgaaaaatactaatgagaaggccttggctccttgaacttttggccgccatgtgtcgccacc

SEQ ID NO: 508 - RNA, 92 nt, Synthetic construct
  source 1..92: mol_type = other RNA
agatctctctcgggtggaatcctgggaaggtatttattaacttaacgatggccttggctccttgaacttttggccgtccttcagaggccacc

SEQ ID NO: 509 - RNA, 92 nt, Synthetic construct
  source 1..92: mol_type = other RNA
agaataattcttcgcgtgcagtattcagacggagtagtcttcccacacctcaggccttggctccttgaacttttggccgccatgtcgccacc

SEQ ID NO: 510 - RNA, 92 nt, Synthetic construct
  source 1..92: mol_type = other RNA
agtcaaagacctcgggtggagtcttcaggcgaagatctctctgtttgcgtcaggccttggctccttgaacttttggccaccatgtggccacc

SEQ ID NO: 511 - RNA, 92 nt, Synthetic construct
  source 1..92: mol_type = other RNA
agctctggggtggattcttctagatgaaacacactaaattaaactattaacgaaagccttggctccttgaacttttggccgccatggccacc

SEQ ID NO: 512 - RNA, 92 nt, Synthetic construct
  source 1..92: mol_type = other RNA
agaaactttctcgggtggagtctttagatatccatcctagccagacgactgaaggccttggctccttgaacttttggcaacttgtggccacc

SEQ ID NO: 513 - RNA, 92 nt, Synthetic construct
  source 1..92: mol_type = other RNA
agccctggggtggagtcctctgcactcacatcatttcaacctaaagcagatcacgatatctcaggccttggctccttgaacttttggccacc

SEQ ID NO: 514 - RNA, 92 nt, Synthetic construct
  source 1..92: mol_type = other RNA
agctctagggtagcgtcctctgaacaaactacagacctggcttagatcatagaaggccttggctccttgaacttttggacgccatggccacc

SEQ ID NO: 515 - RNA, 92 nt, Synthetic construct
  source 1..92: mol_type = other RNA
aggtaaacaaaaaaaaaagaactctctcgggtggagtcttcggacgtacggaaggccttggctccttgaacttttggccgccattggccacc

SEQ ID NO: 516 - RNA, 92 nt, Synthetic construct
  source 1..92: mol_type = other RNA
agacactaagttctcgcccggagaagtctcatcaaataattctcgctgcaggccttggctccttgaacttttggccgccaagtgtcgccacc

SEQ ID NO: 517 - RNA, 92 nt, Synthetic construct
  source 1..92: mol_type = other RNA
agccctgtctgaaaggcctcgggtggagtcctctcacgcccacgctagtcgacatctcaggccttggctccttgaacttttggccggccacc

SEQ ID NO: 518 - RNA, 92 nt, Synthetic construct
  source 1..92: mol_type = other RNA
agtcctctgagacaggactcgggtggaggcttacttgttaattaggcaccgtcatctcaggccttggctccttgaacttttggactgccacc

SEQ ID NO: 519 - RNA, 92 nt, Synthetic construct
  source 1..92: mol_type = other RNA
agattgtctctcgggtgcaatctacggactaaaaagacctctcagttacggaaggccttggctccttgaacttttggccgccattggccacc

SEQ ID NO: 520 - RNA, 92 nt, Synthetic construct
  source 1..92: mol_type = other RNA
agtgtatggatctctcagaagtaacccttcgggtggagtcttcccacttgaacttttggccgccaccgcaggccttggctccttgagccacc

SEQ ID NO: 521 - RNA, 92 nt, Synthetic construct
  source 1..92: mol_type = other RNA
agaactctcgtgaggtcctctgaactcacttgaattcttgtcctgcgcattcgcgataccttaggccttggctccttgaacttttggccacc

SEQ ID NO: 522 - RNA, 92 nt, Synthetic construct
  source 1..92: mol_type = other RNA
aggactaaagtactccccaggcgaattcacgtcaaaagacctctcaggaaggccttggctccttgaacttttggccgccatgtgtcgccacc

SEQ ID NO: 523 - RNA, 92 nt, Synthetic construct
  source 1..92: mol_type = other RNA
agccaaaagaacttaaatctctctcgggtggagtcttatgaaaggattacagaatacatctcaggccttggctcctcgaccttttggccacc

SEQ ID NO: 524 - RNA, 92 nt, Synthetic construct
  source 1..92: mol_type = other RNA
agtaataaatctctctcgggtggagtctcatgcacacgtgctaggcgagtttttgatcccgaggctctgtgagtaccacacacctagccacc

SEQ ID NO: 525 - RNA, 92 nt, Synthetic construct
  source 1..92: mol_type = other RNA
agtcctcctgaatgaccggagaagtcctctcaaaaattgacataggggaaggccttggctccttgaacttttggccgcaacgtgaggccacc

SEQ ID NO: 526 - RNA, 92 nt, Synthetic construct
  source 1..92: mol_type = other RNA
agcacagaaaaagtacttctctgaagtctcataacaaaaccagacaggaaggccttggctccttgaacttttggccaacatttcccgccacc

SEQ ID NO: 527 - RNA, 92 nt, Synthetic construct
  source 1..92: mol_type = other RNA
agactacctgtgaactcggagaaggagttcccccaggcttgcatacagtaggccttggctccttgaacttttggccgccatgacgtgccacc

SEQ ID NO: 528 - RNA, 92 nt, Synthetic construct
  source 1..92: mol_type = other RNA
agaccgacagaggactcgcagaaggacttcctattgaatctattgtggaaggccttggctccttgaacttttgaccgccatgtgaggccacc

SEQ ID NO: 529 - RNA, 92 nt, Synthetic construct
  source 1..92: mol_type = other RNA
agaatctctctcgggtggagtcttcccacagttcacacataaaatttatcgcgaggctctgtgagtaccacacagtgggattactagccacc

SEQ ID NO: 530 - RNA, 92 nt, Synthetic construct
  source 1..92: mol_type = other RNA
aggctctcgtgtggagtcttgtcaaataaaactttgggtggtggacggccttggctccttgaacttttggccgccatgtggagcaggccacc

SEQ ID NO: 531 - RNA, 92 nt, Synthetic construct
  source 1..92: mol_type = other RNA
agatctctcacgggttgaatcatgtgaaagtaaactaaatggatcggcaggccttggctccttgaacttttggccgaccaccataggccacc

SEQ ID NO: 532 - RNA, 92 nt, Synthetic construct
  source 1..92: mol_type = other RNA
agacttaaatctctctcgggtggagtcttctggtaaaagaaaaaattctccaagagcaaacagcaggccttggctccttgaacaaagccacc

SEQ ID NO: 533 - RNA, 76 nt, Synthetic construct
  source 1..76: mol_type = other RNA
agataatgcaaaaatcaaaatcaatcatcatcacaacatcaacaatcaatcatcaacacatcatcaagacgccacc

SEQ ID NO: 534 - RNA, 69 nt, Synthetic construct
  source 1..69: mol_type = other RNA
agataatcactcctccccatcctctccctctgtccctctgtccctctgaccctgcactgtcccgccacc

SEQ ID NO: 535 - RNA, 80 nt, Synthetic construct
  source 1..80: mol_type = other RNA
agataatacagcagttacactgcggcgggcgtctgttctagtgtttgagccgtcgtgcttcaccggtctacctcgccacc

SEQ ID NO: 536 - RNA, 166 nt, Synthetic construct
  source 1..166: mol_type = other RNA
agataatctttctgcagcaggaaccgcggctgctggacaagaggggtgcggtggatactgacctttgctccggcctcgtgtagcgtgaagacacagcgcatctccccgctgtaggcttcctcccacagaacccgtttcgggcctcagagcgtctggtgaggccacc

SEQ ID NO: 537 - RNA, 102 nt, Synthetic construct
  source 1..102: mol_type = other RNA
agataattctacagcattgtcccagatatagccgttggtacaaagcggggatctgacgagcttttctctacttgtgtcactaacggaccgtttatcgccacc

SEQ ID NO: 538 - RNA, 190 nt, Synthetic construct
  source 1..190: mol_type = other RNA
agataatcgccgccccttccgaggctaaatcggctgcgttcctctcggaacgcgccgcagaaggggtcctggtgacgagtcccgcgttctctccttgaatccactcgccagcccgccgccctctgccgccgcaccctgcacacccgcccctctcctgtgccaggaacttgctactaccagcaccgccacc

SEQ ID NO: 539 - RNA, 130 nt, Synthetic construct
  source 1..130: mol_type = other RNA
agataatcacatatgtatccacagcgcctagaacactgcctgtaacgtggaaggtgttcgatctatagagttttgtcgaatgaatgaatgaagccgactagtgcacagggagtgcagcggcgcggccacc

SEQ ID NO: 540 - RNA, 68 nt, Synthetic construct
  source 1..68: mol_type = other RNA
agataatacttccccgtcaccagctcctgtgcctgccagtcggtgcccctcccgctccagccgccacc

SEQ ID NO: 541 - RNA, 85 nt, Synthetic construct
  source 1..85: mol_type = other RNA
agataatctcagtcgccgctgccagctctcgcactctgttcttccgccgctccgccgtcgcgtttctctgccggtcgcagccacc

SEQ ID NO: 542 - RNA, 91 nt, Synthetic construct
  source 1..91: mol_type = other RNA
agataatctcattgaactcgcctgcagctcttgggttttttgtggcttccttcgttattggagccaggcctacaccccagcaaccgccacc

SEQ ID NO: 543 - RNA, 129 nt, Synthetic construct
  source 1..129: mol_type = other RNA
agataatagtatctgtgggtacccggagcacggagatctcgccggctttacgttcacctcggtgtctgcagcaccctccgcttcctctcctaggcgacgagacccagtggctagaagttcaccgccacc

SEQ ID NO: 544 - RNA, 102 nt, Synthetic construct
  source 1..102: mol_type = other RNA
agataatccagttactctagcgcgccaggccgaaccgcagcttcttggcttaggtacttctactcacagcggccgattccgaggccaactccagcagccacc

SEQ ID NO: 545 - RNA, 225 nt, Synthetic construct
  source 1..225: mol_type = other RNA
agataatagtgaattcaccaccccagctctctgcggattgagctagtgcatcttttggaaaaatacctattatttgtgaggtcttcaaaagttgtgacagccctgattcagatcaggtagctttcacatttgtccattgctattttgagaaaaagcatttaaaaacaatatgtggtcttatttttggtatacgggattttaaaaaaaaggtgctgccacgccacc

SEQ ID NO: 546 - RNA, 207 nt, Synthetic construct
  source 1..207: mol_type = other RNA
agataatgcgagtaagtgcaggtgccggtggcgcacggggctcgggtagttctgggaacctctgggcggtcctgggactgaggagtcggcgccttcctcaagaatgcctgggacaaggagccagtgctggtcgtgtccttcgtcgtcgggggcctcgctgtaattctgcccccattgagcccctacttcaagtactccgtcgccacc

SEQ ID NO: 547 - RNA, 266 nt, Synthetic construct
  source 1..266: mol_type = other RNA
agataataaaggttcctgcctctcctcggccaggcggaacctctctgctgggcccggtggccgcaaaagaactttctttctcccgcccgaacggtcgccgcggccaactgcctcgcccgcctggcagcctaaccctccttctcttcttctcctctccggcttcgcgcggccctgcctccctctcgcccggcggcatccgcttgctgctgccaccgcctcctcatcttctgcccggccaaccggcctgccccgctgcagtggccacc

SEQ ID NO: 548 - RNA, 79 nt, Synthetic construct
  source 1..79: mol_type = other RNA
agataatagaacagacagacctactgacacaggggaggctctgtgagtaccacacagtggggagggggtgggggccacc

SEQ ID NO: 549 - RNA, 81 nt, Synthetic construct
  source 1..81: mol_type = other RNA
agataatcataaaaccagctgagtatttgtgccaggaagactgcgtgcagaaggccttggctccttgaacttttggccacc

SEQ ID NO: 550 - RNA, 64 nt, Synthetic construct
  source 1..64: mol_type = other RNA
agataatagcaaggctcagcctcaagattcacagcatctcagacacagcctaggccgcgccacc

SEQ ID NO: 551 - RNA, 216 nt, Synthetic construct
  source 1..216: mol_type = other RNA
agataataatctagttctctgtgttcgggcggggggagtcgaactcgtggggtgtcagtatcccgggttcgtctccccactaccggttcccaccactgagtctgggggcgaaggaaggagccagagtgcaattgcagatccagaccccagagtcagaaggagtgagaaccctgacccctaatcccactgcatccagccaataggagcccagccacc

SEQ ID NO: 552 - RNA, 397 nt, Synthetic construct
  source 1..397: mol_type = other RNA
agataataatcagggctcaactatggctgcctcctcctcttcctctggatcaggaggcgcagggagcagggagatgtagacctcattgacctctgtgtccaaatgtcggccaccccgaggtgcttccaggtttaagatgccatcatgggagagagcagctcggactcgccaggggtcgacatcagcaggcaggacatagctcagtgagtactgggtatgtgtcacattgccaaatcccggatcacaagtctccatgaactgctggtgagctaggataataaaacccctgacatcaccattccagaagcttcacaagactgcatatataaggggctggctgtagctgcagctgaaggagctgaccagccagctgacccctcacactcacctagccacc

SEQ ID NO: 553 - RNA, 204 nt, Synthetic construct
  source 1..204: mol_type = other RNA
agataatgtttccagggggagaactctgcagaggagaggcaaagttcaacaggacaacaaagaagcagcagaaacagccaggaaaacgaagagcccccaggttttgacacagaaccctccaccagagttctcaaatccccaaatactctgcaaagtgcaacttgacgggaacttttcaactgacttttacttttgacggccacc

SEQ ID NO: 554 - RNA, 103 nt, Synthetic construct
  source 1..103: mol_type = other RNA
agataattcctcctcgcctgccaccggtgcacccagtccgctcacccagcccagtccgtccggtcctcaccgcctgccggccggcccaccccccaccgccacc

SEQ ID NO: 555 - RNA, 215 nt, Synthetic construct
  source 1..215: mol_type = other RNA
agataatggtgagctgcatgatgtcttacatttcatgagcttcctctaaatctagttgtatttttcgtctgaggggggctgaggctgaggtggcctccttgaaccgtaggatccagctggttgaagaagagctggaccgtgctcaggagcgcctggccactgccctgcaaaagctggaagaagctgaaaaagctgctgatgagagtgaggccacc

SEQ ID NO: 556 - RNA, 82 nt, Synthetic construct
  source 1..82: mol_type = other RNA
agataatgcctgcagccggtgctgtccacagggagctccagcccttctcacactcgacccgcagaaaccacccaccgccacc

SEQ ID NO: 557 - RNA, 84 nt, Synthetic construct
  source 1..84: mol_type = other RNA
agataatggcactcgtggctgtggtggcttcggcagcggcttcagcagatcggcggcatcagcggtagcaccagcactgccacc

SEQ ID NO: 558 - RNA, 59 nt, Synthetic construct
  source 1..59: mol_type = other RNA
agataatcctgctttctgcattcttctctccacatccctctctgtacttacaggccacc

SEQ ID NO: 559 - RNA, 139 nt, Synthetic construct
  source 1..139: mol_type = other RNA
agataatagctgcgcctggctgcgcacagagctccctcccaggcccgcgaacttggccattcagccgccgctgtccccgctgcgcgccctcgcgcctctgcctgagaagccaggcgctgttcccccaccccaggccacc

SEQ ID NO: 560 - RNA, 82 nt, Synthetic construct
  source 1..82: mol_type = other RNA
agataataactcgccgctgccggcctgacctcgctcccagccctgctgcccagattctaggctccaagctcaggacgccacc

SEQ ID NO: 561 - RNA, 378 nt, Synthetic construct
  source 1..378: mol_type = other RNA
agataatacacggtaagagggagagcccaggtttgaacaagtctatctgattccaaagcctaaggtccttcttagcactcttcagcctgtttagataactggggaaggggagggtgggtgctctcccagaagcatattcattcagtaaactcagagcatctattttgtgccactctgcacggtactgaacgtacaaagatgatgaagtggtctctgtgggccttgggagcccatgttcaaggcaggaccattggaaaacagcatccctcatgcttgacccggatggggcaggacattgatgaatcgtgatggagggggtgactatgggatgcttcaaaaaaaaggcttcctggaggagatgacatttaactggccacc

SEQ ID NO: 562 - RNA, 71 nt, Synthetic construct
  source 1..71: mol_type = other RNA
agataatatctttgggtggtggagtgcaaaggaggcgacctgcaacagaggagtcccggtcaccagccacc

SEQ ID NO: 563 - RNA, 45 nt, Synthetic construct
  source 1..45: mol_type = other RNA
agataattggcgggcattcctgaagctgacagcattcgggccacc

SEQ ID NO: 564 - RNA, 86 nt, Synthetic construct
  source 1..86: mol_type = other RNA
agataatgcaggcggagtgcctgagtgccctctcactcaaccctctctaccctttgtctgcagaggcggccgctgacagcgccacc

SEQ ID NO: 565 - RNA, 53 nt, Synthetic construct
  source 1..53: mol_type = other RNA
aggaaaaacgcccctgacacaagtgtgttcactaccaacctcccacagccacc

SEQ ID NO: 566 - RNA, 255 nt, Synthetic construct
  source 1..255: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaacccca

SEQ ID NO: 567 - RNA, 133 nt, Synthetic construct
  source 1..133: mol_type = other RNA
gctcgctttcttgctgtccaatttctattaaaggttcctttgttccctaagtccaactactaaactgggggatattatgaagggccttgagcatctggattctgcctaataaaaaacatttattttcattgca

SEQ ID NO: 568 - RNA, 120 nt, Synthetic construct
  source 1..120: mol_type = other RNA
gtgtgtggaggacaccctgaaccccccgctttcaaacaagttttcaaattgtttgaggtcaggatttctcaaactgattcctttctttgcatatgagtatttgaaaataaatattttccc

SEQ ID NO: 569 - RNA, 83 nt, Synthetic construct
  source 1..83: mol_type = other RNA
ttgaggatagtcaggagcgtcaatgtgcttgtacatagagtgctgtagctgtgtgttccaataaattattttgtagggaaagt

SEQ ID NO: 570 - RNA, 130 nt, Synthetic construct
  source 1..130: mol_type = other RNA
cctcgccccggacctgccctcccgccaggtgcacccacctgcaataaatgcagcgaagccgggacctcctcgcgggccaccacgcctcccgcgtccaccaccgtggaccaataaatcgtgcgaagggccc

SEQ ID NO: 571 - RNA, 77 nt, Synthetic construct
  source 1..77: mol_type = other RNA
acatctcttgccatcacctagctgcctgcacctgcccttcagggagatgggggtcattaaaggaaactgaacattga

SEQ ID NO: 572 - RNA, 113 nt, Synthetic construct
  source 1..113: mol_type = other RNA
taagctggagcctcggtggccatgcttcttgccccttgggcctccccccagcccctcctccccttcctgcacccgtacccccgtggtctttgaataaagtctgagtgggcggc

SEQ ID NO: 573 - RNA, 110 nt, Synthetic construct
  source 1..110: mol_type = other RNA
gctggagcctcggtggcctagcttcttgccccttgggcctccccccagcccctcctccccttcctgcacccgtacccccgtggtctttgaataaagtctgagtgggcggc

SEQ ID NO: 574 - RNA, 278 nt, Synthetic construct
  source 1..278: mol_type = other RNA
accgaatatctccaatgctctcccctttcatatctagcgtggtccggaatggaaaagtgggactggatgctacaaactgtttgaggataactgacttaaaatctggctgcacatcattgactcctgggcccaactgtgaccgatttaaactgcacataccatatgctggagagacattaaagtgggatatcattttcaatgcccaatacccagaactgcctcccgattttatctttggagaagatgctgaattcctgccagacccctcagctttgcag

SEQ ID NO: 575 - RNA, 223 nt, Synthetic construct
  source 1..223: mol_type = other RNA
ggatagaggggaggccttattgtatcggttttatattacctgttctgcttcaccaggagatcatgctgctgtgatactgagttttctaaacagcataaggaagacttgctcccctgtcctatgaaagagaatagttttggaggggagaagtgggacaaaaaagatgcagttttcctttgtattgggaaatgtgaaaataaaattgtcaactctttcagttaaa

SEQ ID NO: 576 - RNA, 288 nt, Synthetic construct
  source 1..288: mol_type = other RNA
acacactccacctccagcacgcgacttctcaggacgacgaatcttctcaatgggggggcggctgagctccagccaccccgcagtcactttctttgtaacaacttccgttgctgccatcgtaaactgacacagtgtttataacgtgtacatacattaacttattacctcattttgttatttttcgaaacaaagccctgtggaagaaaatggaaaacttgaagaagcattaaagtcattctgttaagctgcgtaaatggtctttgaataaagcctgagtaggaagtctag

SEQ ID NO: 577 - RNA, 378 nt, Synthetic construct
  source 1..378: mol_type = other RNA
catcacatttaaaagcatctcagcctaccatgagaataagagaaagaaaatgaagatcaaaagtctattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaatggtctttgaataaagcctgagtaggaagtctag

SEQ ID NO: 578 - RNA, 142 nt, Synthetic construct
  source 1..142: mol_type = other RNA
tgataatagtccataaagtaggaaacactacagctggagcctcggtggccatgcttcttgccccttgggcctccccccagcccctcctccccttcctgcacccgtacccccgtggtctttgaataaagtctgagtgggcggc

SEQ ID NO: 579 - RNA, 142 nt, Synthetic construct
  source 1..142: mol_type = other RNA
tgataataggctggagcctcggtggctccataaagtaggaaacactacacatgcttcttgccccttgggcctccccccagcccctcctccccttcctgcacccgtacccccgtggtctttgaataaagtctgagtgggcggc

SEQ ID NO: 580 - RNA, 141 nt, Synthetic construct
  source 1..141: mol_type = other RNA
tgataataggctggagcctcggtggccatgcttcttgccccttgggcctccccccagtccataaagtaggaaacactacacccctcctccccttcctgcacccgtacccccgtggtctttgaaaaagtctgagtgggcggc

SEQ ID NO: 581 - RNA, 142 nt, Synthetic construct
  source 1..142: mol_type = other RNA
tgataataggctggagcctcggtggccatgcttcttgccccttgggcctccccccagcccctcctccccttctccataaagtaggaaacactacactgcacccgtacccccgtggtctttgaataaagtctgagtgggcggc

SEQ ID NO: 582 - RNA, 142 nt, Synthetic construct
  source 1..142: mol_type = other RNA
tgataataggctggagcctcggtggccatgcttcttgccccttgggcctccccccagcccctcctccccttcctgcacccgtaccccctccataaagtaggaaacactacagtggtctttgaataaagtctgagtgggcggc

SEQ ID NO: 583 - RNA, 142 nt, Synthetic construct
  source 1..142: mol_type = other RNA
tgataataggctggagcctcggtggccatgcttcttgccccttgggcctccccccagcccctcctccccttcctgcacccgtacccccgtggtctttgaataaagttccataaagtaggaaacactacactgagtgggcggc

SEQ ID NO: 584 - RNA, 164 nt, Synthetic construct
  source 1..164: mol_type = other RNA
tgataatagtccataaagtaggaaacactacagctggagcctcggtggccatgcttcttgccccttgggcctccccccagcccctcctccccttcctgcacccgtaccccccgcattattactcacggtacgagtggtctttgaataaagtctgagtgggcggc

SEQ ID NO: 585 - RNA, 164 nt, Synthetic construct
  source 1..164: mol_type = other RNA
tgataatagtccataaagtaggaaacactacagctggagcctcggtggcctagcttcttgccccttgggcctccccccagcccctcctccccttcctgcacccgtaccccccgcattattactcacggtacgagtggtctttgaataaagtctgagtgggcggc

SEQ ID NO: 586 - RNA, 141 nt, Synthetic construct
  source 1..141: mol_type = other RNA
tgataaaggctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 587 - RNA, 142 nt, Synthetic construct
  source 1..142: mol_type = other RNA
tgataataggctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 588 - RNA, 118 nt, Synthetic construct
  source 1..118: mol_type = other RNA
tgataataggctggagcctcggtggccatgcttcttgccccttgggcctcccccagcccctcctccccttcctgcacccgtacccccgtggtctttgaataaagtctgagtgggcggc

SEQ ID NO: 589 - RNA, 163 nt, Synthetic construct
  source 1..163: mol_type = other RNA
tgataataggctggagcctcggtggccatgcttcttgccccttgggcccaaacaccattgtcacactccatccccccagcccctcctccccttcctccataaagtaggaaacactacatgcacccgtacccccgtggtcttgaataaagtctgagtgggcggc

SEQ ID NO: 590 - RNA, 370 nt, Synthetic construct
  source 1..370: mol_type = other RNA
gcgcctgcccacctgccaccgactgctggaacccagccagtgggagggcctggcccaccagagtcctgctccctcactcctcgccccgccccctgtcccagagtcccacctgggggctctctccacccttctcagagttccagtttcaaccagagttccaaccaatgggctccatcctctggattctggccaatgaaatatctccctggcagggtcctcttcttttcccagagctccaccccaaccaggagctctagttaatggagagctcccagcacactcggagcttgtgctttgtctccacgcaaagcgataaataaaagcattggtggcctttggtctttgaataaagcctgagtaggaagtctag

SEQ ID NO: 591 - RNA, 566 nt, Synthetic construct
  source 1..566: mol_type = other RNA
gcccctgccgctcccacccccacccatctgggccccgggttcaagagagagcggggtctgatctcgtgtagccatatagagtttgcttctgagtgtctgctttgtttagtagaggtgggcaggaggagctgaggggctggggctggggtgttgaagttggctttgcatgcccagcgatgcgcctccctgtgggatgtcatcaccctgggaaccgggagtggcccttggctcactgtgttctgcatggtttggatctgaattaattgtcctttcttctaaatcccaaccgaacttcttccaacctccaaactggctgtaaccccaaatccaagccattaactacacctgacagtagcaattgtctgattaatcactggccccttgaagacagcagaatgtccctttgcaatgaggaggagatctgggctgggcgggccagctggggaagcatttgactatctggaacttgtgtgtgcctcctcaggtatggcagtgactcacctggttttaaaaaacaacctgcaacatctcatggtctttgaataaagcctgagtaggaagtctag

SEQ ID NO: 592 - RNA, 378 nt, Synthetic construct
  source 1..378: mol_type = other RNA
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaatggtctttgaataaagcctgagtaggaagtctag

SEQ ID NO: 593 - RNA, 117 nt, Synthetic construct
  source 1..117: mol_type = other RNA
gctgccttctgcggggcttgccttctggccatgcccttcttctctcccttgcacctgtacctcttggtctttgaataaagcctgagtaggaaggcggccgctcgagcatgcatctag

SEQ ID NO: 594 - RNA, 116 nt, Synthetic construct
  source 1..116: mol_type = other RNA
taataggctggagcctcggtggccatgcttcttgccccttgggcctccccccagcccctcctccccttcctgcacccgtacccccgtggtctttgaataaagtctgagtgggcggc

SEQ ID NO: 595 - RNA, 118 nt, Synthetic construct
  source 1..118: mol_type = other RNA
tgataataggctggagcctcggtggccatgcttcttgccccttgggcctccccccagcccctcctccccttcctgcacccgtaccccctggtctttgaataaagtctgagtgggcggc

SEQ ID NO: 596 - RNA, 119 nt, Synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 597 - RNA, 119 nt, Synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 598 - RNA, 96 nt, Synthetic construct
  source 1..96: mol_type = other RNA
agcgctgccttctgcggggcttgccttctggccatgcccttcttctctcccttgcacctgtacctcttggtctttgaataaagcctgagtaggaag

SEQ ID NO: 599 - RNA, 102 nt, Synthetic construct
  source 1..102: mol_type = other RNA
tgataataggctgccttctgcggggcttgccttctggccatgcccttcttctctcccttgcacctgtacctcttggtctttgaataaagcctgagtaggaag

SEQ ID NO: 600 - RNA, 169 nt, Synthetic construct
  source 1..169: mol_type = other RNA
ggccaggccccttccctctgcccccgggtgcttgaagtctagccccatcctggtccaatgcgctcttggtagcctcctttcccagctgcccgcccgccgccatgccgcccttactgcccctgcgcctgtgccggctgtggccccgcaaccctccctcccggctcctcgg

SEQ ID NO: 601 - RNA, 143 nt, Synthetic construct
  source 1..143: mol_type = other RNA
tgcccgtcctcaccaagactgactgcctgctgctttgctactgcccgggcccatgagactgacttcccactgctctgcctgcctctccccactgcactggcacagccccgccttgccgctgctgatccattgccggtgtgacc

SEQ ID NO: 602 - RNA, 141 nt, Synthetic construct
  source 1..141: mol_type = other RNA
tgctgcctgcttcttgctccagcaccatggaatgcctgcgcagtttaccctgcctcctgccccgcgcgatgagacttccccggcggacgctgtgtgccctggccttggacgtgacctctgtgggtcctcccgttgctgcct

SEQ ID NO: 603 - RNA, 149 nt, Synthetic construct
  source 1..149: mol_type = other RNA
ttccagccagacacccgccccccggccctggctaagaagttgcttcctgttgccagcatgacctaccctcgcctctttgatgccatccgctgccacctccttttgctcctggaccctttagcctctctgcccttccactctctgacccc

SEQ ID NO: 604 - RNA, 155 nt, Synthetic construct
  source 1..155: mol_type = other RNA
gccttggctcctccaggaaggctcaggagccctacctccctgccattatagctgctccccgccagaagcctgtgccaactctctgcattccctgatctccatccctgtggctgtcacccttggtcacctccgtgctgtcactgccatctcccccc

SEQ ID NO: 605 - RNA, 190 nt, Synthetic construct
  source 1..190: mol_type = other RNA
ctgacagcgtgggcaacgcctgccgcctgctctgaggcccgatccagtgggcaggccaaggcctgctgggcccccgcggacccaggtgctctgggtcacggtccctgtccccgcacccccgcttctgtctgccccattgtggctcctcaggctctctcccctgctctcccacctctacctccacccccac

SEQ ID NO: 606 - RNA, 233 nt, Synthetic construct
  source 1..233: mol_type = other RNA
ctttgcaggatgaaacacttccccgcttggctctcattcttccacaagagagacctttctccggacctggttgctactggttcagcaactctgcagaaaatgtcctcccctgtggctgcctcagctcatgcctttggcctgaagtcccagcattgatggcagcccctcatcttccaagttttgtgctcccctttacctaacgcttcctgcctcccatgcatctgtactcctcc

SEQ ID NO: 607 - RNA, 169 nt, Synthetic construct
  source 1..169: mol_type = other RNA
ccttgagcttggagtcctcctcctccagtaggcgctgcgtctccttcttacgttccagctggtcgaggcgccgcttctccttctcctccttgcgctgctccttcctcatgacgtgtttgtcgtcgtccttccagtaggcatcctccagctccttctgcttcttggcatc

SEQ ID NO: 608 - RNA, 276 nt, Synthetic construct
  source 1..276: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaaccctcgagctcgctttcttgctgtccaatttctattaaaggttcctttgttccctaagtccaactactaaactgggggatattatgaagggccttgagcatctggattctgcctaataaaaaacatttattttcattgcgaa

SEQ ID NO: 609 - RNA, 597 nt, Synthetic construct
  source 1..597: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcccctcgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 610 - RNA, 286 nt, Synthetic construct
  source 1..286: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaaccctcgaaggcaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 611 - RNA, 277 nt, Synthetic construct
  source 1..277: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaaccctcgactggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 612 - RNA, 288 nt, Synthetic construct
  source 1..288: mol_type = other RNA
aggcaagcacgcagcaatgcagctcaaaacgcttagcctagccacacccccacgggaaacagcagtgattaacctttagcaataaacgaaagtttaactaagctatactaaccccagggttggtcaatttcgtgccagccacaccccctcgactggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 613 - RNA, 261 nt, Synthetic construct
  source 1..261: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaaccctcgagtgtgtggaggacaccctgaaccccccgctttcaaacaagttttcaaattgtttgaggtcaggatttctcaaactgattcctttctttgcatatgagtatttgaaaataaatattttccc

SEQ ID NO: 614 - RNA, 247 nt, Synthetic construct
  source 1..247: mol_type = other RNA
gtgtgtggaggacaccctgaaccccccgctttcaaacaagttttcaaattgtttgaggtcaggatttctcaaactgattcctttctttgcatatgagtatttgaaaataaatattttcccccctcgagtgtgtggaggacaccctgaaccccccgctttcaaacaagttttcaaattgtttgaggtcaggatttctcaaactgattcctttctttgcatatgagtatttgaaaataaatattttccc

SEQ ID NO: 615 - RNA, 60 nt, Synthetic construct
  source 1..60: mol_type = other RNA
cgtctgcataacttttattatttcttttattaatcaacaaaattttgtttttaacatttc

SEQ ID NO: 616 - RNA, 201 nt, Synthetic construct
  source 1..201: mol_type = other RNA
gtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccttcggggtactcttaacgctgacactcgaaagagcagttcggcaaccc

SEQ ID NO: 617 - RNA, 436 nt, Synthetic construct
  source 1..436: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaaccctcgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 618 - RNA, 273 nt, Synthetic construct
  source 1..273: mol_type = other RNA
agctcgctttcttgctccccaatttctattattccttggaatgaagggaaagtccaactactaaactgtcccttattttgttccaggaagagctagacctatctgccttaaacgagacacgtttattcaccgcaccctcgagctcgctttcttgctgtccaatttctattaaaggttcctttgttccctaagtccaactactaaactgggggatattatgaagggccttgagcatctggattctgcctaataaaaaacatttattttcattgc

SEQ ID NO: 619 - RNA, 71 nt, Synthetic construct
  source 1..71: mol_type = other RNA
aggcaaaaatcaaaatcaatcatcatcacaacatcaacaatcaatcatcaacacatcatcaagacgccacc

SEQ ID NO: 620 - RNA, 113 nt, Synthetic construct
  source 1..113: mol_type = other RNA
aatgtcagaaatattaacaaaaattctcagcattaaagaaaacattgattttcctttcctgactataaaactaattgtgcattatagaaaagtttaaatcacagaatggtatt

SEQ ID NO: 621 - protein, 574 aa, Synthetic construct
MELPILKTNAITTILAAVTLCFASSQNITEEFYQSTCSAVSKGYLSALRTGWYHSVITIELSNIKENKCNGTDAKVKLIKQELDKYKNAVTELQLLMQSTPACNSRARRELPRFMNYTLNNTKNTNVTLSKKRKRRFLGFLLGVGSACASGIAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 622 - protein, 574 aa, Synthetic construct
MELLIHRSSAIFLTLAINALYLTSSQNITEEFYQSTCSAVSRGYFSALRTGWYHSVITIELSNIKETKCNGTDTKVKLIKQELDKYKNAVTELQLLMQNTPACNNRARREAPQYMNYTINTTKNLNVSISKKRKRRFLGFLLGVGSACASGIAVSKVLHLEGEVNKIKNALLSTNKAVVSLSNGVSVLTIKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEILAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSSEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 623 - protein, 574 aa, Synthetic construct
MELPILKTNAITTILAAVTLCFASSQNITEEFYQSTCSAVSKGYLSALRTGWYHCVITIELSNIKENKCNGTDAKVKLIKQELDKYKNAVTELQLLMQSTPAANSRARRELPRFMNYTLNNTKNTNVTLSKKRKRRFLGFLLGVGSAIASGIAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 624 - protein, 574 aa, Synthetic construct
MELLIHRSSAIFLTLAINALYLTSSQNITEEFYQSTCSAVSRGYFSALRTGWYHCVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVCTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSSEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 625 - protein, 544 aa, Synthetic construct
MELPILKTNAITTILAAVTLCFASSQNITEEFYQSTCSAVSKGYLSALRTGWYTSVITIELSNIKENKCNGTDAKVKLIKQELDKYKNAVTELQLLMQSTPACNSRARRELPRFMNYTLNNTKNTNVTLSKKRKRRFLGFLLGVGSACASGIAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELLSAIGGYIPEAPRDGQAYVRKDGEWVLLSTFL

SEQ ID NO: 626 - protein, 544 aa, Synthetic construct
MELLIHRSSAIFLTLAINALYLTSSQNITEEFYQSTCSAVSRGYFSALRTGWYTSVITIELSNIKETKCNGTDTKVKLIKQELDKYKNAVTELQLLMQNTPACNNRARREAPQYMNYTINTTKNLNVSISKKRKRRFLGFLLGVGSACASGIAVSKVLHLEGEVNKIKNALLSTNKAVVSLSNGVSVLTIKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSSEFDASISQVNEKINQSLAFIRRSDELLSAIGGYIPEAPRDGQAYVRKDGEWVLLSTFL

SEQ ID NO: 627 - DNA, 1728 bp, Synthetic construct
  source 1..1728: mol_type = other DNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtaccacagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagagatcctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 628 - DNA, 1728 bp, Synthetic construct
  source 1..1728: mol_type = other DNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtaccacagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagagatcctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

SEQ ID NO: 629 - DNA, 1728 bp, Synthetic construct
  source 1..1728: mol_type = other DNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtaccactgcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgccgccaatagcagagccagacgggaactgcctagattcatgaactacaccctgaacaacaccaagaacaccaacgtgaccctgagcaagaagcggaagcggagattcctgggctttctgctcggagtgggaagcgccattgcctctggaatcgccgtgtctaaagtgctgcacctggaaggcgaagtgaacaagatcaagagcgccctgctgagcaccaacaaggccgtggtgtctctgagcaatggcgtgtccgtgtgcaccagcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 630 - DNA, 1728 bp, Synthetic construct
  source 1..1728: mol_type = other DNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtaccactgcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgccgccaacaaccgggccagaagagaagcccctcagtacatgaactacaccatcaacaccaccaagaacctgaacgtgtccatcagcaagaagcggaagcggcggttcctgggctttctgcttggagtgggaagcgccattgccagcggaatcgccgtgtctaaagtgctgcacctggaaggcgaagtgaacaagatcaagaatgccctgctgagcaccaacaaggccgtggtgtctctgagcaatggcgtgtccgtgtgcacctccaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

SEQ ID NO: 631 - DNA, 1638 bp, Synthetic construct
  source 1..1638: mol_type = other DNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgagcgccatcggcggctatatccctgaggctcctagagatggccaggcctatgtgcggaaggatggcgaatgggtgctgctgtctaccttcctgtgatga

SEQ ID NO: 632 - DNA, 1638 bp, Synthetic construct
  source 1..1638: mol_type = other DNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgagcgccatcggcggctatatccctgaggctcctagagatggccaggcctatgtgcggaaggatggcgaatgggtgctgctgtctaccttcctgtgatga

SEQ ID NO: 633 - RNA, 1728 nt, Synthetic construct
  source 1..1728: mol_type = other RNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtaccacagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagagatcctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 634 - RNA, 1728 nt, Synthetic construct
  source 1..1728: mol_type = other RNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtaccacagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagagatcctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

SEQ ID NO: 635 - RNA, 1728 nt, Synthetic construct
  source 1..1728: mol_type = other RNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtaccactgcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgccgccaatagcagagccagacgggaactgcctagattcatgaactacaccctgaacaacaccaagaacaccaacgtgaccctgagcaagaagcggaagcggagattcctgggctttctgctcggagtgggaagcgccattgcctctggaatcgccgtgtctaaagtgctgcacctggaaggcgaagtgaacaagatcaagagcgccctgctgagcaccaacaaggccgtggtgtctctgagcaatggcgtgtccgtgtgcaccagcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 636 - RNA, 1728 nt, Synthetic construct
  source 1..1728: mol_type = other RNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtaccactgcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgccgccaacaaccgggccagaagagaagcccctcagtacatgaactacaccatcaacaccaccaagaacctgaacgtgtccatcagcaagaagcggaagcggcggttcctgggctttctgcttggagtgggaagcgccattgccagcggaatcgccgtgtctaaagtgctgcacctggaaggcgaagtgaacaagatcaagaatgccctgctgagcaccaacaaggccgtggtgtctctgagcaatggcgtgtccgtgtgcacctccaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

SEQ ID NO: 637 - RNA, 1638 nt, Synthetic construct
  source 1..1638: mol_type = other RNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgcctgcaatagcagagccagacgggaactgcctagattcatgaactacaccctgaacaacaccaagaacaccaacgtgaccctgagcaagaagcggaagcggagattcctgggctttctgctcggagtgggaagcgcctgcgcctctggaatcgccgtgtctaaagtgctgcacctggaaggcgaagtgaacaagatcaagagcgccctgctgagcaccaacaaggccgtggtgtctctgagcaatggcgtgtccgtgctgaccatcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcagcgagttcgatgccagcatctcccaagtgaacgagaagatcaaccagagcctggccttcatcagaaagtccgatgagctgctgagcgccatcggcggctatatccctgaggctcctagagatggccaggcctatgtgcggaaggatggcgaatgggtgctgctgtctaccttcctgtgatga

SEQ ID NO: 638 - RNA, 1638 nt, Synthetic construct
  source 1..1638: mol_type = other RNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgcctgcaacaaccgggccagaagagaagcccctcagtacatgaactacaccatcaacaccaccaagaacctgaacgtgtccatcagcaagaagcggaagcggcggttcctgggctttctgcttggagtgggaagcgcctgcgccagcggaatcgccgtgtctaaagtgctgcacctggaaggcgaagtgaacaagatcaagaatgccctgctgagcaccaacaaggccgtggtgtctctgagcaatggcgtgtccgtgctgaccatcaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcagcgagttcgatgccagcatcagccaagtgaacgagaagatcaaccagagcctggccttcatcaggcggagcgacgaactgctgagcgccatcggcggctatatccctgaggctcctagagatggccaggcctatgtgcggaaggatggcgaatgggtgctgctgtctaccttcctgtgatga

SEQ ID NO: 639 - protein, 539 aa, Human Metapneumovirus
MSWKVVIIFSLLITPQHGLKESYLEESCSTITEGYLSVLRTGWYTNVFTLEVGDVENLTCADGPSLIKTELDLTKSALRELKTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 640 - protein, 539 aa, Human Metapneumovirus
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSNSFDPIRFPEDQFNVALDQVFESIENSQALVDQSNKILNSAEKGNTGFIIVIILIAVLGLTMISVSIIIIIKKTRKPAGAPPELNGVTNGGFIPHS

SEQ ID NO: 641 - protein, 539 aa, Synthetic construct
MSWKVVIIFSLLITPQHGLKESYLEESCSTITEGYLSVLRTGWYTNVFTLEVGDVENLTCADGPSLIKTELDLTKSALRELKTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 642 - protein, 539 aa, Synthetic construct
MSWKVVIIFSLLITPQHGLKESYLEESCSTITEGYLSVLRTGWYTNVFTLEVGDVENLTCADGPSLIKTELDLTKSALRELKTVSADQLAREEQIENPRRRRFVLGAIACGVATAAAVTAGVAIAKCIRLESEVTAIKNCLKKTNECVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPCEKDCETRGDHVFCDTAAGINVAEQSKECNINISTTNYPCKVSCGRHPISMVALSPLGALVACYKGVSCSIGSNRVGIIKQLNKGCSYITNQDADTVTIDNTVYQLSKVEGEQHVIKGRPVSSSFDPVKFPQDQFNVALDQCFENIENSQALVDQSNRILSSAEKGNTGFIIVIILIAVLGSSMILVSIFIIIKKTKKPTGAPPELSGVTNNGFIPHS

SEQ ID NO: 643 - protein, 521 aa, Synthetic construct
MSWKVVIIFSLLITPQHGLKESYLEESCSTITEGYLSVLRTGWYTNVFTLEVGDVENLTCADGPSLIKTELDLTKSALRELKTCSADQGSGGSGATAAAVTAGVAIAKTIRLESEVTAIKNCLKKTNECVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRCMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPECQFNCALDQVFENIENSQALVDQSNRILSSAEKGNTGFIIVIILIAVLGSSMILVSIFIIIKKTKKPTGAPPELSGVTNNGFIPHS

SEQ ID NO: 644 - protein, 539 aa, Synthetic construct
MSWKVMIIISLLITPQHGLKESYLEESCSTITEGYLSVLRTGWYTNVFTLEVGDVENLTCTDGPSLIKTELDLTKSALRELKTVSADQLAREEQIENPRRRRFVLGAIACGVATAAAVTAGIAIAKCIRLESEVNAIKGCLKTTNECVSTLGCGVRVLATAVRELKEFVSKNLTSAINKNKCDIPDLKMAVSFSQFNRRFLNVVRQFSDNAGITPAISKDLMNDAELARAISYMPTSAGQIKLMLENRAMVRRKGFGILIGVYGSSVIYMVQLPIFGVINTPCWIIKAAPSCSEKDGNYACLLREDQGWYCKNAGSTVYYPCEKDCETRGDHVFCDTAAGINVAEQSRECNINISTTNYPCKVSCGRHPISMVALSPLGALVACYKGVSCSIGSNQVGIIKQLPKGCSYITNQDADTVTIDNTVYQLSKVEGEQHVIKGRPVSNSFDPIRFPQDQFNVALDQCFESIENSQALVDQSNKILNSAEKGNTGFIIVIILIAVLGLTMISVSIIIIIKKTRKPAGAPPELNGVTNGGFIPHS

SEQ ID NO: 645 - protein, 521 aa, Synthetic construct
MSWKVMIIISLLITPQHGLKESYLEESCSTITEGYLSVLRTGWYTNVFTLEVGDVENLTCTDGPSLIKTELDLTKSALRELKTCSADQGSGGSGATAAAVTAGIAIAKTIRLESEVNAIKGCLKTTNECVSTLGNGVRVLATAVRELKEFVSKNLTSAINKNKCDIPDLKMAVSFSQFNRRFLNVVRQFSDNAGITPAISLDLMNDAELARAVSYMPTSAGQIKLMLENRCMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSNSFDPIRFPECQFNCALDQVFESIENSQALVDQSNKILNSAEKGNTGFIIVIILIAVLGLTMISVSIIIIIKKTRKPAGAPPELNGVTNGGFIPHS

SEQ ID NO: 646 - protein, 520 aa, Synthetic construct
MSWKVVIIFSLLITPQHGLKESYLEESCSTITEGYLSVLRTGWYTNVFTLEVGDVENLTCADGPSLIKTELDLTKSALRELKTVSADQLAREEQIENPRRRRFVLGAIACGVATAAAVTAGVAIAKCIRLESEVTAIKNCLKKTNECVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPCEKDCETRGDHVFCDTAAGINVAEQSKECNINISTTNYPCKVSCGRHPISMVALSPLGALVACYKGVSCSIGSNRVGIIKQLNKGCSYITNQDADTVTIDNTVYQLSKVEGEQHVIKGRPVSSSFDPVKFPQDQFNVALDQCFENIENSQALVDQSNRILSSAEKGNTSAIGGYIPEAPRDGQAYVRKDGEWVLLSTFL

SEQ ID NO: 647 - protein, 520 aa, Synthetic construct
MSWKVMIIISLLITPQHGLKESYLEESCSTITEGYLSVLRTGWYTNVFTLEVGDVENLTCTDGPSLIKTELDLTKSALRELKTVSADQLAREEQIENPRRRRFVLGAIACGVATAAAVTAGIAIAKCIRLESEVNAIKGCLKTTNECVSTLGCGVRVLATAVRELKEFVSKNLTSAINKNKCDIPDLKMAVSFSQFNRRFLNVVRQFSDNAGITPAISKDLMNDAELARAISYMPTSAGQIKLMLENRAMVRRKGFGILIGVYGSSVIYMVQLPIFGVINTPCWIIKAAPSCSEKDGNYACLLREDQGWYCKNAGSTVYYPCEKDCETRGDHVFCDTAAGINVAEQSRECNINISTTNYPCKVSCGRHPISMVALSPLGALVACYKGVSCSIGSNQVGIIKQLPKGCSYITNQDADTVTIDNTVYQLSKVEGEQHVIKGRPVSNSFDPIRFPQDQFNVALDQCFESIENSQALVDQSNKILNSAEKGNTSAIGGYIPEAPRDGQAYVRKDGEWVLLSTFL

SEQ ID NO: 648 - DNA, 1623 bp, Human Metapneumovirus
  source 1..1623: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaacgccctgaaaaagaccaacgaggccgtgtctaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgaggaccagttcaatgtggccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatga

SEQ ID NO: 649 - DNA, 1623 bp, Synthetic construct
  source 1..1623: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagaagacggagattcgtgctgggagctatcgccctgggagttgctacagctgctgctgtgacagctggcgtggccattgccaagaccatccggctggaaagcgaagtgaccgccatcaagaacgccctgaaaaagaccaacgaggccgtgtctaccctcggcaatggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgaggaccagttcaatgtggccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatga

SEQ ID NO: 650 - DNA, 1623 bp, Synthetic construct
  source 1..1623: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacggcggagattcgtgctgggagctatcgcctgcggagttgctacagctgctgctgtgacagctggcgtggccattgccaagtgcatccggctggaaagcgaagtgaccgccatcaagaactgcctgaaaaagaccaacgagtgcgtgtctaccctcggctgcggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcaaggatctgatgaccgatgccgaactggctagagccatctccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactacccctgcgagaaggactgcgagacaagaggcgaccacgtgttctgtgataccgccgctggaatcaacgtggccgagcagagcaaagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttcccccaggaccagttcaatgtggccctggaccagtgcttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatga

SEQ ID NO: 651 - DNA, 1569 bp, Synthetic construct
  source 1..1569: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaacctgctctgcagatcagggcagcggcggcagcggcgctacagctgctgctgtgacagctggcgtggccattgccaagaccatccggctggaaagcgaagtgaccgccatcaagaactgcctgaaaaagaccaacgagtgcgtgtctaccctcggcaatggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagatgcatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgagtgccagttcaattgcgccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatga

SEQ ID NO: 652 - DNA, 1623 bp, Human Metapneumovirus
  source 1..1623: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatga

SEQ ID NO: 653 - DNA, 1623 bp, Synthetic construct
  source 1..1623: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgcctgcggagttgctacagctgctgctgtgacagccggaatcgccattgccaagtgcatccggctggaaagcgaagtgaacgccatcaagggctgcctgaaaaccaccaacgagtgcgtgtctaccctcggctgcggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcaaggacctgatgaacgacgccgaactggctagagccatctcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactacccctgcgagaaggattgcgagacacggggcgatcacgtgttctgtgatacagccgccggaatcaacgtggccgagcagagcagagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattcccccaggaccagttcaatgtggccctggaccagtgcttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatga

SEQ ID NO: 654 - DNA, 1569 bp, Synthetic construct
  source 1..1569: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaacctgctctgcagatcagggcagcggcggcagcggcgctacagctgctgctgtgacagccggaatcgccattgccaagaccatccggctggaaagcgaagtgaacgccatcaagggctgcctgaaaaccaccaacgagtgcgtgtctaccctcggcaacggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagatgcatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgagtgccagttcaattgcgccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatga

SEQ ID NO: 655 - DNA, 1890 bp, Synthetic construct
  source 1..1890: mol_type = other DNA
aggtgtcagagtttaacttgaagactatttctagggataatatccctgccaccatgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacggcggagattcgtgctgggagctatcgcctgcggagttgctacagctgctgctgtgacagctggcgtggccattgccaagtgcatccggctggaaagcgaagtgaccgccatcaagaactgcctgaaaaagaccaacgagtgcgtgtctaccctcggctgcggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcaaggatctgatgaccgatgccgaactggctagagccatctccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactacccctgcgagaaggactgcgagacaagaggcgaccacgtgttctgtgataccgccgctggaatcaacgtggccgagcagagcaaagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttcccccaggaccagttcaatgtggccctggaccagtgcttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 656 - DNA, 1836 bp, Synthetic construct
  source 1..1836: mol_type = other DNA
aggtgtcagagtttaacttgaagactatttctagggataatatccctgccaccatgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaacctgctctgcagatcagggcagcggcggcagcggcgctacagctgctgctgtgacagctggcgtggccattgccaagaccatccggctggaaagcgaagtgaccgccatcaagaactgcctgaaaaagaccaacgagtgcgtgtctaccctcggcaatggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagatgcatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgagtgccagttcaattgcgccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 657 - DNA, 1890 bp, Synthetic construct
  source 1..1890: mol_type = other DNA
aggtgtcagagtttaacttgaagactatttctagggataatatccctgccaccatgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgcctgcggagttgctacagctgctgctgtgacagccggaatcgccattgccaagtgcatccggctggaaagcgaagtgaacgccatcaagggctgcctgaaaaccaccaacgagtgcgtgtctaccctcggctgcggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcaaggacctgatgaacgacgccgaactggctagagccatctcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactacccctgcgagaaggattgcgagacacggggcgatcacgtgttctgtgatacagccgccggaatcaacgtggccgagcagagcagagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattcccccaggaccagttcaatgtggccctggaccagtgcttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 658 - DNA, 1836 bp, Synthetic construct
  source 1..1836: mol_type = other DNA
aggtgtcagagtttaacttgaagactatttctagggataatatccctgccaccatgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaacctgctctgcagatcagggcagcggcggcagcggcgctacagctgctgctgtgacagccggaatcgccattgccaagaccatccggctggaaagcgaagtgaacgccatcaagggctgcctgaaaaccaccaacgagtgcgtgtctaccctcggcaacggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagatgcatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgagtgccagttcaattgcgccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 659 - DNA, 1566 bp, Synthetic construct
  source 1..1566: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacggcggagattcgtgctgggagctatcgcctgcggagttgctacagctgctgctgtgacagctggcgtggccattgccaagtgcatccggctggaaagcgaagtgaccgccatcaagaactgcctgaaaaagaccaacgagtgcgtgtctaccctcggctgcggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcaaggatctgatgaccgatgccgaactggctagagccatctccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactacccctgcgagaaggactgcgagacaagaggcgaccacgtgttctgtgataccgccgctggaatcaacgtggccgagcagagcaaagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttcccccaggaccagttcaatgtggccctggaccagtgcttcgagaacatcgagaactctcaggctctggtggaccagtccaaccggattctgtctagcgccgagaagggaaacaccagcgccatcggcggctatatccctgaggctcctagagatggccaggcctatgtgcggaaggatggcgaatgggtgctgctgtctaccttcctgtgatga

SEQ ID NO: 660 - DNA, 1566 bp, Synthetic construct
  source 1..1566: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgcctgcggagttgctacagctgctgctgtgacagccggaatcgccattgccaagtgcatccggctggaaagcgaagtgaacgccatcaagggctgcctgaaaaccaccaacgagtgcgtgtctaccctcggctgcggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcaaggacctgatgaacgacgccgaactggctagagccatctcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactacccctgcgagaaggattgcgagacacggggcgatcacgtgttctgtgatacagccgccggaatcaacgtggccgagcagagcagagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattcccccaggaccagttcaatgtggccctggaccagtgcttcgagagcatcgagaatagccaggctctggtggaccagtccaacaagatcctgaactccgccgagaagggcaacaccagcgccatcggcggctatatccctgaggctcctagagatggccaggcctatgtgcggaaggatggcgaatgggtgctgctgtctaccttcctgtgatga

SEQ ID NO: 661 - RNA, 1623 nt, Human Metapneumovirus
  source 1..1623: mol_type = mRNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagaagacggagattcgtgctgggagctatcgccctgggagttgctacagctgctgctgtgacagctggcgtggccattgccaagaccatccggctggaaagcgaagtgaccgccatcaagaacgccctgaaaaagaccaacgaggccgtgtctaccctcggcaatggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgaggaccagttcaatgtggccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatga

SEQ ID NO: 662 - RNA, 1623 nt, Synthetic construct
  source 1..1623: mol_type = other RNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacggcggagattcgtgctgggagctatcgcctgcggagttgctacagctgctgctgtgacagctggcgtggccattgccaagtgcatccggctggaaagcgaagtgaccgccatcaagaactgcctgaaaaagaccaacgagtgcgtgtctaccctcggctgcggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcaaggatctgatgaccgatgccgaactggctagagccatctccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactacccctgcgagaaggactgcgagacaagaggcgaccacgtgttctgtgataccgccgctggaatcaacgtggccgagcagagcaaagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttcccccaggaccagttcaatgtggccctggaccagtgcttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatga

SEQ ID NO: 663 - RNA, 1569 nt, Synthetic construct
  source 1..1569: mol_type = other RNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaacctgctctgcagatcagggcagcggcggcagcggcgctacagctgctgctgtgacagctggcgtggccattgccaagaccatccggctggaaagcgaagtgaccgccatcaagaactgcctgaaaaagaccaacgagtgcgtgtctaccctcggcaatggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagatgcatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgagtgccagttcaattgcgccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatga

SEQ ID NO: 664 - RNA, 1623 nt, Synthetic construct
  source 1..1623: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgcctgcggagttgctacagctgctgctgtgacagccggaatcgccattgccaagtgcatccggctggaaagcgaagtgaacgccatcaagggctgcctgaaaaccaccaacgagtgcgtgtctaccctcggctgcggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcaaggacctgatgaacgacgccgaactggctagagccatctcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactacccctgcgagaaggattgcgagacacggggcgatcacgtgttctgtgatacagccgccggaatcaacgtggccgagcagagcagagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattcccccaggaccagttcaatgtggccctggaccagtgcttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatga

SEQ ID NO: 665 - RNA, 1569 nt, Synthetic construct
  source 1..1569: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaacctgctctgcagatcagggcagcggcggcagcggcgctacagctgctgctgtgacagccggaatcgccattgccaagaccatccggctggaaagcgaagtgaacgccatcaagggctgcctgaaaaccaccaacgagtgcgtgtctaccctcggcaacggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagatgcatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgagtgccagttcaattgcgccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatga

SEQ ID NO: 666 - RNA, 1890 nt, Synthetic construct
  source 1..1890: mol_type = other RNA
aggtgtcagagtttaacttgaagactatttctagggataatatccctgccaccatgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacggcggagattcgtgctgggagctatcgcctgcggagttgctacagctgctgctgtgacagctggcgtggccattgccaagtgcatccggctggaaagcgaagtgaccgccatcaagaactgcctgaaaaagaccaacgagtgcgtgtctaccctcggctgcggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcaaggatctgatgaccgatgccgaactggctagagccatctccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactacccctgcgagaaggactgcgagacaagaggcgaccacgtgttctgtgataccgccgctggaatcaacgtggccgagcagagcaaagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttcccccaggaccagttcaatgtggccctggaccagtgcttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 667 - RNA, 1836 nt, Synthetic construct
  source 1..1836: mol_type = other RNA
aggtgtcagagtttaacttgaagactatttctagggataatatccctgccaccatgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaacctgctctgcagatcagggcagcggcggcagcggcgctacagctgctgctgtgacagctggcgtggccattgccaagaccatccggctggaaagcgaagtgaccgccatcaagaactgcctgaaaaagaccaacgagtgcgtgtctaccctcggcaatggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagatgcatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgagtgccagttcaattgcgccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 668 - RNA, 1890 nt, Synthetic construct
  source 1..1890: mol_type = other RNA
aggtgtcagagtttaacttgaagactatttctagggataatatccctgccaccatgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgcctgcggagttgctacagctgctgctgtgacagccggaatcgccattgccaagtgcatccggctggaaagcgaagtgaacgccatcaagggctgcctgaaaaccaccaacgagtgcgtgtctaccctcggctgcggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcaaggacctgatgaacgacgccgaactggctagagccatctcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactacccctgcgagaaggattgcgagacacggggcgatcacgtgttctgtgatacagccgccggaatcaacgtggccgagcagagcagagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattcccccaggaccagttcaatgtggccctggaccagtgcttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 669 - RNA, 1836 nt, Synthetic construct
  source 1..1836: mol_type = other RNA
aggtgtcagagtttaacttgaagactatttctagggataatatccctgccaccatgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaacctgctctgcagatcagggcagcggcggcagcggcgctacagctgctgctgtgacagccggaatcgccattgccaagaccatccggctggaaagcgaagtgaacgccatcaagggctgcctgaaaaccaccaacgagtgcgtgtctaccctcggcaacggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagatgcatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgagtgccagttcaattgcgccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 670 - RNA, 1566 nt, Synthetic construct
  source 1..1566: mol_type = other RNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacggcggagattcgtgctgggagctatcgcctgcggagttgctacagctgctgctgtgacagctggcgtggccattgccaagtgcatccggctggaaagcgaagtgaccgccatcaagaactgcctgaaaaagaccaacgagtgcgtgtctaccctcggctgcggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcaaggatctgatgaccgatgccgaactggctagagccatctccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactacccctgcgagaaggactgcgagacaagaggcgaccacgtgttctgtgataccgccgctggaatcaacgtggccgagcagagcaaagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttcccccaggaccagttcaatgtggccctggaccagtgcttcgagaacatcgagaactctcaggctctggtggaccagtccaaccggattctgtctagcgccgagaagggaaacaccagcgccatcggcggctatatccctgaggctcctagagatggccaggcctatgtgcggaaggatggcgaatgggtgctgctgtctaccttcctgtgatga

SEQ ID NO: 671 - RNA, 1566 nt, Synthetic construct
  source 1..1566: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgcctgcggagttgctacagctgctgctgtgacagccggaatcgccattgccaagtgcatccggctggaaagcgaagtgaacgccatcaagggctgcctgaaaaccaccaacgagtgcgtgtctaccctcggctgcggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcaaggacctgatgaacgacgccgaactggctagagccatctcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactacccctgcgagaaggattgcgagacacggggcgatcacgtgttctgtgatacagccgccggaatcaacgtggccgagcagagcagagagtgcaacatcaacatcagcaccacaaactacccctgcaaggtgtcctgcggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattcccccaggaccagttcaatgtggccctggaccagtgcttcgagagcatcgagaatagccaggctctggtggaccagtccaacaagatcctgaactccgccgagaagggcaacaccagcgccatcggcggctatatccctgaggctcctagagatggccaggcctatgtgcggaaggatggcgaatgggtgctgctgtctaccttcctgtgatga

SEQ ID NO: 672 - protein, 555 aa, Parainfluenza virus type 1
MQSSEILLLVYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGGWHNKESTQIIIIIIVCILIIIICGILYYLYRVRRLLIMINSTNNSPINAYTLESRMKNPYMGNHSN

SEQ ID NO: 673 - protein, 555 aa, Synthetic construct
MQSSEILLLVYSSLLLSSSLCQIPVDKLSNVGVIINEGKLLKIAGSYESRYIVLSLVPSIDLQDGCGTTQIIQYKNLLNRLLIPLKDALDLCESLITITNDTTVTNDNPQTRGSGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKLRAIISAVGGWHNKESTQIIIIIIVCILIIIICGILYYLYRVRRLLIMINSTNNSPINAYTLESRMKNPYMGNHSN

SEQ ID NO: 674 - protein, 555 aa, Synthetic construct
MQSSEILLLVYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRGSGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKARAIISAVGGWHNKESTQIIIIIIVCILIIIICGILYYLYRVRRLLIMINSTNNSPINAYTLESRMKNPYMGNHSN

SEQ ID NO: 675 - protein, 511 aa, Synthetic construct
MQSSEILLLVYSSLLLSSSLCQIPVDKLSNVGVIINEGKLLKIAGSYESRYIVLSLVPSIDLQDGCGTTQIIQYKNLLNRLLIPLKDALDLCESLITITNDTTVTNDNPQTRGSGAVIGTIALGVATAAQITACIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKLSAIGGYIPEAPRDGQAYVRKDGEWVLLSTFL

SEQ ID NO: 676 - protein, 511 aa, Synthetic construct
MQSSEILLLVYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRGSGAVIGTIALGVATAAQITAAIALAEAREARKDIALIKDSIVKTHNSVEFIQRGIGEQIIALKTLQDFVNDEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASLTNFLEELKTELMKASAIGGYIPEAPRDGQAYVRKDGEWVLLSTFL

SEQ ID NO: 677 - DNA, 1671 bp, Parainfluenza virus type 1
  source 1..1671: mol_type = other DNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagattcttcggcgccgtgatcggaacaatcgccctgggagttgctaccgccgctcagatcacagccggaattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcgccaccaactttctggaagaaagcaagaccgaactcatgaaggccagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaactgatga

SEQ ID NO: 678 - DNA, 1671 bp, Synthetic construct
  source 1..1671: mol_type = other DNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgtgcgagagcctgatcaccatcaccaacgacaccaccgtgaccaacgataaccctcagaccagaggcagcggcgccgtgatcggaacaatcgccctgggagttgctaccgccgctcagatcacagcctgcattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaactgatga

SEQ ID NO: 679 - DNA, 1671 bp, Synthetic construct
  source 1..1671: mol_type = other DNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggcagcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaaggccagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaactgatga

SEQ ID NO: 680 - DNA, 1935 bp, Synthetic construct
  source 1..1935: mol_type = other DNA
aggaggactggtcgaacctgcatagtgatcataaggtcagcatagccaccatgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgtgcgagagcctgatcaccatcaccaacgacaccaccgtgaccaacgataaccctcagaccagaggcagcggcgccgtgatcggaacaatcgccctgggagttgctaccgccgctcagatcacagcctgcattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaactgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 681 - DNA, 1935 bp, Synthetic construct
  source 1..1935: mol_type = other DNA
aggaggactggtcgaacctgcatagtgatcataaggtcagcatagccaccatgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggcagcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaaggccagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaactgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 682 - DNA, 1539 bp, Synthetic construct
  source 1..1539: mol_type = other DNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgtgcgagagcctgatcaccatcaccaacgacaccaccgtgaccaacgataaccctcagaccagaggcagcggcgccgtgatcggaacaatcgccctgggagttgctaccgccgctcagatcacagcctgcattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagcgccatcggcggctatatccctgaggctcctagagatggccaggcctatgtgcggaaggatggcgaatgggtgctgctgtctaccttcctgtgatga

SEQ ID NO: 683 - DNA, 1539 bp, Synthetic construct
  source 1..1539: mol_type = other DNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggcagcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaaggccagcgccatcggcggctatatccctgaggctcctagagatggccaggcctatgtgcggaaggatggcgaatgggtgctgctgtctaccttcctgtgatga

SEQ ID NO: 684 - RNA, 1671 nt, Synthetic construct
  source 1..1671: mol_type = other RNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgtgcgagagcctgatcaccatcaccaacgacaccaccgtgaccaacgataaccctcagaccagaggcagcggcgccgtgatcggaacaatcgccctgggagttgctaccgccgctcagatcacagcctgcattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaactgatga

SEQ ID NO: 685 - RNA, 1671 nt, Synthetic construct
  source 1..1671: mol_type = other RNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggcagcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaaggccagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaactgatga

SEQ ID NO: 686 - RNA, 1935 nt, Synthetic construct
  source 1..1935: mol_type = other RNA
aggaggactggtcgaacctgcatagtgatcataaggtcagcatagccaccatgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgtgcgagagcctgatcaccatcaccaacgacaccaccgtgaccaacgataaccctcagaccagaggcagcggcgccgtgatcggaacaatcgccctgggagttgctaccgccgctcagatcacagcctgcattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaactgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 687 - RNA, 1935 nt, Synthetic construct
  source 1..1935: mol_type = other RNA
aggaggactggtcgaacctgcatagtgatcataaggtcagcatagccaccatgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggcagcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaaggccagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaactgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 688 - RNA, 1539 nt, Synthetic construct
  source 1..1539: mol_type = other RNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgtgcgagagcctgatcaccatcaccaacgacaccaccgtgaccaacgataaccctcagaccagaggcagcggcgccgtgatcggaacaatcgccctgggagttgctaccgccgctcagatcacagcctgcattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaagctgagcgccatcggcggctatatccctgaggctcctagagatggccaggcctatgtgcggaaggatggcgaatgggtgctgctgtctaccttcctgtgatga

SEQ ID NO: 689 - RNA, 1539 nt, Synthetic construct
  source 1..1539: mol_type = other RNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagaggcagcggcgccgtgatcggaacaatcgccctgggagttgctaccgccgctcagatcacagccgccattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcctgaccaactttctggaagaactgaagaccgaactcatgaaggccagcgccatcggcggctatatccctgaggctcctagagatggccaggcctatgtgcggaaggatggcgaatgggtgctgctgtctaccttcctgtgatga

SEQ ID NO: 690 - protein, 539 aa, Parainfluenza virus type 3
MLISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSWHQSSTTIIVILIMMIILFIINITIITIAIKYYRIQKRNRVDQNDKPYVLTNK

SEQ ID NO: 691 - protein, 539 aa, Synthetic construct
MLISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSWHQSSTTIIVILIMMIILFIINITIITIAIKYYRIQKRNRVDQNDKPYVLTNK

SEQ ID NO: 692 - protein, 539 aa, Synthetic construct
MLISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKLDSIGSWHQSSTTIIVILIMMIILFIINITIITIAIKYYRIQKRNRVDQNDKPYVLTNK

SEQ ID NO: 693 - protein, 512 aa, Synthetic construct
MLISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIGGYIPEAPRDGQAYVRKDGEWVLLSTFL

SEQ ID NO: 694 - protein, 512 aa, Synthetic construct
MLISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQCVQSSVGNLICAIKSVQDYVNKEIVPSIARLGCEAAGLQLGIALTQHYSCLTNIFGDNIGSLQEKGIKLQGIASCYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKLKSDLEELKEWIRRSNQKLSAIGGYIPEAPRDGQAYVRKDGEWVLLSTFL

SEQ ID NO: 695 - DNA, 1623 bp, Parainfluenza virus type 3
  source 1..1623: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcagggaatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatga

SEQ ID NO: 696 - DNA, 1623 bp, Synthetic construct
  source 1..1623: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatga

SEQ ID NO: 697 - DNA, 1623 bp, Synthetic construct
  source 1..1623: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatga

SEQ ID NO: 698 - DNA, 1887 bp, Synthetic construct
  source 1..1887: mol_type = other DNA
aggaggactgggcgaacctgcatagtgatcataaggtcatcatagccaccatgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 699 - DNA, 1887 bp, Synthetic construct
  source 1..1887: mol_type = other DNA
aggaggactgggcgaacctgcatagtgatcataaggtcatcatagccaccatgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 700 - DNA, 1890 bp, Synthetic construct
  source 1..1890: mol_type = other DNA
aggtgtcagagtttaacttgaagactatttctagggataatatccctgccaccatgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 701 - DNA, 1890 bp, Synthetic construct
  source 1..1890: mol_type = other DNA
aggtgtcagagtttaacttgaagactatttctagggataatatccctgccaccatgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 702 - DNA, 1542 bp, Synthetic construct
  source 1..1542: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctgagcgccatcggcggctatatccctgaggctcctagagatggccaggcctatgtgcggaaggatggcgaatgggtgctgctgtctaccttcctgtgatga

SEQ ID NO: 703 - DNA, 1542 bp, Synthetic construct
  source 1..1542: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctgagcgccatcggcggctatatccctgaggctcctagagatggccaggcctatgtgcggaaggatggcgaatgggtgctgctgtctaccttcctgtgatga

SEQ ID NO: 704 - RNA, 1623 nt, Synthetic construct
  source 1..1623: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatga

SEQ ID NO: 705 - RNA, 1623 nt, Synthetic construct
  source 1..1623: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatga

SEQ ID NO: 706 - RNA, 1887 nt, Synthetic construct
  source 1..1887: mol_type = other RNA
aggaggactgggcgaacctgcatagtgatcataaggtcatcatagccaccatgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 707 - RNA, 1887 nt, Synthetic construct
  source 1..1887: mol_type = other RNA
aggaggactgggcgaacctgcatagtgatcataaggtcatcatagccaccatgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 708 - RNA, 1890 nt, Synthetic construct
  source 1..1890: mol_type = other RNA
aggtgtcagagtttaacttgaagactatttctagggataatatccctgccaccatgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 709 - RNA, 1890 nt, Synthetic construct
  source 1..1890: mol_type = other RNA
aggtgtcagagtttaacttgaagactatttctagggataatatccctgccaccatgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 710 - RNA, 1542 nt, Synthetic construct
  source 1..1542: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctgagcgccatcggcggctatatccctgaggctcctagagatggccaggcctatgtgcggaaggatggcgaatgggtgctgctgtctaccttcctgtgatga

SEQ ID NO: 711 - RNA, 1542 nt, Synthetic construct
  source 1..1542: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtgcgtgcaaagcagcgtgggcaatctgatctgcgccattaagagcgtgcaggactacgtgaacaaagagatcgtcccctctatcgccagactgggatgtgaagctgccggactgcagctgggaattgccctgacacagcactacagctgcctgaccaacatcttcggcgacaacatcggcagcctgcaagagaagggcattaagctgcagggaatcgccagctgctaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaagctcaagagcgacctggaagaactgaaagagtggatccggcggagcaaccagaagctgagcgccatcggcggctatatccctgaggctcctagagatggccaggcctatgtgcggaaggatggcgaatgggtgctgctgtctaccttcctgtgatga

SEQ ID NO: 712 - protein, 539 aa, Synthetic construct
MLISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVCSSVGNCIVAIKSVQDYVNKEIVPSIARLGCEAAGLQLGIALTQHYSELTNCFGDNIGSLQEKGIKLQCIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKVKSDLEESKEWYRRSNQKLDSIGSWHQSSTTIIVILIMMIILFIINITIITIAIKYYRIQKRNRVDQNDKPYVLTNK

SEQ ID NO: 713 - DNA, 1623 bp, Synthetic construct
  source 1..1623: mol_type = other DNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgtgcagcagcgtgggcaattgcatcgtggccattaagagcgtgcaggactacgtgaacaaagagatcgtcccctctatcgccagactgggatgtgaagctgccggactgcagctgggaattgccctgacacagcactacagcgagctgaccaactgcttcggcgacaacatcggcagcctgcaagagaagggcattaagctgcagtgcatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggtcaagagcgacctggaagaaagcaaagagtggtaccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatga

SEQ ID NO: 714 - RNA, 1623 nt, Synthetic construct
  source 1..1623: mol_type = other RNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgtgcagcagcgtgggcaattgcatcgtggccattaagagcgtgcaggactacgtgaacaaagagatcgtcccctctatcgccagactgggatgtgaagctgccggactgcagctgggaattgccctgacacagcactacagcgagctgaccaactgcttcggcgacaacatcggcagcctgcaagagaagggcattaagctgcagtgcatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggtcaagagcgacctggaagaaagcaaagagtggtaccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatga

SEQ ID NO: 715 - protein, 502 aa, Synthetic construct
MSWKVVIIFSLLITPQHGLKESYLEESCSTITEGYLSVLRTGWYTNVFTLEVGDVENLTCADGPSLIKTELDLTKSALRELKTCSADQGSGGSGATAAAVTAGVAIAKTIRLESEVTAIKNCLKKTNECVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRCMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPECQFNCALDQVFENIENSQALVDQSNRILSSAEKGNTSAIGGYIPEAPRDGQAYVRKDGEWVLLSTFL

SEQ ID NO: 716 - protein, 502 aa, Synthetic construct
MSWKVMIIISLLITPQHGLKESYLEESCSTITEGYLSVLRTGWYTNVFTLEVGDVENLTCTDGPSLIKTELDLTKSALRELKTCSADQGSGGSGATAAAVTAGIAIAKTIRLESEVNAIKGCLKTTNECVSTLGNGVRVLATAVRELKEFVSKNLTSAINKNKCDIPDLKMAVSFSQFNRRFLNVVRQFSDNAGITPAISLDLMNDAELARAVSYMPTSAGQIKLMLENRCMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSNSFDPIRFPECQFNCALDQVFESIENSQALVDQSNKILNSAEKGNTSAIGGYIPEAPRDGQAYVRKDGEWVLLSTFL

SEQ ID NO: 717 - DNA, 1512 bp, Synthetic construct
  source 1..1512: mol_type = other DNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaacctgctctgcagatcagggcagcggcggcagcggcgctacagctgctgctgtgacagctggcgtggccattgccaagaccatccggctggaaagcgaagtgaccgccatcaagaactgcctgaaaaagaccaacgagtgcgtgtctaccctcggcaatggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagatgcatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgagtgccagttcaattgcgccctggaccaggtgttcgagaacatcgagaactctcaggctctggtggaccagtccaaccggattctgtctagcgccgagaagggaaacaccagcgccatcggcggctatatccctgaggctcctagagatggccaggcctatgtgcggaaggatggcgaatgggtgctgctgtctaccttcctgtgatga

SEQ ID NO: 718 - DNA, 1512 bp, Synthetic construct
  source 1..1512: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaacctgctctgcagatcagggcagcggcggcagcggcgctacagctgctgctgtgacagccggaatcgccattgccaagaccatccggctggaaagcgaagtgaacgccatcaagggctgcctgaaaaccaccaacgagtgcgtgtctaccctcggcaacggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagatgcatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgagtgccagttcaattgcgccctggaccaggtgttcgagagcatcgagaatagccaggctctggtggaccagtccaacaagatcctgaactccgccgagaagggcaacaccagcgccatcggcggctatatccctgaggctcctagagatggccaggcctatgtgcggaaggatggcgaatgggtgctgctgtctaccttcctgtgatga

SEQ ID NO: 719 - RNA, 1512 nt, Synthetic construct
  source 1..1512: mol_type = other RNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaacctgctctgcagatcagggcagcggcggcagcggcgctacagctgctgctgtgacagctggcgtggccattgccaagaccatccggctggaaagcgaagtgaccgccatcaagaactgcctgaaaaagaccaacgagtgcgtgtctaccctcggcaatggcgttagagtgctggccacagccgtccgcgagctgaaggatttcgtgtccaagaacctgaccagggccatcaacaagaacaagtgcgacatccccgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagatgcatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgagtgccagttcaattgcgccctggaccaggtgttcgagaacatcgagaactctcaggctctggtggaccagtccaaccggattctgtctagcgccgagaagggaaacaccagcgccatcggcggctatatccctgaggctcctagagatggccaggcctatgtgcggaaggatggcgaatgggtgctgctgtctaccttcctgtgatga

SEQ ID NO: 720 - RNA, 1512 nt, Synthetic construct
  source 1..1512: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaacctgctctgcagatcagggcagcggcggcagcggcgctacagctgctgctgtgacagccggaatcgccattgccaagaccatccggctggaaagcgaagtgaacgccatcaagggctgcctgaaaaccaccaacgagtgcgtgtctaccctcggcaacggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagatgcatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgagtgccagttcaattgcgccctggaccaggtgttcgagagcatcgagaatagccaggctctggtggaccagtccaacaagatcctgaactccgccgagaagggcaacaccagcgccatcggcggctatatccctgaggctcctagagatggccaggcctatgtgcggaaggatggcgaatgggtgctgctgtctaccttcctgtgatga

SEQ ID NO: 721 - protein, 539 aa, Synthetic construct
MSWKVMIIISLLITPQHGLKESYLEESCSTITEGYLSVLRTGWYTNVFTLEVGDVENLTCTDGPSLIKTELDLTKSALRELKTVSADQLAREEQIENPRRRRFVLGAIALGVATAAAVTAGIAIAKTIRLESEVNAIKGALKTTNEAVSTLGNGVRVLATAVRELKEFVSKNLTSAINKNKCDIPDLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNQVGIIKQLPKGCSYITNQDADTVTIDNTVYQLSKVEGEQHVIKGRPVSNSFDPIRFPEDQFNVALDQVFESIENSQALVDQSNKILNSAEKGNTGFIIVIILIAVLGLTMISVSIIIIIKKTRKPAGAPPELNGVTNGGFIPHS

SEQ ID NO: 722 - DNA, 1623 bp, Synthetic construct
  source 1..1623: mol_type = other DNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatga

SEQ ID NO: 723 - RNA, 1623 nt, Synthetic construct
  source 1..1623: mol_type = other RNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacggcggagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattcccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatga

SEQ ID NO: 724 - protein, 572 aa, Parainfluenza virus type 3
MEYWKHTNHGKDAGNELETSTATHGNKLTNKITYILWTITLVLLSIVFIIVLTNSIKSEKARESLLQDINNEFMEVTEKIQVASDNTNDLIQSGVNTRLLTIQSHVQNYIPISLTQQISDLRKFISEITIRNDNQEVPPQRITHDVGIKPLNPDDFWRCTSGLPSLMKTPKIRLMPGPGLLAMPTTVDGCVRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENAICNTTGCPGKTQRDCNQASHSPWFSDRRMVNSIIVVDKGLNSVPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNKTLSAGYTTTSCITHYNKGYCFHIVEINHKSLNTFQPMLFKTEIPKSCS

SEQ ID NO: 725 - protein, 542 aa, Synthetic construct
MEYWKHTNHGKDAGNELETSTATHGNKLTNKITYILWTITLVLLSIVFIIVLTNSIKSDLIQSGVNTRLLTIQSHVQNYIPISLTQQISDLRKFISEITIRNDNQEVPPQRITHDVGIKPLNPDDFWRCTSGLPSLMKTPKIRLMPGPGLLAMPTTVDGCVRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENAICNTTGCPGKTQRDCNQASHSPWFSDRRMVNSIIVVDKGLNSVPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNKTLSAGYTTTSCITHYNKGYCFHIVEINHKSLNTFQPMLFKTEIPKSCS

SEQ ID NO: 726 - protein, 500 aa, Synthetic construct
MEYWKHTNHGKDAGNELETSTATHGNKLTNKITYILWTITLVLLSIVFIIVLTNSIKSRNDNQEVPPQRITHDVGIKPLNPDDFWRCTSGLPSLMKTPKIRLMPGPGLLAMPTTVDGCVRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENAICNTTGCPGKTQRDCNQASHSPWFSDRRMVNSIIVVDKGLNSVPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNKTLSAGYTTTSCITHYNKGYCFHIVEINHKSLNTFQPMLFKTEIPKSCS

SEQ ID NO: 727 - DNA, 1722 bp, Parainfluenza virus type 3
  source 1..1722: mol_type = other DNA
atggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgagaaggccagagagagcctgctgcaggacatcaacaacgagttcatggaagtgaccgagaagatccaggtggccagcgacaacaccaacgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatga

SEQ ID NO: 728 - DNA, 1632 bp, Synthetic construct
  source 1..1632: mol_type = other DNA
atggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatga

SEQ ID NO: 729 - DNA, 1506 bp, Synthetic construct
  source 1..1506: mol_type = other DNA
atggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatga

SEQ ID NO: 730 - DNA, 1986 bp, Synthetic construct
  source 1..1986: mol_type = other DNA
aggaggactgggcgaacctgcatagtgatcataaggtcatcatagccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgagaaggccagagagagcctgctgcaggacatcaacaacgagttcatggaagtgaccgagaagatccaggtggccagcgacaacaccaacgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 731 - DNA, 1896 bp, Synthetic construct
  source 1..1896: mol_type = other DNA
aggaggactgggcgaacctgcatagtgatcataaggtcatcatagccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 732 - DNA, 1770 bp, Synthetic construct
  source 1..1770: mol_type = other DNA
aggaggactgggcgaacctgcatagtgatcataaggtcatcatagccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 733 - DNA, 1989 bp, Synthetic construct
  source 1..1989: mol_type = other DNA
aggtgtcagagtttaacttgaagactatttctagggataatatccctgccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgagaaggccagagagagcctgctgcaggacatcaacaacgagttcatggaagtgaccgagaagatccaggtggccagcgacaacaccaacgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 734 - DNA, 1899 bp, Synthetic construct
  source 1..1899: mol_type = other DNA
aggtgtcagagtttaacttgaagactatttctagggataatatccctgccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 735 - DNA, 1773 bp, Synthetic construct
  source 1..1773: mol_type = other DNA
aggtgtcagagtttaacttgaagactatttctagggataatatccctgccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 736 - RNA, 1722 nt, Parainfluenza virus type 3
  source 1..1722: mol_type = mRNA
atggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgagaaggccagagagagcctgctgcaggacatcaacaacgagttcatggaagtgaccgagaagatccaggtggccagcgacaacaccaacgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatga

SEQ ID NO: 737 - RNA, 1632 nt, Synthetic construct
  source 1..1632: mol_type = other RNA
atggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatga

SEQ ID NO: 738 - RNA, 1506 nt, Synthetic construct
  source 1..1506: mol_type = other RNA
atggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatga

SEQ ID NO: 739 - RNA, 1986 nt, Synthetic construct
  source 1..1986: mol_type = other RNA
aggaggactgggcgaacctgcatagtgatcataaggtcatcatagccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgagaaggccagagagagcctgctgcaggacatcaacaacgagttcatggaagtgaccgagaagatccaggtggccagcgacaacaccaacgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 740 - RNA, 1896 nt, Synthetic construct
  source 1..1896: mol_type = other RNA
aggaggactgggcgaacctgcatagtgatcataaggtcatcatagccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 741 - RNA, 1770 nt, Synthetic construct
  source 1..1770: mol_type = other RNA
aggaggactgggcgaacctgcatagtgatcataaggtcatcatagccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 742 - RNA, 1989 nt, Synthetic construct
  source 1..1989: mol_type = other RNA
aggtgtcagagtttaacttgaagactatttctagggataatatccctgccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgagaaggccagagagagcctgctgcaggacatcaacaacgagttcatggaagtgaccgagaagatccaggtggccagcgacaacaccaacgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 743 - RNA, 1899 nt, Synthetic construct
  source 1..1899: mol_type = other RNA
aggtgtcagagtttaacttgaagactatttctagggataatatccctgccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagcgacctgattcagagcggcgtgaacacccggctgctgacaattcagagccacgtgcagaactatatccccatcagcctgacacagcagatcagcgacctgcggaagttcatcagcgagatcaccatccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 744 - RNA, 1773 nt, Synthetic construct
  source 1..1773: mol_type = other RNA
aggtgtcagagtttaacttgaagactatttctagggataatatccctgccaccatggaatactggaagcacaccaaccacggcaaggacgccggcaatgagctggaaacaagcacagctacccacggcaacaagctgaccaacaagatcacctacatcctgtggaccatcacactggtgctgctgagcatcgtgttcatcatcgtgctgaccaatagcatcaagagccggaacgacaatcaagaggtgccaccacagcggatcacccacgatgtgggaatcaagcctctgaaccccgacgacttctggcggtgtacatctggcctgcctagcctgatgaagacccctaagatccggctgatgcctggacctggactgctggccatgcctacaacagtggatggctgtgtgcggacccctagcctggtcatcaacgatctgatctacgcctacaccagcaacctgatcaccagaggctgccaggatatcggcaagagctaccaggtgctgcagatcggcatcatcaccgtgaacagcgatctggtgcccgacctgaatcctcggatcagccacaccttcaacatcaacgacaaccggaagtcctgttctctggccctgctgaacaccgacgtgtaccagctgtgtagcacccctaaggtggacgagagaagcgactatgccagcagcggcatcgaggatatcgtgctggacatcgtgaactacgacggcagcatcagcaccaccagattcaagaacaacaacatcagcttcgaccagccttacgccgcactgtaccctagtgttggccctggcatctactacaagggcaagatcatcttcctcggctacggcggcctggaacaccccatcaatgagaacgccatctgcaacaccaccggctgtcctggcaagacccagagagactgcaatcaggccagccactctccatggttcagcgacagacggatggtcaactctatcatcgtggtggacaagggcctgaacagcgtgcccaagctgaaagtgtggacaatcagcatgcggcagaactactggggcagcgaaggcagactgctgctgctgggaaacaagatctacatctacacccggtccaccagctggcacagcaaactgcagctgggaatcatcgacatcaccgactacagcgacatccggatcaagtggacctggcacaacgtgctgagcagacccggcaacaatgagtgtccttggggccactcttgccccgatggatgtatcaccggcgtgtacaccgacgcctatccactgaatcctaccggctccatcgtgtccagcgtgatcctggacagccagaaaagcagagtgaaccccgtgatcacatacagcaccgccaccgagagagtgaacgagctggccatcagaaacaagaccctgagcgccggctacaccaccacaagctgcatcacacactacaacaagggctactgcttccacatcgtcgagatcaaccacaagtccctgaacaccttccagcctatgctgttcaagacagagatccccaagagctgctcctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 745 - protein, 572 aa, Parainfluenza virus type 3
MEYWKHTNHGKDAGNELETSMATHGNKITNNIIYILWTIILVLLSIVFIIVLTNSIKSEKTHESLLRDINNEFMEITEKIQMASDNTNDLIQSGVNTRLLTIQSHVQNYIPISLTQQMSDLRKFISEIIIRNDNQEVLPQRITHDVGIKPLNPDDFWRCTSGLPSLMKTPKIRLMPGPGLLAMPTTVDGCIRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSTPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNRTLSAGYTTTSCITHYNKGYCFHIVEINHKSLNTFQPMLFKTEIPKSCS

SEQ ID NO: 746 - protein, 572 aa, Parainfluenza virus type 3
MEYWKHTNHGKDVGNELETSMATHGNKLTNKIIYILWTIILVLLSIVFIIVLINSIKNEKARESLLQDINNEFMEITEKIQMASDNTNDLIQSGVNTRLLTIQSHVQNYIPISLTQQMSDLRKFISEIVIRNDNQEVLPQRITHDVGIKPLNPDDFWRCTSGLPSLMKTPKIRLMPGPGLLAMPTTVDGCIRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSIPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNRTLSAGYTTTSCITHYNKGYCFHIVEINHKGLNTFQPMLFKTEVPKSCS

SEQ ID NO: 747 - protein, 572 aa, Parainfluenza virus type 3
MEYWKHTNHGKDAGNELETSMATHGNKLTNKIIYILWTIILVLLSIVFIIVLTNSIKSEKAHESLLRDINNEFIGITEKIQMASDNTNDLIQSGVNTRLLTIQSHVQNYIPISLTQQMSDLRKFISEITIRNDNQEVLPQRITHDVGIKPLNPDDFWRCTSGLPSLMKTPKIRLMPGPGLLAMPTTVDGCIRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPIDENVICNTTGCPGKTQRDCNQASHSPWFSDRRMVNSIIVVDKGLNSTPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNRTLSAGYTTTSCITHYNKGYCFHIVEINHKSSNTFQPMLFKTEIPKSCS

SEQ ID NO: 748 - protein, 572 aa, Parainfluenza virus type 3
MEYWKHTNHGKDAGNELETSMATNGNKLTNKITYILWTIILVLLSIVFIIVLINSIKSEKAHESLLQDINNEFMEITEKIQMASDNTNDLIQSGVNTRLLTIQSHVQNYIPISLTQQMSDLRKFISEITIRNDNQEVLPQRITHDVGIKPLNPDDFWRCTSGLPSLMKTPKIRLMPGPGLLAMPTTVDGCIRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSIPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNRTLSAGYTTTSCITHYNKGYCFHIVEINQKSLNTLQPMLFKTEVPKSCS

SEQ ID NO: 749 - protein, 572 aa, Parainfluenza virus type 3
MEYWKHTNHGKDAGNELETSMATHGNKLTNKIIYILWTIILVLLSIVFIIVLTNSIKSEKTHESLLRDINNEFMEITEKIQMASDNTNDLIQSGVNTRLLTIQSHVQNYIPISLTQQMSDLRKFISEIIIRNDNQEVLPQRITHDVGIKPLNPDDFWRCTSGLPSLMKTPKIRLMPGPGLLAMPTTVDGCIRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSTPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNRTLSAGYTTTSCITHYNKGYCFHIVEINHKSSNTFQPMLFKTEIPKSCS

SEQ ID NO: 750 - protein, 575 aa, Parainfluenza virus type 1
MAEKGKTISSYWSTTRNDNSTVNTHINTPAGRTYIWLLIATTMHAALSLIIMILCIDLIIKQDTCMKTNIMTVSSVNESAKTIKETITELIRQEVISRTINIQSSVQSGIPILLNKQSRDLTQLIEKSCNKQELAQICENTIAIHHADGITPLDPHDFWRCPVGEPLLSNNPNISLLPGPSLLSGSTTISGCVRLPSLSIGDAIYAYSSNLITQGCADIGKSYQVLQLGYISLNSDMYPDLNPVISHTYDINDNRKSCSVIAAGTRGYQLCSLPTVNETTDYSSEGIEDLVFDILDLKGKTKSHRYKNEDITFDHPFSAMYPSVGSGIKIENTLVFLGYGGLTTPLQGNTKCVIKSCPNVNQSVCNDALKITWLKKRQVVNVLIHINNYLSDRPKIVVETIPITQNYLGAEGRLLKLGKKIYIYTRSSGWHSNLQIGSIDINKPMTINWAPHKVLSRPGNPDCNWFNKCPRECISGVYTDAYPLSPDAFNVATTTLYANTSRVNPTIMYSSTSKIINMLRLKNGQLEAAYTTTSCITHFGKGYCFHIVEINQTSLNTLQPMLFKTSIPKICKVTS

SEQ ID NO: 751 - protein, 575 aa, Parainfluenza virus type 1
MAEKGKTNSSYWSTTRNDNSTVNTYIDTPAGKTHIWLLIATTMHTILSFIIMILCIDLIIKQDTCMKTNIITISSMNESAKTIKETITELIRQEVISRTINIQSSVQSGIPILLNKQSRDLTQLIEKSCNRQELAQICENTNAIHHADGISPLDPHDFWRCPVGEPLLSDNPNISLLPGPSLLSGSTTISGCVRLPSLSIGDAIYAYSSNLITQGCADIGKSYQVLQLGYISLNSDMYPDLNPVISHTYDINDNRKSCSVIAAGTRGYQLCSLPTVNETTDYSSEGIEDLVFDILDLKGKTKSHRYKNEDITFDHPFSAMYPSVGSGIKIENTLIFLGYGGLTTPLQGDTKCVTNRCANVNQSVCNDALKITWLKKRQVVNVLIRINNYLSDRPKIVVETIPITQNYLGAEGRLLKLGKKIYIYTRSSGWHSHLQIGSLDINNPMTIKWAPHEVLSRPGNQDCNWYNRCPRECISGVYTDAYPLSPDAVNVATTTLYANTSRVNPTIMYSNTSEIINMLRLKNVQLEAAYTTTSCITHFGKGYCFHIVEINQTSLNTLQPMLFKTSIPKICKITS

SEQ ID NO: 752 - protein, 575 aa, Parainfluenza virus type 1
MAEKGKTNSSYWSTTRNDNSTVNTYIDTPAGKTHIWLLIATTMHTILSFIIMILCIDLIIKQDTCMKTNIMTVSSMNESAKTIKETITELIRQEVISRTINIQSSVQSGIPILLNKQSRDLTQLIEKSCNRQELAQICENTIAIHHADGISPLDPHDFWRCPVGEPLLSNNPNISLLPGPSLLSGSTTISGCVRLPSLSIGDAIYAYSSNLITQGCADIGKSYQVLQLGYISLNSDMYPDLKPVISHTYDINDNRKSCSVIAAGTRGYQLCSLPTVNETTDYSSEGIEDLVFDILDLKGKTKSHRYKNEDITFDHPFSAMYPSVGSGIKIENTLIFLGYGGLTTPLQGDTKCVTNRCANVNQSVCNDALKITWLKKRQVVNVLIRINNYLSDRPKIVVETIPITQNYLGAEGRLLKLGKKIYIYTRSSGWHSHLQIGSLDINNPMTIKWAPHEVLSRPGNQDCNWYNRCPRECISGVYTDAYPLSPDAVNVATTTLYANTSRVNPTIMYSNTSEIINMLRLKNVQLEAAYTTTSCITHFGKGYCFHIVEINQTSLNTLQPMLFKTSIPKICKITS

SEQ ID NO: 753 - protein, 575 aa, Parainfluenza virus type 1; identical to SEQ ID NO: 752

SEQ ID NO: 754 - protein, 575 aa, Parainfluenza virus type 1
MAEKGKTNSSYWSTTRNDNSTVNTYIDTPAGKTHIWLLIATTMHTILSFIIMILCIDLIIKQDTCMKTNIMTVSSMNESAKTIKETITELIRQEVISRTINIQSSVQSGIPILLNKQSRDLTQLIEKSCNRQELAQICENTIAIHHADGISPLDPHDFWRCPVGEPLLSNNPNISLLPGPSLLSGSTTISGCVRLPSLSIGDAIYAYSSNLITQGCADIGKSYQVLQLGYISLNSDMYPDLNPVISHTYDINDNRKSCSVIAAGTRGYQLCSLPTVNETTDYSSEGIEDLVFDILDLKGKTKSHRYKNEDITFDHPFSAMYPSVGSGIKIENTLIFLGYGGLTTPLQGDTKCVTNRCANVNQSVCNDALKITWLKKRQVVNVLIRINNYLSDRPKIVVETIPITQNYLGAEGRLLKLGKKIYIYTRSSGWHSHLQIGSLDINNPMTIKWAPHEVLSRPGNQDCNWYNRCPRECISGVYTDAYPLSPDAVNVATTTLYANTSRVNPTIMYSNTSEIINMLRLKNVQLEAAYTTTSCITHFGKGYCFHIVEINQTSLNTLQPMLFKTSIPKICKITS

SEQ ID NO: 755 - protein, 575 aa, Parainfluenza virus type 1
MAEKGKTNSSYWSTTRNDNSTVNTHINTPAGRTHIWLLIATTMHTVLSFIIMILCIDLIIKQDTCMKTNIMTVSSMNESAKIIKETITELIRQEVISRTINIQSSVQSGIPILLNKQSRDLTQLIEKSCNRQELAQICENTIAIHHADGISPLDPHDFWRCPVGEPLLSNNPNISLLPGPSLLSGSTTISGCVRLPSLSIGDAIYAYSSNLITQGCADIGKSYQVLQLGYISLNSDMYPDLNPVISHTYDINDNRKSCSVIAAGTRGYQLCSLPTVNETTDYSSEGIEDLVFDILDLKGKTKSHRYKNEDITFDHPFSAMYPSVGSGIKIENTLIFLGYGGLTTPLQGDTKCVINRCTNVNQSVCNDALKITWLKKRQVVNVLIRINNYLSDRPKIVVETIPITQNYLGAEGRLLKLGKKIYIYTRSSGWHSNLQIGSLDINNPMTIKWAPHEVLSRPGNQDCNWYNRCPRECISGVYTDAYPLSPDAVNVATTTLYANTSRVNPTIMYSNTSEIINMLRLKNVQLEAAYTTTSCITHFGKGYCFHIVEINQASLNTLQPMLFKTSIPKICKITS

SEQ ID NO: 756 - protein, 547 aa, Synthetic construct
MAEKGKTISSYWSTTRNDNSTVNTHINTPAGRTYIWLLIATTMHAALSLIIMILCIETITELIRQEVISRTINIQSSVQSGIPILLNKQSRDLTQLIEKSCNKQELAQICENTIAIHHADGITPLDPHDFWRCPVGEPLLSNNPNISLLPGPSLLSGSTTISGCVRLPSLSIGDAIYAYSSNLITQGCADIGKSYQVLQLGYISLNSDMYPDLNPVISHTYDINDNRKSCSVIAAGTRGYQLCSLPTVNETTDYSSEGIEDLVFDILDLKGKTKSHRYKNEDITFDHPFSAMYPSVGSGIKIENTLVFLGYGGLTTPLQGNTKCVIKSCPNVNQSVCNDALKITWLKKRQVVNVLIHINNYLSDRPKIVVETIPITQNYLGAEGRLLKLGKKIYIYTRSSGWHSNLQIGSIDINKPMTINWAPHKVLSRPGNPDCNWFNKCPRECISGVYTDAYPLSPDAFNVATTTLYANTSRVNPTIMYSSTSKIINMLRLKNGQLEAAYTTTSCITHFGKGYCFHIVEINQTSLNTLQPMLFKTSIPKICKVTS

SEQ ID NO: 757 - protein, 502 aa, Synthetic construct
MAEKGKTISSYWSTTRNDNSTVNTHINTPAGRTYIWLLIATTMHAALSLIIMILCINKQELAQICENTIAIHHADGITPLDPHDFWRCPVGEPLLSNNPNISLLPGPSLLSGSTTISGCVRLPSLSIGDAIYAYSSNLITQGCADIGKSYQVLQLGYISLNSDMYPDLNPVISHTYDINDNRKSCSVIAAGTRGYQLCSLPTVNETTDYSSEGIEDLVFDILDLKGKTKSHRYKNEDITFDHPFSAMYPSVGSGIKIENTLVFLGYGGLTTPLQGNTKCVIKSCPNVNQSVCNDALKITWLKKRQVVNVLIHINNYLSDRPKIVVETIPITQNYLGAEGRLLKLGKKIYIYTRSSGWHSNLQIGSIDINKPMTINWAPHKVLSRPGNPDCNWFNKCPRECISGVYTDAYPLSPDAFNVATTTLYANTSRVNPTIMYSSTSKIINMLRLKNGQLEAAYTTTSCITHFGKGYCFHIVEINQTSLNTLQPMLFKTSIPKICKVTS

SEQ ID NO: 758 - DNA, 1731 bp, Parainfluenza virus type 1
  source 1..1731: mol_type = other DNA
atggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgacctcatcatcaagcaggatacctgcatgaagaccaacatcatgaccgtgtccagcgtgaacgagagcgccaagacaatcaaagagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatga

SEQ ID NO: 759 - DNA, 1647 bp, Synthetic construct
  source 1..1647: mol_type = other DNA
atggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatga

SEQ ID NO: 760 - DNA, 1512 bp, Synthetic construct
  source 1..1512: mol_type = other DNA
atggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatga

SEQ ID NO: 761 - RNA, 1731 nt, Parainfluenza virus type 1
  source 1..1731: mol_type = mRNA
atggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgacctcatcatcaagcaggatacctgcatgaagaccaacatcatgaccgtgtccagcgtgaacgagagcgccaagacaatcaaagagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatga

SEQ ID NO: 762 - RNA, 1647 nt, Synthetic construct
  source 1..1647: mol_type = other RNA
atggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatga

SEQ ID NO: 763 - RNA, 1512 nt, Synthetic construct
  source 1..1512: mol_type = other RNA
atggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatga

SEQ ID NO: 764 - RNA, 1623 nt, Human Metapneumovirus
  source 1..1623: mol_type = mRNA
atgagctggaaggtggtcatcatcttcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgtgccgatggacccagcctgatcaagaccgagctggacctgacaaagagcgccctgcgggaactgaaaaccgtgtctgcagatcagctggccagagaggaacagatcgagaaccccagacagagcagattcgtgctgggagctatcgccctgggagttgctacagctgctgctgtgacagctggcgtggccattgccaagaccatccggctggaaagcgaagtgaccgccatcaagaacgccctgaaaaagaccaacgaggccgtgtctaccctcggcaatggcgttagagtgctggccacagccgtccgcgagctgaaggatttcgtgtccaagaacctgaccagggccatcaacaagaacaagtgcgacatcgacgacctgaagatggccgtgtccttcagccagttcaaccggcggttcctgaatgtcgtgcggcagttctctgacaacgccggcatcacaccagccatcagcctggatctgatgaccgatgccgaactggctagagccgtgtccaacatgcctacatctgccggccagatcaagctgatgctggaaaacagagccatggtccgacggaaaggcttcggcatcctgatcggcgtgtacggcagcagcgtgatctacatggtgcagctgcctatcttcggcgtgatcgacaccccttgctggatcgtgaaagccgctcctagctgcagcgagaagaagggcaattacgcctgcctgctgagagaggaccaaggctggtactgtcagaatgccggcagcaccgtgtactaccccaacgagaaggactgcgagacaagaggcgaccacgtgttctgtgataccgccgctggaatcaacgtggccgagcagagcaaagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaacagagtgggcatcatcaagcagctgaacaagggctgctcctacatcaccaaccaggacgccgataccgtgaccatcgacaataccgtgtatcagctgagcaaggtggaaggcgaacagcacgtgatcaagggcagacctgtgtccagcagcttcgaccccgtgaagttccccgaggaccagttcaatgtggccctggaccaggtgttcgagaacatcgagaactctcaggctctggtggaccagtccaaccggattctgtctagcgccgagaagggaaacaccggcttcatcatcgtgatcatcctgattgccgtgctgggctccagcatgatcctggtgtccatctttatcatcatcaaaaagacgaagaagcccacaggcgcccctccagaactgtctggcgtgaccaacaatggcttcatccctcacagctgatga

SEQ ID NO: 765 - RNA, 1623 nt, Human Metapneumovirus
  source 1..1623: mol_type = mRNA
atgagctggaaagtcatgatcatcatcagcctgctgatcacccctcagcacggcctgaaagagagctacctggaagaaagctgcagcaccatcaccgagggctacctgagcgtgctgagaaccggctggtacaccaacgtgttcaccctggaagtgggcgacgtggaaaacctgacctgcacagatggccccagcctgatcaagaccgagctggatctgacaaagagcgccctgcgcgagctgaaaaccgtgtctgcagatcagctggccagagaggaacagatcgagaaccccagacagagcagattcgtgctgggagctatcgccctgggagttgctacagctgctgctgtgacagccggaatcgccattgccaagaccatccggctggaaagcgaagtgaacgccatcaagggcgcactgaaaaccaccaacgaggccgtgtctaccctcggcaacggtgttagagtgctggccacagccgtgcgggaactgaaagaattcgtgtccaagaacctgaccagcgccatcaacaagaacaagtgcgacattgccgacctgaagatggccgtgtccttcagccagttcaaccggcggttcctgaatgtcgtgcggcagttctctgacaacgccggcatcacaccagccattagcctggacctgatgaacgacgccgaactggctagagccgtgtcttacatgcctacctctgccggccagatcaagctgatgctggaaaacagagccatggtccgacggaaaggcttcggcatcctgatcggcgtgtacggcagcagcgtgatctacatggtgcagctgcctatcttcggcgtgatcaacaccccttgctggatcatcaaggccgctcctagctgcagcgagaaggacggcaattacgcctgcctgctgagagaggaccaaggctggtactgcaagaatgccggcagcaccgtgtactaccccaacgagaaggattgcgagacacggggcgatcacgtgttctgtgatacagccgccggaatcaacgtggccgagcagagcagagagtgcaacatcaacatcagcaccacaaactacccctgcaaggtgtccaccggcagacaccctatcagcatggtggctctgtctccactgggagccctggtggcttgttataagggcgtgtcctgtagcatcggcagcaatcaagtgggcatcatcaagcagctgcccaagggctgctcctacatcaccaatcaggacgccgacaccgtgaccatcgacaataccgtgtatcagctgagcaaggtggaaggcgaacagcacgtgatcaagggcagacctgtgtccaacagcttcgaccccatcagattccccgaggaccagttcaatgtggccctggaccaggtgttcgagagcatcgagaatagccaggctctggtggaccagtccaacaagatcctgaactccgccgagaagggcaacaccggcttcatcatcgtgatcatcctgattgccgtgctgggcctgaccatgatcagcgtgtccatcatcattatcatcaagaaaacgcggaagcccgccggcgcccctccagaacttaatggcgtgaccaacggcggcttcattccccactcttgatga

SEQ ID NO: 766 - RNA, 1623 nt, Parainfluenza virus type 3
  source 1..1623: mol_type = mRNA
atgctgatctccatcctgctgatcatcaccacaatgatcatggccagccactgccagatcgacatcaccaagctgcagcacgtgggcgtgctggtcaatagccctaagggcatgaagatcagccagaacttcgagacacgctacctgatcctgtctctgatccccaagatcgaggacagcaacagctgcggcgaccagcagatcaagcagtacaagcggctgctggacagactgatcatccctctgtacgacggcctgcggctgcagaaagatgtgatcgtgaccaatcaagagagcaacgagaacacagaccccagaaccgagagattcttcggcggcgtgatcggcacaatcgccctgggagttgctacaagcgcccagattacagccgccgtggctctggtggaagccaagcaggccagaagcgacatcgagaagctgaaagaggccatccgggacaccaacaaggccgtgcagtctgtgcaaagcagcgtgggcaatctgatcgtggccattaagagcgtgcaggactacgtgaacaaagagatcgtcccctctatcgccagactgggatgtgaagctgccggactgcagctgggaattgccctgacacagcactacagcgagctgaccaacatcttcggcgacaacatcggcagcctgcaagagaagggcattaagctgcagggaatcgccagcctgtaccgcaccaacatcaccgagatcttcaccaccagcaccgtggataagtacgacatctacgacctgctgttcaccgagagcatcaaagtgcgcgtgatcgacgtggacctgaacgactacagcatcaccctgcaagtgcggctgcctctgctgaccagactgctgaacacccagatctacaaggtggacagcatctcctacaacatccagaaccgcgagtggtacatcccactgcctagccacattatgaccaagggcgcctttctcggcggagccgatgtgaaagagtgcatcgaggccttcagcagctacatctgccctagcgatcctggcttcgtgctgaaccacgagatggaaagctgcctgagcggcaacatctctcagtgccctagaaccaccgtgacctccgacatcgtgcccagatacgcctttgtgaatggcggcgtggtggccaactgcatcaccaccacctgtacctgcaacggcatcggcaaccggatcaaccagcctccagatcagggcgtgaagattatcacccacaaagagtgtaacaccatcggcatcaacggcatgctgttcaataccaacaaagagggcaccctggccttctacacccctgacgatatcaccctgaacaacagcgtggccctggatcctatcgacatctccatcgagctgaacaaggccaagagcgacctggaagaaagcaaagagtggatccggcggagcaaccagaagctggatagcatcggaagctggcaccagagcagcaccaccatcatcgtgatcctgattatgatgattatcctgttcatcatcaacattaccatcatcacgatcgccatcaagtactaccggatccagaaacggaaccgcgtggaccagaatgacaagccctacgtgctgacaaacaagtgatga

SEQ ID NO: 767 - RNA, 1671 nt, Parainfluenza virus type 1
  source 1..1671: mol_type = mRNA
atgcagagcagcgagatcctgctgctggtgtacagctctctgctgctgagcagctccctgtgtcagatccccgtggacaagctgagcaacgtgggcgtgatcatcaacgagggcaagctgctgaagatcgccggcagctacgagagccggtacatcgtgctgtctctggtgcccagcatcgacctgcaggatggctgtggcaccacacagatcatccagtacaagaacctgctgaaccggctgctgatccctctgaaggatgccctggatctgcaagagagcctgatcaccatcaccaacgacaccaccgtgaccaacgataaccctcagaccagattcttcggcgccgtgatcggaacaatcgccctgggagttgctaccgccgctcagatcacagccggaattgctctggccgaagccagagaggccagaaaggatatcgccctgatcaaggacagcatcgtgaaaacccacaacagcgtggaattcatccagcgcggcatcggcgagcagatcattgccctgaaaaccctgcaggacttcgtgaacgacgagatcagacccgccatcggagagctgagatgcgagacaacagccctgaagctgggcatcaagctgacccagcactacagcgaactggccaccgcctttagcagcaacctgggcacaatcggcgagaagtccctgacactgcaggccctgagcagcctgtacagcgccaatatcacagagatcctgtccaccatcaagaaggacaagagcgacatctacgacatcatctacaccgagcaagtgaagggcaccgtgatcgacgtggacctggaaaagtacatggtcaccctgctggtcaagatccccatcctgtctgagatccctggcgtgctgatctacagagccagcagcatcagctacaacatcgagggcgaagaatggcacgtggcaatccccaactacatcatcaacaaggccagcagcctcggcggagccgatgtgacaaattgcatcgagagcaagctggcctacatctgccccagagatcccacacagctgatccccgacaaccagcagaagtgcatcctgggcgacgtgtccaagtgtcctgtgaccaaagtgatcaacaacctggtgcctaagttcgccttcatcaacggcggcgtggtggccaactgtatcgccagcacatgcacatgcggcaccaacagaatccccgtgaaccaggacagatccaagggcgtgaccttcctgacctacaccaactgtggcctgatcggcatcaatggcatcgagctgtacgccaacaagcggggcagagataccacctggggcaaccagatcatcaaagtgggccctgccgtgtccatccggcctgtggatatctctctgaacctggccagcgccaccaactttctggaagaaagcaagaccgaactcatgaaggccagagccatcatctctgccgttggcggctggcacaacaaagagagcacccagatcattatcatcatcatcgtctgcatcctgatcatcattatctgcggcatcctgtactacctgtacagagtgcggagactgctcatcatgatcaacagcaccaacaatagccccatcaacgcctacacactggaaagccggatgaagaacccctacatgggcaaccacagcaactgatga

SEQ ID NO: 768 - DNA, 1995 bp, Synthetic construct
  source 1..1995: mol_type = other DNA
aggaggactgggcgaacctgcatagtgatcataaggtcatcatagccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgacctcatcatcaagcaggatacctgcatgaagaccaacatcatgaccgtgtccagcgtgaacgagagcgccaagacaatcaaagagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 769 - DNA, 1911 bp, Synthetic construct
  source 1..1911: mol_type = other DNA
aggaggactgggcgaacctgcatagtgatcataaggtcatcatagccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 770 - DNA, 1776 bp, Synthetic construct
  source 1..1776: mol_type = other DNA
aggaggactgggcgaacctgcatagtgatcataaggtcatcatagccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 771 - DNA, 1998 bp, Synthetic construct
  source 1..1998: mol_type = other DNA
aggtgtcagagtttaacttgaagactatttctagggataatatccctgccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgacctcatcatcaagcaggatacctgcatgaagaccaacatcatgaccgtgtccagcgtgaacgagagcgccaagacaatcaaagagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 772 - DNA, 1914 bp, Synthetic construct
  source 1..1914: mol_type = other DNA
aggtgtcagagtttaacttgaagactatttctagggataatatccctgccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 773 - DNA, 1779 bp, Synthetic construct
  source 1..1779: mol_type = other DNA
aggtgtcagagtttaacttgaagactatttctagggataatatccctgccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 774 - RNA, 1995 nt, Synthetic construct
  source 1..1995: mol_type = other RNA
aggaggactgggcgaacctgcatagtgatcataaggtcatcatagccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgacctcatcatcaagcaggatacctgcatgaagaccaacatcatgaccgtgtccagcgtgaacgagagcgccaagacaatcaaagagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 775 - RNA, 1911 nt, Synthetic construct
  source 1..1911: mol_type = other RNA
aggaggactgggcgaacctgcatagtgatcataaggtcatcatagccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 776 - RNA, 1776 nt, Synthetic construct
  source 1..1776: mol_type = other RNA
aggaggactgggcgaacctgcatagtgatcataaggtcatcatagccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 777 - RNA, 1998 nt, Synthetic construct
  source 1..1998: mol_type = other RNA
aggtgtcagagtttaacttgaagactatttctagggataatatccctgccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgacctcatcatcaagcaggatacctgcatgaagaccaacatcatgaccgtgtccagcgtgaacgagagcgccaagacaatcaaagagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 778 - RNA, 1914 nt, Synthetic construct
  source 1..1914: mol_type = other RNA
aggtgtcagagtttaacttgaagactatttctagggataatatccctgccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcgagacaatcaccgagctgatccggcaagaagtgatcagccggaccatcaacatccagtcctctgtgcagagcggcatccccatcctgctgaacaagcagagcagagatctgacccagctgatcgagaagtcctgcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 779 - RNA, 1779 nt, Synthetic construct
  source 1..1779: mol_type = other RNA
aggtgtcagagtttaacttgaagactatttctagggataatatccctgccaccatggccgagaagggcaagaccatcagcagctactggtccaccaccagaaacgacaacagcaccgtgaacacccacatcaacaccccagccggcagaacctacatctggctgctgatcgccaccaccatgcacgctgccctgagcctgatcatcatgatcctgtgcatcaacaagcaagagctggcccagatctgcgagaacacaatcgccattcaccacgccgacggcatcacccctctggaccctcacgatttttggagatgccctgtgggcgagcccctgctgagcaacaaccccaatatcagcctgctgcctggaccttctctgctgagcggcagcacaacaatctctggctgcgtcagactgcccagcctgtctatcggagatgccatctacgcctacagcagcaacctgatcacacagggctgcgccgacatcggcaagagctatcaggttctgcagctgggctacatcagcctgaacagcgacatgtaccccgatctgaaccccgtgatctcccacacctacgacatcaacgacaaccgcaagtcctgctccgtgatcgccgctggcacaagaggataccagctgtgtagcctgcctaccgtgaacgaaaccaccgactacagctccgagggcatcgaggacctggtgttcgacatcctggacctgaagggaaagacaaagagccaccggtacaagaacgaggacatcaccttcgatcaccccttcagcgccatgtatcctagcgtcggcagcggcatcaagatcgagaacaccctggtgtttctcggctacggcggcctgacaacacctctgcagggcaacaccaaatgcgtgatcaagagctgccctaacgtgaaccagtccgtgtgcaacgacgccctgaagatcacctggctgaagaaacggcaggtcgtgaacgtgctgatccacattaacaactacctgagcgacaggcccaagatcgtggtggaaacaatccccatcacacagaactacctgggcgccgaaggcagactgctgaagctgggcaagaagatctacatctacaccagaagcagcggctggcactccaacctgcagatcggcagcatcgatatcaacaagcccatgaccatcaattgggcccctcacaaggtgctgagcagacccggaaatcccgactgcaactggttcaacaagtgccccagagaatgcatcagcggcgtgtacaccgacgcctatcctctgagccccgacgcctttaacgtggccaccacaacactgtacgccaacaccagcagagtgaaccccaccatcatgtacagctccaccagcaagatcatcaacatgctgagactgaagaacggccagctggaagccgcctacaccaccacaagctgcatcacccacttcggcaagggctactgcttccacatcgtggaaatcaaccagacctctctgaacacactgcagcccatgctgttcaagaccagcattcccaagatctgcaaagtgaccagctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 780 - RNA, 1728 nt, Respiratory Syncytial virus
  source 1..1728: mol_type = mRNA
atggaactgcccatcctgaaaacaaacgccatcaccaccatcctggccgccgtgacactgtgttttgccagcagccagaacatcaccgaggaattctaccagagcacctgtagcgccgtgtccaagggctatctgagcgcccttagaaccggctggtacaccagcgtgatcaccatcgagctgagcaacatcaaagaaaacaagtgcaacggcaccgacgccaaagtgaagctgatcaagcaagagctggacaagtacaagaacgccgtgaccgaactgcagctgctgatgcagtctacccctgccgccaatagcagagccagacgggaactgcctagattcatgaactacaccctgaacaacaccaagaacaccaacgtgaccctgagcaagaagcggaagcggagattcctgggctttctgctcggagtgggaagcgccattgcctctggaatcgccgtgtctaaagtgctgcacctggaaggcgaagtgaacaagatcaagagcgccctgctgagcaccaacaaggccgtggtgtctctgagcaatggcgtgtccgtgctgaccagcaaggtgctggacctgaagaactacatcgacaaacagctgctgcccatcgtcaacaagcagagctgcagcatcagcaacatcgagacagtgatcgagttccagcagaagaacaaccggctgctggaaatcacccgcgagttctctgtgaatgccggcgtgaccacacctgtgtccacctacatgctgaccaacagcgagctgctgtccctgatcaacgacatgcccatcaccaacgaccagaaaaagctgatgagcagcaacgtgcagatcgtgcggcagcagagctactccatcatgagcattatcaaagaagaggtgctggcctacgtggtgcagctgcctctgtatggcgtgatcgataccccttgctggaagctgcacacaagccctctgtgcaccaccaacaccaaagagggctccaacatctgcctgaccagaaccgacagaggctggtactgcgataatgccggcagcgtctcattcttcccacaagccgagacatgcaaggtgcagagcaaccgggtgttctgcgacaccatgaacagcctgacactgccctccgaagtgaatctgtgcaatatcgacatcttcaacccgaagtacgactgcaagatcatgacctccaagaccgacgtgtccagcagtgtgatcacctctctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagcaagaaggcaagagcctgtacgtgaagggcgagcccatcatcaacttctacgaccctctggtgttccccagcgacgagttcgatgccagcatctcccaagtgaacgagaagatcaaccagagcctggccttcatcagaaagtccgatgagctgctgcacaacgtgaacgccggcaagtccaccaccaatatcatgatcacgaccatcatcatcgtgattatcgtgatcctgctggctctgatcgccgtgggcctgctgctgtattgcaaggccagatctaccccagtgactctgtccaaggatcagctgagcggcatcaacaatatcgccttctccaactgatga

SEQ ID NO: 781 - RNA, 1728 nt, Respiratory Syncytial Virus
  source 1..1728: mol_type = mRNA
atggaactgctgatccacagaagcagcgccatctttctgaccctggccatcaacgccctgtacctgaccagcagccagaacatcaccgaggaattctaccagagcacctgtagcgccgtgtccagaggctactttagcgccctgagaaccggctggtacaccagcgtgatcaccatcgagctgagcaacatcaaagaaacgaagtgcaacggcaccgacaccaaagtgaagctgatcaagcaagagctggacaagtacaagaacgccgtgaccgaactgcagctgctgatgcagaatacccctgccgccaacaaccgggccagaagagaagcccctcagtacatgaactacaccatcaacaccaccaagaacctgaacgtgtccatcagcaagaagcggaagcggcggttcctgggctttctgcttggagtgggaagcgccattgccagcggaatcgccgtgtctaaagtgctgcacctggaaggcgaagtgaacaagatcaagaatgccctgctgagcaccaacaaggccgtggtgtctctgagcaatggcgtgtccgtgctgacctccaaggtgctggacctgaagaactacatcaacaaccagctgctgcccatcgtgaaccagcagagctgccggatcagcaacatcgagacagtgatcgagttccagcagaagaacagcaggctgctggaaatcacccgcgagttctctgtgaatgccggcgtgacaacacccctgagcacctacatgctgaccaacagcgagctgctgtccctgatcaacgacatgcccatcaccaacgaccagaaaaagctgatgagcagcaacgtgcagatcgtgcggcagcagtcctacagcatcatgagcattatcaaagaagaggtgctggcctacgtggtgcagctgcctatctacggcgtgatcgataccccttgctggaagctgcacacaagccctctgtgcaccaccaatatcaaagagggctccaacatctgcctgaccagaaccgacagaggctggtactgcgataatgccggcagcgtctcattcttcccacaagccgatacctgcaaggtgcagagcaaccgggtgttctgcgacaccatgaacagcctgacactgccctctgaggtgtccctgtgcaacaccgacatcttcaactctaagtacgactgcaagatcatgaccagcaagaccgatatcagctcctccgtgatcacaagcctgggcgccatcgtgtcctgttacggcaagaccaagtgcaccgccagcaacaagaaccggggcatcatcaagaccttcagcaacggctgcgactacgtgtccaacaaaggcgtggacaccgtgtctgtgggcaacaccctgtactacgtgaacaagctggaagggaagaacctgtatgtgaagggcgagcccatcatcaactactacgaccctctggtgttccccagcgacgagttcgatgccagcatcagccaagtgaacgagaagatcaaccagagcctggccttcatcaggcggagcgacgaactgctgcacaatgtgaacaccggcaagtccaccacaaacatcatgatcaccgccatcatcatcgtgatcattgtggtgctgctgagcctgatcgccatcggcctgctgctgtattgcaaggccaagaacaccccagtgacactgagcaaggatcagctgagcggcatcaacaatatcgccttctccaagtgatga

(19) United States
(12) Patent Application Publication
(10) Pub. No.: US 2026/0021175 A1
(43) Pub. Date: Jan. 22, 2026

(54) IMMUNOGENIC COMPOSITIONS AND USES THEREOF

(71) Applicant: Pfizer Inc., New York, NY (US)

(72) Inventors: Bridget Yih Jiin Huang, New York, NY (US); Weiqiang Li, Brooklyn, NY (US); Kena Anne Swanson, Pearl River, NY (US)

(21) Appl. No.: 19/270,518

(22) Filed: Jul. 16, 2025

Related U.S. Application Data
(60) Provisional application No. 63/672,697, filed on Jul. 17, 2024.
(60) Provisional application No. 63/762,997, filed on Feb. 25, 2025.
(60) Provisional application No. 63/821,157, filed on Jun. 10, 2025.
(60) Provisional application No. 63/841,690, filed on Jul. 10, 2025.

(51) Int. Cl. A61K 39/155; A61K 9/1272; A61K 9/51; A61K 39/00; A61K 47/12; A61P 31/14; C07K 14/005; C12N 7/00; C12N 9/24

(52) U.S. Cl. CPC A61K 39/155; A61K 9/1272; A61K 9/5123; A61K 47/12; A61P 31/14; C07K 14/005; C12N 7/00; C12N 9/2402; A61K 2039/53; A61K 2039/545; A61K 2039/55555; A61K 2039/575; A61K 2039/70; C07K 2319/00; C12N 2760/18322; C12N 2760/18334; C12N 2760/18522; C12N 2760/18534; C12N 2760/18622; C12N 2760/18634; C12Y 302/01018

(57) ABSTRACT

The present disclosure relates to RSV F, hMPV F, PIV3 F, PIV3 HN, PIV1 F and PIV1 HN protein mutants, nucleic acids or vectors encoding them, compositions comprising a RSV F, hMPV F, PIV3 F, PIV3 HN, PIV1 F and/or PIV1 HN nucleic acid or combinations thereof, and uses thereof.

### CROSS REFERENCE TO RELATED APPLICATIONS

This application claims the benefit of and U.S. Provisional Application No. 63/841,690, filed Jul. 10, 2025, U.S. Provisional Application No. 63/821,157, filed Jun. 10, 2025, U.S. Provisional Application No. 63/762,997, filed Feb. 25, 2025 and U.S. Provisional Application No. 63/672,697, filed Jul. 17, 2024. The entire content of each of the foregoing applications is hereby incorporated herein by reference.

### REFERENCE TO SEQUENCE LISTING

This application is being filed electronically via EFS-Web and includes an electronically submitted sequence listing in .xml format. The .xml file contains a sequence listing entitled “PC073150A Sequence Listing.xml” created on Jul. 11, 2025 and having a size of 948 KB. The sequence listing contained in this .xml file is part of the specification and is hereby incorporated herein by reference in its entirety.

### BACKGROUND OF THE INVENTION

Human paramyxoviruses and pneumoviruses are widespread pathogens, cause considerable disease burden, and include measles virus (MeV), mumps virus (MuV), respiratory syncytial virus (RSV), metapneumovirus (MPV), and parainfluenza virus types 1˜4 (PIV1-4).

Respiratory syncytial virus (RSV) is a respiratory virus that infects the lungs and breathing passages. RSV is the leading cause of serious viral lower respiratory tract illness in infants worldwide and an important cause of respiratory illness in the elderly. Two RSV protein subunit vaccines were approved in 2023, ABRYSVO (Pfizer) and AREXVY (GSK), and one mRNA vaccine was approved in 2024, mRESVIA (Moderna), for RSV disease. RSV is a member of the Pneumoviridae family. Its genome consists of a single-stranded, negative-sense RNA molecule that encodes 11 proteins, including nine structural proteins (three glycoproteins and six internal proteins) and two non-structural proteins. The structural proteins include three transmembrane surface glycoproteins: the attachment protein G, fusion protein F, and the small hydrophobic SH protein. There are two subtypes of RSV, A and B. They differ primarily in the G glycoprotein, while the sequence of the F glycoprotein is more conserved between the two subtypes.

The mature F glycoprotein has three general domains: ectodomain (ED), transmembrane domain (TM), and a cytoplasmic tail (CT). CT contains a single palmitoylated cysteine residue.

The F glycoprotein of human RSV is initially translated from the mRNA as a single 574-amino acid polypeptide precursor (referred to “F0” or “F0 precursor”), which contains a signal peptide sequence (amino acids 1-25) at the N-terminus. Upon translation the signal peptide is removed by a signal peptidase in the endoplasmic reticulum. The remaining portion of the F0 precursor (e.g., residues 26-574) may be further cleaved at two polybasic sites (a.a. 109/110 and 136/137) by cellular proteases (in particular furin), removing a 27-amino acid intervening sequence designated pep27 (amino acids 110-136) and generating two linked fragments designated F1 (C-terminal portion; amino acids 137-574) and F2 (N-terminal portion; amino acids 26-109). F1 contains a hydrophobic fusion peptide at its N-terminus and two heptad-repeat regions (HRA and HRB). HRA is near the fusion peptide, and HRB is near the TM domain. The F1 and F2 fragments are linked together through two disulfide bonds. Either the uncleaved F0 protein without the peptide sequence or a F1-F2 heterodimer can form a RSV F protomer. Three such protomers assemble to form the final RSV F protein complex, which is a homotrimer of the three protomers.

The F proteins of subtypes A and B are about 90 percent identical in amino acid sequence. An example sequence of the F0 precursor polypeptide for the A subtype is provided in SEQ ID NO: 1 (A2 strain; GenBank GI: 138251; Swiss Prot P03420), and for the B subtype is provided in SEQ ID NO: 2 (18537 strain; GenBank GI: 138250; Swiss Prot P13843). SEQ ID NO: 1 and SEQ ID NO: 2 are both 574 amino acid sequences. The signal peptide sequence for SEQ ID NO: 1 and SEQ ID NO: 2 has also been reported as amino acids 1-25 (GenBank and UniProt). In both sequences the TM domain is from approximately amino acids 530 to 550 but has alternatively been reported as 525-548. The cytoplasmic tail begins at either amino acid 548 or 550 and ends at amino acid 574, with the palmitoylated cysteine residue located at amino acid 550.

RSV F protein is a primary antigen explored for RSV vaccines. The RSV F protein trimer mediates fusion between the virion membrane and the host cellular membrane and also promotes the formation of syncytia. In the virion prior to fusion with the membrane of the host cell, the largest population of F molecules forms a lollipop-shaped structure, with the TM domain anchored in the viral envelope [Dormitzer, P. R., Grandi, G., Rappuoli, R., Nature Reviews Microbiol, 10, 807, 2012.]. This conformation is referred to as the pre-fusion conformation. Pre-fusion RSV F is recognized by monoclonal antibodies (mAbs) D25, AM22, and MPE8, without discrimination between oligomeric states. Pre-fusion F trimers are specifically recognized by mAb AM14 [Gilman M S, Moin S M, Mas V et al., PLOS Pathogens, 11 (7), 2015]. During RSV entry into cells, the F protein rearranges from the pre-fusion state (which may be referred to herein as “pre-F”), through an intermediate extended structure, to a post-fusion state (“post-F”). During this rearrangement, the C-terminal coiled-coil of the pre-fusion molecule dissociates into its three constituent strands, which then wrap around the globular head and join three additional helices to form the post-fusion six helix bundle. If a pre-fusion RSV F trimer is subjected to increasingly harsh chemical or physical conditions, such as elevated temperature, it undergoes structural changes. Initially, there is loss of trimeric structure (at least locally within the molecule), and then rearrangement to the post-fusion form, and then denaturation of the domains.

To prevent viral entry, F-specific neutralizing antibodies presumably must bind the pre-fusion conformation of F on the virion, or potentially the extended intermediate, before the viral envelope fuses with a cellular membrane. Thus, the pre-fusion form of the F protein is considered the preferred conformation as the desired vaccine antigen [Ngwuta, J. O., Chen, M., Modjarrad, K., Joyce, M. G., Kanekiyo, M., Kumar, A., Yassine, H. M., Moin, S. M., Killikelly, A. M., Chuang, G. Y., Druz, A., Georgiev, I. S., Rundlet, E. J., Sastry, M., Stewart-Jones, G. B., Yang. Y., Zhang, B., Nason, M. C., Capella, C., Peeples, M., Ledgerwood, J. E., Mclellan, J. S., Kwong, P. D., Graham, B. S., Science Translat. Med., 14, 7, 309 (2015)]. Upon extraction from a membrane with surfactants such as Triton X-100, Triton X-114, NP-40, Brij-35, Brij-58, Tween 20, Tween 80, Octyl glucoside, Octyl thioglucoside, SDS, CHAPS, CHAPSO, or expression as an ectodomain, physical or chemical stress, or storage, the F glycoprotein readily converts to the post-fusion form [Mclellan J S, Chen M, Leung S et al. Structure of RSV fusion glycoprotein trimer bound to a pre-fusion-specific neutralizing antibody. Science 340, 1113-1117 (2013); Chaiwatpongsakorn, S., Epand, R. F., Collins, P. L., Epand R. M., Peeples, M. E., J Virol. 85 (8): 3968-77 (2011); Yunus, A. S., Jackson T. P., Crisafi, K., Burimski, I., Kilgore, N. R., Zoumplis, D., Allaway, G. P., Wild, C. T., Salzwedel, K. Virology. 2010 Jan. 20; 396 (2): 226-37]. Therefore, the preparation of prefusion F as a vaccine antigen has remained a challenge. Since the neutralizing and protective antibodies function by interfering with virus entry, it is postulated that an F antigen that does not elicit pre-fusion specific antibodies is not expected to be as effective as an F antigen that elicits pre-fusion specific antibodies. Therefore, it is considered more desirable to utilize an F protein vaccine that contains a F protein immunogen in the pre-fusion form. Mutants of the RSV F protein have been provided to increase pre-fusion stability (see for example PCT application No WO2017/109629) and are promising vaccine candidates.

RSV vaccines that incorporate F protein antigen have been under development. Clinical studies have shown that some F protein subunit-based vaccine candidates are efficacious, safe and immunogenic (e.g. ABRYSVO or AREXVY). However, improvements in protective efficacy and durability of protection are desirable.

Human metapneumovirus (hMPV) is a respiratory virus that infects the lungs and breathing passages. HMPV is a clinically important respiratory virus that results in substantial disease burden in children and accounts for significant pediatric hospitalization.

There is near ubiquitous infection by the age of five and re-infections continue to be a burden throughout life (van den Hoogen et al., 2001). However, infants (6-12 months), the elderly, and immunocompromised populations are at an increased risk of hospitalization with more severe disease such as pneumonia and bronchiolitis (Deffrasnes et al., 2007). Despite the disease burden that hMPV presents, there are no vaccines or therapeutics that have been approved for prevention or treatment.

hMPV is a member of the Pneumoviridae family, and its genome comprises three surface glycoproteins: the attachment protein G, fusion protein F, and the small hydrophobic SH protein. There are two subtypes of hMPV, A and B. They differ primarily in the G glycoprotein, while the sequence of the F glycoprotein is more conserved between the two subtypes.

The mature F glycoprotein has three general domains: ectodomain (ED), transmembrane domain (TM), and a cytoplasmic tail (CT).

The F glycoprotein of hMPV is initially translated from the mRNA as a single 539-amino acid polypeptide precursor (referred to as “F0” or “F0 precursor”), which contains a signal peptide sequence (amino acids 1-18) at the N-terminus. Upon translation the signal peptide is removed by a signal peptidase in the endoplasmic reticulum.

The remaining portion of the F0 precursor (e.g., residues 18-539) may be further cleaved at position 102/103 by cellular proteases to generate two linked fragments designated F1 (C-terminal portion; amino acids 103-539) and F2 (N-terminal portion; amino acids 19-102). F1 contains a hydrophobic fusion peptide at its N-terminus and two heptad-repeat regions (HRA and HRB). HRA is near the fusion peptide, and HRB is near the TM domain. The F1 and F2 fragments are linked together through two disulfide bonds. Either the uncleaved F0 protein without the signal peptide sequence or a F1-F2 heterodimer can form a hMPV F protomer. Three such protomers assemble to form the final hMPV F protein complex, which is a homotrimer of the three protomers.

The F proteins of subtypes A and B are well conserved and an example sequence of the F0 precursor polypeptide for the A subtype is provided in SEQ ID NO: 1 (A2b strain (TN/95/3-54) GenBank GI: ACJ53569.1)), and for the B subtype is provided in SEQ ID NO: 4 (consensus sequence). SEQ ID NO:639 and SEQ ID NO:640 are both 539 amino acid sequences. The signal peptide sequence for SEQ ID NO:639 and SEQ ID NO:640 consists of amino acids 1-18.

One of the primary antigens explored for hMPV subunit vaccines is the F protein. The hMPV F protein trimer mediates fusion between the virion membrane and the host cellular membrane and also promotes the formation of syncytia. In the virion prior to fusion with the membrane of the host cell, the largest population of F molecules forms a lollipop-shaped structure, with the TM domain anchored in the viral envelope. This conformation is referred to as the prefusion conformation. Prefusion hMPV A F is recognized for example by monoclonal antibodies (mAbs) MPE8, without discrimination between oligomeric states. During hMPV entry into cells, the F protein rearranges from the prefusion state (which may be referred to herein as “pre-F”), through an intermediate extended structure, to a post-fusion state (“post-F”). During this rearrangement, the C-terminal coiled-coil of the prefusion molecule dissociates into its three constituent strands, which then wrap around the globular head and join three additional helices to form the post-fusion six helix bundle. If a prefusion hMPV F trimer is subjected to increasingly harsh chemical or physical conditions, such as elevated temperature, it undergoes structural changes. Initially, there is loss of trimeric structure (at least locally within the molecule), and then rearrangement to the post-fusion form, and then denaturation of the domains.

To prevent viral entry, F-specific neutralizing antibodies presumably must bind the prefusion conformation of F on the virion, or potentially the extended intermediate, before the viral envelope fuses with a cellular membrane. Thus, the prefusion form of the F protein is considered the preferred conformation as the desired vaccine antigen (Stewart Jones et al, PNAS 2021 Vol. 118 No. 39 and Hsieh et al, Nature Communications volume 13, Article number: 1299 (2022). However, the exact role of hMPV F prefusion form in eliciting immunogenicity is less established in comparison with RSV F. Upon extraction from a membrane with surfactants or expression as an ectodomain, physical or chemical stress, or storage, the F glycoprotein readily converts to the post-fusion form (Más et al, 2016 PLOS Pathog 12 (9): e1005859).

PIV1 and PIV3 (genus Respirovirus) are important pediatric pathogens within the Paramyxoviridae family, with lower incidence or disease severity caused by the paramyxovirus family members PIV2 and PIV4. While effective responses to measles and mumps can be induced by live attenuated viral vaccines, licensed vaccines for PIV1 and PIV3 have not been obtained using the same approach. Entry by these viruses also utilizes the viral fusion (F) glycoprotein, as disclosed above for hMPV.

The preparation of hMPV, PIV1 or PIV3 prefusion F as a vaccine antigen has remained a challenge. Since the neutralizing and protective antibodies function by interfering with virus entry, it is postulated that an F antigen that is unable to elicit pre-fusion specific antibodies is not expected to be as effective as an F antigen that elicits prefusion specific antibodies. Therefore, it is considered more desirable to utilize a vaccine that contains a F protein immunogen stabilized in the prefusion form.

While prefusion F protein is the main target for RSV and hMPV neutralization responses, the receptor binding protein, hemagglutinin-neuraminidase protein (HN), of PIV3 has been demonstrated as a key neutralizing antibody target as well as it is a surface glycoprotein that plays a crucial role in viral infection. The HN protein mediates the attachment to the sialic acid residues on the host cell surface and facilitates the release of the new virions from the cell by cleaving the sialic acid residues (Huberman et al. 1995, Moscona 1997). By forming a complex with the F protein on the virion surface, HN stabilizes the prefusion F protein prior to receptor engagement and induces F protein conformational change for cell membrane fusion upon host cell attachment (Chang and Dutch 2012). Early and recent studies have identified potent neutralizing antibodies targeting HN, further demonstrating the critical role of HN in viral life cycle (Henrickson and Portner 1990, Miller et al. 2024, Suryadevara et al. 2024, van Wyke Coelingh et al. 1985). Additionally, PIV1 and PIV3 HN proteins share 49% sequence homology, which suggests the potential for cross-reactive mAb binding (Miller et al. 2024).

Efforts to date have not yielded licensure of an hMPV, PIV1 or PIV3 vaccine. Therefore, there is a need for immunogens derived from F protein of hMPV, PIV1 and PIV3, and HN protein of PIV1 and/or PIV3 and compositions comprising such immunogens, such as a vaccine.

There is also a need for a respiratory vaccine comprising a combination of RSV, hMPV, and/or PIV3 and/or PIV1 protein antigens in a single vaccine to provide protection against several viruses causing respiratory diseases.

RNA technology, especially mRNA technology, is particularly advantageous as a vaccine or therapeutic platform. For an effective RNA vaccine or therapeutic, it is important to maximize protein expression such that amounts of desired proteins or antigens are generated from minimal amounts of RNAs. However, mRNA-based therapies can suffer from challenges including low manufacturing efficiency, short half-life of administered mRNA in circulation, and low translation efficiency. As such, there is a need for RNA compositions with improved stability and translation efficiency, including methods to improve protein expression by optimizing the sequence and structure of the 5′ untranslated regions of the mRNA and enable high levels of expression.

### SUMMARY

The present invention provides for the unmet need for immunogenic compositions against RSV, hMPV and/or PIV3 and/or PIV1 infection, as provided herein.

Exemplary Embodiments (E) of the invention are set forth below:
E1. An RNA molecule comprising at least one open reading frame encoding a human Metapneumovirus (hMPV) fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.
E2. An RNA molecule comprising at least one open reading frame encoding a parainfluenza virus type 3 (PIV3) fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.
E3. An RNA molecule comprising at least one open reading frame encoding a parainfluenza virus type 3 (PIV3) hemagglutinin-neuraminidase protein (HN) polypeptide.
E4. An RNA molecule comprising at least one open reading frame encoding a parainfluenza virus type 1 (PIV1) fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.
E5. An RNA molecule comprising at least one open reading frame encoding a parainfluenza virus type 1 (PIV1) hemagglutinin-neuraminidase protein (HN) polypeptide.
E6. An immunogenic composition comprising at least one RNA molecule of any one of embodiments 1-5, wherein the RNA molecule is formulated in a lipid nanoparticle (LNP) thereby forming an RNA-LNP. The immunogenic composition of embodiment 106, wherein the RNA molecule is selected from the group consisting of an RNA molecule encoding hMPV F A, hMPV F B, PIV3 F, PIV3 HN, PIV1 F and/or PIV1 HN.
E7. The immunogenic composition of embodiment 6, further comprising an RNA molecule comprising at least one open reading frame encoding a respiratory syncytial virus (RSV) fusion protein F (F) polypeptide and a 5′ untranslated region (RSV 5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.
E8. The immunogenic composition of any one of embodiments E6 or E7, wherein the RNA molecules are formulated in one or more lipid nanoparticles (RNA-LNP).
E9. The immunogenic composition of any one of embodiments E6-E8, wherein the immunogenic composition comprises one or more RNA-LNPs selected from:
   i. an RNA-LNP comprising one RNA molecule,
   ii. an RNA-LNP comprising two or more co-formulated RNA molecules that do not encode the same antigen (pre-mixed), or
   iii. a mixture of two or more RNA-LNPs selected from (i) or (ii) (post-mix).
E10. A mutant of a wild-type parainfluenza virus type 1 (PIV1) HN polypeptide, wherein the mutant comprises at least one amino acid mutation relative to the amino acid sequence of the wild type PIV1 HN polypeptide, wherein the mutation is selected from the group consisting of: i) a deletion of residues at positions 57-84; and ii) a deletion of residues at positions 57-129, wherein the amino acid positions are numbered according to SEQ ID NO: 750.
E11. A mutant of a wild-type parainfluenza virus type 3 (PIV3) HN polypeptide, wherein the mutant comprises at least one amino acid mutation relative to the amino acid sequence of the wild type PIV3 HN polypeptide, wherein the mutation is selected from the group consisting of: i) a deletion of residues at positions 59-88; and ii) a deletion of residues at positions 59-130, wherein the amino acid positions are numbered according to SEQ ID NO: 724.
E12. The immunogenic composition of any one of the above-mentioned embodiments for use as a vaccine.

It is contemplated that any aspect discussed in this specification may be implemented with respect to any method or composition of the disclosure, and vice versa. Furthermore, compositions of the disclosure may be used to achieve methods of the disclosure.

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect. Use of the one or more compositions may be employed based on any of the methods described herein.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

FIG. 1 schematically illustrates the wild-type (WT) RSV F protein (RSV WT) and variant RSV F protein constructs, where “SP” refers to a signal peptide sequence (amino acid residues 1-25 of each construct). The amino acid positions of each portion (e.g. SP, F2, pep27, F1) or mutation for each construct are indicated therein, e.g. SP of each construct spans from amino acid residues 1-25 of each construct. Each RSV protein construct depicted is applicable to both RSV subtype A and subtype B constructs of the WT or variant thereof as shown.

FIG. 2 depicts RSV-F expression by candidate UTR constructs with hDC expression along the X-axis and HEK expression along the Y-axis. Labeled constructs (also denoted by darker gray plot points) were nominated for in vivo testing in mice. WHO benchmark is denoted by the intersection of the lines. Additional benchmarks are indicated by asterisks (*).

FIG. 3A-3B depicts RSV-F neutralizing titers from mice injected with modRNA LNPs expressing RSV-F driven by different UTR combinations after 21 days (21 days post dose 1) (FIG. 3A) and after 35 days (14 days post dose 2) (FIG. 3B). The two WHO benchmark constructs with 5′/3′ UTRs with different polyA tails are denoted as 5UTR_1/3UTR_1 constructs. Female BALB/c mice (10/group) were immunized intramuscularly at day 0 and 21 with RSV-F subgroup A modRNA-LNPs with novel UTR candidates at 0.5 μg dose. On day 21 (3 weeks post dose 1, 3W PD1) and day 35 (2 weeks post dose 2, 2W PD2), serum was collected for RSV neutralizing assay. Neutralization assay results for RSV A on day 21 (FIG. 3A) and day 35 (FIG. 3B) expressed as 50% neutralizing titers. Each symbol represents a titer from an individual animal. Horizontal line represents geometric mean titer (GMT) of the group.

FIG. 4 is a diagram of the organization of the translation machinery. The 5′ cap and part of the 5′ UTR interact with the 43S pre-initiation complex. The 43S complex then scans the 5′ UTR towards the AUG start site, where it assembles with the 60S complex. Following the release of the elongation factors, the ribosome starts translation (adapted from Leppek et al., Nat Rev Mol Cell Biol 2018).

FIG. 5A-5B depict RSV-F neutralizing titers from mice injected with modRNA LNPs expressing RSV-F driven by different UTR combinations after 35 days (2 weeks post dose 2) in an initial (FIG. 5A) and a follow-on (FIG. 5B) experiment. Benchmark constructs with 5′/3′ UTRs with different polyA tails are denoted. Female BALB/c mice (10/group) were immunized intramuscularly at day 0 and 21 with RSV-F subgroup A modRNA-LNPs with novel UTR candidates at 0.2 μg dose. On day 35 (2 weeks post dose 2, 2W PD2), serum was collected for RSV neutralizing assay. Neutralization assay results for RSV A on day 35 expressed as 50% neutralizing titers. Each symbol represents a titer from an individual animal. Horizontal line represents geometric mean titer (GMT) of the group.

FIG. 6A-6C depict 50% neutralizing titers in PD2 mouse sera raised in various hMPV studies: FIG. 6A depicts 50% neutralizing titers in PD2 mouse sera raised against different UTR designs in combination with hMPV F hMPV021 encoded from 0.5 μg LNP-formulated modRNA; FIG. 6B depicts 50% neutralizing titers in PD2 mouse sera raised against different UTR designs in combination with hMPV A F mutants encoded from 0.5 μg LNP-formulated modRNA; and FIG. 6C depicts 50% neutralizing titers in PD2 mouse sera raised against different UTR designs in combination with hMPV B F mutants encoded from 0.5 μg LNP-formulated modRNA. Dotted line represents the limit of detection at 20 in FIG. 6A-6C.

FIG. 7 depicts 50% neutralizing titers in PD2 mouse sera raised against different UTR designs in combination with PIV1047 F encoded from 0.2 μg LNP-formulated modRNA. Dotted line represents the limit of detection at 20.

FIG. 8A-8I depict 50% neutralizing titers in PD2 mouse sera raised in various PIV3 studies: FIG. 8A depicts 50% neutralizing titers in PD2 mouse sera raised against different UTR designs in combination with PIV3 F PIV3008 mutant encoded from 0.2 μg LNP-formulated modRNA; FIG. 8B depicts 50% neutralizing titers in PD2 mouse sera raised against different UTR designs in combination with PIV3 F mutants encoded from 0.2 μg LNP-formulated modRNA; FIG. 8C depicts 50% neutralizing titers in PD2 mouse sera raised against different UTR designs in combination with PIV3 F mutants encoded from 0.05 μg LNP-formulated modRNA; FIG. 8D depicts 50% neutralizing titers in PD2 mouse sera raised against PIV3 HN WT with 0.2 μg LNP-formulated modRNA expressed through either novel UTR designs or WHO/WHO benchmark UTR; FIG. 8E depicts 50% neutralizing titers in PD2 mouse sera raised against different PIV3 HN mutants with 0.4 μg LNP-formulated modRNA expressed through 5UTR_582/hHBB; FIG. 8F depicts 50% neutralizing titers in PD2 mouse sera raised against different PIV3 HN mutants with 0.2 μg LNP-formulated modRNA expressed through 5UTR_582/hHBB; and FIG. 8G depicts 50% neutralizing titers in PD2 mouse sera raised against bivalent (0.2 μg PIV3 F+0.2 μg PIV3 HN) modRNA-LNP, expressed through 5UTR_582/hHBB. Dotted line represents the limit of detection at 20 in FIG. 8A-8G. FIG. 8H shows post-natural infection human sera neutralization activity with or without depletion with PIV3 preF and/or HN proteins. 50% neutralization titers are shown. LOD (limit of detection) is 40. Median % Reduction was calculated using the unabsorbed titer as a reference. FIG. 8I shows representations of the different PIV3 HN designs with truncation in the stalk domain and comparison against the WT. The symbol A denotes the deletion of amino acids at the indicated positions, e.g. 459-88 denotes deletion of amino acids at positions 59-88 of the PIV1 HN polypeptide.

FIG. 9A-9B show the results of an in vivo immunogenicity study for Flu HA/Cali using ESM formulations at 3 weeks post dose 1 (3 wks PD1; FIG. 9A) and 2 weeks post dose 2 (2 wks PD2; FIG. 9B).

FIG. 10A-10B show the results of an in vivo immunogenicity study for RSV preF using ESM formulations at 3 weeks post dose 1 (3 wks PD1; FIG. 10A) and 2 weeks post dose 2 (2 wks PD2; FIG. 10B).

FIG. 11A-11B show the results of an in vivo immunogenicity study for H1N1 A/California using ESM formulations at 3 weeks post dose 1 (3 wks PD1; FIG. 11A) and 2 weeks post dose 2 (2 wks PD2; FIG. 11B).

FIG. 12 shows a protein sequence alignment of PIV1 HN WT strains using CLUSTAL O(1.2.4) multiple sequence alignment tool.

FIG. 13 shows a protein sequence alignment of PIV3 HN WT strains using CLUSTAL O(1.2.4) multiple sequence alignment tool.

FIG. 14A-14E shows that the combination of up to six modRNA components demonstrated robust neutralizing antibody responses. Female BALB/c mice (N=10 per group) were immunized IM with varying doses of LNP-formulated modRNAs as co-formulated bivalent modRNAs (0.4 μg of RSVpreF A+B or hMPVpreF A+B, or PIV3 preF+HN), co-formulated quadrivalent (abbreviated as “quad.” at 0.8 μg) with RSVpreF A+B and hMPVpreF A+B, a mixture of co-formulated bivalent RSVpreF A+B and hMPVpreF A+B (abbreviated as “bi.x2” at 0.8 μg 1:1 ratio or at 1.2 μg 1:2 ratio), co-formulated hexavalent (abbreviated as “hexa.” at 1.2 μg), or a mixture of co-formulated bivalent RSV preF A+B, hMPVpreF A+B and PIV3 preF+HN (abbreviated as “bi.x3” at 1.2 μg 1:1 ratio or at 1.6 μg 1:2 ratio) across two independent studies. Mice were vaccinated on a two-dose schedule at Day 0 (DO) and Day 21 (D21) and serum was collected on Day 35 (D35/2 weeks post-dose 2). Virus neutralization responses against (FIG. 14A) RSV A, (FIG. 14B) RSV B, (FIG. 14C) hMPV A, (FIG. 14D) hMPV B or (FIG. 14E) PIV3 were assessed by MNT assay using serum from D35. LOD is defined at 20 and error bars represent 95% confidence interval of the geometric means.

FIG. 15A-15D show that the novel LNP formulation (LNP2) induced strong neutralizing antibody and Th1-biased CD4+ T-cell responses and a robust CD8+ T-cell response. (FIG. 15A) Virus neutralization responses against RSV A preF were measured by MNT assay using serum from Day 35. LOD is defined at 20 and error bars represent 95% confidence interval of the geometric means. (FIG. 15B) Total % of RSV F antigen-specific IFN-γ+ CD4+ T-cells, (FIG. 15C) Total % of RSV F antigen-specific IL-4+CD4+ Tcells across selected groups, and (FIG. 15D) Total % of RSV F antigen-specific IFN-γ+ CD8+ T-cells across selected groups were assessed from splenocytes of selected groups (n=5/group) by ICS assay. Error bars represent the standard deviation of the means in T-cell analysis.

FIG. 16A-16E shows study results wherein female BALB/c mice (N=10 per group) were immunized IM with 1.2 μg (1:1:1:1:1:1 mRNA weight ratio, 0.2 μg per component) or 0.3 μg (1:1:1:1:1:1 mRNA weight ratio, 0.05 μg per component) LNP1-formulated or LNP2-formulated hexavalent modRNAs consisting of RSVpreF A+B, hMPVpreF A+B, and PIV3preF+HN. Mice were vaccinated on a two-dose schedule at Day 0 (DO) and Day 21 (D21) and serum was collected on Day 35 (D35/2 weeks post-dose 2). Virus neutralizing titers against (FIG. 16A) RSV A, (FIG. 16B) RSV B, (FIG. 16C) hMPV A, (FIG. 16D) hMPV B, or (FIG. 16E) PIV3 were assessed by MNT assay using serum from D35. LOD is defined at 20 and error bars represent 95% confidence interval of the geometric means.

FIG. 17A-17C. show lung and nasal turbinates viral loads after live RSV, hMPV or PIV3 challenge. Cotton rats (N=10) were vaccinated IM at Days 0 and 28 as with 45 μg of RSV/hMPV/PIV3 modRNA formulated in LNP1, 45 μg of RSV/hMPV/PIV3 modRNA formulated in LNP2, the original FI-RSV Lot 100 (formalin-inactivated RSV) at 1:100, FI-mock 1:100 or with matrix control (Mock). A control group received IN with live RSV, hMPV or PIV3 on Day 0. At Day 49 animals were challenged with infectious RSV (FIG. 17A), hMPV (FIG. 17B), or PIV3 (FIG. 17C). Lungs and nasal turbinates were harvested 5 days post-challenge for RSV and hMPV challenged animals. The same tissues were harvested 4 days post-challenge for PIV3 challenged animals. Five days (for RSV and hMPV) or four days (for PIV3) post-challenge virus shedding was assayed from i) lungs and ii) nasal turbinates using a standard plaque assay. Data are normalized to plaque forming units per gram of tissue (pfu/g). Black bar in each group represents the geometric mean pfu/g.

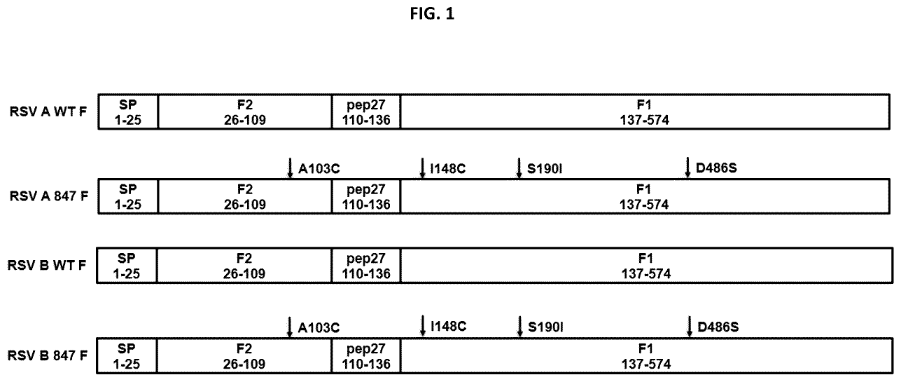

### DETAILED DESCRIPTION

The present disclosure provides for an RNA molecule (e.g., RNA polynucleotide) comprising at least one open reading frame (ORF) encoding a Metapneumovirus (hMPV) antigen and at least one untranslated region (UTR) selected from Table 22. In some aspects, the hMPV antigen is a hMPV polypeptide. In some aspects, the hMPV polypeptide is a hMPV F polypeptide. In some aspects, the hMPV polypeptide comprises an amino acid sequence set forth in Table 4. In some aspects, the RNA molecules comprise an ORF transcribed from at least one DNA nucleic acid sequence of Table 5. In some aspects, the RNA molecules comprise an ORF comprising an RNA nucleic acid sequence of Table 6. In some aspects the RNA molecule comprises at least one of a 5′ cap, 5′ UTR, 3′ UTR and poly-A tail. In other aspects the RNA molecule comprises at least one of a 5′ cap, 3′ UTR and poly-A tail. The present disclosure provides for an RNA molecule comprising modified nucleotides (e.g., modified RNA; modRNA). In some aspects, RNA molecule comprises at least one open reading frame encoding a hMPV fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396. In another aspect, the 5′ UTR comprises a nucleic acid sequence at least 92% identical to a nucleic acid sequence selected from the group consisting of SEQ ID NOs: 336, 341, 356-358, 372-375, and 378-396. In another aspect, the 5′ UTR comprises a nucleic acid sequence selected from the group consisting of:

SEQ ID NO: 373
(5UTR_563);
SEQ ID NO: 380
(5UTR_582);
and
SEQ ID NO: 385
(5UTR_599).

The present disclosure provides for an RNA molecule (e.g., RNA polynucleotide) comprising at least one open reading frame (ORF) encoding a parainfluenza virus type 3 (PIV3) antigen and at least one untranslated region (UTR) selected from Table 22. In some aspects, the PIV3 antigen is a PIV3 polypeptide. In some aspects, the PIV3 polypeptide is a PIV3 F polypeptide. In some aspects, the PIV3 F polypeptide comprises an amino acid sequence set forth in Table 13. In some aspects, the RNA molecules comprise an ORF transcribed from at least one DNA nucleic acid sequence of Table 14. In some aspects, the RNA molecules comprise an ORF comprising an RNA nucleic acid sequence of Table 15. In some aspects the RNA molecule comprises at least one of a 5′ cap, 5′ UTR, 3′ UTR and poly-A tail. In other aspects the RNA molecule comprises at least one of a 5′ cap, 3′ UTR and poly-A tail. The present disclosure provides for an RNA molecule comprising modified nucleotides (e.g., modified RNA; modRNA). In some aspects, RNA molecule comprises at least one open reading frame encoding a PIV3 fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396. In another aspect, the 5′ UTR comprises a nucleic acid sequence at least 92% identical to a nucleic acid sequence selected from the group consisting of SEQ ID NOs: 336, 341, 356-358, 372-375, and 378-396. In another aspect, the 5′ UTR comprises a nucleic acid sequence selected from the group consisting of:

SEQ ID NO: 373
(5UTR_563);
SEQ ID NO: 380
(5UTR_582);
and
SEQ ID NO: 385
(5UTR_599).

In some aspects, the PIV3 polypeptide is a PIV3 HN polypeptide. In some aspects, the PIV3 HN polypeptide comprises an amino acid sequence set forth in Table 16. In some aspects, the RNA molecules comprise an ORF transcribed from at least one DNA nucleic acid sequence of Table 17. In some aspects, the RNA molecules comprise an ORF comprising an RNA nucleic acid sequence of Table 18. In some aspects the RNA molecule comprises at least one of a 5′ cap, 5′ UTR, 3′ UTR and poly-A tail. In other aspects the RNA molecule comprises at least one of a 5′ cap, 3′ UTR and poly-A tail. The present disclosure provides for an RNA molecule comprising modified nucleotides (e.g., modified RNA; modRNA). In some aspects, RNA molecule comprises at least one open reading frame encoding a PIV3 hemagglutinin-neuraminidase protein (HN) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396. In another aspect, the 5′ UTR comprises a nucleic acid sequence at least 92% identical to a nucleic acid sequence selected from the group consisting of SEQ ID NOs: 336, 341, 356-358, 372-375, and 378-396. In another aspect, the 5′ UTR comprises a nucleic acid sequence selected from the group consisting of:

SEQ ID NO: 373
(5UTR_563);
SEQ ID NO: 380
(5UTR_582);
and
SEQ ID NO: 385
(5UTR_599).

The present disclosure provides for an RNA molecule (e.g., RNA polynucleotide) comprising at least one open reading frame (ORF) encoding a parainfluenza virus type 1 (PIV1) antigen and at least one untranslated region (UTR) selected from Table 22. In some aspects, the PIV1 antigen is a PIV1 polypeptide. In some aspects, the PIV1 polypeptide is a PIV1 F polypeptide. In some aspects, the PIV1 polypeptide comprises an amino acid sequence set forth in Table 7. In some aspects, the RNA molecules comprise an ORF transcribed from at least one DNA nucleic acid sequence of Table 8. In some aspects, the RNA molecules comprise an ORF comprising an RNA nucleic acid sequence of Table 9. In some aspects the RNA molecule comprises at least one of a 5′ cap, 5′ UTR, 3′ UTR and poly-A tail. In other aspects the RNA molecule comprises at least one of a 5′ cap, 3′ UTR and poly-A tail. The present disclosure provides for an RNA molecule comprising modified nucleotides (e.g., modified RNA; modRNA). In some aspects, RNA molecule comprises at least one open reading frame encoding a PIV1 fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396. In another aspect, the 5′ UTR comprises a nucleic acid sequence at least 92% identical to a nucleic acid sequence selected from the group consisting of SEQ ID NOs: 336, 341, 356-358, 372-375, and 378-396. In another aspect, the 5′ UTR comprises a nucleic acid sequence selected from the group consisting of:

SEQ ID NO: 373
(5UTR_563);
SEQ ID NO: 380
(5UTR_582);
and
SEQ ID NO: 385
(5UTR_599).

In some aspects, the PIV1 polypeptide is a PIV1 HN polypeptide. In some aspects, the PIV1 HN polypeptide comprises an amino acid sequence set forth in Table 10. In some aspects, the RNA molecules comprise an ORF transcribed from at least one DNA nucleic acid sequence of Table 11. In some aspects, the RNA molecules comprise an ORF comprising an RNA nucleic acid sequence of Table 12. In some aspects the RNA molecule comprises at least one of a 5′ cap, 5′ UTR, 3′ UTR and poly-A tail. In other aspects the RNA molecule comprises at least one of a 5′ cap, 3′ UTR and poly-A tail. The present disclosure provides for an RNA molecule comprising modified nucleotides (e.g., modified RNA; modRNA). In some aspects, RNA molecule comprises at least one open reading frame encoding a PIV1 hemagglutinin-neuraminidase protein (HN) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396. In another aspect, the 5′ UTR comprises a nucleic acid sequence at least 92% identical to a nucleic acid sequence selected from the group consisting of SEQ ID NOs: 336, 341, 356-358, 372-375, and 378-396. In another aspect, the 5′ UTR comprises a nucleic acid sequence selected from the group consisting of:

SEQ ID NO: 373
(5UTR_563);
SEQ ID NO: 380
(5UTR_582);
and
SEQ ID NO: 385
(5UTR_599).

The present disclosure provides for an immunogenic composition comprising any one of the RNA molecules encoding a polypeptide described hereinabove complexed with, encapsulated in, or formulated with one or more lipids, and forming lipid nanoparticles (RNA-LNPs).

The present disclosure provides for an immunogenic composition comprising an RNA molecule (e.g., RNA polynucleotide) comprising at least one open reading frame (ORF) encoding a respiratory syncytial virus (RSV) antigen and any one or more of the RNA molecules encoding a hMPV, PIV3 and/or PIV1 polypeptide described hereinabove, and at least one untranslated region (UTR) selected from Table 22. In some aspects, the RSV antigen is a RSV polypeptide. In some aspects, the RSV polypeptide is a RSV F polypeptide. In some aspects, the RSV polypeptide comprises an amino acid sequence set forth in Table 1. In some aspects, the RNA molecules comprise an ORF transcribed from at least one DNA nucleic acid sequence of Table 2. In some aspects, the RNA molecules comprise an ORF comprising an RNA nucleic acid sequence of Table 3. In some aspects the RNA molecule comprises at least one of a 5′ cap, 5′ UTR, 3′ UTR and poly-A tail. In other aspects the RNA molecule comprises at least one of a 5′ cap, 3′ UTR and poly-A tail. The present disclosure provides for an RNA molecule comprising modified nucleotides (e.g., modified RNA; modRNA). In some aspects, RNA molecule comprises at least one open reading frame encoding a respiratory syncytial virus (RSV) fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396. In another aspect, the 5′ UTR comprises a nucleic acid sequence at least 92% identical to a nucleic acid sequence selected from the group consisting of SEQ ID NOs: 336, 341, 356-358, 372-375, and 378-396. In another aspect, the 5′ UTR comprises a nucleic acid sequence selected from the group consisting of: 35

SEQ ID NO: 373
(5UTR_563);
SEQ ID NO: 380
(5UTR_582);
and
SEQ ID NO: 385
(5UTR_599).

The present disclosure further provides for an immunogenic composition comprising any one of the RNA molecules comprising at least one RNA nucleic acid described herein complexed with, encapsulated in, or formulated with one or more lipids, and forming RNA-LNPs. The present disclosure further provides for a method of preventing, treating or ameliorating an infection, disease or condition (e.g., RSV, hMPV and/or PIV3 and/or PIV1 infection-induced acute respiratory tract illness, lower respiratory tract illness, or lower respiratory tract disease, including pneumonia and bronchitis) in a subject via administering to a subject an effective amount of an RNA molecule, RNA-LNP or an immunogenic composition described herein. The present disclosure further provides for the use of the RNA molecule, RNA-LNP and/or an immunogenic composition described herein as a vaccine.

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary Embodiments (E) of the invention are set forth below:
E1. An RNA molecule comprising at least one open reading frame encoding a human Metapneumovirus (hMPV) fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.
E2. The RNA molecule of embodiment E1, wherein the hMPV F polypeptide encoded is a full-length or truncated hMPV F or a variant thereof.
E3. The RNA molecule of any one of embodiments E1-E2, wherein the hMPV F polypeptide comprises at least one amino acid mutation.
E4. The RNA molecule of embodiment E3, wherein the hMPV F polypeptide comprises at least one mutation relative to the wild-type hMPV F polypeptide, wherein the mutation is selected from:
   (1) Q100R, S101R, and D185P;
   (2) Q100R, S101R, L110C, T127C, A140C, A147C, N153C, D185P, L219K, V231I, N322C, T365C, E453Q, and V463C;
   (3) V84C, A140C, A147C, D185P, A249C, D454C, and V458C, and having deletions of amino acids at positions 89-112 replaced with a GSGGSG linker beginning at position 89;
   (4) Q100R, S101R, L110C, T127C, A140C, A147C, N153C, A185P, L219K, V231I, N322C, T365C, E453Q, and V463C; or
   (5) V84C, A140C, A147C, A185P, A249C, D454C, and V458C, and having deletions of amino acids at positions 89-112 replaced with a GSGGSG linker beginning at position 89, wherein the amino acid positions correspond to the amino acid sequence set forth in SEQ ID NO: 639.
E5. The RNA molecule of embodiment E4, wherein the hMPV F polypeptide is truncated after position 489 and linked to a T4 Fibritin Foldon domain.
E6. The RNA molecule of any one of embodiments E1-E5, wherein the hMPV F polypeptide is a prefusion F polypeptide.
E7. The RNA molecule of any one of embodiments E1-E6, wherein the hMPV polypeptide is of subtype A.
E8. The RNA molecule of any one of embodiments E1-E6, wherein the hMPV polypeptide is of subtype B.
E9. The RNA molecule of any one of embodiments E1-E8, wherein the hMPV F polypeptide has at least 90%, 95%, 96%, 97%, 98% or 99% identity to an amino acid sequence selected from SEQ ID NO: 639-647, 715, 716 or 721.
E10. The RNA molecule of embodiment E9, wherein the hMPV F polypeptide is selected from the group consisting of:
   (a) hMPV subtype A having an amino acid sequence set forth in SEQ ID NOs: 641-643, 646 or 715; and
   (b) hMPV subtype B having an amino acid sequence set forth in SEQ ID NOS: 644, 645, 647, 716 or 721,
   or a combination thereof.
E11. The RNA molecule of embodiment E10, wherein the hMPV F polypeptide has an amino acid sequence selected from the group consisting of SEQ ID NOs: 641-645 and 721.
E12. The RNA molecule of any one of embodiments E1-E11, wherein the open reading frame is transcribed from a DNA molecule comprising a nucleotide sequence having at least 90%, 95%, 96%, 97%, 98%, or 99% identity to any one of the sequences of SEQ ID NOs: 648-654, 659, 660, 717, 718 or 722.
E13. The RNA molecule of embodiment E12, wherein the open reading frame is transcribed from a DNA molecule comprising a nucleotide sequence selected from the group consisting of SEQ ID NOs: 649-651, 653-654, 659-660, 717, 718 and 722.
E14. The RNA molecule of embodiment E13, wherein the open reading frame is transcribed from a DNA molecule comprising a nucleotide sequence selected from the group consisting of SEQ ID NOs: 649-651, 653-654 and 722.
E15. The RNA molecule of embodiment E14, wherein the open reading frame is transcribed from a DNA molecule comprising a nucleotide sequence selected from SEQ ID NO: 651 or SEQ ID NO: 654.
E16. The RNA molecule of any one of embodiments E1-E15, wherein the open reading frame comprises a nucleic acid sequence having at least 90%, 95%, 96%, 97%, 98%, or 99% identity to any one of the sequences of SEQ ID NOs: 661-665, 670-671, 719-720, 723, 764 or 765.
E17. The RNA molecule of embodiment E16, wherein the open reading frame comprises a nucleic acid sequence selected from the group consisting of SEQ ID NOs: 661-665, 670-671, 719-720 and 723.
E18. The RNA molecule of embodiment E17, wherein the open reading frame comprises a nucleic acid sequence selected from the group consisting of SEQ ID NOs: 661-665 and 723.
E19. The RNA molecule of embodiment E18, wherein the open reading frame comprises a nucleic acid sequence selected from SEQ ID NO: 663 or SEQ ID NO: 665.
E20. An RNA molecule comprising at least one open reading frame encoding a parainfluenza virus type 3 (PIV3) fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.
E21. The RNA molecule of embodiment E20, wherein the PIV3 F polypeptide encoded is a full-length or truncated PIV3 F or a variant thereof.
E22. The RNA molecule of any one of embodiments E20 or E21, wherein the PIV3 F polypeptide comprises at least one amino acid mutation.
E23. The RNA molecule of embodiment E22, wherein the PIV3 F polypeptide comprises at least one mutation relative to the wild-type PIV3 F polypeptide, wherein the mutation is selected from:
   (1) E209C and L234C;
   (2) S160C, V170C, E209C, L234C, A463L, and S470L; or
   (3) Q162C, L168C, I213C, G230C, A463V, and I474Y, wherein the amino acid positions correspond to the amino acid sequence set forth in SEQ ID NO: 690.
E24. The RNA molecule of embodiment E23, wherein the PIV3 F polypeptide is truncated and linked to a T4 Fibritin Foldon domain.
E25. The RNA molecule of any one of embodiments E20-E24, wherein the PIV3 F polypeptide is a prefusion F polypeptide.
E26. The RNA molecule of any one of embodiments E20-E25, wherein the PIV3 F polypeptide has at least 90%, 95%, 96%, 97%, 98% or 99% identity to an amino acid sequence selected from SEQ ID NO: 690-694 and 712.
E27. The RNA of embodiment E26, wherein the PIV3 F polypeptide has an amino acid sequence selected from the group consisting of SEQ ID NOs: 691-694 and 712.
E28. The RNA molecule of embodiment E27, wherein the PIV3 F polypeptide has an amino acid sequence selected from the group consisting of SEQ ID NOs: 691, 692 and 712.
E29. The RNA molecule of any one of embodiments E20-E28, wherein the open reading frame is transcribed from a DNA molecule comprising a nucleotide sequence having at least 90%, 95%, 96%, 97%, 98%, or 99% identity to any one of the sequences of SEQ ID NOS: 695-697, 702-703 and 713.
E30. The RNA molecule of embodiment E29, wherein the open reading frame is transcribed from a DNA molecule comprising a nucleotide sequence selected from the group consisting of SEQ ID NOs: 696-697, 702-703 and 713.
E31. The RNA molecule of embodiment E30, wherein the open reading frame is transcribed from a DNA molecule comprising a nucleotide sequence selected from the group consisting of SEQ ID NOs: 696-697, 702-703 and 713.
E32. The RNA molecule of embodiment E31, wherein the open reading frame is transcribed from a DNA molecule comprising a nucleotide sequence selected from SEQ ID NO: 696 or SEQ ID NO: 697.
E33. The RNA molecule of any one of embodiments E20-E32, wherein the open reading frame comprises a nucleic acid sequence having at least 90%, 95%, 96%, 97%, 98%, or 99% identity to any one of the sequences of SEQ ID NOs: 704-705, 710-711, 714 or 766.
E34. The RNA molecule of embodiment E33, wherein the open reading frame comprises a nucleic acid sequence selected from the group consisting of SEQ ID NOs: 704-705, 710-711 and 714.
E35. The RNA molecule of embodiment E34, wherein the open reading frame comprises a nucleic acid sequence selected from the group consisting of SEQ ID NOs: 704, 705 and 714.
E36. The RNA molecule of embodiment E35, wherein the open reading frame comprises a nucleic acid sequence selected from SEQ ID NO: 704 or SEQ ID NO: 705.
E37. An RNA molecule comprising at least one open reading frame encoding a parainfluenza virus type 3 (PIV3) hemagglutinin-neuraminidase protein (HN) polypeptide.
E38. The RNA molecule of embodiment E37, wherein the PIV3 HN polypeptide encoded is a full-length or truncated PIV3 HN or a variant thereof.
E39. The RNA molecule of any one of embodiments E37 or E38, wherein the PIV3 HN polypeptide comprises at least one amino acid mutation.
E40. The RNA molecule of embodiment E39, wherein the PIV3 HN polypeptide comprises at least one mutation relative to the wild-type PIV3 HN polypeptide, wherein the mutation is selected from:
   i) a deletion of residues at positions 59-88; or
   ii) a deletion of residues at positions 59-130,
   wherein the amino acid positions correspond to the amino acid sequence set forth in SEQ ID NO: 724.
E41. The RNA molecule of any one of embodiments E37-E40, wherein the PIV3 HN polypeptide has at least 90%, 95%, 96%, 97%, 98% or 99% identity to an amino acid sequence selected from SEQ ID NO: 724, 725, 726, or 745-749.
E42. The RNA of embodiment E41, wherein the PIV3 HN polypeptide has an amino acid sequence selected from the group consisting of SEQ ID NOs: 725 and SEQ ID NO:726.
E43. The RNA molecule of any one of embodiments E37-E42, wherein the open reading frame is transcribed from a DNA molecule comprising a nucleotide sequence having at least 90%, 95%, 96%, 97%, 98%, or 99% identity to any one of the sequences of SEQ ID NOs: 727, 728 or 729.
E44. The RNA molecule of embodiment E43, wherein the open reading frame is transcribed from a DNA molecule comprising a nucleotide sequence selected from the group consisting of SEQ ID NO: 728 and 729.
E45. The RNA molecule of any one of embodiments E37-E44, wherein the open reading frame comprises a nucleic acid sequence having at least 90%, 95%, 96%, 97%, 98%, or 99% identity to any one of the sequences of SEQ ID NOs: 736, 737 or 738.
E46. The RNA molecule of embodiment E45, wherein the open reading frame comprises a nucleic acid sequence selected from the group consisting of SEQ ID NOs: 737 and 738.
E47. The RNA molecule of any one of embodiments E37-E46 further comprising a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.
E48. An RNA molecule comprising at least one open reading frame encoding a parainfluenza virus type 1 (PIV1) fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.
E49. The RNA molecule of embodiment E48, wherein the PIV1 F polypeptide encoded is a full-length or truncated PIV1 F or a variant thereof.
E50. The RNA molecule of any one of embodiments E48-E49, wherein the PIV1 F polypeptide comprises at least one mutation.
E51. The RNA molecule of embodiment E50, wherein the PIV1 F polypeptide comprises at least one mutation relative to the wild-type PIV1 F polypeptide, wherein the mutation is selected from:
   (1) Q92C, F113G, F114S, G134C, A466L, S473L, and A480L; or
   (2) F113G, F114S, G134A, A466L, and S473L,
   wherein the amino acid positions correspond to the amino acid sequence set forth in SEQ ID NO: 672.
E52. The RNA molecule of embodiment E51, wherein the PIV1 F polypeptide is truncated and linked to a T4 Fibritin Foldon domain.
E53. The RNA molecule of any one of embodiments E48-E52, wherein the PIV1 F polypeptide is a prefusion F polypeptide.
E54. The RNA molecule of any one of embodiments E48-E53, wherein the PIV1 F polypeptide has at least 90%, 95%, 96%, 97%, 98% or 99% identity to an amino acid sequence selected from SEQ ID NO: 672-676.
E55. The RNA molecule of embodiment E54, wherein the PIV1 F polypeptide has an amino acid sequence selected from the group consisting of SEQ ID NOs: 673-676.
E56. The RNA molecule of embodiment E55, wherein the PIV1 F polypeptide has an amino acid sequence selected from the group consisting of SEQ ID NOs: 673 and 674.
E57. The RNA molecule of any one of embodiments E48-E56, wherein the open reading frame is transcribed from a DNA molecule comprising a nucleotide sequence having at least 90%, 95%, 96%, 97%, 98%, or 99% identity to any one of the sequences of SEQ ID NOS: 677-679 682, or 683.
E58. The RNA molecule of embodiment E57, wherein the open reading frame is transcribed from a DNA molecule comprising a nucleotide sequence selected from the group consisting of SEQ ID NOs: 678-679, 682 or 683.
E59. The RNA molecule of embodiment E58, wherein the open reading frame is transcribed from a DNA molecule comprising a nucleotide sequence selected from the group consisting of SEQ ID NOs: 678 and 679.
E60. The RNA molecule of any one of embodiments E48-E59, wherein the open reading frame comprises a nucleic acid sequence having at least 90%, 95%, 96%, 97%, 98%, or 99% identity to any one of the sequences of SEQ ID NOs: 684-685, 688-689 or 767.
E61. The RNA molecule of embodiment E60, wherein the open reading frame comprises a nucleic acid sequence selected from the group consisting of SEQ ID NOs: 684, 685, 688 and 689.
E62. The RNA molecule of embodiment E61, wherein the open reading frame comprises a nucleic acid sequence selected from the group consisting of SEQ ID NOs: 684 and 685.
E63. An RNA molecule comprising at least one open reading frame encoding a parainfluenza virus type 1 (PIV1) hemagglutinin-neuraminidase protein (HN) polypeptide.
E64. The RNA molecule of embodiment E63, wherein the PIV1 HN polypeptide encoded is a full-length or truncated PIV1 HN or a variant thereof.
E65. The RNA molecule of any one of embodiments E63 or E64, wherein the PIV1 HN polypeptide comprises at least one amino acid mutation.
E66. The RNA molecule of embodiment E65, wherein the PIV1 HN polypeptide comprises at least one mutation relative to the wild-type PIV1 HN polypeptide, wherein the mutation is selected from:
   i) a deletion of residues at positions 57-84; or
   ii) a deletion of residues at positions 57-129,
   wherein the amino acid positions correspond to the amino acid sequence set forth in SEQ ID NO: 750.
E67. The RNA molecule of any one of embodiments E63-E66, wherein the PIV1 HN polypeptide has at least 90%, 95%, 96%, 97%, 98% or 99% identity to an amino acid sequence selected from SEQ ID NO: 750-757.
E68. The RNA of embodiment E67, wherein the PIV1 HN polypeptide has an amino acid sequence selected from the group consisting of SEQ ID NOs: 756 and 757.
E69. The RNA molecule of any one of embodiments E63-E68, wherein the open reading frame is transcribed from a DNA molecule comprising a nucleotide sequence having at least 90%, 95%, 96%, 97%, 98%, or 99% identity to any one of the sequences of SEQ ID NOs: 758-760.
E70. The RNA molecule of embodiment E69, wherein the open reading frame is transcribed from a DNA molecule comprising a nucleotide sequence selected from the group consisting of SEQ ID NO: 759 and 760.
E71. The RNA molecule of any one of embodiments E63-E70, wherein the open reading frame comprises a nucleic acid sequence having at least 90%, 95%, 96%, 97%, 98%, or 99% identity to any one of the sequences of SEQ ID NOs: 761-763.
E72. The RNA molecule of embodiment E71, wherein the open reading frame comprises a nucleic acid sequence selected from the group consisting of SEQ ID NOs: 762 and 763.
E73. The RNA molecule of any one of embodiments E63-E72 further comprising a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.
E74. The RNA molecule of any one of embodiments E1-E73, wherein the 5′ UTR comprises a nucleic acid sequence at least 92% identical to a nucleic acid sequence selected from the group consisting of SEQ ID NOs: 357, 372-374, and 378, 380 and 384.
E75. The RNA molecule of any one of embodiment E1 to E74, wherein the 5′ UTR comprises a nucleic acid sequence at least 95% identical to a nucleic acid sequence selected from the group consisting of SEQ ID NOs: 357, 372-374, 378, 380, and 384.
E76. The RNA molecule of any one of embodiments E1-E75, wherein the 5′ UTR comprises a nucleic acid sequence at least 98% identical to a nucleic acid sequence selected from the group consisting of SEQ ID NOs: 357, 372-374, 378, 380, and 384.
E77. The RNA molecule of any one of embodiments E1-E76, wherein the 5′ UTR comprises a nucleic acid sequence at least 98% identical to a nucleic acid sequence selected from the group consisting of SEQ ID NOs: 357, 372, 373, 378, 380, and 384.
E78. The RNA molecule of any one of embodiments E1-E77, wherein the 5′ UTR is selected from the group consisting of:
   (a) SEQ ID NOs: 357, 378, 380, and 384 for hMPV;
   (b) SEQ ID NOs: 357, 372, 380, and 384 for PIV1; and
   (c) SEQ ID NOs: 357, 373, and 380 for PIV3.
E79. The RNA molecule of any one of embodiments E1-E78, wherein the RNA molecule further comprises a 3′ untranslated region (3′ UTR).
E80. The RNA molecule of embodiment E79, wherein the 3′ UTR comprises nucleotides having sequence SEQ ID NO: 567 (3UTR_2 (hHBB)).
E81. The RNA molecule of any one of embodiments E1-E80, wherein the RNA molecule further comprises a 5′ cap moiety or a 3′ poly-A tail.
E82. The RNA molecule of embodiment E81, wherein the poly-A tail comprises about 20, 40, 60, 80 or 100 adenosines.
E83. The RNA molecule of any one of embodiments E1-E82, wherein the RNA comprises a 5′-cap structure, preferably m7G, cap0, cap1, cap2, a modified cap0 or a modified cap1 structure, preferably a 5′-cap1 structure.
E84. The RNA molecule of embodiment E83, wherein the 5′cap comprises

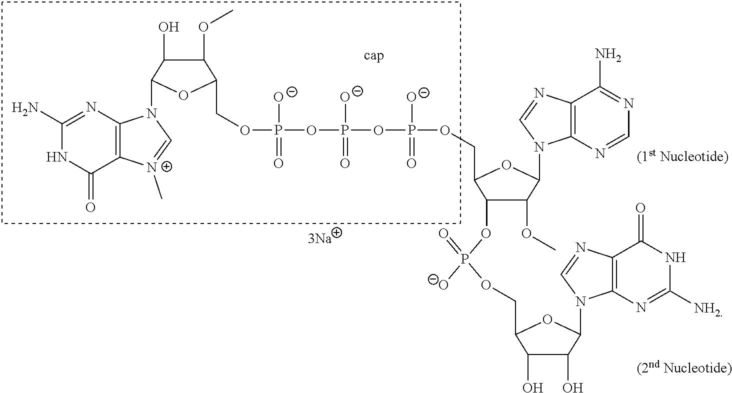 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

E85. The RNA molecule of any one of embodiments E1-E84, wherein the RNA molecule is codon-optimized.
E86. The RNA molecule of embodiment E1, wherein the hMPV F RNA molecule is transcribed from a DNA comprising a nucleotide sequence selected from the group consisting of SEQ ID NOs: 655-658.
E87. The RNA molecule of embodiment E1, wherein the hMPV F RNA molecule comprises a nucleic acid sequence selected from the group consisting of SEQ ID NOs: 666-669.
E88. The RNA molecule of embodiment E20, wherein the PIV3 F RNA molecule is transcribed from a DNA comprising a nucleotide sequence selected from the group consisting of SEQ ID NOs: 698-701.
E89. The RNA molecule of embodiment E20, wherein the PIV3 F RNA molecule comprises a nucleic acid sequence selected from the group consisting of SEQ ID NOs: 706-709. E90. The RNA molecule of embodiment E37, wherein the PIV3 HN RNA molecule is transcribed from a DNA comprising a nucleotide sequence selected from the group consisting of SEQ ID NOs: 730-735.
E91. The RNA molecule of embodiment E37, wherein the PIV3 HN RNA molecule comprises a nucleic acid sequence selected from the group consisting of SEQ ID NOs: 739-744.
E92. The RNA molecule of embodiment E48, wherein the PIV1 F RNA molecule is transcribed from a DNA comprising a nucleotide sequence selected from the group consisting of SEQ ID NOs: 680-681.
E93. The RNA molecule of embodiment E48, wherein the PIV1 F RNA molecule comprises a nucleic acid sequence selected from the group consisting of SEQ ID NOs: 686-687.
E94. The RNA molecule of embodiment E63, wherein the PIV1 HN RNA molecule is transcribed from a DNA comprising a nucleotide sequence selected from the group consisting of SEQ ID NO: 769, 770, 772 and 773.
E95. The RNA molecule of embodiment E63, wherein the PIV1 HN RNA molecule comprises a nucleic acid sequence selected from the group consisting of SEQ ID NO: 775, 776, 778 and 779.
E96. The RNA molecule of any one of embodiments E1-E95, wherein the open reading frame comprises a G/C content of at least 50%, 55%, 60%, 65%, 70%, or 75%, or of about 50% to 75% or 55% to 70%.
E97. The RNA molecule of any one of embodiments E1-E96, wherein the encoded polypeptide localizes in the cellular membrane, localizes in the Golgi and/or is secreted.
E98. The RNA molecule of any one of embodiments E1-E97, wherein the RNA molecule is mRNA.
E99. The RNA molecule of any one of embodiments E1-E98, wherein the RNA molecule comprises at least one modified nucleotide.
E100. The RNA molecule of embodiment E99, wherein all the uridines are replaced by a modified nucleotide.
E101. The RNA molecule of embodiment E99 or E100, wherein the modified nucleotide is pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine or 2′-O-methyl uridine.
E102. The RNA molecule of embodiment E101, wherein the modified nucleotide is N1-methyl pseudouridine (m1ψ).
E103. The RNA molecule of embodiment E101, wherein the modified nucleotide is pseudouridine (ψ).
E104. The RNA molecule according to any one of embodiments E1-E103, wherein the RNA has an integrity greater than 85%.
E105. The RNA molecule according to any one of embodiments E1-E104, wherein the RNA has a purity of greater than 85%.
E106. An immunogenic composition comprising at least one RNA molecule of any one of embodiments E1-E105, wherein the RNA molecule is formulated in a lipid nanoparticle (LNP) thereby forming an RNA-LNP.
E107. The immunogenic composition of embodiment E106, wherein the RNA molecule is selected from the group consisting of an RNA molecule encoding hMPV F A, hMPV F B, PIV3 F, PIV3 HN, PIV1 F and/or PIV1 HN.
E108. The immunogenic composition of embodiment E107, further comprising an RNA molecule comprising at least one open reading frame encoding a respiratory syncytial virus (RSV) fusion protein F (F) polypeptide and a 5′ untranslated region (RSV 5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.
E109. The immunogenic composition of embodiment E108, wherein the RSV 5′ UTR comprises a nucleic acid sequence at least 92% identical to a nucleic acid sequence selected from the group consisting of SEQ ID NOs: 336, 341, 356-358, 372-375, and 378-396.
E110. The immunogenic composition of any one of embodiments E108-E109, wherein the RSV 5′ UTR comprises a nucleic acid sequence at least 95% identical to a nucleic acid sequence selected from the group consisting of SEQ ID NOs: 336, 357, 372-374, 378, 380, 384 and 385.
E111. The immunogenic composition of any one of embodiments E108-E110, wherein the RSV 5′ UTR comprises a nucleic acid sequence at least 98% identical to a nucleic acid sequence selected from the group consisting of SEQ ID NOs: 357, 372-374, 378, 380, 384 and 385.
E112. The immunogenic composition of any one of embodiments E108-E111, wherein the RSV 5′ UTR comprises a nucleic acid sequence at least 98% identical to a nucleic acid sequence selected from the group consisting of SEQ ID NOs: 357, 373, 378, 380, 384 and 385.
E113. The immunogenic composition of any one of embodiments E108-E112, wherein the RSV 5′ UTR comprises a nucleic acid sequence selected from the group consisting of:

SEQ ID NO: 373
(5UTR_563);
SEQ ID NO: 380
(5UTR_582);
and
SEQ ID NO: 385
(5UTR_599).

E114. The immunogenic composition of any one of embodiments E108-E113, wherein the RNA molecule comprising at least one open reading frame encoding a RSV F further comprises a 3′ untranslated region (3′ UTR).
E115. The immunogenic composition of embodiment E114, wherein the 3′ UTR comprises nucleotides having sequence SEQ ID NO: 567 (3UTR_2 (hHBB)).
E116. The immunogenic composition of any one of embodiments E108-E115, wherein the RSV F polypeptide encoded is a full-length or truncated RSV F or a variant thereof.
E117. The immunogenic composition of any one of embodiments E108-E116, wherein the RSV F polypeptide comprises at least one mutation.
E118. The RNA molecule of embodiment E117, wherein the RSV F polypeptide comprises at least one mutation relative to the wild-type RSV F polypeptide, wherein the mutation is selected from:
   (1) A103C, I148C, S190I and D486S;
   (2) T54H, A103C, I148C, S190I, V296I and D486S; or
   (3) T54H S55C, L188C and D486S,
   wherein the amino acid positions correspond to the amino acid sequence set forth in SEQ ID NO: 1.
E119. The RNA molecule of embodiment E118, wherein the RSV F polypeptide is truncated and linked to a T4 Fibritin Foldon domain.
E120. The immunogenic composition of any one of embodiments E108-E119, wherein the RSV F polypeptide is a prefusion F polypeptide.
E121. The RNA molecule of any one of embodiments E108-E120, wherein the RSV F polypeptide is of subtype A.
E122. The RNA molecule of any one of embodiments E108-E120, wherein the RSV F polypeptide is of subtype B.
E123. The immunogenic composition of any one of embodiments E108-E122, wherein the RSV F polypeptide has at least 90%, 95%, 96%, 97%, 98% or 99% identity to an amino acid sequence selected from SEQ ID NO: 1-6 or 621-626.
E124. The immunogenic composition of embodiment E123, wherein the RSV F polypeptide is selected from:
   (a) RSV subtype A having an amino acid sequence set forth in SEQ ID NOs: 4, 621, 623 and 625;
   (b) RSV subtype B having an amino acid sequence set forth in SEQ ID NOs: 6, 622, 624 and 626,
   or a combination thereof.
E125. The immunogenic composition of embodiment E124, wherein the RSV F polypeptide has an amino acid sequence selected from the group consisting of SEQ ID NOs: 4 and 6.
E126. The immunogenic composition of any one of embodiments E108-E125, wherein the open reading frame encoding RSV F polypeptide is transcribed from a DNA comprising a nucleotide sequence having at least 90%, 95%, 96%, 97%, 98%, or 99% identity to any one of the sequences of SEQ ID NOs: 7-10 or 627-632.
E127. The immunogenic composition of embodiment E126, wherein the open reading frame encoding RSV F polypeptide is transcribed from a DNA comprising a nucleotide sequence selected from the group consisting of SEQ ID NOs: 8, 10, and 627-632.
E128. The immunogenic composition of embodiment E127, wherein the open reading frame encoding RSV F polypeptide is transcribed from a DNA comprising a nucleotide sequence selected from the group consisting of SEQ ID NOs: 8 and 10.
E129. The immunogenic composition of any one of embodiments E108-E128, wherein the open reading frame encoding RSV F polypeptide comprises a nucleic acid sequence having at least 90%, 95%, 96%, 97%, 98%, or 99% identity to any one of the sequences of SEQ ID NOs: 15-16 or 633-638 and 780-781.
E130. The immunogenic composition of embodiment E129, wherein the open reading frame encoding RSV F polypeptide comprises a nucleic acid sequence selected from the group consisting of SEQ ID NOs: 15-16 and 633-638.
E131. The immunogenic composition of embodiment E130, wherein the open reading frame encoding RSV F polypeptide comprises a nucleic acid sequence selected from the group consisting of SEQ ID NOs: 15 and 16.
E132. The immunogenic composition of any one of embodiments E108-E131, wherein the RNA molecule encoding RSV F polypeptide further comprises a 5′ cap moiety and/or a 3′ poly-A tail.
E133. The immunogenic composition of embodiment E132, wherein the poly-A tail comprises about 20, 40, 60, 80 or 100 adenosines.
E134. The immunogenic composition of embodiment E108, wherein the RNA molecule encoding RSV F polypeptide is transcribed from a DNA comprising a nucleotide sequence selected from the group consisting of SEQ ID NOs: 11-14.
E135. The immunogenic composition of embodiment E108, wherein the RNA molecule encoding RSV F polypeptide comprises a nucleic acid sequence selected from the group consisting of SEQ ID NOs: 17-20.
E136. The immunogenic composition of any one of embodiments E108-E135, wherein the open reading frame encoding RSV F polypeptide comprises a G/C content of at least 50%, 55%, 60%, 65%, 70%, or 75%, or of or of about 50% to 75% or 55% to 70%.
E137. The immunogenic composition of any one of embodiments E108-E136, wherein the encoded RSV F polypeptide localizes in the cellular membrane, localizes in the Golgi and/or is secreted.
E138. The immunogenic composition of any one of embodiments E108-E137, wherein the RNA molecule is mRNA.
E139. The immunogenic composition of any one of embodiments E108-E138, wherein the RNA molecule is codon-optimized.
E140. The immunogenic composition of any one of embodiments E108-E139, wherein the RNA molecule comprises at least one modified nucleotide.
E141. The immunogenic composition of embodiment E140, wherein all the uridines are replaced by a modified nucleotide.
E142. The immunogenic composition of embodiment E140 or E141, wherein the modified nucleotide is pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine or 2′-O-methyl uridine.
E143. The immunogenic composition of embodiment E142, wherein the modified nucleotide is N1-methylpseudouridine (m1ψ).
E144. The immunogenic composition of embodiment E142, wherein the modified nucleotide is pseudouridine (ψ).
E145. The immunogenic composition of according to any one of embodiments E108-E144, wherein the RNA has an integrity greater than 85%.
E146. The immunogenic composition of according to any one of embodiments E108-E145, wherein the RNA has a purity of greater than 85%.
E147. The immunogenic composition of any one of embodiments E106-E146, wherein the RNA molecules are formulated in one or more lipid nanoparticles (RNA-LNP).
E148. The immunogenic composition of any one of embodiments E106-E147, wherein the immunogenic composition comprises one or more RNA-LNPs selected from:
   i. an RNA-LNP comprising one RNA molecule,
   ii. an RNA-LNP comprising two or more co-formulated RNA molecules that do not encode the same antigen (pre-mixed), or
   iii. a mixture of two or more RNA-LNPs selected from (i) or (ii) (post-mix).
E149. The immunogenic composition of embodiment E148 comprising:
   i) a first and a second RNA molecule;
   ii) a first, a second and a third RNA molecule;
   iii) a first, a second, a third and a fourth RNA molecule;
   iv) a first, a second, a third, a fourth, and a fifth RNA molecule;
   v) a first, a second, a third, a fourth, a fifth, and a sixth RNA molecule;
   vi) a first, a second, a third, a fourth, a fifth, a sixth, and a seventh RNA molecule; or
   vii) a first, a second, a third, a fourth, a fifth, a sixth, a seventh, and an eighth RNA molecule,
   wherein the first, second, third, fourth, fifth, sixth, seventh and eighth RNA molecules of the immunogenic composition do not encode the same antigen and are formulated in one LNP or in separate LNPs.
E150. The immunogenic composition of embodiment E149, wherein the RNA molecules are present in about equal ratios.
E151. The immunogenic composition of embodiment E149, wherein the RNA molecules are not present in equal ratios.
E152. The immunogenic composition of any one of embodiments E148-E151, wherein the RNA-LNP comprises:
   a. bivalent RNA-LNPs comprising
      i. a first bivalent RNA-LNP comprising an RNA molecule encoding a first antigen and an RNA molecule encoding a second antigen,
      ii. a second bivalent RNA-LNP comprising an RNA molecule encoding a third antigen and an RNA molecule encoding a fourth antigen,
      iii. a third bivalent RNA-LNP comprising an RNA molecule encoding a fifth antigen and an RNA molecule encoding a sixth antigen, or
      iv. a fourth bivalent RNA-LNP comprising an RNA molecule encoding a seventh antigen and an RNA molecule encoding an eighth antigen,
   wherein the RNA molecules do not encode the same antigen and are co-formulated into LNPs (pre-mixed), and further wherein the weight ratio of the two RNA molecules in each bivalent RNA-LNP is 1:1;
   b. a quadrivalent RNA-LNP comprising an RNA molecule encoding a first antigen, an RNA molecule encoding a second antigen, an RNA molecule encoding a third antigen and an RNA molecule encoding a fourth antigen, wherein the first, second, third and fourth RNA molecules do not encode the same antigen and are coformulated into LNPs (pre-mixed), and further wherein the weight ratio of the first:second:third:fourth RNA molecules is 1:1:1:1;
   c. a mixture of RNA-LNPs comprising the first bivalent RNA-LNP of item (a) and the second bivalent RNA-LNP of item (b), wherein the RNA molecules of the first bivalent RNA-LNP and the second bivalent RNA-LNP do not encode the same antigen, and further wherein the ratio of the first bivalent RNA-LNP:second bivalent RNA-LNP is about 1:1 or 1:2;
   d. a hexavalent RNA-LNP comprising an RNA molecule encoding a first antigen, an RNA molecule encoding a second antigen, an RNA molecule encoding a third antigen, an RNA molecule encoding a fourth antigen, an RNA molecule encoding a fifth antigen and an RNA molecule encoding a sixth antigen, wherein the first, second, third, fourth, fifth and sixth RNA molecules do not encode the same antigen and are coformulated into LNPs (pre-mixed), and further wherein the weight ratio of the first:second:third:fourth:fifth:sixth RNA molecules is 1:1:1:1:1:1 or 1:1:2:2:1:1;
   e. a mixture of RNA-LNPs comprising the first bivalent RNA-LNP of item (a), the second bivalent RNA-LNP of item (a), and the third bivalent RNA-LNP of item (a), wherein the RNA molecules of the first, second and third bivalent RNA-LNPs do not encode the same antigen, further wherein the ratio of the first bivalent RNA-LNP:second bivalent RNA-LNP:third bivalent RNA-LNP is about 1:1:1 or 1:2:1;
   f. an octavalent RNA-LNP comprising an RNA molecule encoding a first antigen, an RNA molecule encoding a second antigen, an RNA molecule encoding a third antigen, an RNA molecule encoding a fourth antigen, an RNA molecule encoding a fifth antigen, an RNA molecule encoding a sixth antigen, an RNA molecule encoding a seventh antigen, and an RNA molecule encoding an eighth antigen wherein the first, second, third, fourth, fifth, sixth, seventh and eighth RNA molecules do not encode the same antigen and are coformulated into LNPs (pre-mixed), and further wherein the weight ratio of the first:second:third:fourth:fifth:sixth:seventh:eighth RNA molecules is 1:1:1:1:1:1:1:1 or 1:1:2:2:1:1:1:1; or
   g. a mixture of RNA-LNPs comprising the first bivalent RNA-LNP of item (a), the second bivalent RNA-LNP of item (a), the third bivalent RNA-LNP of item (a), and the fourth bivalent RNA-LNP of item (a), wherein the RNA molecules of the first, second, third and fourth bivalent RNA-LNPs do not encode the same antigen, and further wherein the ratio of the first bivalent RNA-LNP:second bivalent RNA-LNP:third bivalent RNA-LNP:fourth bivalent RNA-LNP is about 1:1:1:1 or 1:2:1:1.
E153. The immunogenic composition of embodiments E149-E152, wherein
   i. the first antigen is an hMPV preF A polypeptide;
   ii. the second antigen is an hMPV preF B polypeptide;
   iii. the third antigen is an RSV preF A polypeptide;
   iv. the fourth antigen is an RSV preF B polypeptide;
   v. the fifth antigen is a PIV3 preF polypeptide;
   vi. the sixth antigen is a PIV3 HN polypeptide;
   vii. the seventh antigen is a PIV1 F polypeptide; and
   viii. the eighth antigen is a PIV1 HN polypeptide.
E154. The immunogenic composition of embodiment E152 comprising bivalent LNPs wherein:
   i. the first bivalent RNA-LNP comprises an RNA molecule encoding RSV preF A and an RNA molecule encoding RSV preF B polypeptide; and/or
   ii. the second bivalent RNA-LNP comprises an RNA molecule encoding hMPV preF A and an RNA molecule encoding hMPV preF B polypeptide; and/or
   iii. the third bivalent RNA-LNP comprises an RNA molecule encoding PIV3 preF polypeptide and an RNA molecule encoding PIV3 HN polypeptide; and/or
   iv. the fourth bivalent RNA-LNP comprises an RNA molecule encoding PIV1 preF polypeptide and RNA molecule encoding PIV1 HN polypeptide.
E155. The immunogenic composition of embodiment E154, wherein
   i. the first bivalent RNA-LNP comprises the RNA molecule encoding RSV preF A and the RNA molecule encoding RSV preF B polypeptide set forth in embodiments E108-E131; and/or
   ii. the second bivalent RNA-LNP comprises the RNA molecule encoding hMPV preF A and the RNA molecule encoding hMPV preF B polypeptide set forth in embodiments E1-E19 or E86-E87; and/or
   iii. the third bivalent RNA-LNP comprises the RNA molecule encoding PIV3 preF polypeptide set forth in embodiments E20-E36 or E88-E89 and the RNA molecule encoding PIV3 HN polypeptide set forth in embodiments E37-E47 or E90-91; and/or
   iv. the fourth bivalent RNA-LNP comprises an RNA molecule encoding the PIV1 preF polypeptide set forth in embodiments E48-E62 or E92-E93 and the RNA molecule encoding PIV1 HN polypeptide set forth in embodiments E63-E72 or E94-E95.
E156. The immunogenic composition of any one of embodiments E106 to E155, wherein lipid nanoparticle comprises:
   (i) at least one of a steroid or steroid analog or a mixture of a steroid analog and a steroid,
   (ii) a neutral lipid,
   (iii) a PEGylated lipid, and
   (iv) a cationic lipid.
E157. The immunogenic composition of any one of embodiments E156, wherein the steroid is cholesterol.
E158. The immunogenic composition of embodiment E156, wherein the steroid analog is beta-sitosterol.
E159. The immunogenic composition of embodiment E156, wherein the mixture of a steroid analog and a steroid comprises a mixture of beta-sitosterol and cholesterol having a molar ratio of beta-sitosterol:cholesterol of 6:4.
E160. The immunogenic composition of embodiment 156-159, wherein the neutral lipid is distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoylphosphatidyethanol amine (SOPE), or 1,2-dielaidoyl-sn-glycero-3-phophoethanolamine (transDOPE).
E161. The immunogenic composition of embodiment E160, wherein the neutral lipid is 1,2-distearoyl-sn-glycero-3-phosphocholine (DSPC).
E162. The immunogenic composition of embodiments E156-E161, wherein the PEGylated lipid is PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols, 2-[(polyethylene glycol)-2000]-N,N-ditetradecylacetamide, glycol-lipids including PEG-c-DOMG, PEG-c-DMA, PEG-s-DMG, N-[(methoxy polyethylene glycol) 2000) carbamyl]-1,2-dimyristyloxlpropyl-3-amine (PEG-c-DMA), and PEG-2000-DMG, PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy (polyethoxy)ethyl-N-(2,3di(tetradecanoxy)propyl)carbamate or 2,3-di(tetradecanoxy)propyl-N-(u>-methoxy(polyethoxy)ethyl)carbamate.
E163. The immunogenic composition of embodiment E162, wherein the PEGylated lipid is 2-[(polyethylene glycol)-2000]-N,N-ditetradecylacetamide (ALC-0159) having the structure:

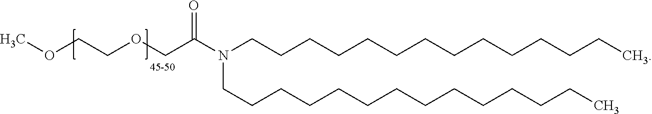

E164. The immunogenic composition of embodiments E156-E163, wherein the cationic lipid comprises N,N-dimethyl-2,3-dioleyloxy)propylamine (DODMA), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), (4-hydroxybutyl) azanediyl)bis(hexane-6,1-diyl)bis(2-hexyldecanoate) (ALC-0315), heptadecane-9-yl 8-((2-hydroxyethyl) (6-oxo-6-(undecyloxy)hexyl)amino) octanoate (SM-102), or a mixture thereof.
E165. The immunogenic composition of embodiment E164, wherein the cationic lipid is ALC-0315 (4-hydroxybutyl) azanediyl)bis(hexane-6,1-diyl)bis(2-hexyldecanoate) (ALC-0315) having the structure:

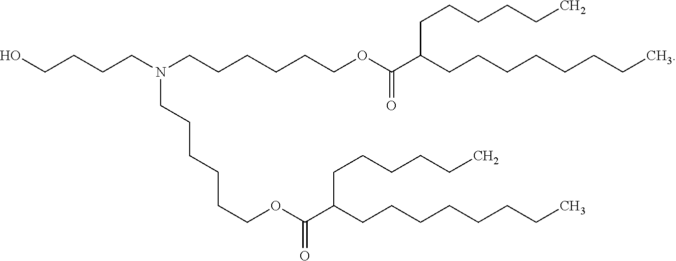 [SMILES: CCCCCCCCC(CCCCCC)C(=O)OCCCCCCN(CCCCO)CCCCCCOC(=O)C(CCCCCC)CCCCCCCC]

E166. The immunogenic composition of any one of embodiments E156-E163, wherein the cationic lipid is 2-hexyldecyl 6-[(2-{[4-(heptylcarbonylamino)butyl]-N-methylamino}ethyl)[5-(2-hexyldecyloxycarbonyl)pentyl]amino]hexanoate (ALC-0515) having the structure:

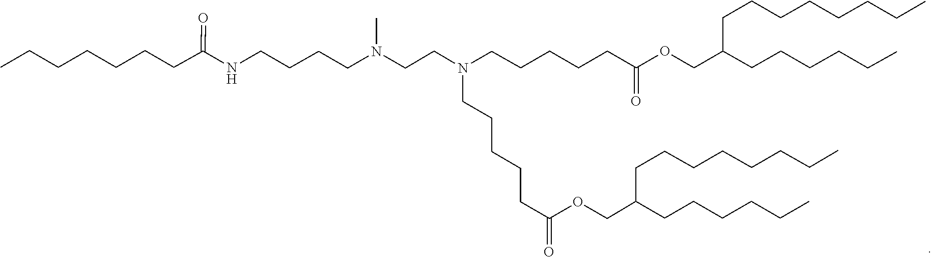 [SMILES: CCCCCCCCC(CCCCCC)COC(=O)CCCCCN(CCCCCC(=O)OCC(CCCCCC)CCCCCCCC)CCN(C)CCCCNC(=O)CCCCCCC]

E167. The immunogenic composition of embodiment E156-E166, wherein the LNP comprises:
   a) ALC-0315, cholesterol, DSPC, and ALC-0159;
   b) ALC-0315, beta-sitosterol, cholesterol, DSPC, and ALC-0159; or
   c) ALC-0515, beta-sitosterol, cholesterol, DSPC, and ALC-0159,
   wherein the molar ratio of beta-sitosterol:cholesterol is 6:4.
E168. The immunogenic composition according to any one of embodiments E106-E167, wherein at least 80% of the total RNA in the immunogenic composition is encapsulated.
E169. The immunogenic composition according to any one of embodiments E106-E168, wherein the percentage of intact mRNA encapsulated in the LNP is at least 80%.
E170. The immunogenic composition according to any one of embodiments E106-E169, wherein the LNP comprises an N:P ratio of from about 2:1 to about 30:1.
E171. The immunogenic composition of embodiment E170, wherein the LNP has an N:P ratio of at least 5.
E172. The immunogenic composition of embodiment E170, wherein the LNP has an N:P ratio of at least 6.
E173. The immunogenic composition of embodiment E170, wherein the LNP has an N:P ratio of 10.
E174. The immunogenic composition of embodiment E170, wherein the LNP has an N:P ratio of 6.
E175. The immunogenic composition of embodiments E106-E174, wherein the immunogenic composition further comprises a fatty acid, a derivative or salt thereof.

As used herein, the term “fatty acid” shall mean an aliphatic acid, consisting of a carboxylic acid functional group at the polar end and a hydrocarbon chain at the non-polar end of the fatty acid. The general structure for a fatty acid in mono form is:

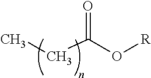

wherein R is a hydrogen atom or an ammonium, sodium, potassium, magnesium, or calcium cation. The chain lengths for fatty acids are 4 to 40 carbons in length (i.e. n is 2 to 38). Examples of fatty acids include, but are not limited to, oleic acid, arachidonic acid, eruric acid, linolenic acid, ricinoleic acid, palmitoleic acid, linoleic acid. A “fatty acid salt” e.g. sodium salt of oleic acid, also known as sodium oleate, is a compound formed when the carboxylic acid group of fatty acid is neutralized by sodium hydroxide (NaOH) or other neutralizing agent, essentially creating a salt with the chemical formula “CH₃(CH₂)7CH═CH(CH₂)7COONa” where “COONa” represents the sodium carboxylate group. Salts include, but are not limited to, sodium, potassium, magnesium or calcium.
E176. The immunogenic composition of embodiment E175, wherein the LNP comprises:
   a) ALC-0315, cholesterol, DSPC, ALC-0159 and a fatty acid or salt thereof;
   b) ALC-0315, beta-sitosterol, cholesterol, DSPC, ALC-0159 and a fatty acid or salt thereof; or
   c) ALC-0515, beta-sitosterol, cholesterol, DSPC, ALC-0159 and a fatty acid or salt thereof,
   wherein the molar ratio of beta-sitosterol:cholesterol is 6:4.
E177. The immunogenic composition according to any one of embodiments E175-E176, wherein the immunogenic composition comprises a fatty acid to RNA weight ratio (O:R) of from about 1.5:1 to about 24:1.
E178. The immunogenic composition of embodiment E177, wherein the immunogenic composition has an O:R of at least 1.5:1.
E179. The immunogenic composition of embodiment E178, wherein the immunogenic composition has an O:R selected from about 1.5:1, about 2:1, about 3:1, about 4:1, about 6:1, about 6.5:1, about 8:1, about 10:1, about 12:1, about 13:1 or about 24:1.
E180. The immunogenic composition of embodiment E179, wherein the immunogenic composition has an O:R of 8:1.
E181. The immunogenic composition of any of embodiments E175-E180, wherein the fatty acid is oleic acid.
E182. The immunogenic composition of any one of embodiments E175-E180, wherein the fatty acid salt is sodium oleate.
E183. The immunogenic composition of any one of embodiments E106-E182, wherein the LNP comprises about 15-60 mol % ionizable cationic lipid, about 13-60 mol % cholesterol or mixture of cholesterol and a cholesterol analog, about 3-30 mol % neutral lipid, and about 0.5-10 mol % polymer-conjugated lipid.
E184. The immunogenic composition according to any one of embodiments E106-E183, wherein the lipid nanoparticle comprises about 20 mol % to about 60 mol % ionizable cationic lipids, about 18.5 mol % to about 48.5 mol % mixture of cholesterol and a cholesterol analog, about 0 mol % to about 30 mol % neutral lipids, and about 0 mol % to about 10 mol % polymer-conjugated lipid.
E185. The immunogenic composition of any of embodiments E106-E184, wherein the immunogenic composition comprises a mol % ratio of cationic lipid:cholesterol analog:cholesterol:neutral lipid:polymer conjugated lipid:fatty acid or fatty acid salt selected from the group:
   a) 42.22:21.71:14.47:8.89:1.6:11.11;
   b) 40.71:20.93:13.95:8.57:1.54:14.29;
   c) 38:13.02:19.54:8:1.44:20;
   d) 35.62:18.31:12.21:7.5:1.35:25;
   e) 31.67:10.85:16.28:6.67:1.2:33.34;
   f) 30.81:10.56:15.84:6.49:1.17:35.14;
   g) 28.5:14.65:9.77:6:1.08:40;
   h) 25.91:13.32:8.88:5.45:0.98:45.46; or
   i) 22.8:11.72:7.81:4.8:0.86:52.
E186. The immunogenic composition according to any one of embodiments E106-E185, wherein the immunogenic composition further comprises a buffer.
E187. The immunogenic composition of embodiment E186, wherein the buffer is Tris.
E188. The immunogenic composition of embodiment E187, wherein the buffer is 10 mM Tris.
E189. The immunogenic composition according to any one of embodiment E186, wherein the buffer is phosphate buffered saline (PBS).
E190. The immunogenic composition according to any one of embodiments E106-E189, wherein the immunogenic composition further comprises sucrose.
E191. The immunogenic composition of embodiment E190, wherein the immunogenic composition comprises 300 mM sucrose.
E192. The immunogenic composition according to any one of embodiments 106-191, wherein the immunogenic composition has a pH from about 4 to about 11, from about 5 to about 10, from about 6 to about 9, from about 7 to about 8, about 7 to about 7.5, or from about 7.3 to about 7.5.
E193. The immunogenic composition according to embodiment E192, wherein the immunogenic composition has a pH of about 7.4.
E194. The immunogenic composition according to any one of embodiments E147-E193, wherein the immunogenic composition is frozen.
E195. The immunogenic composition according to any one of embodiments E175-E193, wherein the immunogenic composition has never been frozen.
E196. The immunogenic composition according to any one of embodiments E175-E193, wherein the immunogenic composition is liquid.
E197. The immunogenic composition according to any one of embodiments E175-E193, wherein the immunogenic composition is lyophilized.
E198. The immunogenic composition according to any one of embodiments E106-E197, wherein the immunogenic composition is administered in an effective amount to induce an immune response in a subject administered at least one dose of the vaccine.
E199. The immunogenic composition of embodiment E198, wherein the efficacy of the vaccine in vaccinated subjects is at least 60%, relative to unvaccinated subjects, following a single dose of the vaccine.
E200. The immunogenic composition of embodiment E199, wherein the efficacy of the vaccine in vaccinated subjects is at least 70%, relative to unvaccinated subjects, following a single dose of the vaccine.
E201. The immunogenic composition of embodiment E200, wherein the efficacy of the vaccine in vaccinated subjects is at least 80%, relative to unvaccinated subjects, following a single dose of the vaccine.
E202. The immunogenic composition of embodiment E201, wherein the efficacy of the vaccine in vaccinated subjects is at least 90%, relative to unvaccinated subjects, following a single dose of the vaccine.
E203. The immunogenic composition of embodiment E198-E202, wherein the effective amount is sufficient to produce protective levels of neutralizing antibody against the antigenic hMPV F protein and/or the antigenic PIV3 F protein and/or the antigenic PIV3 HN protein and/or the antigenic RSV F protein and/or the antigenic PIV1 F protein and/or the antigenic PIV1 HN protein as measured in serum of a subject vaccinated with at least one dose of the vaccine.

E204. The immunogenic composition of embodiment E198-E203, wherein an anti-hMPV F protein antibody titer and/or an anti-PIV3 F protein antibody titer and/or an anti-PIV3 HN protein antibody titer and/or an anti-RSV F protein antibody titer and/or an anti-PIV1 F protein antibody titer and/or an anti-PIV1 HN protein antibody titer produced in a subject vaccinated with at least one dose of the vaccine is increased by at least 2-fold to 10-fold relative to a control, wherein the control is an anti-hMPV F protein antibody titer and/or an 25 anti-PIV3 F protein antibody titer and/or an anti-PIV3 HN protein antibody titer and/or an RSV F protein antibody titer and/or a PIV1 F protein antibody titer and/or a PIV1 HN protein antibody titer produced in a subject who has not been administered a vaccine against hMPV and/or PIV3 and/or RSV and/or PIV1.
E205. The immunogenic composition of embodiment E198-E204, wherein the effective amount is a total dose of between about 15 μg-200 μg.
E206. The immunogenic composition of embodiment E205, wherein the effective amount is a total dose of between about 25 μg-100 μg.
E207. A mutant of a wild-type PIV1 HN polypeptide comprising amino acids encoded by the RNA molecule of any one of embodiments E63-E73, E94 or E95.
E208. A mutant of a wild-type parainfluenza virus type 1 (PIV1) HN polypeptide, wherein the mutant comprises at least one amino acid mutation relative to the amino acid sequence of the wild type PIV1 HN polypeptide, wherein the mutation is selected from the group consisting of: i) a deletion of residues at positions 57-84; and ii) a deletion of residues at positions 57-129, wherein the amino acid positions are numbered according to SEQ ID NO: 750.
E209. The mutant PIV1 HN polypeptide according to embodiment E208, comprising the amino acid sequence set forth in any one of SEQ ID NO: 756 or 757.
E210. The mutant PIV1 HN polypeptide of embodiment E209, wherein the mutant PIV1 HN polypeptide comprises an amino acid sequence having at least 80% identity to the amino acid sequence set forth in any one of SEQ ID NO: 756 or 757.
E211. The mutant PIV1 HN polypeptide of embodiment E210, wherein the PIV1 HN polypeptide has at least 90%, 95, 96%, 97%, 98% or 99% identity to the amino acid sequence set forth in any one of SEQ ID NO: 756 or 757.
E212. The mutant PIV1 HN polypeptide according to any of embodiments E207-E211, wherein the polypeptide is isolated.
E213. A pharmaceutical composition comprising (i) the mutant PIV1 HN polypeptide according to any one of embodiments E207-E212 and (ii) a pharmaceutically acceptable carrier.
E214. An immunogenic composition comprising the mutant PIV1 HN polypeptide according to any one of embodiments E207-E212.
E215. A mutant of a wild-type PIV3 HN polypeptide comprising amino acids encoded by the RNA molecule of any one of embodiments E37-E47, E90 or E91.
E216. A mutant of a wild-type parainfluenza virus type 3 (PIV3) HN polypeptide, wherein the mutant comprises at least one amino acid mutation relative to the amino acid sequence of the wild type PIV3 HN polypeptide, wherein the mutation is selected from the group consisting of: i) a deletion of residues at positions 59-88; and ii) a deletion of residues at positions 59-130, wherein the amino acid positions are numbered according to SEQ ID NO: 724.
E217. The mutant PIV3 HN polypeptide according to embodiment E216, comprising the amino acid sequence set forth in any one of SEQ ID NO: 725 or 726.
E218. The mutant PIV3 HN polypeptide of embodiment E217, wherein the mutant PIV3 HN polypeptide comprises an amino acid sequence having at least 80% identity to the amino acid sequence set forth in any one of SEQ ID NO: 725 or 726.
E219. The mutant PIV3 HN polypeptide of embodiment E218, wherein the PIV3 HN polypeptide has at least 90%, 95, 96%, 97%, 98% or 99% identity to the amino acid sequence set forth in any one of SEQ ID NO: 725 or 726.
E220. The mutant PIV3 HN polypeptide according to any of embodiments E215-E219, wherein the polypeptide is isolated.
E221. A pharmaceutical composition comprising (i) the mutant PIV3 HN polypeptide according to any one of embodiments E215-E220 and (ii) a pharmaceutically acceptable carrier.
E222. An immunogenic composition comprising the mutant PIV3 HN polypeptide according to any one of embodiments E215-E221.
E223. A method of inducing an immune response against RSV, hMPV and/or PIV3 and/or PIV1 in a subject, comprising administering to the subject an effective amount of the RNA molecule of any one of embodiments 1-105 or the immunogenic composition of any one of embodiments E106-E205, E213 or E221.
E224. A method of preventing, treating or ameliorating an infection, disease or condition associated with RSV, hMPV and/or PIV3 and/or PIV1 in a subject, comprising administering to a subject an effective amount of the RNA molecule of any one of embodiments E1-E105 or the immunogenic composition of any one of embodiments E106-E205, E213 or E221.
E225. The method of embodiment E224, wherein the infection, disease or condition is RSV, hMPV and/or PIV3 and/or PIV1 infection-induced acute respiratory tract illness, lower respiratory tract illness, or lower respiratory tract disease, including pneumonia and bronchiolitis.
E226. The method of any one of embodiments E223-E225, wherein the subject is less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older.
E227. The method of embodiment E226, wherein the subject is immunocompromised.
E228. The method of embodiment any one of embodiments E223-E225, wherein the subject is pregnant for protection of the pregnant subject and/or the infant born to the vaccinated pregnant subject.
E229. The method of any one of embodiments E223-E228, wherein the RNA molecule or immunogenic composition is administered as a vaccine.
E230. The method of any one of embodiments E223-E229, wherein the subject is administered a single dose, two doses, three doses, or more, and optionally, a booster dose of the RNA molecule, immunogenic composition or vaccine.
E231. The immunogenic composition of any one of embodiments E106-E205, E213 or E221 for use as a component of a medicament.
E232. Use of the immunogenic composition of any one of embodiments E106-E205, E213 or E221 for the manufacture of a medicament.
E233. The immunogenic composition of any one of embodiments E106-E205, E213 or E221 for use as a medicament.
E234. The immunogenic composition of any one of embodiments E106-E205, E213 or E221 for use as a vaccine.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

### I. Examples of Definitions

Unless otherwise defined herein, scientific and technical terms used in connection with the present invention have the meanings that are commonly understood by those of ordinary skill in the art.

Throughout this application, the terms “about” and “approximately” and “substantially” are used according to their plain and ordinary meaning in the area of cell and molecular biology to indicate a deviation of +10% of the value(s) to which it is attached. Therefore, in any disclosed aspect, the terms may be substituted with “within [a percentage] of” what is specified. In one non-limiting aspect, the percentage includes 0.1, 0.5, 1, 5, and 10 percent.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one,” but it is also consistent with the meaning of “one or more,” “at least one,” and “one or more than one.”

The phrase “and/or” means “and” or “or.” To illustrate, A, B, and/or C includes: A alone, B alone, C alone, a combination of A and B, a combination of A and C, a combination of B and C, or a combination of A, B, and C. In other words, “and/or” operates as an inclusive “or”.

The phrase “essentially all” is defined as “at least 95%”; if essentially all members of a group have a certain property, then at least 95% of members of the group have that property. In some aspects, essentially all means equal to any one of, at least any one of, or between any two of 95, 96, 97, 98, 99, or 100% of members of the group have that property.

The compositions and methods for their use may “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that aspects described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “inhibiting,” “decreasing,” or “reducing” or any variation of these terms includes any measurable decrease (e.g., a 5%, 10%, 15%, 20%, 25%, 30%, 35%, 40%, 45%, 50%, 55%, 60%, 65%, 70%, 75%, 80%, 85%, 90%, 95%, or 99% decrease) or complete inhibition to achieve a desired result. The terms “improve,” “promote,” or “increase” or any variation of these terms includes any measurable increase (e.g., a 5%, 10%, 15%, 20%, 25%, 30%, 35%, 40%, 45%, 50%, 55%, 60%, 65%, 70%, 75%, 80%, 85%, 90%, 95%, or 99% increase) to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “isolated” may refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that may be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state and/or that is altered or removed from the natural state through human intervention. For example, a DNA naturally present in a living animal is not “isolated,” but a synthetic DNA, or a DNA partially or completely separated from the coexisting materials of its natural state is “isolated.” An isolated nucleic acid may exist in substantially purified form, or may exist in a non-native environment such as, for example, a cell into which the nucleic acid has been delivered.

A “nucleic acid,” as used herein, is a molecule comprising nucleic acid components and refers to DNA or RNA molecules. It may be used interchangeably with the term “polynucleotide.” A nucleic acid molecule is a polymer comprising or consisting of nucleotide monomers, which are covalently linked to each other by phosphodiester-bonds of a sugar/phosphate-backbone. Nucleic acids may also encompass modified nucleic acid molecules, such as base-modified, sugar-modified or backbone-modified etc. DNA or RNA molecules. Nucleic acids may exist in a variety of forms such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, saRNA, modRNA and complementary sequences of the foregoing described herein. Nucleic acids may encode an epitope to which antibodies may bind.

The term “epitope” refers to a moiety that is specifically recognized by an immunoglobulin (e.g., antibody or receptor) binding component. In some aspects, an epitope is comprised of a plurality of chemical atoms or groups on an antigen. In some aspects, such chemical atoms or groups are surface-exposed when the antigen adopts a relevant three-dimensional conformation. In some aspects, such chemical atoms or groups are physically near to each other in space when the antigen adopts such a conformation. In some aspects, at least some such chemical atoms are groups are physically separated from one another when the antigen adopts an alternative conformation (e.g., is linearized).

Nucleic acids may be single-stranded or double-stranded and may comprise RNA and/or DNA nucleotides and artificial variants thereof (e.g., peptide nucleic acids). In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

The term “polynucleotide” refers to a nucleic acid molecule that may be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA, or synthetic), analogs thereof, or a combination thereof. Additional coding or non-coding sequences may, but need not, be present within a polynucleotide.

In certain aspects, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some aspects, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids may be any length. They may be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or may comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

As used herein, the term “expression” of a nucleic acid sequence refers to the generation of any gene product from the nucleic acid sequence. In some aspects, a gene product may be a transcript. In some aspects, a gene product may be a polypeptide. In some aspects, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

The term “DNA,” as used herein, means a nucleic acid molecule comprising nucleotides such as deoxy-adenosine-monophosphate, deoxy-thymidine-monophosphate, deoxy-guanosine-monophosphate and deoxy-cytidine-monophosphate monomers which are composed of a sugar moiety (deoxyribose), a base moiety and a phosphate moiety, and polymerize by a characteristic backbone structure. The backbone structure is, typically, formed by phosphodiester bonds between the sugar moiety of the nucleotide, e.g., deoxyribose, of a first and a phosphate moiety of a second, adjacent monomer. The specific order of the monomers, e.g., the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing. DNA may contain all, or a majority of, deoxyribonucleotide residues. As used herein, the term “deoxyribonucleotide” means a nucleotide lacking a hydroxyl group at the 2′ position of a B-D-ribofuranosyl group. Without any limitation, DNA may encompass double stranded DNA, antisense DNA, single stranded DNA, isolated DNA, synthetic DNA, DNA that is recombinantly produced, and modified DNA.

The term “RNA,” as used herein, means a nucleic acid molecule comprising nucleotides such as adenosine-monophosphate, uridine-monophosphate, guanosine-monophosphate and cytidine-monophosphate monomers which are connected to each other along a so-called backbone. The backbone is formed by phosphodiester bonds between the sugar, e.g., ribose, of a first and a phosphate moiety of a second, adjacent monomer. RNA may be obtainable by transcription of a DNA-sequence, e.g., inside a cell. In eukaryotic cells, transcription is typically performed inside the nucleus or the mitochondria. In vivo, transcription of DNA may result in premature RNA which is processed into messenger-RNA (mRNA). Processing of the premature RNA, e.g. in eukaryotic organisms, comprises various posttranscriptional modifications such as splicing, 5′ capping, polyadenylation, export from the nucleus or the mitochondria. Mature messenger RNA is processed and provides the nucleotide sequence that may be translated into an amino acid sequence of a peptide or protein. A mature mRNA may comprise a 5′ cap, a 5′ UTR, an open reading frame, a 3′ UTR and a poly-A tail sequence. RNA may contain all, or a majority of, ribonucleotide residues. As used herein, the term “ribonucleotide” means a nucleotide with a hydroxyl group at the 2′ position of a B-D-ribofuranosyl group. In one aspect, RNA may be messenger RNA (mRNA) that relates to a RNA transcript which encodes a peptide or protein. As known to those of skill in the art, mRNA generally contains a 5′ untranslated region (5′ UTR), a polypeptide coding region, and a 3′ untranslated region (3′ UTR). Without any limitation, RNA may encompass double stranded RNA, antisense RNA, single stranded RNA, isolated RNA, synthetic RNA, RNA that is recombinantly produced, and modified RNA (modRNA).

An “isolated RNA” is defined as an RNA molecule that may be recombinant or has been isolated from total genomic nucleic acid. An isolated RNA molecule or protein may exist in substantially purified form, or may exist in a non-native environment such as, for example, a host cell.

A “modified RNA” or “modRNA” refers to an RNA molecule having at least one addition, deletion, substitution, and/or alteration of one or more nucleotides as compared to naturally occurring RNA. Such alterations may refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide may replace one or more uridine and/or cytidine nucleotides. For example, these replacements may occur for every instance of uridine and/or cytidine in the RNA sequence, or may occur for only select uridine and/or cytidine nucleotides. Such alterations to the standard nucleotides in RNA may include non-standard nucleotides, such as chemically synthesized nucleotides or deoxynucleotides. For example, at least one uridine nucleotide may be replaced with N1-methylpseudouridine in an RNA sequence. Other such altered nucleotides are known to those of skill in the art. Such altered RNA molecules are considered analogs of naturally-occurring RNA. In some aspects, the RNA is produced by in vitro transcription using a DNA template, where DNA refers to a nucleic acid that contains deoxyribonucleotides. In some aspects, the RNA may be replicon RNA (replicon), in particular self-replicating RNA, or self-amplifying RNA (saRNA).

As contemplated herein, without any limitations, RNA may be used as a therapeutic modality to treat and/or prevent a number of conditions in mammals, including humans. Methods described herein comprise administration of the RNA described herein to a mammal, such as a human. For example, in one aspect such methods of use for RNA include an antigen-coding RNA vaccine to induce robust neutralizing antibodies and accompanying/concomitant T-cell response to achieve protective immunization. In some aspects, minimal vaccine doses are administered to induce robust neutralizing antibodies and accompanying/concomitant T-cell response to achieve protective immunization. In one aspect, the RNA administered is in vitro transcribed RNA. For example, such RNA may be used to encode at least one antigen intended to generate an immune response in said mammal. Pathogenic antigens are peptide or protein antigens derived from a pathogen associated with infectious disease. In specific aspects, the pathogenic are peptide or protein antigens derived from RSV. Conditions and/or diseases that may be treated with RNA disclosed herein include, but are not limited to, those caused and/or impacted by viral infection. Such viruses include, but are not limited to, RSV.

“Prevent” or “prevention,” as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder, or condition has been delayed for a predefined period of time.

As will be understood from context, “risk” of a disease, disorder, and/or condition refers to a likelihood that a particular individual will develop the disease, disorder, and/or condition. In some aspects, risk is expressed as a percentage. In some aspects, risk is, is at least, or is at most from 0, 1, 2, 3, 4, 5, 6, 7, 8, 9, 10, 20, 30, 40, 50, 60, 70, 80, 90 up to 100%. In some aspects risk is expressed as a risk relative to a risk associated with a reference sample or group of reference samples. In some aspects, a reference sample or group of reference samples have a known risk of a disease, disorder, condition and/or event. In some aspects a reference sample or group of reference samples are from individuals comparable to a particular individual. In some aspects, risk may reflect one or more genetic attributes, e.g., which may predispose an individual toward development (or not) of a particular disease, disorder and/or condition. In some aspects, risk may reflect one or more epigenetic events or attributes and/or one or more lifestyle or environmental events or attributes. Susceptible to: An individual who is “susceptible to” a disease, disorder, and/or condition is one who has a higher risk of developing the disease, disorder, and/or condition than does a member of the general public. In some aspects, an individual who is susceptible to a disease, disorder and/or condition may not have been diagnosed with the disease, disorder, and/or condition. In some aspects, an individual who is susceptible to a disease, disorder, and/or condition may exhibit symptoms of the disease, disorder, and/or condition. In some aspects, an individual who is susceptible to a disease, disorder, and/or condition may not exhibit symptoms of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

The terms “protein,” “polypeptide,” or “peptide” are used herein as synonyms and refer to a polymer of amino acid monomers, e.g., a molecule comprising at least two amino acid residues. Polypeptides may include gene products, naturally occurring polypeptides, synthetic polypeptides, homologs, orthologs, paralogs, fragments and other equivalents, variants, and analogs of the foregoing. Polypeptides may be a single molecule or may be a multi-molecular complex such as a dimer, trimer or tetramer. A protein comprises one or more peptides or polypeptides, and may be folded into a 3-dimensional form, which may be required for the protein to exert its biological function.

As used herein, the term “wild type” or “WT” or “native” refers to the endogenous version of a molecule that occurs naturally in an organism. In some aspects, wild type versions of a protein or polypeptide are employed, however, in other aspects of the disclosure, a modified protein or polypeptide is employed to generate an immune response. The terms described above may be used interchangeably.

A “modified protein” or “modified polypeptide” or a “variant” refers to a protein or polypeptide whose chemical structure, particularly its amino acid sequence, is altered with respect to the wild type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild type activity or function in other respects, such as immunogenicity. Where a protein is specifically mentioned herein, it is in general a reference to a native (wild type) or recombinant (modified) protein. The protein may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., an antigen or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The term “fragment,” with reference to an amino acid sequence (peptide or protein), relates to a part of an amino acid sequence, e.g., a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90%, or at least 99% of the amino acid residues from an amino acid sequence. In the present disclosure, a fragment of a polypeptide, DNA nucleic acid or RNA nucleic acid sequence refers to a sequence having sequence identity of at least, at most, exactly, or between any two of 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 70% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 80% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 85% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 90% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 95% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 97% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 99% with a polypeptide, DNA nucleic acid or RNA nucleic acid sequence, from which it is derived.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some aspects, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some aspects, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some aspects, a variant polypeptide or nucleic acid shows an overall sequence identity with a reference polypeptide or nucleic acid that is at least, at most, exactly, or between any two of 85%, 86%, 87%, 88%, 89%, 90%, 91%, 92%, 93%, 94%, 95%, 96%, 97%, or 99%. In some aspects, a variant polypeptide or nucleic acid does not share at least one characteristic sequence element with a reference polypeptide or nucleic acid. In some aspects, a reference polypeptide or nucleic acid has one or more biological activities. In some aspects, a variant polypeptide or nucleic acid shares one or more of the biological activities of the reference polypeptide or nucleic acid. In some aspects, a variant polypeptide or nucleic acid lacks one or more of the biological activities of the reference polypeptide or nucleic acid. In some aspects, a variant polypeptide or nucleic acid shows a reduced level of one or more biological activities as compared to the reference polypeptide or nucleic acid. In some aspects, a polypeptide or nucleic acid of interest is considered to be a “variant” of a reference polypeptide or nucleic acid if it has an amino acid or nucleotide sequence that is identical to that of the reference but for a small number of sequence alterations at particular positions. Preferably, the variant polypeptide or nucleic acid sequence has at least one modification compared to the reference polypeptide or nucleic acid sequence, e.g., from 1 to about 20 modifications. In one aspect, the variant polypeptide or nucleic acid sequence has from 1 to about 10 modifications compared to the reference polypeptide or nucleic acid sequence. In one aspect, the variant polypeptide or nucleic acid sequence has from 1 to about 5 modifications compared to the reference polypeptide or nucleic acid sequence. In one aspect, the variant polypeptide or nucleic acid sequence has from 1 to about 4 modifications compared to the reference polypeptide or nucleic acid sequence. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. Often, a variant polypeptide or nucleic acid comprises a very small number (e.g., fewer than about 5, about 4, about 3, about 2, or about 1) number of substituted, inserted, or deleted, functional residues (e.g., residues that participate in a particular biological activity) relative to the reference. In some aspects, a variant polypeptide or nucleic acid comprises about 10, about 9, about 8, about 7, about 6, about 5, about 4, about 3, about 2, or about 1 substituted residues as compared to a reference. In some aspects, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some aspects, a variant polypeptide or nucleic acid comprises not more than about 5, about 4, about 3, about 2, or about 1 addition or deletion, and, in some aspects, comprises no additions or deletions, as compared to the reference.

In some aspects, a reference polypeptide or nucleic acid is a “wild type” or “WT” or “native” sequence found in nature, including allelic variations. A wild type polypeptide or nucleic acid sequence has a sequence that has not been intentionally modified. For the purposes of the present disclosure, “variants” of an amino acid sequence (peptide, protein, or polypeptide) comprise amino acid insertion variants, amino acid addition variants, amino acid deletion variants and/or amino acid substitution variants. “Variants” of a nucleotide sequence comprise nucleotide insertion variants, nucleotide addition variants, nucleotide deletion variants and/or nucleotide substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid or nucleic acid sequence.

Changes may be introduced by mutation into a nucleic acid, thereby leading to changes in the amino acid sequence of a polypeptide (e.g., an antigen or antibody or antibody derivative) that it encodes. Mutations may be introduced using any technique known in the art. In one aspect, one or more particular amino acid residues are changed using, for example, a site-directed mutagenesis protocol. In another aspect, one or more randomly selected residues are changed using, for example, a random mutagenesis protocol. In some aspects, however it is made, a mutant polypeptide may be expressed and screened for a desired property.

Mutations may be introduced into a nucleic acid without significantly altering the biological activity of a polypeptide that it encodes. For example, one may make nucleotide substitutions leading to amino acid substitutions at non-essential amino acid residues. Alternatively, one or more mutations may be introduced into a nucleic acid that selectively changes the biological activity of a polypeptide that it encodes. For example, the mutation may quantitatively or qualitatively change the biological activity. Examples of quantitative changes include increasing, reducing or eliminating the activity. Examples of qualitative changes include altering the antigen specificity of an antibody.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group). In some aspects, percent identity of two sequences is determined using the BLASTN or BLASTP algorithm, as available on the United States National Center for Biotechnology Information (NCBI) website.

Percentage identity is obtained by determining the number of identical positions at which the sequences to be compared correspond, dividing this number by the number of positions compared (e.g., the number of positions in the reference sequence) and multiplying this result by 100.

In some aspects, the degree of similarity or identity is given for a region that is at least, at most, exactly, or between any two of about 50%, about 60%, about 70%, about 80%, about 90%, or about 100% of the entire length of the reference sequence. For example, if the reference nucleic acid sequence consists of 200 nucleotides, the degree of identity is given for at least, at most, exactly, or between any two of about 100, about 120, about 140, about 160, about 180, or about 200 nucleotides, in some aspects, continuous nucleotides. In some aspects, the degree of similarity or identity is given for the entire length of the reference sequence.

Homologous amino acid sequences may exhibit at least, at most, exactly, or between any two of 40%, 50%, 60%, 70%, 80%, 90%, 95%, 98%, or 99% identity of the amino acid residues. In one aspect, homologous amino acid sequences exhibit at least 95% identity of the amino acid residues. In one aspect, homologous amino acid sequences exhibit at least 98% identity of the amino acid residues. In one aspect, homologous amino acid sequences exhibit at least 99% identity of the amino acid residues.

A fragment or variant of an amino acid sequence (peptide or protein) may be a “functional fragment” or “functional variant.” The term “functional fragment” or “functional variant” of an amino acid sequence relates to any fragment or variant exhibiting one or more functional properties identical or similar to those of the amino acid sequence from which it is derived, e.g., it is functionally equivalent. With respect to antigens or antigenic sequences, one particular function is one or more immunogenic activities displayed by the amino acid sequence from which the fragment or variant is derived. The term “functional fragment” or “functional variant,” as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one aspect, the modifications in the amino acid sequence of the parent molecule or sequence do not significantly affect or alter the characteristics of the molecule or sequence. The term “mutant” of a wild-type RSV F protein, “mutant” of a RSV F protein, “RSV F protein mutant,” or “modified RSV F protein” refers to a polypeptide that displays introduced mutations relative to a wild-type F protein and is immunogenic against the wild-type F protein.

An amino acid sequence (peptide, protein, or polypeptide) “derived from” a designated amino acid sequence (peptide, protein, or polypeptide) refers to the origin of the first amino acid sequence. Preferably, the amino acid sequence which is derived from a particular amino acid sequence has an amino acid sequence that is identical, essentially identical, or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In the present disclosure, a vector refers to a nucleic acid molecule, such as an artificial nucleic acid molecule. A vector may be used to incorporate a nucleic acid sequence, such as a nucleic acid sequence comprising an open reading frame. Vectors include, but are not limited to, storage vectors, expression vectors, cloning vectors, transfer vectors. A vector may be an RNA vector or a DNA vector. In some aspects the vector is a DNA molecule. In some aspects, the vector is a plasmid vector. In some aspects, the vector is a viral vector. Typically, an expression vector will contain a desired coding sequence and appropriate other sequences necessary for the expression of the operably linked coding sequence in a particular host organism (e.g., bacteria, yeast, plant, insect, or mammal) or in in vitro expression systems. Cloning vectors are generally used to engineer and amplify a certain desired fragment (typically a DNA fragment), and may lack functional sequences needed for expression of the desired fragment(s).

As used herein, the term “pharmaceutical composition” refers to an active agent, formulated together with one or more pharmaceutically acceptable carriers. Pharmaceutical compositions may be immunogenic compositions. In some aspects, active agent is present in unit dose amount appropriate for administration in a therapeutic regimen that shows a statistically significant probability of achieving a predetermined therapeutic effect when administered to a relevant population. In some aspects, pharmaceutical compositions may be specially formulated for parenteral administration, for example, by subcutaneous, intramuscular, intravenous or epidural injection as, for example, a sterile solution or suspension, or sustained-release formulation.

As used herein, the term “vaccination” refers to the administration of an immunogenic composition intended to generate an immune response, for example to a disease-associated (e.g., disease-causing) agent (e.g., a virus). In some aspects, vaccination may be administered before, during, and/or after exposure to a disease-associated agent, and in certain aspects, before, during, and/or shortly after exposure to the agent. In some aspects, vaccination includes multiple administrations, appropriately spaced in time, of a vaccine composition. In some aspects, vaccination generates an immune response to an infectious agent. In some aspects, vaccination generates an immune response to a tumor; in some such aspects, vaccination is “personalized” in that it is partly or wholly directed to epitope(s) (e.g., which may be or include one or more neoepitopes) determined to be present in a particular individual's tumors.

An immune response refers to a humoral response, a cellular response, or both a humoral and cellular response in an organism. An immune response may be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines.

As used herein, the term “combination therapy” refers to those situations in which a subject is simultaneously exposed to two or more therapeutic regimens (e.g., two or more therapeutic agents). In some aspects, the two or more regimens may be administered simultaneously; in some aspects, such regimens may be administered sequentially (e.g., all “doses” of a first regimen are administered prior to administration of any doses of a second regimen); in some aspects, such agents are administered in overlapping dosing regimens. In some aspects, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some aspects, two or more agents, or active moieties thereof, may be administered together in a combination composition, or even in a combination compound (e.g., as part of a single chemical complex or covalent entity).

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

The term “hMPV-2 mAb” refers to an hMPV A F protein prefusion specific antibody which has a heavy chain variable domain comprising an amino acid sequence of SEQ ID NO:360 and a light chain variable domain comprising an amino acid sequence of SEQ ID NO:361 as set forth in PCT Publication No. WO2024/154048, which is hereby incorporated by reference herein in its entirety.

The term “MPE8” refers to an antibody described in Corti et al. [Corti, D., Bianchi, S., Vanzetta, F., Minola, A., Perez, L., Agatic, G., Lanzavecchia, A. Cross-neutralization of four paramyxoviruses by a human monoclonal antibody. Nature, 501 (7467), 439-443 (2013)], which has a heavy chain variable domain comprising an amino acid sequence of SEQ ID NO:358 and a light chain variable domain comprising an amino acid sequence of SEQ ID NO:359 as set forth in PCT Publication No. WO2024/154048, which is hereby incorporated by reference herein in its entirety.

The term “PIA174 mAb” refers to a PIV3 F protein prefusion specific antibody described in PCT Publication No. WO2024/154048, which is hereby incorporated by reference herein in its entirety, which has a heavy chain variable domain comprising an amino acid sequence of SEQ ID NO: 364 and a light chain variable domain comprising an amino acid sequence of SEQ ID NO: 365. The amino acid sequence of SEQ ID NO:364 comprises the heavy chain variable domains and constant domains of PIA174 mAb and the amino acid sequence of SEQ ID NO: 365 comprises the light chain variable domains and constant domains of PIA174 mAb. The heavy chain variable domain of PIA174 mAb has the amino acid sequence of SEQ ID NO:553. The light chain variable domain of PIA174 mAb has the amino acid sequence of SEQ ID NO:554.

The term “PIV1-8 mAb” (also referred to as hPIV1-8 mAb) refers to a PIV1 F protein prefusion specific antibody which has a heavy chain variable domain comprising an amino acid sequence of SEQ ID NO:362 and a light chain variable domain comprising an amino acid sequence of SEQ ID NO:363 as set forth in PCT Publication No. WO2024/154048, which is hereby incorporated by reference herein in its entirety.

The term “prefusion-specific antibody” refers to an antibody that specifically binds to the F glycoprotein in a prefusion conformation, but does not bind to the F protein in a post-fusion conformation. Exemplary prefusion-specific antibodies include the MPE8, hMPV-2, PIA174, PIV1-8, D25, AM22, and AM14 antibody.

The term “prefusion conformation” or “prefusion F protein” refers to a structural conformation adopted by an F protein or mutant that can be specifically bound by a prefusion-specific antibody such as, for example, MPE8 mAb for hMPV A, hMPV-2 mAb for hMPV B, PIV1-8 mAb for PIV1, PIA174 mAb for PIV3 and D25, AM22, or AM14 for RSV.

The term “post-fusion conformation” or “post-fusion F protein” refers to a structural conformation adopted by an F protein or mutant that is not specifically bound by a prefusion-specific antibody such as, for example, MPE8 mAb for hMPV A, hMPV-2 mAb for hMPV B, PIV1-8 mAb for PIV1 and PIA174 mAb for PIV3 and D25, AM22, or AM14 for RSV. Native F protein adopts the post-fusion conformation subsequent to the fusion of the virus envelope with the host cellular membrane. F protein may also assume the post-fusion conformation outside the context of a fusion event, for example, under stress conditions such as heat and low osmolality, when extracted from a membrane, when expressed as an ectodomain, or upon storage.

The conformation (prefusion or post-fusion) of an hMPV, PIV1 or PIV3 F protein can be easily determined for example by using prefusion and/or post-fusion specific monoclonal antibodies as disclosed in detail in the examples of PCT Publication No. WO2024/154048, which is hereby incorporated by reference herein in its entirety.

Those skilled in the art will appreciate that the term “dosing regimen” may be used to refer to a set of unit doses (typically more than one) that are administered individually to a subject, typically separated by periods of time. In some aspects, a given therapeutic agent has a recommended dosing regimen, which may involve one or more doses. In some aspects, a dosing regimen comprises a plurality of doses each of which is separated in time from other doses. In some aspects, individual doses are separated from one another by a time period of the same length; in some aspects, a dosing regimen comprises a plurality of doses and at least two different time periods separating individual doses. In some aspects, all doses within a dosing regimen are of the same unit dose amount. In some aspects, different doses within a dosing regimen are of different amounts. In some aspects, a dosing regimen comprises a first dose in a first dose amount, followed by one or more additional doses in a second dose amount different from the first dose amount. In some aspects, a dosing regimen comprises a first dose in a first dose amount, followed by one or more additional doses in a second dose amount same as the first dose amount. In some aspects, a dosing regimen is correlated with a desired or beneficial outcome when administered across a relevant population (e.g., is a therapeutic dosing regimen).

### II. Gene of Interest (GOI)

The RNA molecules described herein may include a gene of interest. The gene of interest encodes a polypeptide of interest. Non-limiting examples of polypeptides of interest include, e.g., biologics, antibodies, vaccines, therapeutic polypeptides or peptides, cell penetrating peptides, secreted polypeptides, plasma membrane polypeptides, cytoplasmic or cytoskeletal polypeptides, intracellular membrane bound polypeptides, nuclear polypeptides, polypeptides associated with human disease, targeting moieties, those polypeptides encoded by the human genome for which no therapeutic indication has been identified but which nonetheless have utility in areas of research and discovery, or combinations thereof. In some aspects, 1, 2, 3, 4, 5, or more of the foregoing polypeptides of interest may be excluded. The sequence for a particular gene of interest is readily identified by one of skill in the art using public and private databases, e.g., GENBANK®.

### A. Respiratory Syncytial Virus (RSV)

The present disclosure provides for RNA molecules (e.g., RNA polynucleotides) comprising at least one open reading frame encoding a respiratory syncytial virus (RSV) polypeptide. The present disclosure further provides for an immunogenic composition comprising at least one RNA molecule encoding an RSV polypeptide complexed with, encapsulated in, or formulated with one or more lipids, and forming lipid nanoparticles (LNPs). The RSV polypeptide to be included in the immunogenic composition disclosed herein can be any RSV F protein in the prefusion conformation.

The term “prefusion conformation” refers to a structural conformation adopted by an RSV F protein or mutant thereof that can be specifically bound by (i) antibody D25 or AM22 when the RSV F protein or mutant is in the form of a monomer or trimer, or (ii) by antibody AM14 when the RSV F protein mutant is in the form of a trimer. The prefusion trimer conformation is a subset of prefusion conformations. As used herein, an RSV F protein or polypeptide or mutant thereof in prefusion conformation may be denoted as “RSV preF” or “RSV F”.

The term “post-fusion conformation” refers to a structural conformation adopted by the RSV F protein that is not specifically bound by D25, AM22, or AM14. Native F protein adopts the post-fusion conformation subsequent to the fusion of the virus envelope with the host cellular membrane. RSV F protein may also assume the post-fusion conformation outside the context of a fusion event, for example, under stress conditions such as heat and low osmolality, when extracted from a membrane, when expressed as an ectodomain, or upon storage. The term “AM14” refers to an antibody described in WO 2008/147196 A2, which is hereby incorporated by reference in its entirety. The term “AM22” refers to an antibody described in WO 2011/043643 A1, which is hereby incorporated by reference in its entirety. The term “D25” refers to an antibody described in WO 2008/147196 A2, which is hereby incorporated herein by reference in its entirety.

In some embodiments, the RSV F protein is an RSV F protein of subtype A. In some embodiments, the RSV F protein is an RSV F protein of subtype B. As used herein the terms “subtype” and “subgroup” are used interchangeably. As used herein the term “strain” refers to a specific isolate within each subtype or subgroup. In some embodiments, the RSV F protein is a mutant of wild type RSV F protein. In some embodiments, the RSV F protein is a mutant of wild type RSV F protein of subtype A. In some embodiments, the RSV F protein is a mutant of wild type RSV F protein of subtype B. In some embodiments, the mutants display introduced mutations in the amino acid sequence relative to the amino acid sequence of the corresponding wild-type RSV F protein and are immunogenic against the wild-type RSV F protein in the prefusion conformation or against a virus comprising the wild-type F protein. The amino acid mutations in the mutants include amino acid substitutions, deletions, or additions relative to a wild-type RSV F protein.

In some embodiments, the RSV F protein is an RSV protein mutant as described in WO2017/109629, which is hereby incorporated herein by reference in its entirety.

In some embodiments, the RSV F protein is a mutant of a wild-type RSV F protein, wherein the introduced amino acid mutations are mutation of a pair of amino acid residues in a wild-type RSV F protein to a pair of cysteines (“engineered disulfide mutation”). The introduced pair of cysteine residues allows for formation of a disulfide bond between the cysteine residues that stabilize the protein's conformation or oligomeric state, such as the prefusion conformation. Examples of specific pairs of such mutations include: 55C and 188C; 155C and 290C; 103C and 148C; and 142C and 371C, such as S55C and L188C; S155C and S290C; A103C and I148C; and L142C and N371C.

In still other embodiments, the RSV F protein mutants comprise amino acid mutations that are one or more cavity filling mutations. Examples of amino acids that may be replaced with the goal of cavity filling include small aliphatic (e.g. Gly, Ala, and Val) or small polar amino acids (e.g. Ser and Thr) and amino acids that are buried in the prefusion conformation, but exposed to solvent in the post-fusion conformation. Examples of the replacement amino acids include large aliphatic amino acids (Ile, Leu and Met) or large aromatic amino acids (His, Phe, Tyr and Trp). In some specific embodiments, the RSV F protein mutant comprises a cavity filling mutation selected from the group consisting of:
(1) substitution of S at positions 55, 62, 155, 190, or 290 with I, Y, L, H, or M;
(2) substitution of T at position 54, 58, 189, 219, or 397 with I, Y, L, H, or M;
(3) substitution of G at position 151 with A or H;
(4) substitution of A at position 147 or 298 with I, L, H, or M;
(5) substitution of V at position 164, 187, 192, 207, 220, 296, 300, or 495 with I, Y, H; and
(6) substitution of R at position 106 with W.

In some particular embodiments, the RSV F protein mutant comprises at least one cavity filling mutation selected from the group consisting of: T54H, S190I, and V296I.

In still other embodiments, the RSV F protein mutants comprise electrostatic mutations, which decrease ionic repulsion or increase ionic attraction between resides in a protein that are proximate to each other in the folded structure. In several embodiments, the RSV F protein mutant includes an electrostatic substitution that reduces repulsive ionic interactions or increases attractive ionic interactions with acidic residues of Glu487 and Asp489 from another protomer of RSV F trimer. In some specific embodiments, the RSV F protein mutant comprises an electrostatic mutation selected from the group consisting of:
(1) substitution of E at position 82, 92, or 487 by D, F, Q, T, S, L, or H;
(2) substitution of K at position 315, 394, or 399 by F, M, R, S, L, I, Q, or T;
(3) substitution of D at position 392, 486, or 489 by H, S, N, T, or P; and
(4) substitution of R at position 106 or 339 by F, Q, N, or W.

In still other embodiments, the RSV F protein mutants comprise a combination of two or more different types of mutations selected from engineered disulfide mutations, cavity filling mutations, and electrostatic mutations. In some particular embodiments, the RSV F protein mutants comprise a combination of mutations relative to the corresponding wild-type RSV F protein, wherein the combination of mutations is selected from the group consisting of:
(1) combination of A103C, I148C, S190I, and D486S;
(2) combination of T54H S55C L188C D486S;
(3) combination of T54H, A103C, I148C, S190I, V296I, and D486S;
(4) combination of T54H, S55C, L142C, L188C, V296I, and N371C;
(5) combination of S55C, L188C, and D486S;
(6) combination of T54H, S55C, L188C, and S190I;
(7) combination of S55C, L188C, S190I, and D486S;
(8) combination of T54H, S55C, L188C, S190I, and D486S;
(9) combination of S155C, S190I, S290C, and D486S;
(10) combination of T54H, S55C, L142C, L188C, V296I, N371C, D486S, E487Q, and D489S;
(11) combination of T54H, S155C, S190I, S290C, and V296I, and,
(12) combination of S155C, S190F, S290C, and V207L.

In some embodiments, the RSV F protein is of subtype A and comprises the mutations S155C, S190F, S290C, and V207L.

In some embodiments, the RSV F protein is of subtype B and comprises the mutations S155C, S190F, S290C, and V207L.

In some embodiments, the RSV F protein is of subtype A and comprises the mutations S155C, S190F, and S290C.

In some embodiments, the RSV F protein is of subtype B and comprises the mutations S155C, S190F, and S290C.

In some embodiments, the RSV F protein is of subtype A and comprises the mutations A103C, I148C, S190I, and D486S.

In some embodiments, the RSV F protein is of subtype B and comprises the mutations A103C, I148C, S190I, and D486S.

In some embodiments, the RSV F protein is of subtype A and comprises the mutations T54H, A103C, I148C, S190I, and D486S.

In some embodiments, the RSV F protein is of subtype B and comprises the mutations T54H, A103C, I148C, S190I, and D486S.

In some embodiments, the RSV F protein is of subtype A and comprises the mutations T54H, S55C, L188C, and D486S.

In some embodiments, the RSV F protein is of subtype B and comprises the mutations T54H, S55C, L188C, and D486S.

In view of the substantial conservation of RSV F sequences, a person of ordinary skill in the art can easily compare amino acid positions between different native RSV F sequences to identify corresponding RSV F amino acid positions between different RSV strains and subtypes. For example, across nearly all identified native RSV F0 precursor proteins, the furin cleavage sites fall in the same amino acid positions. Thus, the conservation of native RSV F protein sequences across strains and subtypes allows use of a reference RSV F sequence for comparison of amino acids at particular positions in the RSV F protein. For the purposes of this disclosure (unless context indicates otherwise), the RSV F protein amino acid positions are given with reference to the amino acid sequence of the full length native F precursor polypeptide of the RSV A2 strain; corresponding to GenInfo Identifier GI 138251 and Swiss Prot identifier P03420 (SEQ ID NO: 1).

In some embodiments, the RSV F protein is in the mature form of the RSV F protein, which comprises two separate polypeptide chains, namely the F1 polypeptide and F2 polypeptide. In some other embodiments, the F2 polypeptide is linked to the F1 polypeptide by one or two disulfide bonds to form a F2/F1 heterodimer. In still other embodiments, the RSV F mutants are in the form a single chain protein, wherein the F2 polypeptide is linked to the F1 polypeptide by a peptide bond or peptide linker. Any suitable peptide linkers for joining two polypeptide chains together may be used. Examples of such linkers include G, GG, GGG, GS, and SAIG linker sequences. The linker may also be the full length pep27 sequence or a fragment thereof, which full length pep27 sequence corresponds to amino acids at positions 110-136 of SEQ ID NO:1.

The F1 polypeptide chain of the mutant may be of the same length as the full length F1 polypeptide of the corresponding wild-type RSV F protein; however, it may also have deletions, such as deletions of 1 up to 60 amino acid residues from the C-terminus of the full-length F1 polypeptide. A full-length F1 polypeptide of the RSV F mutants corresponds to amino acid positions 137-574 of the native RSV F0 precursor (SEQ ID NO: 1), and includes (from N- to C-terminus) an extracellular region (residues 137-524), a transmembrane domain (“TM”) (residues 525-550), and a cytoplasmic domain (“CT”) (residues 551-574). It should be noted that amino acid residues 514 onwards in a native F1 polypeptide sequence are optional sequences in a F1 polypeptide of the RSV F protein to be included in the immunogenic composition provided herein, and therefore may be absent from the F1 polypeptide of the mutant.

In some embodiments, the F1 polypeptide of the RSV F mutants lacks the entire cytoplasmic domain. In other embodiments, the F1 polypeptide lacks the cytoplasmic domain and a portion of or all entire transmembrane domain. In some specific embodiments, the mutant comprises a F1 polypeptide wherein the amino acid residues from position 510, 511, 512, 513, 514, 515, 520, 525, or 530 through 574 are absent. Typically, for mutants that are linked to trimerization domain, such as a foldon, amino acids 514 through 574 can be absent. Thus, in some specific embodiment, amino acid residues 514 through 574 are absent from the F1 polypeptide of the mutant. In still other specific embodiments, the F1 polypeptide of the RSV F mutants comprises or consists of amino acid residues 137-513 of a native F0 polypeptide sequence (SEQ ID NO: 1) or any of alternative F0 precursor sequence such as those disclosed in SEQ ID NOs: 1, 2, 4, 6, and 81-270 of WO2017109629, which is hereby incorporated by reference in its entirety.

The F1 polypeptide and F2 polypeptide of the RSV F protein mutants to which one or more mutations are introduced can be from any wild-type RSV F proteins known in the art or discovered in the future, including, without limitations, the F protein amino acid sequence of RSV subtype A, and subtype B strains, including A2 Ontario and Buenos Aires, or any other subtype. In some embodiments, the RSV F mutant comprises a F1 and/or a F2 polypeptide from a RSV A virus, for example, a F1 and/or F2 polypeptide from a RSV F0 precursor protein set forth in any one of SEQ ID NOs: 1, 2, 4, 6, and 81-270 of WO2017109629, which sequences are hereby incorporated by reference in their entireties, to which one or more mutations are introduced. In some other embodiments, the RSV F mutant comprises a F1 and/or a F2 polypeptide from a RSV B virus, for example, a F1 and/or F2 polypeptide from a RSV F0 precursor protein set forth in any one of SEQ ID NOs: 2 and 211-263 of WO2017/109629, which sequences are hereby incorporated by reference in their entireties, to which one or more mutations are introduced. In still other embodiments, the RSV F mutant comprises a F1 and/or a F2 polypeptide from a RSV bovine virus, for example, a F1 and/or F2 polypeptide from a RSV F0 precursor protein set forth in any one of SEQ ID NOs: 264-270 of WO2017109629, which sequences are hereby incorporated by reference in their entireties, to which one or more mutations are introduced.

The term “F0 polypeptide” (F0) of RSV refers to the precursor polypeptide of the RSV F protein, which is composed of a signal polypeptide sequence, a F1 polypeptide sequence, a pep27 polypeptide sequence, and a F2 polypeptide sequence. With rare exceptions the F0 polypeptides of the known RSV strains consist of 574 amino acids.

The term “F1 polypeptide” (F1) of RSV refers to a polypeptide chain of a mature RSV F protein. Native F1 includes approximately residues 137-574 of the RSV F0 precursor and is composed of (from N- to C-terminus) an extracellular region (approximately residues 137-524), a transmembrane domain (“TM”) (approximately residues 525-550), and a cytoplasmic tail (“CT”) (approximately residues 551-574). As used herein, the term encompasses both native F1 polypeptides and F1 polypeptides including modifications (e.g., amino acid substitutions, insertions, or deletions) from the native sequence, for example, modifications designed to stabilize an RSV F protein mutant or to enhance the immunogenicity of an RSV F protein mutant.

The term “F2 polypeptide” (F2) refers to the polypeptide chain of a mature RSV F protein. Native F2 includes approximately residues 26-109 of the RSV F0 precursor. As used herein, the term encompasses both native F2 polypeptides and F2 polypeptides including modifications (e.g., amino acid substitutions, insertions, or deletions) from the native sequence, for example, modifications designed to stabilize an RSV F protein mutant in a prefusion conformation or to enhance the immunogenicity of an RSV F protein mutant. In native RSV F protein, the F2 polypeptide is linked to the F1 polypeptide by two disulfide bonds to form a F2-F1 heterodimer. The term “foldon” or “foldon domain” refers to an amino acid sequence that is capable of forming trimers. One example of such foldon domains is the peptide sequence derived from bacteriophage T4 fibritin, which has the sequence of GYIPEAPRDGQAYVRKDGEWVLLSTFL (SEQ ID NO: 45).

In some aspects, the RNA molecule encodes an RSV F protein mutant as disclosed in WO2009/079796, WO2010/149745, WO2011/008974, WO2014/160463, WO2014/174018, WO2014/202570, WO2015/013551, WO2015/177312, WO2017/005848, WO2017/174564, WO2017/005844 and WO2018/109220. The RSV F proteins disclosed in these references are hereby incorporated by reference in their entirety.

Antibodies to RSV F protein are prevalent after natural infection and following vaccination and have been shown to neutralize viral activity in vitro. As used herein, the term “respiratory syncytial virus” or “RSV” is not limited to any particular strain or variant.

In some aspects, the RNA molecule comprises an open reading frame encoding a RSV antigen. In some aspects, the RSV antigen is a RSV polypeptide. In some aspects, the RSV polypeptide is a RSV glycoprotein or a fragment or a variant thereof. In some aspects, the RNA molecule encodes a RSV F protein.

In some aspects, the RSV polypeptide is a full-length RSV polypeptide. In some aspects, the RSV polypeptide is a truncated RSV polypeptide. In some aspects, the RSV polypeptide is a variant of a RSV polypeptide. In some aspects, the RSV polypeptide is a fragment of a RSV polypeptide.

In some aspects, the RSV polypeptide is a full-length RSV F protein. In some aspects, the RSV polypeptide is a truncated RSV F protein. In some aspects, the RSV polypeptide is a variant of a RSV F protein. In some aspects, the RSV polypeptide is a fragment of a RSV F protein.

In some aspects, the RSV F protein comprises at least one mutation. In some aspects, the RSV F protein comprises at least two mutations. In some aspects, the RSV F protein comprises at least three mutations. In some aspects, the RSV F protein comprises at least four mutations. In some aspects, the RSV F protein comprises 4 mutations. In some aspects, the RSV F protein comprises at least five mutations.

In some aspects, the RNA molecule encodes a RSV F protein as set forth in Table 1. In some aspects, the RNA molecule encodes a RSV F protein comprising an amino acid sequence of any of SEQ ID NO: 1 to 6, or fragment or variant thereof. In some aspects, RSV F polypeptide may have at least, at most, exactly, or between any two of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to any of the amino acid sequences of Table 1, for example, any of SEQ ID NO: 1 to 6. In some aspects, RSV F protein consists of any of the amino acid sequences of Table 1, for example, any of SEQ ID NO: 1 to 6.

In some aspects, the RNA molecule sequence is transcribed from a DNA nucleic acid sequence (DNA polynucleotide) of Table 2 and at least one untranslated region (UTR) selected from Table 21, provided that when the ORF is SEQ ID NO: 8 or 10 that the UTR does not consist of only SEQ ID NOS: 111 and 280. In some aspects, the RNA molecule comprises an ORF transcribed from a nucleic acid sequence of any of SEQ ID NO: 7 to 10, or fragment or variant thereof. In some aspects, the RNA molecule is transcribed from a nucleic acid having a sequence that may have at least, at most, exactly, or between any two of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to any of the nucleic acid sequences of Table 2, for example, any of SEQ ID NO: 7 to 14. In some aspects, the RNA molecule is transcribed from a nucleic acid having a sequence that consists of any of the nucleic acid sequences of Table 2, for example, any of SEQ ID NO:7 to 14.

In some aspects, the RNA molecule comprises an ORF encoding RSV F comprising an RNA nucleic acid sequence (RNA polynucleotide) of Table 3 and at least one untranslated region (UTR) selected from Table 22, provided that when the ORF is SEQ ID NO: 15 or 16 that the UTR does not consist of only SEQ ID NOs: 397 and 566. In some aspects, the RNA molecule comprises a nucleic acid having a sequence of any of SEQ ID NO: 15 to 20, or fragment or variant thereof. In some aspects, the RNA molecule comprises a nucleic acid having a sequence that may have at least, at most, exactly, or between any two of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to any of the RNA nucleic acid sequences of Table 3, for example, any of SEQ ID NO: 15 to 20. In some aspects, the RNA molecule comprises a nucleic acid having a sequence that consists of any of the RNA nucleic acid sequences of Table 3, for example, any of SEQ ID NO: 15 to 20.

In some aspects, the RNA molecule comprises stabilized RNA. In some aspects, the RNA molecule comprises a nucleic acid sequence having at least one uridine replaced by N1-methylpseudouridine. In some aspects, the RNA molecule comprises a sequence having all uridines replaced by N1-methylpseudouridine (designated as “m1ψ”). In some aspects, the RNA molecule comprises an ORF comprising a nucleic acid sequence of any of SEQ ID NO: 15 or 16, wherein all uridines have been replaced by N1-methylpseudouridine (designated as “m1Y”).

In some aspects, the RNA molecule comprises an open reading frame encoding a RSV F protein amino acid sequence that may be at least, at most, exactly, or between any two of 70%, 80%, 85%, 86%, 87%, 88%, 89%, 90%, 91%, 92%, 93%, 94%, 95%, 96%, 97%, 98%, or 99% identical to any of the RSV F protein sequences of SEQ ID NO: 1 to 6 (Table 1) or other RSV prefusion F proteins described herein. In some aspects, the RNA molecule comprises an open reading frame encoding a RSV F protein amino acid sequence that consists of any of the RSV F protein sequences of SEQ ID NO: 1 to 6 (Table 1) or other RSV prefusion F protein described herein.

In some aspects, the RNA molecule comprises an open reading frame transcribed from a DNA nucleic acid sequence that may be at least, at most, exactly, or between any two of 70%, 80%, 85%, 86%, 87%, 88%, 89%, 90%, 91%, 92%, 93%, 94%, 95%, 96%, 97%, 98%, or 99% identical to any of the nucleic acid sequences of SEQ ID NO: 7 to 14 (Table 2) or other nucleic acid described herein. In some aspects, the RNA molecule comprises an open reading frame transcribed from a DNA nucleic acid sequence that consists of any of the nucleic acid sequences of SEQ ID NO: 7 to 14 (Table 2) or other nucleic acid described herein.

In some aspects, the RNA molecule comprises an open reading frame encoding a RSV F, wherein the RNA nucleic acid sequence may be at least, at most, exactly, or between any two of 70%, 80%, 85%, 86%, 87%, 88%, 89%, 90%, 91%, 92%, 93%, 94%, 95%, 96%, 97%, 98%, or 99% identical to any of the nucleic acid sequences of SEQ ID NO: 15 to 20 (Table 3) or other nucleic acid described herein. In some aspects, the RNA molecule comprises an open reading frame encoding a RSV F, wherein the RNA nucleic acid sequence consists of any of the nucleic acid sequences of SEQ ID NO: 15 to 20 (Table 3) or other nucleic acid described herein. In some aspects, the RNA molecule comprises an ORF encoding a RSV F, wherein the nucleic acid sequence is any of SEQ ID NO: 15 to 20 (Table 3), wherein all uridines have been replaced by N1-methylpseudouridine (designated as “m1Y”).

B. Human Metapneumovirus (hMPV)

The present disclosure provides for RNA molecules (e.g., RNA polynucleotides) comprising at least one open reading frame encoding a human metapneumovirus (hMPV) polypeptide. The present disclosure further provides for an immunogenic composition comprising at least one RNA molecule encoding an hMPV polypeptide complexed with, encapsulated in, or formulated with one or more lipids, and forming lipid nanoparticles (LNPs). The hMPV polypeptide encoded by the RNA molecule in the immunogenic composition disclosed herein can be any hMPV F protein in the prefusion conformation. As used herein, an hMPV F protein or polypeptide or mutant thereof in prefusion conformation may be denoted as “hMPV preF” or “hMPV F”.

In some embodiments, the hMPV F protein is an hMPV F protein of subtype A. In some embodiments, the hMPV F protein is an hMPV F protein of subtype B. As used herein the terms “subtype” and “subgroup” are used interchangeably. As used herein the term “strain” refers to a specific isolate within each subtype or subgroup. In some embodiments, the hMPV F protein is a mutant of wild type hMPV F protein. In some embodiments, the hMPV F protein is a mutant of wild type hMPV F protein of subtype A. In some embodiments, the hMPV F protein is a mutant of wild type hMPV F protein of subtype B. In some embodiments, the mutants display introduced mutations in the amino acid sequence relative to the amino acid sequence of the corresponding wild-type hMPV F protein and are immunogenic against the wild-type hMPV F protein in the prefusion conformation or against a virus comprising the wild-type F protein. The amino acid mutations in the mutants include amino acid substitutions, deletions, or additions relative to a wild-type hMPV F protein.

In some embodiments, the present invention provides nucleic acid molecules that encode a hMPV F protein mutant described in PCT Pub. No. WO2024154048, which is hereby incorporated by reference herein in its entirety. These nucleic acid molecules include DNA, cDNA, and RNA sequences. Nucleic acid molecules that encode only a F2 polypeptide or only a F1 polypeptide of a hMPV F mutant are also encompassed by the invention. The nucleic acid molecule can be incorporated into a vector, such as an expression vector.

In some embodiments, the nucleic acid molecule encodes a precursor F0 polypeptide that, when expressed in an appropriate cell, is processed into a disclosed hMPV F mutant. In some embodiments, the nucleic acid molecule encodes a precursor F0 polypeptide that, when expressed in an appropriate cell, is processed into a disclosed hMPV F mutant, wherein the precursor F0 polypeptide includes, from N- to C-terminus, a signal peptide, a F2 polypeptide, and a F1 polypeptide. The term “F0 polypeptide” (F0) when used in connection with hMPV F protein, refers to the precursor polypeptide of the hMPV F protein, which is composed of a signal polypeptide sequence, a F1 polypeptide sequence and a F2 polypeptide sequence. With rare exceptions the F0 polypeptides of the known hMPV strains consist of 539 amino acids. In some embodiments, the signal peptide comprises the amino acid sequence set forth as positions 1-18 of any one SEQ ID NOs: 639-647, 715-716 and 721 wherein the amino acid positions correspond to the amino acid sequence of a reference of SEQ ID NO: 639.

In a preferred embodiment, the nucleic acid is an RNA, more preferably an mRNA. In a preferred embodiment, the mRNA encodes a precursor F0 polypeptide that, when expressed in an appropriate cell, is processed into a full length hMPV F protein mutant disclosed herein (i.e comprising one or more mutations, a full length F1 polypeptide and a full length F2 polypeptide). A full-length F1 polypeptide of the hMPV F mutants corresponds to amino acid positions 103-539 of the native hMPV F0 precursor, and includes (from N- to C-terminus) an extracellular region (residues 103 to 489), a transmembrane domain (residues 490-514), and a cytoplasmic domain (residues 515-539). In a preferred embodiment, the nucleic acid is an mRNA comprising a chemically modified nucleotide. In a preferred embodiment, the nucleic acid is an mRNA comprising a chemically modified nucleotide, preferably N1-methylpseudouridine (m1ψ). Preferably, all the uridines of the RNA are replaced by N1-methylpseudouridine.

In some embodiments, the nucleic acid molecule encodes a mutant selected from the group consisting of:
(1) a mutant comprising at least one engineered disulfide bond mutation;
(2) a mutant comprising at least one cavity filling mutation;
(3) a mutant comprising at least one proline substitution mutation;
(4) a mutant comprising at least one glycine replacement mutation;
(5) a mutant comprising a combination of at least one engineered disulfide mutation and at least one cavity filling mutation;
(6) a mutant comprising a combination of at least one engineered disulfide mutation and at least one proline substitution mutation;
(7) a mutant comprising a combination of at least one engineered disulfide mutation and a least one glycine replacement mutation;
(8) a mutant comprising a combination of at least one engineered disulfide mutation, at least one cavity filling mutation and at least one proline substitution mutation;
(9) a mutant comprising a combination of at least one engineered disulfide mutation, at least one cavity filling mutation, and a least one glycine replacement mutation;
(10) a mutant comprising a combination of at least one engineered disulfide mutation, at least one proline substitution mutation and a least one glycine replacement mutation; and, (11) a mutant comprising a combination of at least one engineered disulfide mutation, at least one cavity filling mutation, at least one proline substitution mutation and at least one glycine replacement mutation.

In some specific embodiments, the present disclosure provides a nucleic acid molecule which encodes a mutant selected from the group consisting of:
(1) a mutant comprising a combination of substitutions 140C and 149C;
(2) a mutant comprising a combination of substitutions 140C, 149C, 411C and 434C;
(3) a mutant comprising a combination of substitutions 140C, 149C, 411C, 434C and 459P;
(4) a mutant comprising a combination of substitutions 140C, 149C, 411C, 434C and 365I;
(5) a mutant comprising a combination of substitutions 140C, 149C, 411C, 434C and G239A;
(6) a mutant comprising a combination of substitutions 140C, 149C, 411C, 434C, 459P, G239A, 49I and 365I;
(7) a mutant comprising a combination of substitutions 411C, 434C, 141C and 161C;
(8) a mutant comprising a combination of substitutions 411C, 434C, 141C, 161C and 459P;
(9) a mutant comprising a combination of substitutions 411C, 434C, 141C, 161C and 49I;
(10) a mutant comprising a combination of substitutions 411C, 434C, 141C, 161C and 365I;
(11) a mutant comprising a combination of substitutions 411C, 434C, 141C, 161C and G239A;
(12) a mutant comprising a combination of substitutions 411C, 434C, 141C, 161C and 149T;
(13) a mutant comprising a combination of substitutions 411C, 434C, 141C, 161C, 459P, G239A, 49I, 149T and 365I;
(14) a mutant comprising a combination of substitutions 411C, 434C, 141C, 161C and 365I; and
(15) a mutant comprising a combination of substitutions 411C, 434C, 146C, 160C, 459P, G239A, 49I, 149T and 365I.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length hMPV F protein mutant disclosed herein comprising the mutations selected from the group of
(1) A140C and S149C,
(2) A140C, S149C, T411C and Q434C,
(3) A140C, S149C, T411C, Q434C and A459P,
(4) A140C, S149C, T411C, Q434C and T365I,
(5) A140C, S149C, T411C, Q434C and G239A,
(6) A140C, S149C, T411C, Q434C, A459P, G239A, T49I and T365I,
(7) T411C, Q434C, L141C and A161C,
(8) T411C, Q434C, L141C, A161C and A459P,
(9) T411C, Q434C, L141C, A161C and T49I,
(10) T411C, Q434C, L141C, A161C and T365I,
(11) T411C, Q434C, L141C, A161C and G239A,
(12) T411C, Q434C, L141C, A161C and S149T,
(13) T411C, Q434C, L141C, A161C, A459P, G239A, T49I, S149T and T365I, and,
(14) T411C, Q434C, E146C, T160C, A459P, G239A, T49I, S149T and T365I.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length hMPV F protein mutant disclosed comprising the mutations selected from the group consisting of
(1) T150C, R156C and A459P;
(2) T150C, R156C and T49I;
(3) T150C, R156C, T49I and A459P;
(4) A140C, S149C, T411C, and Q434C;
(5) L141C, A161C, T411C, and 434C;
(6) A140C, S149C, T411C, Q434C, and A459P;
(7) A140C, S149C, G239A, T411C and Q434C;
(8) L141C, A161C, T411C, Q434C and A459P;
(9) L141C, A161C, G239A, T411C and Q434C;
(10) T49I, T150C, R156C, G239A and A459P;
(11) A140C, S149C, G239A, T411C, Q434C and A459P;
(12) T49I, A140C, S149C, G239A, T411C, Q434C and A459P;
(13) T49I, A140C, S149C, G239A, T365I, T411C, Q434C and A459P;
(14) L141C, A161C, G239A, T411C, Q434C and A459P;
(15) T49I, L141C, A161C, G239A, T411C, Q434C and A459P;
(16) L141C, A161C, S149T, G239A, T411C, Q434C and A459P;
(17) T49I, L141C, A161C, S149T, G239A, T411C, Q434C and A459P;
(18) T49I, L141C, A161C, S149T, G239A, T365I, T411C, Q434C and A459P;
(19) T49I, S149T and A459P;
(20) A140C, S149C and A459P;
(21) T49I, A140C and S149C;
(22) T49I, A140C, S149C and A459P;
(23) T49I, L141C, A161C, T411C and Q434C;
(24) T49I, L141C, A161C, T411C, Q434C and A459P;
(25) L141C, A161C and S149T;
(26) L141C, A161C, S149T and A459P;
(27) T49I, L141C, A161C and S149T, and,
(28) T49I, L141C, A161C, S149T and A459P;
(29) V84C, del89-112 replaced with GSGGSG Linker, A140C, A147C, D185P, A249C, D454C, V458C;
(30) V84C, del89-112 replaced with GSGGSG Linker, A140C, A147C, A185P, A249C, D454C, and V458C; and
(31) Q100R, S101R, and A185P,
   wherein “del89-112” denotes deletion of amino acid residues from positions 89-112 of the mutant.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length hMPV F protein mutant disclosed herein comprising the mutations selected from the group consisting of
(1) L66P;
(2) L187P;
(4) A140C, S149C and L187P;
(5) T49I;
(6) T365I; and,
(7) T49I and T365I.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length hMPV F protein mutant disclosed herein comprising the mutations selected from the group consisting of
(1) L187P, Q100R and S101R; and,
(2) A140C, S149C, L187P, Q100R and S101R.

### C. Parainfluenza Virus Type 1 (PIV1)

The present disclosure provides for RNA molecules (e.g., RNA polynucleotides) comprising at least one open reading frame encoding a Parainfluenza Virus Type 1 (PIV1) polypeptide. The present disclosure further provides for an immunogenic composition comprising at least one RNA molecule encoding an PIV1 polypeptide complexed with, encapsulated in, or formulated with one or more lipids, and forming lipid nanoparticles (LNPs). The PIV1 polypeptide encoded by the RNA molecule in the immunogenic composition disclosed herein can be any PIV1 F protein in the prefusion conformation and/or any PIV1 HN protein.

As used herein, a PIV1 F protein or polypeptide or mutant thereof in prefusion conformation may be denoted as “PIV1 preF” or “PIV1 F”. In some embodiments, the PIV1 F protein is a mutant of wild type PIV1 F protein. In some embodiments, the mutants display introduced mutations in the amino acid sequence relative to the amino acid sequence of the corresponding wild-type PIV1 F protein and are immunogenic against the wild-type PIV1 F protein in the prefusion conformation or against a virus comprising the wild-type F protein. The amino acid mutations in the mutants include amino acid substitutions, deletions, or additions relative to a wild-type PIV1 F protein.

In some embodiments, the PIV1 HN protein is a mutant of wild type PIV1 HN protein. In some embodiments, the mutants display introduced mutations in the amino acid sequence relative to the amino acid sequence of the corresponding wild-type PIV1 HN protein and are immunogenic against the wild-type PIV1 HN protein or against a virus comprising the wild-type HN protein. The amino acid mutations in the mutants include amino acid substitutions, deletions, or additions relative to a wild-type PIV1 HN protein.

In some embodiments, the present invention provides nucleic acid molecules that encode a PIV1 F and/or HN protein mutant described herein above. These nucleic acid molecules include DNA, cDNA, and RNA sequences. Nucleic acid molecules that encode only a F2 polypeptide or only a F1 polypeptide of a PIV1 F protein mutant are also encompassed by the invention. The nucleic acid molecule can be incorporated into a vector, such as an expression vector.

In some embodiments, the nucleic acid molecule encodes a precursor F0 polypeptide that, when expressed in an appropriate cell, is processed into a disclosed PIV1 F protein mutant. In some embodiments, the nucleic acid molecule encodes a precursor F0 polypeptide that, when expressed in an appropriate cell, is processed into a disclosed PIV1 F protein mutant, wherein the precursor F0 polypeptide includes, from N- to C-terminus, a signal peptide, a F2 polypeptide, and a F1 polypeptide. In some embodiments, the signal peptide comprises the amino acid sequence set forth as positions 1-21 of any one SEQ ID NOs: 672-676, wherein the amino acid positions correspond to the amino acid sequence of reference of SEQ ID NO:672.

In a preferred embodiment, the nucleic acid is an RNA, more preferably an mRNA. In a preferred embodiment, the mRNA encodes a precursor F0 polypeptide that, when expressed in an appropriate cell, is processed into a full length PIV1 F protein mutant disclosed herein (i.e comprising one or more mutations, a full length F1 polypeptide and a full length F2 polypeptide). A full-length F1 polypeptide of the PIV1 F mutants corresponds to amino acid positions 113-555 of the native PIV1 F0 precursor, and includes (from N- to C-terminus) an extracellular region (residues 113 to 496), a transmembrane domain (residues 497-517), and a cytoplasmic domain (residues 518-555). In a preferred embodiment, the nucleic acid is an mRNA comprising a chemically modified nucleotide. In a preferred embodiment, the nucleic acid is an mRNA comprising a chemically modified nucleotide, preferably N1-methylpseudouridine. Preferably, all the uridines of the RNA are replaced by N1-methylpseudouridine.

In some embodiments, the nucleic acid molecule encodes a PIV1 F protein mutant selected from the group consisting of:
(1) a mutant comprising at least one engineered disulfide bond mutation;
(2) a mutant comprising at least one cavity filling mutation;
(3) a mutant comprising at least one proline substitution mutation;
(4) a mutant comprising at least one glycine replacement mutation;
(5) a mutant comprising at least one cleavage site mutation
(6) a mutant comprising a combination of at least one engineered disulfide mutation and at least one cavity filling mutation;
(7) a mutant comprising a combination of at least one engineered disulfide mutation and at least one proline substitution mutation;
(8) a mutant comprising a combination of at least one engineered disulfide mutation and a least one glycine replacement mutation;
(8) a mutant comprising a combination of at least one engineered disulfide mutation, at least one cavity filling mutation and at least one proline substitution mutation;
(10) a mutant comprising a combination of at least one engineered disulfide mutation, at least one cavity filling mutation, and a least one glycine replacement mutation;
(11) a mutant comprising a combination of at least one engineered disulfide mutation, at least one proline substitution mutation and a least one glycine replacement mutation;
(12) a mutant comprising a combination of at least one engineered disulfide mutation, at least one cavity filling mutation, at least one proline substitution mutation and a least one glycine replacement mutation
(13) a mutant comprising a combination of a cleavage site mutation and at least one engineered disulfide mutation;
(14) a mutant comprising a combination of a cleavage site mutation and at least one cavity filling mutation;
(15) a mutant comprising a combination of a cleavage site mutation and at least one proline substitution mutation;
(16) a mutant comprising a combination of a cleavage site mutation and at least one glycine replacement mutation;
(17) a mutant comprising a combination of a cleavage site mutation and at least one engineered disulfide mutation and at least one cavity filling mutation;
(18) a mutant comprising a combination of a cleavage site mutation and at least one engineered disulfide mutation and at least one proline substitution mutation;
(19) a mutant comprising a combination of a cleavage site mutation and at least one engineered disulfide mutation and a least one glycine replacement mutation;
(20) a mutant comprising a combination of a cleavage site mutation and at least one engineered disulfide mutation, at least one cavity filling mutation and at least one proline substitution mutation;
(21) a mutant comprising a combination of a cleavage site mutation and at least one engineered disulfide mutation, at least one cavity filling mutation, and a least one glycine replacement mutation;
(22) a mutant comprising a combination of a cleavage site mutation and at least one engineered disulfide mutation, at least one proline substitution mutation and a least one glycine replacement mutation;
(23) a mutant comprising a combination of a cleavage site mutation and at least one engineered disulfide mutation, at least one cavity filling mutation, at least one proline substitution mutation and a least one glycine replacement mutation;
(24) a mutant comprising a combination of a cleavage site mutation, at least one cavity filling mutation and at least one proline substitution mutation;
(25) a mutant comprising a combination of a cleavage site mutation, at least one cavity filling mutation and a least one glycine replacement mutation;
(26) a mutant comprising a combination of a cleavage site mutation, at least one proline substitution mutation and at least one glycine replacement mutation;
(27) a combination of at least one cavity filling mutation and at least one proline substitution mutation;
(28) a combination of at least one cavity filling mutation and a least one glycine replacement mutation
(29) a combination of at least one proline substitution mutation and a least one glycine replacement mutation:
(30) a combination of at least one cavity filling mutation, at least one proline substitution mutation and a least one glycine replacement mutation.

In some specific embodiments, the present disclosure provides a nucleic acid molecule which encodes a PIV1 F mutant comprising the mutations selected from the group consisting of:
(1) Q92C-G134C;
(2) A466L;
(3) A466V;
(4) S473V;
(5) S473L;
(7) A466L and S473A;
(8) A466L and S473L;
(10) G134A;
(11) A128P;
(12) F113G, F114S, Q92C-G134C, A466L, S473L and A480L;
(13) Q92C-G134C, A466L, S473L and A480L;
(14) Q92C-G134C, A466L and S473L;
(15) F113G, F114S, Q92C-G134C, A466V, S473V and A480V;
(16) Q92C-G134C, A466V, S473V and A480V;
(17) Q92C-G134C, A466V and S473V;
(18) F113G, F114S, A466L, S473L, A480L and G134A;
(19) A466L, S473L, A480L and G134A;
(20) A466L, S473L and G134A;
(21) F113G, F114S, A466L, S473L, A480L, Q92A and G134A;
(22) F113G, F114S, A466L, S473L and G134A;
(23) A466L, S473L, A480L, Q92A, G134A;
(24) A466L, S473L, Q92A, G134A;
(25) F113G, F114S, Q92L, G134A;
(26) A466L, S473L, A480L, Q92L and G134A;
(27) A466L, S473L, Q92L and G134A;
(28) F113G, F114S, A466L, S473L, A480L, Q92A and G134L;
(29) A466L, S473L, A480L, Q92A and G134L;
(30) F113G, F114S, Q92C-G134C, A4661, S473I and A480L;
(31) F113G, F114S, Q92C-G134C, A466 and, S473I; and,
(32) A4661, S473I, A480L, Q92L and G134A.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length PIV1 F protein mutant disclosed herein comprising the mutations selected from the group consisting of
(1) Q92C-G134C;
(2) A466L;
(3) A466V;
(4) S473V;
(5) S473L;
(7) A466L and S473A;
(8) A466L and S473L;
(10) G134A;
(11) A128P;
(12) F113G, F114S, Q92C-G134C, A466L, S473L and A480L;
(13) Q92C-G134C, A466L, S473L and A480L;
(14) Q92C-G134C, A466L and S473L;
(15) F113G, F114S, Q92C-G134C, A466V, S473V and A480V;
(16) Q92C-G134C, A466V, S473V and A480V;
(17) Q92C-G134C, A466V and S473V;
(18) F113G, F114S, A466L, S473L, A480L and G134A;
(19) A466L, S473L, A480L and G134A;
(20) A466L, S473L and G134A;
(21) F113G, F114S, A466L, S473L, A480L, Q92A and G134A;
(22) F113G, F114S, A466L, S473L and G134A;
(23) A466L, S473L, A480L, Q92A, G134A;
(24) A466L, S473L, Q92A, G134A;
(25) F113G, F114S, Q92L, G134A;
(26) A466L, S473L, A480L, Q92L and G134A;
(27) A466L, S473L, Q92L and G134A;
(28) F113G, F114S, A466L, S473L, A480L, Q92A and G134L;
(29) A466L, S473L, A480L, Q92A and G134L;
(30) F113G, F114S, Q92C-G134C, A4661, S473I and A480L;
(31) F113G, F114S, Q92C-G134C, A4661 and, S473I; and,
(32) A4661, S473I, A480L, Q92L and G134A.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length PIV1 F protein mutant disclosed herein comprising the mutations F113G, F114S, Q92C-G134C, A466L, S473L and A480L.

In some embodiments, the nucleic acid molecule encodes a polypeptide that, when expressed in an appropriate cell, is processed into a disclosed PIV1 HN mutant. In a preferred embodiment, the nucleic acid is an RNA, more preferably an mRNA. In a preferred embodiment, the mRNA encodes a polypeptide that, when expressed in an appropriate cell, is processed into the PIV1 HN protein mutant disclosed herein. In a preferred embodiment, the nucleic acid is an mRNA comprising a chemically modified nucleotide. In a preferred embodiment, the nucleic acid is an mRNA comprising a chemically modified nucleotide, preferably N1-methylpseudouridine. Preferably, all the uridines of the RNA are replaced by N1-methylpseudouridine.

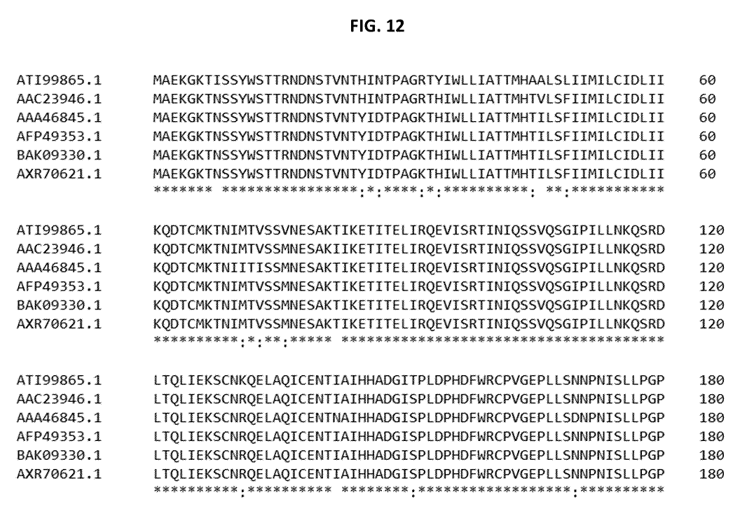

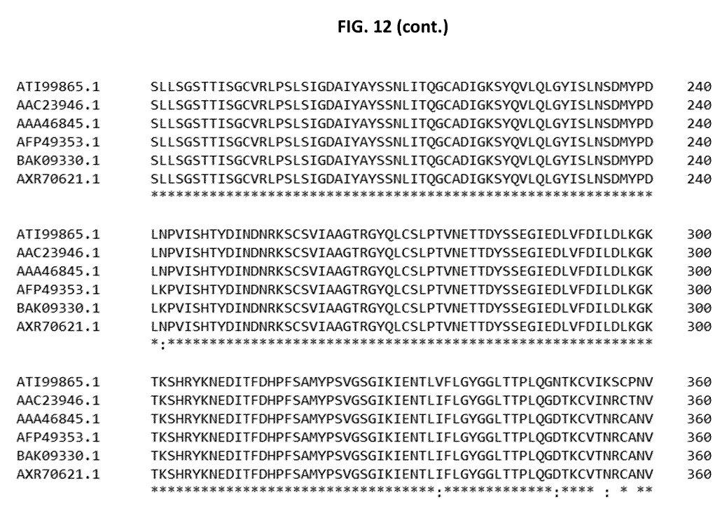

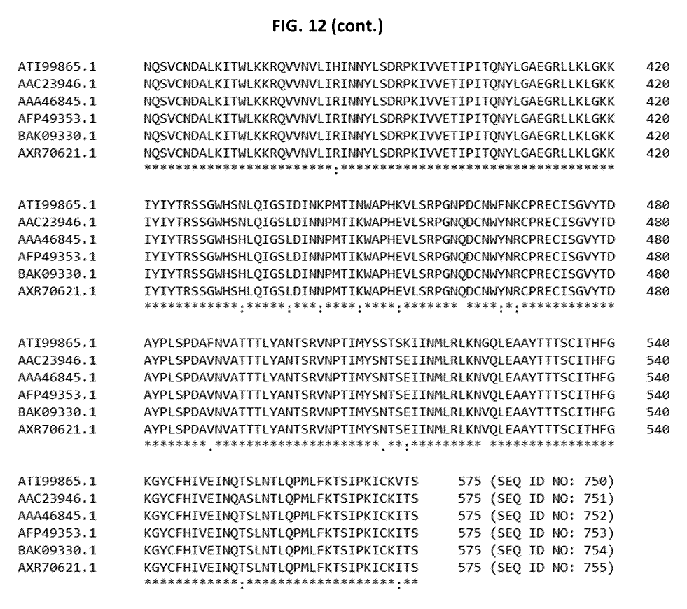

The native PIV1 HN protein is well conserved across strains. For example, any one of the native PIV1 HN strains shown in Table 19 share at least 93% sequence identity with PIV1 HN protein strain MY-U370/12 (Genbank accession number: ATI99865.1) having the sequence set forth in SEQ ID NO: 750. To further illustrate the level of sequence conservation of PIV1 HN proteins, a sequence alignment of the proteins set forth in Table 19 is provided in FIG. 12, which also denotes the positions of the non-consensus amino acids (see also Table 19).

TABLE 19
Sequence similarity across PIV1 HN wildtype protein strains*

| | | Similarity | Dissimilarity/ |
|---|---|---|---|
| | | score | Non-consensus amino |
| Distant isolate/Strain | GenBank ID | (% identity) | acids |
| C39 | AAA46845.1 | 93.91 | N8I Y25H D27N K32R H34Y |
| | | | T45A I46A F49L I71M I73V |
| | | | M76V R131K N142I S151T |
| | | | D170N I335V D349N T354I |
| | | | N355K R356S A358P |
| | | | R385H H433N L439I N443K |
| | | | K448N E453K Q461P |
| | | | Y466F R468K V489F N511S |
| | | | E514K V524G I573V |
| ATCC VR-94 | AFP49353.1 | 94.43 | N8I Y25H D27N K32R H34Y |
| | | | T45A I46A F49L M76V |
| | | | R131K S151T K242N I335V |
| | | | D349N T354I N355K R356S |
| | | | A358P R385H H433N L439I |
| | | | N443K K448N E453K |
| | | | Q461P Y466F R468K V489F |
| | | | N511S E514K V524G I573V |
| C35 | BAK09330.1 | 94.43 | N8I Y25H D27N K32R H34Y |
| | | | T45A I46A F49L M76V |
| | | | R131K S151T K242N I335V |
| | | | D349N T354I N355K R356S |
| | | | A358P R385H H433N L439I |
| | | | N443K K448N E453K |
| | | | Q461P Y466F R468K V489F |
| | | | N511S E514K V524G I573V |
| HPIV1/South Korea/2017 | AXR70621.1 | 94.61 | N8I Y25H D27N K32R H34Y |
| | | | T45A I46A F49L M76V |
| | | | R131K S151T I335V D349N |
| | | | T354I N355K R356S A358P |
| | | | R385H H433N L439I N443K |
| | | | K448N E453K Q461P |
| | | | Y466F R468K V489F N511S |
| | | | E514K V524G I573V |
| HPIV1/Washington/20993/1964 | AAC23946.1 | 95.13 | N8I H34Y T45A V46A F49L |
| | | | M76V I82T R131K S151T |
| | | | I335V D349N N355K R356S |
| | | | T358P R385H L439I N443K |
| | | | K448N E453K Q461P |
| | | | Y466F R468K V489F N511S |
| | | | E514K V524G A553T I573V |

*Sequence similarity as compared to consensus PIV1 HN strain MY-U370/12 (Genbank accession number: ATI99865.1) having the sequence set forth in SEQ ID NO: 750

In one embodiment, the present disclosure provides a nucleic acid molecule which encodes a PIV1 HN protein comprising the mutations selected from the group consisting of:
(1) deletion of amino acids at positions 57-84; and
(2) deletion of amino acids at positions 57-129, wherein the amino acid positions correspond to the positions set forth in wildtype HN polypeptide having SEQ ID NO: 750.

In another embodiment, the present disclosure provides a nucleic acid molecule encoding a PIV1 HN protein, wherein the PIV1 HN protein comprises amino acids having a sequence that is at least 93%, 94%, 95%, 96%, 97%, 98% or 99% identical to the amino acid sequence set forth in SEQ ID NO: 756 or 757.

In another embodiment, the present disclosure provides a PIV1 HN protein mutant comprising the mutations selected from the group consisting of:
(1) deletion of amino acids at positions 57-84; and
(2) deletion of amino acids at positions 57-129,
   wherein the amino acid positions correspond to the positions set forth in wildtype HN polypeptide having SEQ ID NO: 750.

In another embodiment, the present disclosure provides a PIV1 HN protein, wherein the PIV1 HN protein mutant comprising amino acids having a sequence that is at least 93%, 94%, 95%, 96%, 97%, 98% or 99% identical to the amino acid sequence set forth in SEQ ID NO: 756 or 757.

In a preferred embodiment, the present disclosure provides a PIV1 HN protein mutant comprising amino acids having a sequence as set forth in SEQ ID NO: 756 or 757.

In view of the substantial conservation of PIV1 HN protein sequences, a person of ordinary skill in the art can easily compare amino acid positions between different native PIV1 HN protein sequences to identify corresponding PIV1 HN protein amino acid positions between different PIV1 HN strains. Thus, the conservation of native PIV1 HN protein sequences across strains allows use of a reference PIV1 HN sequence for comparison of amino acids at particular positions in the PIV1 HN protein. For the purposes of this disclosure (unless context indicates otherwise), the PIV1 HN protein amino acid positions are given with reference to the sequence of the HN polypeptide set forth in SEQ ID NO: 750 (the amino acid sequence of the full length native polypeptide of the PIV1 HN strain MY-U370/12 (Genbank accession number: ATI99865.1).

However, it should be noted, and one of skill in the art will understand, that different PIV1 HN sequences may have different numbering systems, for example, if there are additional amino acid residues added or removed as compared to SEQ ID NO:750. As such, it is to be understood that when specific amino acid residues are referred to by their number, the description is not limited to only amino acids located at precisely that numbered position when counting from the beginning of a given amino acid sequence, but rather that the equivalent/corresponding amino acid residue in any and all PIV1 HN sequences is intended even if that residue is not at the same precise numbered position, for example if the PIV1 HN sequence is shorter or longer than SEQ ID NO: 750, or has insertions or deletions as compared to SEQ ID NO: 750.

### D. Parainfluenza Virus Type 3 (PIV3)

The present disclosure provides for RNA molecules (e.g., RNA polynucleotides) comprising at least one open reading frame encoding a Parainfluenza Virus Type 3 (PIV3) polypeptide. The present disclosure further provides for an immunogenic composition comprising at least one RNA molecule encoding a PIV3 polypeptide complexed with, encapsulated in, or formulated with one or more lipids, and forming lipid nanoparticles (LNPs). The PIV3 polypeptide encoded by the RNA molecule in the immunogenic composition disclosed herein can be any PIV3 F protein in the prefusion conformation and/or any PIV3 HN protein.

As used herein, a PIV3 F protein or polypeptide or mutant thereof in prefusion conformation may be denoted as “PIV3 preF” or “PIV3 F”. In some embodiments, the PIV3 F protein is a mutant of wild type PIV3 F protein. In some embodiments, the mutants display introduced mutations in the amino acid sequence relative to the amino acid sequence of the corresponding wild-type PIV3 F protein and are immunogenic against the wild-type PIV3 F protein in the prefusion conformation or against a virus comprising the wild-type F protein. The amino acid mutations in the mutants include amino acid substitutions, deletions, or additions relative to a wild-type PIV3 F protein.

In some embodiments, the PIV3 HN protein is a mutant of wild type PIV3 HN protein. In some embodiments, the mutants display introduced mutations in the amino acid sequence relative to the amino acid sequence of the corresponding wild-type PIV3 HN protein and are immunogenic against the wild-type PIV3 HN protein or against a virus comprising the wild-type HN protein. The amino acid mutations in the mutants include amino acid substitutions, deletions, or additions relative to a wild-type PIV3 HN protein.

In some embodiments, the present invention provides nucleic acid molecules that encode a PIV3 F and/or HN protein mutant described herein above. These nucleic acid molecules include DNA, cDNA, and RNA sequences. Nucleic acid molecules that encode only a F2 polypeptide or only a F1 polypeptide of a PIV3 F mutant are also encompassed by the invention. The nucleic acid molecules can be incorporated into a vector, such as an expression vector.

In some embodiments, the nucleic acid molecule encodes a precursor F0 polypeptide that, when expressed in an appropriate cell, is processed into a disclosed PIV3 F mutant. In some embodiments, the nucleic acid molecule encodes a precursor F0 polypeptide that, when expressed in an appropriate cell, is processed into a disclosed PIV3 F mutant, wherein the precursor F0 polypeptide includes, from N- to C-terminus, a signal peptide, a F2 polypeptide, and a F1 polypeptide. In some embodiments, the signal peptide comprises the amino acid sequence set forth as positions 1-18 of any one SEQ ID NOs: 690-694 and 712, wherein the amino acid positions correspond to the amino acid sequence of a reference of SEQ ID NO: 690.

In a preferred embodiment, the nucleic acid is an RNA, more preferably an mRNA. In a preferred embodiment, the mRNA encodes a precursor F0 polypeptide that, when expressed in an appropriate cell, is processed into a full length PIV3 F protein mutant disclosed herein (i.e comprising one or more mutations, a full length F1 polypeptide and a full length F2 polypeptide). A full-length F1 polypeptide of the PIV3 F mutants corresponds to amino acid positions 103-539 of the native PIV3 F0 precursor, and includes (from N- to C-terminus) an extracellular region (residues 103 to 493), a transmembrane domain (residues 494-514), and a cytoplasmic domain (residues 515-539). In a preferred embodiment, the nucleic acid is an mRNA comprising a chemically modified nucleotide. In a preferred embodiment, the nucleic acid is an mRNA comprising a chemically modified nucleotide, preferably 1-methylpseudouridine. Preferably, all the uridines of the RNA are replaced by 1-methylpseudouridine.

In some specific embodiments, the present disclosure provides a nucleic acid molecule which encodes a PIV3 F mutant comprising the mutations selected from the group consisting of:
(1) V175C and A202C;
(2) S160C and V170C;
(3) S164P;
(4) G196A;
(5) G219P;
(6) G230A;
(7) E182L;
(8) S470A;
(9) S477A;
(10) S470A and S477A;
(11) D455S;
(12) A463L;
(13) Q162C, L168C, S470A and S477A;
(14) S160C, V170C, S470A and S477A;
(15) G230A, S470A and S477A;
(16) A463L, S470A and S477A;
(17) E209C and L234C,
(18) A463L and S470L,
(19) S160C, V170C, E209C-L234C, A463L and S470L;
(20) S160C, V170C, E209C, L234C, A463L and 1474F;
(21) S160C, V170C, E209C, L234C, A463L, S470L, F110G, F111S;
(22) S160C, V170C, A463L and S470L;
(23) Q162C, L168C, G230A, A463V and I474Y;
(24) Q162C, L168C, G230A, S470A and S477A;
(25) Q162C, L168C, G230A and A463L;
(26) Q162C, L168C, G230A, A463L, S470A and S477A;
(27) S160C, V170C, G230A, A463V and I474Y;
(28) S160C, V170C, G230A, S470A and S477A;
(29) S160C, V170C, G230A and A463L;
(30) S160C, V170C, G230A, A463L, S470A and S477A; and
(31) S160C, V170C and A463L.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length PIV3 F protein mutant disclosed herein comprising the mutations selected from the group consisting of
(1) V175C and A202C;
(2) S160C and V170C;
(3) S164P;
(4) G196A;
(5) G219P;
(6) G230A;
(7) E182L;
(8) S470A;
(9) S477A;
(10) S470A and S477A;
(11) D455S;
(12) A463L;
(13) Q162C, L168C, S470A and S477A;
(14) S160C, V170C, S470A and S477A;
(15) G230A, S470A and S477A;
(16) A463L, S470A and S477A;
(17) E209C and L234C;
(18) A463L and S470L;
(19) S160C, V170C, E209C, L234C, A463L and S470L;
(20) S160C, V170C, E209C, L234C, A463L and 1474F;
(21) S160C, V170C, E209C, L234C, A463L, S470L, F110G, F111S;
(22) S160C, V170C, A463L and S470L;
(23) Q162C, L168C, G230A, A463V and I474Y;
(24) Q162C, L168C, G230A, S470A and S477A;
(25) Q162C, L168C, G230A and A463L;
(26) Q162C, L168C, G230A, A463L, S470A and S477A;
(27) S160C, V170C, G230A, A463V and I474Y;
(28) S160C, V170C, G230A, S470A and S477A;
(29) S160C, V170C, G230A and A463L;
(30) S160C, V170C, G230A, A463L, S470A and S477A;
(31) S160C, V170C and A463L;
(32) E209C and S233C;
(33) G85C and E209C;
(34) T277V;
(35) A463L and 1474F;
(36) A4631, S4701
(37) S160C, V170C, E209C, S233C, A463L and S470L;
(38) S160C, V170C, E209C, S233C, A463L and 1474F;
(39) S160C, V170C, G85C, E209C, A463L and S470L;
(40) S160C, V170C, G85C, E209C, A463L and 1474F;
(41) S160C, V170C, E209C, L234C, T277V, A463L and S470L;
(42) S160C, V170C, E209C, L234C, T277V, A463L and 1474F;
(43) S160C, V170C, E209C, S233C, T277V, A463L and S470L;
(44) S160C, V170C, E209C, S233C, T277V, A463L and 1474F;
(45) S160C, V170C, G85C, E209C, T277V, A463L and 1474F;
(46) S160C, V170C, E209C, L234C, D455S, A463L and S470L;
(47) S160C, V170C, E209C, S233C, D455S, A463L and S470L;
(48) S160C, V170C, G85C, E209C, D455S, A463L and S470L;
(49) S160C, V170C, E209C, L234C, T277V, D455S, A463L and S470L;
(50) S160C, V170C, E209C, S233C, T277V, D455S, A463L and S470L;
(51) S160C, V170C, G85C, E209C, T277V, D455S, A463L and S470L;
(52) S160C, V170C and S470L;
(53) R106G, T107S, E108A, R109S, S160C, V170C, E209C, L234C, A463L and S470L;
(54) R106G, T107S, E108A, R109S, S160C, V170C, E209C, S233C, A463L and S470L;
(55) R106G, T107S, E108A, R109S, S160C, V170C, G85C, E209C, A463L and S470L;
(56) F110G, F111S, S160C, V170C, E209C, L234C, A463L and S470L;
(57) F110G, F111S, S160C, V170C, E209C, S233C, A463L and S470L;
(58) F110G, F111S, S160C, V170C, A463L and S470L;
(59) F110G, F111S, S160C, V170C and S470L;
(60) S160C, V170C, A463L and S477L;
(61) S160C, V170C, E209C, L234C, A463L and S470L;
(62) S160C, V170C and S470L; and
(63) Q162C, L168C, I213C, G230C, A463V, and I474Y.

In some specific embodiments, the present disclosure provides a nucleic acid molecule, preferably a mRNA, more preferably a mRNA wherein all the uridines are replaced by 1-methylpseudouridine, said nucleic acid encoding a precursor F0 polypeptide that, when expressed in an appropriate cell, is processed into a full length PIV3 F protein mutant disclosed herein comprising the mutations selected from the group consisting of
(1) G230A, S470A and S477A;
(2) S160C, V170C, G230A and A463L;
(3) S160C, V170C, S470A and S477A;
(4) S160C, V170C, G230A, S470A and S477A;
(5) S160C, V170C, G230A, A463L, S470A and S477A;
(6) S160C, V170C, E209C, L234C, A463L and S470L;
(7) S160C, V170C, E209C, L234C, A463L and 1474F;
(8) S160C, V170C, E209C, L234C, A463L, S470L, F110G, F111S; and,
(9) S160C, V170C, A463L and S470L.

In some embodiments, the nucleic acid molecule encodes a polypeptide that, when expressed in an appropriate cell, is processed into a disclosed PIV3 HN mutant. In a preferred embodiment, the nucleic acid is an RNA, more preferably an mRNA. In a preferred embodiment, the mRNA encodes a polypeptide that, when expressed in an appropriate cell, is processed into the PIV3 HN protein mutant disclosed herein. In a preferred embodiment, the nucleic acid is an mRNA comprising a chemically modified nucleotide. In a preferred embodiment, the nucleic acid is an mRNA comprising a chemically modified nucleotide, preferably N1-methylpseudouridine. Preferably, all the uridines of the RNA are replaced by N1-methylpseudouridine.

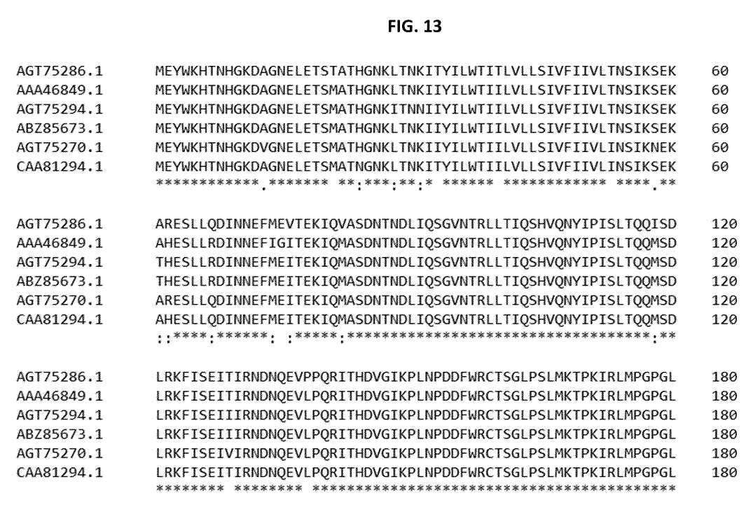

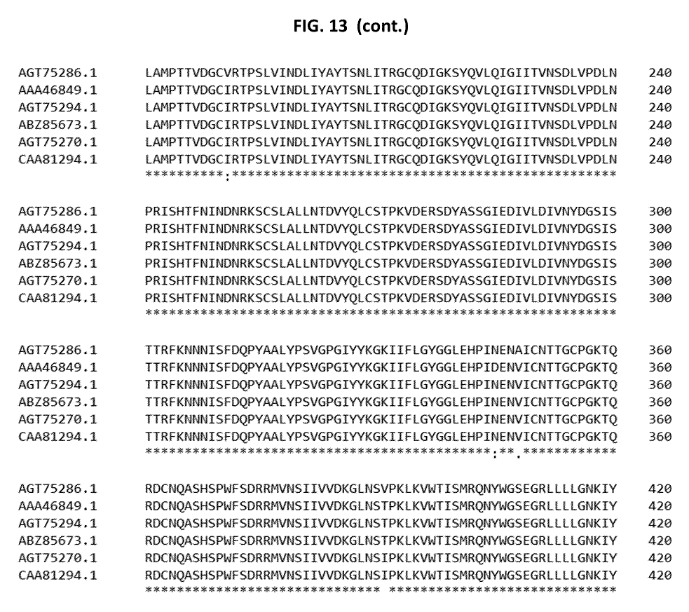

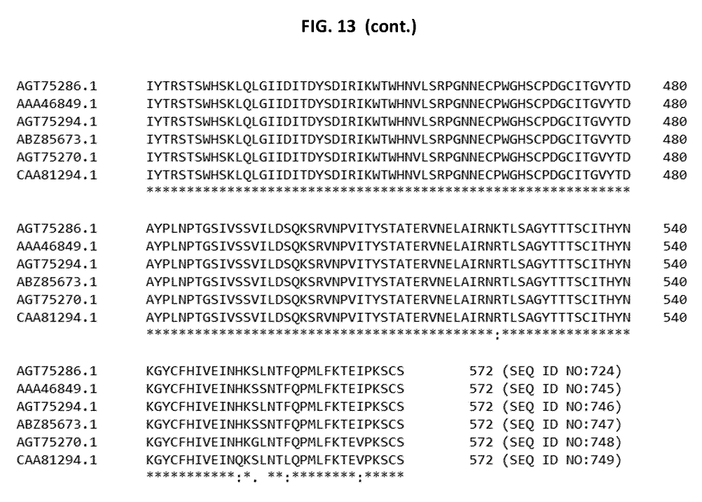

The native PIV3 HN protein is well conserved across strains. For example, any one of the native PIV3 HN strains shown in Table 20 share at least 97% sequence identity with PIV3 HN protein strain HPIV3/MEX/2545/2006 (Genbank accession number: AGT75286.1) having the sequence set forth in SEQ ID NO: 724. To further illustrate the level of sequence conservation of PIV3 HN proteins, a sequence alignment of the proteins set forth in Table 20 is provided in FIG. 13, which also denotes the positions of the non-consensus amino acids (see also Table 20).

TABLE 20
Sequence similarity across PIV3 HN wildtype protein strains*

| | | Similarity | |
|---|---|---|---|
| Distant | | score (% | Dissimilarity/Non-consensus amino |
| isolate/Strain | GenBank ID | identity) | acids |
| HPIV3/AUS/5/2007 | AGT75294.1 | 97.03 | M21T I28L N31K I33T I40T T61A H62R |
| | | | R67Q I76V M82V M118I I129T L138P |
| | | | I191V V348A T391V R524K |
| HPIV3/USA/629- | AGT75270.1 | 97.03 | V13A M21T I33T I40T I53T N58S I76V |
| D02313/2006 | | | M82V M118I V129T L138P I191V V348A |
| | | | I391V R524K G554S V567I |
| HPIV3/TEX/545/80 | AAA46849.1 | 97.03 | M21T I33T I40T H62R R67Q I74M G75E |
| | | | I76V M82V M118I L138P I191V D345N |
| | | | V348A T391V R524K S555L |
| HPIV3/Toronto | CAA81294.1 | 97.2 | M21T N24H I40T I53T H62R I76V M82V |
| | | | M118I L138P I191V V348A I391V R524K |
| | | | Q552H L558F V567I |
| HPIV3/Canada/14702 | ABZ85673.1 | 97.2 | M21T I33T I40T T61A H62R R67Q I76V |
| | | | M82V M118I I129T L138P I191V V348A |
| | | | T391V R524K S555L |

* Sequence similarity as compared to consensus PIV3 HN strain HPIV3/MEX/2545/2006 (Genbank accession number: AGT75286.1) having the sequence set forth in SEQ ID NO: 724

In one embodiment, the present disclosure provides a nucleic acid molecule which encodes a PIV3 HN protein comprising the mutations selected from the group consisting of:
(1) deletion of amino acids at positions 59-88; and
(2) deletion of amino acids at positions 59-130, wherein the amino acid positions correspond to the positions set forth in wildtype HN polypeptide having SEQ ID NO: 724.

In another embodiment, the present disclosure provides a nucleic acid molecule encoding a PIV3 HN protein, wherein the PIV3 HN protein comprises amino acids having a sequence that is at least 97%, 98% or 99% identical to the amino acid sequence set forth in SEQ ID NO: 725 or 726.

In another embodiment, the present disclosure provides a PIV3 HN protein mutant comprising the mutations selected from the group consisting of:
(1) deletion of amino acids at positions 59-88; and
(2) deletion of amino acids at positions 59-130,
   wherein the amino acid positions correspond to the positions set forth in wildtype HN polypeptide having SEQ ID NO: 724.

In another embodiment, the present disclosure provides a PIV3 HN protein, wherein the PIV3 HN protein mutant comprising amino acids having a sequence that is at least 97%, 98% or 99% identical to the amino acid sequence set forth in SEQ ID NO: 725 or 726.

In a preferred embodiment, the present disclosure provides a PIV3 HN protein mutant comprising amino acids having a sequence as set forth in SEQ ID NO: 725 or 726.

In view of the substantial conservation of PIV3 HN protein sequences, a person of ordinary skill in the art can easily compare amino acid positions between different native PIV3 HN protein sequences to identify corresponding PIV3 HN protein amino acid positions between different PIV3 strains. Thus, the conservation of native PIV3 HN protein sequences across strains allows use of a reference PIV3 HN sequence for comparison of amino acids at particular positions in the PIV3 HN protein. For the purposes of this disclosure (unless context indicates otherwise), the PIV3 HN protein amino acid positions are given with reference to the sequence of the HN polypeptide set forth in SEQ ID NO: 724 (the amino acid sequence of the full length native polypeptide of the PIV3 HN strain HPIV3/MEX/2545/2006 (Genbank accession no: AGT75286.1).

However, it should be noted, and one of skill in the art will understand, that different PIV3 HN sequences may have different numbering systems, for example, if there are additional amino acid residues added or removed as compared to SEQ ID NO:724. As such, it is to be understood that when specific amino acid residues are referred to by their number, the description is not limited to only amino acids located at precisely that numbered position when counting from the beginning of a given amino acid sequence, but rather that the equivalent/corresponding amino acid residue in any and all PIV3 HN sequences is intended even if that residue is not at the same precise numbered position, for example if the PIV3 HN sequence is shorter or longer than SEQ ID NO: 724, or has insertions or deletions as compared to SEQ ID NO: 724.

E. Immunogenic Compositions Comprising a Nucleic Acid Encoding a RSV a, RSV B, hMPV A, hMPV B and/or PIV1 and PIV3 F Protein Mutant

There may be situations in which persons are at risk for infection with more than one respiratory viral antigen. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one antigen, a combination vaccine can be administered that includes RNA (e.g., mRNA) encoding at least one antigenic polypeptide protein (or antigenic portion thereof) of a first antigen, e.g. RSV A, RSVB, hMPV A, hMPV B, and/or PIV1 and PIV3 or a fragment thereof, or organism and further includes RNA encoding at least one antigenic polypeptide protein (or antigenic portion thereof) of a second antigen, e.g. RSV A, RSVB, hMPV A, hMPV B and/or PIV1 and PIV3 or a fragment thereof, wherein each RNA (e.g., mRNA) is co-formulated, for example, in a single lipid nanoparticle (LNP) (“pre-mixed”) or can be formulated in separate LNPs for co-administration (“post-mixed”), thereby forming RNA-LNPs.

In one aspect, the invention provides immunogenic compositions that comprise a nucleic acid molecule, preferably modRNA, or vector comprising at least one open reading frame (ORF) encoding a RSV A, RSV B, hMPV A, hMPV B, and/or PIV3 F protein and PIV3 HN protein mutant and/or PIV1 F protein mutant and/or PIV1 HN protein mutant, and a 5′ untranslated region (5′ UTR) set forth in Table 22. In one embodiment, the 5′ UTR comprises a nucleic acid sequence at least 90% identical to a nucleic acid sequence selected from the group consisting of SEQ ID NOs: 336-344, 356-358, 360, 362-367, 370-375, and 378-396.

In one embodiment, the term modRNA, as used in this section, preferably refers to an mRNA encoding a precursor F0 polypeptide that, when expressed in an appropriate cell, is processed into a full length F protein mutant disclosed herein (i.e comprising one or more mutations, a full length polypeptide and a full length F2 polypeptide), preferably wherein all the uridines of the RNA are replaced by a modified base, preferably 1-methylpseudouridine.

In some embodiments, the immunogenic composition comprise one, two, three, four, five or six mutants selected from the group consisting of:
(1) a nucleic acid, preferably a modRNA, encoding a RSV A F protein mutant described in the disclosure;
(2) a nucleic acid, preferably a modRNA, encoding a RSV B F protein mutant described in the disclosure;
(3) a nucleic acid, preferably a modRNA, encoding a hMPV A F protein mutant described in the disclosure;
(4) a nucleic acid, preferably a modRNA encoding a hMPV B F protein mutant described in the disclosure;
(5) a nucleic acid, preferably a modRNA encoding a PIV1 F protein mutant described in the disclosure,
(6) a nucleic acid, preferably a modRNA encoding a PIV1 HN protein mutant described in the disclosure,
(7) a nucleic acid, preferably a modRNA encoding a PIV3 F protein mutant described in the disclosure, and
(8) nucleic acid, preferably a modRNA encoding a PIV3 HN protein mutant described in the disclosure.

In some embodiments, the immunogenic composition comprises one, two, three, four, five or six mutants selected from the group consisting of:
(1) a nucleic acid, preferably a modRNA, encoding a RSV A F protein mutant described in the disclosure;
(2) a nucleic acid, preferably a modRNA, encoding a RSV B F protein mutant described in the disclosure;
(3) a nucleic acid, preferably a modRNA, encoding a hMPV A F protein mutant described in the disclosure;
(4) a nucleic acid, preferably a modRNA encoding a hMPV B F protein mutant described in the disclosure;
(5) a nucleic acid, preferably a modRNA encoding a PIV3 F protein mutant described in the disclosure, and
(6) a nucleic acid, preferably a modRNA encoding a PIV3 HN protein mutant described in the disclosure.

In some embodiments, the immunogenic composition comprises one, two, three, or four mutants selected from the group consisting of:
(1) a nucleic acid, preferably a modRNA, encoding a RSV A F protein mutant described in the disclosure;
(2) a nucleic acid, preferably a modRNA, encoding a RSV B F protein mutant described in the disclosure;
(3) a nucleic acid, preferably a modRNA, encoding a hMPV A F protein mutant described in the disclosure; and
(4) a nucleic acid, preferably a modRNA encoding a hMPV B F protein mutant described in the disclosure.

In some embodiments, the immunogenic composition comprises a nucleic acid, preferably a modRNA, encoding a hMPV A F protein mutant described in the disclosure.

In some embodiments, the immunogenic composition comprises a nucleic acid, preferably a modRNA, encoding a hMPV A F protein mutant described in the disclosure and a hMPV B antigen. In some embodiments, the immunogenic composition comprises a nucleic acid, preferably a modRNA, encoding a hMPV A F protein mutant described in the disclosure and a nucleic acid, preferably a modRNA, encoding a hMPV B F protein mutant described in the disclosure.

In some embodiments, the immunogenic composition comprises a nucleic acid, preferably a modRNA, encoding a hMPV A F protein mutant described in the disclosure and a PIV1 antigen. In some embodiments, the immunogenic composition comprises a nucleic acid, preferably a modRNA, encoding a hMPV A F protein mutant described in the disclosure and a nucleic acid, preferably a modRNA, encoding a PIV1 F protein mutant described in the disclosure.

In some embodiments, the immunogenic composition comprises a nucleic acid, preferably a modRNA, encoding a hMPV A F protein mutant described in the disclosure and a PIV3 antigen. In some embodiments, the immunogenic composition comprises a nucleic acid, preferably a modRNA, encoding a hMPV A F protein mutant described in the disclosure and a nucleic acid, preferably a modRNA, encoding a PIV3 F protein mutant described in the disclosure or a nucleic acid, preferably a modRNA, encoding a PIV3 F protein mutant disclosed in PCT Pub No. WO2018081289 or WO22207839.

In some embodiments, the immunogenic composition comprises a nucleic acid, preferably a modRNA, encoding a hMPV A F protein mutant described in the disclosure, a PIV1 antigen and a PIV3 antigen. In some embodiments, the immunogenic composition comprises a nucleic acid, preferably a modRNA, encoding a hMPV A F protein mutant described in the disclosure, a nucleic acid, preferably a modRNA, encoding a PIV1 F protein mutant described in the disclosure and a nucleic acid, preferably a modRNA, encoding a PIV3 F protein mutant described in the disclosure or disclosed in PCT Pub No. WO2018081289 or WO22207839.

In some embodiments, the immunogenic composition comprises a nucleic acid, preferably a modRNA, encoding a hMPV A F protein mutant described in the disclosure, a hMPV B antigen antigen and a PIV3 antigen.

In some embodiments, the immunogenic composition comprises a nucleic acid, preferably a modRNA, encoding a hMPV A F protein mutant described in the disclosure, a nucleic acid, preferably a modRNA, encoding a hMPV B F protein mutant described in the disclosure and a nucleic acid, preferably a modRNA, encoding a PIV3 F protein mutant described in the disclosure or disclosed in PCT Pub No. WO2018081289 or WO22207839.

In some embodiments, the immunogenic composition comprises a nucleic acid, preferably a modRNA, encoding a hMPV A F protein mutant described in the disclosure, a hMPV B antigen antigen and a PIV1 antigen.

In some embodiments, the immunogenic composition comprises a nucleic acid, preferably a modRNA, encoding a hMPV A F protein mutant described in the disclosure, a nucleic acid, preferably a modRNA, encoding a hMPV B F protein mutant described in the disclosure and a nucleic acid, preferably a modRNA, encoding a PIV1 F protein mutant described in the disclosure.

In some embodiments, the immunogenic composition comprises a nucleic acid, preferably a modRNA, encoding a hMPV A F protein mutant described in the disclosure, a hMPV B antigen, a PIV1 antigen and a PIV3 antigen.

In some embodiments, the immunogenic composition comprises a nucleic acid, preferably a modRNA, encoding a hMPV A F protein mutant described in the disclosure, a nucleic acid, preferably a modRNA, encoding a hMPV B F protein mutant described in the disclosure, a nucleic acid, preferably a modRNA, encoding a PIV3 F protein mutant described in the disclosure or disclosed in PCT Pub No. WO2018081289 or WO22207839 and a nucleic acid, preferably a modRNA, encoding a PIV1 F protein mutant described in the disclosure.

In some embodiments, the immunogenic composition comprises a nucleic acid, preferably a modRNA, encoding a hMPV B F protein mutant described in the disclosure.

In some embodiments, the immunogenic composition comprises a nucleic acid, preferably a modRNA, encoding a hMPV B F protein mutant described in the disclosure and a hMPV A antigen.

In some embodiments, the immunogenic composition comprises a nucleic acid, preferably a modRNA, encoding a hMPV B F protein mutant described in the disclosure and a nucleic acid, preferably a modRNA, encoding a hMPV A F protein mutant described in the disclosure.

In some embodiments, the immunogenic composition a nucleic acid, preferably a modRNA, encoding a hMPV B F protein mutant described in the disclosure and a PIV1 antigen.

In some embodiments, the immunogenic composition comprises or a nucleic acid, preferably a modRNA, encoding a hMPV B F protein mutant described in the disclosure and a nucleic acid, preferably a modRNA, encoding a PIV1 F protein mutant described in the disclosure.

In some embodiments, the immunogenic composition comprises a nucleic acid, preferably a modRNA, encoding a hMPV B F protein mutant described in the disclosure and a PIV3 antigen.

In some embodiments, the immunogenic composition comprises a nucleic acid, preferably a modRNA, encoding a hMPV B F protein mutant described in the disclosure and a nucleic acid, preferably a modRNA, encoding a PIV3 F protein mutant described in the disclosure or a nucleic acid, preferably a modRNA, encoding a PIV3 F protein mutant disclosed in PCT Pub No. WO2018081289 or WO22207839.

In some embodiments, the immunogenic composition comprises a nucleic acid, preferably a modRNA, encoding a hMPV B F protein mutant described in the disclosure, a PIV1 antigen and a PIV3 antigen.

In some embodiments, the immunogenic composition comprises a nucleic acid, preferably a modRNA, encoding a hMPV B F protein mutant described in the disclosure, a nucleic acid, preferably a modRNA, encoding a PIV1 F protein mutant described in the disclosure and a nucleic acid, preferably a modRNA, encoding a PIV3 F protein mutant described in the disclosure or disclosed in PCT Pub No. WO2018081289 or WO22207839.

In some embodiments, the immunogenic composition comprises a nucleic acid, preferably a modRNA, encoding a hMPV B F protein mutant described in the disclosure, a hMPV A antigen antigen and a PIV3 antigen.

In some embodiments, the immunogenic composition comprises a nucleic acid, preferably a modRNA, encoding a hMPV B F protein mutant described in the disclosure, a nucleic acid, preferably a modRNA, encoding a hMPV A F protein mutant described in the disclosure and a nucleic acid, preferably a modRNA, encoding a PIV3 F protein mutant described in the disclosure or disclosed in PCT Pub No. WO2018081289 or WO22207839.

In some embodiments, the immunogenic composition comprises a nucleic acid, preferably a modRNA, encoding a hMPV B F protein mutant described in the disclosure, a hMPV A antigen antigen and a PIV1 antigen.

In some embodiments, the immunogenic composition comprises a nucleic acid, preferably a modRNA, encoding a hMPV B F protein mutant described in the disclosure, a nucleic acid, preferably a modRNA, encoding a hMPV A F protein mutant described in the disclosure and a nucleic acid, preferably a modRNA, encoding a PIV1 F protein mutant described in the disclosure. In some embodiments, the immunogenic composition comprises a nucleic acid, preferably a modRNA, encoding a hMPV B F protein mutant described in the disclosure, a hMPV A antigen, a PIV1 antigen and a PIV3 antigen.

In some embodiments, the immunogenic composition comprises a nucleic acid, preferably a modRNA, encoding a hMPV B F protein mutant described in the disclosure, a nucleic acid, preferably a modRNA, encoding a hMPV A F protein mutant described in the disclosure, a nucleic acid, preferably a modRNA, encoding a PIV3 F protein mutant described in the disclosure or disclosed in PCT Pub No. WO2018081289 or WO22207839 and a nucleic acid, preferably a modRNA, encoding a PIV1 F protein mutant described in the disclosure.

In some embodiments, the immunogenic composition comprises a nucleic acid, preferably a modRNA, encoding a PIV1 F protein mutant described in the disclosure.

In some embodiments, the immunogenic composition comprises a nucleic acid, preferably a modRNA, encoding a PIV1 F protein mutant described in the disclosure and a hMPV A antigen. In some embodiments, the hMPV A antigen is selected from mutants of a wild-type hMPV A F protein and a nucleic acid encoding a mutant of a wild-type hMPV A F protein disclosed in any of PCT Pub No. WO16103238, WO20234300, WO21222639, WO22076669, WO22214678, WO23102373, WO23110618, WO23217988 and WO23102388. In some embodiments, the hMPV A antigen is a nucleic acid encoding a mutant of a wild-type hMPV A F protein comprising the mutations of mutant 115-BV as disclosed in Battles et al, Nature Communication 8:1528 (2017).

In some embodiments, the immunogenic composition comprises a nucleic acid, preferably a modRNA, encoding a PIV1 F protein mutant described in the disclosure and a nucleic acid, preferably a modRNA, encoding a hMPV A protein mutant described in the disclosure.

In some embodiments, the immunogenic composition comprises a nucleic acid, preferably a modRNA, encoding a PIV1 F protein mutant described in the disclosure and a hMPV B antigen. In some embodiments, the hMPV B antigen is selected from mutants of a wild-type hMPV B F protein and a nucleic acid encoding a mutant of a wild-type hMPV B F protein disclosed in any of PCT Pub No. WO16103238, WO20234300, WO21222639, WO22076669, WO22214678, WO23102373, WO23110618, WO23217988 and WO23102388. In some embodiments, the hMPV B antigen is a nucleic acid encoding a mutant of a wild-type hMPV B F protein comprising the mutations of mutant 115-BV as disclosed in Battles et al, Nature Communication 8:1528 (2017).

In some embodiments, the immunogenic composition comprises a nucleic acid, preferably a modRNA, encoding a PIV1 F protein mutant described in the disclosure and a nucleic acid, preferably a modRNA, encoding a hMPV B F protein mutant described in the disclosure. In some embodiments, the immunogenic composition comprises a nucleic acid, preferably a modRNA, encoding a PIV1 F protein mutant described in the disclosure and a PIV3 antigen. In some embodiments, the immunogenic composition comprises a nucleic acid, preferably a modRNA, encoding a PIV1 F protein mutant described in the disclosure and a nucleic acid, preferably a modRNA, encoding a PIV3 F protein mutant described in the disclosure or a nucleic acid, preferably a modRNA, encoding a PIV3 F protein mutant disclosed in PCT Pub No. WO2018081289 or WO22207839.

In some embodiments, the immunogenic composition comprises a nucleic acid, preferably a modRNA, encoding a PIV1 F protein mutant described in the disclosure, a hMPV B antigen and a PIV3 antigen. In some embodiments, the hMPV B antigen is selected from nucleic acids encoding a mutant of a wild-type hMPV B F protein disclosed in any of PCT Pub No. WO16103238, WO20234300, WO21222639, WO22076669, WO22214678, WO23102373, WO23110618, WO23217988 and WO23102388. In some embodiments, the hMPV B antigen is a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a nucleic acid, preferably a modRNA, encoding a PIV1 F protein mutant described in the disclosure, a nucleic acid, preferably a modRNA, encoding a hMPV B F protein mutant described in the disclosure and a nucleic acid, preferably a modRNA, encoding a PIV3 F protein mutant described in the disclosure or disclosed in PCT Pub No. WO2018081289 or WO22207839.

In some embodiments, the immunogenic composition comprises a nucleic acid, preferably a modRNA, encoding a PIV1 F protein mutant described in the disclosure, a hMPV A antigen and a PIV3 antigen. In some embodiments, the hMPV A antigen is selected from nucleic acids encoding a mutant of a wild-type hMPV A F protein disclosed in any of PCT Pub No. WO16103238, WO20234300, WO21222639, WO22076669, WO22214678, WO23102373, WO23110618, WO23217988 and WO23102388. In some embodiments, the hMPV A antigen is a nucleic acid encoding a mutant of a wild-type hMPV A F protein comprising the mutations of mutant 115-BV as disclosed in Battles et al, Nature communication 8:1528 (2017).

In some embodiments, the immunogenic composition comprises a nucleic acid, preferably a modRNA, encoding a PIV1 F protein mutant described in the disclosure, a nucleic acid, preferably a modRNA, encoding a hMPV A F protein mutant described in the disclosure and a nucleic acid, preferably a modRNA, encoding a PIV3 F protein mutant described in the disclosure or disclosed in PCT Pub No. WO2018081289 or WO22207839.

In some embodiments, the immunogenic composition comprises a nucleic acid, preferably a modRNA, encoding a PIV1 F protein mutant described in the disclosure, a hMPV A antigen and a hMPV B antigen. In some embodiments, the hMPV A antigen is selected from a nucleic acids encoding a mutant of a wild-type hMPV A F protein disclosed in any of PCT Pub No. WO16103238, WO20234300, WO21222639, WO22076669, WO22214678, WO23102373, WO23110618, WO23217988 and WO23102388. In some embodiments, the hMPV A antigen is a nucleic acid encoding a mutant of a wild-type hMPV A F protein comprising the mutations of mutant 115-BV as disclosed in Battles et al, Nature communication 8:1528 (2017). In some embodiments, the hMPV B antigen is selected from nucleic acids encoding a mutant of a wild-type hMPV B F protein disclosed in any of PCT Pub No. WO16103238, WO20234300, WO21222639, WO22076669, WO22214678, WO23102373, WO23110618, WO23217988 and WO23102388. In some embodiments, the hMPV B antigen is a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a nucleic acid, preferably a modRNA, encoding a PIV1 F protein mutant described in the disclosure, a nucleic acid, preferably a modRNA, encoding a hMPV A F protein mutant described in the disclosure and a nucleic acid, preferably a modRNA, encoding a hMPV B F protein mutant described in the disclosure.

In some embodiments, the immunogenic composition comprises a nucleic acid, preferably a modRNA, encoding a PIV1 F protein mutant described in the disclosure, a hMPV A antigen, a hMPV B antigen and a PIV3 antigen. In some embodiments, the hMPV A antigen is selected from mutants of nucleic acids encoding a mutant of a wild-type hMPV A F protein disclosed in any of PCT Pub No. WO16103238, WO20234300, WO21222639, WO22076669, WO22214678, WO23102373, WO23110618, WO23217988 and WO23102388. In some embodiments, the hMPV A antigen is a nucleic acid encoding a mutant of a wild-type hMPV A F protein comprising the mutations of mutant 115-BV as disclosed in Battles et al, Nature communication 8:1528 (2017). In some embodiments, the hMPV B antigen is selected from nucleic acids encoding a mutant of a wild-type hMPV B F protein disclosed in any of PCT Pub No. WO16103238, WO20234300, WO21222639, WO22076669, WO22214678, WO23102373, WO23110618, WO23217988 and WO23102388. In some embodiments, the hMPV B antigen is a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a nucleic acid, preferably a modRNA, encoding a PIV1 F protein mutant described in the disclosure, a nucleic acid, preferably a modRNA, encoding a hMPV A F protein mutant described in the disclosure, a nucleic acid, preferably a modRNA, encoding a hMPV B F protein mutant described in the disclosure and a nucleic acid, preferably a modRNA, encoding a PIV3 F protein mutant described in the disclosure or disclosed in PCT Pub No. WO2018081289 or WO22207839.

In some embodiments, the immunogenic composition comprises a nucleic acid, preferably a modRNA, encoding a PIV3 F protein mutant described in the disclosure.

In some embodiments, the immunogenic composition comprises a nucleic acid, preferably a modRNA, encoding a PIV3 F protein mutant described in the disclosure and a hMPV A antigen. In some embodiments, the hMPV A antigen is selected from nucleic acids encoding a mutant of a wild-type hMPV A F protein disclosed in any of PCT Pub No. WO16103238, WO20234300, WO21222639, WO22076669, WO22214678, WO23102373, WO23110618, WO23217988 and WO23102388. In some embodiments, the hMPV A antigen is a nucleic acid encoding a mutant of a wild-type hMPV A F protein comprising the mutations of mutant 115-BV as disclosed in Battles et al, Nature communication 8:1528 (2017).

In some embodiments, the immunogenic composition comprises a nucleic acid, preferably a modRNA, encoding a PIV3 F protein mutant described in the disclosure and a nucleic acid, preferably a modRNA, encoding a hMPV A F protein mutant described in the disclosure.

In some embodiments, the immunogenic composition comprises a nucleic acid, preferably a modRNA, encoding a PIV3 F protein mutant described in the disclosure and a hMPV B antigen. In some embodiments, the hMPV B antigen is selected from nucleic acids encoding a mutant of a wild-type hMPV B F protein disclosed in any of PCT Pub No. WO16103238, WO20234300, WO21222639, WO22076669, WO22214678, WO23102373, WO23110618, WO23217988 and WO23102388. In some embodiments, the hMPV B antigen is a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a nucleic acid, preferably a modRNA, encoding a PIV3 F protein mutant described in the disclosure and a nucleic acid, preferably a modRNA, encoding a hMPV B F protein mutant described in the disclosure.

In some embodiments, the immunogenic composition comprises a nucleic acid, preferably a modRNA, encoding a PIV3 F protein mutant described in the disclosure and a PIV1 antigen.

In some embodiments, the immunogenic composition comprises a nucleic acid, preferably a modRNA, encoding a PIV3 F protein mutant described in the disclosure and a nucleic acid, preferably a modRNA, encoding a PIV3 F protein mutant described in the disclosure.

In some embodiments, the immunogenic composition comprises a nucleic acid, preferably a modRNA, encoding a PIV3 F protein mutant described in the disclosure, a hMPV B antigen and a PIV1 antigen. In some embodiments, the hMPV B antigen is selected from nucleic acids encoding a mutant of a wild-type hMPV B F protein disclosed in any of PCT Pub No. WO16103238, WO20234300, WO21222639, WO22076669, WO22214678, WO23102373, WO23110618, WO23217988 and WO23102388. In some embodiments, the hMPV B antigen is a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a nucleic acid, preferably a modRNA, encoding a PIV3 F protein mutant described in the disclosure, a nucleic acid, preferably a modRNA, encoding a hMPV B F protein mutant described in the disclosure and a nucleic acid, preferably a modRNA, encoding a PIV1 F protein mutant described in the disclosure.

In some embodiments, the immunogenic composition comprises a nucleic acid, preferably a modRNA, encoding a PIV3 F protein mutant described in the disclosure, a hMPV A antigen and a PIV1 antigen. In some embodiments, the hMPV A antigen is selected from nucleic acids encoding a mutant of a wild-type hMPV A F protein disclosed in any of PCT Pub No. WO16103238, WO20234300, WO21222639, WO22076669, WO22214678, WO23102373, WO23110618, WO23217988 and WO23102388. In some embodiments, the hMPV A antigen is a nucleic acid encoding a mutant of a wild-type hMPV A F protein comprising the mutations of mutant 115-BV as disclosed in Battles et al, Nature communication 8:1528 (2017).

In some embodiments, the immunogenic composition comprises a nucleic acid, preferably a modRNA, encoding a PIV3 F protein mutant described in the disclosure, a nucleic acid, preferably a modRNA, encoding a hMPV A F protein mutant described in the disclosure and a nucleic acid, preferably a modRNA, encoding a PIV1 F protein mutant described in the disclosure.

In some embodiments, the immunogenic composition comprises a nucleic acid, preferably a modRNA, encoding a PIV3 F protein mutant described in the disclosure, a hMPV A antigen and a hMPV B antigen. In some embodiments, the hMPV A antigen is selected from nucleic acids encoding a mutant of a wild-type hMPV A F protein disclosed in any of PCT Pub No. WO16103238, WO20234300, WO21222639, WO22076669, WO22214678, WO23102373, WO23110618, WO23217988 and WO23102388. In some embodiments, the hMPV A antigen is a nucleic acid encoding a mutant of a wild-type hMPV A F protein comprising the mutations of mutant 115-BV as disclosed in Battles et al, Nature communication 8:1528 (2017). In some embodiments, the hMPV B antigen is selected from nucleic acids encoding a mutant of a wild-type hMPV B F protein disclosed in any of PCT Pub No. WO16103238, WO20234300, WO21222639, WO22076669, WO22214678, WO23102373, WO23110618, WO23217988 and WO23102388. In some embodiments, the hMPV B antigen is a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a nucleic acid, preferably a modRNA, encoding a PIV3 F protein mutant described in the disclosure, a nucleic acid, preferably a modRNA, encoding a hMPV A F protein mutant described in the disclosure and a nucleic acid, preferably a modRNA, encoding a hMPV B F protein mutant described in the disclosure.

In some embodiments, the immunogenic composition comprises a nucleic acid, preferably a modRNA, encoding a PIV3 F protein mutant described in the disclosure, a hMPV A antigen, a hMPV B antigen and a PIV1 antigen. In some embodiments, the hMPV A antigen is selected from nucleic acids encoding a mutant of a wild-type hMPV A F protein disclosed in any of PCT Pub No. WO16103238, WO20234300, WO21222639, WO22076669, WO22214678, WO23102373, WO23110618, WO23217988 and WO23102388. In some embodiments, the hMPV A antigen is a nucleic acid encoding a mutant of a wild-type hMPV A F protein comprising the mutations of mutant 115-BV as disclosed in Battles et al, Nature communication 8:1528 (2017). In some embodiments, the hMPV B antigen is selected from nucleic acids encoding a mutant of a wild-type hMPV B F protein disclosed in any of WO16103238, WO20234300, WO21222639, WO22076669, WO22214678, WO23102373, WO23110618, WO23217988 and WO23102388. In some embodiments, the hMPV B antigen is a nucleic acid encoding a mutant of a wild-type hMPV B F protein comprising the mutations of mutant 115-BV as disclosed in Battles et al, Nature communication 8:1528 (2017).

In some embodiments, the immunogenic composition comprises a nucleic acid, preferably a modRNA, encoding a PIV3 F protein mutant described in the disclosure, a nucleic acid, preferably a modRNA, encoding a hMPV A F protein mutant described in the disclosure, a nucleic acid, preferably a modRNA, encoding a hMPV B F protein mutant described in the disclosure and a nucleic acid, preferably a modRNA, encoding a PIV1 F protein mutant described in the disclosure.

In some embodiments, the immunogenic composition further comprises a nucleic acid, preferably a modRNA, encoding a PIV3 HN protein mutant described in the disclosure.

In some embodiments, the immunogenic composition further comprises an RSV antigen selected from the group consisting of a nucleic acid, preferably modRNA encoding a mutant of a wild-type RSV F protein of subtype A. Preferably, the mutant is in the form of a trimer. Preferably, the mutant is in the prefusion conformation. Preferably, the mutant is in the prefusion conformation and is in the form of a trimer. Preferably, the RSV antigen is disclosed in one of PCT Pub No. WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220. In some embodiment, the RSV antigen is a nucleic acid, preferably modRNA, encoding a mutant of a wild-type RSV F protein of subtype A comprising the mutations T103C, I148C, S190I, and D486S.

In some embodiments, the composition further comprises an RSV antigen selected from the group consisting of a nucleic acid, preferably modRNA encoding a mutant of a wild-type RSV F protein of subtype B. Preferably, the mutant is in the form of a trimer. Preferably, the mutant is in the prefusion conformation. Preferably, the mutant is in the prefusion conformation and is in the form of a trimer. Preferably, the RSV antigen is disclosed in one of PCT Pub No. WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220. In some embodiment, the RSV antigen a nucleic acid, preferably modRNA, encoding a mutant of a wild-type RSV F protein of subtype B comprising the mutations T103C, I148C, S190I, and D486S.

In some embodiments, the composition further comprises an RSV A antigen selected from the group consisting of a nucleic acid, preferably modRNA encoding a mutant of a wild-type RSV F protein of subtype A and an RSV B antigen selected from the group consisting of a nucleic acid, preferably modRNA encoding a mutant of a wild-type RSV F protein of subtype B. Preferably, the mutants are in the form of a trimer. Preferably, the mutants are in the prefusion conformation. Preferably, the mutants are in the prefusion conformation and is in the form of a trimer. Preferably, the RSV A and B antigens are disclosed in one of PCT Pub No. WO2009/079796, WO2010/149745, WO2011/008974, WO2014/160463, WO2014/174018, WO2014/202570, WO2015/013551, WO2015/177312, WO2017/005848, WO2017/174564, WO2017/005844, WO2017/109629, WO2022/002894 and WO2018/109220. In some embodiment, the RSV A antigen is a nucleic acid, preferably modRNA, encoding a mutant of a wild-type RSV F protein of subtype A comprising the mutations T103C, I148C, S190I, and D486S and the RSV B antigen 15 is a nucleic acid, preferably modRNA, encoding a mutant of a wild-type RSV F protein of subtype B comprising the mutations T103C, I148C, S190I, and D486S.

In some embodiments, the immunogenic composition is capable of eliciting an immune response against the prefusion F protein of hMPV A, hMPV B, PIV1, PIV3, RSV A and/or RSV B an/or the HN protein of PIV3 and/or PIV1 in a subject.

In some embodiments, the immunogenic composition further comprises a pharmaceutically acceptable carrier.

In some embodiments, the immunogenic composition is a vaccine.

In addition to the immunogenic component, the vaccine may further comprise an immunomodulatory agent, such as an adjuvant. Examples of suitable adjuvants include aluminum salts such as aluminum hydroxide and/or aluminum phosphate; oil-emulsion compositions (or oil-in-water compositions), including squalene-water emulsions, such as MF59 (see e.g., WO 90/14837); saponin formulations, such as, for example, QS21 and Immunostimulating Complexes (ISCOMS) (see e.g., U.S. Pat. No. 5,057,540; PCT Pub Nos. WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like. It is also possible to use vector-encoded adjuvant, e.g., by using heterologous nucleic acid that encodes a fusion of the oligomerization domain of C4-binding protein (C4 bp) to the antigen of interest (e.g., Solabomi et al., 2008, Infect Immun 76:3817-23). In certain embodiments the compositions hereof comprise aluminum as an adjuvant, e.g., in the form of aluminum hydroxide, aluminum phosphate, aluminum potassium phosphate, or combinations thereof, in concentrations of 0.05-5 mg, e.g., from 0.075-1.0 mg, of aluminum content per dose.

F. Uses of the RSV A, RSV B, HMPV A, HMPV B and/or PIV1 and/or PIV3 F and/or PIV1 and PIV3 HN Nucleic Acid Molecules and Compositions Thereof

The present disclosure also relates to use of nucleic acids encoding a RSV A, RSV B, hMPV A, hMPV B and/or PIV1 and/or PIV3 F and/or PIV1 HN and/or PIV3 HN protein mutant disclosed herein, or vectors for expressing a RSV A, RSV B, hMPV A, hMPV B and/or PIV1 and PIV3 F and/or PIV1 HN and/or PIV3 HN protein mutant disclosed herein, or compositions comprising a RSV A, RSV B, hMPV A, hMPV B and/or PIV1 and PIV3 F and/or PIV1 HN and/or PIV3 HN nucleic acid disclosed herein.

In several embodiments, the present disclosure provides a method of eliciting an immune response to RSV A, RSV B, hMPV A, hMPV B and/or PIV1 and PIV3 in a subject, comprising administering to the subject an effective amount of a nucleic acid molecule encoding a RSV A, RSV B, hMPV A, hMPV B and/or PIV1 and PIV3 F and/or PIV1 and PIV3 HN protein mutant disclosed herein, or a composition comprising a RSV A, RSV B, hMPV A, hMPV B and/or PIV1 and PIV3 F and/or PIV1 and PIV3 HN nucleic acid molecule disclosed herein.

In some particular embodiments, the present disclosure provides a method of preventing RSV A, RSV B, hMPV A, hMPV B and/or PIV1 and PIV3 infection in a subject, comprising administering to the subject an effective amount of a pharmaceutical composition, such as a vaccine, comprising a nucleic acid encoding a RSV A, RSV B, hMPV A, hMPV B and/or PIV1 and PIV3 F and/or PIV1 and PIV3 HN protein mutant disclosed herein, or a vector expressing a RSV A, RSV B, hMPV A, hMPV B and/or PIV1 and PIV3 F and/or PIV1 and PIV3 HN protein mutant disclosed herein. In some embodiments, the subject is a human. In some particular embodiments, the human is a child, such as an infant. In some other particular embodiments, the human is a woman, particularly a pregnant woman.

In several embodiments, the present disclosure provides a nucleic acid molecule encoding a RSV A, RSV B, hMPV A, hMPV B and/or PIV1 and PIV3 F and/or PIV1 and PIV3 HN protein mutant disclosed herein, or a composition comprising a RSV A, RSV B, hMPV A, hMPV B and/or PIV1 and PIV3 F and/or PIV1 and PIV3 HN nucleic acid molecule disclosed herein for use as a vaccine.

In several embodiments, the present disclosure provides the use of a nucleic acid molecule encoding a RSV A, RSV B, hMPV A, hMPV B and/or PIV1 and PIV3 F and/or PIV1 and PIV3 HN protein mutant disclosed herein, or a composition comprising a hMPV A, hMPV B and/or PIV1 and PIV3 F and/or PIV1 and PIV3 HN protein mutant or nucleic acid molecule disclosed herein for the manufacture of a medicament, preferably a vaccine.

In several embodiments, the present disclosure provides a nucleic acid molecule encoding a RSV A, RSV B, hMPV A, hMPV B and/or PIV1 and PIV3 F and/or PIV1 and PIV3 HN protein mutant disclosed herein, or a composition comprising a nucleic acid molecule disclosed herein for use in a method of eliciting an immune response to RSV A, RSV B, hMPV A, hMPV B and/or PIV1 and PIV3 in a subject, said method comprising administering to the subject an effective amount of said nucleic acid molecule or composition.

In several embodiments, the present disclosure provides a nucleic acid molecule encoding a RSV A, RSV B, hMPV A, hMPV B and/or PIV1 and PIV3 F and/or PIV1 and PIV3 HN protein mutant disclosed herein, or a composition comprising a RSV A, RSV B, hMPV A, hMPV B and/or PIV1 and PIV3 F and/or PIV1 and PIV3 HN nucleic acid molecule disclosed herein for use in preventing RSV A, RSV B, hMPV A, hMPV B and/or PIV1 and PIV3 infection in a subject, said method comprising administering to the subject an effective amount of said protein mutant, nucleic acid molecule or composition.

In some embodiments, the subject is a human. In some particular embodiments, the human is a child, such as an infant. In some other particular embodiments, the human is a woman, particularly a pregnant woman.

The composition may be administered to the subject with or without administration of an adjuvant. The effective amount administered to the subject is an amount that is sufficient to elicit an immune response against an RSV A, RSV B, hMPV A, hMPV B and/or PIV1 and PIV3 antigen, such as RSV A, RSV B, hMPV A, hMPV B and/or PIV1 and PIV3 F protein and/or PIV1 and PIV3 HN, in the subject. Subjects that can be selected for treatment include those that are at risk for developing an RSV A, RSV B, hMPV A, hMPV B and/or PIV1 and PIV3 infection because of exposure or the possibility of exposure to RSV A, RSV B, hMPV A, hMPV B and/or PIV1 and PIV3. Because nearly all humans are infected with RSV A, RSV B, hMPV A, hMPV B and/or PIV1 and PIV3 by the age of 5, the entire birth cohort is included as a relevant population for immunization. This could be done, for example, by beginning an immunization regimen anytime from birth to 6 months of age, from 6 months of age to 5 years of age, in pregnant women (or women of child-bearing age) to protect their infants by passive transfer of antibody, family members of newborn infants or those still in utero, and subjects greater than 50 years of age. Subjects at greatest risk of RSV A, RSV B, hMPV A, hMPV B and/or PIV1 and PIV3 infection with severe symptoms (e.g. requiring hospitalization) include children with prematurity, bronchopulmonary dysplasia, and congenital heart disease.

Administration of the compositions provided by the present disclosure, such as pharmaceutical compositions, can be carried out using standard routes of administration. Non-limiting embodiments include parenteral administration, such as intradermal, intramuscular, subcutaneous, transcutaneous, mucosal, or oral administration.

The total dose of the composition provided to a subject during one administration can be varied as is known to the skilled practitioner.

It is also possible to provide one or more booster administrations of one or more of the vaccine compositions. If a boosting vaccination is performed, typically, such a boosting vaccination will be administered to the same subject at a moment between one week and 10 years, preferably between two weeks and six months, after administering the composition to the subject for the first time (which is in such cases referred to as “priming vaccination”). In alternative boosting regimens, it is also possible to administer different vectors, e.g., one or more adenovirus, or other vectors such as modified vaccinia virus of Ankara (MVA), or DNA, or protein, to the subject after the priming vaccination. It is, for instance, possible to administer to the subject a recombinant viral vector hereof as a prime, and boosting with a composition comprising RSV A, RSV B, hMPV A, HMPV B and/or PIV1 and PIV3 F protein.

In certain embodiments, the administration comprises a priming administration and at least one booster administration. In certain other embodiments, the administration is provided annually. In still other embodiments, the administration is provided annually together with an influenza vaccine.

The vaccines provided by the present disclosure may be used together with one or more other vaccines. For example, in adults they may be used together with an influenza vaccine, Prevnar, tetanus vaccine, diphtheria vaccine, RSV vaccine such as Abryvso™ or Arexvy™, COVID-19 vaccine and pertussis vaccine. For pediatric use, vaccines provided by the present disclosure may be used with any other vaccine indicated for pediatric patients.

### III. RNA Molecule

In some aspects, the RNA molecule described herein is a coding RNA molecule. Coding RNA includes a functional RNA molecule that may be translated into a peptide or polypeptide. In some aspects, the coding RNA molecule includes at least one open reading frame (ORF) coding for at least one peptide or polypeptide. An open reading frame comprises a sequence of codons that is translatable into a peptide or protein. The coding RNA molecule may include one (monocistronic), two (bicistronic) or more (multicistronic) ORFs, which may be a sequence of codons that is translatable into a polypeptide or protein of interest.

The coding RNA molecule may be a messenger RNA (mRNA) molecule, viral RNA molecule, or self-amplifying RNA molecule (saRNA, also referred to as a replicon). In some aspects, the RNA molecule is an mRNA. Preferably, the RNA molecule of the present disclosure is an mRNA. In some aspects, the RNA molecule is modRNA. In some aspects, the RNA molecule is a saRNA. In some aspects, the saRNA molecule may be a coding RNA molecule.

The RNA molecule may encode one polypeptide of interest or more, such as an antigen or more than one antigen, e.g., two, three, four, five, six, seven, eight, nine, ten or more polypeptides. Alternatively, or in addition, one RNA molecule may also encode more than one polypeptide of interest, such as an antigen, e.g., a bicistronic, or tricistronic RNA molecule that encodes different or identical antigens.

The sequence of the RNA molecule may be codon optimized or deoptimized for expression in a desired host, such as a human cell. In some aspects, a gene of interest (e.g., an antigen) described herein is encoded by a coding sequence which is codon-optimized and/or the guanosine/cytidine (G/C) content of which is increased compared to wild type coding sequence. In some aspects, one or more sequence regions of the coding sequence are codon-optimized and/or increased in the G/C content compared to the corresponding sequence regions of the wild type coding sequence. In some aspects, codon-optimization and/or increasing the G/C content does not change the sequence of the encoded amino acid sequence.

The term “codon-optimized” is understood by those in the art to refer to alteration of codons in the coding region of a nucleic acid molecule to reflect the typical codon usage of a host organism without altering the amino acid sequence encoded by the nucleic acid molecule. Within the context of the present disclosure, in some aspects, coding regions are codon-optimized for optimal expression in a subject to be treated using an RNA polynucleotide described herein. Codon-optimization is based on the finding that the translation efficiency is also determined by a different frequency in the occurrence of tRNA molecules in cells. Thus, the sequence of RNA may be modified such that codons for which frequently occurring tRNA molecules are available are inserted in place of “rare codons.”

In some aspects, G/C content of a coding region (e.g., of a gene of interest sequence; open reading frame (ORF)) of an RNA is increased compared to the G/C content of the corresponding coding sequence of a wild type RNA encoding the gene of interest, wherein in some aspects, the amino acid sequence encoded by the RNA is not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability may be determined (so-called alternative codon usage). Depending on the amino acid to be encoded by the RNA, there are various possibilities for modification of the RNA sequence, compared to its wild type sequence. In particular, codons which contain A and/or U nucleosides may be modified by substituting these codons by other codons, which code for the same amino acids but contain no A and/or U or contain a lower content of A and/or U nucleosides. Thus, in some aspects, G/C content of a coding region of an RNA described herein is increased by at least, at most, exactly, or between any two of 10%, 20%, 30%, 40%, 50%, 55%, or even more compared to the G/C content of a coding region of a wild type RNA. In some aspects, the coding region of the RSV RNA described herein comprises a G/C content of at least 50%, at least 55%, at least 60%, at least 65%, at least 70%, at least 75%, or about 80%. In some aspects, the coding region of the RSV RNA described herein comprises a G/C content of about 50% to 75%, about 55% to 70%, about 50% to 60%, about 60% to 70%, about 70% to 80%, about 50% to 55%, about 55% to 60%, about 60% to 65%, about 65% to 70%, about 70% to 75%, or about 75% to 80%. In some aspects, the coding region of the RSV RNA described herein comprises a G/C content of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, or about 75%. In some aspects, the coding region of the RSV RNA described herein comprises a G/C content of about 58%, about 66% or about 62%.

In some aspects, the RNA molecule includes from about 20 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some aspects, the RNA molecule has at least, at most, exactly, or between any two of about 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, 9000, 9200, 9400, 9600, 9800, 10000, 12000, 14000, 16000, 18000, 20000, 22000, 24000, 26000, 28000, 30000, 32000, 34000, 36000, 38000, 40000, 42000, 44000, 46000, 48000, 50000, 52000, 54000, 56000, 58000, 60000, 62000, 64000, 66000, 68000, 70000, 72000, 74000, 76000, 78000, 80000, 82000, 84000, 86000, 88000, 90000, 92000, 94000, 96000, 98000, or 100000 nucleotides.

In some aspects, the RNA molecule includes at least 100 nucleotides. For example, in some aspects, the RNA has a length between 100 and 15,000 nucleotides; between 7,000 and 16,000 nucleotides; between 8,000 and 15,000 nucleotides; between 9,000 and 12,500 nucleotides; between 11,000 and 15,000 nucleotides; between 13,000 and 16,000 nucleotides; between 7,000 and 25,000 nucleotides. In some aspects, the RNA molecule has at least, at most, exactly, or between any two of about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200, 7250, 7300, 7350, 7400, 7450, 7500, 7550, 7600, 7650, 7700, 7750, 7800, 7850, 7900, 7950, 8000, 8050, 8100, 8150, 8200, 8250, 8300, 8350, 8400, 8450, 8500, 8550, 8600, 8650, 8700, 8750, 8800, 8850, 8900, 8950, 9000, 9050, 9100, 9150, 9200, 9250, 9300, 9350, 9400, 9450, 9500, 9550, 9600, 9650, 9700, 9750, 9800, 9850, 9900, 9950, 10000, 10050, 10100, 10150, 10200, 10250, 10300, 10350, 10400, 10450, 10500, 10550, 10600, 10650, 10700, 10750, 10800, 10850, 10900, 10950, 11000, 11050, 11100, 11150, 11200, 11250, 11300, 11350, 11400, 11450, 11500, 11550, 11600, 11650, 11700, 11750, 11800, 11850, 11900, 11950, 12000, 12050, 12100, 12150, 12200, 12250, 12300, 12350, 12400, 12450, 12500, 12550, 12600, 12650, 12700, 12750, 12800, 12850, 12900, 12950, 13000, 13050, 13100, 13150, 13200, 13250, 13300, 13350, 13400, 13450, 13500, 13550, 13600, 13650, 13700, 13750, 13800, 13850, 13900, 13950, 14000, 14050, 14100, 14150, 14200, 14250, 14300, 14350, 14400, 14450, 14500, 14550, 14600, 14650, 14700, 14750, 14800, 14850, 14900, 14950, or 15000 nucleotides.

The RNA molecules of the present disclosure may be prepared by any method know in the art, including chemical synthesis and in vitro methods, such as RNA in vitro transcription. In some of the aspects, the RNA of the present disclosure is prepared using in vitro transcription. In some aspects, the RNA molecule of the present disclosure is purified, e.g., such as by filtration that may occur via, e.g., ultrafiltration, diafiltration, or, e.g., tangential flow ultrafiltration/diafiltration.

In some aspects, the RNA molecule of the present disclosure is lyophilized to be temperature stable.

In some aspects of the present disclosure, an RNA is or comprises messenger RNA (mRNA) that relates to an RNA transcript which encodes a polypeptide. In some aspects, an RNA disclosed herein comprises: a 5′ cap comprising a 5′ cap disclosed herein; a 5′ untranslated region comprising a cap proximal sequence (5′ UTR), a sequence encoding a protein (e.g. polypeptide) (e.g., a RSV prefusion F protein); a 3′ untranslated region (3′ UTR); and/or a polyadenylate (poly-A) sequence.

In some aspects, an RNA disclosed herein comprises the following components in 5′ to 3′ orientation: a 5′ cap comprising a 5′ cap disclosed herein; a 5′ untranslated region comprising a cap proximal sequence (5′ UTR), a sequence encoding a protein (e.g. polypeptide) (e.g., a RSV prefusion F protein); a 3′ untranslated region (3′ UTR); and a poly-A sequence.

In some aspects, an RNA disclosed herein further comprises a signal peptide. Non-limiting examples of signal peptides and amino acid and nucleic acid sequences encoding such peptides can be found in, e.g., WO2017/109629, the disclosure of which is incorporated by reference herein in its entirety.

### A. Modified Nucleobases

In some aspects of the present disclosure, the RNA molecules are not chemically modified and comprise the standard ribonucleotides consisting of adenosine, guanosine, cytosine and uridine. In some aspects, nucleotides and nucleosides of the present disclosure comprise standard nucleoside residues such as those present in transcribed RNA (e.g., A, G, C, and/or U). In some aspects, nucleotides and nucleosides of the present disclosure comprise standard deoxyribonucleosides such as those present in DNA (e.g., dA, dG, dC, and/or dT).

In other aspects of the present disclosure the RNA molecules may comprise modified nucleobases which may be incorporated into modified nucleosides and nucleotides. In some aspects, the RNA molecule may include one or more modified nucleotides. In some aspects, the RNA molecule may include one or more modified nucleotides. Naturally occurring nucleotide modifications are known in the art. In some aspects, the RNA molecule may include a modified nucleotide. Non-limiting examples of modified nucleotides that may be included in the RNA molecule include pseudouridine, N1-methylpseudouridine, 5-methyluridine, 3-methyl-uridine, 5-methoxy-uridine, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), uridine 5-oxyacetic acid, uridine 5-oxyacetic acid methyl ester, 5-carboxymethyl-uridine, 1-carboxymethyl-pseudouridine, 5-carboxy hydroxymethyl-uridine, 5-carboxy 5-hydroxy methyl-uridine methyl ester, methoxycarbonylmethyl-uridine, 5-methoxycarbonylmethyl-2-thio-uridine, 5-aminomethyl-2-thio-uridine, 5-methylaminomethyl-uridine, 1-ethyl-pseudouridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-2-thio-uridine, 1-methyl-4-thio-pseudouridine, 4-thio-1-methyl-pseudouridine, 3-methyl-1-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine, 1-methyl-3-(3-amino-3-carboxypropyl)pseudouridine, 5-(isopentenylaminomethyl)uridine, 5-(isopentenylaminomethyl)-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, any other modified uridine known in the art, or combinations thereof. In some aspects, 1, 2, 3, 4, 5, or more of the foregoing modified nucleotides can be excluded from the RNA molecules disclosed herein.

Modifications that may be present in the RNA molecules further include, but are not limited to, e.g., the following: ms2io6A (2-methylthio-(N6-(cis-hydroxyisopentenyl)adenosine); ms2m6A (2-methylthio-N6-methyladenosine); ms2t6A 2-methylthio-N6-threonylcarbamoyladenosine; g6A (N6-glycinylcarbamoyladenosine); i6A (N6-isopentenyladenosine); m6A (N6-methyladenosine); 16A (N6-threonylcarbamoyladenosine); m′Am (1,2′-O-dimethyladenosine); m1A (1-methyladenosine); 2′-O-methyladenosine; Ar(p) (2′-O-ribosyladenosine (phosphate)); 2-methyl adenosine; 2-methylthio-N6 isopentenyladenosine; ms2hn6A (2-methylthio-N6-hydroxynorvalylcarbamoyladenosine); 2-O-methyladenosine; Am (2-1-O-methyladenosine); 2′-O-ribosyladenosine (phosphate); Isopentenyladenosine; io6A N6-(cis-hydroxyisopentenyl)adenosine; m6Am (N6,2′-O-dimethyladenosine); m62Am (N6,N6,2′-O-trimethyladenosine); m62A (N6,N6-dimethyladenosine); ac6A (N6-acetyladenosine); hn6A (N6-hydroxynorvalylcarbamoyladenosine); m6t6A (N6-methyl-N6-threonylcarbamoyladenosine); m2A (2-methyladenosine); ms2i6A (2-methylthio-N6-isopentenyladenosine); 7-deazaadenosine; N1-methyl-adenosine; N6,N6 (dimethyl) adenine; N6-cis-hydroxy-isopentenyl-adenosine; a-thio-adenosine; 2 (amino) adenine; 2 (aminopropyl) adenine; 2 (methylthio) N6 (isopentenyl) adenine; 2-(alkyl) adenine; 2-(aminoalkyl) adenine; 2-(aminopropyl) adenine; 2-(halo) adenine; 2-(halo) adenine; 2-(propyl) adenine; 2′-amino-2′-deoxy-ATP; 2′-azido-2′-deoxy-ATP; 2′-deoxy-2′-a-aminoadenosine TP; 2′-deoxy-2′-a-azidoadenosine TP; 6 (alkyl) adenine; 6 (methyl) adenine; 6-(alkyl) adenine; 6-(methyl) adenine; 7 (deaza) adenine; 8 (alkenyl) adenine; 8 (alkynyl) adenine; 8 (amino) adenine; 8 (thioalkyl) adenine; 8-(alkenyl) adenine; 8-(alkyl) adenine; 8-(alkynyl) adenine; 8-(amino) adenine; 8-(halo) adenine; 8-(hydroxyl) adenine; 8-(thioalkyl) adenine; 8-(thiol) adenine; 8-azido-adenosine; 8-oxo-adenine; aza adenine; deaza adenine; N6 (methyl) adenine; N6-(isopentyl) adenine; 7-deaza-8-aza-adenosine; 7-methyladenine; 1-deazaadenosine TP; 2′fluoro-N6-Bz-deoxyadenosine TP; 2′-OMe-2-amino-ATP; 2′O-methyl-N6-Bz-deoxyadenosine TP; 2′-a-ethynyladenosine TP; 2-aminoadenine; 2-aminoadenosine TP; 2-amino-ATP; 2′-a-trifluoromethyladenosine TP; 2-azidoadenosine TP; 2′-b-Ethynyladenosine TP; 2-bromoadenosine TP; 2′-b-trifluoromethyladenosine TP; 2-chloroadenosine TP; 2′-deoxy-2′,2′-difluoroadenosine TP; 2′-deoxy-2′-a-mercaptoadenosine TP; 2′-deoxy-2′-a-thiomethoxyadenosine TP; 2′-deoxy-2′-b-aminoadenosine TP; 2′-deoxy-2′-b-azidoadenosine TP; 2′-deoxy-2′-b-bromoadenosine TP; 2′-deoxy-2′-b-chloroadenosine TP; 2′-deoxy-2′-b-fluoroadenosine TP; 2′-deoxy-2′-b-iodoadenosine TP; 2′-deoxy-2′-b-mercaptoadenosine TP; 2′-deoxy-2′-b-thiomethoxyadenosine TP; 2-fluoroadenosine TP; 2-iodoadenosine TP; 2-mercaptoadenosine TP; 2-methoxy-adenine; 2-methylthio-adenine; 2-Trifluoromethyladenosine TP; 3-deaza-3-bromoadenosine TP; 3-deaza-3-chloroadenosine TP; 3-deaza-3-fluoroadenosine TP; 3-deaza-3-iodoadenosine TP; 3-deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-deazaadenosine TP; 2-aminopurine; substituted 7-deazapurine; 7-deaza-7-substituted purine; 7-deaza-8-substituted purine; 7-deaza-2,6-diaminopurine; 7-deaza-8-aza-2,6-diaminopurine; 7-deaza-8-aza-2-aminopurine; 2,4-diaminopurine; 2,6-diaminopurine; 7-deaza-8-aza-adenine; 7-deaza-2-aminopurine; 8-azapurine; s2C (2-thiocytidine); m3C (3-methylcytidine); f5C (5-formylcytidine); hm5C (5-hydroxymethylcytidine); m5C (5-methylcytidine); ac4C (N4-acetylcytidine); Cm (2′-O-methylcytidine); m5Cm (5,2′-O-dimethylcytidine); f5Cm (5-formyl-2′-O-methylcytidine); k2C (Lysidine); m4Cm (N4,2′-O-dimethylcytidine); ac4Cm (N4-acetyl-2′-O-methylcytidine); m4C (N4-methylcytidine); N4,N4-dimethyl-2′-OMe-Cytidine TP; 4-methylcytidine; 5-aza-cytidine; Pseudo-iso-cytidine; pyrrolo-cytidine; a-thio-cytidine; 2-(thio)cytosine; 2′-amino-2′-deoxy-CTP; 2′-azido-2′-deoxy-CTP; 2′-deoxy-2′-a-aminocytidine TP; 2′-deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-chlorocytosine; 5-fluorocytosine; 5-bromocytosine; 5-hydroxycytosine; 5-methylcytosine; 5-(alkyl)cytosine; 5-(alkenyl)cytosine; 5-(alkynyl)cytosine; 5-(halo)cytosine; 5-(propynyl)cytosine; 5-(trifluoromethyl)cytosine; 5-bromocytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′fluor-N4-Bz-cytidine TP; 2′fluoro-N4-Acetyl-cytidine TP; 2′-O-methyl-N4-Acetyl-cytidine TP; 2′O-methyl-N4-Bz-cytidine TP; 2′-a-ethynylcytidine TP; 2′-a-trifluoromethylcytidine TP; 2′-b-Ethynylcytidine TP; 2′-b-Trifluoromethylcytidine TP; 2′-deoxy-2′,2′-difluorocytidine TP; 2′-deoxy-2′-a-mercaptocytidine TP; 2′-deoxy-2′-a-thiomethoxycytidine TP; 2′-deoxy-2′-b-aminocytidine TP; 2′-deoxy-2′-b-azidocytidine TP; 2′-deoxy-2′-b-bromocytidine TP; 2′-deoxy-2′-b-chlorocytidine TP; 2′-deoxy-2′-b-fluorocytidine TP; 2′-deoxy-2′-b-iodocytidine TP; 2′-deoxy-2′-b-mercaptocytidine TP; 2′-deoxy-2′-b-thiomethoxycytidine TP; 2′-O-methyl-5-(1-propynyl)cytidine TP; 3′-ethynylcytidine TP; 4′-azidocytidine TP; 4′-carbocyclic cytidine TP; 4′-ethynyl cytidine TP; 5-(1-propynyl)ara-cytidine TP; 5-(2-chloro-phenyl)-2-thiocytidine TP; 5-(4-Amino-phenyl)-2-thiocytidine TP; 5-Aminoallyl-CTP; 5-cyanocytidine TP; 5-ethynylara-cytidine TP; 5-Ethynylcytidine TP; 5′-Homo-cytidine TP; 5-methoxycytidine TP; 5-Trifluoromethyl-Cytidine TP; N4-Amino-cytidine TP; N4-Benzoyl-cytidine TP; Pseudoisocytidine; mimG (methylguanosine); m7G (7-methylguanosine); m2Gm (N2,2′-O-dimethylguanosine); (N2-methylguanosine); imG (Wyosine); m1Gm (1,2′-O-dimethylguanosine); m1G (1-methylguanosine); 2′-O-methylguanosine; 2′-O-ribosylguanosine (phosphate); Gm (2′-O-methylguanosine); Gr (p) (2′-O-ribosyl guanosine (phosphate)); preQi (7-aminomethyl-7-deazaguanosine); preQo (7-cyano-7-deazaguanosine); G* (Archaeosine); methylwyosine; m2′7G (N2,7-dimethylguanosine); m22Gm (N2,N2,2′-O-trimethylguanosine); m2′2′7G (N2,N2,7-trimethylguanosine); m22G (N2,N2-dimethylguanosine); N2,7,2′-O-trimethylguanosine; 6-thio-guanosine; 7-deaza-guanosine; 8-oxo-guanosine; N1-methyl-guanosine; a-thio-guanosine; 2 (propyl)guanine; 2-(alkyl)guanine; 2′-amino-2′-deoxy-GTP; 2′-azido-2′-deoxy-GTP; 2′-deoxy-2′-a-aminoguanosine TP; 2′-deoxy-2′-a-azidoguanosine TP; N2-dimethylguanine; 6-(methyl)guanine; 6-(alkyl)guanine; 6-(methyl)guanine; 6-methyl-guanosine; 6-thioguanine; 7 (alkyl)guanine; 7-deaza-7-substituted guanine; 7-deaza-7-(C2-c6)alkynylguanine; 7-deaza-8-substituted guanine; 7 (methyl)guanine; 7-(alkyl)guanine; 7-(deaza)guanine; 7-(methyl)guanine; 8-azaguanine; 8-hydroxyguanine; 8-oxoguanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-me-GTP; 2′fluoro-N2-isobutyl-guanosine TP; 2′O-methyl-N2-isobutyl-guanosine TP; 2′-a-ethynylguanosine TP; 2′-a-trifluoromethylguanosine TP; 2′-b-ethynylguanosine TP; 2′-b-trifluoromethylguanosine TP 2′-deoxy-2′,2′-difluoroguanosine TP 2′-deoxy-2′-a-mercaptoguanosine TP; 2′-deoxy-2′-a-thiomethoxyguanosine TP; 2′-deoxy-2′-b-aminoguanosine TP; 2′-deoxy-2′-b-azidoguanosine TP; 2′-deoxy-2′-b-bromoguanosine TP; 2′-deoxy-2′-b-chloroguanosine TP; 2′-deoxy-2′-b-fluoroguanosine TP; 2′-deoxy-2′-b-iodoguanosine TP; 2′-deoxy-2′-b-mercaptoguanosine TP; 2′-deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromoguanosine TP; 9-deazaguanosine TP; N2-isobutyl-guanosine TP; miI (1-methylinosine); I (Inosine); m′Im (1,2′-O-dimethylinosine); 2′-O-methylinosine; 7-methylinosine; Tm (2′-O-methylinosine); oQ (Epoxyqueuosine); galQ (galactosyl-queuosine); manQ (mannosylqueuosine); Q (Queuosine); allyamino-thymidine; aza thymidine; deaza thymidine; deoxy-thymidine; Um (2′-O-methyluridine); s2U (2-thiouridine); m3U (3-methyluridine); cm5U (5-carboxymethyluridine); ho5U (5-hydroxyuridine); m5U (5-methyluridine); tm5s2U (5-taurinomethyl-2-thiouridine); 5-taurinomethyluridine; D (dihydrouridine); pseudouridine; acp3U (3-(3-amino-3-carboxypropyl)uridine); 1-methyl-3-(3-amino-5-carboxypropyl)pseudouridine; 1-methylpseudouridine; 1-ethyl-pseudouridine; 2′-O-methyluridine; 2′-O-methylpseudouridine; 2′-O-methyluridine; s2Um (2-thio-2′-O-methyluridine); 3-(3-amino-3-carboxypropyl)uridine; m3Um (3,2′-O-dimethyluridine); 3-methyl-pseudo-Uridine TP; s4U (4-thiouridine); chm5U (5-(carboxyhydroxymethyl)uridine); mchm5U (5-(carboxyhydroxymethyl)uridine methyl ester); m5Um (5,2′-O-dimethyluridine); 5,6-dihydro-uridine; nm5s2U (5-aminomethyl-2-thiouridine); ncm5Um (5-carbamoylmethyl-2′-O-methyluridine); ncm5U (5-carbamoylmethyluridine); 5-carboxyhydroxymethyluridine; 5-carboxyhydroxymethyluridine methyl ester; cnmm5Um (5-carboxymethylaminomethyl-2′-O-methyluridine); cmnm5s2U (5-carboxymethylaminomethyl-2-thiouridine); 5-carboxymethylaminomethyluridine; cmnm5U (5-carboxymethylaminomethyluridine); 5-Carbamoylmethyluridine TP; mcm5Um (5-methoxycarbonylmethyl-2′-O-methyluridine); mcm5s2U (5-methoxycarbonylmethyl-2-thiouridine); mcm5U (5-methoxycarbonylmethyluridine); mo5U (5-methoxyuridine); m5s2U (5-methyl-2-thiouridine); mnm5se2U (5-methylaminomethyl-2-selenouridine); mnm5s2U (5-methylaminomethyl-2-thiouridine); mnm5U (5-methylaminomethyluridine); m5D (5-methyldihydrouridine); 5-Oxyacetic acid-Uridine TP; 5-Oxyacetic acid-methyl ester-Uridine TP; dihydrouracil; pseudouracil; N1-methyl-pseudo-uracil; N1-ethyl-pseudo-uracil; cmo5U (uridine 5-oxyacetic acid); mcmo5U (uridine 5-oxyacetic acid methyl ester); 3-(3-Amino-3-carboxypropyl)-Uridine TP; 5-(iso-pentenylaminomethyl)-2-thiouridine TP; 5-(iso-pentenylaminomethyl)-2′-O-methyluridine TP; 5-(iso-pentenylaminomethyl)uridine TP; 5-propynyl uracil; a-thio-uridine; 1 (aminoalkylamino-carbonylethylenyl)-2 (thio)-pseudouracil; 1 (aminoalkylamino-carbonylethylenyl)-2,4-(dithio)pseudouracil; 1 (aminoalkylamino-carbonylethylenyl)-4 (thio)pseudouracil; 1 (aminoalkylamino-carbonylethylenyl)-pseudouracil; 1 (aminocarbonylethylenyl)-2 (thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2 (thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-methyl-3-(3-amino-3-carboxypropyl)pseudouridine TP; 1-methyl-3-(3-amino-3-carboxypropyl)pseudo-UTP; 1-methyl-pseudo-UTP; 1-ethyl-pseudo-UTP; 2 (thio)pseudouracil; 2′ deoxy uridine; 2′ fluorouridine; 2-(thio)uracil; 2,4-(dithio)pseudouracil; 2′methyl, 2′amino, 2′azido, 2′fluoro-guanosine; 2′-amino-2′-deoxy-UTP; 2′-azido-2′-deoxy-UTP; 2′-azido-deoxyuridine TP; 2′-O-methylpseudouridine; 2′ deoxyuridine; 2′ fluorouridine; 2′-deoxy-2′-a-aminouridine TP; 2′-deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5-aminouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkenyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2 (thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4 (dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; 5-methyluracil; 5-(hydroxymethyl)uracil; 5-chlorouracil; 5-fluorouracil; 5-bromouracil; N3 (methyl)uracil; pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (+) 1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-trifluoroethyl)-pseudo-UTP; 1-(2,2,3,3,3-pentafluoropropyl)pseudouridine TP; 1-(2,2-diethoxyethyl)pseudouridine TP; 1-(2,4,6-trimethylbenzyl)pseudouridine TP; 1-(2,4,6-Trimethyl-benzyl)pseudo-UTP; 1-(2,4,6-trimethyl-phenyl)pseudo-UTP; 1-(2-amino-2-carboxyethyl)pseudo-UTP; 1-(2-amino-ethyl)pseudo-UTP; 1-(2-Hydroxyethyl)pseudouridine TP; 1-(2-methoxyethyl)pseudouridine TP; 1-(3,4-Bis-trifluoromethoxybenzyl)pseudouridine TP; 1-(3,4-dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 11 (4-Amino-phenyl)pseudo-UTP; 1-(4-azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-iodobenzyl)pseudouridine TP; 1-(4-methanesulfonylbenzyl)pseudouridine TP 1-(4-methoxybenzyl)pseudouridine TP; 1-(4-methoxy-benzyl)pseudo-UTP; 1-(4-methoxy-phenyl)pseudo-UTP; 1-(4-methylbenzyl)pseudouridine TP; 1-(4-methyl-benzyl)pseudo-UTP; 1-(4-nitrobenzyl)pseudouridine TP; 1-(4-Nitro-benzyl)pseudo-UTP; 1 (4-Nitro-phenyl)pseudo-UTP; 1-(4-thiomethoxybenzyl)pseudouridine TP; 1-(4-Trifluoromethoxybenzyl)pseudouridine TP; 1-(4-trifluoromethylbenzyl)pseudouridine TP; 1-(5-Amino-pentyl)pseudo-UTP; 1-(6-Amino-hexyl)pseudo-UTP; 1,6-dimethyl-pseudo-UTP; 1-[3-(2-{2-[2-(2-aminoethoxy)-ethoxy]-ethoxy}-ethoxy)-propionyl]pseudouridine TP; 1-{3-[2-(2-aminoethoxy)-ethoxy]-propionyl}pseudouridine TP; 1-acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-allylpseudouridine TP; 1-Aminomethyl-pseudo-UTP; 1-benzoylpseudouridine TP; 1-benzyloxymethylpseudouridine TP; 1-benzyl-pseudo-UTP; 1-biotinyl-PEG2-pseudouridine TP; 1-biotinylpseudouridine TP; 1-butyl-pseudo-UTP; 1-cyanomethylpseudouridine TP; 1-cyclobutylmethyl-pseudo-UTP; 1-cyclobutyl-pseudo-UTP; 1-cycloheptylmethyl-pseudo-UTP; 1-cycloheptyl-pseudo-UTP; 1-cyclohexylmethyl-pseudo-UTP; 1-cyclohexyl-pseudo-UTP; 1-cyclooctylmethyl-pseudo-UTP; 1-cyclooctyl-pseudo-UTP; 1-cyclopentylmethyl-pseudo-UTP; 1-cyclopentyl-pseudo-UTP; 1-cyclopropylmethyl-pseudo-UTP; 1-cyclopropyl-pseudo-UTP; 1-Ethyl-pseudo-UTP; 1-Hexyl-pseudo-UTP; 1-homoallylpseudouridine TP; 1-hydroxymethylpseudouridine TP; 1-iso-propyl-pseudo-UTP; 1-me-2-thio-pseudo-UTP; 1-me-4-thio-pseudo-UTP; 1-me-alpha-thio-pseudo-UTP; 1-methanesulfonylmethylpseudouridine TP; 1-methoxymethylpseudouridine TP; 1-methyl-6-(2,2,2-Trifluoroethyl)pseudo-UTP; 1-methyl-6-(4-morpholino)-pseudo-UTP; 1-methyl-6-(4-thiomorpholino)-pseudo-UTP; 1-methyl-6-(substituted phenyl)pseudo-UTP; 1-methyl-6-amino-pseudo-UTP; 1-methyl-6-azido-pseudo-UTP; 1-methyl-6-bromo-pseudo-UTP; 1-methyl-6-butyl-pseudo-UTP; 1-methyl-6-chloro-pseudo-UTP; 1-methyl-6-cyano-pseudo-UTP; 1-methyl-6-dimethylamino-pseudo-UTP; 1-methyl-6-ethoxy-pseudo-UTP; 1-methyl-6-ethylcarboxylate-pseudo-UTP; 1-methyl-6-ethyl-pseudo-UTP; 1-methyl-6-fluoro-pseudo-UTP; 1-methyl-6-formyl-pseudo-UTP; 1-methyl-6-hydroxyamino-pseudo-UTP; 1-methyl-6-hydroxy-pseudo-UTP; 1-methyl-6-iodo-pseudo-UTP; 1-methyl-6-iso-propyl-pseudo-UTP; 1-methyl-6-methoxy-pseudo-UTP; 1-methyl-6-methylamino-pseudo-UTP; 1-methyl-6-phenyl-pseudo-UTP; 1-methyl-6-propyl-pseudo-UTP; 1-methyl-6-tert-butyl-pseudo-UTP; 1-methyl-6-trifluoromethoxy-pseudo-UTP; 1-methyl-6-trifluoromethyl-pseudo-UTP; 1-morpholinomethylpseudouridine TP; 1-Pentyl-pseudo-UTP; 1-Phenyl-pseudo-UTP; 1-pivaloylpseudouridine TP; 1-propargylpseudouridine TP; 1-propyl-pseudo-UTP; 1-propynyl-pseudouridine; 1-p-tolyl-pseudo-UTP; 1-tert-butyl-pseudo-UTP; 1-thiomethoxymethylpseudouridine TP; 1-thiomorpholinomethylpseudouridine TP; 1-trifluoroacetylpseudouridine TP; 1-trifluoromethyl-pseudo-UTP; 1-vinylpseudouridine TP; 2,2′-anhydro-uridine TP; 2′-bromo-deoxyuridine TP; 2′-F-5-methyl-2′-deoxy-UTP; 2′-OMe-5-me-UTP; 2′-OMe-pseudo-UTP; 2′-a-ethynyluridine TP; 2′-a-trifluoromethyluridine TP; 2′-b-ethynyluridine TP; 2′-b-trifluoromethyluridine TP; 2′-deoxy-2′,2′-difluorouridine TP; 2′-deoxy-2′-a-mercaptouridine TP; 2′-deoxy-2′-a-thiomethoxyuridine TP; 2′-deoxy-2′-b-aminouridine TP; 2′-deoxy-2′-b-azidouridine TP; 2′-deoxy-2′-b-bromouridine TP; 2′-deoxy-2′-b-chlorouridine TP; 2′-deoxy-2′-b-fluorouridine TP; 2′-deoxy-2′-b-iodouridine TP; 2′-deoxy-2′-b-mercaptouridine TP; 2′-deoxy-2′-b-thiomethoxyuridine TP; 2-methoxy-4-thio-uridine; 2-methoxyuridine; 2′-O-methyl-5-(1-propynyl)uridine TP; 3-Alkyl-pseudo-UTP; 4′-Azidouridine TP; 4′-Carbocyclic uridine TP; 4′-Ethynyluridine TP; 5-(1-propynyl)ara-uridine TP; 5-(2-furanyl)uridine TP; 5-cyanouridine TP; 5-dimethylaminouridine TP; 5′-homo-uridine TP; 5-iodo-2′-fluoro-deoxyuridine TP; 5-phenylethynyluridine TP; 5-trideuteromethyl-6-deuterouridine TP; 5-Trifluoromethyl-Uridine TP; 5-vinylarauridine TP; 6-(2,2,2-Trifluoroethyl)-pseudo-UTP; 6-(4-morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-lodo-pseudo-UTP; 6-iso-Propyl-pseudo-UTP; 6-methoxy-pseudo-UTP; 6-methylamino-pseudo-UTP; 6-methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2 (2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; yW (Wybutosine); OHyW (Hydroxywybutosine); imG2 (isowyosine); 02yW (Peroxywybutosine); OHyW* (undermodified hydroxywybutosine); imG-14 (4-demethylwyosine); 2,6-(diamino) purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino) purine; 2,4,5-(trimethyl)phenyl; 2′methyl, 2′amino, 2′azido, 2′fluoro-cytidine; 2′methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl) isocarbostyrilyl; 3-(methyl) isocarbostyrilyl; 4-(fluoro)-6-(methyl)benzimidazole; 4-(methyl)benzimidazole; 4-(methyl) indolyl; 4,6-(dimethyl) indolyl; 5 nitroindole; 5 substituted pyrimidines; 5-(methyl) isocarbostyrilyl; 5-nitroindole; 6-(aza)pyrimidine; 6-(azo) thymine; 6-(methyl)-7-(aza) indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza) indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl) isocarbostyrilyl; 7-(propynyl) isocarbostyrilyl; propynyl-7-(aza) indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; difluorotolyl; Hypoxanthine; Imidizopyridinyl; Inosinyl; Isocarbostyrilyl; isoguanisine; N2-substituted purines; N6-methyl-2-amino-purine; N6-substituted purines; N-alkylated derivative; napthalenyl; nitrobenzimidazolyl; nitroimidazolyl; nitroindazolyl; nitropyrazolyl; nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; pentacenyl; phenanthracenyl; phenyl; propynyl-7-(aza) indolyl; pyrenyl; pyridopyrimidin-3-yl; pyridopyrimidin-3-yl; 2-oxo-7-amino-pyridopyrimidin-3-yl; pyrrolo-pyrimidin-2-on-3-yl; pyrrolopyrimidinyl; pyrrolopyrizinyl; stilbenzyl; substituted 1,2,4-triazoles; tetracenyl; tubercidine; xanthine; xanthosine-5′-TP; 2-thio-zebularine; 5-aza-2-thio-zebularine; 7-deaza-2-amino-purine; pyridin-4-one ribonucleoside; 2-amino-riboside-TP; formycin A TP; formycin B TP; pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; N6-(19-Amino-pentaoxanonadecyl)adenosine TP; hydrogen (abasic residue); and 2′-O-methyl-U. In some aspects, RNA molecules include a combination of at least two (e.g., 2, 3, 4, or more) of the aforementioned modified nucleobases. In some aspects, 1, 2, 3, 4, 5, or more of the foregoing modifications can be excluded from the RNA molecules disclosed herein.

In some aspects, modified nucleobases in RNA molecules comprise pseudouridine (w), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, N1-methyl-pseudouridine (m1w), 1-ethyl-pseudouridine (e1w), 5-methoxy-uridine (mo5U), 5-methyl-cytidine (m5C), a-thio-guanosine, a-thio-adenosine, 5-cyanouridine, 4′-thio uridine 7-deaza-adenine, 1-methyl-adenosine (m1A), 2-methyl-adenine (m2A), N6-methyl-adenosine (m6A), 2,6-Diaminopurine, inosine (I), 1-methyl-inosine (m1l), wyosine (imG), methylwyosine (mimG), 7-deaza-guanosine, 7-cyano-7-deaza-guanosine (preQO), 7-aminomethyl-7-deaza-guanosine (preQI), 7-methyl-guanosine (m7G), 1-methyl-guanosine (m1G), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropyl)-5,6-dihydrouridine, 3-(3-amino-3-carboxypropyl)pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethyl-2-thiouridine, 5-carboxymethylaminomethyl-2-geranylthiouridine, 5-carboxymethylaminomethyl-2-selenouridine, 5-cyanomethyluridine, 5-hydroxycytidine, 5-methylaminomethyl-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethylcytidine, N6-formyl adenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethyl-2-thiouridine, geranylated 5-carboxymethylaminomethyl-2-thiouridine, Qbase, preQObase, preQlbase, and combinations of two or more thereof. In some aspects, the RNA molecule includes a combination of at least two (e.g., 2, 3, 4, or more) of the aforementioned modified nucleobases, including but not limited to chemical modifications. In some aspects, 1, 2, 3, 4, 5, or more of the foregoing modified nucleobases can be excluded from the RNA molecules disclosed herein.

Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), a-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine. In some aspects, 1, 2, 3, 4, 5, or more of the foregoing modified cytosines can be excluded from the RNA molecules disclosed herein.

In some aspects, a modified nucleobase is a modified uridine. Exemplary nucleobases and nucleosides having a modified uracil include pseudouridine (w), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), 5-cyanouridine, 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnmVU), 5-propynyl-uridine, 1-propynyl-pseudouridine, 5-taurinomethyl-uridine (xm5U), 1-taurinomethyl-pseudouridine, 5-taurinomethyl-2-thio-uridine (xmVu), 1-taurinomethyl-4-thio-pseudouridine, 5-methyl-uridine (m5U, e.g., having the nucleobase deoxythymine), 1-methyl-pseudouridine (m1ψ), 1-ethyl-pseudouridine (e1w), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 w), 5-(isopentenylaminomethyl)uridine (inm5U), 5-(isopentenylaminomethyl)-2-thio-uridine (inm5s2U), a-thio-uridine, 2′-O-methyl-uridine (Um), 5,2′-O-dimethyl-uridine (m5Um), 2′-O-methyl-pseudouridine (Y′m), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl)uridine, and 5-[3-(I-E-propenylamino)]uridine. In some aspects, 1, 2, 3, 4, 5, or more of the foregoing modified uridines can be excluded from the RNA molecules disclosed herein.

In some aspects of the present disclosure, modified nucleotides include any one of N1-methylpseudouridine and/or pseudouridine.

In some aspects, the RNA molecule comprises nucleotides that are N1-methylpseudouridine modified. In some aspects, the RNA molecule comprises nucleotides that are pseudouridine modified.

In some aspects, an RNA comprises a modified nucleoside in place of at least one uridine. In some aspects, an RNA comprises a modified nucleoside in place of each uridine. In some aspects, the RNA molecule comprises a sequence having at least one uridine replaced by N1-methylpseudouridine. In some aspects, the RNA molecule comprises a sequence having all uridines replaced by N1-methylpseudouridine. N1-methylpseudouridine is designated in sequences as “ψ”. The term “uracil,” as used herein, describes one of the nucleobases that may occur in the nucleic acid of RNA. The term “uridine,” as used herein, describes one of the nucleosides that may occur in RNA. “Pseudouridine” is one example of a modified nucleoside that is an isomer of uridine, where the uracil is attached to the pentose ring via a carbon-carbon bond instead of a nitrogen-carbon glycosidic bond.

In some aspects, the RNA molecule comprises a nucleic acid sequence having at least one uridine replaced by N1-methylpseudouridine and/or pseudouridine. In some aspects, the RNA molecule comprises a nucleic acid sequence having at least, at most, exactly, or between (inclusive or exclusive) any two of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of uridines replaced by N1-methylpseudouridine and/or pseudouridine. In some aspects, the RNA molecule comprises a nucleic acid sequence having all uridines replaced by N1-methylpseudouridine and/or pseudouridine.

In some aspects, a modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (16A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyladenosine (g6A), N6-threonylcarbamoyl-adenosine (16A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, a-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar (p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine. In some aspects, 1, 2, 3, 4, 5, or more of the foregoing modified adenines can be excluded from the RNA molecules disclosed herein.

In some aspects, a modified nucleobase is a modified guanine. Exemplary nucleobases and nucleosides having a modified guanine include inosine (I), 1-methyl-inosine (m11), wyosine (imG), methylwyosine (mimG), 4-demethyl-wyosine (imG-14), isowyosine (imG2), wybutosine (yW), peroxywybutosine (02yW), hydroxywybutosine (OhyW), undermodified hydroxywybutosine

(OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQo), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2′7G), N2,N2,7-dimethyl-guanosine (m2′2′7G), 8-oxo-guanosine, 7-methyl-8-oxo-guanosine, 1-methyl-6-thio-guanosine, N2-methyl-6-thio-guanosine, N2,N2-dimethyl-6-thio-guanosine, a-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine, N2,7-dimethyl-2′-O-methyl-guanosine (m2′7Gm), 2′-O-methyl-inosine (Im), 1,2′-O-dimethylinosine (m1Im), 2′-O-ribosylguanosine (phosphate) (Gr (p)), 1-thio-guanosine, 06-methyl-guanosine, 2′-F-ara-guanosine, and 2′-F-guanosine. In some aspects, 1, 2, 3, 4, 5, or more of the foregoing modified guanines can be excluded from the RNA molecules disclosed herein.

In some aspects, RNA molecules are uniformly modified (e.g., fully modified, modified throughout the entire sequence) for a particular modification. In some aspects, the RNA molecules may be partially or fully (e.g., uniformly) modified along the entire length of the molecule. For example, one or more or all or a given type of nucleotide (e.g., purine and/or pyrimidine, or any one or more or all of A, G, U, C) may be uniformly modified in a polynucleotide of the disclosure, or in a given predetermined sequence region thereof. In some aspects, all nucleotides X in a polynucleotide of the present disclosure (or in a given sequence region thereof) are modified nucleotides, wherein X may be any one of nucleotides A, G, U, C, and/or any one of the combinations A+G, A+U, A+C, G+U, G+C, U+C, A+G+U, A+G+C, G+U+C and/or A+G+C. For example, a polynucleotide can be uniformly modified with pseudouridine, meaning that all uridine residues in the RNA sequence are replaced with pseudouridine. Similarly, a polynucleotide can be uniformly modified for any type of nucleoside residue present in the sequence by replacement with a modified residue such as those set forth above. The modified nucleotide can be replaced by a compound having a single unique structure, or can be replaced by a plurality of compounds having different structures (e.g., 2, 3, 4, or more unique structures).

The RNA molecules may contain from or from about 1% to 100% modified nucleotides (either in relation to overall nucleotide content, or in relation to one or more types of nucleotide, e.g., any one or more of A, G, U and/or C) (e.g., at least, at most, exactly, or between (inclusive or exclusive) any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90%) to 100%), and from 95% to 100%). It will be understood that any remaining percentage is accounted for by the presence of unmodified A, G, U, and/or C.

In some aspects, the RNA molecule may include phosphoramidate, phosphorothioate, and/or methylphosphonate linkages.

In some aspects, the RNA molecules may include one or more structural and/or chemical modifications and/or alterations which impart useful properties to the polynucleotide including, in some aspects, reduced degradation in the cell or organism and/or lack of a substantial induction of the innate immune response of a cell into which the RNA molecule is introduced. As used herein, a “structural” feature or modification is one in which two or more linked nucleotides are inserted, deleted, duplicated, inverted and/or randomized in an RNA molecule without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some aspects, a modified RNA molecule, introduced to a cell or organism, exhibits reduced degradation in the cell or organism, respectively, relative to an unmodified nucleic acid comprising standard nucleotides and nucleosides. In some aspects, a modified RNA molecule, introduced into a cell or organism, may exhibit reduced immunogenicity in the cell or organism, respectively (e.g., a reduced innate response) relative to an unmodified nucleic acid comprising standard nucleotides and nucleosides.

In some aspects, the RNA molecule may include one or more modified nucleotides in addition to any 5′ cap structure. In some aspects, the RNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine, which is further described below. In some aspects, the RNA may include a 5′ cap comprising a 7′-methylguanosine, and the first 1, 2, or 3 5′ ribonucleotides may be methylated at the 2′ position of the ribose.

### B. 5′ CAP

In some aspects, the RNA molecule described herein includes a 5′ cap which generally “caps” the 5′ end of the RNA and stabilizes the RNA molecule.

In some aspects, the 5′ cap moiety is a natural 5′ cap. A “natural 5′ cap” is defined as a cap that includes 7-methylguanosine connected to the 5′ end of an mRNA molecule through a 5′ to 5′ triphosphate linkage. In some aspects, a guanosine nucleoside included in a 5′ cap may be modified, for example, by methylation at one or more positions (e.g., at the 7-position) on a base (guanine), and/or by methylation at one or more positions of a ribose. In some aspects, a guanosine nucleoside included in a 5′ cap comprises a 3′O methylation at a ribose (3′OMeG). In some aspects, a guanosine nucleoside included in a 5′ cap comprises methylation at the 7-position of guanine (m7G). In some aspects, a guanosine nucleoside included in a 5′ cap comprises methylation at the 7-position of guanine and a 3′O methylation at a ribose (m7(3′OMeG)). The 5′ cap may be incorporated during RNA synthesis (e.g., co-transcriptional capping) or may be enzymatically engineered after RNA transcription (e.g., post-transcriptional capping). In some aspects, co-transcriptional capping with a cap disclosed herein improves the capping efficiency of an RNA compared to co-transcriptional capping with an appropriate reference comparator. In some aspects, improving capping efficiency may increase a translation efficiency and/or translation rate of an RNA, and/or increase expression of an encoded polypeptide. In some aspects, capping is performed after purification, e.g., tangential flow filtration, of the RNA molecule.

In some aspects, an RNA described herein comprises a 5′ cap or a 5′ cap analog, e.g., a Cap 0, a Cap 1 or a Cap 2. In some aspects, a provided RNA does not have uncapped 5′-triphosphates. In some aspects, the 5′ end of the RNA is capped with a modified ribonucleotide. In some aspects, the 5′ cap moiety is a 5′ cap analog. In some aspects, an RNA may be capped with a 5′ cap analog. Cap structures include, but are not limited to, 7 mG (5′)ppp(5′) N1pN2p (Cap 0), 7 mG (5′)ppp(5′) N1mpNp (Cap 1), and 7 mG (5′)ppp(5′) N1mpN2mp (Cap 2). In some aspects, 1, 2, 3, 4, 5, or more of the foregoing cap structures can be excluded from the RNA molecules disclosed herein.

In some aspects, an RNA described herein comprises a Cap 0. In some aspects, Cap 0 is a N7-methyl guanosine, and a Cap 0 structure comprises a guanosine nucleoside methylated at the 7-position of guanine (m7G). In some aspects, a Cap 0 structure is connected to an RNA via a 5′ to 5′-triphosphate linkage and is also referred to herein as m7G, m7Gppp, and/or m7G(5′)ppp(5′). A 5′ cap may be methylated with the structure ⁷mG(5′)ppp(5′)N₁pN₂p (Cap 0) or a derivative thereof, wherein N is the terminal 5′ nucleotide of the nucleic acid carrying the 5′ cap, typically the 5′-end of an mRNA. An exemplary enzymatic reaction for capping may include use of Vaccinia Virus Capping Enzyme (VCE) that includes mRNA triphosphatase, guanylyltransferase and guanine-7-methytransferase, which catalyzes the construction of N7-monomethylated Cap 0 structures. Cap 0 structures play an important role in maintaining the stability and translational efficacy of the RNA molecule. In the cell, the Cap 0 structure is essential for efficient translation of the mRNA that carries the cap.

In some aspects, an RNA described herein comprises a Cap 1, e.g., as described herein. The 5′ cap of the RNA molecule may be further modified on the 2′O position by a 2′-O-methyltransferase, which results in the generation of a Cap 1 structure (m7Gppp [m2′-O] N), which may further increase translation efficacy. In some aspects, a Cap 1 structure comprises a guanosine nucleoside methylated at the 7-position of guanine (m7G) and a 2′O methylated first nucleotide in an RNA (2′OMeN₁). In some aspects, a Cap 1 structure is connected to an RNA via a 5′- to 5′-triphosphate linkage and is also referred to herein as m7GpppN^{m}, wherein Nm denotes any nucleotide with a 2′O methylation, ⁷mG(5′)ppp(5′)N₁^{m}pNp, m7Gppp(2′OMeN₁), and/or m7G(5′)ppp(5′)(2′OMeN₁). In some aspects, N₁ is chosen from A, C, G, or U. In some aspects, N₁ is A. In some aspects, N₁ is C. In some aspects, N₁ is G. In some aspects, N₁ is U. In some aspects, a m7G(5′)ppp(5′)(2′OMeN₁) Cap 1 structure comprises a second nucleotide, N₂, which is a cap proximal nucleotide at position 2 and is chosen from A, G, C, or U (m7G(5′)ppp(5′)(2′OMeN₁)N₂). In some aspects, N₂ is A. In some aspects, N₂ is C. In some aspects, N₂ is G. In some aspects, N₂ is U.

In some aspects, a Cap 1 structure comprises a guanosine nucleoside methylated at the 7-position of guanine (m7G) and one or more additional modifications, e.g., methylation on a ribose, and a 2′O methylated first nucleotide in an RNA. In some aspects, a Cap 1 structure comprises a guanosine nucleoside methylated at the 7-position of guanine, a 3′O methylation at a ribose (m7(3′OMeG)), and a 2′O methylated first nucleotide in an RNA (2′OMeN₁). In some aspects, a Cap 1 structure is connected to an RNA via a 5′- to 5′-triphosphate linkage and is also referred to herein as m7(3′OMeG)ppp(2′OMeN₁) and/or m7(3′OMeG)(5′)ppp(5′)(2′OMeN₁). In some aspects, N₁ is chosen from A, C, G, or U. In some aspects, N₁ is A. In some aspects, N₁ is C. In some aspects, N₁ is G. In some aspects, N₁ is U. In some aspects, a m7(3′OMeG)(5′)ppp(5′)(2′OMeN₁) Cap 1 structure comprises a second nucleotide, N₂, which is a cap proximal nucleotide at position 2 and is chosen from A, G, C, or U (m7(3′OMeG)(5′)ppp(5′)(2′OmeN₁)N₂). In some aspects, N₂ is A. In some aspects, N₂ is C. In some aspects, N₂ is G. In some aspects, N₂ is U. In some aspects, 1, 2, 3, 4, 5, or more of the foregoing Cap 1 structures can be excluded from the RNA molecules disclosed herein.

In some aspects, a second nucleotide in a Cap 1 structure may comprise one or more modifications, e.g., methylation. In some aspects, an RNA described herein comprises a Cap 2. In some aspects, a Cap 1 structure comprising a second nucleotide comprising a 2′O methylation is a Cap 2 structure.

In some aspects, the RNA molecule may be enzymatically capped at the 5′ end using Vaccinia guanylyltransferase, guanosine triphosphate, and S-adenosyl-L-methionine to yield Cap 0 structure. An inverted 7-methylguanosine cap is added via a 5′ to 5′ triphosphate bridge. Alternatively, use of a 2′O-methyltransferase with Vaccinia guanylyltransferase yields the Cap 1 structure where, in addition to the Cap 0 structure, the 2′OH group is methylated on the penultimate nucleotide. S-adenosyl-L-methionine (SAM) is a cofactor utilized as a methyl transfer reagent. Non-limiting examples of 5′ cap structures are those which, among other things, have enhanced binding of cap-binding polypeptides, increased half-life, reduced susceptibility to 5′ endonucleases and/or reduced 5′ decapping, as compared to synthetic 5′ cap structures known in the art (or to a wild type, natural or physiological 5′ cap structure).

For example, recombinant Vaccinia Virus Capping Enzyme and recombinant 2′ O-methyltransferase enzyme may create a canonical 5′-5′-triphosphate linkage between the 5′-terminal nucleotide of an mRNA and a guanine cap nucleotide wherein the cap guanine includes an N7 methylation and the 5′-terminal nucleotide of the mRNA includes a 2′-O-methyl. Such a structure is termed the Cap 1 structure. This cap results in a higher translational-competency and cellular stability and a reduced activation of cellular pro-inflammatory cytokines, as compared, e.g., to other 5′ cap analog structures known in the art.

A cap species may include one or more modified nucleosides and/or linker moieties. For example, a cap may include a guanine nucleotide and a guanine (G) nucleotide methylated at the 7 position joined by a triphosphate linkage at their 5′ positions, e.g., m7G (5′)ppp(5′) G, commonly written as m7GpppG. A cap species may also be an anti-reverse cap analog. A non-limiting list of possible cap species includes m7GpppG, m7Gpppm7G, m73′dGpppG, m27,03′GpppG, m27,03′GppppG, m7Gpppm7G, m73′dGpppG, m27,03′GpppG, m27,03′GppppG, and m27,02′GppppG. In some aspects, 1, 2, 3, 4, 5, or more of the foregoing cap species can be excluded from the RNA molecules disclosed herein.

In some aspects, the 5′ terminal cap includes a cap analog, for example, a 5′ terminal cap may include a guanine analog. Exemplary guanine analogs include, but are not limited to, inosine, N1-methyl-guanosine, 2′-fluoro-guanosine, 7-deaza-guanosine, 8-oxo-guanosine, 2-aminoguanosine, LNA-guanosine, and 2-azido-guanosine. In some aspects, 1, 2, 3, 4, 5, or more of the foregoing guanine analogs can be excluded from the cap structures disclosed herein.

In some aspects, the capping region may include a single cap or a series of nucleotides forming the cap. In this aspect the capping region may be from 1 to 10, e.g., 2-9, 3-8, 4-7, 1-5, 5-10, or at least 2, or 10 or fewer nucleotides in length. In this aspect, the capping region is at least, at most, exactly, or between (inclusive or exclusive) any two of 1, 2, 3, 4, 5, 6, 7, 8, 9, or 10 nucleotides in length. In some aspects, the cap is absent. In some aspects, the first and second operational regions may range from 3 to 40, e.g., 5-30, 10-20, 15, or at least 4, or 30 or fewer nucleotides in length and may comprise, in addition to a Start and/or Stop codon, one or more signal and/or restriction sequences. In some aspects, the first and second operational regions are at least, at most, exactly, or between (inclusive or exclusive) any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length and may comprise, in addition to a Start and/or Stop codon, one or more signal and/or restriction sequences.

Further examples of 5′ cap structures include, but are not limited to, glyceryl, inverted deoxy abasic residue (moiety), 4′, 5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′-phosphorothioate, phosphorodithioate, and/or bridging or non-bridging methylphosphonate moiety. In some aspects, 1, 2, 3, 4, 5, or more of the foregoing 5′ cap structures can be excluded from the RNA molecules disclosed herein.

In some aspects, the RNA molecule of the present disclosure comprises at least one 5′ cap structure. In some aspects, the RNA molecule of the present disclosure does not comprise a 5′ cap structure.

Numerous synthetic 5′ cap analogs have been developed and are known in the art to enhance mRNA stability and translatability (see, e.g., Grudzien-Nogalska, E., Kowalska, J., Su, W., Kuhn, A. N., Slepenkov, S. V., Darynkiewicz, E., Sahin, U., Jemielity, J., and Rhoads, R. E., Synthetic mRNAs with superior translation and stability properties in Synthetic Messenger RNA and Cell Metabolism Modulation in Methods in Molecular Biology 69 (Rabinovich, P. H. Ed), 2013). In one aspect, the 5′ capping structure comprises a modified 5′ Cap 1 structure (m7G+m3′-5′-ppp-5′-Am). In one aspect, the 5′ capping structure is (3′OMe)-m₂^{7,3′-O}Gppp(m₁^{2′-O})ApG (TriLink BioTechnologies) having the structure set forth below:

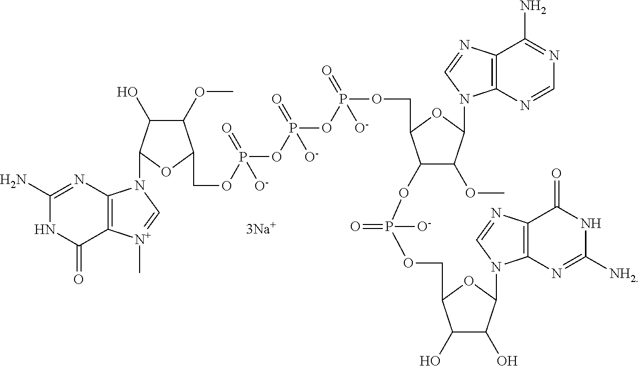 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

This molecule is identical to the natural RNA cap structure in that it starts with a guanosine methylated at N7, and is linked by a 5′ to 5′ triphosphate linkage to the first coded nucleotide of the transcribed RNA (in this case, an adenosine). This guanosine is also methylated at the 3′ hydroxyl of the ribose to mitigate possible reverse incorporation of the cap molecule. The 2′ hydroxyl of the ribose on the adenosine is methylated conferring a Cap 1 structure.

### C. Untranslated Regions (UTRs)

An untranslated region (UTR) is located on either side of a coding sequence on an mRNA molecule and is involved in many regulatory aspects of gene expression. A 5′ UTR (or leader sequence) is present upstream from the initiation codon on the 5′ side of the coding sequence and a 3′ UTR is present immediately following the translation termination codon on the 3′ side of the coding sequence. The 5′ UTR contains regulatory elements which regulate protein expression and the 3′ UTR contains regulatory regions that post-transcriptionally influence gene expression and affect mRNA half-life.

In some aspects, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). Accordingly, the UTR sequence may prolong protein synthesis in a tissue-specific manner.

In some aspects, the regulatory features of a UTR can be incorporated into the RNAs of the present disclosure to, among other things, enhance the stability of the molecule. The specific features can also be incorporated to ensure controlled down-regulation of the transcript in case they are misdirected to undesired organs sites

It should be understood that any UTR from any gene may be incorporated into the regions of the RNAs of the present disclosure. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation and/or location. Hence a 5′ and/or 3′ UTR may be inverted, shortened, lengthened, and/or made with one or more other 5′ UTRs or 3′ UTRs. As used herein, the term “altered” as it relates to a UTR sequence, means that the UTR has been changed in some way in relation to a reference sequence. For example, 5′ UTRs and/or 3′ UTRs may be altered relative to a wild-type or native UTR by the change in orientation and/or location as taught above and/or may be altered by the inclusion of additional nucleotides, deletion of nucleotides, swapping, and/or transposition of nucleotides. Any of these changes produces an “altered” UTR (whether 5′ and/or 3′) including a variant UTR.

In some embodiments, a double, triple or quadruple UTR such as a 5′ and/or 3′ UTR may be used. As used herein, a “double” UTR is one in which two copies of the same UTR are encoded either in series or substantially in series. For example, a double beta-globin 3′ UTR may be used. It is also within the scope of the present disclosure to have patterned UTRs. As used herein “patterned UTRs” are those UTRs which reflect a repeating or alternating pattern, such as AB AB AB or AABBAABBAABB or ABCABCABC or variants thereof repeated once, twice, or more than 3 times. In these patterns, each letter, A, B, or C represent a different UTR at the nucleotide level.

RNAs may encode polypeptides of interest belonging to a family of proteins that are expressed in a particular cell, tissue and/or at some time during development. In some aspects, the UTRs from any of these genes may be swapped for any other UTR of the same or different family of proteins to create a new RNA molecule. As used herein, a “family of proteins” is used in the broadest sense to refer to a group of two or more polypeptides of interest which share at least one function, structure, feature, localization, origin, and/or expression pattern.

In some aspects, the 5′ UTR and the 3′ UTR sequences are computationally derived. In some aspects, the 5′ UTR and the 3′ UTRs are derived from a naturally abundant mRNA in a tissue. The tissue may be, for example, liver, a stem cell and/or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, and/or a natural killer cell), a macrophage, a monocyte, a dendritic cell, a neutrophil, an eosinophil and a reticulocyte. In some aspects, the 5′ UTR and the 3′ UTR are derived from an alphavirus. In some aspects, the 5′ UTR and the 3′ UTR are from a wild type alphavirus.

In some aspects, untranslated regions may also include translation enhancer elements (TEE). As a non-limiting example, the TEE may include those described in U.S. application No. 20090226470, herein incorporated by reference in its entirety, and those known in the art.

Those of ordinary skill in the art will understand that 5′ UTRs that are heterologous and/or synthetic may be used with any desired 3′ UTR sequence, and vice versa. For example, a heterologous 5′ UTR may be used with a synthetic and/or heterologous 3′ UTR.

In some aspects, an RNA molecule disclosed herein comprises a 5′ UTR selected from Table 22. A 5′ UTR, if present, is located at the 5′ end and starts with the transcriptional start site upstream of the start codon of a protein encoding region. A 5′ UTR is downstream of the 5′ cap (if present), e.g. directly adjacent to the 5′ cap. The 5′ UTR may contain various regulatory elements, e.g., 5′ cap structure, stem-loop structure, and an internal ribosome entry site (IRES), which may play a role in the control of translation initiation. The 5′ UTR may harbor signatures like Kozak sequences, which are also involved in the process by which the ribosome initiates translation of many genes. 5′ UTRs may also form secondary structures involved in elongation factor binding.

In some aspects, a 5′ UTR disclosed herein comprises a cap proximal sequence, e.g., as disclosed herein. In some aspects, a cap proximal sequence comprises a sequence adjacent to a 5′ cap. In some aspects, a cap proximal sequence comprises nucleotides in positions +1, +2, +3, +4, and/or +5 of an RNA polynucleotide.

In some aspects, a Cap structure comprises one or more polynucleotides of a cap proximal sequence. In some aspects, a Cap structure comprises an m7 Guanosine cap and nucleotide +1 (N₁) of an RNA polynucleotide. In some aspects, a Cap structure comprises an m7 Guanosine cap and nucleotide +2 (N₂) of an RNA polynucleotide. In some aspects, a Cap structure comprises an m7 Guanosine cap and nucleotides +1 and +2 (N₁ and N₂) of an RNA polynucleotide.

Those skilled in the art, reading the present disclosure, will appreciate that, in some aspects, one or more residues of a cap proximal sequence (e.g., one or more of residues +1, +2, +3, +4, and/or +5) may be included in an RNA by virtue of having been included in a cap entity that (e.g., a Cap 1 structure, etc); alternatively, in some aspects, at least some of the residues in a cap proximal sequence may be enzymatically added (e.g., by a polymerase such as a T7 polymerase). For example, in certain exemplified aspects where a (m₂^{7,3′-O})Gppp(m^{2′-O})ApG cap is utilized, +1 and +2 residues are the (m₂^{7,3′-O}) A and G residues of the cap, and +3, +4, and +5 residues are added by polymerase (e.g., T7 polymerase).

In some aspects, a cap proximal sequence comprises N₁ and/or N₂ of a Cap structure, wherein N₁ and N₂ are any nucleotide, e.g., A, C, G or U. In some aspects, N₁ is A. In some aspects, N₁ is C. In some aspects, N₁ is G. In some aspects, N₁ is U. In some aspects, N₂ is A. In some aspects, N₂ is C. In some aspects, N₂ is G. In some aspects, N₂ is U. In some aspects, a cap proximal sequence comprises N₁ and N₂ of a Cap structure and N₃, N₄ and N₅, wherein N₁ to N₅ correspond to positions +1, +2, +3, +4, and/or +5 of an RNA polynucleotide. In some aspects, N₁, N₂, N₃, N₄, or N₅ are any nucleotide, e.g., A, C, G or U. In some aspects, N₁N₂ comprises any one of the following: AA, AC, AG, AU, CA, CC, CG, CU, GA, GC, GG, GU, UA, UC, UG, or UU. In some aspects, N₁N₂ comprises AG and N₃N₄N₅ comprises any one of the following: AAA, ACA, AGA, AUA, AAG, AGG, ACG, AUG, AAC, ACC, AGC, AUC, AAU, ACU, AGU, AUU, CAA, CCA, CGA, CUA, CAG, CGG, CCG, CUG, CAC, CCC, CGC, CUC, CAU, CCU, CGU, CUU, GAA, GCA, GGA, GUA, GAG, GGG, GCG, GUG, GAC, GCC, GGC, GUC, GAU, GCU, GGU, GUU, UAA, UCA, UGA, UUA, UAG, UGG, UCG, UUG, UAC, UCC, UGC, UUC, UAU, UCU, UGU, or UUU.

In some aspects, a cap proximal sequence comprises N₁ and N₂ of a Cap structure, and a sequence comprising: A₃A₄X₅ (SEQ ID NO: 44; wherein X₅ is A, G, C, or U), where N₁ and N₂ are each independently chosen from: A, C, G, or U. In some aspects, N₁ is A and N₂ is G. In some aspects, X₅ is chosen from A, C, G or U. In some aspects, X₅ is A. In some aspects, X₅ is C. In some aspects, X₅ is G. In some aspects, X₅ is U.

In some aspects, a cap proximal sequence comprises N₁ and N₂ of a Cap structure, and a sequence comprising: C₃A₄X₅ (SEQ ID NO: 45; wherein X₅ is A, G, C, or U), where N₁ and N₂ are each independently chosen from: A, C, G, or U. In some aspects, N₁ is A and N₂ is G. In some aspects, X₅ is chosen from A, C, G or U. In some aspects, X₅ is A. In some aspects, X₅ is C. In some aspects, X₅ is G. In some aspects, X₅ is U.

In some aspects, a cap proximal sequence comprises N₁ and N₂ of a Cap structure, and a sequence comprising X₃Y₄X₅ (SEQ ID NO: 46; wherein X₃ or X₅ are each independently chosen from A, G, C, or U; and Y₄ is not C). In some aspects, N₁ and N₂ are each independently chosen from: A, C, G, or U. In some aspects, N₁ is A and N₂ is G. In some aspects, X₃ and X₅ is each independently chosen from A, C, G or U. In some aspects, X₃ and/or X₅ is A. In some aspects, X₃ and/or X₅ is C. In some aspects, X₃ and/or X₅ is G. In some aspects, X₃ and/or X₅ is U. In some aspects, Y₄ is C. In other aspects, Y₄ is not C. In some aspects, Y₄ is A. In some aspects, Y₄ is G. In other aspects, Y₄ is not G. In some aspects, Y₄ is U.

In some aspects, a cap proximal sequence comprises N₁ and N₂ of a Cap structure, and a sequence comprising A₃C₄A₅ (SEQ ID NO: 47). In some aspects, N₁ and N₂ are each independently chosen from: A, C, G, or U. In some aspects, N₁ is A and N₂ is G.

In some aspects, a cap proximal sequence comprises N₁ and N₂ of a Cap structure, and a sequence comprising A₃U₄G₅ (SEQ ID NO: 48). In some aspects, N₁ and N₂ are each independently chosen from: A, C, G, or U. In some aspects, N₁ is A and N₂ is G.

In some aspects, 1, 2, 3, 4, 5, or more of the foregoing cap proximal sequences can be excluded from the 5′ UTR of the RNA molecules disclosed herein.

In some aspects of the disclosure, a 5′ UTR is a heterologous UTR, e.g., is a UTR found in nature associated with a different ORF. In another aspect, a 5′ UTR is a synthetic UTR, e.g., does not occur in nature. Synthetic UTRs include UTRs that have been mutated or synthesized to improve their properties, e.g., to increase gene expression. In some aspects, the 5′ UTR is functionally linked to the ORF, e.g., associated with the ORF such that it may exert a function, e.g., increasing, enhancing, stabilizing, and/or prolonging protein production from an RNA molecule and/or increasing protein expression and/or total protein production from an RNA molecule, compared to a reference RNA molecule comprising a reference 5′ UTR or an RNA molecule lacking a 5′ UTR. In some aspects, 1, 2, 3, 4, 5, or more of the foregoing 5′ UTR functions can be excluded.

Exemplary 5′ UTRs include 5′ UTRs set forth in Tables 21 and 22 In some aspects, 1, 2, 3, 4, 5, or more of the foregoing 5′ UTR sequences may be excluded from the RNA molecules disclosed herein.

In some aspects, the RNA molecules described herein include one or more 5′UTRs. In one aspect, a DNA encoding a 5′ UTR disclosed herein comprises a sequence having at least, at most, exactly, or between any two of 99%, 98%, 97%, 96%, 95%, 90%, 85%, or 80% identity to any nucleic acid sequence set forth in Table 21. In one aspect, the DNA encoding the 5′ UTR comprises a sequence having at least, at most, exactly, or between any two of 99%, 98%, 97%, 96%, 95%, 90%, 85%, or 80% identity to any nucleic acid sequence selected from the group consisting of SEQ ID NO: 49-279 and 333. In some aspects, the DNA encoding the 5′ UTR described herein comprises a sequence having at least 80% identity to a nucleic acid sequence selected from the group consisting of SEQ ID NOs: 49-279 and 333. In some aspects, the DNA encoding the 5′ UTR described herein comprises a sequence having at least 85% identity to a nucleic acid sequence selected from the group consisting of SEQ ID NOs: 49-279 and 333. In some aspects, the DNA encoding the 5′ UTR described herein comprises a sequence having at least 90% identity to a nucleic acid sequence selected from the group consisting of SEQ ID NOS: 49-279 and 333. In some aspects, the DNA encoding the 5′ UTR described herein comprises a sequence having at least 92% identity to a nucleic acid sequence selected from the group consisting of SEQ ID NOs: 49-279 and 333. In some aspects, the DNA encoding the 5′ UTR described herein comprises a sequence having at least 94% identity to a nucleic acid sequence selected from the group consisting of SEQ ID NOs: 49-279 and 333. In some aspects, the DNA encoding the 5′ UTR described herein comprises a sequence having at least 95% identity to a nucleic acid sequence selected from the group consisting of SEQ ID NOs: 49-279 and 333. In some aspects, the DNA encoding the 5′ UTR described herein comprises a sequence having at least 96% identity to a nucleic acid sequence selected from the group consisting of SEQ ID NOS: 49-279 and 333. In some aspects, the DNA encoding the 5′ UTR described herein comprises a sequence having at least 97% identity to a nucleic acid sequence selected from the group consisting of SEQ ID NOs: 49-279 and 333. In some aspects, the DNA encoding the 5′ UTR described herein comprises a sequence having at least 98% identity to a nucleic acid sequence selected from the group consisting of SEQ ID NOs: 49-279 and 333. In some aspects, the DNA encoding the 5′ UTR described herein comprises a sequence having at least 99% identity to a 15 nucleic acid sequence selected from the group consisting of SEQ ID NOs: 49-279 and 333. In some aspects, the DNA encoding the 5′ UTR described herein comprises a sequence selected from the group consisting of SEQ ID NOs: 49-279 and 333.

In another aspect, an RNA disclosed herein comprises a 5′ UTR having a sequence with at least, at most, exactly, or between any two of 99%, 98%, 97%, 96%, 95%, 90%, 85%, or 80% identity to any nucleic acid sequence set forth in Table 22. In one aspect, the 5′ UTR comprises a sequence having at least, at most, exactly, or between any two of 99%, 98%, 97%, 96%, 95%, 90%, 85%, or 80% identity to a nucleic acid sequence selected from the group consisting of SEQ ID NO: 335-565 and 619. In some aspects, the 5′ UTR comprises a sequence having at least 80% identity to a nucleic acid sequence selected from the group consisting of SEQ ID NOs: 335-565 and 619. In some aspects, the 5′ UTR comprises a sequence having at least 85% identity to a nucleic acid sequence selected from the group consisting of SEQ ID NOs: 335-565 and 619. In some aspects, the 5′ UTR comprises a sequence having at least 90% identity to a nucleic acid sequence selected from the group consisting of SEQ ID NOs: 335-565 and 619. In some aspects, the 5′ UTR comprises a sequence having at least 92% identity to a nucleic acid sequence selected from the group consisting of SEQ ID NOs: 335-565 and 619. In some aspects, the 5′ UTR comprises a sequence having at least 94% identity to a nucleic acid sequence selected from the group consisting of SEQ ID NOs: 335-565 and 619. In some aspects, the 5′ UTR comprises a sequence having at least 95% identity to a nucleic acid sequence selected from the group consisting of SEQ ID NOs: 335-565 and 619. In some aspects, the 5′ UTR described herein comprises a sequence having at least 96% identity to a nucleic acid sequence selected from the group consisting of SEQ ID NOs: 335-565 and 619. In some aspects, the 5′ UTR comprises a sequence having at least 97% identity to a nucleic acid sequence selected from the group consisting of SEQ ID NOs: 335-565 and 619. In some aspects, the 5′ UTR comprises a sequence having at least 98% identity to a nucleic acid sequence selected from the group consisting of SEQ ID NOs: 335-565 and 619. In some aspects, the 5′ UTR comprises a sequence having at least 99% identity to a nucleic acid sequence selected from the group consisting of SEQ ID NOS: 335-565 and 619. In some aspects, the 5′ UTR comprises a sequence selected from the group consisting of SEQ ID NOs: 335-565 and 619.

In one aspect, the 5′ UTR comprises a sequence of any of SEQ ID NO: 49-279, 333, 335-565 or 619, in which the 5′ cap structure is underlined.

In some aspects, 1, 2, 3, or more of the foregoing 5′ UTR sequences may be excluded from the RNA molecules disclosed herein.

In some aspects, an RNA disclosed herein comprises a 3′ UTR. A 3′ UTR, if present, is situated downstream of a protein coding sequence open reading frame, e.g., downstream of the termination codon of a protein-encoding region. A 3′ UTR is typically the part of an mRNA which is located between the protein coding sequence and the poly-A tail of the mRNA. Thus, in some aspects, the 3′ UTR is upstream of the poly-A sequence (if present), e.g. directly adjacent to the poly-A sequence. The 3′ UTR may be involved in regulatory processes including transcript cleavage, stability and polyadenylation, translation, and mRNA localization.

Natural or wild type 3′ UTRs comprise stretches of adenosines and uridines. These AU rich signatures are particularly prevalent in genes with high rates of turnover. Based on their sequence features and functional properties, the AU rich elements (AREs) can be separated into three classes: Class I AREs contain several dispersed copies of an AUUUA motif within U-rich regions. Class II AREs possess two or more overlapping UUAUUUA (U/A) (U/A) nonamers. Class III ARES do not contain an AUUUA motif. Most proteins binding to AREs are known to destabilize the molecule. Accordingly, introduction, removal and/or modification of 3′ UTR AREs can be used to modulate the stability of nucleic acids (e.g., RNA) of the disclosure. When engineering specific nucleic acids, in some aspects, one or more copies of an ARE can be introduced to make RNAs less stable and thereby curtail translation and decrease production of the resultant protein. Likewise, in some aspects, AREs can be identified and removed and/or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection. In some aspects, a 3′ UTR may have one or more AU-rich sequences removed. Alternatively the AU-rich sequences may remain in the 3′ UTR.

A 3′ UTR may also comprise elements, which are not encoded in the template, from which an RNA is transcribed, but which are added after transcription during maturation, e.g. a poly-A tail. A 3′ UTR of the mRNA is not translated into an amino acid sequence. In some aspects, an RNA disclosed herein comprises a 3′ UTR comprising an F element and/or an I element. In some aspects, a 3′ UTR or a proximal sequence thereto comprises a restriction site. In some aspects, a restriction site is a BamHI site. In some aspects, a restriction site is a XhoI site.

In some aspects of the disclosure, a 3′ UTR is a heterologous UTR, e.g., is a UTR found in nature associated with a different ORF. In another aspect, a 3′ UTR is a synthetic UTR, e.g., does not occur in nature. In some aspects, the 3′ UTR is functionally linked to the ORF, e.g., associated with the ORF such that it may exert a function, e.g., increasing, enhancing, stabilizing, and/or prolonging protein production from an RNA molecule and/or increasing protein expression and/or total protein production from an RNA molecule, compared to a reference RNA molecule comprising a reference 3′ UTR or an RNA molecule lacking a 3′ UTR. In some aspects, 1, 2, 3, 4, 5, or more of the foregoing 3′ UTR functions may be excluded.

Exemplary 3′ UTRs include 3′ UTRs set forth in Tables 21 and 22 In some aspects, the sequence UUUGAAUU is used. In some aspects, 1, 2, 3, 4, 5, or more of the foregoing 3′ UTR sequences may be excluded from the RNA molecules disclosed herein.

In some aspects, the RNA molecules described herein include one or more 3′UTRs.

In one aspect, a DNA encoding a 3′ UTR disclosed herein comprises a sequence having at least, at most, exactly, or between any two of 99%, 98%, 97%, 96%, 95%, 90%, 85%, or 80% identity to any nucleic acid sequence set forth in Table 21. In one aspect, the DNA encoding the 3′ UTR comprises a sequence having at least, at most, exactly, or between any two of 99%, 98%, 97%, 96%, 95%, 90%, 85%, or 80% identity to any nucleic acid sequence selected from the group consisting of SEQ ID NO: 280-332 and 334. In some aspects, the DNA encoding the 3′ UTR described herein comprises a sequence having at least 80% identity to a nucleic acid sequence selected from the group consisting of SEQ ID NO: 280-332 and 334. In some aspects, the DNA encoding the 3′ UTR described herein comprises a sequence having at least 85% identity to a nucleic acid sequence selected from the group consisting of SEQ ID NO: 280-332 and 334. In some aspects, the DNA encoding the 3′ UTR described herein comprises a sequence having at least 90% identity to a nucleic acid sequence selected from the group consisting of SEQ ID NO: 280-332 and 334. In some aspects, the DNA encoding the 3′ UTR described herein comprises a sequence having at least 92% identity to a nucleic acid sequence selected from the group consisting of SEQ ID NO: 280-332 and 334. In some aspects, the DNA encoding the 3′ UTR described herein comprises a sequence having at least 94% identity to a nucleic acid sequence selected from the group consisting of SEQ ID NO: 280-332 and 334. In some aspects, the DNA encoding the 3′ UTR described herein comprises a sequence having at least 95% identity to a nucleic acid sequence selected from the group consisting of SEQ ID NO: 280-332 and 334. In some aspects, the DNA encoding the 3′ UTR described herein comprises a sequence having at least 96% identity to a nucleic acid sequence selected from the group consisting of SEQ ID NO: 280-332 and 334. In some aspects, the DNA encoding the 3′ UTR described herein comprises a sequence having at least 97% identity to a nucleic acid sequence selected from the group consisting of SEQ ID NO: 280-332 and 334. In some aspects, the DNA encoding the 3′ UTR described herein comprises a sequence having at least 98% identity to a nucleic acid sequence selected from the group consisting of SEQ ID NO: 280-332 and 334. In some aspects, the DNA encoding the 3′ UTR described herein comprises a sequence having at least 99% identity to a nucleic acid sequence selected from the group consisting of SEQ ID NO: 280-332 and 334. In some aspects, the DNA encoding the 3′ UTR described herein comprises a sequence selected from the group consisting of SEQ ID NO: 280-332 and 334.

In another aspect, an RNA disclosed herein comprises a 3′ UTR having a sequence with at least, at most, exactly, or between any two of 99%, 98%, 97%, 96%, 95%, 90%, 85%, or 80% identity to any nucleic acid sequence set forth in Table 22. In one aspect, the 3′ UTR comprises a sequence having at least, at most, exactly, or between any two of 99%, 98%, 97%, 96%, 95%, 90%, 85%, or 80% identity to a nucleic acid sequence selected from the group consisting of SEQ ID NO: 566-618 and 620. In some aspects, the 3′ UTR comprises a sequence having at least 80% identity to a nucleic acid sequence selected from the group consisting of SEQ ID NOs: 566-618 and 620. In some aspects, the 3′ UTR comprises a sequence having at least 85% identity to a nucleic acid sequence selected from the group consisting of SEQ ID NOs: 566-618 and 620. In some aspects, the 3′ UTR comprises a sequence having at least 90% identity to a nucleic acid sequence selected from the group consisting of SEQ ID NOs: 566-618 and 620. In some aspects, the 3′ UTR comprises a sequence having at least 92% identity to a nucleic acid sequence selected from the group consisting of SEQ ID NOs: 566-618 and 620. In some aspects, the 3′ UTR comprises a sequence having at least 94% identity to a nucleic acid sequence selected from the group consisting of SEQ ID NOs: 566-618 and 620. In some aspects, the 3′ UTR comprises a sequence having at least 95% identity to a nucleic acid sequence selected from the group consisting of SEQ ID NOs: 566-618 and 620. In some aspects, the 3′ UTR described herein comprises a sequence having at least 96% identity to a nucleic acid sequence selected from the group consisting of SEQ ID NOs: 566-618 and 620. In some aspects, the 3′ UTR comprises a sequence having at least 97% identity to a nucleic acid sequence selected from the group consisting of SEQ ID NOs: 566-618 and 620. In some aspects, the 3′ UTR comprises a sequence having at least 98% identity to a nucleic acid sequence selected from the group consisting of SEQ ID NOs: 566-618 and 620. In some aspects, the 3′ UTR comprises a sequence having at least 99% identity to a nucleic acid sequence selected from the group consisting of SEQ ID NOS: 566-618 and 620. In some aspects, the 3′ UTR comprises a sequence selected from the group consisting of SEQ ID NOs: 566-618 and 620.

In some aspects, the third nucleotide of any one of SEQ ID NOs: 49-620 is replaced with an adenine (A). For example, 5UTR_14 (SEQ ID NO:49) would begin “AGA” instead of “AGG.” In some aspects, the third nucleotide of any one of SEQ ID NOs: 49-620 is replaced with a cytosine (C). For example, 5UTR_14 (SEQ ID NO:49) would begin “AGC” instead of “AGG.”

In some aspects, the RNA molecules described herein do not consist of SEQ ID NO: 63 and SEQ ID NO: 232.

TABLE 21
DNA Sequences for UTRs described herein

| Name | SEQ ID NO: |
|---|---|
| 5UTR_14 | 49 |
| 5UTR_15 (BMD2) | 50 |
| 5UTR_16 (BMD3) | 51 |
| 5UTR_70 | 52 |
| 5UTR_73 | 53 |
| 5UTR_91 | 54 |
| 5UTR_105 | 55 |
| 5UTR_109 | 56 |
| 5UTR_123 | 57 |
| 5UTR_260 | 58 |
| 5UTR_261 | 59 |
| 5UTR_476 | 60 |
| 5UTR_477 | 61 |
| 5UTR_482 | 62 |
| 5UTR_484 | 63 |
| 5UTR_487 | 64 |
| 5UTR_488 | 65 |
| 5UTR_489 | 66 |
| 5UTR_566 | 67 |
| SEED1 | 68 |
| SEED2 | 69 |
| 5UTR-105_variant 0012 | 70 |
| 5UTR_579 | 71 |
| 5UTR-105_variant 0014 | 72 |
| 5UTR_2 hHBB reference | 73 |
| hHBB ss-opt | 74 |
| SEED4 | 75 |
| 5UTR_263 | 76 |
| 5UTR_271 | 77 |
| 5UTR_273 | 78 |
| 5UTR_284 | 79 |
| 5UTR_305 | 80 |
| 5UTR_382 | 81 |
| 5UTR_509 | 82 |
| 5UTR_515 | 83 |
| 5UTR_552 | 84 |
| 5UTR_556 | 85 |
| 5UTR_562 | 86 |
| 5UTR_563 | 87 |
| 5UTR_576 | 88 |
| 5UTR_577 | 89 |
| 5UTR_444 | 90 |
| 5UTR_3 (hActB reference) | 91 |
| 5UTR_581 | 92 |
| 5UTR_595 | 93 |
| 5UTR_582 | 94 |
| 5UTR_598 | 95 |
| 5UTR_586 | 96 |
| 5UTR_590 | 97 |
| 5UTR_583 | 98 |
| 5UTR_599 | 99 |
| 5UTR_584 | 100 |
| 5UTR_593 | 101 |
| 5UTR_588 | 102 |
| 5UTR_594 | 103 |
| 5UTR_587 | 104 |
| 5UTR_597 | 105 |
| 5UTR_596 | 106 |
| 5UTR_592 | 107 |
| 5UTR_591 | 108 |
| 5UTR_589 | 109 |
| 5UTR_585 | 110 |
| 5UTR_1 (WHO reference) | 111 |
| 5UTR_2 | 112 |
| 5UTR_42 | 113 |
| 5UTR_44 | 114 |
| 5UTR_45 | 115 |
| 5UTR_46 | 116 |
| 5UTR_47 | 117 |
| 5UTR_48 | 118 |
| 5UTR_49 | 119 |
| 5UTR_50 | 120 |
| 5UTR_51 | 121 |
| 5UTR_52 | 122 |
| 5UTR_53 | 123 |
| 5UTR_54 | 124 |
| 5UTR_55 | 125 |
| 5UTR_56 | 126 |
| 5UTR_58 | 127 |
| 5UTR_59 | 128 |
| 5UTR_60 | 129 |
| 5UTR_61 | 130 |
| 5UTR_62 | 131 |
| 5UTR_63 | 132 |
| 5UTR_64 | 133 |
| 5UTR_65 | 134 |
| 5UTR_66 | 135 |
| 5UTR_67 | 136 |
| 5UTR_68 | 137 |
| 5UTR_69 | 138 |
| 5UTR_71 | 139 |
| 5UTR_72 | 140 |
| 5UTR_74 | 141 |
| 5UTR_75 | 142 |
| 5UTR_76 | 143 |
| 5UTR_77 | 144 |
| 5UTR_78 | 145 |
| 5UTR_79 | 146 |
| 5UTR_80 | 147 |
| 5UTR_81 | 148 |
| 5UTR_82 | 149 |
| 5UTR_83 | 150 |
| 5UTR_84 | 151 |
| 5UTR_85 | 152 |
| 5UTR_86 | 153 |
| 5UTR_88 | 154 |
| 5UTR_89 | 155 |
| 5UTR_90 | 156 |
| 5UTR_92 | 157 |
| 5UTR_93 | 158 |
| 5UTR_95 | 159 |
| 5UTR_96 | 160 |
| 5UTR_97 | 161 |
| 5UTR_98 | 162 |
| 5UTR_100 | 163 |
| 5UTR_102 | 164 |
| 5UTR_103 | 165 |
| 5UTR_106 | 166 |
| 5UTR_107 | 167 |
| 5UTR_108 | 168 |
| 5UTR_110 | 169 |
| 5UTR_111 | 170 |
| 5UTR_113 | 171 |
| 5UTR_114 | 172 |
| 5UTR_116 | 173 |
| 5UTR_117 | 174 |
| 5UTR_119 | 175 |
| 5UTR_120 | 176 |
| 5UTR_122 | 177 |
| 5UTR_124 | 178 |
| 5UTR_125 | 179 |
| 5UTR_126 | 180 |
| 5UTR_127 | 181 |
| 5UTR_128 | 182 |
| 5UTR_129 | 183 |
| 5UTR_131 | 184 |
| 5UTR_132 | 185 |
| 5UTR_133 | 186 |
| 5UTR_135 | 187 |
| 5UTR_136 | 188 |
| 5UTR_137 | 189 |
| 5UTR_138 | 190 |
| 5UTR_139 | 191 |
| 5UTR_140 | 192 |
| 5UTR_141 | 193 |
| 5UTR_142 | 194 |
| 5UTR_144 | 195 |
| 5UTR_145 | 196 |
| 5UTR_146 | 197 |
| 5UTR_147 | 198 |
| 5UTR_148 | 199 |
| 5UTR_149 | 200 |
| 5UTR_150 | 201 |
| 5UTR_151 | 202 |
| 5UTR_152 | 203 |
| 5UTR_153 | 204 |
| 5UTR_155 | 205 |
| 5UTR_157 | 206 |
| 5UTR_158 | 207 |
| 5UTR_159 | 208 |
| 5UTR_160 | 209 |
| 5UTR_161 | 210 |
| 5UTR_163 | 211 |
| 5UTR_164 | 212 |
| 5UTR_165 | 213 |
| 5UTR_166 | 214 |
| 5UTR_168 | 215 |
| 5UTR_169 | 216 |
| 5UTR_171 | 217 |
| 5UTR_172 | 218 |
| 5UTR_173 | 219 |
| 5UTR_174 | 220 |
| 5UTR_175 | 221 |
| 5UTR_176 | 222 |
| 5UTR_177 | 223 |
| 5UTR_178 | 224 |
| 5UTR_179 | 225 |
| 5UTR_180 | 226 |
| 5UTR_181 | 227 |
| 5UTR_184 | 228 |
| 5UTR_186 | 229 |
| 5UTR_187 | 230 |
| 5UTR_188 | 231 |
| 5UTR_190 | 232 |
| 5UTR_192 | 233 |
| 5UTR_193 | 234 |
| 5UTR_194 | 235 |
| 5UTR_195 | 236 |
| 5UTR_196 | 237 |
| 5UTR_197 | 238 |
| 5UTR_199 | 239 |
| 5UTR_200 | 240 |
| 5UTR_201 | 241 |
| 5UTR_202 | 242 |
| 5UTR_203 | 243 |
| 5UTR_204 | 244 |
| 5UTR_205 | 245 |
| 5UTR_206 | 246 |
| 5UTR_207 | 247 |
| 5UTR_209 | 248 |
| 5UTR_210 | 249 |
| 5UTR_212 | 250 |
| 5UTR_213 | 251 |
| 5UTR_214 | 252 |
| 5UTR_217 | 253 |
| 5UTR_223 | 254 |
| 5UTR_224 | 255 |
| 5UTR_225 | 256 |
| 5UTR_226 | 257 |
| 5UTR_228 | 258 |
| 5UTR_229 | 259 |
| 5UTR_232 | 260 |
| 5UTR_233 | 261 |
| 5UTR_235 | 262 |
| 5UTR_236 | 263 |
| 5UTR_238 | 264 |
| 5UTR_239 | 265 |
| 5UTR_240 | 266 |
| 5UTR_241 | 267 |
| 5UTR_243 | 268 |
| 5UTR_244 | 269 |
| 5UTR_245 | 270 |
| 5UTR_246 | 271 |
| 5UTR_249 | 272 |
| 5UTR_251 | 273 |
| 5UTR_253 | 274 |
| 5UTR_254 | 275 |
| 5UTR_255 | 276 |
| 5UTR_257 | 277 |
| 5UTR_259 | 278 |
| 5UTR_262 | 279 |
| 3UTR_1 (WHO reference) | 280 |
| 3UTR_2 (hHBB reference) | 281 |
| 3UTR_7 (C3PO reference) | 282 |
| 3UTR_8 | 283 |
| 3UTR_10 | 284 |
| 3UTR_11 | 285 |
| 3UTR_16 | 286 |
| 3UTR_17 | 287 |
| 3UTR_18 | 288 |
| 3UTR_19 | 289 |
| 3UTR_21 | 290 |
| 3UTR_22 | 291 |
| 3UTR_24 | 292 |
| 3UTR_25 | 293 |
| 3UTR_27 | 294 |
| 3UTR_28 | 295 |
| 3UTR_29 | 296 |
| 3UTR_30 | 297 |
| 3UTR_31 | 298 |
| 3UTR_32 | 299 |
| 3UTR_33 | 300 |
| 3UTR_34 | 301 |
| 3UTR_35 | 302 |
| 3UTR_36 | 303 |
| 3UTR_37 | 304 |
| 3UTR_38 | 305 |
| 3UTR_39 | 306 |
| 3UTR_40 | 307 |
| 3UTR_43 | 308 |
| 3UTR_44 | 309 |
| 3UTR_45 | 310 |
| 3UTR_46 | 311 |
| 3UTR_47 | 312 |
| 3UTR_48 | 313 |
| 3UTR_49 | 314 |
| 3UTR_50 | 315 |
| 3UTR_51 | 316 |
| 3UTR_52 | 317 |
| 3UTR_53 | 318 |
| 3UTR_55 | 319 |
| 3UTR_56 | 320 |
| 3UTR_57 | 321 |
| 3UTR_58 | 322 |
| 3UTR_59 | 323 |
| 3UTR_61 | 324 |
| 3UTR_62 (AES) | 325 |
| 3UTR_63 | 326 |
| 3UTR_65 | 327 |
| 3UTR_66 | 328 |
| 3UTR_67 | 329 |
| 3UTR_68 | 330 |
| 3UTR_70 | 331 |
| 3UTR_71 | 332 |
| 5UTR_5 (NCA7D) | 333 |
| 3UTR_5 TPRKB-derived | 334 |

TABLE 22
RNA Sequences for UTRs described herein

| Name | SEQ ID NO: |
|---|---|
| 5UTR_14 | 335 |
| 5UTR_15 (BMD2) | 336 |
| 5UTR_16 (BMD3) | 337 |
| 5UTR_70 | 338 |
| 5UTR_73 | 339 |
| 5UTR_91 | 340 |
| 5UTR_105 | 341 |
| 5UTR_109 | 342 |
| 5UTR_123 | 343 |
| 5UTR_260 | 344 |
| 5UTR_261 | 345 |
| 5UTR_476 | 346 |
| 5UTR_477 | 347 |
| 5UTR_482 | 348 |
| 5UTR_484 | 349 |
| 5UTR_487 | 350 |
| 5UTR_488 | 351 |
| 5UTR_489 | 352 |
| 5UTR_566 | 353 |
| SEED1 | 354 |
| SEED2 | 355 |
| 5UTR-105_variant 0012 | 356 |
| 5UTR_579 | 357 |
| 5UTR-105_variant 0014 | 358 |
| 5UTR_2 hHBB reference | 359 |
| hHBB ss-opt | 360 |
| SEED4 | 361 |
| 5UTR_263 | 362 |
| 5UTR_271 | 363 |
| 5UTR_273 | 364 |
| 5UTR_284 | 365 |
| 5UTR_305 | 366 |
| 5UTR_382 | 367 |
| 5UTR_509 | 368 |
| 5UTR_515 | 369 |
| 5UTR_552 | 370 |
| 5UTR_556 | 371 |
| 5UTR_562 | 372 |
| 5UTR_563 | 373 |
| 5UTR_576 | 374 |
| 5UTR_577 | 375 |
| 5UTR_444 | 376 |
| 5UTR_3 (hActB reference) | 377 |
| 5UTR_581 | 378 |
| 5UTR_595 | 379 |
| 5UTR_582 | 380 |
| 5UTR_598 | 381 |
| 5UTR_586 | 382 |
| 5UTR_590 | 383 |
| 5UTR_583 | 384 |
| 5UTR_599 | 385 |
| 5UTR_584 | 386 |
| 5UTR_593 | 387 |
| 5UTR_588 | 388 |
| 5UTR_594 | 389 |
| 5UTR_587 | 390 |
| 5UTR_597 | 391 |
| 5UTR_596 | 392 |
| 5UTR_592 | 393 |
| 5UTR_591 | 394 |
| 5UTR_589 | 395 |
| 5UTR_585 | 396 |
| 5UTR_1 (WHO reference) | 397 |
| | 398 |
| 5UTR_42 | 399 |
| 5UTR_44 | 400 |
| 5UTR_45 | 401 |
| 5UTR_46 | 402 |
| 5UTR_47 | 403 |
| 5UTR_48 | 404 |
| 5UTR_49 | 405 |
| 5UTR_50 | 406 |
| 5UTR_51 | 407 |
| 5UTR_52 | 408 |
| 5UTR_53 | 409 |
| 5UTR_54 | 410 |
| 5UTR_55 | 411 |
| 5UTR_56 | 412 |
| 5UTR_58 | 413 |
| 5UTR_59 | 414 |
| 5UTR_60 | 415 |
| 5UTR_61 | 416 |
| 5UTR_62 | 417 |
| 5UTR_63 | 418 |
| 5UTR_64 | 419 |
| 5UTR_65 | 420 |
| 5UTR_66 | 421 |
| 5UTR_67 | 422 |
| 5UTR_68 | 423 |
| 5UTR_69 | 424 |
| 5UTR_71 | 425 |
| 5UTR_72 | 426 |
| 5UTR_74 | 427 |
| 5UTR_75 | 428 |
| 5UTR_76 | 429 |
| 5UTR_77 | 430 |
| 5UTR_78 | 431 |
| 5UTR_79 | 432 |
| 5UTR_80 | 433 |
| 5UTR_81 | 434 |
| 5UTR_82 | 435 |
| 5UTR_83 | 436 |
| 5UTR_84 | 437 |
| 5UTR_85 | 438 |
| 5UTR_86 | 439 |
| 5UTR_88 | 440 |
| 5UTR_89 | 441 |
| 5UTR_90 | 442 |
| 5UTR_92 | 443 |
| 5UTR_93 | 444 |
| 5UTR_95 | 445 |
| 5UTR_96 | 446 |
| 5UTR_97 | 447 |
| 5UTR_98 | 448 |
| 5UTR_100 | 449 |
| 5UTR_102 | 450 |
| 5UTR_103 | 451 |
| 5UTR_106 | 452 |
| 5UTR_107 | 453 |
| 5UTR_108 | 454 |
| 5UTR_110 | 455 |
| 5UTR_111 | 456 |
| 5UTR_113 | 457 |
| 5UTR_114 | 458 |
| 5UTR_116 | 459 |
| 5UTR_117 | 460 |
| 5UTR_119 | 461 |
| 5UTR_120 | 462 |
| 5UTR_122 | 463 |
| 5UTR_124 | 464 |
| 5UTR_125 | 465 |
| 5UTR_126 | 466 |
| 5UTR_127 | 467 |
| 5UTR_128 | 468 |
| 5UTR_129 | 469 |
| 5UTR_131 | 470 |
| 5UTR_132 | 471 |
| 5UTR_133 | 472 |
| 5UTR_135 | 473 |
| 5UTR_136 | 474 |
| 5UTR_137 | 475 |
| 5UTR_138 | 476 |
| 5UTR_139 | 477 |
| 5UTR_140 | 478 |
| 5UTR_141 | 479 |
| 5UTR_142 | 480 |
| 5UTR_144 | 481 |
| 5UTR_145 | 482 |
| 5UTR_146 | 483 |
| 5UTR_147 | 484 |
| 5UTR_148 | 485 |
| 5UTR_149 | 486 |
| 5UTR_150 | 487 |
| 5UTR_151 | 488 |
| 5UTR_152 | 489 |
| 5UTR_153 | 490 |
| 5UTR_155 | 491 |
| 5UTR_157 | 492 |
| 5UTR_158 | 493 |
| 5UTR_159 | 494 |
| 5UTR_160 | 495 |
| 5UTR_161 | 496 |
| 5UTR_163 | 497 |
| 5UTR_164 | 498 |
| 5UTR_165 | 499 |
| 5UTR_166 | 500 |
| 5UTR_168 | 501 |
| 5UTR_169 | 502 |
| 5UTR_171 | 503 |
| 5UTR_172 | 504 |
| 5UTR_173 | 505 |
| 5UTR_174 | 506 |
| 5UTR_175 | 507 |
| 5UTR_176 | 508 |
| 5UTR_177 | 509 |
| 5UTR_178 | 510 |
| 5UTR_179 | 511 |
| 5UTR_180 | 512 |
| 5UTR_181 | 513 |
| 5UTR_184 | 514 |
| 5UTR_186 | 515 |
| 5UTR_187 | 516 |
| 5UTR_188 | 517 |
| 5UTR_190 | 518 |
| 5UTR_192 | 519 |
| 5UTR_193 | 520 |
| 5UTR_194 | 521 |
| 5UTR_195 | 522 |
| 5UTR_196 | 523 |
| 5UTR_197 | 524 |
| 5UTR_199 | 525 |
| 5UTR_200 | 526 |
| 5UTR_201 | 527 |
| 5UTR_202 | 528 |
| 5UTR_203 | 529 |
| 5UTR_204 | 530 |
| 5UTR_205 | 531 |
| 5UTR_206 | 532 |
| 5UTR_207 | 533 |
| 5UTR_209 | 534 |
| 5UTR_210 | 535 |
| 5UTR_212 | 536 |
| 5UTR_213 | 537 |
| 5UTR_214 | 538 |
| 5UTR_217 | 539 |
| 5UTR_223 | 540 |
| 5UTR_224 | 541 |
| 5UTR_225 | 542 |
| 5UTR_226 | 543 |
| 5UTR_228 | 544 |
| 5UTR_229 | 545 |
| 5UTR_232 | 546 |
| 5UTR_233 | 547 |
| 5UTR_235 | 548 |
| 5UTR_236 | 549 |
| 5UTR_238 | 550 |
| 5UTR_239 | 551 |
| 5UTR_240 | 552 |
| 5UTR_241 | 553 |
| 5UTR_243 | 554 |
| 5UTR_244 | 555 |
| 5UTR_245 | 556 |
| 5UTR_246 | 557 |
| 5UTR_249 | 558 |
| 5UTR_251 | 559 |
| 5UTR_253 | 560 |
| 5UTR_254 | 561 |
| 5UTR_255 | 562 |
| 5UTR_257 | 563 |
| 5UTR_259 | 564 |
| 5UTR_262 | 565 |
| 3UTR_1 (WHO reference) | 566 |
| 3UTR_2 (hHBB reference) | 567 |
| 3UTR_7 (C3PO reference) | 568 |
| 3UTR_8 | 569 |
| 3UTR_10 | 570 |
| 3UTR_11 | 571 |
| 3UTR_16 | 572 |
| 3UTR_17 | 573 |
| 3UTR_18 | 574 |
| 3UTR_19 | 575 |
| 3UTR_21 | 576 |
| 3UTR_22 | 577 |
| 3UTR_24 | 578 |
| 3UTR_25 | 579 |
| 3UTR_27 | 580 |
| 3UTR_28 | 581 |
| 3UTR_29 | 582 |
| 3UTR_30 | 583 |
| 3UTR_31 | 584 |
| 3UTR_32 | 585 |
| 3UTR_33 | 586 |
| 3UTR_34 | 587 |
| 3UTR_35 | 588 |
| 3UTR_36 | 589 |
| 3UTR_37 | 590 |
| 3UTR_38 | 591 |
| 3UTR_39 | 592 |
| 3UTR_40 | 593 |
| 3UTR_43 | 594 |
| 3UTR_44 | 595 |
| 3UTR_45 | 596 |
| 3UTR_46 | 597 |
| 3UTR_47 | 598 |
| 3UTR_48 | 599 |
| 3UTR_49 | 600 |
| 3UTR_50 | 601 |
| 3UTR_51 | 602 |
| 3UTR_52 | 603 |
| 3UTR_53 | 604 |
| 3UTR_55 | 605 |
| 3UTR_56 | 606 |
| 3UTR_57 | 607 |
| 3UTR_58 | 608 |
| 3UTR_59 | 609 |
| 3UTR_61 | 610 |
| 3UTR_62 (AES) | 611 |
| 3UTR_63 | 612 |
| 3UTR_65 | 613 |
| 3UTR_66 | 614 |
| 3UTR_67 | 615 |
| 3UTR_68 | 616 |
| 3UTR_70 | 617 |
| 3UTR_71 | 618 |
| 5UTR_5 (NCA7D) | 619 |
| 3UTR_5 UPRKB-derived | 620 |

### D. Open Reading Frame (ORF)

The 5′ and 3′ UTRs may be operably linked to an open reading frame (ORF), which may be a sequence of codons that is capable of being translated into a polypeptide of interest. An open reading frame may be a sequence of several DNA or RNA nucleotide triplets, which may be translated into a peptide or protein. An ORF may begin with a start codon, e.g., a combination of three subsequent nucleotides coding usually for the amino acid methionine (ATG or AUG), at its 5′ end and a subsequent region, which usually exhibits a length which is a multiple of 3 nucleotides. An open reading frame may terminate with at least one stop codon, including but not limited to TAA, TAG, TGA or UAA, UAG or UGA, or any combination thereof. In some aspects, an open reading frame may terminate with one, two, three, four or more stop codons, including but not limited to TAATAA (SEQ ID NO: 25), TAATAG (SEQ ID NO: 26), TAATGA (SEQ ID NO: 27), TAGTGA (SEQ ID NO: 28), TAGTAA (SEQ ID NO: 29), TAGTAG (SEQ ID NO: 30), TGATGA (SEQ ID NO: 31), TGATAG (SEQ ID NO: 32), TGATAA (SEQ ID NO: 33) or UAAUAA (SEQ ID NO: 34), UAAUAG (SEQ ID NO: 35), UAAUGA (SEQ ID NO: 36), UAGUGA (SEQ ID NO: 37), UAGUAA (SEQ ID NO:38), UAGUAG (SEQ ID NO: 39), UGAUGA (SEQ ID NO: 40), UGAUAG (SEQ ID NO: 41), UGAUAA (SEQ ID NO: 42), or any combination thereof. An open reading frame may be isolated or it may be incorporated in a longer nucleic acid sequence, e.g. in a vector or an mRNA. An open reading frame may also be termed “(protein) coding region” or “coding sequence”.

As stated herein, the RNA molecule may include one (monocistronic), two (bicistronic) or more (multicistronic) open reading frames.

In some aspects, the ORF encodes a non-structural viral gene. In some aspects, the ORF further includes one or more subgenomic promoters. In some aspects, the RNA molecule includes a subgenomic promoter operably linked to the ORF. In some aspects, a first RNA molecule does not include an ORF encoding any polypeptide of interest, whereas a second RNA molecule includes an ORF encoding a polypeptide of interest. In some aspects, the first RNA molecule does not include a subgenomic promoter.

The present disclosure provides for an RNA molecule comprising at least one open reading frame encoding a respiratory syncytial virus (RSV) polypeptide. In some aspects, an RNA molecule comprising at least one open reading frame encoding a RSV F protein. In a preferred aspect, an RNA molecule comprising at least one open reading frame encoding a respiratory syncytial virus (RSV) prefusion F protein (preF) polypeptide.

### E. Genes of Interest

In some aspects, the RNA molecules include a coding region for a gene of interest. In some aspects, a gene of interest is or comprises an antigenic polypeptide or an immunogenic variant or an immunogenic fragment thereof. In some aspects, an antigenic polypeptide comprises one epitope from an antigen. In some aspects, an antigenic polypeptide comprises a plurality of distinct epitopes from an antigen. In some aspects, an antigenic polypeptide comprising a plurality of distinct epitopes from an antigen is polyepitopic. In some aspects, an antigenic polypeptide comprises: an antigenic polypeptide from an allergen, a viral antigenic polypeptide, a bacterial antigenic polypeptide, a fungal antigenic polypeptide, a parasitic antigenic polypeptide, an antigenic polypeptide from an infectious agent, an antigenic polypeptide from a pathogen, a tumor antigenic polypeptide, or a self-antigenic polypeptide. In some aspects, 1, 2, 3, 4, 5, or more of the foregoing antigenic polypeptides may be excluded.

The term “antigen” may refer to a substance, which is capable of being recognized by the immune system, e.g., by the adaptive immune system, and which is capable of eliciting an antigen-specific immune response, e.g., by formation of antibodies and/or antigen-specific T cells as part of an adaptive immune response. An antigen may be or may comprise a peptide or protein, which may be presented by the MHC to T cells. An antigen may be the product of translation of a provided nucleic acid molecule, e.g., an RNA molecule comprising at least one coding sequence as described herein. In addition, fragments, variants and derivatives of an antigen, such as a peptide or a protein, comprising at least one epitope are understood as antigens.

In some aspects, an RNA encoding a gene of interest, e.g., an antigen, is expressed in cells of a subject treated to provide a gene of interest, e.g., an antigen. In some aspects, the RNA is transiently expressed in cells of the subject. In some aspects, expression of a gene of interest, e.g., an antigen, is at the cell surface. In some aspects, a gene of interest, e.g., an antigen, is expressed and presented in the context of MHC. In some aspects, expression of a gene of interest, e.g., an antigen, is into the extracellular space, e.g., the antigen is secreted.

In some aspects, the RNA molecules include a coding region for a gene of interest, e.g., an antigen. In some aspects, the RNA molecules include a coding region for a gene of interest, e.g., an antigen, that is derived from a pathogen associated with an infectious disease. In some aspects, the RNA molecules include a coding region for a gene of interest, e.g., an antigen, that is derived from RSV, PIV1, PIV3 or hMPV.

In some aspects, the RNA molecule encodes a RSV preF protein or a fragment or a variant thereof.

In some aspects, the RNA molecule encodes a PIV1 preF protein or a fragment or a variant thereof.

In some aspects, the RNA molecule encodes a PIV3 preF protein or a fragment or a variant thereof.

In some aspects, the RNA molecule encodes a hMPV preF protein or a fragment or a variant thereof.

In some aspects, an RNA polynucleotide described herein or a composition or medical preparation comprising the same comprises a nucleotide sequence disclosed herein. In some aspects, an RNA polynucleotide comprises a sequence having at least 80% identity to a nucleotide sequence disclosed herein. In some aspects, an RNA polynucleotide comprises a sequence encoding a polypeptide having at least 80% identity to a polypeptide sequence disclosed herein. In some aspects, an RNA polynucleotide described herein or a composition or medical preparation comprising the same is transcribed by a DNA template. In some aspects, a DNA template used to transcribe an RNA polynucleotide described herein comprises a sequence complementary to an RNA polynucleotide. In some aspects, a gene of interest described herein is encoded by an RNA polynucleotide described herein comprising a nucleotide sequence disclosed herein. In some aspects, an RNA polynucleotide encodes a polypeptide having at least 80% identity to a polypeptide sequence disclosed herein. In some aspects, a polypeptide described herein is encoded by an RNA polynucleotide transcribed by a DNA template comprising a sequence complementary to an RNA polynucleotide.

### F. Poly-A Tail

In some aspects, RNA molecules disclosed herein comprise a poly-adenylate (poly-A) sequence, e.g., as described herein. In some aspects, a poly-A sequence is situated downstream of a 3′ UTR, e.g., adjacent to a 3′ UTR. A “poly-A tail” or “poly-A sequence” refers to a stretch of consecutive adenine residues, e.g., of up to or up to about 400 adenosine nucleotides, e.g., from or from about 20 to about 400, preferably from or from about 50 to about 400, more preferably from or from about 50 to about 300, even more preferably from or from about 50 to about 250, most preferably from or from about 60 to about 250 adenosine nucleotides, which may be attached to the 3′ end of the RNA molecule. Poly-A sequences are known to those of skill in the art and may follow the 3′ UTR in the RNA molecules described herein. The poly-A tail may increase the stability, half-life, and/or translational efficiency of the RNA molecule.

After cleavage, most pre-mRNAs, with exceptions that include replication-dependent histone transcripts that terminate with a histone stem-loop instead of a poly-A sequence, acquire a polyadenylated tail. In this context, 3′-end processing is a nuclear co-transcriptional process that promotes transport of mRNAs from the nucleus to the cytoplasm and affects the stability and the translation of mRNAs. Formation of this 3′ end occurs in a two-step reaction directed by the cleavage/polyadenylation machinery and depends on the presence of two sequence elements in mRNA precursors (pre-mRNAs); a hexanucleotide polyadenylation signal and a downstream G/U-rich sequence. In a first step, pre-mRNAs are cleaved between these two elements to a free 3′ hydroxyl. In a second step, the newly formed 3′ end is extended by polyadenylation or addition of a poly-A sequence.

Polyadenylation refers to the addition of a poly-A sequence to an RNA molecule, e.g., to a premature mRNA. Polyadenylation may be induced by a so-called polyadenylation signal. This signal may be located within a stretch of nucleotides close to or at the 3′-end of an RNA molecule to be polyadenylated. A polyadenylation signal may also be comprised by the 3′ UTR of the artificial nucleic acid molecule. A polyadenylation signal typically comprises a hexamer consisting of adenine and uracil/thymine nucleotides, preferably the hexamer sequence AAUAAA, though other sequences, preferably hexamer sequences, are also conceivable. Polyadenylation typically occurs during processing of a pre-mRNA (also called premature-mRNA). Typically, RNA maturation (from pre-mRNA to mature mRNA) comprises the step of polyadenylation. Poly-A tailing of in vitro transcribed mRNA can be achieved using various approaches including, but not limited to, cloning of a poly-T tract into the DNA template or by post-transcriptional addition using poly-A polymerase. The term may relate to polyadenylation of RNA as a cellular process or to polyadenylation carried out by enzymatic reaction in vitro with a suitable enzyme, such as E. coli poly-A polymerase, or by chemical synthesis.

RNA molecules disclosed herein may have a poly-A sequence attached to the free 3′-end of the RNA by a template-independent RNA polymerase after transcription or a poly-A sequence encoded by DNA and transcribed by a template-dependent RNA polymerase. In some aspects, a poly-A sequence is attached during RNA transcription, e.g., during preparation of in vitro transcribed RNA, based on a DNA template comprising repeated dT nucleotides (deoxythymidylate) in the strand complementary to the coding strand.

The DNA sequence encoding a poly-A sequence (coding strand) is referred to as poly-A cassette. In some aspects, the poly-A cassette present in the coding strand of DNA essentially consists of dA nucleotides, but is interrupted by a random sequence of the four nucleotides (dA, dC, dG, and dT). Such a random sequence may be at least, at most, exactly, or between (inclusive or exclusive) any two of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. Such a cassette is disclosed in, e.g., WO 2016/005324 A1, hereby incorporated by reference. Any poly-A cassette disclosed in WO 2016/005324 A1 may be used in the present disclosure. A poly-A cassette that essentially consists of dA nucleotides, but is interrupted by a random sequence having an equal distribution of the four nucleotides (dA, dC, dG, dT) and having a length of e.g., 5 to 50 nucleotides, shows, on a DNA level, constant propagation of plasmid DNA in E. coli and is still associated, on an RNA level, with the beneficial properties with respect to supporting RNA stability and translational efficiency. In some aspects, the poly-A sequence contained in an RNA polynucleotide described herein consists essentially of adenosine nucleotides, but is interrupted by a random sequence of the four nucleotides (A, C, G, U). Such a random sequence may be at least, at most, exactly, or between (inclusive or exclusive) any two of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

The poly-A sequence may be located at any position within the 3′ UTR. In some aspects, no nucleotides other than adenosine nucleotides flank a poly-A sequence at its 3′-end, e.g., the poly-A sequence is not masked or followed at its 3′-end by a nucleotide other than adenosine. In some aspects, the poly-A sequence may be located at the 3′ terminus of the 3′ UTR, e.g., the 3′ UTR does not contain more than 12, 11, 10, 9, 8, 7, 6, 5, 4, 3, 2, or 1 nucleotides located 3′ of the poly-A sequence; more preferably the 3′ UTR does not contain further elements located 3′ to the poly-A sequence. In some aspects, poly-A sequence is located at the 3′ terminus of the RNA molecule, e.g., the artificial nucleic acid molecule does not contain more than 12, 11, 10, 9, 8, 7, 6, 5, 4, 3, 2, or 1 nucleotides located 3′ of the poly-A sequence. Alternatively, the poly-A sequence may be located at the 5′ terminus of the 3′ UTR, e.g., immediately 3′ of the ORF of the artificial nucleic acid molecule, or located within the 3′ UTR, e.g., flanked on the 5′ and on the 3′ side by other 3′ UTR elements. In some aspects, the poly-A sequence is flanked on the 3′ side by a poly-C sequence and/or a histone stem-loop sequence. In addition or alternatively, the poly-A sequence can be flanked on the 5′ side by a 3′ UTR element derived from, e.g., a human albumin or globin gene.

In some aspects, the RNA molecule may further include an endonuclease recognition site sequence immediately downstream of the poly-A tail sequence. The RNA molecule may further include a poly-A polymerase recognition sequence (e.g., a polyadenylation signal) (e.g., AAUAAA) near its 3′ end. In some aspects, the polyadenylation signal is located 3′ of the poly-A sequence comprised in the 3′ UTR. In some aspects, the poly-A sequence is separated from the polyadenylation signal by a nucleotide sequence comprising or consisting of at least, at most, exactly, or between (inclusive or exclusive) any two of 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, or 150 nucleotides, wherein the nucleotide sequence does preferably not comprise more than 10, 9, 8, 7, 6, 5, 4, 3, or 2 consecutive adenine nucleotides. In some aspects, the nucleotide sequence that separates the poly-A sequence and the polyadenylation signal comprises from or from about 1 to about 200 nucleotides, e.g., from 10 to 90, from 20 to 85, from 30 to 80, from 40 to 80, from 50 to 75 or from 55 to 85 nucleotides, more preferably from 55 to 80 nucleotides, and the nucleotide sequence does not comprise more than 10, 9, 8, 7, 6, 5, 4, 3, or 2 consecutive adenine nucleotides.

In some aspects, the polyadenylation signal comprises the consensus sequence NN (U/T) ANA, with N=A or U, preferably AA (U/T) AAA or A (U/T) (U/T) AAA. Such a consensus sequence may be recognized by most animal and bacterial cell-systems, for example, by the polyadenylation-factors, such as cleavage/polyadenylation specificity factor (CPSF) cooperating with CstF, PAP, PAB2, CFI and/or CFII. In some aspects, the polyadenylation signal (e.g., the consensus sequence NNUANA) is located less than or less than about 50 nucleotides, e.g., at least, at most, exactly, or between (inclusive or exclusive) any two of 1, 5, 10, 15, 20, 25, 30, 35, 40, 45, or 50 nucleotides, downstream of the 3′-end of the 3′ UTR element as defined herein such that transcription of an RNA molecule will result in a premature-RNA containing the polyadenylation signal downstream of its 3′ UTR and subsequent attachment of a poly-A sequence to the premature-RNA. Accordingly, a resulting RNA may comprise a 3′ UTR, which comprises at least one poly-A sequence, and wherein the 3′ UTR is followed by an additional poly-A sequence.

The poly-A sequence may be of any length. In some aspects, the poly-A tail may be 5 to 300 nucleotides in length. In some aspects, the RNA molecule includes a poly-A tail that comprises, consists essentially of, or consists of a sequence of or of about 25 to about 400 adenosine nucleotides, a sequence of or of about 50 to about 400 adenosine nucleotides, a sequence of or of about 50 to about 300 adenosine nucleotides, a sequence of or of about 50 to about 250 adenosine nucleotides, a sequence of or of about 60 to about 250 adenosine nucleotides, or a sequence of or of about 40 to about 100 adenosine nucleotides. In some aspects, the poly-A tail comprises, consists essentially of, or consists of at least, at most, exactly, or between (inclusive or exclusive) any two of 5, 10, 15, 20, 25, 30, 35, 40, 45, 50, 55, 60, 65, 70, 75, 80, 85, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 adenosine nucleotides. In this context, “consists essentially of” means that most nucleotides in the poly-A sequence, typically at least 75%, at least 80%, at least 85%, at least 90%, at least 95%, at least 96%, at least 97%, at least 98%, or at least 99% by number of nucleotides in the poly-A sequence are adenosine nucleotides, but permits remaining nucleotides to be nucleotides other than adenosine nucleotides, such as uridine, guanosine, and/or cytosine. In this context, “consists of” means that all nucleotides in the poly-A sequence, e.g., 100% by number of nucleotides in the poly-A sequence, are adenosine nucleotides.

In some aspects, the RNA molecule includes a poly-A tail that includes a sequence of greater than 30 adenosine nucleotides. In some aspects, the RNA molecule includes a poly-A tail that includes or includes about 40 adenosine nucleotides. In some aspects, the RNA molecule includes a poly-A tail that includes or includes about 80 adenosine nucleotides. In some aspects, the poly-A tail may have contiguous A residues or interrupted A residues comprising a linker (L). In some aspects, the 3′ poly-A tail has a stretch of at least 10 consecutive adenosine residues and at most 300 consecutive adenosine residues. In some specific aspects, the RNA molecule includes or includes about 40 consecutive adenosine residues. In some aspects, the RNA molecule includes or includes about 80 consecutive adenosine residues. Poly-A tails may play key regulatory roles in enhancing translation efficiency and regulating the efficiency of mRNA quality control and degradation. Short sequences or hyperpolyadenylation may signal for RNA degradation.

In some aspects, a poly-A tail may be located within an RNA molecule or other nucleic acid molecule, such as, e.g., in a vector, for example, in a vector serving as template for the generation of an RNA, e.g., an mRNA, e.g., by transcription of the vector. In some aspects, the RNA molecule may not include a poly-A tail.

In some aspects, a poly-A tail may be located within an RNA molecule or other nucleic acid molecule, such as, e.g., in a vector, for example, in a vector serving as template for the generation of an RNA, e.g. an mRNA, e.g., by transcription of the vector. In some aspects, the RNA molecule may not include a poly-A tail.

In one aspect, a DNA encoding a poly-A tail disclosed herein comprises a sequence having at least, at most, exactly, or between (inclusive or exclusive) any two of 99%, 98%, 97%, 96%, 95%, 90%, 85%, or 80% identity to SEQ ID NO: 21 or 22. In one aspect, the DNA encoding the poly-A tail comprises a sequence of SEQ ID NO: 21 or 22. In one aspect, an RNA disclosed herein comprises a poly-A tail comprising a sequence having at least, at most, exactly, or between (inclusive or exclusive) any two of 99%, 98%, 97%, 96%, 95%, 90%, 85%, or 80% identity to SEQ ID NO: 21 or 22. In one aspects, the poly-A tail comprises a sequence of SEQ ID NO: 21 or 22. In one aspect, the poly-A tail comprises a sequence of SEQ ID NO: 21 or 22+/−2 adenosine (A) nucleotides. In one aspect, the poly-A tail comprises a sequence of SEQ ID NO: 21 or 22+/−1 adenosine (A) nucleotides. In one aspect, the poly-A tail comprises a sequence of SEQ ID NO: 21 or 22. In one aspect, the poly-A tail comprises a sequence of SEQ ID NO: 21 or 22+/−2 adenosine (A) nucleotides. In one aspect, the poly-A tail comprises a sequence of SEQ ID NO: 21 or 22+/−1 adenosine (A) nucleotides. In some aspects, the poly-A tail comprises a sequence of SEQ ID NO: 21 or 22.

In some aspects, 1, 2, 3, 4, 5, or more of the foregoing poly-A sequences may be excluded from the RNA molecules disclosed herein.

(DNA)
SEQ ID NO: 21
AAAAAAAAAAAAAAAAAAAAAAAAAAAAAAAAAAA
AAAAAAAAAAAAAAAAAAAAAAAAAAAAAAAAAAA
AAAAAAAAAA
(RNA)
SEQ ID NO: 22
AAAAAAAAAAAAAAAAAAAAAAAAAAAAAAAAAAA
AAAAAAAAAAAAAAAAAAAAAAAAAAAAAAAAAAA
AAAAAAAAAA
(polyA linker (L); DNA)
SEQ ID NO: 23
GCATATGACT
(polyA linker (L); RNA)
SEQ ID NO: 24
GCAUAUGACU

### G. Other Elements

In some aspects of the present disclosure, the RNA molecules additionally include a chain terminating nucleoside. For example, a chain terminating nucleoside may include those nucleosides deoxygenated at the 2′ and/or 3′ positions of their sugar group. Such species may include 3′ deoxyadenosine (cordycepin), 3′ deoxyuridine, 3′ deoxycytosine, 3′ deoxyguanosine, 3′ deoxythymine, and 2′,3′ dideoxynucleosides, such as 2′,3′ dideoxyadenosine, 2′,3′ dideoxyuridine, 2′,3′ dideoxycytosine, 2′,3′ dideoxyguanosine, and 2′,3′ dideoxythymine. In some aspects, 1, 2, 3, 4, 5, or more of the foregoing chain terminating nucleosides may be excluded from the RNA molecules disclosed herein. In some aspects, incorporation of a chain terminating nucleotide into an mRNA, for example at the 3′-terminus, may result in stabilization of the mRNA, as described, for example, in International Patent Publication No. WO 2013/103659.

In some aspects of the present disclosure, the RNA molecules additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ UTR or a 3′ UTR), a coding region, or a poly-A sequence or tail. In some aspects, a stem loop may affect one or more function(s) of an mRNA, such as initiation of translation, translation efficiency, and/or transcriptional termination. Such histone stem-loop sequences may be histone stem-loop sequences disclosed in WO 2012/019780, the disclosure of which is incorporated herein by reference in its entirety. Other non-limiting examples of histone stem loop structures and nucleic acid sequences encoding such structures can be found in, e.g., WO 2016/091391, the disclosure of which is incorporated by reference herein in its entirety.

In some aspects, the combination of a poly-A sequence or polyadenylation signal and at least one histone stem-loop, even though both represent alternative mechanisms in nature, acts synergistically to increase the protein expression beyond the level observed with either of the individual elements. In some aspects, the synergistic effect of the combination of poly-A and at least one histone stem-loop does not depend on the order of the elements and/or the length of 5 the poly-A sequence.

In some aspects, the RNA does not comprise a histone downstream element (HDE). An HDE includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA.

In some aspects, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. Stability of the stem-loop structure generally depends on the length, number of mismatches or bulges, and/or base composition of the paired region. In some aspects, wobble base pairing (non-Watson-Crick base pairing) may result. In some aspects, the at least one histone stem-loop sequence comprises a length of 15 to 45 nucleotides.

In some aspects, the RNA molecules include (e.g., within the 3′ UTR) a poly(C) sequence. In some aspects, the poly-C sequences has at least, at most, exactly, or between (inclusive or exclusive) any two of 10, 20, 30, 40, 50, 60, 70, 80, 90, 100, 110, 120, 130, 140, 150, 160, 170, 180, 190, or 200 cytidines. In some aspects, the poly-C sequences has or has about 30 cytidines.

In some aspects, the RNA molecules include an internal ribosome entry site (IRES) sequence or IRES-motif. In some aspects, an IRES sequence separates ORFs, e.g., if the RNA encodes two or more peptides or proteins. An IRES-sequence may therefore be useful if the RNA molecule is a bi- or multicistronic nucleic acid molecule.

In some aspects, the RNA does not comprise an intron. In some aspects, the RNA may instead or additionally include a microRNA binding site.

Representative RNA molecules including a combination of the elements disclosed herein can include, without limitation, in 5′-to-3′-direction, the following:
ORF-poly-A sequence;
ORF-IRES-ORF-poly-A sequence;
ORF-3′ UTR-poly-A sequence;
ORF-poly-A sequence-3′ UTR;
ORF-3′ UTR-poly-A sequence-poly(C) sequence-histone stem-loop;
ORF-3′ UTR-poly-A sequence-poly(C) sequence-poly-A sequence;
ORF-3′ UTR-poly-A sequence-histone stem-loop-poly-A sequence;
5′ UTR-ORF-3′ UTR;
5′ UTR-ORF-poly-A sequence;
5′ UTR-ORF-poly-A sequence-poly(C) sequence-histone stem-loop;
5′ UTR-ORF-poly-A sequence-poly(C) sequence-poly-A sequence;
5′ UTR-ORF-poly-A sequence-histone stem-loop-poly-A sequence;
5′ UTR-ORF-3′ UTR-poly-A sequence;
5′ UTR-ORF-3′ UTR-poly-A sequence-poly(C) sequence
5′ UTR-ORF-3′ UTR-poly-A sequence-poly(C) sequence-histone stem-loop;
5′-cap-5′ UTR-ORF-3′ UTR;
5′-cap-5′ UTR-ORF-poly-A sequence;
5′-cap-5′ UTR-ORF-3′ UTR-poly-A sequence;
5′-cap-5′ UTR-ORF-3′ UTR-poly-A sequence-poly(C) sequence; or
5′-cap-5′ UTR-ORF-3′ UTR-poly-A sequence-poly(C) sequence-histone stem-loop.

In some aspects, 1, 2, 3, 4, 5, or more of the foregoing elements may be excluded from the RNA molecules disclosed herein.

### IV. RNA Transcription

In some aspects, the RNA disclosed herein is produced by in vitro transcription or chemical synthesis. In the context of the present disclosure, the term “transcription” relates to a process, wherein the genetic code in a DNA sequence is transcribed into RNA. Subsequently, the RNA may be translated into peptide or protein.

According to the present disclosure, “transcription” comprises “in vitro transcription” or “IVT,” which refers to the process whereby transcription occurs in vitro in a non-cellular system to produce a synthetic RNA product for use in various applications, including, e.g., production of protein or polypeptides. The methodology for in vitro transcription of mRNA is well known in the art. (see, e.g., Losick, R. 1972. In vitro transcription, Ann Rev Biochem, 41 409-46; Kamakaka, R. T. and Kraus, W. L. 2001. In vitro Transcription, Current Protocols in Cell Biology, 2:11.6:11.6.1-11.6.17; Beckert, B. And Masquida, B. 2010. Synthesis of RNA by In vitro Transcription in RNA, Methods in Molecular Biology, 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology 530:101-114; all of which are incorporated herein by reference).

Cloning vectors may be applied for the generation of transcripts. These cloning vectors are generally designated as transcription vectors and are according to the present invention encompassed by the term “vector.” According to specific aspects, the RNA used is in vitro transcribed RNA (IVT-RNA) and may be obtained by in vitro transcription of an appropriate DNA template. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template, and Bowman, J. C., Azizi, B., Lenz, T. K., Ray, P., and Williams, L. D. in RNA in vitro transcription and RNA purification by denaturing PAGE in Recombinant and in vitro RNA syntheses, Methods 941 Conn G. L. (ed), New York, N.Y. Humana Press, 2012, each incorporated herein by reference). The promoter for controlling transcription may be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

Synthetic IVT RNA products may be translated in vitro or introduced directly into cells, where they may be translated. With respect to RNA, the term “expression” or “translation” relates to the process in the ribosomes of a cell by which a strand of mRNA directs the assembly of a sequence of amino acids to make a peptide or protein. Such synthetic RNA products include but are not limited to, e.g., mRNA molecules, saRNA molecules, antisense RNA molecules, shRNA molecules, long non-coding RNA molecules, ribozymes, aptamers, guide RNA molecules (e.g., for CRISPR), ribosomal RNA molecules, small nuclear RNA molecules, small nucleolar RNA molecules, and the like. In some aspects, 1, 2, 3, 4, 5, or more of the foregoing synthetic RNA products may be excluded. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In some aspects, an mRNA is produced by in vitro transcription using a DNA template where DNA refers to a nucleic acid that contains deoxyribonucleotides. In some aspects, an RNA disclosed herein is in vitro transcribed RNA (IVT-RNA) and may be obtained by in vitro transcription of an appropriate DNA template. The promoter for controlling transcription may be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In some aspects, starting material for IVT may include linearized DNA template, nucleotides, Rnase inhibitor, pyrophosphatase, and/or a polymerase (e.g., a T7 RNA polymerase). The nucleotides may be manufactured in house, may be obtained from a supplier, or may be synthesized. The nucleotides may be, but are not limited to, those described herein including natural and unnatural (modified) nucleotides. Any number of RNA polymerases or variants may be used, including, but not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. In some aspects, 1, 2, 3, 4, 5, or more of the foregoing RNA polymerases may be excluded from. Some embodiments exclude the use of Dnase.

In some aspects, the IVT process is conducted in a bioreactor. The bioreactor may comprise a mixer. In some aspects, nucleotides may be added into the bioreactor throughout the IVT process.

In some aspects, one or more post-IVT agents are added into the IVT mixture comprising RNA in the bioreactor after the IVT process. Exemplary post-IVT agents may include DNAse I configured to digest the linearized DNA template and/or proteinase K configured to digest DNAse I and T7 RNA polymerase. In some aspects, the post-IVT agents are incubated with the mixture in the bioreactor after IVT. In some aspects, the bioreactor may contain at least, at most, exactly, or between (inclusive or exclusive) any two of 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 or more liters IVT mixture. The IVT mixture may have an RNA concentration that is or is not at least, at most, exactly, or between (inclusive or exclusive) any two of 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0, 10, 11, 12, 13, 14, 15, 16, 17, 18, 19, 20, 30, 40, 50, 60, 70, 80, 90, and 100 mg/mL or more RNA.

In some aspects, the IVT mixture may include residual spermidine, residual DNA, residual proteins, peptides, HEPES, EDTA, ammonium sulfate, cations (e.g., Mg²⁺, Na⁺, Ca²⁺), RNA fragments, residual nucleotides, free phosphates, or any combinations thereof. In some aspects, 1, 2, 3, 4, 5, or more of the foregoing can be excluded from the IVT mixture.

Isolation and/or purification of the nucleic acids described herein may include, but is not limited to, phenol/chloroform extraction and/or precipitation with either alcohol (ethanol, isopropanol) in the presence of monovalent cations or lithium chloride for nucleic acid clean-up, quality assurance and quality control. Additional, non-limiting examples of purification procedures include AGENCOURT® beads (Beckman Coulter Genomics, Danvers, MA), poly-T beads, LNATM oligo-T capture probes (EXIQON® Inc, Vedbaek, Denmark), HPLC based purification methods such as, but not limited to, strong anion exchange HPLC, weak anion exchange HPLC, reverse phase HPLC (RP-HPLC), and hydrophobic interaction HPLC (HIC-HPLC), size exclusion chromatography, and silica-based affinity chromatography and polyacrylamide gel electrophoresis. Purification can be performed using a variety of commercially available kits including, but not limited to SV Total Isolation System (Promega) and In vitro Transcription Cleanup and Concentration Kit (Norgen Biotek). In some aspects, 1, 2, 3, 4, 5, or more of the foregoing purification may be excluded.

The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment and/or purification method.

In some aspects, at least a portion of the IVT mixture is filtered. The IVT mixture may be filtered via ultrafiltration and/or diafiltration to remove at least some impurities from the IVT mixture and/or to change buffer solution for the at least a portion of IVT mixture to produce a concentrated RNA solution as a retentate.

In some aspects, both “ultrafiltration” and “diafiltration” refer to a membrane filtration process. Ultrafiltration typically uses membranes having pore sizes of at least, at most, exactly, or between (inclusive or exclusive) any two of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1 μm. In some aspects, ultrafiltration membranes are typically classified by molecular weight cutoff (MWCO) rather than pore size. For example, the MWCO may be at least, at most, exactly, or between (inclusive or exclusive) any two of 30 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 500 kDa, 600 kDa, 700 kDa, 800 kDa, 900 kDa, 1000 kDa, 2000 kDa, 3000 kDa, 4000 kDa, 5000 kDa, 6000 kDa, 7000 kDa, 8000 kDa, 9000 kDa, and 10000 kDa. A skilled artisan will understand that filtration membranes may comprise different suitable materials, including, e.g., polymers, cellulose, ceramic, etc., depending upon the application. In some aspects, membrane filtration may be more desirable for large volume purification process.

In some aspects, ultrafiltration and diafiltration of the IVT mixture for purifying RNA may include (1) Direct Flow Filtration (DFF), also known as “dead-end” filtration, that applies a feed stream perpendicular to the membrane face and attempts to pass 100% of the fluid through the membrane, and/or (2) Tangential Flow Filtration (TFF), also known as crossflow filtration, where a feed stream passes parallel to the membrane face as one portion passes through the membrane (permeate) while the remainder (retentate) is retained and/or recirculated back to the feed tank.

In some aspects, the filtering of the IVT mixture is conducted via TFF comprising an ultrafiltration step, a first diafiltration step, and a second diafiltration step. In some aspects, the first diafiltration step is conducted in the presence of ammonium sulfate. The first diafiltration step may be configured to remove a majority of impurities from the IVT mixture. In some aspects, the second diafiltration step is conducted without ammonium sulfate. The second diafiltration step may be configured to transfer the RNA into a DS buffer formulation.

A filtration membrane with an appropriate MWCO may be selected for ultrafiltration in the TFF process. The MWCO of a TFF membrane determines which solutes may pass through the membrane into the filtrate and which are retained in the retentate. The MWCO of a TFF membrane may be selected such that substantially all of the solutes of interest (e.g., desired synthesized RNA species) remain in the retentate, whereas undesired components (e.g., excess ribonucleotides, small nucleic acid fragments such as digested or hydrolyzed DNA template, peptide fragments such as digested proteins and/or other impurities) pass into the filtrate. In some aspects, the retentate comprising desired synthesized RNA species may be re-circulated to a feed reservoir to be re-filtered in additional cycles. In some aspects, a TFF membrane may have a MWCO of at least, at most, exactly, or between (inclusive or exclusive) any two of 30 kDa, 40 kDa, 50 kDa, 60 kDa, 70 kDa, 80 kDa, 90 kDa, or more. In some aspects, a TFF membrane may have a MWCO of at least, at most, exactly, or between (inclusive or exclusive) any two of 100 kDa, 150 kDa, 200 kDa, 250 kDa, 300 kDa, 350 kDa, 400 kDa, or more. In some aspects, a TFF membrane may have a MWCO of or of about 250-350 kDa. In some aspects, a TFF membrane (e.g., a cellulose-based membrane) may have a MWCO of or of about 30-300 kDa; 50-300 kDa, 100-300 kDa, or 200-300 kDa.

Diafiltration may be performed either discontinuously, or alternatively, continuously. For example, in continuous diafiltration, a diafiltration solution may be added to a sample feed reservoir at the same rate as filtrate is generated. In this way, the volume in the sample reservoir remains constant but small molecules (e.g., salts, solvents, etc.) that may freely permeate through a membrane are removed. Using solvent removal as an example, each additional diafiltration volume (DV) reduces the solvent concentration further. In discontinuous diafiltration, a solution is first diluted and then concentrated back to the starting volume. This process is then repeated until the desired concentration of small molecules (e.g., salts, solvents, etc.) remaining in the reservoir is reached. Each additional diafiltration volume (DV) reduces the small molecule (e.g., solvent) concentration further. Continuous diafiltration typically requires a minimum volume for a given reduction of molecules to be filtered. Discontinuous diafiltration, on the other hand, permits fast changes of the retentate condition, such as pH, salt content, and the like. In some aspects, the first diafiltration step is conducted with at least, at most, exactly, or between (inclusive or exclusive) any two of 2, 3, 4, 5, 6, 7, 8, 9, 10, or more diavolumes. In some aspects, the second diafiltration step is conducted with at least, at most, exactly, or between (inclusive or exclusive) any two of 5, 6, 7, 8, 9, 10, 11, 12, 13, 14, 15, 16, 17, 18, 19, 20, or more diavolumes. In some aspects, the first diafiltration step is conducted with 5 diavolumes, and second diafiltration step is conducted with 10 diavolumes.

In some aspects, for ultrafiltration and/or diafiltration, the IVT mixture is filtered at a rate of at least, at most, exactly, or between (inclusive or exclusive) any two of 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 600, 700, 800, 900, or 1000 L/m2 of filter area per hour, or more. The concentrated RNA solution may comprise at least, at most, exactly, or between (inclusive or exclusive) any two of 2.0, 2.1, 2.2, 2.3, 2.4, or 2.5 mg/mL single stranded RNA.

The bioburden of the concentrated RNA solution via filtration to obtain an RNA product solution may also be reduced, in some aspects. The filtration for reducing bioburden may be conducted using one or more filters. The one or more filters may include a filter with a pore size that is or is not at least, at most, exactly, or between (inclusive or exclusive) any two of 0.2 μm, 0.45 μm, 0.65 μm, 0.8 μm, or any other pore size configured to remove bioburdens.

As one example, reducing the bioburden may include draining a retentate tank containing retentate obtained from the ultrafiltration and/or diafiltration to obtain the retentate. Reducing the bioburden may include flushing a filtration system for ultrafiltration and/or diafiltration using a wash buffer solution to obtain a wash pool solution comprising residue RNA remaining in the filtration system. The retentate may be filtered to obtain a filtered retentate. The wash pool solution may be filtered using a first 0.2 μm filter to obtain a filtered wash pool solution. The retentate may be filtered using the first 0.2 μm filter or another 0.2 μm filter.

The filtered wash pool solution and the filtered retentate may be combined to form a combined pool solution. The combined pool solution may be filtered using a second 0.2 μm filter to obtain a filtered combined pool solution, which is further filtered using a third 0.2 μm filter to produce an RNA product solution.

A quality assurance and/or quality control check may be conducted using methods such as, but not limited to, gel electrophoresis, UV absorbance, and/or analytical HPLC.

In some aspects, the nucleic acids may be sequenced by methods including, but not limited to reverse-transcriptase-PCR.

In some aspects, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size and/or to assess degradation. Degradation of the nucleic acid may be assessed by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE). In some aspects, 1, 2, 3, 4, 5, or more of the foregoing assessment methods may be excluded.

### V. RNA Encapsulation

The RNA in an RNA product solution may be encapsulated, and the RNA solution may further comprise at least one encapsulating agent. In one aspect, the encapsulating agent comprises a lipid, a lipid nanoparticle (LNP), lipoplexes, polymeric particles, polyplexes, monolithic delivery systems, or a combination thereof. In some aspects, 1, 2, 3, 4, 5, or more of the foregoing elements may be excluded as an encapsulating agent.

In one aspect, the encapsulating agent is a lipid, and produced is lipid nanoparticle (LNP)-encapsulated RNA. Without intending to be bound by any theory, it is believed that the cationic or cationically ionizable lipid or lipid-like material and/or the cationic polymer combine together with the nucleic acid to form aggregates, and this aggregation results in colloidally stable particles.

A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A lipid is a substance that is insoluble in water and extractable with an organic solvent. Compounds other than those specifically described herein are understood by one of skill in the art as lipids and are encompassed by the compositions and methods of the present disclosure. A lipid component and a non-lipid may be attached to one another, either covalently or non-covalently.

In some aspects, LNPs may be designed to protect RNA molecules (e.g., saRNA, mRNA) from extracellular Rnases and/or may be engineered for systemic delivery of the RNA to target cells. In some aspects, such LNPs may be particularly useful to deliver RNA molecules (e.g., saRNA, mRNA) when RNA molecules are intravenously administered to a subject in need thereof. In some aspects, such LNPs may be particularly useful to deliver RNA molecules (e.g., saRNA, mRNA) when RNA molecules are intramuscularly administered to a subject in need thereof. In some aspects, such LNPs may be particularly useful to deliver RNA molecules (e.g., saRNA, mRNA) when RNA molecules are intradermally administered to a subject in need thereof. In some aspects, such LNPs may be particularly useful to deliver RNA molecules (e.g., saRNA, mRNA) when RNA molecules are intranasally administered to a subject in need thereof. In one aspect, the RNA in the RNA product solution is at a concentration of <1 mg/mL.

In another aspect, the RNA is at a concentration of at least or at least about 0.05 mg/mL. In another aspect, the RNA is at a concentration of at least or at least about 0.5 mg/mL. In another aspect, the RNA is at a concentration of at least or at least about 1 mg/mL. In another aspect, the RNA concentration is from or from about 0.05 mg/mL to about 0.5 mg/mL. In another aspect, the RNA is at a concentration of at least 10 mg/mL. In another aspect, the RNA is at a concentration of at least 50 mg/mL. In some aspects, the RNA is or is not at a concentration of at least, at most, exactly, between (inclusive or exclusive) any two of, or about 0.05 mg/mL, 0.5 mg/mL, 1 mg/mL, 10 mg/mL, 50 mg/mL, 75 mg/mL, 100 mg/mL, 150 mg/mL, 200 mg/mL, 250 mg/mL, 300 mg/mL, 400 mg/mL, or more.

The present disclosure provides for an RNA product solution and a lipid preparation mixture or compositions thereof comprising at least one RNA encoding, e.g., an antigen (e.g., an RSV prefusion F protein) complexed with, encapsulated in, and/or formulated with one or more lipids, and forming lipid nanoparticles (LNPs), liposomes, lipoplexes and/or nanoliposomes. In some aspects, the composition comprises a lipid nanoparticle.

A lipid nanoparticle or LNP refers to particles of any morphology generated when a cationic lipid and optionally one or more further lipids are combined, e.g., in an aqueous environment and/or in the presence of RNA. In some aspects, lipid nanoparticles are included in a formulation that may be used to deliver an active agent or therapeutic agent, such as a nucleic acid (e.g., mRNA) to a target site of interest (e.g., cell, tissue, organ, tumor, and the like). In some aspects, the lipid nanoparticles of the present disclosure comprise a nucleic acid (e.g., mRNA). Such lipid nanoparticles typically comprise a cationic lipid and one or more excipients, e.g., one or more neutral lipids, charged lipids, steroids, polymer conjugated lipids, or combinations thereof. In some aspects, the LNPs comprise at least one cationic (e.g., ionizable) lipid, at least one neutral (e.g., non-cationic) lipid, at least one structural lipid (e.g., a steroid), and/or at least one polymer conjugated lipid (e.g., a polyethylene glycol (PEG)-modified lipid). In some aspects, 1, 2, 3, or more of the foregoing excipients may be excluded from the LNPs.

In some aspects, the LNPs comprise 20-60 mol % cationic (e.g., ionizable) lipid(s). For example, the LNPs may comprise 20-50 mol %, 20-40 mol %, 20-30 mol %, 30-60 mol %, 30-50 mol %, 30-40 mol %, 40-60 mol %, 40-50 mol %, or 50-60 mol % cationic (e.g., ionizable) lipid(s). In some aspects, the LNPs comprise or do not comprise at least, at most, exactly, or between (inclusive or exclusive) any two of 20 mol %, 30 mol %, 40 mol %, 50, or 60 mol % cationic (e.g., ionizable) lipid(s). In some aspects, the LNPs comprise 45 to 55 mole percent (mol %) cationic (e.g., ionizable) lipid(s). For example, LNPs may comprise or not comprise at least, at most, exactly, or between (inclusive or exclusive) any two of 45, 46, 47, 48, 49, 50, 51, 52, 53, 54, or 55 mol % cationic (e.g., ionizable) lipid(s).

In some aspects, the LNPs comprise 5-25 mol % neutral (e.g., non-cationic) lipid(s). For example, the LNPs may comprise 5-20 mol %, 5-15 mol %, 5-10 mol %, 10-25 mol %, 10-20 mol %, 10-25 mol %, 15-25 mol %, 15-20 mol %, or 20-25 mol % neutral (e.g., non-cationic) lipid(s). In some aspects, the LNPs are or are not at least, at most, exactly, or between (inclusive or exclusive) any two of 5 mol %, 10 mol %, 15 mol %, 20 mol %, or 25 mol % neutral (e.g., non-cationic) lipid(s). In some aspects, the LNPs comprise 5 to 15 mol % neutral (e.g., non-cationic) lipid(s). For example, LNPs may comprise at least, at most, exactly, or between (inclusive or exclusive) any two of 5, 6, 7, 8, 9, 10, 11, 12, 13, 14, or 15 mol % neutral (e.g., non-cationic) lipid(s).

In some aspects, the LNPs comprise 25-55 mol % structural lipid(s) (e.g., a steroid). For example, the LNPs may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % structural lipid(s) (e.g., a steroid). In some aspects, the LNPs are or are not at least, at most, exactly, or between (inclusive or exclusive) any two of 25 mol %, 30 mol %, 35 mol %, 40 mol %, 45 mol %, 50 mol %, or 55 mol % structural lipid(s) (e.g., a steroid). In some aspects, the LNPs comprise 35 to 40 mol % structural lipid(s) (e.g., a steroid). For example, LNPs may comprise at least, at most, exactly, or between (inclusive or exclusive) any two of 35, 36, 37, 38, 39, or 40 mol % structural lipid(s) (e.g., a steroid).

In some aspects, the LNPs comprise 0.5-15 mol % polymer conjugated lipid(s) (e.g., a polyethylene glycol (PEG)-modified lipid). For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol % polymer conjugated lipid(s) (e.g., a polyethylene glycol (PEG)-modified lipid). In some aspects, the lipid LNPs are or are not at least, at most, exactly, or between (inclusive or exclusive) any two of 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % polymer conjugated lipid(s) (e.g., a polyethylene glycol (PEG)-modified lipid). In some aspects, the LNPs comprise 1 to 2 mol % polymer conjugated lipid(s) (e.g., a polyethylene glycol (PEG)-modified lipid). For example, LNPs may comprise at least, at most, exactly, or between (inclusive or exclusive) any two of 1, 1.5, or 2 mol % polymer conjugated lipid(s) (e.g., a polyethylene glycol (PEG)-modified lipid).

In some aspects, the LNPs comprise 20-75 mol % cationic (e.g., ionizable) lipid(s) (e.g., at least, at most, exactly, or between (inclusive or exclusive) any two of 20%, 25%, 30%, 35%, 40%, 45%, 50%, 55%, 60%, 65%, 70%, and 75%), 0.5-25 mol % neutral (e.g., non-cationic) lipid(s) (e.g., at least, at most, exactly, or between (inclusive or exclusive) of 0.5%, 2.25%, 4%, 5.75%, 7.5%, 9.25%, 11%, 12.75%, 14.5%, 16.25%, 18%, 19.75%, 21.5%, 23.25%, and 25%), 5-55 mol % structural lipid(s) (e.g., a sterol) e.g., non-cationic) lipid(s) (e.g., at least, at most, exactly, or between (inclusive or exclusive) of 5%, 10%, 15%, 20%, 25%, 30%, 35%, 40%, 45%, 50%, and 55%), and 0.5-20 mol % polymer conjugated lipid(s) (e.g., a polyethylene glycol (PEG)-modified lipid) (e.g., at least, at most, exactly, or between (inclusive or exclusive) of 0.5%, 2%, 3.5%, 5%, 6.5%, 8%, 9.5%, 11%, 12.5%, 14%, 15.5%, 17%, 18.5%, and 20%). In some aspects, 1, 2, 3, or more of the lipids may be excluded from the LNPs.

In some non-limiting aspects, the molar lipid ratio is 50/10/38.5/1.5 (mol % cationic lipid/neutral lipid/structural lipid/polymer conjugated lipid), 60/7.5/31/1.5 (mol % cationic lipid/neutral lipid/structural lipid/polymer conjugated lipid), 57.5/7.5/31.5/3.5 (mol % cationic lipid/neutral lipid/structural lipid/polymer conjugated lipid), 57.2/7.1/34.3/1.4 (mol % cationic lipid/neutral lipid/structural lipid/polymer conjugated lipid), 40/15/40/5 (mol % cationic lipid/neutral lipid/structural lipid/polymer conjugated lipid), 50/10/35/4.5/0.5 (mol % cationic lipid/neutral lipid/structural lipid/polymer conjugated lipid), 50/10/35/5 cationic lipid/neutral lipid/structural lipid/polymer conjugated lipid), 47.5/10/40.7/1.8 (mol % cationic lipid/neutral lipid/structural lipid/polymer conjugated lipid), 40/10/40/10 lipid/neutral lipid/structural lipid/polymer conjugated lipid), 35/15/40/10 (mol % cationic lipid/neutral lipid/structural lipid/polymer conjugated lipid), or 52/13/30/5 (mol % cationic lipid/neutral lipid/structural lipid/polymer conjugated lipid).

In some aspects, the active agent or therapeutic agent, such as a nucleic acid (e.g., mRNA), may be encapsulated in the lipid portion of the lipid nanoparticle and/or an aqueous space enveloped by some or all of the lipid portion of the lipid nanoparticle, thereby protecting it from enzymatic degradation or other undesirable effects induced by the mechanisms of the host organism or cells, e.g., an adverse immune response. The nucleic acid (e.g., mRNA) or a portion thereof may also be associated and complexed with the lipid nanoparticle. A lipid nanoparticle may comprise any lipid capable of forming a particle to which the nucleic acids are attached, and/or in which the one or more nucleic acids are encapsulated.

In some aspects, provided RNA molecules (e.g., mRNA) may be formulated with LNPs. In some aspects, the lipid nanoparticles may or may not have a mean diameter of or of about 1 to 500 nm (e.g., at least, at most, exactly, or between (inclusive or exclusive) of 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nm). In some aspects, the lipid nanoparticles have a mean diameter of or of from about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, about 70 nm to about 80 nm, or at least, at most, exactly, or between (inclusive or exclusive) of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321). Here, “mean diameter,” “diameter,” or “size” for particles is used synonymously with the value of the Z-average.

LNPs described herein may exhibit a polydispersity index less than or less than about 0.5, 0.4, 0.3, or 0.2 or less. By way of example, the LNPs may or may not exhibit a polydispersity index of at least, at most, exactly, or between (inclusive or exclusive) of 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5. The polydispersity index is, in some aspects, calculated based on dynamic light scattering measurements by the so-called cumulant analysis referred to in the definition of “average diameter.” Under certain prerequisites, it may be taken as a measure of the size distribution of an ensemble of nanoparticles.

In some aspects, an LNP of the disclosure comprises or does not comprise an N:P ratio of or of from about 2:1 to about 30:1, e.g., at least, at most, exactly, or between (inclusive or exclusive) of 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1. In some aspects, an LNP of the disclosure comprises an N:P ratio of or of about 6:1. In some aspects, an LNP of the disclosure comprises an N:P ratio of or of about 3:1. As used herein, the “N:P ratio” is the molar ratio of ionizable (in the physiological pH range) nitrogen atoms in a lipid to phosphate groups in an RNA, e.g., in a LNP including a lipid component and an RNA.

In some aspects, an LNP of the disclosure comprises or does not comprise a wt/wt ratio of the cationic lipid component to the RNA of or of from about 5:1 to about 100:1, e.g., at least, at most, exactly, or between (inclusive or exclusive) of 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, or 100:1. In some aspects, an LNP of the disclosure comprises a wt/wt ratio of the ionizable cationic lipid component to the RNA of or of about 20:1. In some aspects, an LNP of the disclosure comprises a wt/wt ratio of the ionizable cationic lipid component to the RNA of or of about 10:1.

In certain aspects, nucleic acids (e.g., RNA molecules), when present in provided LNPs, are resistant in aqueous solution to degradation with a nuclease. In some aspects, LNPs are liver-targeting lipid nanoparticles. In some aspects, LNPs are cationic lipid nanoparticles comprising one or more cationic lipids (e.g., those described herein). In some aspects, cationic LNPs may comprise at least one cationic lipid, at least one polymer conjugated lipid, and at least one helper lipid (e.g., at least one neutral lipid).

In certain aspects, the RNA solution and lipid preparation mixture or compositions thereof may have at least, at most, exactly, between (inclusive or exclusive) of, or about 1%, 2%, 3%, 4% 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of a particular lipid, lipid type, or non-lipid component such as lipid-like materials and/or cationic polymers and/or an adjuvant, antigen, peptide, polypeptide, sugar, nucleic acid or other material disclosed herein or as would be known to one of skill in the art.

LNPs described herein can be generated using components, compositions, and methods as are generally known in the art, see, e.g., PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/052117; PCT/US2012/069610; PCT/US2017/027492; PCT/US2016/059575 and PCT/US2016/069491, all of which are incorporated by reference herein in their entirety. Other non-limiting examples of methods for preparing LNPs can be found in, e.g., WO 2022/032154, the disclosure of which is incorporated by reference herein in its entirety.

For example, methods of preparing LNPs may involve obtaining a colloid from at least one cationic or cationically ionizable lipid or lipid-like material and/or at least one cationic polymer and mixing the colloid with nucleic acid to obtain nucleic acid particles. The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” refers only to the particles in the mixture and not the entire suspension.

For the preparation of colloids comprising at least one cationic or cationically ionizable lipid or lipid-like material and/or at least one cationic polymer, methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are first dissolved in a suitable organic solvent and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that subsequently turns into a liposomal suspension.

The term “ethanol injection technique” refers to a process in which an ethanol solution comprising lipids is rapidly injected into an aqueous solution through a needle. This action disperses the lipids throughout the solution and promotes lipid structure formation, for example, lipid vesicle formation such as liposome formation. Generally, the RNA lipoplex particles described herein are obtainable by adding RNA to a colloidal liposome dispersion. Using the ethanol injection technique, such colloidal liposome dispersion is, in some aspects, formed as follows: an ethanol solution comprising lipids, such as cationic lipids and additional lipids, is injected into an aqueous solution under stirring. In some aspects, the RNA lipoplex particles described herein are obtainable without a step of extrusion. The term “extruding” or “extrusion” refers to the creation of particles having a fixed, cross-sectional profile. In particular, it refers to the downsizing of a particle, whereby the particle is forced through filters with defined pores.

Other methods for preparing a colloid having organic solvent free characteristics may also be used according to the present disclosure.

In some aspects, LNP-encapsulated RNA may be produced by rapid mixing of an RNA solution described herein (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some aspects, suitable buffering agents comprise tris, histidine, citrate, acetate, phosphate, and/or succinate. In some aspects, 1, 2, 3, or more of the foregoing buffering agents are excluded. The pH of a liquid formulation relates to the pKa of the encapsulating agent (e.g., cationic lipid). The pH of the acidifying buffer may be at least half a pH scale less than the pKa of the encapsulating agent (e.g., cationic lipid), and the pH of the final buffer may be at least half a pH scale greater than the pKa of the encapsulating agent (e.g., cationic lipid). In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, may result in much higher encapsulation efficiency than is achieved in the absence of interactions between nucleic acids and at least one of the lipid components. In certain aspects, nucleic acids, when present in the lipid nanoparticles, are resistant in aqueous solution to degradation with a nuclease.

Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

Some aspects described herein relate to compositions, methods and uses involving more than one, e.g., 2, 3, 4, 5, 6 or even more nucleic acid species, such as RNA species. In an LNP formulation, it is possible that each nucleic acid species is separately formulated as an individual LNP formulation. In that case, each individual LNP formulation will comprise one nucleic acid species. The individual LNP formulations may be present as separate entities, e.g., in separate containers. Such formulations are obtainable by providing each nucleic acid species separately (typically each in the form of a nucleic acid-containing solution) together with suitable cationic or cationically ionizable lipids or lipid-like materials and cationic polymers that allow the formation of LNPs. Respective particles will contain exclusively the specific nucleic acid species that is being provided when the particles are formed (individual particulate formulations).

In some aspects, a composition such as a pharmaceutical composition comprises more than one individual LNP formulation. Respective pharmaceutical compositions are referred to as mixed LNP formulations. Mixed LNP formulations according to the invention are obtainable by forming, separately, individual LNP formulations, as described above, followed by a step of mixing of the individual LNP formulations. By the step of mixing, a formulation comprising a mixed population of nucleic acid-containing LNPs is obtainable. Individual LNP populations may be together in one container, comprising a mixed population of individual LNP formulations.

Alternatively, it is possible that different nucleic acid species are formulated together as a combined LNP formulation. Such formulations are obtainable by providing a combined formulation (typically combined solution) of different RNA species together with suitable cationic or cationically ionizable lipids or lipid-like materials and cationic polymers that allow the formation of LNPs. As opposed to a mixed LNP formulation, a combined LNP formulation will typically comprise LNPs that comprise more than one RNA species. In a combined LNP composition, different RNA species are typically present together in a single particle.

### A. Cationic Polymeric Materials

Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some aspects herein. In addition, some investigators have synthesized polymeric materials specifically for nucleic acid delivery. Poly(P-amino esters), in particular, have gained widespread use in nucleic acid delivery owing to their ease of synthesis and biodegradability. In some aspects, such synthetic materials may be suitable for use as cationic materials herein.

A “polymeric material,” as used herein, is given its ordinary meaning, e.g., a molecular structure comprising one or more repeat units (monomers), connected by covalent bonds. In some aspects, such repeat units may all be identical; alternatively, in some cases, there may be more than one type of repeat unit present within the polymeric material. In some cases, a polymeric material is biologically derived, e.g., a biopolymer such as a protein. In some cases, additional moieties may also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some aspects, a polymer (or polymeric moiety) utilized in accordance with the present disclosure may be a copolymer. Repeat units forming the copolymer may be arranged in any fashion. For example, in some aspects, repeat units may be arranged in a random order; alternatively or additionally, in some aspects, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

In certain aspects, a polymeric material for use in accordance with the present disclosure is biocompatible. Biocompatible materials are those that typically do not result in significant cell death at moderate concentrations. In certain aspects, a biocompatible material is biodegradable, e.g., is able to degrade, chemically and/or biologically, within a physiological environment, such as within the body. In certain aspects, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some aspects, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some aspects, a polyalkyleneimine comprises polyethylenimine and/or polypropylenimine. In some aspects, the polyalkyleneimine is polyethyleneimine (PEI). In some aspects, the polyalkyleneimine is a linear polyalkyleneimine, e.g., linear polyethyleneimine (PEI).

Cationic materials (e.g., polymeric materials, including polycationic polymers) contemplated for use herein include those which are able to electrostatically bind nucleic acid. In some aspects, cationic polymeric materials contemplated for use herein include any cationic polymeric materials with which nucleic acid may be associated, e.g. by forming complexes with the nucleic acid or forming vesicles in which the nucleic acid is enclosed or encapsulated. In some aspects, particles described herein may comprise polymers other than cationic polymers, e.g., non-cationic polymeric materials and/or anionic polymeric materials. Collectively, anionic and neutral polymeric materials are referred to herein as non-cationic polymeric materials.

### B. Lipids & Lipid-Like Materials

The terms “lipid” and “lipid-like material” are used herein to refer to molecules which comprise one or more hydrophobic moieties or groups and optionally also one or more hydrophilic moieties or groups. According to the disclosure, lipids and lipid-like materials may be cationic, anionic or neutral. Neutral lipids or lipid-like materials exist in an uncharged or neutral zwitterionic form at a selected pH.

The term “lipid” refers to a group of organic compounds that are characterized by being insoluble in water but soluble in many organic solvents. Generally, lipids may be divided into eight categories: fatty acids and their derivatives (including tri-, di-, monoglycerides, and phospholipids), glycerolipids, glycerophospholipids, sphingolipids, saccharolipids, polyketides, sterol lipids as well as sterol-containing metabolites such as cholesterol, and prenol lipids. Examples of fatty acids include, but are not limited to, fatty esters and fatty amides. Examples of glycerolipids include, but are not limited to, glycosylglycerols and glycerophospholipids (e.g., phosphatidylcholine, phosphatidylethanolamine, phosphatidylserine). Examples of sphingolipids include, but are not limited to, ceramides phosphosphingolipids (e.g., sphingomyelins, phosphocholine), and glycosphingolipids (e.g., cerebrosides, gangliosides). Examples of sterol lipids include, but are not limited to, cholesterol and its derivatives and tocopherol and its derivatives. In some aspects, 1, 2, 3, 4, 5, or more of the lipids may be excluded from the LNPs of the present disclosure.

The term “lipid-like material,” “lipid-like compound,” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

In some aspects, the RNA solution and lipid preparation mixture or compositions thereof may comprise cationic lipids, neutral lipids, cholesterol, and/or polymer (e.g., polyethylene glycol) conjugated lipids which form lipid nanoparticles that encompass the RNA molecules. Therefore, in some aspects, the LNP may comprise a cationic lipid and one or more excipients, e.g., one or more neutral lipids, charged lipids, steroids or steroid analogs (e.g., cholesterol), polymer conjugated lipids (e.g. PEG-lipid), or combinations thereof. In some aspects, 1, 2, 3, or more of the foregoing excipients may be excluded from the LNPs of the present disclosure. In some aspects, the lipids are present in a composition in an amount that is effective to form a lipid nanoparticle and deliver a therapeutic agent, e.g., an RNA molecule, for treating a particular disease or condition of interest, e.g., those related to RSV. In some aspects, the LNPs encompass, or encapsulate, the nucleic acid molecules.

i. Cationic Lipids

Cationic or cationically ionizable lipids or lipid-like materials refer to a lipid or lipid-like material capable of being positively charged and able to electrostatically bind nucleic acid. As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl, or more acyl chains, and the head group of the lipid typically carries the positive charge. Exemplary cationic lipids include one or more amine group(s) which bear the positive charge. Cationic lipids may encapsulate negatively charged RNA.

In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids or lipid-like materials are comprised by the term “cationic lipid” or “cationic lipid-like material” unless contradicted by the circumstances.

In some aspects, a cationic lipid may comprise from or from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle. In some aspects, a cationic lipid may or may not be at least, at most, exactly, or between (inclusive or exclusive) of 10 mol %, 20 mol %, 30 mol %, 40 mol %, 50 mol %, 60 mol %, 70 mol %, 80 mol %, 90 mol %, or 100 mol %, or any range or value derivable therein, of the total lipid present in the particle. In a preferred aspect, the cationic lipid comprises 47.5 mol % of the total lipid present in the LNP.

Examples of cationic lipids include, but are not limited to: ((4-hydroxybutyl) azanediyl)bis(hexane-6,1-diyl)bis(2-hexyldecanoate), 1,2-dioleoyl-3-trimethylammonium propane (DOTAP), N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl) cholesterol (DC-Chol), dimethyldioctadecylammonium (DDAB), 1,2-dioleoyl-3-dimethylammonium-propane (DODAP), 1,2-diacyloxy-3-dimethylammonium propanes, 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleoyloxypropyl-3-dimethyl-hydroxyethyl ammonium bromide (DORIE), 2,3-dioleoyloxy-N-[2 (spermine carboxamide)ethyl]-N,N-dimethyl-1-propanamium trifluoroacetate (DOSPA), 1,2-dilinoleyloxy-N, N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-I-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DMA), N-(2-Hydroxyethyl)-N,N-dimethyl-2,3-bis(tetradecyloxy)-1-propanaminium bromide (DMRIE), (+)-N-(3-aminopropyl)-N,N-dimethyl-2,3-bis(cis-9-tetradecenyloxy)-1-propanaminium bromide (GAP-DMORIE), (+)-N-(3-aminopropyl)-N,N-dimethyl-2,3-bis(dodecyloxy)-1-propanaminium bromide (GAP-DLRIE), (+)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy) propan-1-aminium (DOBAQ), 2-({8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy) propan-1-aminium bromide (DMORIE), di((Z)-non-2-en-I-yl) 8,8′-((((2(dimethylamino)ethyl)thio) carbonyl) azanediyl)dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-I-yl)-9-((4-(dimethylaminobutanoyl)oxy) heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid 02-200); C 12-200; or heptadecan-9-yl 8-((2-hydroxyethyl) (6-oxo-6-(undecyloxy) hexyl)amino) octanoate (SM-102). In some aspects, 1, 2, 3, 4, 5, or more of the foregoing cationic lipids may be excluded from the LNPs of the present disclosure.

In some aspects, an ionizable cationic lipid of the disclosure comprises a compound of Formula (I):

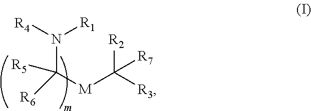

or a pharmaceutically acceptable salt, tautomer, prodrug, or stereoisomer thereof, wherein:
R₁ is a C₅₋₃₀ alkyl, C₅₋₂₀ alkenyl, —R*YR″, —YR″, or —R″M′R′;
R₂ and R₃ are independently a H, C₁₋₁₄ alkyl, C₂₋₁₄ alkenyl, —R*YR″, —YR″, or —R*OR″, and/or
R₂ and R₃, together with the atom to which they are attached, form a heterocycle or carbocycle;
R₄ is a C₃₋₆ carbocycle, —(CH₂)ₙQ, —(CH₂)ₙCHQR, —CHQR, —CQ(R)₂, or unsubstituted C₁₋₆ alkyl, where Q is a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR) S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R) OR, or —C(R)N(R)₂C(O)OR, and/or each n is independently a 1, 2, 3, 4, or 5;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′) C(O)—, —C(O)—, —C(S)—, —C(S)S—, —SC(S)—, —CH (OH)—, —P(O)(OR′)O—, —S(O)₂—, —S—S—, an aryl group, or a heteroaryl group;
R₇ is a C₁₋₃ alkyl, C₂₋₃ alkenyl, or H;
R₈ is a C₃₋₆ carbocycle or heterocycle;
R₉ is a H, CN, NO₂, C₁₋₆ alkyl, —OR, —S(O)₂R, —S(O)₂N(R)₂, C₂₋₆ alkenyl, C₃₋₆ carbocycle, or heterocycle;
each R is a C₁₋₃ alkyl, C₂₋₃ alkenyl, or H;
each R′ is a C₁₋₁₈ alkyl, C₂₋₁₈ alkenyl, —R*YR″, —YR″, or H;
each R″ is a C₃₋₁₄ alkyl or C₃₋₁₄ alkenyl;
each R* is independently a C₁₋₁₂ alkyl or C₂₋₁₂ alkenyl;
each Y is independently a C₃₋₆ carbocycle;
each X is independently a F, Cl, Br, or I; and
m is a 5, 6, 7, 8, 9, 10, 11, 12, or 13.

In some aspects, a subset of compounds of Formula (I) includes those in which when R4 is —(CH₂)ₙQ, —(CH₂)ₙCHQR, —CHQR, or —CQ(R)₂, then (i) Q is not —N(R)₂ when n is 1, 2, 3, 4, or 5, or (ii) Q is not 5, 6, or 7-membered heterocycloalkyl when n is 1 or 2.

In some aspects, another subset of compounds of Formula (I) includes those in which R1 is a C₅₋₃₀ alkyl, C₅₋₂₀ alkenyl, —R*YR″, —YR″, or —R″M′R′;
R₂ and R₃ are independently an H, C₁₋₁₄ alkyl, C₂₋₁₄ alkenyl, —R*YR″, —YR″, or —R*OR″, and/or R₂ and R₃, together with the atom to which they are attached, form a heterocycle or carbocycle;
R₄ is a C₃₋₆ carbocycle, —(CH₂)ₙQ, —(CH₂)ₙCHQR, —CHQR, —CQ(R)₂, or unsubstituted C₁₋₆ alkyl, where Q is a C₃₋₆ carbocycle, a 5- to 14-membered heteroaryl having one or more heteroatoms comprising N, O, or S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR) S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R) OR, or a 5- to 14-membered heterocycloalkyl having one or more heteroatoms comprising N, O, and S which is substituted with one or more substituents comprising oxo (═O), OH, amino, mono- or di-alkylamino, or C₁₋₃ alkyl, and/or each n is independently 1, 2, 3, 4, or 5;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′) C(O)—, —C(O)—, —C(S)—, —C(S)S—, —SC(S)—, —CH (OH)—, —P(O)(OR′)O—, —S(O)₂—, —S—S—, an aryl group, or a heteroaryl group;
R₇ is a C₁₋₃ alkyl, C₂₋₃ alkenyl, or H;
R₈ is a C₃₋₆ carbocycle or heterocycle;
R₉ is a H, CN, NO₂, C₁₋₆ alkyl, —OR, —S(O)₂R, —S(O)₂N(R)₂, C₂₋₆ alkenyl, C₃₋₆ carbocycle or heterocycle;
each R is independently a C₁₋₃ alkyl, C₂₋₃ alkenyl, or H;
each R′ is independently a C₁₋₁₈ alkyl, C₂₋₁₈ alkenyl, —R* YR″, —YR″, or H;
each R″ is independently a C₃₋₁₄ alkyl or C₃₋₁₄ alkenyl;
each R* is independently a C₁₋₁₂ alkyl or C₂₋₁₂ alkenyl;
each Y is independently a C₃₋₆ carbocycle;
each X is independently a F, Cl, Br, or I; and
m is 5, 6, 7, 8, 9, 10, 11, 12, or 13, and/or pharmaceutically acceptable salts, tautomers, prodrugs, or stereoisomers thereof.

In some aspects, another subset of compounds of Formula (I) includes those in which:
R₁ is a C₅₋₃₀ alkyl, C₅₋₂₀ alkenyl, —R*YR″, —YR″, or —R″M′R′;
R₂ and R₃ are independently an H, C₁₋₁₄ alkyl, C₂₋₁₄ alkenyl, —R*YR″, —YR″, or —R*OR″, and/or R₂ and R₃, together with the atom to which they are attached, form a heterocycle or carbocycle;
R₄ is a C₃₋₆ carbocycle, —(CH₂)ₙQ, —(CH₂)ₙCHQR, —CHQR, —CQ(R)₂, or unsubstituted C₁₋₆ alkyl, where Q is a C₃₋₆ carbocycle, a 5- to 14-membered heterocycle having one or more heteroatoms comprising N, O, or S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR) S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R) OR, or —C(═NR₉)N(R)₂, and/or each n is independently 1, 2, 3, 4, or 5; and/or when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′) C(O)—, —C(O)—, —C(S)—, —C(S)S—, —SC(S)—, —CH (OH)—, —P(O)(OR′)O—, —S(O)₂—, —S—S—, an aryl group, or a heteroaryl group;
R₇ is a C₁₋₃ alkyl, C₂₋₃ alkenyl, or H;
R₈ is C₃₋₆ carbocycle or heterocycle;
R₉ is H, CN, NO₂, C₁₋₆ alkyl, —OR, —S(O)₂R, —S(O)₂N(R)₂, C₂₋₆ alkenyl, C₃₋₆ carbocycle, or heterocycle;
each R is independently a C₁₋₃ alkyl, C₂₋₃ alkenyl, or H;
each R′ is independently a C₁₋₁₈ alkyl, C₂₋₁₈ alkenyl, —R*YR″, —YR″, or H;
each R″ is independently a C₃₋₁₄ alkyl or C₃₋₁₄ alkenyl;
each R* is independently a C₁₋₁₂ alkyl or C₂₋₁₂ alkenyl;
each Y is independently a C₃₋₆ carbocycle;
each X is independently a F, Cl, Br, or I; and
m is 5, 6, 7, 8, 9, 10, 11, 12, or 13, and/or pharmaceutically acceptable salts, tautomers, prodrugs, or stereoisomers thereof.

In some aspects, another subset of compounds of Formula (I) includes those in which: R₁ is a C₅₋₃₀ alkyl, C₅₋₂₀ alkenyl, —R*YR″, —YR″, or —R″M′R′;
R₂ and R₃ are independently an H, C₁₋₁₄ alkyl, C₂₋₁₄ alkenyl, —R*YR″, —YR″, or —R*OR″, and/or R₂ and R₃, together with the atom to which they are attached, form a heterocycle or carbocycle;
R₄ is a C₃₋₆ carbocycle, —(CH₂)ₙQ, —(CH₂)ₙCHQR, —CHQR, —CQ(R)₂, or unsubstituted C₁₋₆ alkyl, where Q is a C₃₋₆ carbocycle, a 5- to 14-membered heteroaryl having one or more heteroatoms comprising N, O, or S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR) S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R) OR, or —C(═NR₉)N(R)₂, and/or each n is independently 1, 2, 3, 4, or 5;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′) C(O)—, —C(O)—, —C(S)—, —C(S)S—, —SC(S)—, —CH (OH)—, —P(O)(OR′)O—, —S(O)₂—, —S—S—, an aryl group, or a heteroaryl group;
R₇ is a C₁₋₃ alkyl, C₂₋₃ alkenyl, or H;
R₈ is a C₃₋₆ carbocycle or heterocycle;
R₉ is an H, CN, NO₂, C₁₋₆ alkyl, —OR, —S(O)₂R, —S(O)₂N(R)₂, C₂₋₆ alkenyl, C₃₋₆ carbocycle, or heterocycle;
each R is independently a C₁₋₃ alkyl, C₂₋₃ alkenyl, or H;
each R′ is independently a C₁₋₁₈ alkyl, C₂₋₁₈ alkenyl, —R*YR″, —YR″, or H;
each R″ is independently a C₃₋₁₄ alkyl or C₃₋₁₄ alkenyl;
each R* is independently a C₁₋₁₂ alkyl or C₂₋₁₂ alkenyl;
each Y is independently a C₃₋₆ carbocycle;
each X is independently a F, Cl, Br, or I; and
m is 5, 6, 7, 8, 9, 10, 11, 12, or 13, and/or pharmaceutically acceptable salts, tautomers, prodrugs, or stereoisomers thereof.

In some aspects, another subset of compounds of Formula (I) includes those in which:
R₁ is a C₅₋₃₀ alkyl, C₅₋₂₀ alkenyl, —R*YR″, —YR″, or —R″M′R′;
R₂ and R₃ are independently an H, C₂₋₁₄ alkyl, C₂₋₁₄ alkenyl, —R*YR″, —YR″, or —R*OR″, and/or R2 and R3, together with the atom to which they are attached, form a heterocycle or carbocycle;
R₄ is —(CH₂)ₙQ or —(CH₂)ₙCHQR, where Q is —N(R)₂, and/or n is 3, 4, or 5;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′) C(O)—, —C(O)—, —C(S)—, —C(S)S—, —SC(S)—, —CH (OH)—, —P(O)(OR′)O—, —S(O)₂—, —S—S—, an aryl group, or a heteroaryl group;
R₇ is a C₁₋₃ alkyl, C₂₋₃ alkenyl, or H;
each R is independently a C₁₋₃ alkyl, C₂₋₃ alkenyl, or H;
each R′ is independently a C₁₋₁₈ alkyl, C₂₋₁₈ alkenyl, —R*YR″, —YR″, or H;
each R″ is independently a C₃₋₁₄ alkyl or C₃₋₁₄ alkenyl;
each R* is independently a C₁₋₁₂ alkyl or C₁₋₁₂ alkenyl;
each Y is independently a C₃₋₆ carbocycle;
each X is independently a F, Cl, Br, or I; and
m is 5, 6, 7, 8, 9, 10, 11, 12, or 13, and/or pharmaceutically acceptable salts, tautomers, prodrugs, or stereoisomers thereof.

In some aspects, another subset of compounds of Formula (I) includes those in which:
R₁ is a C₅₋₃₀ alkyl, C₅₋₂₀ alkenyl, —R*YR″, —YR″, or —R″M′R′;
R₂ and R₃ are independently a C₁₋₁₄ alkyl, C₂₋₁₄ alkenyl, —R*YR″, —YR″, or —R*OR″, and/or R2 and R3, together with the atom to which they are attached, form a heterocycle or carbocycle;
R₄ is a-(CH₂)ₙQ, —(CH₂)ₙCHQR, —CHQR, or —CQ(R)₂, where Q is —N(R)₂, and/or n is 1, 2, 3, 4, or 5;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′) C(O)—, —C(O)—, —C(S)—, —C(S)S—, —SC(S)—, —CH (OH)—, —P(O)(OR′)O—, —S(O)₂—, —S—S—, an aryl group, or a heteroaryl group;
R₇ is a C₁₋₃ alkyl, C₂₋₃ alkenyl, or H;
each R is independently a C₁₋₃ alkyl, C₂₋₃ alkenyl, or H;
each R′ is independently a C₁₋₁₈ alkyl, C₂₋₁₈ alkenyl, —R*YR″, —YR″, or H;
each R″ is independently a C₃₋₁₄ alkyl or C₃₋₁₄ alkenyl;
each R* is independently a C₁₋₁₂ alkyl or C₁₋₁₂ alkenyl;
each Y is independently a C₃₋₆ carbocycle;
each X is independently a F, Cl, Br, or I; and
m is 5, 6, 7, 8, 9, 10, 11, 12, or 13, and/or pharmaceutically acceptable salts, tautomers, prodrugs, or stereoisomers thereof.

In some aspects, a subset of compounds of Formula (I) includes those of Formula (IA):

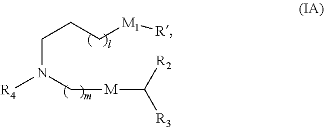

or a pharmaceutically acceptable salt, tautomer, prodrug, or stereoisomer thereof, wherein I is 1, 2, 3, 4, or 5; m is 5, 6, 7, 8, or 9; M1 is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently a —C(O)O—, —OC(O)—, —C(O)N(R′)—, —P(O)(OR′)O—, —S—S—, an aryl group, or a heteroaryl group; and R₂ and R₃ are independently a H, C₁₋₁₄ alkyl, or C₂₋₁₄ alkenyl.

In some aspects, a subset of compounds of Formula (I) includes those of Formula (II):

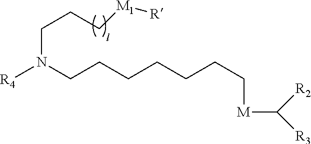

or a pharmaceutically acceptable salt, tautomer, prodrug, or stereoisomer thereof, wherein I is 1, 2, 3, 4, or 5; M1 is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently a —C(O)O—, —OC(O)—, —C(O)N(R′)—, —P(O)(OR′)O—, —S—S—, an aryl group, or a heteroaryl group; and R₂ and R₃ are independently a H, C₁₋₁₄ alkyl, or C₂₋₁₄ alkenyl. In some aspects, a subset of compounds of Formula (I) includes those of Formula (IIa), (IIb), (IIc), or (IIe):

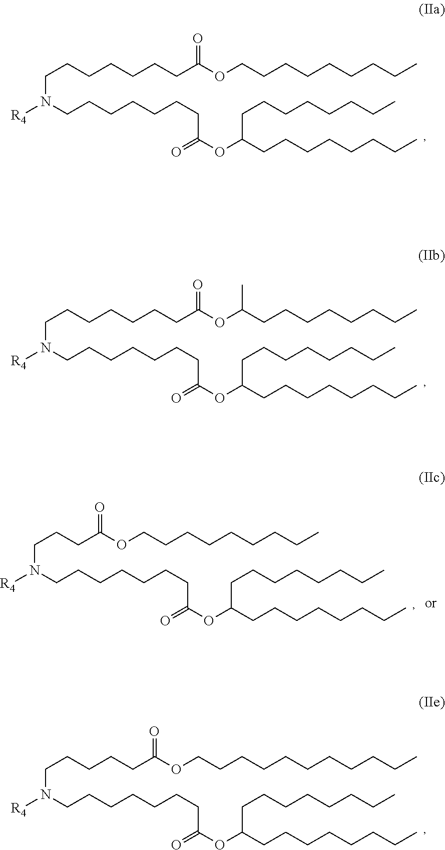

or a pharmaceutically acceptable salt, tautomer, prodrug, or stereoisomer thereof, wherein R₄ is as described herein.

In some aspects, a subset of compounds of Formula (I) includes those of Formula (IId):

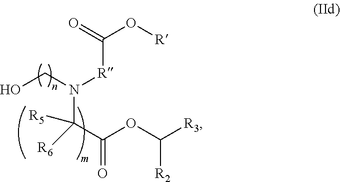

or a pharmaceutically acceptable salt, tautomer, prodrug, or stereoisomer thereof, wherein n is 2, 3, or 4; and m, R′, R″, and R₂ through Re are as described herein. For example, each of R₂ and R₃ may be independently a C₅₋₁₄ alkyl or C₅₋₁₄ alkenyl.

In some aspects, an ionizable cationic lipid of the disclosure comprises a compound having structure:

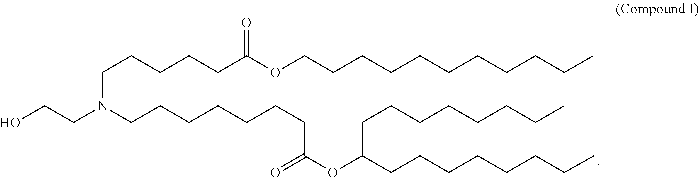 [SMILES: CCCCCCCCCCCOC(=O)CCCCCN(CCO)CCCCCCCC(=O)OC(CCCCCCCC)CCCCCCCC]

In some aspects, an ionizable cationic lipid of the disclosure comprises a compound having structure:

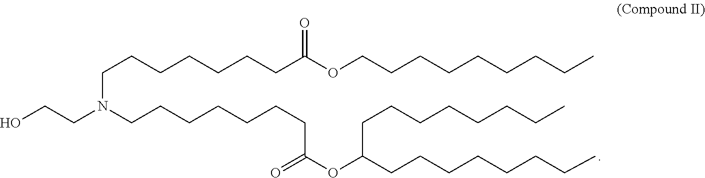 [SMILES: CCCCCCCCCOC(=O)CCCCCCCN(CCO)CCCCCCCC(=O)OC(CCCCCCCC)CCCCCCCC]

LNPs described herein can also be generated using the cationic lipids and compositions comprising them as are known in the art, see, e.g., PCT Publication Nos. WO2015/199952, WO2017/004143, WO2017/075531, WO2017/117528, WO2016/176330, WO2018/191657, WO2018/081480, WO2018/107026, WO2018/200943, WO2018/078053, WO2019/036000, WO2019/036028, WO2019/036030, WO2019/036008, WO2020/061426, WO2020/081938, WO2020/146805, WO2021/030701, WO2022/016070, WO2023/114944, WO2023/114937, WO2023/114943, WO2024/054843, and WO2023/250427, all of which are incorporated by reference herein in their entirety for all purposes.

In some aspects, an ionizable cationic lipid of the disclosure comprises a compound having structure:

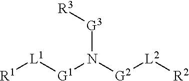

or a pharmaceutically acceptable salt, tautomer, prodrug, or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)═NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or ª direct bond;
G¹ and G² are each independently unsubstituted C₁-C₁₂ alkylene or C₁-C₁₂ alkenylene; G is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C24 alkyl or C₆-C₂₄ alkenyl;
R₃ is H, OR⁵, CN, —C(═O)OR⁴, —OC(═O)R⁴ or —NR⁵C(═O)R⁴;
R₄ is C₁-C12 alkyl;
R₅ is H or C₁-C₆ alkyl; and
x is 0, 1, or 2.

In some of the foregoing aspects, the ionizable cationic lipid comprises a compound having one of the following structures:

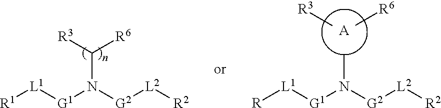

or a pharmaceutically acceptable salt, tautomer, prodrug, or stereoisomer thereof, wherein:
A is a 3 to 8-membered cycloalkyl or cycloalkylene ring;
R⁶ is, at each occurrence, independently H, OH or C₁-C₂₄ alkyl; and n is an integer ranging from 1 to 15.

In some of the foregoing aspects, the ionizable cationic lipid comprises a compound having one of the following structures:

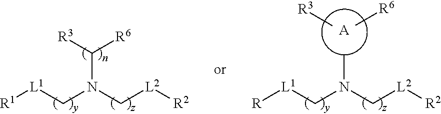

or a pharmaceutically acceptable salt, tautomer, prodrug, or stereoisomer thereof, wherein y and z are each independently integers ranging from 1 to 12.

In any of the foregoing aspects, one of L¹ or L² is —OCCO)—. For example, in some aspects, each of L¹ and L² are —O(C═O)—. In some aspects of any of the foregoing, L¹ and L² are each independently —(C═O)O— or —O(C═O)—. For example, in some aspects, each of L¹ and L² is —(C═O)O—.

In some of the foregoing aspects, the ionizable cationic lipid comprises a compound having one of the following structures:

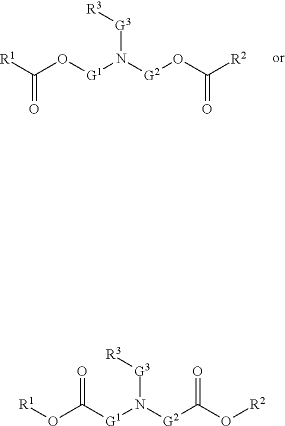

or a pharmaceutically acceptable salt, tautomer, prodrug, or stereoisomer thereof.

In some of the foregoing aspects, the ionizable cationic lipid comprises a compound having one of the following structures:

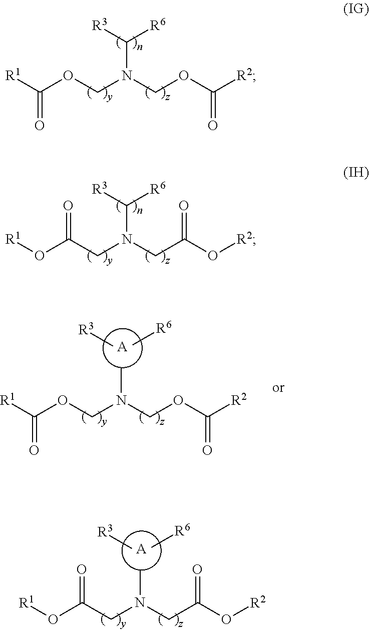

Or a pharmaceutically acceptable salt, tautomer, prodrug, or stereoisomer thereof.

In some of the foregoing aspects, n is an integer ranging from 2 to 12, for example from 2 to 8 or from 2 to 4. For example, in some aspects, n is 3, 4, 5, or 6. In some aspects, n is 3. In some aspects, n is 4. In some aspects, n is 5. In some aspects, n is 6.

In some of the foregoing aspects, y and z are each independently an integer ranging from 2 to 10. For example, in some aspects, y and z are each independently an integer ranging from 4 to 9 or from 4 to 6.

In some of the foregoing aspects, R⁶ is H. In other of the foregoing embodiments, R⁶ is C₁-C₂₄ alkyl. In other aspects, R⁶ is OH. In some embodiments, G is unsubstituted. In other aspects, G³ is substituted. In various different aspects, G³ is linear C₁-C₂₄ alkylene or linear C1-C24 alkenylene.

In some other foregoing embodiments, R¹ or R², or both, is C₆-C₂₄ alkenyl. For example, in some embodiments, R¹ and R² each, independently have the following structure:

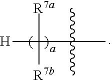

wherein:
R^{7a} and R^{7b} are, at each occurrence, independently H or C₁-C₁₂ alkyl; and a is an integer from 2 to 12,
wherein R^{7a}, R^{7b}, and a are each selected such that R¹ and R² each independently comprise from 6 to 20 carbon atoms. For example, in some embodiments a is an integer ranging from 5 to 9 or from 8 to 12.

In some of the foregoing aspects, at least one occurrence of R^{7a} is H. For example, in some aspects, R^{7a} is H at each occurrence. In other different aspects of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl, or n-octyl.

In different aspects. R¹ or R², or both, has one of the following:

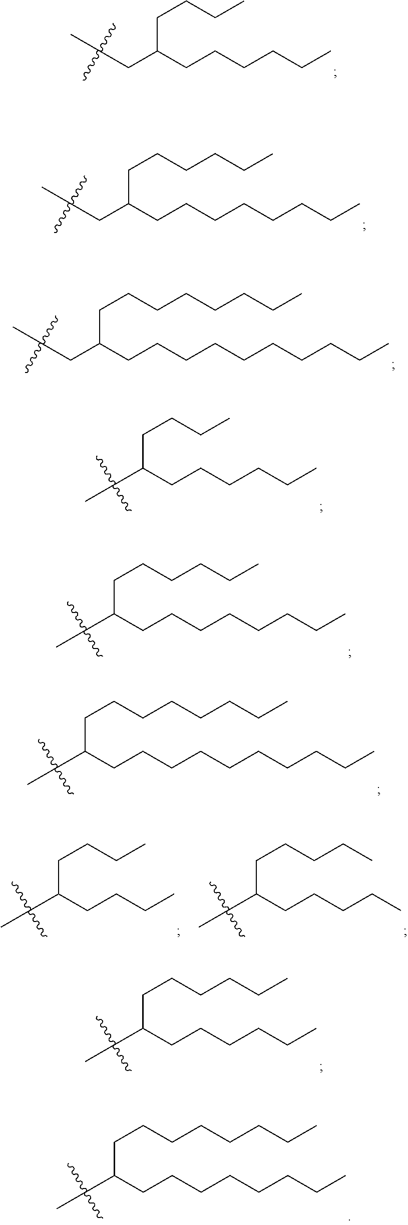

In some of the foregoing aspects, R is OH, CN, —C(═O)OR⁴—OC(═O)R⁴ or —NHC(═O)R⁴. In some aspects, R⁴ is methyl or ethyl.

It is understood that any aspect of the compounds set forth above, and any specific substituent and/or variable in the compounds set forth above, may be independently combined with other aspects and/or substituents and/or variables of compounds to form aspects of the inventions not specifically set forth above. In addition, in the event that a list of substituents and/or variables is listed for any particular substituent and/or variable in a particular embodiment and/or claim, it is understood that each individual substituent and/or variable may be deleted from the particular aspect and/or claim and that the remaining list of substituents and/or variables will be considered to be within the scope of the disclosure. It is understood that in the present description, combinations of substituents and/or variables of the depicted formulae are permissible only if such contributions result in stable compounds.

In some embodiments, the cationic lipid is

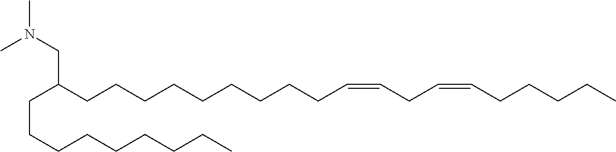 [SMILES: CCCCC/C=C\C/C=C\CCCCCCCCCC(CCCCCCCCC)CN(C)C]

In some embodiments, the cationic lipid is

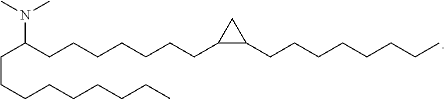 [SMILES: CCCCCCCCCC(CCCCCCCC1CC1CCCCCCCC)N(C)C]

In some aspects, the lipid nanoparticles comprise one or more cationic lipids. In one aspect, the lipid nanoparticles comprise (4-hydroxybutyl)azanediyl)bis(hexane-6,1-diyl)bis(2-hexyldecanoate) (ALC-0315), having the formula:

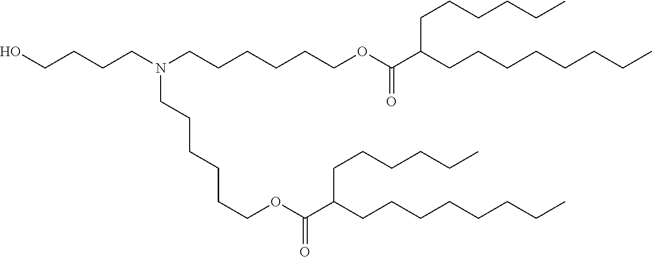 [SMILES: CCCCCCCCC(CCCCCC)C(=O)OCCCCCCN(CCCCO)CCCCCCOC(=O)C(CCCCCC)CCCCCCCC]

Exemplary cationic lipids are disclosed in, e.g., U.S. Pat. No. 10,166,298, the full disclosures of which are herein incorporated by reference in their entirety for all purposes. Representative cationic lipids are set forth in Table 23:

TABLE 23

| No. | Structure |
|---|---|
| 1 | 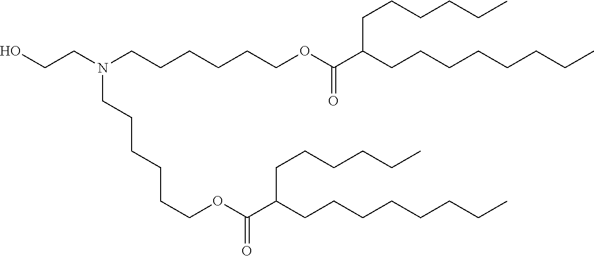 [SMILES: CCCCCCCCC(CCCCCC)C(=O)OCCCCCCN(CCO)CCCCCCOC(=O)C(CCCCCC)CCCCCCCC] |
| 2 | 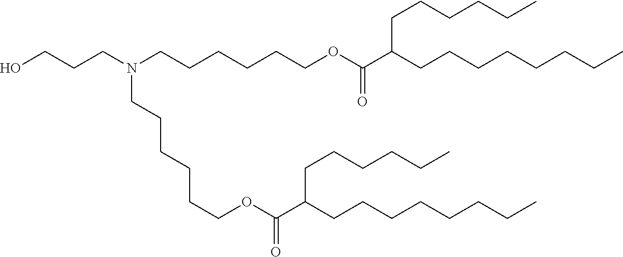 [SMILES: CCCCCCCCC(CCCCCC)C(=O)OCCCCCCN(CCCO)CCCCCCOC(=O)C(CCCCCC)CCCCCCCC] |
| 3 | 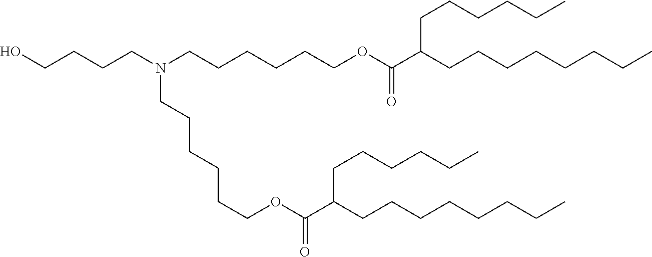 [SMILES: CCCCCCCCC(CCCCCC)C(=O)OCCCCCCN(CCCCO)CCCCCCOC(=O)C(CCCCCC)CCCCCCCC] |
| 4 | 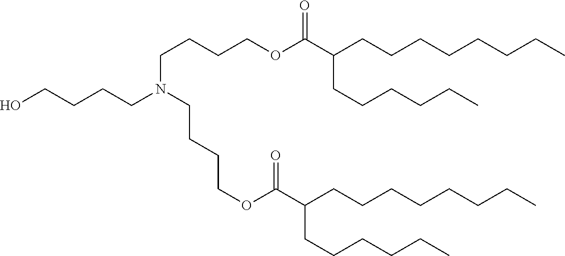 [SMILES: CCCCCCCCC(CCCCCC)C(=O)OCCCCN(CCCCO)CCCCOC(=O)C(CCCCCC)CCCCCCCC] |
| 5 | 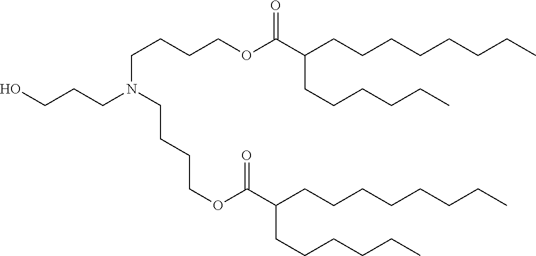 [SMILES: CCCCCCCCC(CCCCCC)C(=O)OCCCCN(CCCO)CCCCOC(=O)C(CCCCCC)CCCCCCCC] |
| 6 | 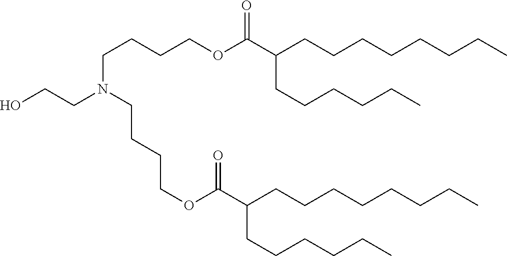 [SMILES: CCCCCCCCC(CCCCCC)C(=O)OCCCCN(CCO)CCCCOC(=O)C(CCCCCC)CCCCCCCC] |
| 7 | 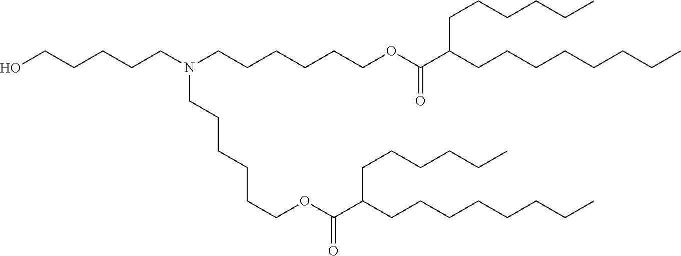 [SMILES: CCCCCCCCC(CCCCCC)C(=O)OCCCCCCN(CCCCCO)CCCCCCOC(=O)C(CCCCCC)CCCCCCCC] |
| 8 | 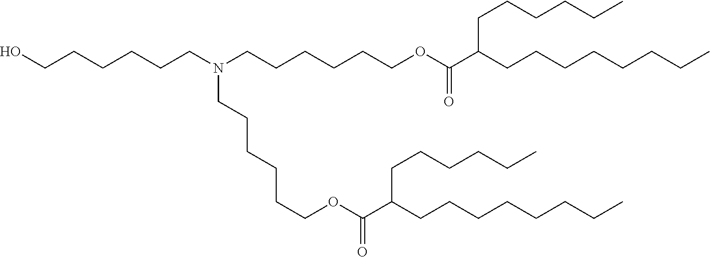 [SMILES: CCCCCCCCC(CCCCCC)C(=O)OCCCCCCN(CCCCCCO)CCCCCCOC(=O)C(CCCCCC)CCCCCCCC] |
| 9 | 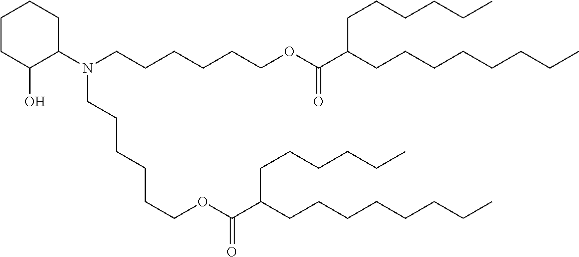 [SMILES: CCCCCCCCC(CCCCCC)C(=O)OCCCCCCN(CCCCCCOC(=O)C(CCCCCC)CCCCCCCC)C1CCCCC1O] |
| 10 | 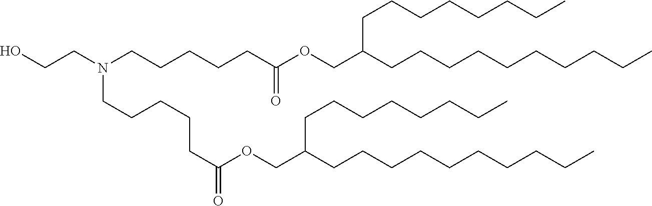 [SMILES: CCCCCCCCCCC(CCCCCCCC)COC(=O)CCCCCN(CCO)CCCCCC(=O)OCC(CCCCCCCC)CCCCCCCCCC] |
| 11 | 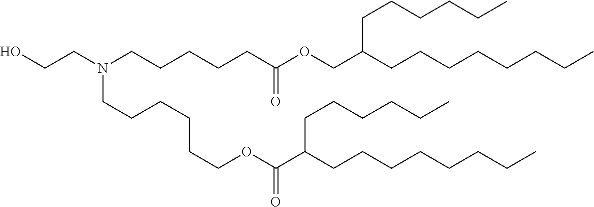 [SMILES: CCCCCCCCC(CCCCCC)COC(=O)CCCCCN(CCO)CCCCCCOC(=O)C(CCCCCC)CCCCCCCC] |
| 12 | 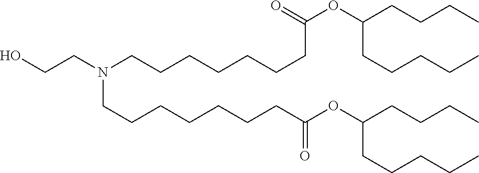 [SMILES: CCCCCC(CCCC)OC(=O)CCCCCCCN(CCO)CCCCCCCC(=O)OC(CCCC)CCCCC] |
| 13 | 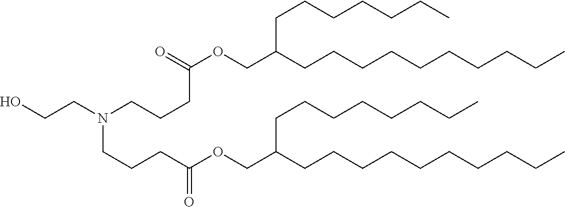 [SMILES: CCCCCCCCCCC(CCCCCCC)COC(=O)CCCN(CCO)CCCC(=O)OCC(CCCCCCCC)CCCCCCCCCC] |
| 14 | 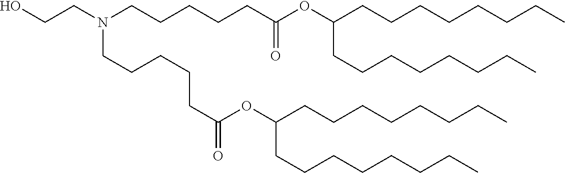 [SMILES: CCCCCCCCC(CCCCCCCC)OC(=O)CCCCCN(CCO)CCCCCC(=O)OC(CCCCCCCC)CCCCCCCC] |
| 15 | 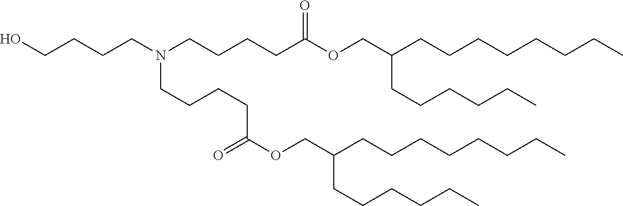 [SMILES: CCCCCCCCC(CCCCCC)COC(=O)CCCCN(CCCCO)CCCCC(=O)OCC(CCCCCC)CCCCCCCC] |
| 16 | 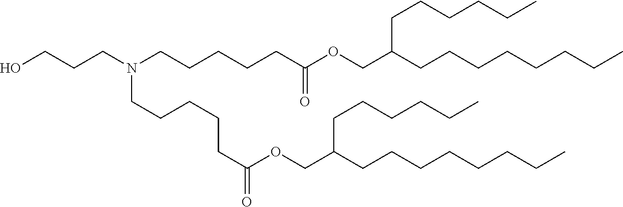 [SMILES: CCCCCCCCC(CCCCCC)COC(=O)CCCCCN(CCCO)CCCCCC(=O)OCC(CCCCCC)CCCCCCCC] |
| 17 | 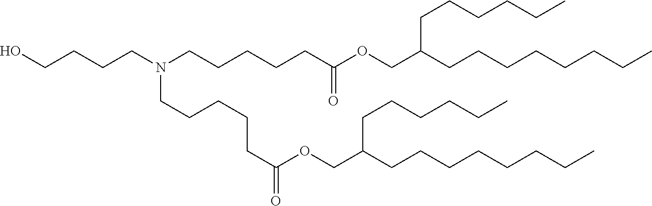 [SMILES: CCCCCCCCC(CCCCCC)COC(=O)CCCCCN(CCCCO)CCCCCC(=O)OCC(CCCCCC)CCCCCCCC] |
| 18 | 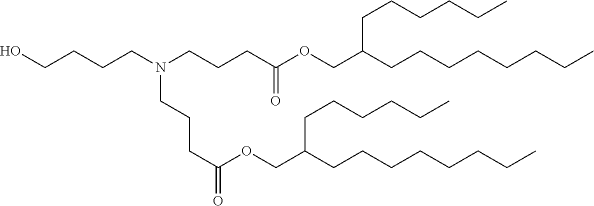 [SMILES: CCCCCCCCC(CCCCCC)COC(=O)CCCN(CCCCO)CCCC(=O)OCC(CCCCCC)CCCCCCCC] |
| 19 | 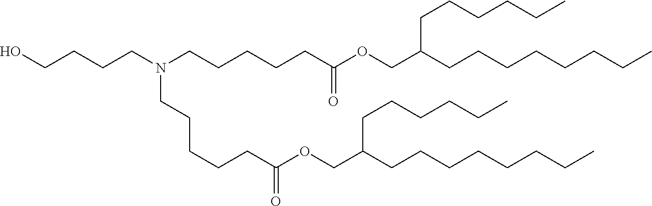 [SMILES: CCCCCCCCC(CCCCCC)COC(=O)CCCCCN(CCCCO)CCCCCC(=O)OCC(CCCCCC)CCCCCCCC] |
| 20 | 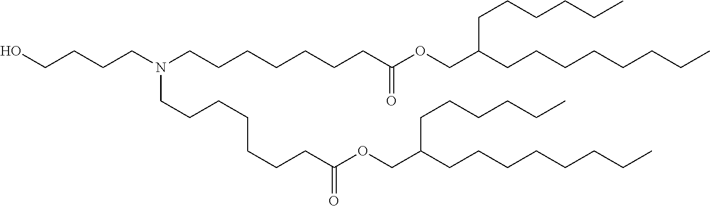 [SMILES: CCCCCCCCC(CCCCCC)COC(=O)CCCCCCCN(CCCCO)CCCCCCCC(=O)OCC(CCCCCC)CCCCCCCC] |
| 21 | 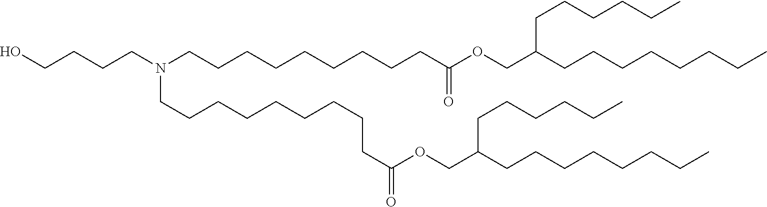 [SMILES: CCCCCCCCC(CCCCCC)COC(=O)CCCCCCCCCN(CCCCO)CCCCCCCCCC(=O)OCC(CCCCCC)CCCCCCCC] |
| 22 | 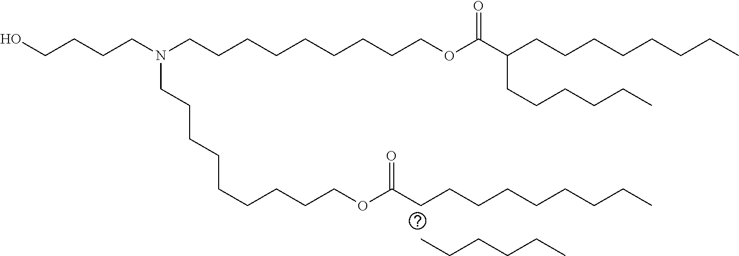 |
| 23 | 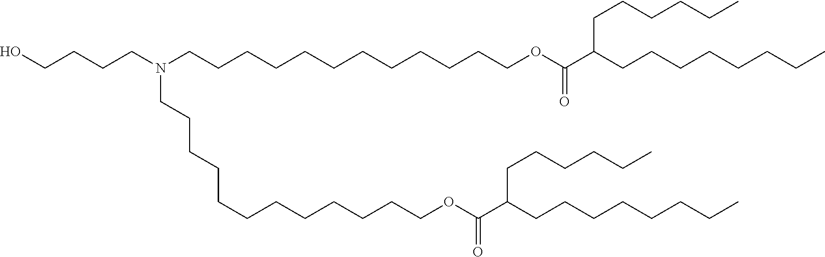 [SMILES: CCCCCCCCC(CCCCCC)C(=O)OCCCCCCCCCCCCN(CCCCO)CCCCCCCCCCCCOC(=O)C(CCCCCC)CCCCCCCC] |
| 24 | 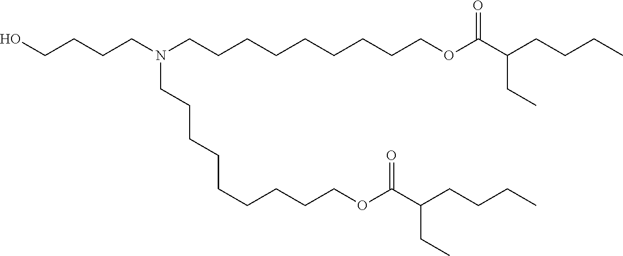 [SMILES: CCCCC(CC)C(=O)OCCCCCCCCCN(CCCCO)CCCCCCCCCOC(=O)C(CC)CCCC] |
| 25 | 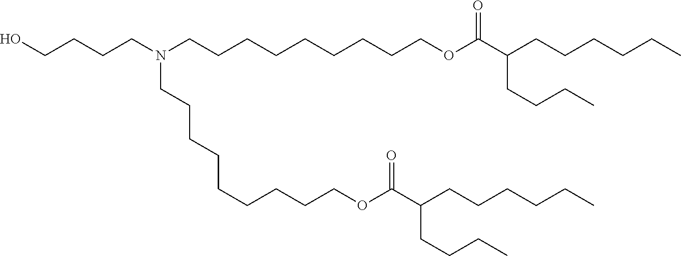 [SMILES: CCCCCCC(CCCC)C(=O)OCCCCCCCCCN(CCCCO)CCCCCCCCCOC(=O)C(CCCC)CCCCCC] |
| 26 | 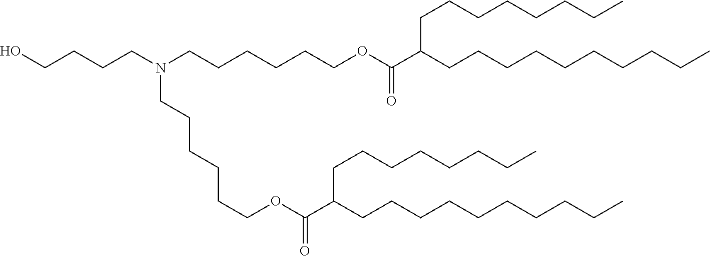 [SMILES: CCCCCCCCCCC(CCCCCCCC)C(=O)OCCCCCCN(CCCCO)CCCCCCOC(=O)C(CCCCCCCC)CCCCCCCCCC] |
| 27 | 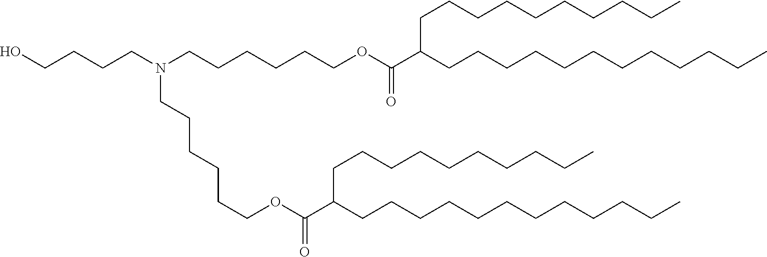 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| 28 | 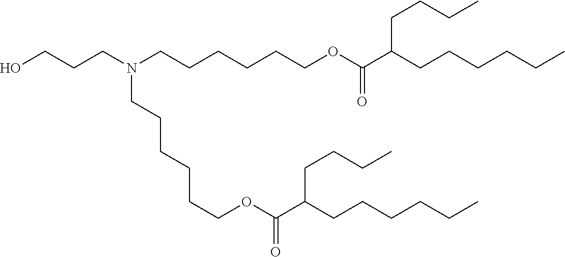 [SMILES: CCCCCCC(CCCC)C(=O)OCCCCCCN(CCCO)CCCCCCOC(=O)C(CCCC)CCCCCC] |
| 29 | 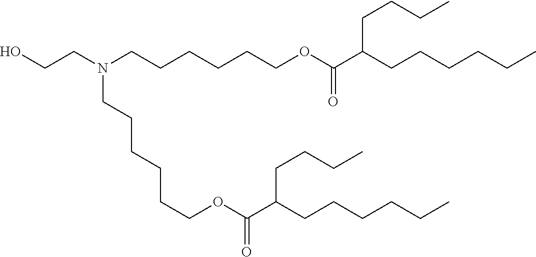 [SMILES: CCCCCCC(CCCC)C(=O)OCCCCCCN(CCO)CCCCCCOC(=O)C(CCCC)CCCCCC] |
| 30 | 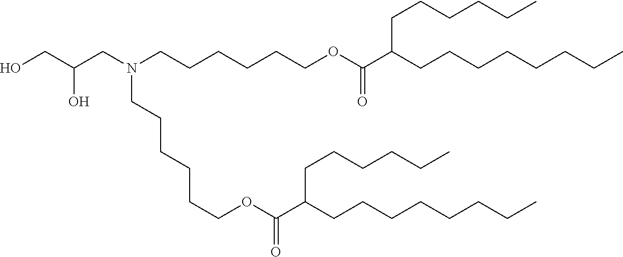 [SMILES: CCCCCCCCC(CCCCCC)C(=O)OCCCCCCN(CCCCCCOC(=O)C(CCCCCC)CCCCCCCC)CC(O)CO] |
| 31 | 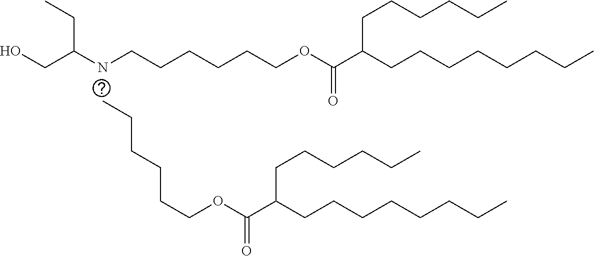 |
| 32 | 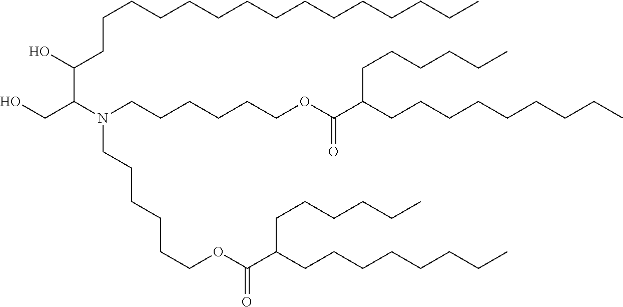 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCCC] |
| 33 | 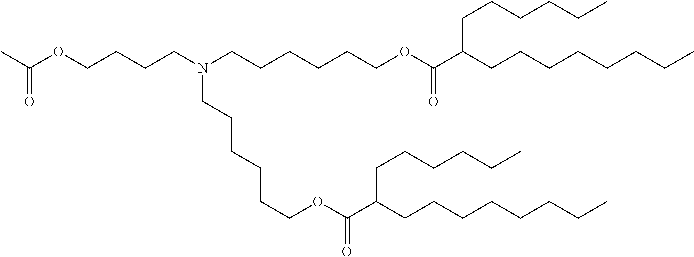 [SMILES: CCCCCCCCC(CCCCCC)C(=O)OCCCCCCN(CCCCCCOC(=O)C(CCCCCC)CCCCCCCC)CCCCOC(C)=O] |
| 34 | 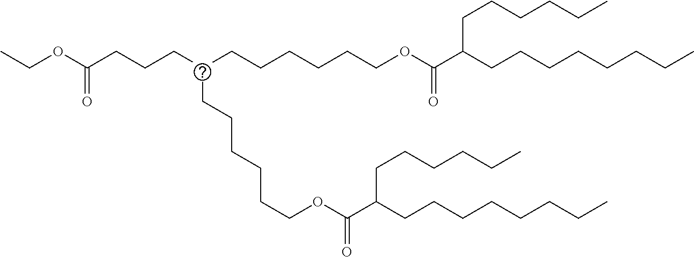 [SMILES: CCCCCCCCC(CCCCCC)C(=O)OCCCCCCC(CCCCCCOC(=O)C(CCCCCC)CCCCCCCC)CCCC(=O)OCC] |
| 35 | 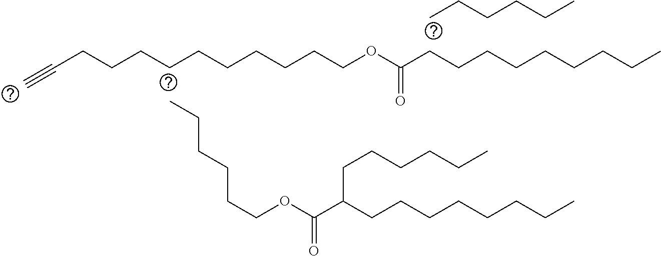 |
| 36 | 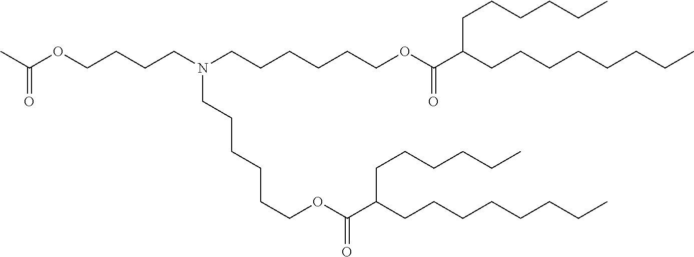 [SMILES: CCCCCCCCC(CCCCCC)C(=O)OCCCCCCN(CCCCCCOC(=O)C(CCCCCC)CCCCCCCC)CCCCOC(C)=O] |

In yet another aspect, the ionizable cationic lipid is described in PCT Publication No. WO2015/199952, the contents of which is hereby incorporated herein by reference in its entirety for all purposes. In one embodiment therein, an ionizable cationic lipid of the disclosure comprises a compound of Formula (I):

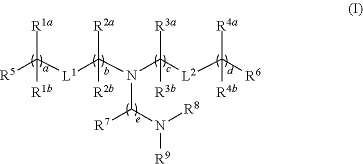

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2.

In other embodiments, the lipid compounds have the following Structure (Ia):

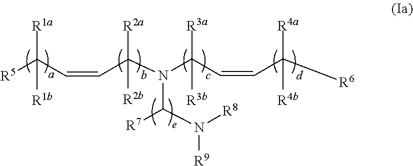

In another embodiment, the lipid compounds have the following Structure (Ib):

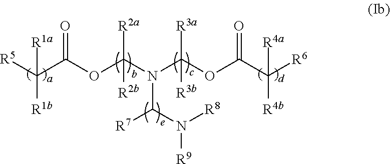

In yet other embodiments, the lipid compounds have the following Structure (Ic):

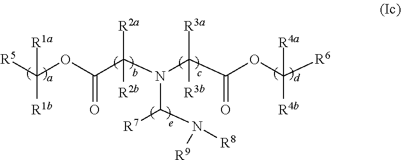

Compound Nos. 1-41 as specifically exemplified in Table 1 of PCT Publication No. WO2015/199952, and falling within the generic scope of Structures (I), (Ia), (Ib) or (Ic) described above, are hereby incorporated herein by reference for all purposes.

In yet another aspect, the ionizable cationic lipid is described in PCT Publication No. WO2016/176330, the contents of which is hereby incorporated herein by reference in its entirety for all purposes. In one embodiment therein, an ionizable cationic lipid of the disclosure comprises a compound of Formula (I):

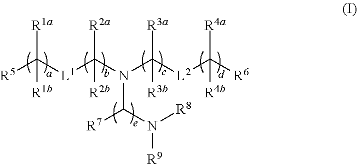

or a pharmaceutically acceptable salt, tautomer, prodrug or stereoisomer thereof, wherein:
L¹ and L² are each independently —O(C═O)—, —(C═O)O— or a carbon carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and e is 1 or 2.

In one embodiment, the ionizable cationic lipid comprises a compound having a structure of Formula (II):

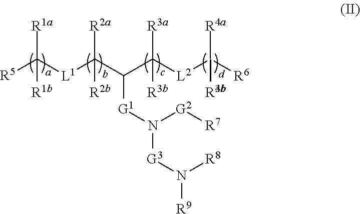

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}, —OC(═O)NR^{a}—, —NR^{a}C(═O)O—, or a direct bond;
G¹ is C₁-C2 alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a} or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C12 alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

In one embodiment, the ionizable cationic lipid comprises a compound having a structure of Formula (III):

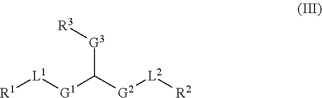

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C24 alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C24 alkyl or C₆-C₂₄ alkenyl; R³ is H, OR⁵, CN, —C(═O)OR⁴, —OC(═O)R⁴ or —NR⁵C(═O)R⁴;
R⁴ is C₁-C12 alkyl;
R⁵ is H or C₁-C₆ alkyl; and
x is 0, 1 or 2.

Compound Nos. I-1 to I-41 as specifically exemplified in Table 1, Compound Nos. II-1 to II-34 as specifically exemplified in Table 2, and Compound Nos. III-1 to III-36 as specifically exemplified in Table 3, of PCT Publication No. WO2016/176330, and falling within the generic scope of Structures (I), (II) or (III) described above, are hereby incorporated herein by reference for all purposes.

In yet another aspect, the ionizable cationic lipid is described in PCT Publication No. WO2017/004143, the contents of which is hereby incorporated herein by reference in its entirety for all purposes. In one embodiment therein, an ionizable cationic lipid of the disclosure comprises a compound of Formula (I):

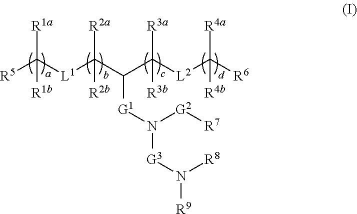

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)R^{a}—, —NR^{a}C(═O)O— or a direct bond;
G¹ is C₁-C2 alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C12 alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

In one embodiment, the cationic lipid comprises a compound having a structure of Formula (IA), (IB), (IC) or (ID):

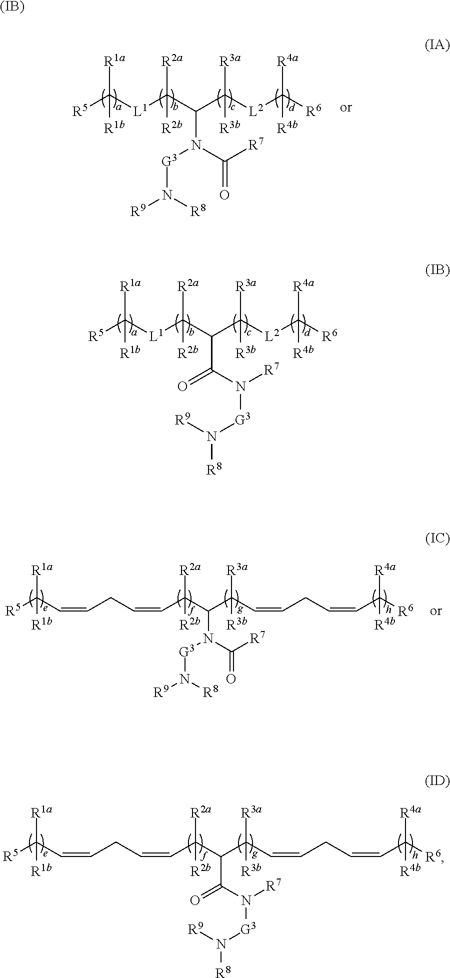

wherein e, f, g and h are each independently an integer from 1 to 12.

In different aspects, RD is branched C₁-C₁₅ alkyl. For example, in some embodiments R′ has one of the following structures:

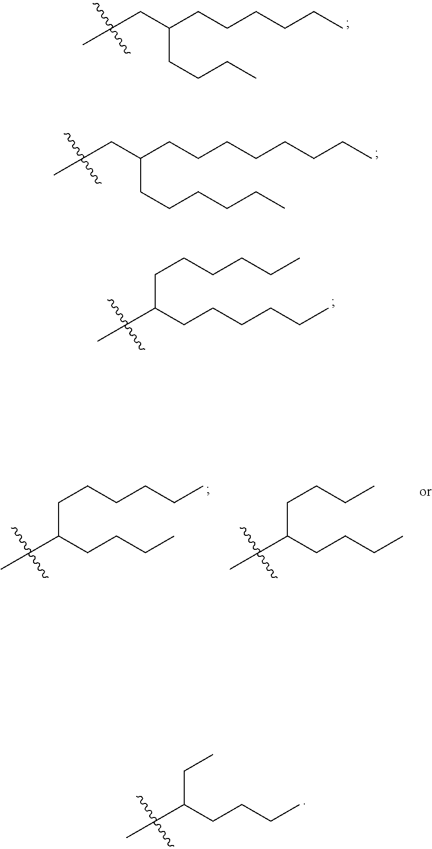

Compound Nos. 1-46 as specifically exemplified in Table 1 of PCT Publication No. WO2017/004143 described above, are hereby incorporated herein by reference for all purposes.

In another aspect, the cationic lipid is described in PCT Publication No. WO2017/117528, the contents of which is hereby incorporated herein by reference in its entirety for all purposes. In one embodiment therein, a cationic lipid of the disclosure comprises a compound of Formula (I):

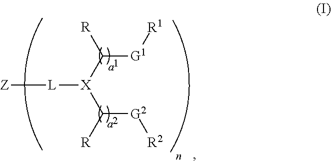

or a pharmaceutically acceptable salt, prodrug or stereoisomer thereof.

Compound Nos. 1-3 as specifically exemplified in Table 1 of PCT Publication No. WO2017/117528 described above, are hereby incorporated herein by reference for all purposes.

In another aspect, the cationic lipid is described in PCT Publication No. WO2018/191657, the contents of which is hereby incorporated herein by reference in its entirety for all purposes. In one embodiment therein, a cationic lipid of the disclosure comprises a compound of Formula (I):

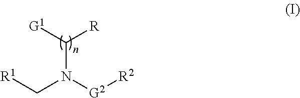

or a pharmaceutically acceptable salt, prodrug or stereoisomer thereof, wherein:
G¹ is —OH, —R³R⁴, —(C═O)NR⁵ or —NR³ (C═O)R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and n is an integer from 2 to 6.

In one embodiment, the cationic lipid comprises a compound having a structure of Formula (IA):

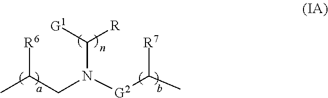

wherein:
R⁶ and R⁷ are, at each occurrence, independently H or straight or branched, saturated or unsaturated C₁-C₁₄ alkyl;
a and b are each independently an integer ranging from 1 to 15, provided that Re and a, and R⁷ and b, are each independently selected such that R¹ and R², respectively, are each independently branched, saturated or unsaturated C₁₂-C₃₆ alkyl.

In one embodiment, the cationic lipid comprises a compound having a structure of Formula (IB):

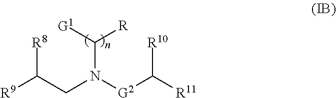

wherein R₈, R⁹, R¹⁰ and R¹¹ are each independently straight or branched, saturated or unsaturated C₄-C₁₂ alkyl, provided that R⁸ and R⁹, and R¹⁰ and R¹¹, are each independently selected such that R¹ and R², respectively, are each independently branched, saturated of unsaturated C₁₂-C₃₆ alkyl.

In different aspects, R¹ and R² have the following structure:

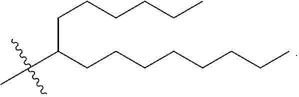 [SMILES: *C(CCCCCC)CCCCCCCC]

Compound Nos. 1-17 as specifically exemplified in Table 1 of PCT Publication No. WO2018/191657 described above, are hereby incorporated herein by reference for all purposes.

In yet another aspect, the cationic lipid is described in PCT Publication No. WO2018/081480, the contents of which is hereby incorporated herein by reference in its entirety for all purposes. In one embodiment therein, a cationic lipid of the disclosure comprises a compound of Structure (I), (II), (III), (IV) or (IV), or a pharmaceutically acceptable salt, prodrug or stereoisomer thereof.

Compound Nos. I-1 to I-41 as specifically exemplified in Table 1, Compound Nos. II-1 to II-46 as specifically exemplified in Table 2, Compound Nos. III-1 to III-49 as specifically exemplified in Table 3, and Compound Nos. IV-1 to IV-3 as specifically exemplified in Table 4 of PCT Publication No. WO2018/081480 described above, are hereby incorporated herein by reference for all purposes.

In yet another aspect, the cationic lipid is described in PCT Publication No. WO2018/107026, the contents of which is hereby incorporated herein by reference in its entirety for all purposes. In one embodiment therein, a cationic lipid of the disclosure is selected from compounds having the following Formulas (I, II, III and IV):

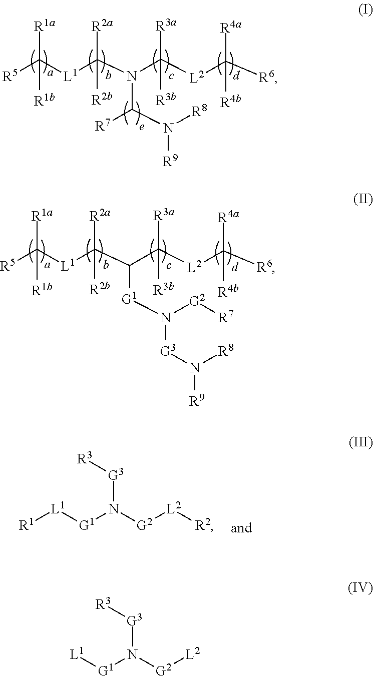

or a pharmaceutically acceptable salt, tautomer or stereoisomer thereof, wherein, for Formula (I):
L¹ and L² are each independently —O(C═O)—, (C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon 20 double bond;
R⁵ and R⁶ are each independently methyl or cycloalkyl; R⁷ is, at each occurrence, independently H or C₁-C₁₂ alkyl;
R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom; a and d are each independently an integer from 0 to 24; b and c are each independently an integer from 1 to 24; and e is 1 or 2, for Formula (II):
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C2 alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C1-C12 alkyl;
R^{1a} and R^{1b} are, at each occurrence, independently either: (a) H or C1-C12 alkyl; or (b) R^{1a} is H or C1-C12 alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C1-C12 alkyl; or (b) R^{2a} is H or C1-C12 alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either: (a) H or C1-C12 alkyl; or (b) R^{3a} is H or C1-C12 alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C1-C12 alkyl; or (b) R^{4a} is H or C1-C12 alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C4-C20 alkyl;
R⁸ and R⁹ are each independently C₁-C12 alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2;

### for Formula (III):

one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}—, —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)R^{a}—, —OC(═O)R^{a}— or —NR^{a}C(═O)O— or a direct bond;
G¹ and G² are each independently unsubstituted C1-C 12 alkylene or C1-C12 alkenylene;
G³ is C1-C24 alkylene, C1-C24 alkenylene, C3-C8 cycloalkylene, C3-C8 cycloalkenylene;
R^{a} is H or C1-C12 alkyl;
R¹ and R² are each independently C6-C24 alkyl or C6-C24 alkenyl;
R³ is H, OR⁵, CN, —C(═O)OR⁴, —OC(═O)R⁴ or —NR⁵C(═O)R⁴;
R⁴ is C1-C12 alkyl;
R⁵ is H or C1-C6 alkyl; and
x is 0, 1 or 2; and

### for Formula (IV):

one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}—, —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, —NR^{a}C(═O)NR^{a}—, —OC(═O)NR^{a}— or —NR^{a}C(═O)O— or a direct bond;
G¹ and G² are each independently unsubstituted C1-C12 alkylene or C1-C12 alkenylene;
G³ is C1-C24 alkylene, C1-C24 alkenylene, C3-C8 cycloalkylene, C3-C8 cycloalkenylene;
R^{a} is H or C1-C12 alkyl;
R¹ and R² are each independently C6-C24 alkyl or C6-C24 alkenyl;
R³ is H, OR⁵, CN, —C(═O)OR⁴, —OC(═O)R⁴ or —NR⁵C(═O)R⁴;
R⁴ is C1-C12 alkyl;
R⁵ is H or C1-C6 alkyl; and
x is 0, 1 or 2.

In one embodiment, the cationic lipid is selected from any one of Compound Nos. I-1 to I-41 as specifically exemplified in Table 1, Compound Nos. II-1 to II-36 as specifically exemplified in Table 2, Compound Nos. III-1 to III-49 as specifically exemplified in Table 3, and Compound Nos. 1 to 17 as specifically exemplified in Table 4, of PCT Publication No. WO2018/107026, which Tables and compounds listed therein are hereby incorporated herein by reference for all purposes.

In yet another aspect, the cationic lipid is described in PCT Publication No. WO2018/200943, the contents of which is hereby incorporated herein by reference in its entirety for all purposes. In one embodiment therein, a cationic lipid of the disclosure is selected from compounds having the following Structures (1) or (II):

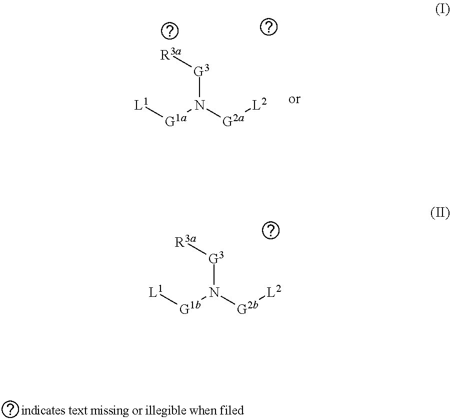

or a pharmaceutically acceptable salt, prodrug or stereoisomer thereof, wherein:
L¹ is —O(C═O)R¹, —(C═O)OR′, —C(═O)R¹, —OR′, —S(O)ₓR¹, —S—SR¹, —C(═O)SR¹, —SC(═O)R¹, —NR^{a}C(═O)R¹, —C(═O)NR^{b}R^{c}, —NR^{a}C(═O)R^{b}R^{c}, —OC(═O)NR^{b}R^{c} or —NR^{a}C(═O)OR¹;
L² is —O(C═O)R², —(C═O)OR², —C(═O)R, —OR², —S(O)ₓR², —S—SR², —C(═O)SR², —SC(═O)R², —NR^{a}C(═O)R², —C(═O)NR^{e}R^{f}, —NR^{d}C(═O)NR^{e}R^{f}, —OC(═O)NR^{e}R^{f}, —NR^{d}C(═O)OR² or a direct bond to R²;
G^{1a} and G^{2a} are each independently C2-C12 alkylene or C2-C12 alkenylene;
G^{1b} and G^{2b} are each independently C1-C12 alkylene or C₂-C₁₂ alkenylene;
G³ is C1-C24 alkylene, C2-C24 alkenylene, C3-C8 cycloalkylene or C₃-C₆ cycloalkenylene;
R^{a}, R^{b}, R^{d} and R^{e} are each independently H or C1-C12 alkyl or C2-C12 alkenyl;
R^{c} and R^{f} are each independently C1-C12 alkyl or C2-C12 alkenyl;
R¹ and R² are each independently branched C₆-C₂4 alkyl or branched C₆-C₂₄ alkenyl;
R^{3a} is —C(═O)N(R^{4a})R^{5a} or —C(═O)OR⁶;
R^{3b} is —NR^{4b}C(═O)R^{5b};
R^{4a} is C1-C12 alkyl;
R^{4b} is H, C1-C12 alkyl or C₂-C₁₂ alkenyl;
R^{5a} is H, C1-C8 alkyl or C₂-C₈ alkenyl;
R^{5b} is C2-C12 alkyl or C₂-C₁₂ alkenyl when R^{4b} is H; or R^{5b} is C1-C12 alkyl or C₂-C₁₂ alkenyl when R^{4b} is C1-C12 alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

In one embodiment, a cationic lipid of the disclosure is selected from compounds having the following Structures (IA) or (IIA):

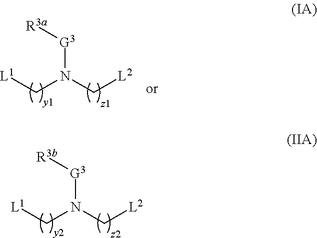

wherein y1 and z1 are each independently integers ranging from 2 to 12, and y2 and z2 are each independently integers ranging from 1 to 12.

In another embodiment, a cationic lipid of the disclosure is selected from compounds having the following Structures (IB), (IC), (ID), (IE), (IIB) or (IIC):

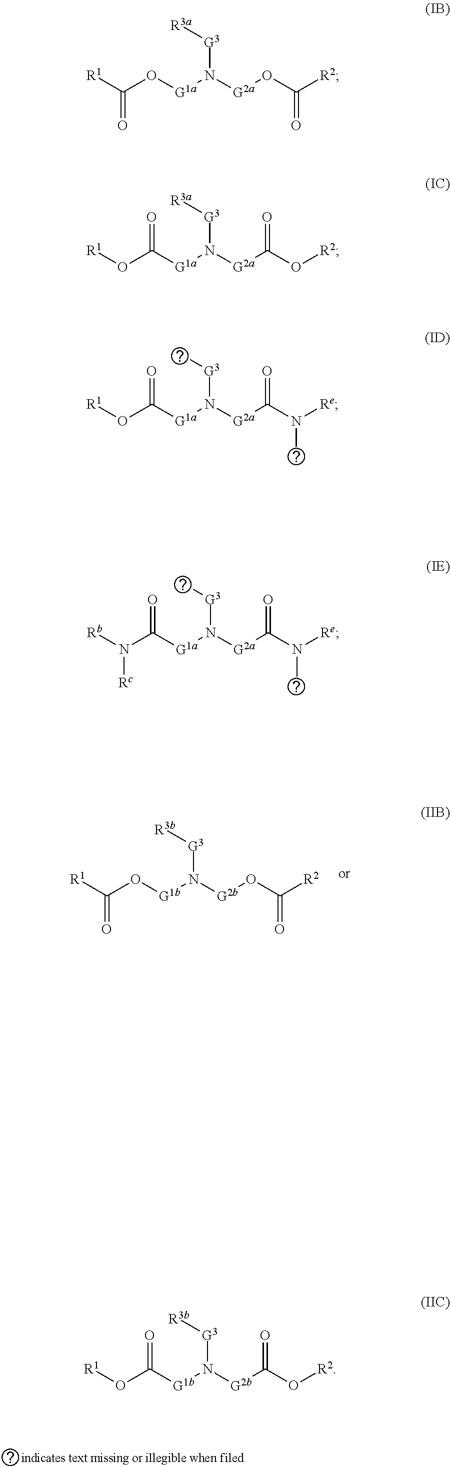

In another embodiment, a cationic lipid of the disclosure is selected from compounds having the following Structures (IF), (IG), (IH), (IJ), (IID) or (IIE):

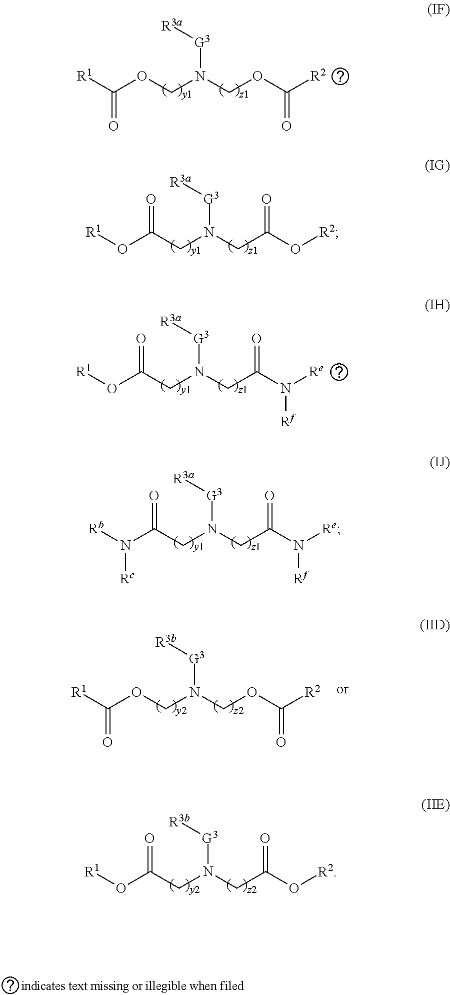

In different aspects, R¹ or R², or both, independently has one of the following structures:

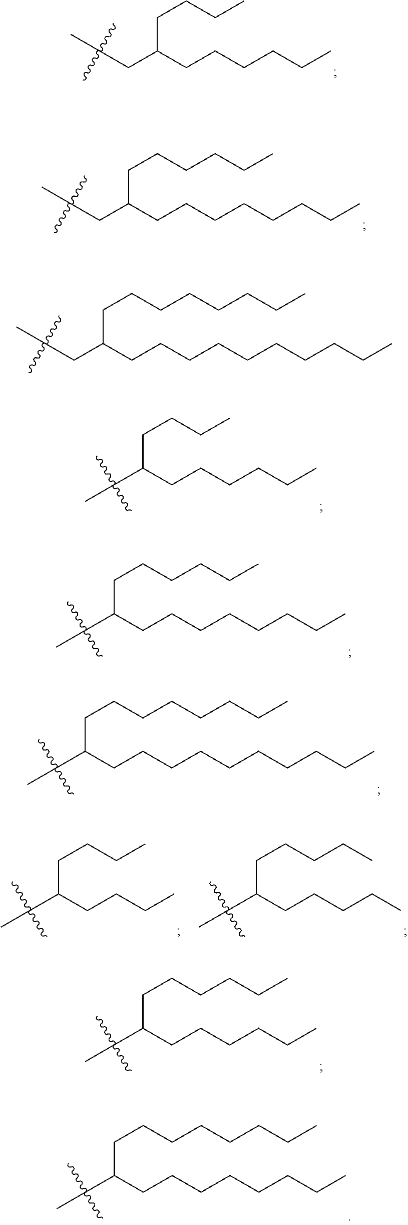

In a further embodiment, the cationic lipid is selected from any one of Compound Nos. I-1 to I-19 as specifically exemplified in Table 1, or Compound Nos. II-1 to II-20 as specifically exemplified in Table 2, of PCT Publication No. WO2018/200943, which Tables and compounds listed therein are hereby incorporated herein by reference for all purposes.

In yet another aspect, the cationic lipid is described in PCT Publication No. WO2018/078053, the contents of which is hereby incorporated herein by reference in its entirety for all purposes. In one embodiment therein, a cationic lipid of the disclosure is a compound having Formula (I):

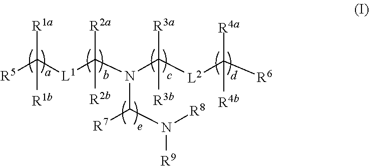

or a pharmaceutically acceptable salt, tautomer or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂, alkyl, or (b) R^{1a} is H or C₁-C₁₂, alkyl, and R^{1b} together with the carbon atom to which it is bound is taken together with an adjacent R^{1b} and the carbon atom to which it is bound to form a carbon-carbon double bond:
R^{2a} and R^{2b} are, at each occurrence, independently either (a) H or C₁-C₁₂, alkyl, or (b) R^{2a} is H or C₁-C₁₂, alkyl, and R^{2b} together with the carbon atom to which it is bound is taken together with an adjacent R^{2b} and the carbon atom to which it is bound to form a carbon-carbon double bond;
R^{1a} and R^{3b} are, at each occurrence, independently either (a) H or C₁-C₁₂, alkyl, or (b) R^{1a} is H or C₁-C₁₂, alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂, alkyl, or (b) R^{4a} is H or C₁-C₁₂, alkyl, and Rob together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂, alkyl:
R⁸ and R⁹ are each independently C₁-C₁₂, alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2.

In another embodiment, a cationic lipid of the disclosure is a compound having Formula (II):

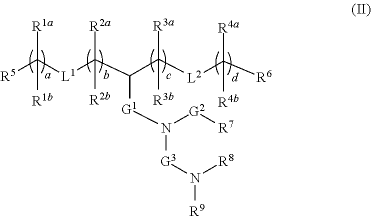

or a pharmaceutically acceptable salt, tautomer or stereoisomer thereof, wherein:
L¹ and L² are each independently —O(C═O)—, —(C═O)O—, —C(═O)—, —O—, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O—, or a direct bond;
G¹ is C₁-C₂ alkylene, —(C═O)—, —O(C═O)—, —SC(O)—, —NR^{a}C(═O)— or a direct bond
G² is —C(═O)—, —(C═O)O—, —C(═O)S—, —C(═O)NR^{a}— or a direct bond;
G³ is C₁-C₆ alkylene;
R^{a} is, at each occurrence, independently H or C₁-C₁₂ alkyl;
R^{1a} and R^{1b} are, at each occurrence, independently either: (a) H or C₁-C₁₂ alkyl; or (b) R^{1a} is H or C₁-C₁₂, alkyl, and R^{1b} together with the carbon atom to which it is bound is taken together with an adjacent Rib and the carbon atom to which it is bound to form a carbon-carbon double bond;
R^{2a} and R^{2b} are, at each occurrence, independently either: (a) H or C₁-C₁₂, alkyl; or (b) R^{2a} is H or C₁-C₁₂, alkyl, and R^{2b} together with the carbon atom to which it is bound is taken together with an adjacent R^{2b} and the carbon atom to which it is bound to form a carbon-carbon double bond;
R^{1a} and R^{3b} are, at each occurrence, independently either: (a) H or C₁-C₁₂, alkyl; or (b) R^{1a} is H or C₁-C₁₂, alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂, alkyl; or (b) R^{4a} is H or C₁-C₁₂, alkyl, and R^{4b} together with the carbon atom to which it is bound is taken together with an adjacent R^{4b} and the carbon atom to which it is bound to form a carbon-carbon double bond:
R⁵ and R⁶ are each independently H or methyl;
R⁷ is C₄-C₂₀ alkyl;
R₈ and R⁹ are each independently C₁-C₁₂, alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In another embodiment, a cationic lipid of the disclosure is a compound having Formula (III):

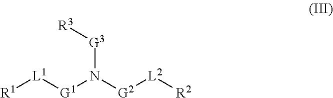

or a pharmaceutically acceptable salt, tautomer or stereoisomer thereof, wherein:
L¹ and L² are each independently —O(C═O)—, —(C═O)O—, —C(═O)—, —O—, —S(O)—, —S—S—, —C(═O)S—, —SC(═O)—, —NR^{a}C(═O)—, —C(═O)NR^{a}—, NR^{a}C(═O)NR^{a}—, —OC(═O)NR^{a}— or —NR^{a}C(═O)O—;
G¹ and G² are each independently unsubstituted C₁-C₁₂ alkylene or C₁-C₁₂ alkenylene;
G³ is C₁-C₂₄ alkylene, C₁-C₈ alkenylene, C₃-C₈ cycloalkylene, or C₃-C₈ cycloalkenylene;
R^{a} is, at each occurrence, independently H or C₁-C₁₂, alkyl;
R¹ and R² are each independently C₆-C₂₄ alkyl or C₅-C₂₄ alkenyl;
R³ is OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In a further embodiment, the cationic lipid is selected from any one of Compound Nos. I-1 to I-41 as specifically exemplified in Table 7, or Compound Nos. II-1 to II-36 as specifically exemplified in Table 8, or Compound Nos. III-1 to III-36 as specifically exemplified in Table 9, of PCT Publication No. WO2018/078053, which Tables and compounds listed therein are hereby incorporated herein by reference for all purposes.

In yet another aspect, the cationic lipid is described in PCT Publication No. WO2019/036000, the contents of which is hereby incorporated herein by reference in its entirety for all purposes. In one embodiment therein, an ionizable cationic lipid of the disclosure comprises a compound of Formula (I):

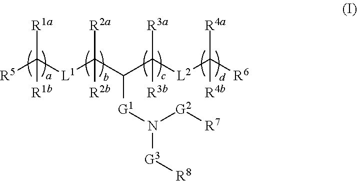

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is H or C₁-C₂₀ alkyl;
R⁸ is OH, —N(R⁹)(C═O)R¹⁰, —(CO)NR⁹R¹⁰, —NR⁹R¹⁰, —(CO)OR¹¹ or —O(C═O)R¹¹ provided that G³ is C₄-C₆ alkylene when R⁸ is —NR⁹R¹⁰,
R⁹ and R¹⁰ are each independently H or C₁-C₁₂ alkyl;
R¹¹ is aralkyl;
a, b, c and d are each independently an integer from 1 to 24; and
x is 0, 1 or 2,
wherein each alkyl, alkylene and aralkyl is optionally substituted.

In another embodiment, a cationic lipid of the disclosure is a compound having the following structures (IA) or (IB):

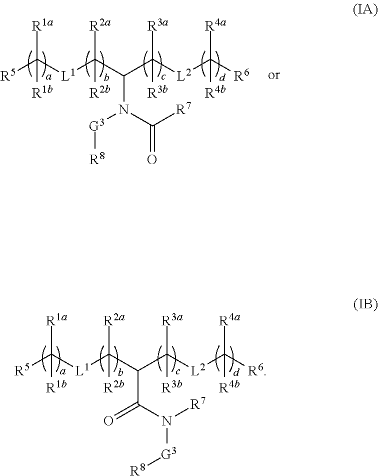

In another embodiment, a cationic lipid of the disclosure is a compound having the following structures (IC) or (ID):

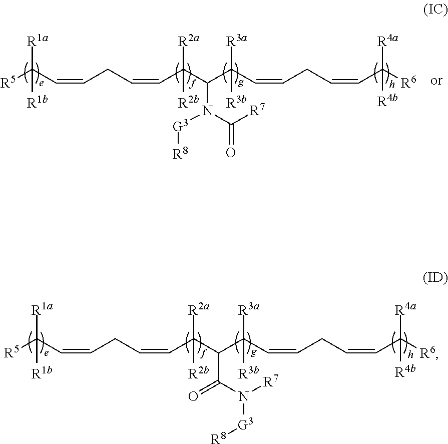

wherein e, f, g and h are each independently an integer from 1 to 12.

In a further embodiment, the cationic lipid is selected from any one of Compound Nos. 1-37 as specifically exemplified in Table 1 of PCT Publication No. WO2019/036000, which Table and compounds listed therein are hereby incorporated herein by reference for all purposes.

In yet another aspect, the cationic lipid is described in PCT Publication No. WO2019/036028, the contents of which is hereby incorporated herein by reference in its entirety for all purposes. In one embodiment therein, an ionizable cationic lipid of the disclosure comprises a compound of structure (I):

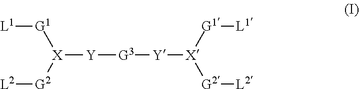

or a pharmaceutically acceptable salt or stereoisomer thereof, wherein:
X and X′ are each independently N or CR;
Y and Y′ are each independently absent, —O(C═O)—, —(C═O)O— or NR, provided that:
a) Y is absent when X is N;
b) Y′ is absent when X′ is N;
c) Y is —O(C═O)—, —(C═O)O— or NR when X is CR; and
d) Y′ is —O(C═O)—, —(C═O)O— or NR when X′ is CR,
L¹ and L^{1′} are each independently —O(C═O)R¹, —(C═O)OR¹, —C(═O)R¹, —OR¹, —S(O)_{z}R¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹, G^{1′}, G² and G^{2′} are each independently C₄-C₈ alkylene or C₂-C₈ alkenylene;
G³ is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{e} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and
wherein each alkyl, alkenyl, alkylene, alkenylene, heteroalkylene and heteroalkenylene is independently substituted or unsubstituted unless otherwise specified.

In another embodiment, a cationic lipid of the disclosure is a compound having any one of the following structures (IA), (IB), (IC), (ID), (IE), (IF), (IG) or (IH):

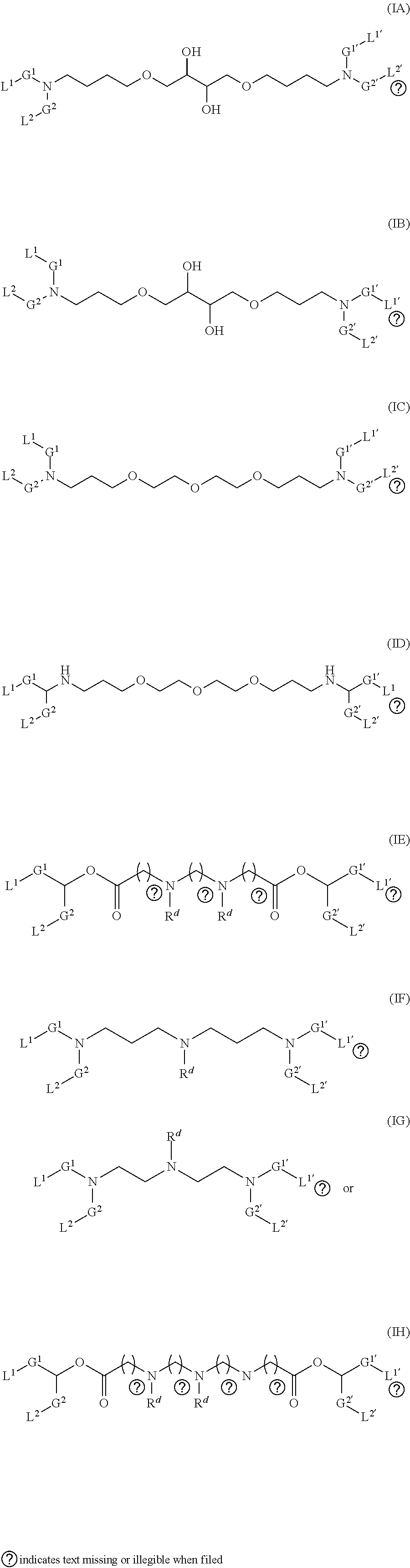

wherein R^{d} is, at each occurrence, independently H or optionally substituted C₁-C₆ alkyl.

In a further embodiment, the cationic lipid is selected from any one of Compound Nos. 1-11 as specifically exemplified in Table 1 of PCT Publication No. WO2019/036028, which Table and compounds listed therein are hereby incorporated herein by reference for all purposes.

In yet another aspect, the cationic lipid is described in PCT Publication No. WO2019/036030, the contents of which is hereby incorporated herein by reference in its entirety for all purposes. In one embodiment therein, an ionizable cationic lipid of the disclosure comprises a compound of structure (I):

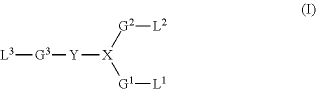

or a pharmaceutically acceptable salt, prodrug or stereoisomer thereof, wherein:
one of L¹ or L² is —O(C═O)—, —(C═O)O—, —C(═O)—, —O—, —S—S—, —C(═O)S—, —SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR S, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In another embodiment, a cationic lipid of the disclosure is a compound having any one of the following structures (IA), (IB), (IC) or (ID):

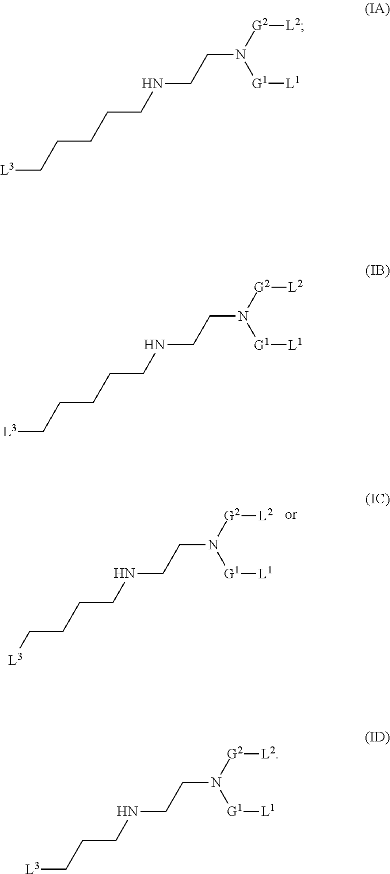

In a further embodiment, the cationic lipid is selected from any one of Compound Nos. 1-12 as specifically exemplified in Table 1 of PCT Publication No. WO2019/036030, which Table and compounds listed therein are hereby incorporated herein by reference for all purposes.

In yet another aspect, the cationic lipid is described in PCT Publication No. WO2019/036008, the contents of which is hereby incorporated herein by reference in its entirety for all purposes. In one embodiment therein, an ionizable cationic lipid of the disclosure comprises a compound of structure (I):

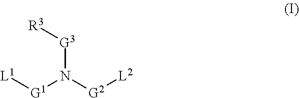

or a pharmaceutically acceptable salt or stereoisomer thereof, wherein:
L¹ is —O(C═O) R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H, C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl, wherein R⁵ is substituted with one or more substituents selected from the group consisting of —OR^{g}, —NR^{g}C(═O)R^{h}, —C(═O)NR^{g}R^{h}, —C(═O)R^{h}, —OC(═O)R^{h}, —C(═O)OR^{h} and —ORⁱOH, wherein:
R^{g} is, at each occurrence independently H or C₁-C₆ alkyl;
R^{h} is at each occurrence independently C₁-C₆ alkyl; and
Rⁱ is, at each occurrence independently C₁-C₆ alkylene; and
x is 0, 1 or 2, and
wherein each alkyl, alkenyl, alkylene, alkenylene, cycloalkylene and cycloalkenylene is independently substituted or unsubstituted unless otherwise specified.

In another embodiment, a cationic lipid of the disclosure is a compound having the following structures (IA),

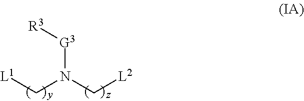

wherein y and z are each independently integers ranging from 2 to 12.

In another embodiment, a cationic lipid of the disclosure is a compound having any one of the following structures (IB), (IC), (ID) or (IE):

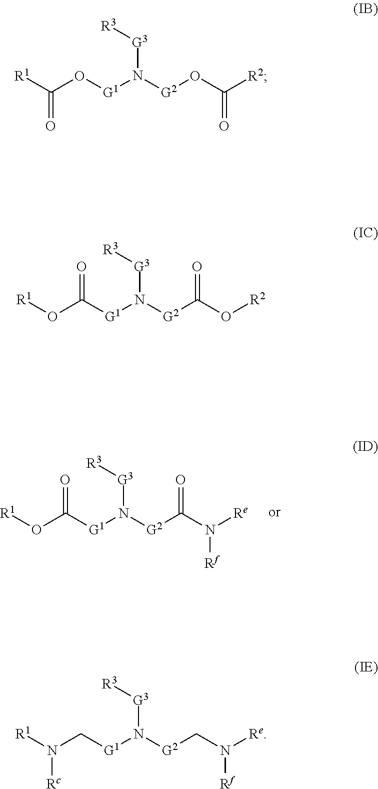

In another embodiment, a cationic lipid of the disclosure is a compound having any one of the following structures (IF), (IG), (IH) or (IJ):

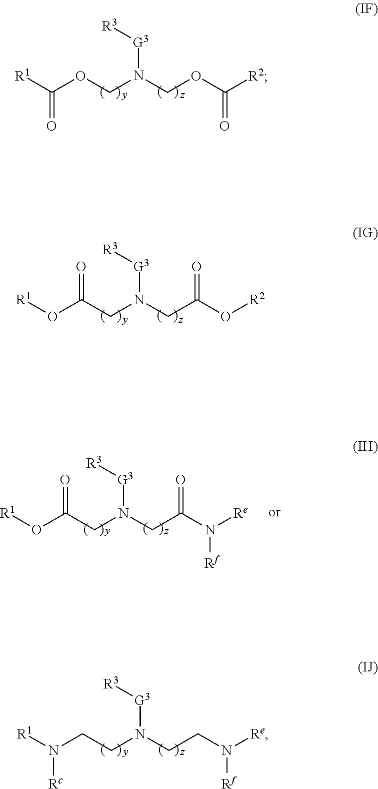

wherein y and z are each independently integers ranging from 2-12.

In a further embodiment, the cationic lipid is selected from any one of Compound Nos. 1-18 as specifically exemplified in Table 1 of PCT Publication No. WO2019/036008, which Table and compounds listed therein are hereby incorporated herein by reference for all purposes.

In yet another aspect, the cationic lipid is described in PCT Publication No. WO2020/081938, the contents of which is hereby incorporated herein by reference in its entirety for all purposes. In one embodiment therein, an ionizable cationic lipid of the disclosure comprises a compound of structure (I):

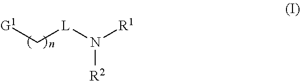

or a pharmaceutically acceptable salt, tautomer, or stereoisomer thereof, wherein:
G¹ is —N(R³)R⁴ or —OR′;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

In another embodiment, a cationic lipid of the disclosure is a compound having the following structure (IA):

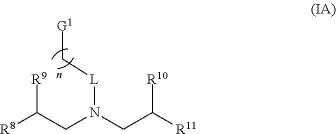

wherein
R⁸ and R⁹ are each independently H or optionally substituted branched or unbranched, saturated or unsaturated C₂-C₁₂ alkyl, provided that R⁸ and R⁹ are each independently selected such that R¹ is optionally substituted branched, saturated or unsaturated C₁₂-C₃₆ alkyl; and
R¹⁰ and R¹¹ are each independently H or optionally substituted branched or unbranched, saturated or unsaturated C₂-C₁₂ alkyl, provided that R¹⁰ and R¹¹ are each independently selected such that: R² is optionally substituted branched or unbranched, saturated or unsaturated C₁₂-C₃₆ alkyl when L is —C(═O)—; and R² is optionally substituted branched or unbranched, saturated or unsaturated C₄-C₃₆ alkyl when L is C₆-C₁₂ alkylene, C₆-C₁₂ alkenylene, or C₂-C₆ alkynylene.

In a further embodiment, the cationic lipid is selected from any one of Compound Nos. I-1 to I-22 as specifically exemplified in Table 1 of PCT Publication No. WO2020/081938, which Table and compounds listed therein are hereby incorporated herein by reference for all purposes.

In yet another aspect, the cationic lipid is described in PCT Publication No. WO2020/146805, the contents of which is hereby incorporated herein by reference in its entirety for all purposes. In one embodiment therein, an ionizable cationic lipid of the disclosure comprises a compound of structure (I):

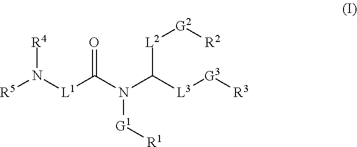

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C_{I}-C_{IX} alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

In another embodiment, a cationic lipid of the disclosure is a compound having the following structure (IA):

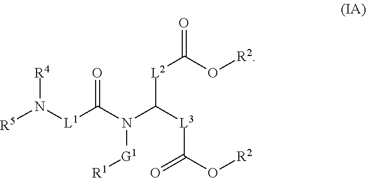

In another embodiment, a cationic lipid of the disclosure is a compound having the following structure (IB):

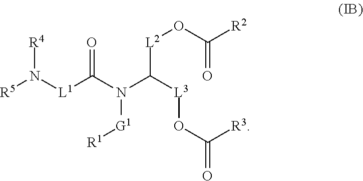

In a further embodiment, the cationic lipid is selected from any one of Compound Nos. I-1 to I-40 as specifically exemplified in Table 1 of PCT Publication No. WO2020/146805, which Table and compounds listed therein are hereby incorporated herein by reference for all purposes.

In yet another aspect, the cationic lipid is described in PCT Publication No. WO2022/016070, the contents of which is hereby incorporated herein by reference in its entirety for all purposes. In one embodiment therein, an ionizable cationic lipid of the disclosure comprises a compound of structure (I):

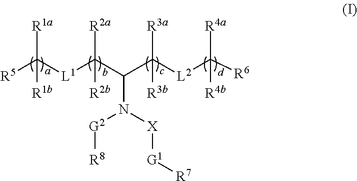

or a pharmaceutically acceptable salt, tautomer or stereoisomer thereof, wherein:
G¹ and G² are each independently C₁-C₆ alkylene;
L¹ and L² are each independently —O(C═O)— or —(C═O)O—;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is —O(C═O)R¹⁰, —(C═O)OR¹⁰, —NR⁹(C═O)R¹⁰ or —(C═O)NR⁹R¹⁰;
R⁸ is OH, —N(R¹¹)(C═O)R¹², —(C═O)NR¹¹R¹², —NR¹¹R¹², —(CO)OR′² or —O(C═O)R′²;
R⁹ is H or C₁-C₁₅ alkyl;
R¹⁰ is C₁-C₁₅ alkyl;
R¹¹ is H or C₁-C₆ alkyl;
R¹² is C₁-C₆ alkyl;
X is —(C═O)— or a direct bond; and
a, b, c and d are each independently an integer from 1 to 24;
wherein each methyl, alkyl and alkylene is independently optionally substituted.

In another embodiment, a cationic lipid of the disclosure is a compound having any one of the following structures (IA) or (IB):

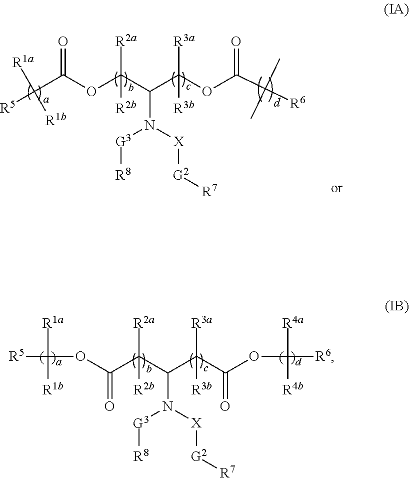

or a pharmaceutically acceptable salt, tautomer or stereoisomer thereof.

In a further embodiment, the cationic lipid is selected from any one of Compound Nos. I-1 to I-23 as specifically exemplified in Table 1 of PCT Publication No. WO2022/016070, which Table and compounds listed therein are hereby incorporated herein by reference for all purposes.

In yet another aspect, the cationic lipid is described in PCT Publication No. WO2023/114944, the contents of which is hereby incorporated herein by reference in its entirety for all purposes. In one embodiment therein, an ionizable cationic lipid of the disclosure comprises a compound of structure (I):

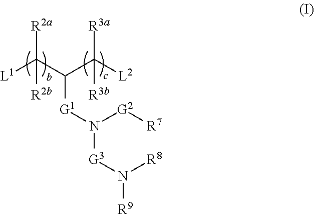

or a pharmaceutically acceptable salt, tautomer, or stereoisomer thereof, wherein: L¹ is —O(C═O)R^{1a}, —(C═O)OR^{1a}, —C(═O)R^{1a}, —OR^{1a}, —S(O)xR^{1a}, —S—SR^{1a}, —C(═O)SR^{1a}, —SC(═O)R^{1a}, —NR^{a}C(═O)R^{1a}, —C(═O)NR^{a}R^{1a}, —NR^{a}C(═O)NR^{a}R^{1a}, —OC(═O)NR^{a}R^{1a}, —NR^{a}C(═O)OR^{1a} or R^{1b}; L² is —O(C═O)R^{4a}, —(C═O)OR^{4a}, —C(═O)R^{4a}, —OR^{4a}, —S(O)ₓR^{4a}, —S—SR^{4a}, —C(═O)SR^{4a}, —SC(═O)R^{4a}, —NR^{a}C(═O)R^{4a}, —C(═O)NR^{a}R^{4a}, —NR^{a}C(═O)NR^{a}R^{4a}, —OC(═O)NR^{a}R^{4a}, —NR^{a}C(═O)OR^{4a}, or R^{4b}; G¹ is C₁-C₂ alkylene, —(C═O)—, —O(C═O)—, —SC(═O)—, —NR^{a}C(═O)— or a direct bond; G² is —C(═O)—, —(C═O)O—, —C(═O)S—, —C(═O)NR^{a}— or a direct bond; G³ is C₁-C₆ alkylene; R^{a} is H or C₁-C₁₂ alkyl; R^{1a} and R^{4a} are each independently branched C₆-C₂₄ alkyl, branched C₆-C₂₄ alkenyl, branched C₆-C₂₄ fluoroalkyl, branched C₆-C₂₄ fluoroalkenyl, C₆-C₂₄ alkylacetal or C₆-C₂₄ fluoroalkylacetal; R^{1b} and R^{4b} are each independently —CH(OR)(OR), wherein each R is independently linear or branched C₆-C₁₈ alkyl, linear or branched C₆-C₁₈ alkenyl, linear or branched C₆-C₁₈ fluoroalkyl, or linear or branched C₆-C₁₈ fluoroalkenyl R^{2a} and R^{2b} are, at each occurrence, independently H, F, C₁-C₁₂ alkyl, or C₁-C₁₂ fluoroalkyl; R^{3a} and R^{3b} are, at each occurrence, independently H, F, C₁-C₁₂ alkyl, or C₁-C₁₂ fluoroalkyl; R⁷ is H, C₄-C₂₀ alkyl, or C₂-C₁₀ fluoroalkyl; R⁸ and R⁹ are each independently C₁-C₁₂ alkyl; or R⁸ and R⁹, together with the nitrogen atom to which they are attached, form a 5, 6 or 7-membered heterocyclic ring; b and c are each independently an integer from 1 to 24; and wherein at least one of R^{2a}, R^{2b}, R^{3a}, and R^{3b} is F or C₁-C₁₂ fluoroalkyl; at least one of R^{1a} and R^{4a} is present and selected from branched C₆-C₂₄ fluoroalkyl, branched C₆-C₂₄ fluoroalkenyl and C₆-C₂₄ fluoroalkylacetal; at least one of R^{1b} and R^{4b} is present and selected from linear or branched C₆-C₁₈ fluoroalkyl and linear or branched C₆-C₁₈ fluoroalkenyl; G³ is C₁-C₆ fluoroalkylene; and/or R⁷ is C₂-C₁₀ fluoroalkyl.

In another embodiment, a cationic lipid of the disclosure is a compound having any one of the following structures (IA) or (IB):

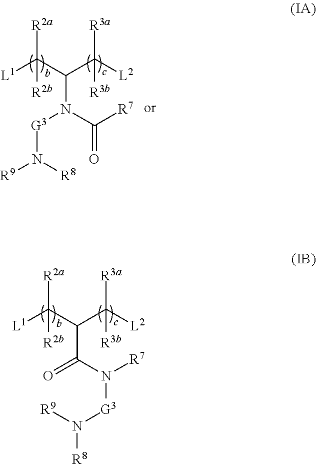

In a further embodiment, the cationic lipid is selected from any one of Compound Nos. I-1 to I-25 as specifically exemplified in Table 1 of PCT Publication No. WO2023/114944, which Table and compounds listed therein are hereby incorporated herein by reference for all purposes.

In yet another aspect, the cationic lipid is described in PCT Publication No. WO2023/114937, the contents of which is hereby incorporated herein by reference in its entirety for all purposes. In one embodiment therein, an ionizable cationic lipid of the disclosure comprises a compound of structure (I):

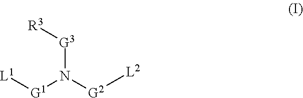

or a pharmaceutically acceptable salt or stereoisomer thereof, wherein:
L¹ is —O(C═O)R^{1a}, —(C═O)OR^{1a}, —C(═O)R^{1a}, —OR^{1a}, —S(O)ₓR^{1a}, —S—SR^{1a}, —C(═O)SR^{1a}, —SC(═O)R^{1a}, —NR^{a}C(═O)R^{1a}, —C(═O)NR^{a}R^{1a}, —NR^{a}C(═O)NR^{a}R^{1a}, —OC(═O)NR^{a}R^{1a}, NR^{a}C(═O)OR^{1a} or R^{1b};
L² is —O(C═O)R^{2a}, —(C═O)OR^{2a}, —C(═O)R^{2a}, —OR^{2a}, —S(O)ₓR^{2a}, —S—SR^{2a}, —C(═O)SR^{2a}, —SC(═O)R^{2a}, —NR^{a}C(═O)R^{2a}, —C(═O)NR^{a}R^{2a}, —NR^{a}C(═O)NR^{a}R^{2a}, —OC(═O)NR^{a}R^{2a}, —NR^{a}C(═O)OR^{2a}, or R^{2b};
G¹ and G² are each independently linear or branched C1-C12 alkylene or linear or branched C1-C12 fluoroalkylene;
G³ is linear or branched C1-C12 alkylene or linear or branched C1-C12 fluoroalkylene; each R^{a} is independently H or C1-C12 alkyl;
R^{1a} and R^{2a} are each independently branched C6-C24 alkyl, branched C6-C24 alkenyl, branched C6-C24 fluoroalkyl, branched C6-C24 fluoroalkenyl, C6-C24 alkylacetal or C6-C24 fluoroalkylacetal;
R^{1b} and R^{2b} are each independently —CH(OR)(OR), wherein each R is independently linear or branched Ce-Cis alkyl, linear or branched Ce-Cis alkenyl, linear or branched Ce-Cis fluoroalkyl, or linear or branched Ce-Cis fluoroalkenyl;
R³ is H, —OR⁵, —CN, —C(═O)OR⁴, —OC(═O)R⁴, —N(R⁵) N4, —C(═O)N(R⁴)R⁵, or —NR⁵C(═O)R⁴; and
R⁴ is H, C1-C12 alkyl, or aryl, and R⁵ is H or Ci-Ce alkyl; or R⁴ and R⁵, together with the nitrogen atom to which they are attached, form a 5, 6 or 7-membered heterocyclic ring, and
66 wherein at least one of G¹ and G² is linear or branched C1-C12 fluoroalkylene;
G³ is linear or branched C1-C12 fluoroalkylene; at least one of R^{1a} and R^{2a} is present and selected from branched C6-C24 fluoroalkyl, branched C6-C24 fluoroalkenyl and C6-C24 fluoroalkylacetal; and/or at least one of R^{1b} and R^{2b} is present and selected from linear or branched Ce-Cis fluoroalkyl and linear or branched Ce-Cis fluoroalkenyl.

In another embodiment, a cationic lipid of the disclosure is a compound having the following structure (IA):

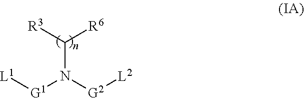

wherein:
R⁶ is, at each occurrence, independently H, F, OH or C1-C24 alkyl; and n is an integer ranging from 1 to 15.

In another embodiment, a cationic lipid of the disclosure is a compound having the following structure (IB):

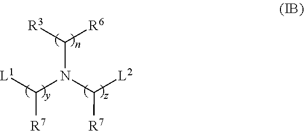

wherein: y and z are each independently integers ranging from 1 to 12; and R⁷ is, at each occurrence, independently H or F.

In another embodiment, a cationic lipid of the disclosure is a compound having any one of the following structures (IC) or (ID):

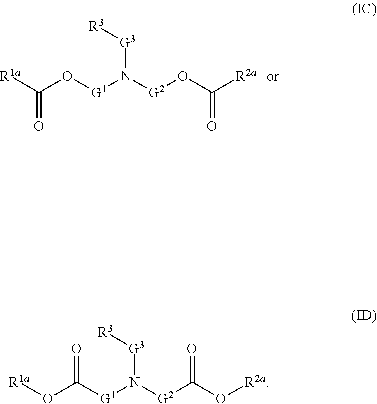

In another embodiment, a cationic lipid of the disclosure is a compound having any one of the following structures (IE) or (IF):

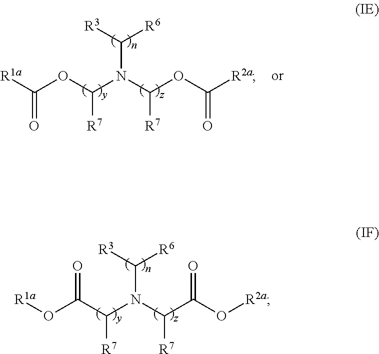

In another embodiment, a cationic lipid of the disclosure is a compound having any one of the following structures (IG) or (IH):

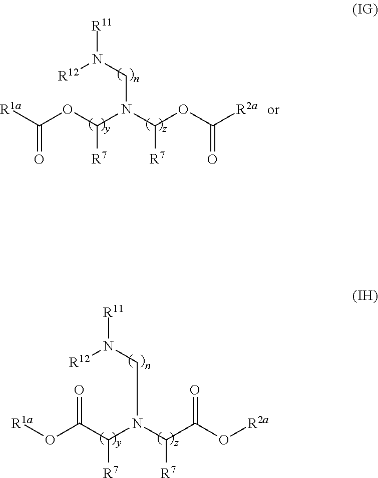

wherein R¹¹ and R¹² are each independently C1-C12 alkyl; or R¹¹ and R¹², together with the nitrogen atom to which they are attached, form a 5, 6 or 7-membered heterocyclic ring comprising one nitrogen atom.

In a further embodiment, the cationic lipid is selected from any one of Compound Nos. I-1 to I-10 as specifically exemplified in Table 1 of PCT Publication No. WO2023/114937, which Table and compounds listed therein are hereby incorporated herein by reference for all purposes.

In yet another aspect, the cationic lipid is described in PCT Publication No. WO2023/114943, the contents of which is hereby incorporated herein by reference in its entirety for all purposes. In one embodiment therein, an ionizable cationic lipid of the disclosure comprises a compound of structure (I):

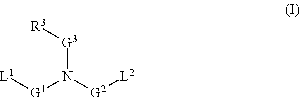

or a pharmaceutically acceptable salt, prodrug or stereoisomer thereof, wherein: L1 is —NRaC(═O)R1 or —C(═O)NRbRc; L2 is —NRdC(═O)R2 or —C(═O)NReRf; G1 and G2 are each independently C2-C12 alkylene, or C2-C12 alkenylene; G3 is C1-C24 alkylene, or C2-C24 alkenylene; Ra, Rb, Rd and Re are each independently H, C1-C16 alkyl or C2-C16 alkenyl; Rc and Rf are each independently C1-C16 alkyl or C2-C16 alkenyl; R1 and R2 are each independently C6-C24 alkyl or C6-C24 alkenyl; R3 is H, —OH, CN, —N(R4) R5; —C(═O)N(R4) R5; —N (R4) C(═O)R5; —N(R4) C(═O)OR5; —C(═O)OR6, —OC(═O)R6, —OR7, heteroaryl or aryl; R4 and R5 are each independently is H, C1-C12 alkyl, C3-C6 cycloalkyl or C3-C6 cycloalkenyl, or R4 and R5, together with the nitrogen or carbon atom to which they are bound, form a 5 to 7-membered heterocyclic ring; R6 is H, C1-C12 alkyl, C2-C12 alkenyl or aralkyl; R7 is C1-C12 alkyl optionally substituted with hydroxyl or alkoxy; and wherein each alkyl, alkenyl, alkylene, alkenylene, aryl and aralkyl is independently substituted or unsubstituted unless otherwise specified.

In another embodiment, a cationic lipid of the disclosure is a compound having the following structure (IA):

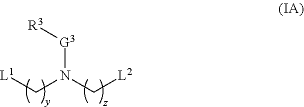

wherein y and z are each independently integers ranging from 2 to 12.

In another embodiment, a cationic lipid of the disclosure is a compound having any one of the following structures (IB) or (IC):

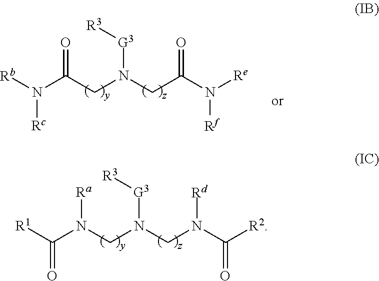

In a further embodiment, the cationic lipid is selected from any one of Compound Nos. I-1 to I-85 as specifically exemplified in Table 1 of PCT Publication No. WO2023/114943, which Table and compounds listed therein are hereby incorporated herein by reference for all purposes.

In yet another aspect, the ionizable cationic lipid is described in PCT Publication No. WO2024/054843, the contents of which is hereby incorporated herein by reference in its entirety for all purposes. In one embodiment therein, an ionizable cationic lipid of the disclosure comprises a compound of Structure (I):

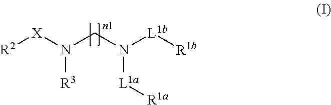

or a pharmaceutically acceptable salt, tautomer, or stereoisomer thereof, wherein:
L^{1a} and L^{1b} are each independently optionally substituted C₃-C₁₂ alkyl;
R^{1a} is —C(═O)OR^{4a} or —O(C═O)R^{4a};
R^{1b} is —C(═O)OR^{4b} or —O(C═O)R^{4b};
R² is —NR⁶ (C═O)R⁵, —(C═O)N(R⁶)R⁵ or —(C═O)OR⁷;
R³ and R⁶ are each independently hydrogen or optionally substituted C₁-C₆ alkyl;
R^{4a}, R^{4b}, and R⁵ are each independently optionally substituted alkyl;
R⁷ is optionally substituted arylalkyl;
n1 is 2, 3, 4, 5, or 6; and
X is C₂-C₆ alkylene or C₄-C₂₀ alkyleneoxide.

One embodiment provides a compound having Structure (I):

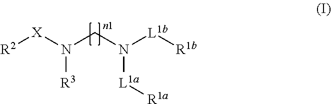

or a pharmaceutically acceptable salt, tautomer, or stereoisomer thereof, wherein:
L^{1a} and L^{1b} are each independently optionally substituted C₃-C₁₂ alkyl;
R^{1a} is —C(═O)OR^{4a} or —O(C═O)R^{4a};
R¹⁵ is —C(═O)OR^{4b} or —O(C═O)R^{4b};
R² is —NR⁶(C═O)R⁵, —(C═O)N(R⁶)R⁵ or —(C═O)OR⁷;
R³ and R⁶ are each independently hydrogen or optionally substituted C₁-C₁₂ alkyl;
R^{4a}, R^{4b}, and R⁵ are each independently optionally substituted alkyl;
R⁷ is optionally substituted C₁-C₆ alkyl or optionally substituted arylalkyl;
n1 is 2, 3, 4, 5, or 6; and
X is C₂-C₆ alkylene or C₄-C₂₀ alkyleneoxide.

In some embodiments, wherein X is:

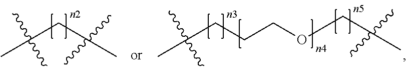

wherein:
n2 is 2, 3, 4, 5, or 6;
n3 is 0, 1, 2, 3, or 4;
n4 is 2, 3, or 4; and
n5 is 2, 3, 4, or 5.

In some embodiments, L^{1a} is C₅-C₉ alkyl. In certain embodiments, L^{1b} is C₅-C₉ alkyl. In some embodiments, L^{1a} is C₅-, C₆-, C₇-, or C₉-alkyl. In certain embodiments, L^{1b} is C₅-, C₆-, C₇-, or C₉-alkyl. In some embodiments, L^{1a} is C₅-alkyl. In certain embodiments, L^{1a} is C₉-alkyl. In some embodiments, L^{1a} is C₇-alkyl. In certain embodiments, L^{1a} is C₉-alkyl. In some embodiments. L^{1b} is C₅-alkyl. In certain embodiments. L¹⁵ is C₆-alkyl. In some embodiments. L^{1b} is C₇-alkyl. In certain embodiments, L^{1b} is C₉-alkyl. In some embodiments, L^{1a} is unsubstituted. In certain embodiments, L^{1b} is unsubstituted. In some embodiments, L^{1a} is unbranched. In certain embodiments, L^{1b} is unbranched.

In some embodiments, one of R^{1a} is —O(C═O)R^{4a}. In certain embodiments, one of R^{1a} is —(C═O)OR^{4a}. In some embodiments, R^{1b} is —O(C═O)R^{4b}. In certain embodiments, one of R^{1b} is —(C═O)OR^{4b}.

In some embodiments, R^{4a} is C₈-C₂₄-alkyl. In certain embodiments, R^{4a} is C₁₀-C₁₈-alkyl. In certain embodiments, R^{4a} is C₁₁-C₁₆-alkyl. In some embodiments. R^{4a} is C₁₁-alkyl. In certain embodiments, R^{4a} is C₁₅-alkyl. In some embodiments, R^{4a} is C₁₆-alkyl. In certain embodiments, R⁴⁰ is C₈-C₂₄-alkyl. In some embodiments, R^{4b} is C₁₀-C₁₈-alkyl. In certain embodiments, R^{4b} is C₁₁-C₁₆-alkyl. In some embodiments, R^{4b} is C₁₁-alkyl. In certain embodiments, R^{4b} is C₁₅-alkyl. In some embodiments, R^{4b} is C₁₆-alkyl.

In certain embodiments, R^{4a} is branched. In some embodiments, R^{4b} is branched. In certain embodiments, R^{4a} is unsubstituted. In some embodiments, R^{4b} is unsubstituted. In certain embodiments, R^{4a} has one of the following structures:

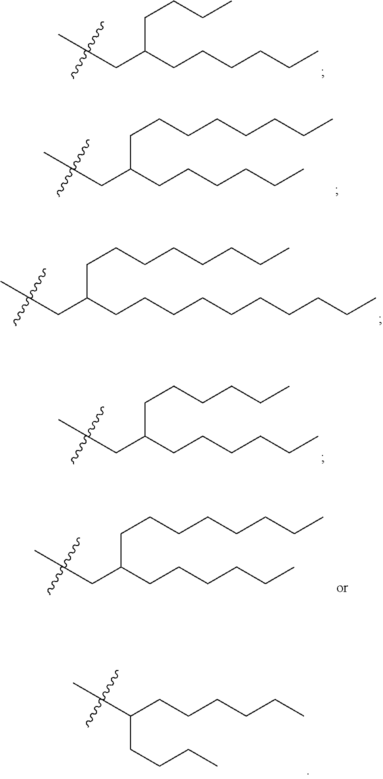 [SMILES: *CC(CCCC)CCCCCC ; *CC(CCCCCC)CCCCCCCC ; *CC(CCCCCCCC)CCCCCCCCCC ; *CC(CCCCCC)CCCCCC ; *CC(CCCCCC)CCCCCCCC ; *C(CCCC)CCCCCC]

In some embodiments, R^{4b} has one of the following structures:

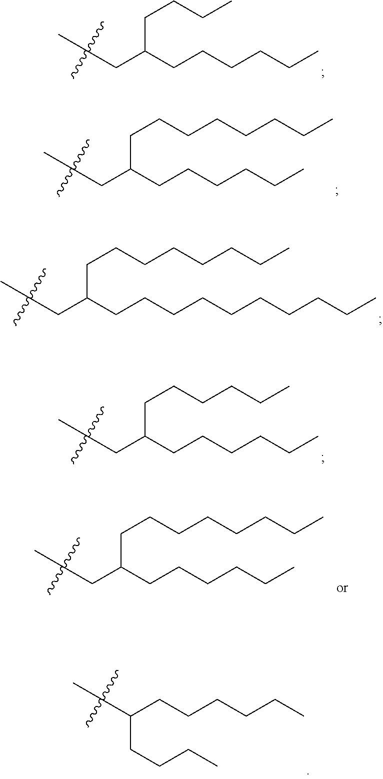 [SMILES: *CC(CCCC)CCCCCC ; *CC(CCCCCC)CCCCCCCC ; *CC(CCCCCCCC)CCCCCCCCCC ; *CC(CCCCCC)CCCCCC ; *CC(CCCCCC)CCCCCCCC ; *C(CCCC)CCCCCC]

In some embodiments, R² is —NR⁶(C═O)R⁵. In certain embodiments, R² is —(C═O)N(R)R⁵. In some embodiments. R⁵ is C₂-C₁₆-alkyl. In certain embodiments, R⁵ is C₄-C₁₃-alkyl. In some embodiments, R⁵ is C₄-, C₇-, C₈-, C₁₀-, or C₁₃-alkyl. In certain embodiments, R⁵ is unsubstituted. In some embodiments, R⁵ is substituted with hydroxyl. In some embodiments, R⁵ is branched. In certain embodiments, R⁵ is unbranched. In some embodiments, R⁵ has one of the following structures:

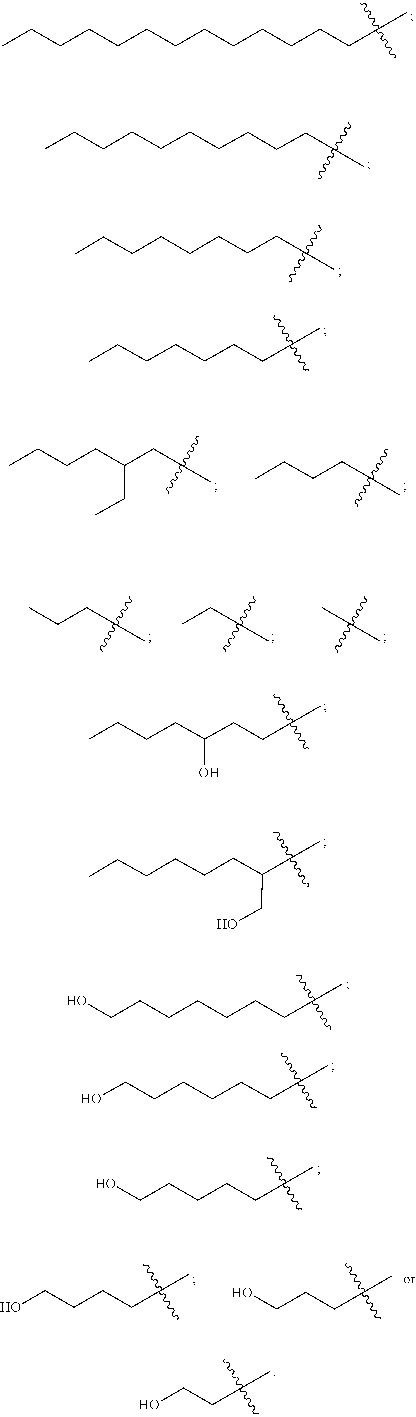

In certain embodiments, R⁵ is unbranched. In some embodiments, R⁵ has one of the following structures:

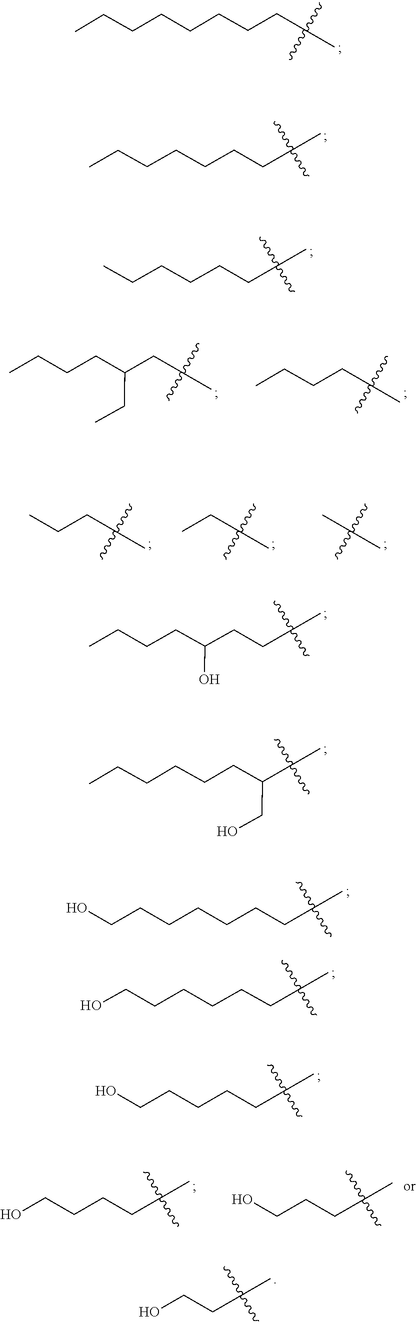

In some embodiments, R⁶ is C₁-C₆ alkyl. In some embodiments, R⁶ is C₁-C₁₀ alkyl. In certain embodiments, Re is C₁-C₄-alkyl. In some embodiments, R⁶ is C₁-, C₂-, C₃-, C₆-, C₈-, or C₁₀-alkyl. In certain embodiments, methyl, ethyl, n-butyl, n-hexyl, n-octyl, or n-decyl. In some embodiments, R⁶ is unbranched. In certain embodiments, R⁶ is methyl or n-butyl. In some embodiments, R⁶ is unsubstituted. In some embodiments, R⁶ is substituted. In some embodiments, R⁶ is C₁-C₆ alkyl substituted with one or more hydroxyl. In some embodiments. R⁶ is C₂-, C₃-, C₄-, or C₆-alkyl substituted with one or more hydroxyl. In certain embodiments, R⁶ is hydrogen. In some embodiments, R² is —(C═O)OR⁷.

In some embodiments, R⁷ is C₁-C₃ alkyl or C₇-C₁₆ arylalkyl. In certain embodiments, R⁷ is C₇-C₁₆ arylalkyl. In some embodiments, R⁷ is C₁-C₃ alkyl. In some embodiments, R⁷ is unsubstituted.

In certain embodiments, R⁷ is —CH₃ or has the following structure:

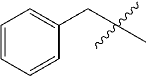 [SMILES: *Cc1ccccc1]

In certain embodiments. R⁷ has the following structure:

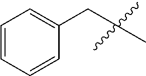 [SMILES: *Cc1ccccc1]

In some embodiments, R³ is optionally substituted C₁-C₆ alkyl. In certain embodiments. R³ is optionally substituted methyl, ethyl, n-propyl, iso-propyl, n-butyl, iso-butyl, tert-butyl, n-hexyl. In some embodiments. R³ is optionally substituted methyl. In some embodiments, R³ is C₁-C₆ alkyl substituted with one or more hydroxyl. In some embodiments, R³ is C₂- or C₄-alkyl substituted with one or more hydroxyl. In certain embodiments, R³ is unsubstituted. In some embodiments, R³ is hydrogen.

In some embodiments, X is

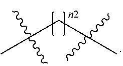

For example, in certain aspects the compound has the following Structure (II):

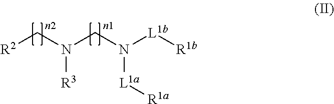

or a pharmaceutically acceptable salt, tautomer, or stereoisomer thereof. For example, in some of these embodiments n2 is 3, 4, or 5.

In other embodiments X is

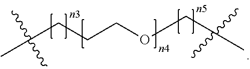

In some such embodiments, the compound has the following Structure (III):

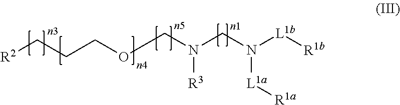

or a pharmaceutically acceptable salt, tautomer, or stereoisomer thereof. In different of these embodiments, n3 is 0 or 1. In other embodiments, n4 is 2 or 3. In some other different embodiments, n5 is 3.

In some embodiments, n1 is 3, 4, or 5. In certain embodiments, n1 is 2.

In some embodiments, the compound has one of the structures set forth in Table 24 below or a pharmaceutically acceptable salt, tautomer, or stereoisomer thereof:

TABLE 24
Representative Compounds of Structure (I)

| No. | Structure |
|---|---|
| I-1 | 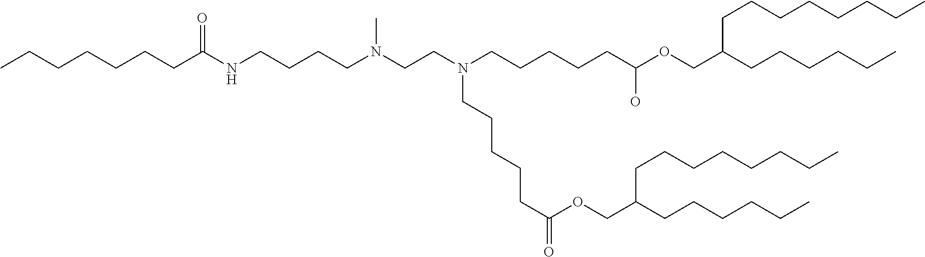 [SMILES: CCCCCCCCC(CCCCCC)COC(=O)CCCCCN(CCCCCC(O)OCC(CCCCCC)CCCCCCCC)CCN(C)CCCCNC(=O)CCCCCCC] |
| I-2 | 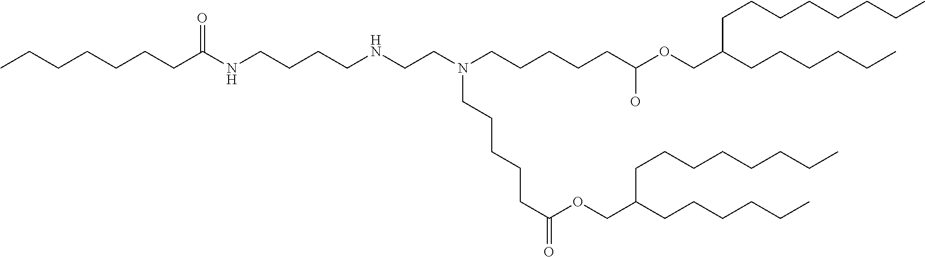 [SMILES: CCCCCCCCC(CCCCCC)COC(=O)CCCCCN(CCCCCC(O)OCC(CCCCCC)CCCCCCCC)CCNCCCCNC(=O)CCCCCCC] |
| I-3 | 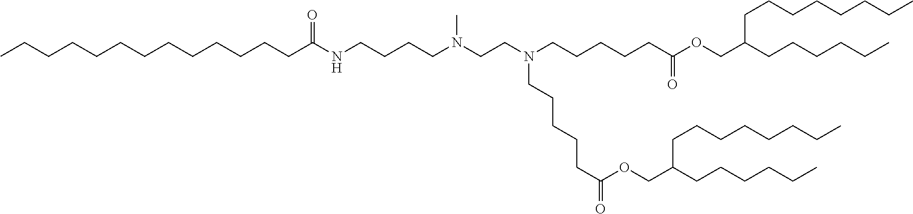 [SMILES: CCCCCCCCCCCCCC(=O)NCCCCN(C)CCN(CCCCCC(=O)OCC(CCCCCC)CCCCCCCC)CCCCCC(=O)OCC(CCCCCC)CCCCCCCC] |
| I-4 | 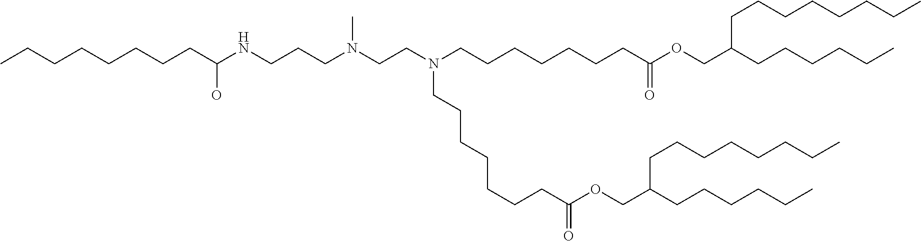 [SMILES: CCCCCCCCC(CCCCCC)COC(=O)CCCCCCCN(CCCCCCCC(=O)OCC(CCCCCC)CCCCCCCC)CCN(C)CCCNC(O)CCCCCCCC] |
| I-5 | 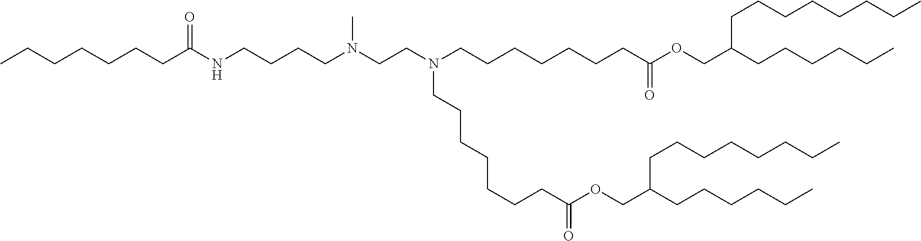 [SMILES: CCCCCCCCC(CCCCCC)COC(=O)CCCCCCCN(CCCCCCCC(=O)OCC(CCCCCC)CCCCCCCC)CCN(C)CCCCNC(=O)CCCCCCC] |
| I-6 | 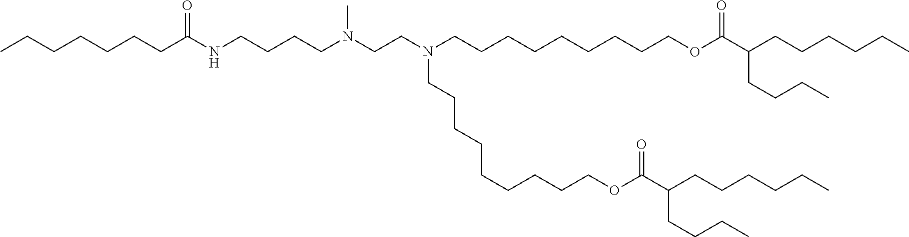 [SMILES: CCCCCCCC(=O)NCCCCN(C)CCN(CCCCCCCCCOC(=O)C(CCCC)CCCCCC)CCCCCCCCCOC(=O)C(CCCC)CCCCCC] |
| I-7 | 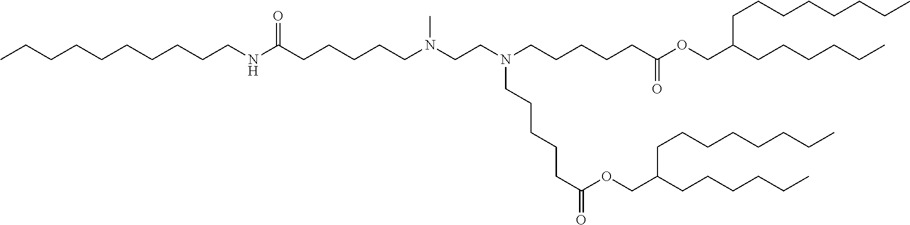 [SMILES: CCCCCCCCCCNC(=O)CCCCCN(C)CCN(CCCCCC(=O)OCC(CCCCCC)CCCCCCCC)CCCCCC(=O)OCC(CCCCCC)CCCCCCCC] |
| I-8 | 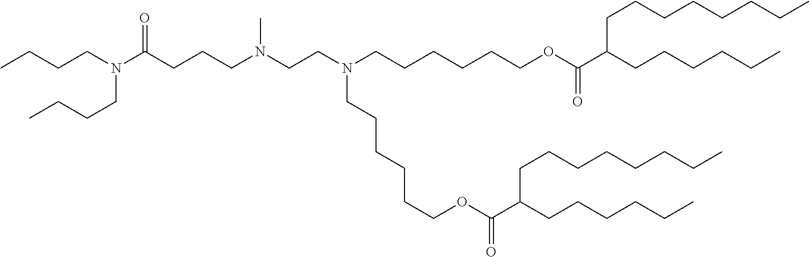 [SMILES: CCCCCCCCC(CCCCCC)C(=O)OCCCCCCN(CCCCCCOC(=O)C(CCCCCC)CCCCCCCC)CCN(C)CCCC(=O)N(CCCC)CCCC] |
| I-9 | 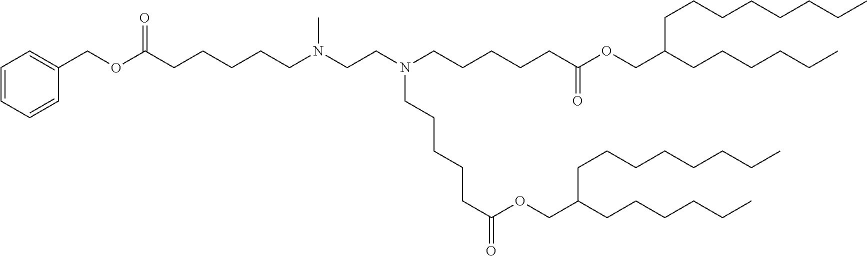 [SMILES: CCCCCCCCC(CCCCCC)COC(=O)CCCCCN(CCCCCC(=O)OCC(CCCCCC)CCCCCCCC)CCN(C)CCCCCC(=O)OCc1ccccc1] |
| I-10 | 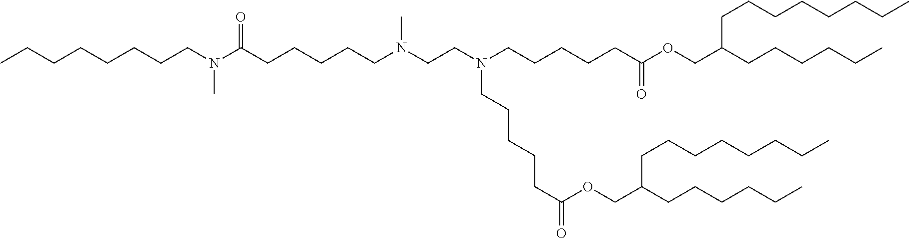 [SMILES: CCCCCCCCC(CCCCCC)COC(=O)CCCCCN(CCCCCC(=O)OCC(CCCCCC)CCCCCCCC)CCN(C)CCCCCC(=O)N(C)CCCCCCCC] |
| I-11 | 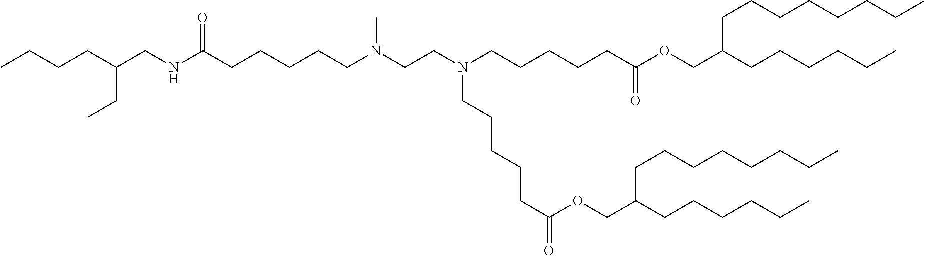 [SMILES: CCCCCCCCC(CCCCCC)COC(=O)CCCCCN(CCCCCC(=O)OCC(CCCCCC)CCCCCCCC)CCN(C)CCCCCC(=O)NCC(CC)CCCC] |
| I-12 | 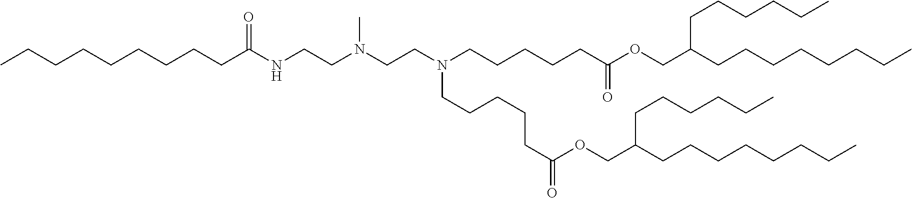 [SMILES: CCCCCCCCCC(=O)NCCN(C)CCN(CCCCCC(=O)OCC(CCCCCC)CCCCCCCC)CCCCCC(=O)OCC(CCCCCC)CCCCCCCC] |
| I-13 | 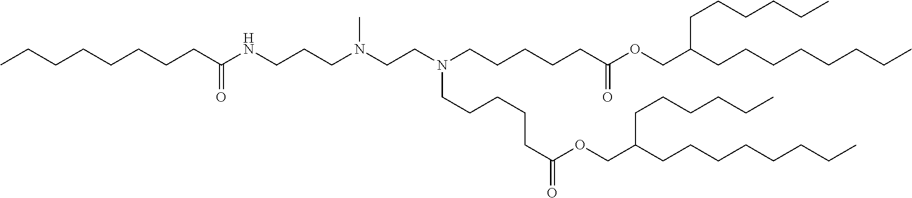 [SMILES: CCCCCCCCC(=O)NCCCN(C)CCN(CCCCCC(=O)OCC(CCCCCC)CCCCCCCC)CCCCCC(=O)OCC(CCCCCC)CCCCCCCC] |
| I-14 | 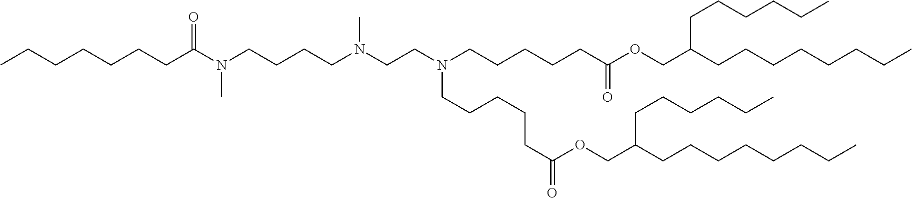 [SMILES: CCCCCCCCC(CCCCCC)COC(=O)CCCCCN(CCCCCC(=O)OCC(CCCCCC)CCCCCCCC)CCN(C)CCCCN(C)C(=O)CCCCCCC] |
| I-15 | 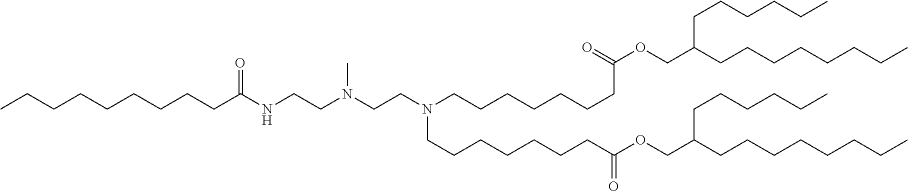 [SMILES: CCCCCCCCCC(=O)NCCN(C)CCN(CCCCCCCC(=O)OCC(CCCCCC)CCCCCCCC)CCCCCCCC(=O)OCC(CCCCCC)CCCCCCCC] |
| I-16 | 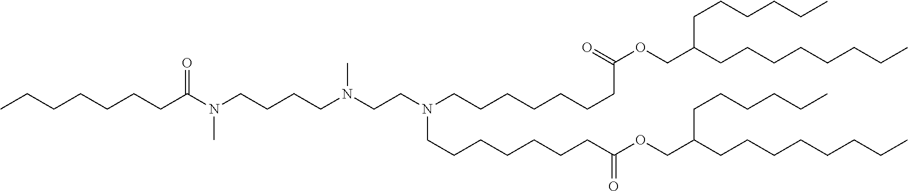 [SMILES: CCCCCCCCC(CCCCCC)COC(=O)CCCCCCCN(CCCCCCCC(=O)OCC(CCCCCC)CCCCCCCC)CCN(C)CCCCN(C)C(=O)CCCCCCC] |
| I-17 | 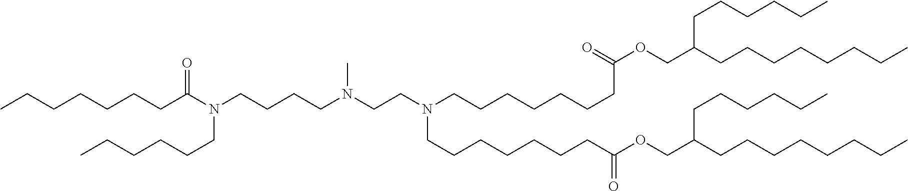 [SMILES: CCCCCCCCC(CCCCCC)COC(=O)CCCCCCCN(CCCCCCCC(=O)OCC(CCCCCC)CCCCCCCC)CCN(C)CCCCN(CCCCCC)C(=O)CCCCCCC] |
| I-18 | 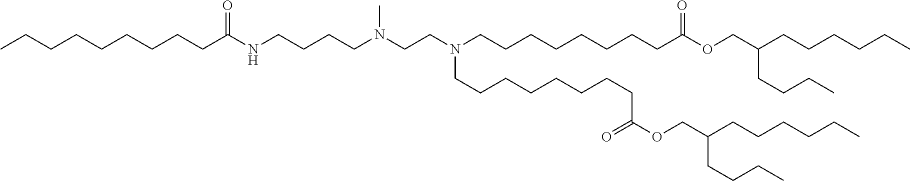 [SMILES: CCCCCCCCCC(=O)NCCCCN(C)CCN(CCCCCCCCC(=O)OCC(CCCC)CCCCCC)CCCCCCCCC(=O)OCC(CCCC)CCCCCC] |
| I-19 | 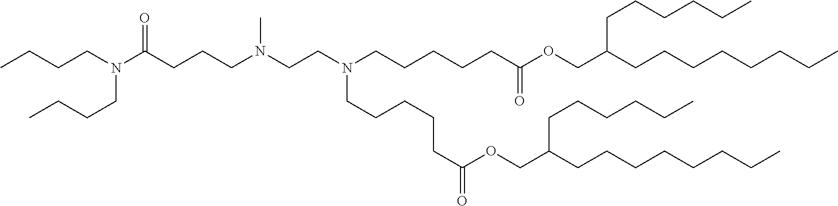 [SMILES: CCCCCCCCC(CCCCCC)COC(=O)CCCCCN(CCCCCC(=O)OCC(CCCCCC)CCCCCCCC)CCN(C)CCCC(=O)N(CCCC)CCCC] |
| I-20 | 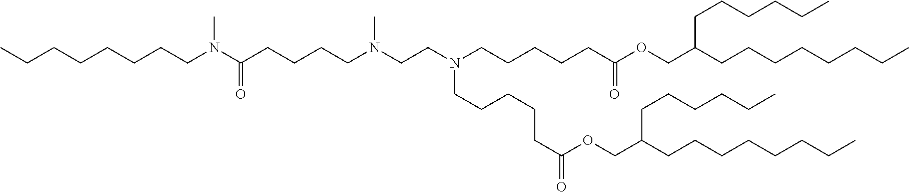 [SMILES: CCCCCCCCC(CCCCCC)COC(=O)CCCCCN(CCCCCC(=O)OCC(CCCCCC)CCCCCCCC)CCN(C)CCCCC(=O)N(C)CCCCCCCC] |
| I-21 | 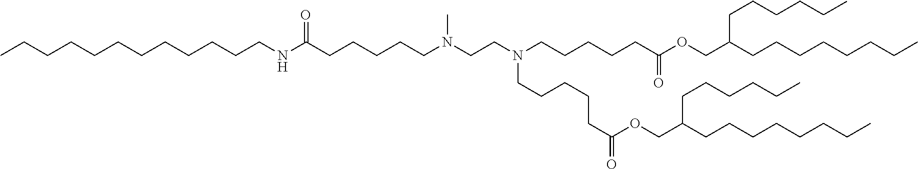 [SMILES: CCCCCCCCCCCCNC(=O)CCCCCN(C)CCN(CCCCCC(=O)OCC(CCCCCC)CCCCCCCC)CCCCCC(=O)OCC(CCCCCC)CCCCCCCC] |
| I-22 | 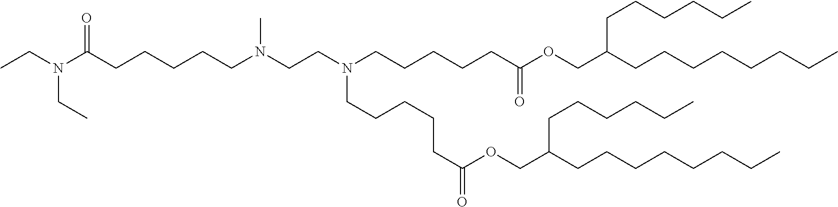 [SMILES: CCCCCCCCC(CCCCCC)COC(=O)CCCCCN(CCCCCC(=O)OCC(CCCCCC)CCCCCCCC)CCN(C)CCCCCC(=O)N(CC)CC] |
| I-23 | 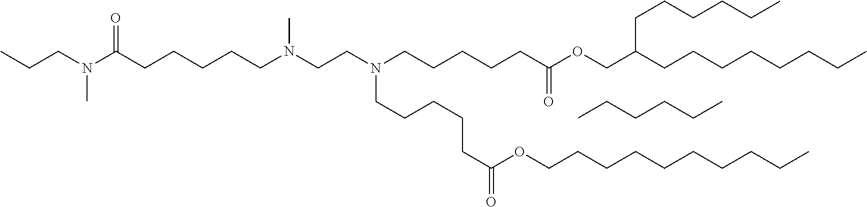 |
| I-24 | 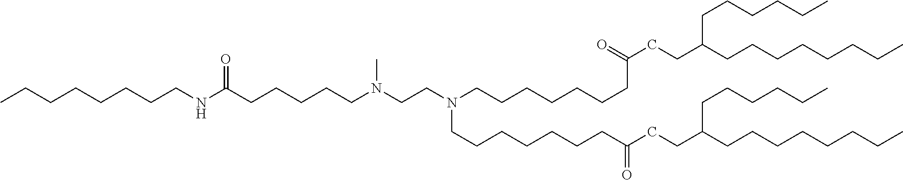 [SMILES: CCCCCCCCNC(=O)CCCCCN(C)CCN(CCCCCCCC(=O)CCC(CCCCCC)CCCCCCCC)CCCCCCCC(=O)CCC(CCCCCC)CCCCCCCC] |
| I-25 | 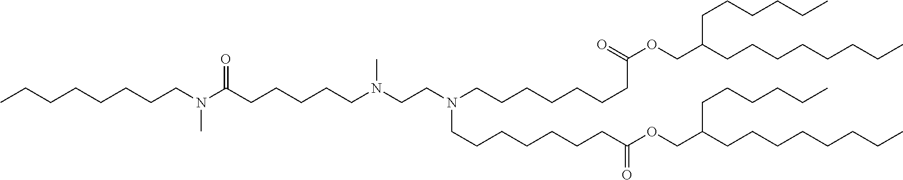 [SMILES: CCCCCCCCC(CCCCCC)COC(=O)CCCCCCCN(CCCCCCCC(=O)OCC(CCCCCC)CCCCCCCC)CCN(C)CCCCCC(=O)N(C)CCCCCCCC] |
| I-26 | 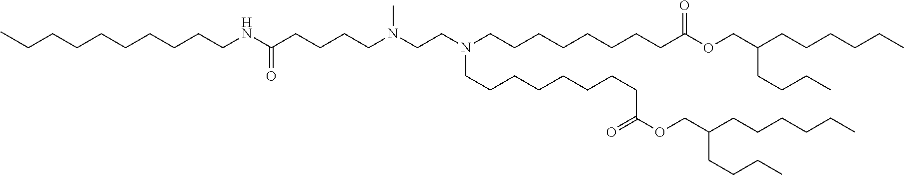 [SMILES: CCCCCCCCCCNC(=O)CCCCN(C)CCN(CCCCCCCCC(=O)OCC(CCCC)CCCCCC)CCCCCCCCC(=O)OCC(CCCC)CCCCCC] |
| I-27 | 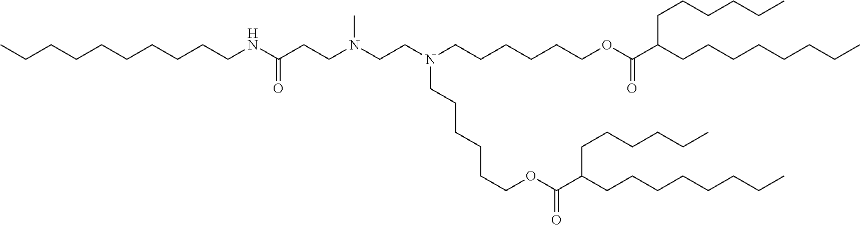 [SMILES: CCCCCCCCCCNC(=O)CCN(C)CCN(CCCCCCOC(=O)C(CCCCCC)CCCCCCCC)CCCCCCOC(=O)C(CCCCCC)CCCCCCCC] |
| I-28 | 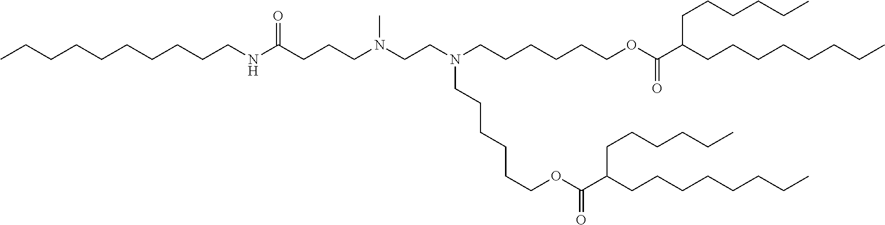 [SMILES: CCCCCCCCCCNC(=O)CCCN(C)CCN(CCCCCCOC(=O)C(CCCCCC)CCCCCCCC)CCCCCCOC(=O)C(CCCCCC)CCCCCCCC] |
| I-29 | 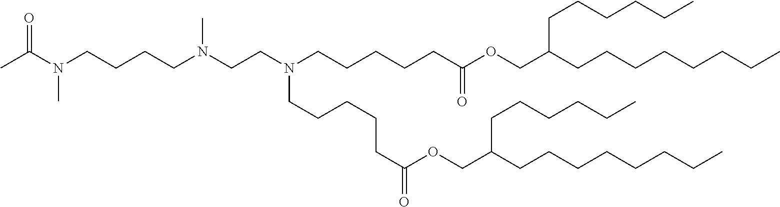 [SMILES: CCCCCCCCC(CCCCCC)COC(=O)CCCCCN(CCCCCC(=O)OCC(CCCCCC)CCCCCCCC)CCN(C)CCCCN(C)C(C)=O] |
| I-30 | 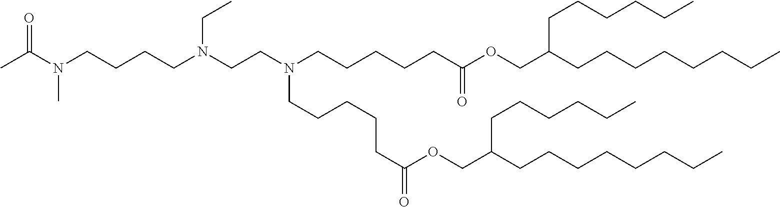 [SMILES: CCCCCCCCC(CCCCCC)COC(=O)CCCCCN(CCCCCC(=O)OCC(CCCCCC)CCCCCCCC)CCN(CC)CCCCN(C)C(C)=O] |
| I-31 | 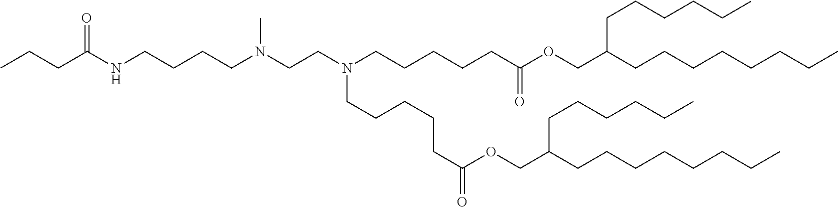 [SMILES: CCCCCCCCC(CCCCCC)COC(=O)CCCCCN(CCCCCC(=O)OCC(CCCCCC)CCCCCCCC)CCN(C)CCCCNC(=O)CCC] |
| I-32 | 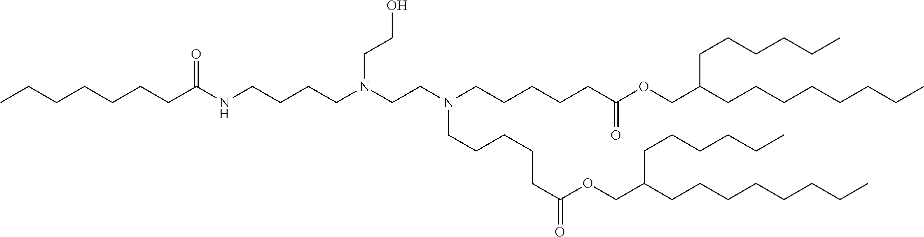 [SMILES: CCCCCCCCC(CCCCCC)COC(=O)CCCCCN(CCCCCC(=O)OCC(CCCCCC)CCCCCCCC)CCN(CCO)CCCCNC(=O)CCCCCCC] |
| I-33 | 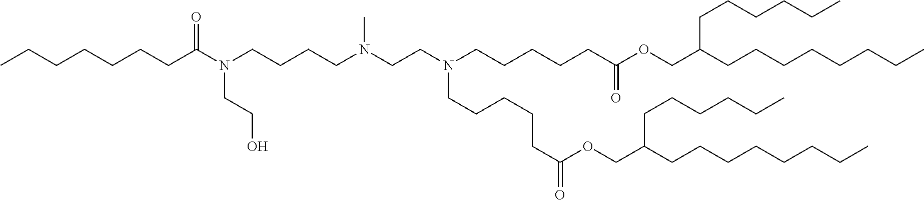 [SMILES: CCCCCCCCC(CCCCCC)COC(=O)CCCCCN(CCCCCC(=O)OCC(CCCCCC)CCCCCCCC)CCN(C)CCCCN(CCO)C(=O)CCCCCCC] |
| I-34 | 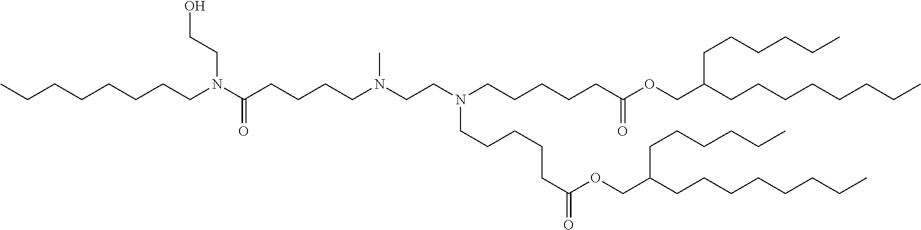 [SMILES: CCCCCCCCC(CCCCCC)COC(=O)CCCCCN(CCCCCC(=O)OCC(CCCCCC)CCCCCCCC)CCN(C)CCCCC(=O)N(CCO)CCCCCCCC] |
| I-35 | 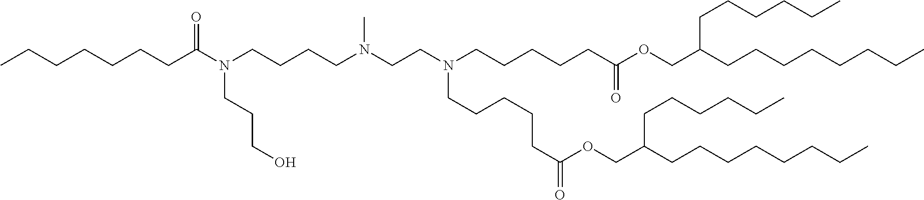 [SMILES: CCCCCCCCC(CCCCCC)COC(=O)CCCCCN(CCCCCC(=O)OCC(CCCCCC)CCCCCCCC)CCN(C)CCCCN(CCCO)C(=O)CCCCCCC] |
| I-36 | 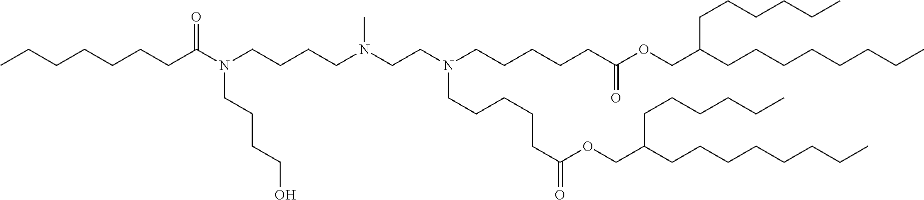 [SMILES: CCCCCCCCC(CCCCCC)COC(=O)CCCCCN(CCCCCC(=O)OCC(CCCCCC)CCCCCCCC)CCN(C)CCCCN(CCCCO)C(=O)CCCCCCC] |
| I-37 | 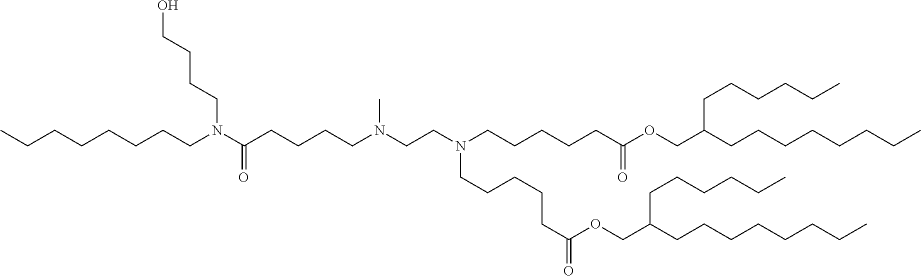 [SMILES: CCCCCCCCC(CCCCCC)COC(=O)CCCCCN(CCCCCC(=O)OCC(CCCCCC)CCCCCCCC)CCN(C)CCCCC(=O)N(CCCCO)CCCCCCCC] |
| I-38 | 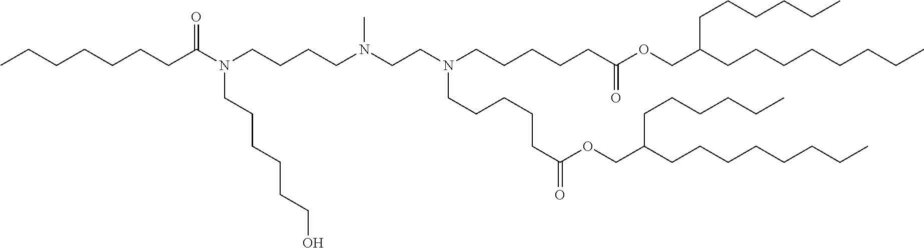 [SMILES: CCCCCCCCC(CCCCCC)COC(=O)CCCCCN(CCCCCC(=O)OCC(CCCCCC)CCCCCCCC)CCN(C)CCCCN(CCCCCCO)C(=O)CCCCCCC] |
| I-39 | 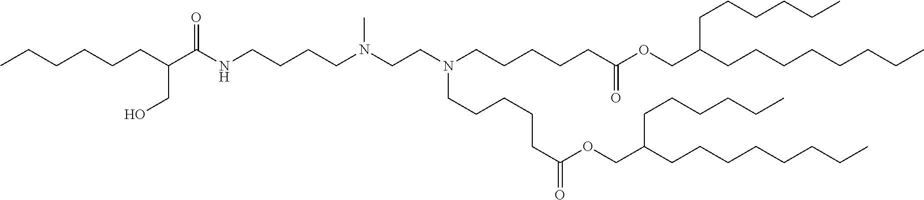 [SMILES: CCCCCCCCC(CCCCCC)COC(=O)CCCCCN(CCCCCC(=O)OCC(CCCCCC)CCCCCCCC)CCN(C)CCCCNC(=O)C(CO)CCCCCC] |
| I-40 | 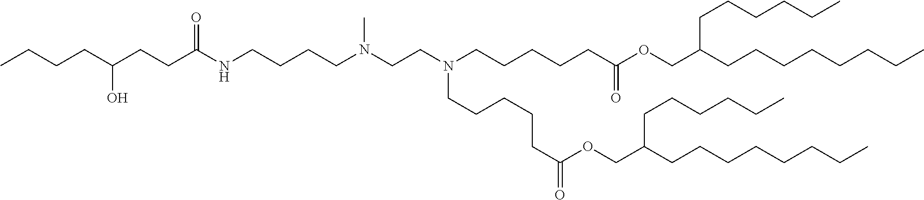 [SMILES: CCCCCCCCC(CCCCCC)COC(=O)CCCCCN(CCCCCC(=O)OCC(CCCCCC)CCCCCCCC)CCN(C)CCCCNC(=O)CCC(O)CCCC] |
| I-41 | 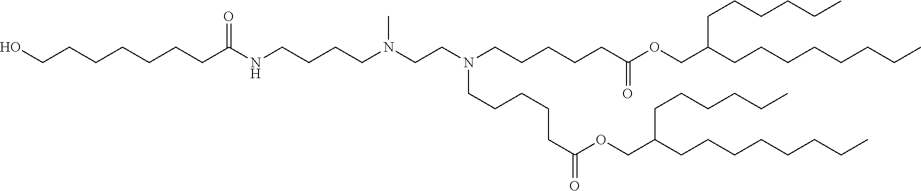 [SMILES: CCCCCCCCC(CCCCCC)COC(=O)CCCCCN(CCCCCC(=O)OCC(CCCCCC)CCCCCCCC)CCN(C)CCCCNC(=O)CCCCCCCO] |
| I-42 | 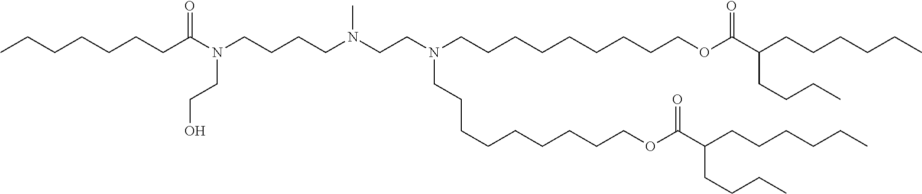 [SMILES: CCCCCCCC(=O)N(CCO)CCCCN(C)CCN(CCCCCCCCCOC(=O)C(CCCC)CCCCCC)CCCCCCCCCOC(=O)C(CCCC)CCCCCC] |
| I-43 | 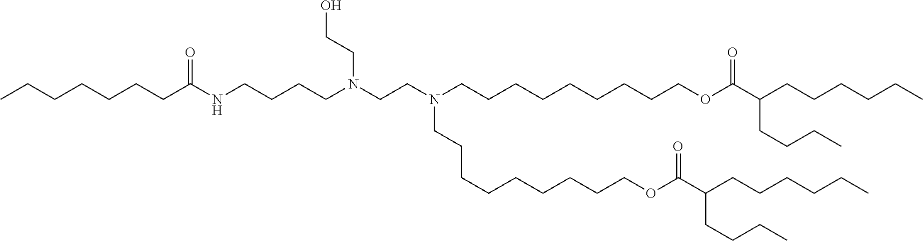 [SMILES: CCCCCCCC(=O)NCCCCN(CCO)CCN(CCCCCCCCCOC(=O)C(CCCC)CCCCCC)CCCCCCCCCOC(=O)C(CCCC)CCCCCC] |
| I-44 | 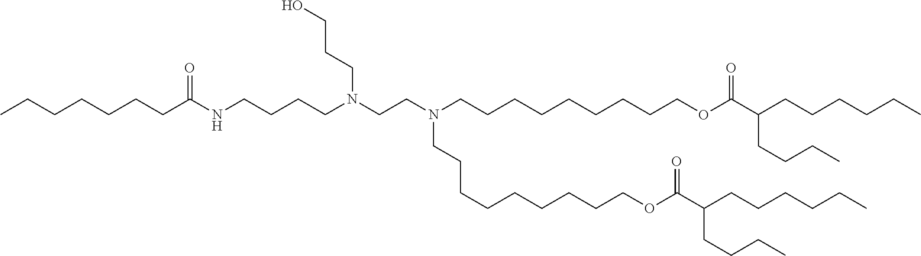 [SMILES: CCCCCCCC(=O)NCCCCN(CCCO)CCN(CCCCCCCCCOC(=O)C(CCCC)CCCCCC)CCCCCCCCCOC(=O)C(CCCC)CCCCCC] |
| I-45 | 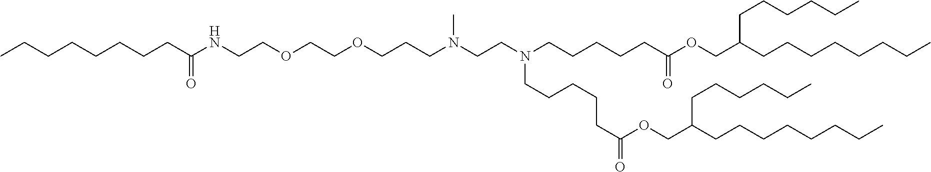 [SMILES: CCCCCCCCC(=O)NCCOCCOCCCN(C)CCN(CCCCCC(=O)OCC(CCCCCC)CCCCCCCC)CCCCCC(=O)OCC(CCCCCC)CCCCCCCC] |
| I-46 | 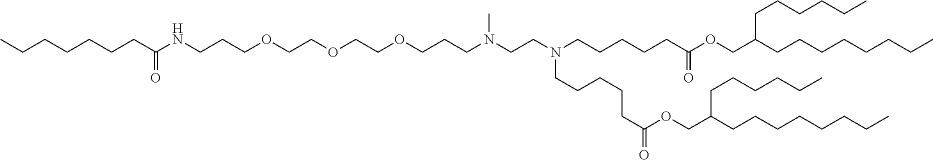 [SMILES: CCCCCCCCC(CCCCCC)COC(=O)CCCCCN(CCCCCC(=O)OCC(CCCCCC)CCCCCCCC)CCN(C)CCCOCCOCCOCCCNC(=O)CCCCCCC] |
| I-47 | 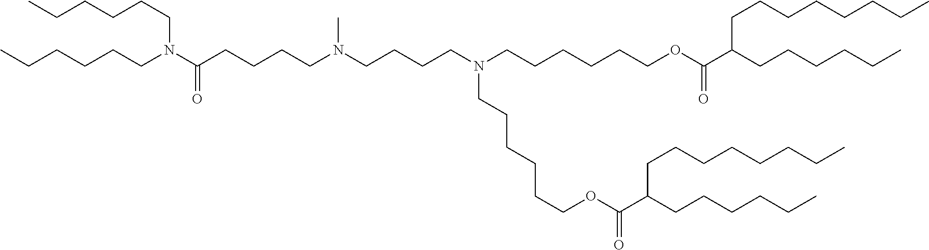 [SMILES: CCCCCCCCC(CCCCCC)C(=O)OCCCCCCN(CCCCCCOC(=O)C(CCCCCC)CCCCCCCC)CCCCN(C)CCCCC(=O)N(CCCCCC)CCCCCC] |
| I-48 | 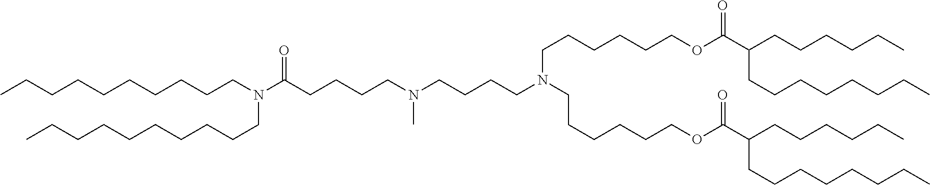 [SMILES: CCCCCCCCCCN(CCCCCCCCCC)C(=O)CCCCN(C)CCCCN(CCCCCCOC(=O)C(CCCCCC)CCCCCCCC)CCCCCCOC(=O)C(CCCCCC)CCCCCCCC] |
| I-49 | 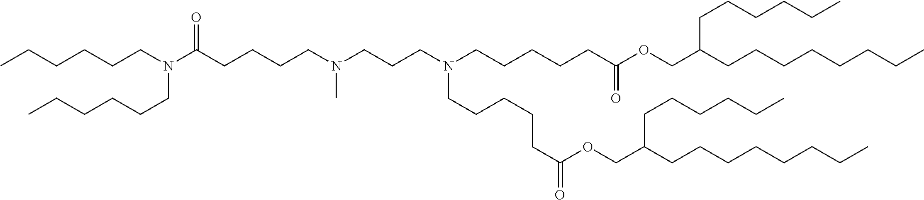 [SMILES: CCCCCCCCC(CCCCCC)COC(=O)CCCCCN(CCCCCC(=O)OCC(CCCCCC)CCCCCCCC)CCCN(C)CCCCC(=O)N(CCCCCC)CCCCCC] |
| I-50 | 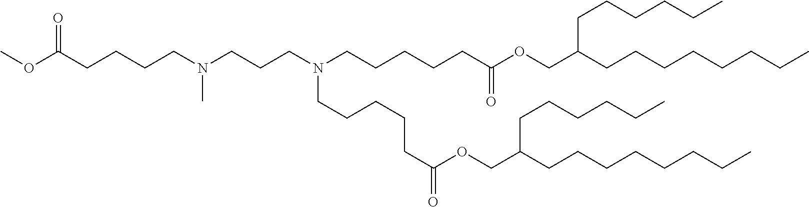 [SMILES: CCCCCCCCC(CCCCCC)COC(=O)CCCCCN(CCCCCC(=O)OCC(CCCCCC)CCCCCCCC)CCCN(C)CCCCC(=O)OC] |
| I-51 | 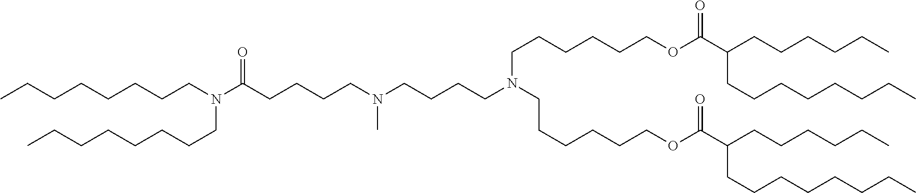 [SMILES: CCCCCCCCC(CCCCCC)C(=O)OCCCCCCN(CCCCCCOC(=O)C(CCCCCC)CCCCCCCC)CCCCN(C)CCCCC(=O)N(CCCCCCCC)CCCCCCCC] |
| I-52 | 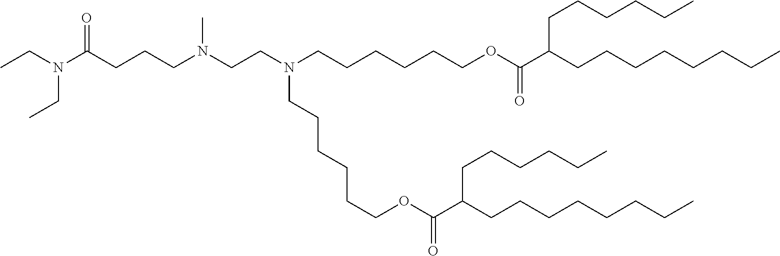 [SMILES: CCCCCCCCC(CCCCCC)C(=O)OCCCCCCN(CCCCCCOC(=O)C(CCCCCC)CCCCCCCC)CCN(C)CCCC(=O)N(CC)CC] |
| I-53 | 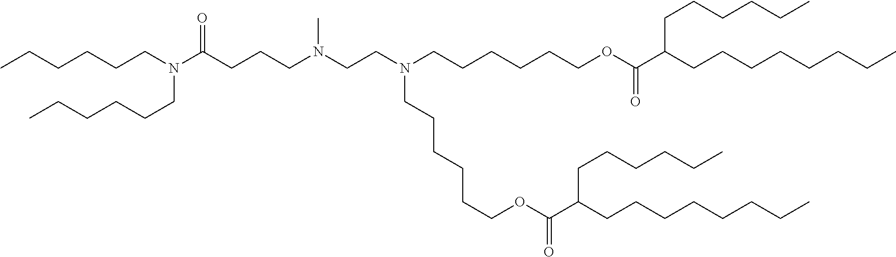 [SMILES: CCCCCCCCC(CCCCCC)C(=O)OCCCCCCN(CCCCCCOC(=O)C(CCCCCC)CCCCCCCC)CCN(C)CCCC(=O)N(CCCCCC)CCCCCC] |

The names of exemplary Compounds set forth in Table 24 above are listed in Table 25 below:

TABLE 25
Names of Exemplary Compounds set forth in Table 24

| Compound | |
|---|---|
| Number | Name |
| I-1 | bis(2-hexyldecyl) 6,6′-((2-(methyl(4- |
| | octanamidobutyl)amino)ethyl)azanediyl)dihexanoate |
| I-2 | bis(2-hexyldecyl) 6,6′-((2-((4- |
| | octanamidobutyl)amino)ethyl)azanediyl)dihexanoate |
| I-3 | bis(2-hexyldecyl) 6,6′-((2-(methyl(4- |
| | tetradecanamidobutyl)amino)ethyl)azanediyl)dihexanoate |
| I-4 | bis(2-hexyldecyl) 8,8′-((2-(methyl(3- |
| | nonanamidopropyl)amino)ethyl)azanediyl)dioctanoate |
| I-5 | bis(2-hexyldecyl) 8,8′-((2-(methyl(4- |
| | octanamidobutyl)amino)ethyl)azanediyl)dioctanoate |
| I-6 | ((2-(methyl(4-octanamidobutyl)amino)ethyl)azanediyl)bis(nonane-9,1-diyl) |
| | bis(2-butyloctanoate) |
| I-7 | bis(2-hexyldecyl) 6,6′-((2-((6-(decylamino)-6- |
| | oxohexyl)(methyl)amino)ethyl)azanediyl)dihexanoate |
| I-8 | ((2-((4-(dibutylamino)-4-oxobutyl)(methyl)amino)ethyl)azanediyl)bis(hexane- |
| | 6,1-diyl) bis(2-hexyldec anoate) |
| I-9 | bis(2-hexyldecyl) 6,6′-((2-((6-(benzyloxy)-6- |
| | oxohexyl)(methyl)amino)ethyl)azanediyl)dihexanoate |
| I-10 | bis(2-hexyldecyl) 6,6′-((2-(methyl(6-(methyl(octyl)amino)-6- |
| | oxohexyl)amino)ethyl)azanediyl)dihexanoate |
| I-11 | bis(2-hexyldecyl) 6,6′-((2-((6-((2-ethylhexyl)amino)-6- |
| | oxohexyl)(methyl)amino)ethyl)azanediyl)dihexanoate |
| I-47 | ((4-((5-(dihexylamino)-5-oxopentyl)(methyl)amino)butyl)azanediyl)bis(hexane- |
| | 6,1-diyl) bis(2-hexyldecanoate) |
| I-48 | ((4-((5-(didecylamino)-5-oxopentyl)(methyl)amino)butyl)azanediyl)bis(hexane- |
| | 6,1-diyl) bis(2-hexyldecanoate) |
| I-49 | bis(2-hexyldecyl) 6,6′-((3-((5-(dihexylamino)-5- |
| | oxopentyl)(methyl)amino)propyl)azanediyl)dihexanoate |
| I-50 | bis(2-hexyldecyl) 6,6′-((3-((5-methoxy-5- |
| | oxopentyl)(methyl)amino)propyl)azanediyl)dihexanoate |
| I-51 | ((4-((5-(dioctylamino)-5-oxopentyl)(methyl)amino)butyl)azanediyl)bis(hexane- |
| | 6,1-diyl) bis(2-hexyldecanoate) |
| I-52 | 6-((2-((3-(a,a-diethylcarbamoyl)propyl)-n-methylamino)ethyl)(6-(1- |
| | hexylnonylcarbonyloxy)hexyl)amino)hexyl 2-hexyldecanoate |
| I-53 | 6-((2-((3-(7v,7v-dihexylcarbamoyl)propyl)-n-methylamino)ethyl)(6-(1- |
| | hexylnonylcarbonyloxy)hexyl)amino)hexyl 2-hexyldecanoate |

In some aspects, 1, 2, 3, 4, 5, or more of the foregoing cationic lipids may be excluded from the LNPs of the present disclosure.

In some aspects, the RNA-LNPs comprise a cationic lipid, a RNA molecule as described herein and one or more of neutral lipids, steroids, pegylated lipids, or combinations thereof. If more than one cationic lipid is incorporated within the LNP, such percentages apply to the combined cationic lipids. In one aspect, the cationic lipid is present in the LNP in an amount such as at least, at most, exactly, or between (inclusive or exclusive) of, or about 40, 41, 42, 43, 44, 45, 46, 47, 48, 49, 50, 51, 52, 53, 54, 55, 56, 57, 58, 59 or 60 mole percent (mol %). In some aspects, two or more cationic lipids are incorporated within the LNP. If more than one cationic lipid is incorporated within the LNP, the foregoing percentages apply to the combined cationic lipids.

In some aspects of the disclosure the LNP comprises a combination or mixture of any the lipids described above.

ii. Polymer Conjugated Lipid

In some aspects, the LNPs comprise a polymer conjugated lipid. The term “polymer conjugated lipid” refers to a molecule comprising both a lipid portion and a polymer portion. An example of a polymer conjugated lipid is a pegylated lipid (e.g., polyethylene glycol-lipid, PEG-lipid). In certain aspects, the LNP comprises an additional, stabilizing lipid that is a pegylated lipid. The term “pegylated lipid” refers to a molecule comprising both a lipid portion and a polyethylene glycol portion.

Pegylated lipids are known in the art and include, but are not limited to, PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols, 2-[(polyethylene glycol)-2000]-N,N-ditetradecylacetamide, and mixtures thereof. Representative polyethylene glycol-lipids include PEG-c-DOMG, PEG-c-DMA, PEG-DSG, PEG-DPG, and PEG-s-DMG (1-(monomethoxy-polyethyleneglycol)-2,3-dimyristoylglycerol). In one aspect, the polyethylene glycol-lipid is N-[(methoxy polyethylene glycol) 2000) carbamoyl]-1,2-dimyristyloxlpropyl-3-amine (PEG-c-DMA). In one aspect, the polyethylene glycol-lipid is PEG-2000-DMG. In one aspect, the polyethylene glycol-lipid is PEG-c-DOMG. In other aspects, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanolamine (PEG-PE), a PEG succinate diacylglycerol (PEG-S-DAG) such as 4-O-(2′,3′-di(tetradecanoyloxy)propyl-1-O—((O-methoxy(polyethoxy)ethyl)butanedioate (PEG-S-DMG), a PEGylated ceramide (PEG-cer), or a PEG dialkoxypropylcarbamate such as co-methoxy (polyethoxy)ethyl-N-(2,3di(tetradecanoxy)propyl)carbamate or 2,3-di(tetradecanoxy)propyl-N-(w-methoxy(polyethoxy)ethyl)carbamate. PEG-lipids are disclosed in, e.g., U.S. Pat. No. 9,737,619, the full disclosures of which is herein incorporated by reference in its entirety for all purposes. In some aspects, 1, 2, 3, 4, 5, or more of the foregoing pegylated lipids may be excluded from the LNPs of the present disclosure.

In some aspects, the composition comprises a pegylated lipid having the following structure:

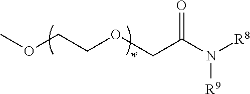

or a pharmaceutically acceptable salt, tautomer, or stereoisomer thereof, wherein:
R⁸ and R⁹ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some aspects, R⁸ and R⁹ are each independently straight, saturated alkyl chains containing from 12 to 16 carbon atoms. In some aspects, w has a mean value ranging from 43 to 53. In other aspects, the average w is or is about 45. In other different embodiments, the average w is or is about 49.

In some aspects, the lipid nanoparticles comprise a polymer conjugated lipid. In one aspect, the lipid nanoparticle comprises 2-[(polyethylene glycol)-2000]-N,N-ditetradecylacetamide (ALC-0159), having the formula:

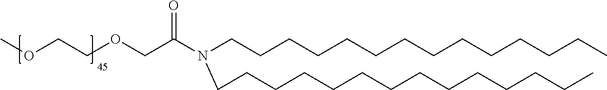

In various aspects, the molar ratio of the cationic lipid to the pegylated lipid ranges from or from about 100:1 to about 20:1, e.g., 20:1, 25:1, 30:1, 35:1, 40:1, 45:1, 50:1, 55:1, 60:1, 65:1, 70:1, 75:1, 80:1, 85:1, 90:1, 95:1, or 100:1, or any range or value derivable therein.

In certain aspects, the PEG-lipid is or is not present in the LNP in an amount from or from about 1 to about 10 mole percent (mol %) (e.g., at least, at most, exactly, or between (inclusive or exclusive) of 1, 2, 3, 4, 5, 6, 7, 8, 9, or 10 mol %), relative to the total lipid content of the nanoparticle.

In some aspects, the ratio of PEG in the lipid nanoparticle formulations may be increased or decreased and/or the carbon chain length of the PEG lipid may be modified to alter the pharmacokinetics and/or biodistribution of the lipid nanoparticle formulations.

iii. Additional Lipids

In certain aspects, the LNP comprises one or more additional lipids or lipid-like materials that stabilize particles during their formation. Suitable stabilizing or structural lipids include non-cationic lipids, e.g., neutral lipids and anionic lipids. Without being bound by any theory, optimizing the formulation of LNPs by addition of other hydrophobic moieties, such as cholesterol and lipids, in addition to an ionizable/cationic lipid or lipid-like material may enhance particle stability and efficacy of nucleic acid delivery.

As used herein, an “anionic lipid” refers to any lipid that is negatively charged at a selected pH. The term “neutral lipid” refers to any one of a number of lipid species that exist in either an uncharged or neutral zwitterionic form at physiological pH. In some aspects, additional lipids comprise one of the following neutral lipid components: (1) a phospholipid, (2) cholesterol or a derivative thereof; or (3) a mixture of a phospholipid and cholesterol or a derivative thereof.

Representative neutral lipids include phosphatidylcholines, phosphatidylethanolamines, phosphatidylglycerols, phosphatidic acids, phosphatidylserines, ceramides, sphingomyelins, dihydro-sphingomyelins, cephalins, and cerebrosides. Exemplary phospholipids include, for example, phosphatidylcholines, e.g., diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), dioleoylphosphatidylglycerol (DOPG), dipalmitoylphosphatidylglycerol (DPPG), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoyl-phosphatidylethanolamine (DOPE), 1,2-diundecanoyl-sn-glycero-phosphocholine (DUPC), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), 1-phytanoyl-phosphatidylethanolamine (DpyPE), 16-O-monomethyl PE, 16-O-dimethyl PE, 18-1-trans PE, 1-stearoyl-2-oleoylphosphatidyethanolamine (SOPE), 1,2-dielaidoyl-sn-glycero-3-phosphoethanolamine (transDOPE), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, 1, 2, 3, 4, 5, or more of the foregoing neutral lipids may be excluded from the LNPs of the present disclosure.

In one aspect, the neutral lipid is 1,2-distearoyl-sn-glycero-3-phosphocholine (DSPC), having the formula:

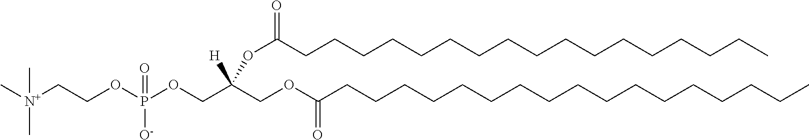 [SMILES: CCCCCCCCCCCCCCCCCC(=O)OC[C@H](COP(=O)([O-])OCC[N+](C)(C)C)OC(=O)CCCCCCCCCCCCCCCCC]

In some aspects, the LNPs comprise a neutral lipid, and the neutral lipid comprises one or more of DSPC, DPPC, DMPC, DOPC, POPC, DOPE, and/or SM. In some aspects, 1, 2, 3, 4, 5, or more of the foregoing neutral lipids may be excluded from the LNPs of the present disclosure.

In various aspects, the LNPs further comprise a steroid or steroid analogue. A “steroid” is a compound comprising the following carbon skeleton:

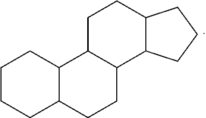 [SMILES: C1CCC2C(C1)CCC1C3CCCC3CCC21]

In certain aspects, the steroid or steroid analogue is cholesterol, fecosterol, sitosterol, ergosterol, campesterol, stigmasterol, brassicasterol, tomatidine, ursolic acid, alpha-tocopherol, and mixtures thereof. In some aspects, 1, 2, 3, 4, 5, or more of the foregoing steroid or steroid analogues may be excluded from the LNPs of the present disclosure. In certain aspects, the steroid or steroid analogue is cholesterol. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof. In some aspects, 1, 2, 3, 4, 5, or more of the foregoing cholesterol derivatives may be excluded from the LNPs of the present disclosure. In one aspect, the cholesterol has the formula:

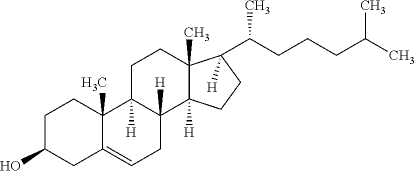

Without being bound by any theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from or from about 2:1 to about 8:1, or from or from about 10:0 to about 1:9, about 4:1 to about 1:2, or about 3:1 to about 1:1. In another aspect, the molar ratio of the cationic lipid to cholesterol ranges from or from about 2:1 to 1:1. In a further aspect, the molar ratio of the cationic lipid to the pegylated lipid ranges from or from about 100:1 to about 10:1 or from about 100:1 to about 25.1.

In some aspects, the non-cationic lipid, e.g., neutral lipid (e.g., one or more phospholipids and/or cholesterol), may comprise from or from about 0 mol % to about 90 mol %, from or from about 0 mol % to about 80 mol %, from or from about 0 mol % to about 70 mol %, from or from about 0 mol % to about 60 mol %, or from or from about 0 mol % to about 50 mol %, of the total lipid present in the particle. In some aspects, the non-cationic lipid, e.g., neutral lipid (e.g., one or more phospholipids and/or cholesterol), may or may not be at least, at most, exactly, or between (inclusive or exclusive) of 0 mol %, 10 mol %, 20 mol %, 30 mol %, 40 mol %, 50 mol %, 60 mol %, 70 mol %, 80 mol %, or 90 mol % of the total lipid present in the particle.

### VI. Characterization and Analysis of RNA Molecule

The RNA molecule described herein may be analyzed and characterized using various methods. Analysis may be performed before and/or after capping. Alternatively, analysis may be performed before and/or after poly-A capture-based affinity purification. In another aspect, analysis may be performed before and/or after additional purification steps, e.g., anion exchange chromatography and the like. For example, RNA template quality may be determined using a Bioanalyzer chip-based electrophoresis system. In other aspects, RNA template purity is analyzed using analytical reverse phase HPLC. Capping efficiency may be analyzed using, e.g., total nuclease digestion followed by MS/MS quantitation of the dinucleotide cap species vs. uncapped GTP species. In vitro efficacy may be analyzed by, e.g., transfecting an RNA molecule into a human cell line. Protein expression of the polypeptide of interest may be quantified using methods such as ELISA and/or flow cytometry. Immunogenicity may be analyzed by, e.g., transfecting RNA molecules into cell lines that indicate innate immune stimulation, e.g., PBMCs. Cytokine induction may be analyzed using, e.g., methods such as ELISA to quantify a cytokine, e.g., Interferon-α. Biodistribution may be analyzed by, e.g., bioluminescence measurements. In some aspects, 1, 2, 3, 4, 5, or more of the foregoing analytic methods may be excluded.

In some aspects, an RNA polynucleotide disclosed herein is characterized in that, when assessed in an organism administered a composition or medical preparation comprising an RNA polynucleotide, elevated expression of a gene of interest (e.g., an antigen); increased duration of expression (e.g., prolonged expression) of a gene of interest (e.g., an antigen); elevated expression and increased duration of expression (e.g., prolonged expression) of a gene of interest (e.g., an antigen); decreased interaction with IFIT1 of an RNA polynucleotide; and/or increased translation of an RNA polynucleotide; is observed relative to an appropriate reference. In some aspects, 1, 2, 3, 4, 5, or more of the foregoing characteristics may not be observed after administration of a composition or medical preparation comprising an RNA molecule of the present disclosure.

In some aspects, a reference comprises an organism administered an otherwise similar RNA polynucleotide without a m7(3′OMeG)(5′)ppp(5′)(2′OMeAi)pG2 cap. In some aspects, a reference comprises an organism administered an otherwise similar RNA polynucleotide without a cap proximal sequence disclosed herein. In some aspects, a reference comprises an organism administered an otherwise similar RNA polynucleotide with a self-hybridizing sequence.

In some aspects, elevated expression is determined at least 24 hours, at least 48 hours, at least 72 hours, at least 96 hours, or at least 120 hours after administration of a composition or medical preparation comprising an RNA polynucleotide. In some aspects, elevated expression is determined at least 24 hours after administration of a composition or medical preparation comprising an RNA polynucleotide. In some aspects, elevated expression is determined at least 48 hours after administration of a composition or medical preparation comprising an RNA polynucleotide. In some aspects, elevated expression is determined at least 72 hours after administration of a composition or medical preparation comprising an RNA polynucleotide. In some aspects, elevated expression is determined at least 96 hours after administration of a composition or medical preparation comprising an RNA polynucleotide. In some aspects, elevated expression is determined at least 120 hours after administration of a composition or medical preparation comprising an RNA polynucleotide.

In some aspects, elevated expression is determined at or at about 24-120 hours after administration of a composition or medical preparation comprising an RNA polynucleotide. In some aspects, elevated expression is determined at or at about 24-110 hours, 24-100 hours, 24-90 hours, 24-80 hours, 24-70 hours, 24-60 hours, 24-50 hours, 24-40 hours, 24-30 hours, 30-120 hours, 40-120 hours, 50-120 hours, 60-120 hours, 70-120 hours, 80-120 hours, 90-120 hours, 100-120 hours, or 110-120 hours after administration of a composition or medical preparation comprising an RNA polynucleotide.

In some aspects, expression of a gene of interest (e.g., an antigen) is or is not elevated at least 2-fold to at least 10-fold. In some aspects, expression of a gene of interest (e.g., an antigen) is elevated at least 2-fold. In some aspects, expression of a gene of interest (e.g., an antigen) is elevated at least 3-fold. In some aspects, expression of a gene of interest (e.g., an antigen) is elevated at least 4-fold. In some aspects, expression of a gene of interest (e.g., an antigen) is elevated at least 6-fold. In some aspects, expression of a gene of interest (e.g., an antigen) is elevated at least 8-fold. In some aspects, expression of a gene of interest (e.g., an antigen) is elevated at least 10-fold.

In some aspects, expression of a gene of interest (e.g., an antigen) is elevated or elevated about 2-fold to about 50-fold. In some aspects, expression of a gene of interest (e.g., an antigen) is elevated or elevated about 2-fold to about 45-fold, about 2-fold to about 40-fold, about 2-fold to about 30-fold, about 2-fold to about 25-fold, about 2-fold to about 20-fold, about 2-fold to about 15-fold, about 2-fold to about 10-fold, about 2-fold to about 8-fold, about 2-fold to about 5-fold, about 5-fold to about 50-fold, about 10-fold to about 50-fold, about 15-fold to about 50-fold, about 20-fold to about 50-fold, about 25-fold to about 50-fold, about 30-fold to about 50-fold, about 40-fold to about 50-fold, or about 45-fold to about 50-fold. In some aspects, expression of a gene of interest (e.g., an antigen) is or is not elevated at least, at most, exactly, or between (inclusive or exclusive) of 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, or any range or value derivable therein.

In some aspects, elevated expression (e.g., increased duration of expression) of a gene of interest (e.g., an antigen) persists for at least, at most, exactly, or between (inclusive or exclusive) of 24 hours, 48 hours, 72 hours, 96 hours, or 120 hours after administration of a composition or a medical preparation comprising an RNA polynucleotide. In some aspects, elevated expression of a gene of interest (e.g., an antigen) persists for at least 24 hours after administration. In some aspects, elevated expression of a gene of interest (e.g., an antigen) persists for at least 48 hours after administration. In some aspects, elevated expression of a gene of interest (e.g., an antigen) persists for at least 72 hours after administration. In some aspects, elevated expression of a gene of interest (e.g., an antigen) persists for at least 96 hours after administration. In some aspects, elevated expression of a gene of interest (e.g., an antigen) persists for at least 120 hours after administration of a composition or medical preparation comprising an RNA polynucleotide.

In some aspects, elevated expression of a gene of interest (e.g., an antigen) persists for or for about 24-120 hours after administration of a composition or medical preparation comprising an RNA polynucleotide. In some aspects, elevated expression persists for or for about 24-110 hours, 24-100 hours, 24-90 hours, 24-80 hours, 24-70 hours, 24-60 hours, 24-50 hours, 24-40 hours, 24-30 hours, 30-120 hours, 40-120 hours, 50-120 hours, 60-120 hours, 70-120 hours, 80-120 hours, 90-120 hours, 100-120 hours, or 110-120 hours after administration of a composition or medical preparation comprising an RNA polynucleotide. In some aspects, elevated expression of a gene of interest (e.g., an antigen) persists or does not persist for at least, at most, exactly, or between (inclusive or exclusive) of 24 hours, 36 hours, 48 hours, 60 hours, 72 hours, 84 hours, 96 hours, 108 hours, or 120 hours, or any range or value derivable therein.

### VII. Immune Response and Assays

As discussed herein, the disclosure concerns evoking or inducing an immune response in a subject against an RSV, PIV1, PIV3 and/or hMPV protein, e.g., a wild type or variant RSV, PIV1, PIV3 and/or hMPV F protein and/or PIV1 HN and/or PIV3 HN protein. In one aspect, the immune response may protect against or treat a subject having, suspected of having, or at risk of developing an infection or related disease, particularly those related to RSV, PIV1, PIV3 and/or hMPV. One use of the immunogenic compositions of the disclosure is to prevent RSV infections by inoculating or vaccination of a subject. In some aspects, the immunogenic compositions immunize the subject against RSV, PIV1, PIV3 and/or hMPV up to 1 year (e.g., for a single RSV, PIV1, PIV3 and/or hMPV season). In some aspects, the immunogenic compositions immunize the subject against RSV, PIV1, PIV3 and/or hMPV for up to 2 years. In some aspects, the immunogenic compositions immunize the subject against RSV, PIV1, PIV3 and/or hMPV for more than 2 years. In some aspects, the immunogenic compositions immunize the subject against RSV, PIV1, PIV3 and/or hMPV for more than 3 years. In some aspects, the immunogenic compositions immunize the subject against RSV, PIV1, PIV3 and/or hMPV for more than 4 years. In some aspects, the immunogenic compositions immunize the subject against RSV, PIV1, PIV3 and/or hMPV for 5-10 years.

### A. Immunoassays

The present disclosure includes the implementation of serological assays to evaluate whether and to what extent an immune response is induced or evoked by compositions of the disclosure. There are many types of immunoassays that may be implemented. Immunoassays encompassed by the present disclosure include, but are not limited to, those described in U.S. U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. In some aspects, the immunoassays are the various types of enzyme linked immunosorbent assays (ELISAs) and radioimmunoassays (RIA) known in the art. Immunohistochemical detection using tissue sections is also particularly useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

### B. Protective Immunity

In some aspects of the disclosure, RNA molecules encoding RSV, PIV1, PIV3 and/or hMPV preF polypeptides and/or PIV1 HN and/or PIV3 HN polypeptides, RNA-LNPs and compositions thereof, confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject. In some aspects, the RNA molecules encoding RSV, PIV1, PIV3 and/or hMPV F polypeptides and/or PIV1 HN and/or PIV3 HN polypeptides, RNA-LNPs and compositions thereof of the present disclosure may be used to induce a balanced immune response against RSV, PIV1, PIV3 and/or hMPV comprising both cellular and humoral immunity, without many of the risks associated with attenuated virus vaccination. A “humoral” immune response refers to an immune response mediated by antibody molecules, including, e.g., functional secretory (IgA) or IgG molecules that block virus infection, while a “cellular” immune response is one mediated by T-lymphocytes (e.g., CD4+ helper and/or CD8+ T cells (e.g., CTLs) and/or other white blood cells.

As used herein the phrase “immune response” or its equivalent “immunological response” refers to the development of a humoral (antibody mediated), cellular (mediated by antigen-specific T cells or their secretion products) or both humoral and cellular response directed against an antigen. Such a response may be an active response or a passive response. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject from the production of antibodies in response to the presence of an of an antigen, e.g. an RSV, PIV1, PIV3 and/or hMPV F protein and/or PIV3 HN protein encoded by an RNA molecule of the present disclosure.

As used herein “passive immunity” includes, but is not limited to, administration of activated immune effectors including cellular mediators or protein mediators (e.g., monoclonal and/or polyclonal antibodies) of an immune response. A monoclonal or polyclonal antibody composition may be used in passive immunization to treat, prevent, or reduce the severity of illness caused by infection by organisms that carry the antigen recognized by the antibody. An antibody composition may include antibodies that bind to a variety of antigens that may in turn be associated with various organisms. The antibody component may be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as viruses, including but not limited to RSV, PIV1, PIV3 and/or hMPV.

Passive immunity may be imparted to a patient or subject by administering to the patient immunoglobulins (lg) and/or other immune factors obtained from a donor or other non-patient source having a known immunoreactivity. In other aspects, an immunogenic composition of the present disclosure may be administered to a subject who then acts as a source or donor for globulin, produced in response to challenge with the immunogenic composition (“hyperimmune globulin”), that contains antibodies directed against RSV, PIV1, PIV3 and/or hMPV or other organism. A subject thus treated would donate plasma from which hyperimmune globulin would then be obtained, via conventional plasma-fractionation methodology, and administered to another subject in order to impart resistance against or to treat RSV, PIV1, PIV3 and/or hMPV infection.

For purposes of this specification and the accompanying claims the terms “epitope” and “antigenic determinant” are used interchangeably to refer to a site on an antigen to which B and/or T cells respond or recognize. B-cell epitopes may be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope may be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope may be identified by in vitro assays that measure antigen-dependent proliferation, as determined by 3H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response may be determined by proliferation assays (CD4 (+) T cells) or CTL (cytotoxic T lymphocyte) assays. The relative contributions of humoral and cellular responses to the protective or therapeutic effect of an immunogenic composition may be distinguished by separately isolating IgG and T-cells from an immunized syngeneic animal and measuring protective or therapeutic effect in a second subject.

As used herein, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins. Under normal physiological conditions antibodies are found in plasma and other body fluids and in the membrane of certain cells and are produced by lymphocytes of the type denoted B cells or their functional equivalent.

As used herein the terms “immunogenic agent” or “immunogen” or “antigen” are used interchangeably to describe a molecule capable of inducing an immunological response against itself on administration to a recipient, either alone, in conjunction with an adjuvant, or presented on a display vehicle.

### VIII. Compositions

In some aspects, RNA molecules and/or RNA-LNPs disclosed herein may be administered in a pharmaceutical composition or a medicament and may be administered in the form of any suitable pharmaceutical composition. In some aspects, a pharmaceutical composition is for therapeutic and/or prophylactic treatment. In one aspect, the disclosure relates to a composition for administration to a host. In some aspects, the host is a human. In other aspects, the host is a non-human.

Formulations of the compositions described herein may be prepared by any method known or hereafter developed in the art of pharmacology. In general, such preparatory methods include the step of bringing an active ingredient (e.g., RNA molecules and/or RNA-LNPs) into association with an excipient and/or one or more other accessory ingredients, and then, if necessary and/or desirable, dividing, shaping and/or packaging the product into a desired single- or multi-dose unit. A pharmaceutical composition or formulation in accordance with the present disclosure can be prepared, packaged, and/or sold in bulk, as a single unit dose, and/or as a plurality of single unit doses. Relative amounts of the active ingredient, the pharmaceutically acceptable excipient, and/or any additional ingredients in a composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w), or at least, at most, exactly, or between (inclusive or exclusive) any two of 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 5%, 10%, 15%, 20%, 25%, 30%, 35%, 40%, 45%, 50%, 55%, 60%, 65%, 70%, 75%, 80%, 85%, 90%, 95%, or 100% (w/w) active ingredient. General considerations in the formulation and/or manufacture of pharmaceutical agents, such as the compositions described herein, may be found, for example, in Remington: The Science and Practice of Pharmacy 21st ed., Lippincott Williams & Wilkins, 2005 (incorporated herein by reference in its entirety).

In some aspects, RNA molecules and/or RNA-LNPs disclosed herein may be administered in a pharmaceutical composition which may be formulated into preparations in solid, semi-solid, liquid, lyophilized, frozen, and/or gaseous forms. In some aspects, an RNA molecule and/or RNA-LNPs disclosed herein may be administered in a pharmaceutical composition which may comprise a pharmaceutically acceptable carrier and may optionally comprise one or more adjuvants, stabilizers, salts, buffers, preservatives, and optionally other therapeutic agents. In some aspects, a pharmaceutical composition disclosed herein comprises one or more pharmaceutically acceptable carriers, diluents and/or excipients. In some aspects, pharmaceutical compositions do not include an adjuvant (e.g., they are adjuvant free).

The term “excipient” as used herein refers to a substance which may be present in a pharmaceutical composition of the present disclosure but is not an active ingredient. Examples of excipients, include without limitation, carriers, diluents (e.g., solvents, dispersion media, and/or other liquid vehicles, dispersion or suspension aids), granulating and/or dispersing agents, surface active agents, isotonic agents, thickening and/or emulsifying agents, preservatives, binders, lubricants and/or oil, coloring, sweetening and/or flavoring agents, stabilizers, antioxidants, antimicrobial and/or antifungal agents, osmolality adjusting agents, pH adjusting agents, buffers, chelants, cryoprotectants, and/or bulking agents. In some aspects, 1, 2, 3, 4, 5, or more of the foregoing excipients may be excluded from the pharmaceutical compositions disclosed herein.

The term “carrier” refers to a component which may be natural, synthetic, organic, or inorganic, in which the active component is combined in order to facilitate, enhance and/or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer's solution, Ringer's lactate solution, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some aspects, the pharmaceutical composition of the present disclosure includes sodium chloride. In some aspects, 1, 2, 3, 4, 5, or more of the foregoing carriers may be excluded from the pharmaceutical compositions disclosed herein.

The term “diluent” relates a diluting and/or thinning agent. Moreover, the term “diluent” includes any one or more of fluid, liquid or solid suspension and/or mixing media. Examples of suitable diluents for use in a pharmaceutical compositions of the present disclosure include, without limitation, ethanol, glycerol, saline, water, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof. In some aspects, 1, 2, 3, 4, 5, or more of the foregoing diluents may be excluded from the pharmaceutical compositions disclosed herein.

Examples of suitable granulating and/or dispersing agents include, without limitation, starches, pregelatinized starches, or microcrystalline starch, alginic acid, guar gum, agar, poly(vinyl-pyrrolidone), (providone), cross-linked poly(vinyl-pyrrolidone) (crospovidone), cellulose, methylcellulose, carboxymethyl cellulose, cross-linked sodium carboxymethyl cellulose (croscarmellose), magnesium aluminum silicate (VEEGUM®), sodium lauryl sulfate, etc., and/or combinations thereof. In some aspects, 1, 2, 3, 4, 5, or more of the foregoing granulating and/or dispersing agents may be excluded from the pharmaceutical compositions disclosed herein.

Examples of suitable surface active agents for use in a pharmaceutical compositions of the present disclosure include, without limitation, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN®80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ® 30]), PLUORINC® F 68, POLOXAMER® 188, etc. and/or combinations thereof. In some aspects, 1, 2, 3, 4, 5, or more of the foregoing surface active agents may be excluded from the pharmaceutical compositions disclosed herein.

Examples of suitable preservatives for use in a pharmaceutical compositions of the present disclosure include, without limitation, benzalkonium chloride, chlorobutanol, paraben, thimerosal, vitamin A, vitamin C, vitamin E, beta-carotene, citric acid, ascorbic acid, butylated hydroxyanisole, ethylenediamine, sodium lauryl sulfate (SLS), sodium lauryl ether sulfate (SLES), etc., and combinations thereof. In some aspects, 1, 2, 3, 4, 5, or more of the foregoing preservatives may be excluded from the pharmaceutical compositions disclosed herein.

Examples of suitable antimicrobial and/or antifungal agents for use in a pharmaceutical compositions of the present disclosure include, without limitation, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof. In some aspects, 1, 2, 3, 4, 5, or more of the foregoing antimicrobial and/or antifungal agents may be excluded from the pharmaceutical compositions disclosed herein.

Examples of suitable binders for use in a pharmaceutical compositions of the present disclosure include, without limitation, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof. In some aspects, 1, 2, 3, 4, 5, or more of the foregoing binders may be excluded from the pharmaceutical compositions disclosed herein.

Examples of suitable lubricants and/or oil for use in a pharmaceutical compositions of the present disclosure include, without limitation, magnesium stearate, calcium stearate, stearic acid, silica, talc, malt, hydrogenated vegetable oils, polyethylene glycol, sodium benzoate, sodium or magnesium lauryl sulfate, etc., and combinations thereof. In some aspects, 1, 2, 3, 4, 5, or more of the foregoing lubricants and/or oils may be excluded from the pharmaceutical compositions disclosed herein.

Examples of suitable antioxidants for use in a pharmaceutical compositions of the present disclosure include, without limitation, alpha tocopherol, ascorbic acid, ascorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof. In some aspects, 1, 2, 3, 4, 5, or more of the foregoing antioxidants may be excluded from the pharmaceutical compositions disclosed herein.

Examples of suitable osmolality adjusting agents, pH adjusting agents, and buffers for use in a pharmaceutical compositions of the present disclosure include, without limitation, sodium phosphate, sodium citrate, sodium succinate, histidine (or histidine-HCl), sodium malate, sodium carbonate, etc., and/or combinations thereof. In some aspects, 1, 2, 3, 4, 5, or more of the foregoing osmolality adjusting agents may be excluded from the pharmaceutical compositions disclosed herein.

Examples of suitable chelating agents for use in a pharmaceutical compositions of the present disclosure include, without limitation, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof. In some aspects, 1, 2, 3, 4, 5, or more of the foregoing chelating agents may be excluded from the pharmaceutical compositions disclosed herein.

Examples of suitable cryoprotectants for use in a pharmaceutical compositions of the present disclosure include, without limitation, mannitol, sucrose, trehalose, lactose, glycerol, dextrose, etc., and combinations thereof. In some aspects, 1, 2, 3, 4, 5, or more of the foregoing cryoprotectants may be excluded from the pharmaceutical compositions disclosed herein.

Examples of suitable bulking agents include, without limitation, sucrose, trehalose, mannitol, glycine, lactose, raffinose, and combinations thereof. In some aspects, 1, 2, 3, 4, 5, or more of the foregoing bulking agents may be excluded from the pharmaceutical compositions disclosed herein.

Compositions can be formulated using one or more excipients (e.g., one or more carriers and/or diluents) to, e.g.: (1) increase stability; (2) increase cell transfection; (3) permit the sustained and/or delayed release (e.g., from a depot formulation); (4) alter the biodistribution (e.g., target to specific tissues and/or cell types); (5) increase the translation of encoded protein in vivo; and/or (6) alter the release profile of encoded protein (antigen) in vivo. In some aspects, 1, 2, 3, 4, 5, or more of the foregoing excipient purposes may be excluded. Pharmaceutically acceptable excipients (e.g., carriers and/or diluents) for therapeutic use are well known in the pharmaceutical art, and are described, for example, in Remington's Pharmaceutical Sciences, Mack Publishing Co. (A. R Gennaro edit. 1985).

Pharmaceutical excipients (e.g., carriers and/or diluents) may be selected with regard to the intended route of administration and standard pharmaceutical practice.

In some aspects, the composition comprises an RNA molecule comprising an open reading frame encoding an immunogenic polypeptide. In some aspects, the immunogenic polypeptide comprises a RSV, PIV1, PIV3 or hMPV antigen. In some aspects, the immunogenic polypeptide comprises a RSV, PIV1, PIV3 or hMPV F protein or a fragment or variant thereof.

In some aspects, the composition comprises an RNA molecule comprising an open reading frame encoding a full-length RSV, PIV1, PIV3 or hMPV F protein. In some aspects, the encoded immunogenic polypeptide is a truncated RSV, PIV1, PIV3 or hMPV F protein. In some aspects, the encoded immunogenic polypeptide is a variant of a RSV, PIV1, PIV3 or hMPV F protein. In some aspects, the encoded immunogenic polypeptide is a fragment of a RSV, PIV1, PIV3 or hMPV F protein.

### A. Immunogenic Compositions Including LNPs

In some aspects, a pharmaceutical composition comprises an RNA molecule (e.g., polynucleotide) disclosed herein formulated with a lipid-based delivery system. Thus, some aspects, the composition includes a lipid-based delivery system (e.g., LNPs) (e.g., a lipid-based vaccine), which delivers a nucleic acid molecule to the interior of a cell, where it may then replicate, inhibit protein expression of interest, and/or express the encoded polypeptide of interest. The delivery system may have adjuvant effects which enhance the immunogenicity of an encoded antigen. In some aspects, the composition comprises at least one RNA molecule encoding a polypeptide complexed with, encapsulated in, and/or formulated with one or more lipids, and forming lipid nanoparticles (LNPs), liposomes, lipoplexes and/or nanoliposomes. In some aspects, the composition comprises a lipid nanoparticle. Thus, in certain aspects, the present disclosure concerns compositions comprising one or more lipids associated with a nucleic acid or a polypeptide/peptide (e.g., RSV, PIV1, PIV3 and/or hMPV RNA-LNPs).

The immunogenic composition including a lipid-based delivery system may further include one or more salts and/or one or more pharmaceutically acceptable surfactants, preservatives, carriers, diluents, and/or excipients, in some cases. In some aspects, the immunogenic composition including a lipid-based delivery system further includes a pharmaceutically acceptable vehicle. In some aspects, each of a buffer, stabilizing agent, and optionally a salt, may be included in the immunogenic composition including a lipid-based delivery system. In other aspects, any one or more of a buffer, stabilizing agent, salt, surfactant, preservative, and excipient may be excluded from the immunogenic composition including a lipid-based delivery system.

In a further aspect, the immunogenic composition including a lipid-based delivery system further comprises a stabilizing agent. In some aspects, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In some aspects, 1, 2, 3, 4, 5, or more of the foregoing stabilizing agents may be excluded from the immunogenic compositions disclosed herein. In some aspects, the stabilizing agent is a disaccharide, or sugar. In one aspect, the stabilizing agent is sucrose. In another aspect, the stabilizing agent is trehalose. In a further aspect, the stabilizing agent is a combination of sucrose and trehalose. In some aspects, the total concentration of the stabilizing agent(s) in the composition is or is about 5% to about 10% w/v. For example, the total concentration of the stabilizing agent may or may not be equal to at least, at most, exactly, or between (inclusive or exclusive) of 1%, 2%, 3%, 4%, 5%, 6%, 7%, 8%, 9%, 10%, 11%, 12%, 13%, 14%, 15%, 16%, 17%, 18%, 19%, or 20% w/v or any range or value derivable therein. In some aspects, the stabilizing agent concentration includes, but is not limited to, a concentration of or of about 10 mg/mL to about 400 mg/mL, about 100 mg/mL to about 200 mg/mL, about 100 mg/mL to about 150 mg/mL, about 100 mg/mL to about 140 mg/mL, about 100 mg/mL to about 130 mg/mL, about 100 mg/mL to about 120 mg/mL, about 100 mg/mL to about 110 mg/mL, or about 100 mg/mL to about 105 mg/mL. In some aspects, the concentration of the stabilizing agent is or is not equal to at least, at most, exactly, or between (inclusive or exclusive) of 10 mg/mL, 20 mg/mL, 50 mg/mL, 100 mg/mL, 101 mg/mL, 102 mg/mL, 103 mg/mL, 104 mg/mL, 105 mg/mL, 106 mg/mL, 107 mg/mL, 108 mg/mL, 109 mg/mL, 110 mg/mL, 150 mg/mL, 200 mg/mL, 300 mg/mL, 400 mg/mL, or more.

In a further aspect, the mass amount of the stabilizing agent and the mass amount of the RNA are in a specific ratio. In one aspect, the ratio of the mass amount of the stabilizing agent and the RNA is no greater than 5000. In another aspect, the ratio of the mass amount of the stabilizing agent and the RNA is no greater than 2000. In another aspect, the ratio of the mass amount of the stabilizing agent and the RNA is no greater than 1000. In another aspect, the ratio of the mass amount of the stabilizing agent and the RNA is no greater than 500. In another aspect, the ratio of the mass amount of the stabilizing agent and the RNA is no greater than 100. In another aspect, the ratio of the mass amount of the stabilizing agent and the pharmaceutical substance is no greater than 50. In another aspect, the ratio of the mass amount of the stabilizing agent and the RNA is no greater than 10. In another aspect, the ratio of the mass amount of the stabilizing agent and the RNA is no greater than 1. In another aspect, the ratio of the mass amount of the stabilizing agent and the RNA is no greater than 0.5. In another aspect, the ratio of the mass amount of the stabilizing agent and the RNA is no greater than 0.1. In another aspect, the stabilizing agent and RNA comprise a mass ratio of or of about 200-2000 of the stabilizing agent:1 of the RNA.

In some aspects, the immunogenic composition including a lipid-based delivery system further comprises a buffer. Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, Tris hydrochloride (HCl), amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some aspects, 1, 2, 3, 4, 5, or more of the foregoing buffering agents may be excluded from the immunogenic compositions disclosed herein. In some aspects, the buffer is a HEPES buffer, a Tris buffer, and/or a PBS buffer. In one aspect, the buffer is Tris buffer. In another aspect, the buffer is a HEPES buffer. In a further aspect, the buffer is a PBS buffer. For example, the buffer concentration may or may not be equal to at least, at most, exactly, or between (inclusive or exclusive) of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. The buffer may be at a neutral pH, pH 6.5 to 8.5, pH 7.0 to pH 8.0, or pH 7.2 to pH 7.6. For example, the buffer may or may not be at least, at most, exactly, or between (inclusive or exclusive) of pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific aspects, the buffer is at pH 7.4.

In some aspects, the immunogenic composition including a lipid-based delivery system may further comprise a salt. Examples of salts include but not limited to sodium salts and/or potassium salts. In one aspect, the salt is a sodium salt. In a specific aspect, the sodium salt is sodium chloride. In one aspect, the salt is a potassium salt. In some aspects, the potassium salt comprises potassium chloride. In some aspects, any one or more of the foregoing salts may be excluded from the immunogenic compositions disclosed herein. The concentration of the salts in the composition may be or be about 70 mM to about 140 mM. For example, the salt concentration may or may not be equal to at least, at most, exactly, or between (inclusive or exclusive) of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM.

In some aspects, the salt concentration includes, but is not limited to, a concentration of or of about 1 mg/mL to about 100 mg/mL, about 1 mg/mL to about 50 mg/mL, about 1 mg/mL to about 40 mg/mL, about 1 mg/mL to about 30 mg/mL, about 1 mg/mL to about 20 mg/mL, about 1 mg/mL to about 10 mg/mL, or about 1 mg/mL to about 15 mg/mL. In some aspects, the concentration of the salt is or is not equal to at least, at most, exactly, or between (inclusive or exclusive) of 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, or more. The salt may be at a neutral pH, pH 6.5 to 8.5, pH 7.0 to pH 8.0, or pH 7.2 to pH 7.6. For example, the salt may or may not be at a pH equal to at least, at most, exactly, or between (inclusive or exclusive) of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5.

In some aspects, the immunogenic composition including a lipid-based delivery system further comprises a surfactant, a preservative, any other excipient, or a combination thereof. As used herein, “any other excipient” includes, but is not limited to, antioxidants, glutathione, EDTA, methionine, desferal, antioxidants, metal scavengers, and/or free radical scavengers. In one aspect, the surfactant, preservative, excipient or combination thereof is sterile water for injection (sWFI), bacteriostatic water for injection (BWFI), saline, dextrose solution, polysorbates, poloxamers, Triton, divalent cations, Ringer's lactate, amino acids, sugars, polyols, polymers, and/or cyclodextrins. In some aspects, 1, 2, 3, 4, 5, or more of the foregoing excipients may be excluded from the immunogenic compositions disclosed herein.

Further examples of excipients, which refer to ingredients in the immunogenic compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, and/or colorants. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyl oleate), dispersion media, coatings, surfactants, antioxidants, preservatives (e.g., antibacterial and/or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato; and celluloses such as microcrystalline cellulose. The amount of diluent in the composition may range from or from about 10% to about 90% by weight of the total composition, e.g., from or from about 25% to about 75%, about 30% to about 60% by weight, or about 12% to about 60%. Preservatives for use in the compositions disclosed herein include but are not limited to benzalkonium chloride, chlorobutanol, paraben and thimerosal. In some aspects, 1, 2, 3, 4, 5, or more of the foregoing excipients may be excluded from the immunogenic compositions disclosed herein.

The pH and exact concentration of the various components in the immunogenic composition including a lipid-based delivery system are adjusted according to well-known parameters. The use of such media and agents for pharmaceutical active substances is well known in the art. Except insofar as any conventional media or agent is incompatible with the active ingredients, its use in immunogenic, prophylactic and/or therapeutic compositions is contemplated.

In one aspect, a pharmaceutical composition comprises an RNA molecule encoding a polypeptide as disclosed herein that is complexed with, encapsulated in, and/or formulated with one or more lipids to form RNA-LNPs. In some aspects, the RNA-LNP composition is a liquid. In some aspects, the RNA-LNP composition is frozen. In some aspects, the RNA-LNP composition is lyophilized. In some aspects, a RNA-LNP composition comprises a RNA polynucleotide molecule encoding a polypeptide as disclosed herein, encapsulated in LNPs with a lipid composition of a cationic lipid, a PEGylated lipid (e.g. PEG-lipid), and one or more structural lipids (e.g., a neutral lipid). In some aspects, any one or more of the foregoing lipids may be excluded from the LNPs of the pharmaceutical compositions disclosed herein.

In some aspects, a RNA-LNP composition comprises a cationic lipid. The cationic lipid may comprise any one or more cationic lipids disclosed herein.

The cationic lipid may comprise any one or more cationic lipids disclosed herein. In specific aspects, the cationic lipid comprises ((4-hydroxybutyl) azanediyl)bis(hexane-6,1-diyl)bis(2-hexyldecanoate) (ALC-0315). In some aspects, the cationic lipid (e.g., ALC-0315) is or is not included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the cationic lipid (e.g., ALC-0315) is or is not included in the composition at a concentration of at least, at most, between (inclusive or exclusive) of, or exactly 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the cationic lipid (e.g., ALC-0315) is included in the composition at a concentration of at least 0.4, at least 0.45, at least 0.5, at least 0.55, at least 0.6, at least 0.65, at least 0.7, at least 0.75, at least 0.8, at least 0.85, at least 0.9, at least 0.95, or at least 1 mg/mL. In some aspects, the cationic lipid (e.g., ALC-0315) is included in the composition at a concentration of between 0.4 and 0.5, between 0.5 and 0.6, between 0.6 and 0.7, between 0.7 and 0.8, between 0.8 and 0.9, or between 0.9 and 1. In some aspects, the cationic lipid (e.g., ALC-0315) is included in the composition at a concentration of between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95, or between 0.95 and 1 mg/mL.

In specific aspects, the cationic lipid (e.g., ALC-0315) is included in the composition at a concentration of 0.8 to 0.95 mg/mL. In specific aspects, the cationic lipid (e.g., ALC-0315) is included in the composition at a concentration of or of about 0.8 to 0.9 mg/mL. In specific aspects, the cationic lipid (e.g., ALC-0315) is included in the composition at a concentration of or of about 0.85 to 0.9 mg/mL. In specific aspects, the cationic lipid (e.g., ALC-0315) is or is not included in the composition at a concentration of or of at least, at most, exactly, between (inclusive or exclusive) any two of, or about 0.8, 0.81, 0.82, 0.83, 0.84, 0.85, 0.86, 0.87, 0.88, 0.89, 0.9, 0.91, 0.92, 0.93, 0.94, or 0.95 mg/mL. In specific aspects, the cationic lipid (e.g., ALC-0315) is included in the composition at a concentration of or of about 0.86 mg/mL. Concentrations for lyophilized compositions are determined post-reconstitution.

In some aspects, a RNA-LNP composition further comprises a PEGylated lipid (e.g., PEG-lipid).

The PEGylated lipid may comprise any one or more PEGylated lipids disclosed herein. In specific aspects, the PEGylated lipid comprises 2-[(polyethylene glycol)-2000]-N,N-ditetradecylacetamide (ALC-0159). In some aspects, the PEGylated lipid (e.g., ALC-0159) is or is not included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the PEGylated lipid (e.g., ALC-0159) is or is not included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the PEGylated lipid (e.g., ALC-0159) is included in the composition at a concentration of at least 0.01, at least 0.05, at least 0.1, at least 0.15, at least 0.2, at least 0.25 mg/mL, at least 0.3 mg/mL, at least 0.35 mg/mL, at least 0.4 mg/mL, at least 0.45 mg/mL, or at least 0.5 mg/mL. In some aspects, the PEGylated lipid (e.g., ALC-0159) is included in the composition at a concentration of between 0.01 and 0.05, between 0.05 and 0.1, between 0.1 and 0.15, between 0.15 and 0.2, or between 0.2 and 0.25 mg/mL.

In specific aspects, the PEGylated lipid (e.g., ALC-0159) is included in the composition at a concentration of or of about 0.05 to 0.15 mg/mL. In specific aspects, the PEGylated lipid (e.g., ALC-0159) is included in the composition at a concentration of or of about 0.10 to 0.15 mg/mL. In specific aspects, the PEGylated lipid (e.g., ALC-0159) is or is not included in the composition at a concentration of or of at least, at most, exactly, between (inclusive or exclusive) any two of, or about 0.05, 0.06, 0.07, 0.08, 0.09, 0.1, 0.11, 0.12, 0.13, 0.14, or 0.15 mg/mL. In specific aspects, the PEGylated lipid (e.g., ALC-0159) is included in the composition at a concentration of or of about 0.11 mg/mL Concentrations for lyophilized compositions are determined post-reconstitution.

In some aspects, a RNA-LNP composition further comprises one or more structural lipids.

The one or more structural lipids may comprise any one or more structural lipids disclosed herein. In specific aspects, the one or more structural lipids comprise a neutral lipid and a steroid or steroid analog. In specific aspects, the one or more structural lipids comprise 1,2-distearoyl-sn-glycero-3-phosphocholine (DSPC) and cholesterol. In some aspects, the one or more structural lipids (e.g., DSPC and cholesterol) are or are not included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the one or more structural lipids (e.g., DSPC and cholesterol) are or are not included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the one or more structural lipids (e.g., DSPC and cholesterol) are included in the composition at a concentration of at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the one or more structural lipids (e.g., DSPC and cholesterol) are included in the composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95, or between 0.95 and 1 mg/mL.

In specific aspects, the one or more structural lipids include DSPC, and the DSPC is included in the composition at a concentration of or of about 0.1 to 0.25 mg/mL. In specific aspects, the one or more structural lipids include DSPC, and the DSPC is included in the composition at a concentration of or of about 0.15 to 0.25 mg/mL. In specific aspects, the one or more structural lipids include DSPC, and the DSPC is or is not included in the composition at a concentration of or of at least, at most, exactly, between (inclusive or exclusive) any two of, or about 0.1, 0.11, 0.12, 0.13, 0.14, 0.15, 0.16, 0.17, 0.18, 0.19, 0.2, 0.21, 0.22, 0.23, 0.24, or 0.25 mg/mL. In specific aspects, the DSPC is included in the composition at a concentration of or of about 0.19 mg/mL.

In specific aspects, the one or more structural lipids include cholesterol, and the cholesterol is included in the composition at a concentration of or of about 0.3 to 0.45 mg/mL. In specific aspects, the one or more structural lipids include cholesterol, and the cholesterol is included in the composition at a concentration of or of about 0.3 to 0.4. In specific aspects, the one or more structural lipids include cholesterol, and the cholesterol is included in the composition at a concentration of or of about 0.35 to 0.45. In specific aspects, the one or more structural lipids include cholesterol, and the cholesterol is or is not included in the composition at a concentration of or of at least, at most, exactly, between (inclusive or exclusive) any two of, or about 0.30, 0.31, 0.32, 0.33, 0.34, 0.35, 0.36, 0.37, 0.38, 0.39, 0.40, 0.41, 0.42, 0.43, 0.44, or 0.45 mg/mL. In specific aspects, the cholesterol is included in the composition at a concentration of and/or of about 0.37 mg/mL. Concentrations for lyophilized compositions are determined post-reconstitution.

In some aspects, the RNA-LNP composition further comprises one or more buffers and stabilizing agents, and optionally, salt diluents. Thus, in some aspects, the RNA-LNP composition comprises an cationic lipid, a PEGylated lipid, one or more structural lipids, one or more buffers, a stabilizing agent, and optionally, a salt diluent. In some aspects, 1, 2, 3, or more of the foregoing elements are excluded from the RNA-LNP composition.

In some aspects, a RNA-LNP composition comprises one or more buffers.

The one or more buffers may comprise any one or more buffering agents disclosed herein. In specific aspects, the composition comprises a Tris buffer comprising at least a first buffer and a second buffer. In some aspects, the first buffer is tromethamine. In some aspects, the second buffer is Tris hydrochloride (HCl). In some aspects, the first buffer and second buffer of the Tris buffer (e.g., tromethamine and Tris HCl) are or are not included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. Concentrations for lyophilized compositions are determined post-reconstitution.

In some aspects, the RNA-LNP composition is a liquid composition comprising a Tris buffer.

In some aspects, the Tris buffer comprises a first buffer. In some aspects, the first buffer is tromethamine. In some aspects, the first buffer (e.g., tromethamine) is or is not included in the liquid composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the liquid composition at a concentration of at least 0.1, at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95, or at least 1 mg/mL. In some aspects, the first buffer (e.g., tromethamine) is included in the liquid composition at a concentration of between 0.05 and 0.15, between 0.15 and 0.25, between 0.25 and 0.35, between 0.35 and 0.45, between 0.45 and 0.55, between 0.55 and 0.65, between 0.65 and 0.75, between 0.75 and 0.85, or between 0.85 and 0.95. In some aspects, the first buffer (e.g., tromethamine) is included in the liquid composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95, or between 0.95 and 1 mg/mL.

In specific aspects, the first buffer (e.g., tromethamine) is included in the liquid composition at a concentration of or of about 0.1 to 0.3 mg/mL. In specific aspects, the first buffer (e.g., tromethamine) is included in the liquid composition at a concentration of or of about 0.15 to 0.25 mg/mL. In specific aspects, the first buffer (e.g., tromethamine) is or is not included in the liquid composition at a concentration of or of at least, at most, exactly, between (inclusive or exclusive) any two of, or about 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.3 mg/mL. In specific aspects, the first buffer (e.g., tromethamine) is included in the liquid composition at a concentration of or of about 0.20 mg/mL.

In some aspects, the RNA-LNP composition is a liquid composition comprising a Tris buffer comprising a second buffer.

In some aspects, the second buffer comprises Tris HCl. In some aspects, the second buffer (e.g., Tris HCl) is or is not included in the liquid composition at a concentration of at least, at most, between (inclusive or exclusive) of, or exactly 0.5, 0.55, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5 mg/mL. In some aspects, the second buffer (e.g., Tris HCl) is included in the liquid composition at a concentration of at least 0.5, at least 0.55, at least 0.6, at least 0.65, at least 0.7, at least 0.75, at least 0.8, at least 0.85, at least 0.9, at least 0.95, at least 1, at least 1.05, at least 1.10, at least 1.15, at least 1.20, at least 1.25, at least 1.30, at least 1.35, at least 1.40, at least 1.45, or at least 1.50 mg/mL. In some aspects, the second buffer (e.g., Tris HCl) is included in the liquid composition at a concentration of between 0.5 and 0.6, between 0.6 and 0.7, between 0.7 and 0.8, between 0.8 and 0.9, between 0.9 and 1, between 1 and 1.10, between 1.10 and 1.20, between 1.20 and 1.30, between 1.30 and 1.40, or between 1.40 and 1.50 mg/mL.

In specific aspects, the second buffer (e.g., Tris HCl) is included in the liquid composition at a concentration of or of about 1.25 to 1.40 mg/mL. In specific aspects, the second buffer (e.g., Tris HCl) is included in the liquid composition at a concentration of or of about 1.30 to 1.40 mg/mL. In specific aspects, the second buffer (e.g., Tris HCl) is or is not included in the liquid composition at a concentration of or of at least, at most, exactly, between (inclusive or exclusive) any two of, or about 1.25, 1.26, 1.27, 1.28, 1.29, 1.30, 1.31, 1.32, 1.33, 1.34, or 1.35, 1.36, 1.37, 1.38, 1.39, or 1.40 mg/mL. In specific aspects, the second buffer (e.g., Tris HCl) is included in the liquid composition at a concentration of or of about 1.32 mg/mL.

In some aspects, the RNA-LNP composition is a lyophilized composition comprising a Tris buffer. In some aspects, the Tris buffer comprises a first buffer. In some aspects, the first buffer is tromethamine. In some aspects, the first buffer (e.g., tromethamine) is or is not included in the lyophilized composition at a concentration, after reconstitution, of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the lyophilized composition at a concentration, after reconstitution, of at least 0.01, of at least 0.05, of at least 0.1, of at least 0.15, of at least 0.2, of at least 0.25, of at least 0.3, of at least 0.35, of at least 0.4, of at least 0.45, or of at least 0.5 mg/mL. In some aspects, the first buffer (e.g., tromethamine (Tris base)) is included in the lyophilized composition at a concentration, after reconstitution, of between 0.01 and 0.05, between 0.05 and 0.1, between 0.1 and 0.15, between 0.15 and 0.2, between 0.2 and 0.25 mg/mL, between 0.25 and 0.3 mg/mL, between 0.3 and 0.35 mg/mL, between 0.35 and 0.4 mg/mL, between 0.4 and 0.45 mg/mL, or between 0.45 and 0.5 mg/mL.

In specific aspects, the first buffer (e.g., tromethamine) is included in the lyophilized composition at a concentration, after reconstitution, of or of about 0.01 and 0.15 mg/mL. In specific aspects, the first buffer (e.g., tromethamine) is included in the lyophilized composition at a concentration, after reconstitution, of or of about 0.01 and 0.10 mg/mL. In specific aspects, the first buffer (e.g., tromethamine) is included in the lyophilized composition at a concentration, after reconstitution, of or of about 0.05 and 0.15 mg/mL. In specific aspects, the first buffer (e.g., tromethamine) is or is not included in the lyophilized composition at a concentration, after reconstitution, of or of at least, at most, exactly, between (inclusive or exclusive) any two of, or about 0.01, 0.02, 0.03, 0.04, 0.05, 0.06, 0.07, 0.08, 0.09, 0.1, 0.11, 0.12, 0.13, 0.14, or 0.15 mg/mL. In specific aspects, the first buffer (e.g., tromethamine) is included in the lyophilized composition at a concentration, after reconstitution, of or of about 0.09 mg/mL.

In some aspects, the RNA-LNP composition is a lyophilized composition comprising a Tris buffer comprising a second buffer. In some aspects, the second buffer comprises Tris HCl. In some aspects, the second buffer (e.g., Tris HCl) is or is not included in the lyophilized composition at a concentration, after reconstitution, of at least, at most, between (inclusive or exclusive) of, or exactly 0.1, 0.15, 0.2, 0.25, 0.3, 0.35, 0.4, 0.45, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the second buffer (e.g., Tris HCl) is included in the lyophilized composition at a concentration, after reconstitution, of at least 0.1, at least 0.2, at least 0.3, at least 0.4, at least 0.5, at least 0.6, at least 0.7, at least 0.8, at least 0.9, or at least 1 mg/mL. In some aspects, the second buffer (e.g., Tris HCl) is included in the lyophilized composition at a concentration, after reconstitution, of between 0.1 and 0.2, between 0.2 and 0.3, between 0.3 and 0.4, between 0.4 and 0.5, between 0.5 and 0.6, between 0.6 and 0.7, between 0.7 and 0.8, between 0.8 and 0.9, or between 0.9 and 1 mg/mL.

In specific aspects, the second buffer (e.g., Tris HCl) is included in the lyophilized composition at a concentration, after reconstitution, of or of about 0.5 and 0.65 mg/mL. In specific aspects, the second buffer (e.g., Tris HCl) is included in the lyophilized composition at a concentration, after reconstitution, of or of about 0.5 and 0.6 mg/mL. In specific aspects, the second buffer (e.g., Tris HCl) is included in the lyophilized composition at a concentration, after reconstitution, of or of about 0.55 and 0.65 mg/mL. In specific aspects, the second buffer (e.g., Tris HCl) is or is not included in the lyophilized composition at a concentration, after reconstitution, of or of at least, at most, exactly, between (inclusive or exclusive) any two of, or about 0.5, 0.51, 0.52, 0.53, 0.54, 0.55, 0.56, 0.57, 0.58, 0.59, 0.6, 0.61, 0.62, 0.63, 0.64, or 0.65 mg/mL. In specific aspects, the second buffer (e.g., Tris HCl) is included in the lyophilized composition at a concentration, after reconstitution, of or of about 0.57 mg/mL.

In some aspects, a RNA-LNP composition comprises a stabilizing agent. The stabilizing agent may comprise any one or more stabilizing agents disclosed herein. In some aspects, the stabilizing agent also functions as a cryoprotectant. In specific aspects, the stabilizing agent comprises sucrose. In some aspects, the stabilizing agent (e.g., sucrose) is or is not included in the composition at a concentration of at least, at most, between (inclusive or exclusive) of, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 ng/μg/mg per mL.

In some aspects, the RNA-LNP composition is a liquid composition, and the stabilizing agent (e.g., sucrose) is or is not included in the liquid composition at a concentration of at least, at most, between (inclusive or exclusive) of, or exactly 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 mg/mL. In some aspects, the stabilizing agent (e.g., sucrose) is included in the liquid composition at a concentration of at least 70, at least 75, at least 80, at least 85, at least 90, at least 95, at least 100, at least 105, at least 110, at least 115, at least 120, at least 125, or at least 130 mg/mL. In some aspects, the stabilizing agent (e.g., sucrose) is included in the liquid composition at a concentration of between 70 and 80, between 80 and 90, between 90 and 100, between 100 and 110, between 110 and 120, or between 120 and 130 mg/mL.

In specific aspects, the stabilizing agent (e.g., sucrose) is included in the liquid composition at a concentration of or of about 95 to 110 mg/mL. In specific aspects, the stabilizing agent (e.g., sucrose) is included in the liquid composition at a concentration of or of about 95 to 105 mg/mL. In specific aspects, the stabilizing agent (e.g., sucrose) is included in the liquid composition at a concentration of or of about 100 to 110 mg/mL. In specific aspects, the stabilizing agent (e.g., sucrose) is or is not included in the liquid composition at a concentration of or of at least, at most, exactly, between (inclusive or exclusive) any two of, or about 95, 96, 97, 98, 99, 100, 101, 102, 103, 104, 105, 106, 107, 108, 109, or 110 mg/mL. In specific aspects, the stabilizing agent (e.g., sucrose) is included in the liquid composition at a concentration of or of about 103 mg/mL.

In some aspects, the RNA-LNP composition is a lyophilized composition, and the stabilizing agent (e.g., sucrose) is or is not included in the lyophilized composition at a concentration, after reconstitution, of at least, at most, between (inclusive or exclusive) of, or exactly 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 mg/mL. In some aspects, the stabilizing agent (e.g., sucrose) is included in the lyophilized composition at a concentration, after reconstitution, of at least 20, at least 25, at least 30, at least 35, at least 40, at least 45, at least 50, at least 55, at least 60, at least 65, at least 70, at least 75, or at least 80 mg/mL. In some aspects, the stabilizing agent (e.g., sucrose) is included in the lyophilized composition at a concentration, after reconstitution, of between 20 to 30, between 30 to 40, between 40 to 50, between 50 to 60, between 60 to 70, or between 70 to 80 mg/mL.

In specific aspects, the stabilizing agent (e.g., sucrose) is included in the lyophilized composition at a concentration, after reconstitution, of or of about 35 to 50 mg/mL. In specific aspects, the stabilizing agent (e.g., sucrose) is included in the lyophilized composition at a concentration, after reconstitution, of or of about 35 to 45 mg/mL. In specific aspects, the stabilizing agent (e.g., sucrose) is included in the lyophilized composition at a concentration, after reconstitution, of or of about 40 to 50 mg/mL. In specific aspects, the stabilizing agent (e.g., sucrose) is or is not included in the lyophilized composition at a concentration, after reconstitution, of or of at least, at most, exactly, between (inclusive or exclusive) any two of, or about 35, 36, 37, 38, 39, 40, 41, 42, 43, 44, 45, 46, 47, 48, 49, or 50 mg/mL. In specific aspects, the stabilizing agent (e.g., sucrose) is included in the lyophilized composition at a concentration, after reconstitution, of or of about 44/mL.

In some aspects, lyophilized compositions are reconstituted in a suitable carrier and/or diluent. The carrier and/or diluent may comprise any one or more carriers and/or diluents disclosed herein. In specific aspects, the carrier and/or diluent comprises a salt diluent, such as sodium chloride (NaCl) (e.g., saline, e.g., physiological or normal saline). The sodium chloride may comprise 0.9% sodium chloride for injection. In some aspects, the lyophilized compositions are or are not reconstituted in at least, at most, between (inclusive or exclusive) of, or exactly 0.1, 0.2, 0.3, 0.4, 0.5, 0.6, 0.7, 0.8, 0.9, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1 mL of saline. In some aspects, the lyophilized compositions are reconstituted in at least 0.1, at least 0.2, at least 0.3, at least 0.4, at least 0.5, at least 0.6, at least 0.7, at least 0.8, at least 0.9, or at least 1 mL of sodium chloride.

In specific aspects, the lyophilized compositions are reconstituted in or in about 0.6 to 0.75 mL of sodium chloride/saline. In specific aspects, the lyophilized compositions are reconstituted in or in about 0.65 to 0.75 mL of sodium chloride/saline. In specific aspects, the lyophilized compositions are or are not reconstituted in or in at least, at most, exactly, between (inclusive or exclusive) any two of, or about 0.6, 0.61, 0.62, 0.63, 0.64, 0.65, 0.66, 0.67, 0.68, 0.69, 0.7, 0.71, 0.72, 0.73, 0,74, or 0.75 mL of sodium chloride/saline.

In some aspects, the salt diluent (e.g., NaCl) is or is not included in the lyophilized composition at a concentration, after reconstitution, of at least, at most, between (inclusive or exclusive) of, or exactly 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or 50 ng/μg/mg per mL. In some aspects, the salt diluent (e.g., NaCl) is or is not included in the lyophilized composition at a concentration, after reconstitution, of in at least, at most, between (inclusive or exclusive) of, or exactly 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 mg/mL. In some aspects, the salt diluent (e.g., NaCl) is included in the lyophilized composition at a concentration, after reconstitution, of at least 1, at least 2, at least 3, at least 4, at least 5, at least 6, at least 7, at least 8, at least 9, at least 10, at least 11, at least 12, at least 13, at least 14, at least 15, or at least 20 mg/mL.

In specific aspects, the salt diluent (e.g., NaCl) is included in the lyophilized composition at a concentration, after reconstitution, of between or between about 5 and 15 mg/mL. In some aspects, the salt diluent (e.g., NaCl) is included in the lyophilized composition at a concentration, after reconstitution, of between or between about 5 and 10 mg/mL. In specific aspects, the salt diluent (e.g., NaCl) is or is not included in the lyophilized composition at a concentration, after reconstitution, of or of at least, at most, exactly, between (inclusive or exclusive) any two of, or about 5, 6, 7, 8, 9, 10, 11, 12, 13, 14, or 15 mg/mL. In specific aspects, the salt diluent (e.g., NaCl) is included in the lyophilized composition at a concentration, after reconstitution, of or of about 9 mg/mL.

The pH of the RNA-LNP composition may or may not be at least, at most, exactly, or between (inclusive or exclusive) of pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5, or any range or value derivable therein. In some aspects, the RNA-LNP composition is at a pH of at least 6.5, at least 7.0, at least 7.5, at least 8.0, or at least 8.5. In specific aspects, the RNA-LNP composition is at a pH between 6.0 and 7.5, between 6.5 and 7.5, between 7.0 and 8.0, between and 7.5 and 8.5. In specific aspects, the RNA-LNP composition is between 7.0 and 8.0. In specific aspects, the RNA-LNP composition is or is not at least, at most, exactly, between (inclusive or exclusive) any two of, or about pH 7.0, 7.1, 7.2, 7.3, 7.4, 7.5, 7.6, 7.7, 7.8, 7.9, or 8.0. In specific aspects, the RNA-LNP composition is at or at about pH 7.4. In some aspects, sodium hydroxide buffer may be used for a buffer pH adjustment.

In specific aspects, a RNA-LNP composition comprises a RNA polynucleotide encoding a polypeptide as disclosed herein, encapsulated in LNPs with a lipid composition of an cationic lipid at a concentration of or of about 0.8 to 0.95 mg/mL, a PEGylated lipid at a concentration of or of about 0.05 to 0.15 mg/mL, a first structural lipid at a concentration of or of about 0.1 to 0.25 mg/mL, and a second structural lipid at a concentration of or of about 0.3 to 0.45 mg/mL. In some aspects, one or more of the foregoing elements may be excluded from the RNA-LNP composition.

In specific aspects, a RNA-LNP composition comprises a RNA polynucleotide encoding a polypeptide as disclosed herein, encapsulated in LNPs with a lipid composition of ALC-0315 at a concentration of or of about 0.8 to 0.95 mg/mL, ALC-0159 at a concentration of or of about 0.05 to 0.15 mg/mL, DSPC at a concentration of or of about 0.1 to 0.25 mg/mL, and cholesterol at a concentration of about 0.3 to 0.45 mg/mL. In some aspects, one or more of the foregoing elements may be excluded from the RNA-LNP composition.

In specific aspects, the RNA-LNP composition is a liquid RNA-LNP composition, and the liquid RNA-LNP composition further comprises a buffer composition comprising a first buffer at a concentration of or of about 0.15 to 0.3 mg/mL, a second buffer at a concentration of or of about 1.25 to 1.4 mg/mL, and a stabilizing agent at a concentration of or of about 95 to 110 mg/mL. In specific aspects, the RNA-LNP composition is a liquid RNA-LNP composition, and the liquid RNA-LNP composition further comprises a Tris buffer composition comprising tromethamine at a concentration of or of about 0.1 to 0.3 mg/mL, Tris HCl at a concentration of or of about 1.25 to 1.4 mg/mL, and sucrose at a concentration of or of about 95 to 110 mg/mL. In some aspects, one or more of the foregoing elements may be excluded from the RNA-LNP composition.

Thus, in specific aspects, a liquid RNA-LNP composition comprises an cationic lipid at a concentration of or of about 0.8 to 0.95 mg/mL, a PEGylated lipid at a concentration of or of about 0.05 to 0.15 mg/mL, a first structural lipid at a concentration of or of about 0.1 to 0.25 mg/mL, a second structural lipid at a concentration of or of about 0.3 to 0.45 mg/mL, and further comprises a first buffer at a concentration of or of about 0.1 to 0.3 mg/mL, a second buffer at a concentration of or of about 1.25 to 1.4 mg/mL, and a stabilizing agent at a concentration of or of about 95 to 110 mg/mL. In some aspects, one or more of the foregoing elements may be excluded from the RNA-LNP composition.

Thus, in specific aspects, a liquid RNA-LNP composition comprises ALC-0315 at a concentration of or of about 0.8 to 0.95 mg/mL, ALC-0159 at a concentration of or of about 0.05 to 0.15 mg/mL, DSPC at a concentration of or of about 0.1 to 0.25 mg/mL, cholesterol at a concentration of or of about 0.3 to 0.45 mg/mL, and further comprises a Tris buffer composition comprising tromethamine at a concentration of or of about 0.1 to 0.3 mg/mL, Tris HCl at a concentration of or of about 1.25 to 1.4 mg/mL, and sucrose at a concentration of or of about 95 to 110 mg/mL. In some aspects, one or more of the foregoing elements may be excluded from the RNA-LNP composition.

In specific aspects, the RNA-LNP composition is a lyophilized RNA-LNP composition, and the lyophilized RNA-LNP composition further comprises (after reconstitution) a first buffer at a concentration of or of about 0.01 and 0.15 mg/mL, a second buffer at a concentration of or of about 0.5 and 0.65 mg/mL, a stabilizing agent at a concentration of or of about 35 to 50 mg/mL, and a salt diluent at a concentration of between or between about 5 and 15 mg/mL. In some aspects, one or more of the foregoing elements may be excluded from the RNA-LNP composition.

In specific aspects, the RNA-LNP composition is a lyophilized RNA-LNP composition, and the lyophilized RNA-LNP composition further comprises (after reconstitution) a Tris buffer composition comprising tromethamine at a concentration of or of about 0.01 and 0.15 mg/mL, Tris HCl at a concentration of or of about 0.5 and 0.65 mg/mL, sucrose at a concentration of or of about 35 to 50 mg/mL, and sodium chloride (NaCl) at a concentration of or of about 5 to 15 mg/mL. In some aspects, one or more of the foregoing elements may be excluded from the RNA-LNP composition.

Thus, in specific aspects, a lyophilized RNA-LNP composition comprises (after reconstitution) a cationic lipid at a concentration of or of about 0.8 to 0.95 mg/mL, a PEGylated lipid at a concentration of or of about 0.05 to 0.15 mg/mL, a first structural lipid at a concentration of or of about 0.1 to 0.25 mg/mL, a second structural lipid at a concentration of or of about 0.3 to 0.45 mg/mL, and further comprises a first buffer at a concentration of or of about 0.01 and 0.15 mg/mL, a second buffer at a concentration of or of about 0.5 and 0.65 mg/mL, a stabilizing agent at a concentration of or of about 35 to 50 mg/mL, and a salt diluent at a concentration of or of about 5 to 15 mg/mL. In specific aspects, the lyophilized compositions are reconstituted in 0.6 to 0.75 mL of the salt diluent. In some aspects, one or more of the foregoing elements may be excluded from the RNA-LNP composition.

Thus, in some aspects, a lyophilized RNA-LNP composition comprises (after reconstitution) ALC-0315 at a concentration of or of about 0.8 to 0.95 mg/mL, ALC-0159 at a concentration of or of about 0.05 to 0.15 mg/mL, DSPC at a concentration of or of about 0.1 to 0.25 mg/mL, cholesterol at a concentration of or of about 0.3 to 0.45 mg/mL, and further comprises tromethamine at a concentration of or of about 0.01 and 0.15 mg/mL, Tris HCl at a concentration of or of about 0.5 and 0.65 mg/mL, sucrose at a concentration of or of about 35 to 50 mg/mL, and NaCl at a concentration of or of about 5 to 15 mg/mL. In specific aspects, the lyophilized compositions are reconstituted in 0.6 to 0.75 mL of NaCl (saline). In some aspects, one or more of the foregoing elements may be excluded from the RNA-LNP composition.

Concentrations in the lyophilized RNA-LNP composition above are determined post-reconstitution.

In some aspects, a RNA-LNP composition (pre-lyophilization) comprises a cationic lipid at a concentration of or of about 1.0 to 3.0 mg/mL, a PEGylated lipid at a concentration of or of about 0.10 to 0.35 mg/mL, a first structural lipid at a concentration of or of about 0.4 to 0.55 mg/mL, a second structural lipid at a concentration of or of about 0.85 to 1.0 mg/mL, and further comprises a first buffer at a concentration of or of about 0.1 and 0.3 mg/mL, a second buffer at a concentration of or of about 1.25 and 1.40 mg/mL, a stabilizing agent at a concentration of or of about 95 to 110 mg/mL. In some aspects, one or more of the foregoing elements may be excluded from the RNA-LNP composition.

Thus, in some aspects, a RNA-LNP composition (pre-lyophilization) comprises ALC-0315 at a concentration of or of about 1.0 to 3.0 mg/mL, ALC-0159 at a concentration of or of about 0.10 to 0.35 mg/mL, DSPC at a concentration of or of about 0.4 to 0.55 mg/mL, cholesterol at a concentration of or of about 0.85 to 1.0 mg/mL, and further comprises tromethamine at a concentration of or of about 0.1 and 0.3 mg/mL, Tris HCl at a concentration of or of about 1.25 and 1.40 mg/mL, sucrose at a concentration of or of about 95 to 110 mg/mL. In some aspects, one or more of the foregoing elements may be excluded from the RSV RNA-LNP composition.

The RNA-LNP compositions further comprise RNA described herein encapsulated in LNPs.

In specific aspects, a RNA-LNP composition is a liquid RNA-LNP composition comprising a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, encapsulated in LNPs with a lipid composition of an cationic lipid at a concentration of or of about 0.8 to 0.95 mg/mL, a PEGylated lipid at a concentration of or of about 0.05 to 0.15 mg/mL, a first structural lipid at a concentration of or of about 0.1 to 0.25 mg/mL, and a second structural lipid at a concentration of or of about 0.3 to 0.45 mg/mL, and further comprising a buffer composition comprising a first buffer at a concentration of or of about 0.15 to 0.3 mg/mL, a second buffer at a concentration of or of about 1.25 to 1.4 mg/mL, and a stabilizing agent at a concentration of or of about 95 to 110 mg/mL. In some aspects, one or more of the foregoing elements may be excluded from the RNA-LNP composition.

In specific aspects, a liquid RNA-LNP composition comprises a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, and more preferably of or of about 0.06 mg/mL, encapsulated in LNPs with a lipid composition of ALC-0315 at a concentration of or of about 0.8 to 0.95 mg/mL, ALC-0159 at a concentration of or of about 0.05 to 0.15 mg/mL, DSPC at a concentration of or of about 0.1 to 0.25 mg/mL, and cholesterol at a concentration of or of about 0.3 to 0.45 mg/mL, and further comprising a Tris buffer composition comprising tromethamine at a concentration of or of about 0.1 to 0.3 mg/mL, Tris HCl at a concentration of or of about 1.25 to 1.4 mg/mL, and sucrose at a concentration of or of about 95 to 110 mg/mL. In some aspects, one or more of the foregoing elements may be excluded from the RNA-LNP composition.

In specific aspects, the RNA-LNP composition is a lyophilized RNA-LNP composition comprising a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, encapsulated in LNPs with a lipid composition of a cationic lipid at a concentration of or of about 0.8 to 0.95 mg/mL, a PEGylated lipid at a concentration of or of about 0.05 to 0.15 mg/mL, a first structural lipid at a concentration of or of about 0.1 to 0.25 mg/mL, and a second structural lipid at a concentration of or of about 0.3 to 0.45 mg/mL, and further comprising a first buffer at a concentration of or of about 0.01 and 0.15 mg/mL, a second buffer at a concentration of or of about 0.5 and 0.65 mg/mL, a stabilizing agent at a concentration of or of about 35 to 50 mg/mL, and a salt diluent at a concentration of 5 to 15 mg/mL. In some aspects, one or more of the foregoing elements may be excluded from the RNA-LNP composition. In specific aspects, the lyophilized compositions are reconstituted in 0.6 to 0.75 mL of the salt diluent. Concentrations in the lyophilized RNA-LNP composition are determined post-reconstitution.

In specific aspects, a lyophilized RNA-LNP composition comprises a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, and more preferably of or of about 0.06 mg/mL, encapsulated in LNPs with a lipid composition of ALC-0315 at a concentration of or of about 0.8 to 0.95 mg/mL, ALC-0159 at a concentration of or of about 0.05 to 0.15 mg/mL, DSPC at a concentration of or of about 0.1 to 0.25 mg/mL, and cholesterol at a concentration of or of about 0.3 to 0.45 mg/mL, and further comprising tromethamine at a concentration of or of about 0.01 and 0.15 mg/mL, Tris HCl at a concentration of or of about 0.5 and 0.65 mg/mL, sucrose at a concentration of or of about 35 to 50 mg/mL, and NaCl at a concentration of or of about 5 to 15 mg/mL. In some aspects, one or more of the foregoing elements may be excluded from the RNA-LNP composition. In specific aspects, the lyophilized compositions are reconstituted in 0.6 to 0.75 mL of the NaCl diluent (saline). Concentrations in the lyophilized RNA-LNP composition are determined post-reconstitution.

In some aspects, a RNA-LNP composition (pre-lyophilization) comprises a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, encapsulated in LNPs with a lipid composition of a cationic lipid at a concentration of or of about 1.0 to 3.0 mg/mL, a PEGylated lipid at a concentration of or of about 0.10 to 0.35 mg/mL, a first structural lipid at a concentration of or of about 0.4 to 0.55 mg/mL, a second structural lipid at a concentration of or of about 0.85 to 1.0 mg/mL, and further comprises a first buffer at a concentration of or of about 0.1 and 0.3 mg/mL, a second buffer at a concentration of or of about 1.25 and 1.40 mg/mL, a stabilizing agent at a concentration of or of about 95 to 110 mg/mL. In some aspects, one or more of the foregoing elements may be excluded from the RNA-LNP composition.

Thus, in some aspects, a RNA-LNP composition (pre-lyophilization) comprises a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, and more preferably 0.15 mg/mL, encapsulated in LNPs with a lipid composition of comprises ALC-0315 at a concentration of or of about 1.0 to 3.0 mg/mL, ALC-0159 at a concentration of or of about 0.10 to 0.35 mg/mL, DSPC at a concentration of or of about 0.4 to 0.55 mg/mL, cholesterol at a concentration of or of about 0.85 to 1.0 mg/mL, and further comprises tromethamine at a concentration of or of about 0.1 and 0.3 mg/mL, Tris HCl at a concentration of or of about 1.25 and 1.40 mg/mL, sucrose at a concentration of or of about 95 to 110 mg/mL. In some aspects, one or more of the foregoing elements may be excluded from the RNA-LNP composition.

In some aspects, the liquid RNA-LNP immunogenic composition comprises an RNA molecule/polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, encapsulated in a LNP, and further comprising or comprising about 5 to 15 mM Tris buffer and about 200 to 400 mM sucrose at a pH of or of about 7.0 to 8.0. In some aspects, one or more of the foregoing elements may be excluded from the liquid RNA-LNP immunogenic composition.

In some aspects, the liquid RNA-LNP immunogenic composition comprises an RNA molecule/polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, and more preferably of or of about 0.06 mg/mL, encapsulated in a LNP, and further comprising or comprising about 10 mM Tris buffer and 300 mM sucrose at a pH of or of about 7.4. In some aspects, one or more of the foregoing elements may be excluded from the liquid RNA-LNP immunogenic composition.

In some aspects, the RNA-LNP immunogenic composition (pre-lyophilized) comprises an RNA molecule/polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, encapsulated in a LNP, and further comprising or comprising about 5 to 15 mM Tris buffer and 200 to 400 mM sucrose at a pH of or of about 7.0 to 8.0, and reconstituted with 0.9% sodium chloride diluent. In some aspects, one or more of the foregoing elements may be excluded from the RNA-LNP immunogenic composition.

In some aspects, the RNA-LNP immunogenic composition (pre-lyophilized) comprises an RNA molecule/polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, and more preferably 0.15 mg/mL, encapsulated in a LNP, and further comprising or comprising about 10 mM Tris buffer and 300 mM sucrose at a pH of or of about 7.4, and reconstituted with 0.9% sodium chloride diluent. In some aspects, one or more of the foregoing elements may be excluded from the RNA-LNP immunogenic composition.

### B. Vaccines

In some aspects, a pharmaceutical composition described herein is an immunogenic composition for inducing an immune response. For example, in some aspects, an immunogenic composition is a vaccine. In some aspects, the compositions described herein include at least one isolated nucleic acid as described herein. In specific aspects, the immunogenic compositions comprise nucleic acids, and the immunogenic compositions are nucleic acid vaccines. In some aspects, the immunogenic compositions comprise RNA (e.g., mRNA), and vaccines are RNA vaccines. In other aspects, the immunogenic compositions comprise DNA, and vaccines are DNA vaccines. Conditions and/or diseases that may be treated with the nucleic acid compositions include, but are not limited to, those caused and/or impacted by infection, cancer, rare diseases, and other diseases or conditions caused by overproduction, underproduction, and/or improper production of protein or nucleic acids.

In some aspects, the composition is substantially free of one or more impurities or contaminants and, for instance, includes nucleic acid or polypeptide molecules that are equal to at least, at most, exactly, or between (inclusive or exclusive) of 90%, 91%, 92%, 93%, 94%, 95%, 96%, 97%, 98%, or 99% pure; at least 98% pure, or at least 99% pure.

The present disclosure includes methods for preventing, treating and/or ameliorating an infection, disease or condition in a subject, including administering to a subject an effective amount of an RNA molecule that includes at least one open reading frame encoding a polypeptide or composition described herein. As such, the disclosure contemplates vaccines for use in both active and passive immunization aspects. In some embodiments, the vaccine is multivalent. Immunogenic compositions, proposed to be suitable for use as a vaccine, may be prepared from RNA molecules encoding polypeptide(s), such as RSV A, RSV B, hMPV A, hMPV B and/or PIV3 F protein mutant and PIV3 HN protein mutant and/or PIV1 F protein mutant. In certain aspects, immunogenic compositions are lyophilized for more ready formulation into a desired vehicle.

The preparation of vaccines that contain nucleic acid as active ingredients is generally well understood in the art, as exemplified by U.S. Pat. Nos. 4,608,251; 4,601,903; 4,599,231; 4,599,230; 4,596,792; and 4,578,770, all of which are hereby incorporated herein by reference. Typically, such vaccines are prepared as injectables either as liquid solutions or suspensions; solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific aspects, vaccines are formulated with a combination of substances, as described in U.S. Pat. Nos. 6,793,923 and 6,733,754, which are incorporated herein by reference. In some aspects, one or more of the foregoing elements may be excluded from a vaccine.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of or of about 0.5% to about 10%. In some aspects, suppositories may be formed from mixtures containing the active ingredient in the range of or of about 1% to about 2%. Oral formulations include such normally employed excipients as, for example, pharmaceutical grades of mannitol, lactose, starch, magnesium stearate, sodium saccharine, cellulose, magnesium carbonate and the like. In some aspects, 1, 2, 3, 4, 5, or more of the foregoing excipients may be excluded from an oral formulation. These compositions take the form of solutions, suspensions, tablets, pills, capsules, sustained release formulations or powders and contain or contain about 10% to about 95% of active ingredient.

The polypeptide-encoding nucleic acid constructs may be formulated into a vaccine as neutral or salt forms. “Pharmaceutically acceptable salt” includes both acid and base addition salts. “Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, toluenesulfonic acid, trifluoroacetic acid, undecylenic acid, and the like. In some aspects, 1, 2, 3, 4, 5, or more of the foregoing inorganic acids may be excluded. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. In some aspects, 1, 2, 3, 4, 5, or more of the foregoing inorganic bases may be excluded. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, 15 ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline, and caffeine. In some aspects, 1, 2, 3, 4, 5, or more of the foregoing organic bases may be excluded.

The polypeptide-encoding nucleic acid constructs and polypeptides, or their pharmaceutically acceptable salts, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (5)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are non-superimposable mirror images of one another. A “tautomer” refers to a proton shift from one atom of a molecule to another atom of the same molecule. The present invention disclosure tautomers of any said compounds.

Compounds described herein that exist in free base or acid form can be converted to their pharmaceutically acceptable salts by treatment with the appropriate inorganic or organic base or acid by methods known to one skilled in the art. Salts of the compounds can be converted to their free base or acid form by standard techniques.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include-C(O)—R″ (where R″ is alkyl, aryl or arylalkyl), p-methoxybenzyl, trityl and the like. Suitable protecting groups for carboxylic acid include alkyl, aryl or arylalkyl esters. As one of skill in the art would appreciate, the protecting group may also be a polymer resin such as a Wang resin, Rink resin or a 2-chlorotrityl-chloride resin. In some aspects, 1, 2, 3, 4, 5, or more of the foregoing protecting groups may be excluded. Protecting groups may be added or removed in accordance with standard 20 techniques, which are known to one skilled in the art (see, e.g., Green, T. W. and P. G. M. Wutz, Protective Groups in Organic Synthesis (1999), 3rd Ed., Wiley) and as described herein.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable, but are typified by an initial administration followed by subsequent inoculations and/or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain aspects, it will be desirable to have one administration of the vaccine. In some aspects, it will be desirable to have multiple administrations of the vaccine, e.g., 2, 3, 4, 5, 6, or more administrations. The vaccinations may be at 1, 2, 3, 4, 5, 6, 7, 8, to 5, 6, 7, 8, 9, 10, 11, or 12 twelve week intervals, including all ranges there between. In some aspects, vaccinations may be at 1, 2, 3, 4, 5, 6, 7, 8, 9, 10, 11, or 12 month intervals, including all ranges there between. Periodic boosters at intervals of 1-5 years may be desirable to maintain protective levels of the antibodies.

i. Carriers

A pharmaceutically acceptable carrier may include the liquid or non-liquid basis of a composition. If a composition is provided in liquid form, the carrier may be water, such as pyrogen-free water; isotonic saline or buffered (aqueous) solutions, e.g. phosphate, citrate buffered solutions. Water or a buffer, such as an aqueous buffer, may be used, containing a sodium salt, a calcium salt, and and/or a potassium salt. The sodium, calcium and/or potassium salts may occur in the form of their halogenides, e.g. chlorides, iodides, or bromides, in the form 20 of their hydroxides, carbonates, hydrogen carbonates, or sulfates, etc. Examples of sodium salts include, but are not limited to, NaCl, NaI, NaBr, Na₂CO₃, NaHCO₃, Na₂SO₄, Na₂HPO₄, Na₂HPO₄·₂H₂O, examples of potassium salts include, but are not limited to, KCl, KI, KBr, K₂CO₃, KHCO₃, K₂SO₄, KH₂PO₄, and examples of calcium salts include, but are not limited to, CaCl₂), CaI₂, CaBr₂, CaCO₃, CaSO₄, Ca(OH)₂. Examples of further carriers may include sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid. Examples of further carriers may include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. In some aspects, 1, 2, 3, 4, 5, or more of the 35 foregoing carriers may be excluded.

ii. Adjuvants

Suitable adjuvants include all acceptable immunostimulatory compounds, such as cytokines, toxins, or synthetic compositions. A number of adjuvants may be used to enhance an antibody response. Adjuvants include, but are not limited to, oil-in-water emulsions, water-in-oil emulsions, mineral salts, polynucleotides, and natural substances. Specific adjuvants that may be used include oil such as MONTANIDE® ISA51, IL1, IL2, IL3, IL4, IL5, IL6, IL7, IL8, IL9, IL10, IL12, alpha-interferon, PTNGg, GM-CSF, GMCSP, BCG, LT-a, aluminum salts, such as aluminum hydroxide or other aluminum compound, MDP compounds, such as thur-MDP and nor-MDP, CGP (MTP-PE), lipid A, monophosphoryl lipid A (MPL), lipopeptides (e.g., Pam3Cys). RIBI, which contains three components extracted from bacteria, MPL, trehalose dimycolate (TDM), and cell wall skeleton (CWS) in a 2% squalene/Tween 80 emulsion. MHC antigens may even be used.

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (CARBOPOL®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (FLUOSOL-DA®) used as a block substitute may also be employed to produce an adjuvant effect.

In some aspects, 1, 2, 3, 4, 5, or more of the foregoing adjuvants may be excluded.

In addition to adjuvants, it may be desirable to co-administer biologic response modifiers (BRM) to enhance immune responses. BRMs have been shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, NJ) and cytokines such as y-interferon, IL-2, or IL-12 or genes encoding proteins involved in immune helper functions, such as B-7.

### C. Combination Therapy

The compositions and related methods of the present disclosure, particularly administration of an RNA molecule encoding a RSV A, RSV B, hMPV A, hMPV B and/or PIV1 and/or PIV3 preF, and/or PIV1 HN and/or PIV3 HN polypeptide, may also be used in combination with the administration of one or more other therapeutic agents. These include, but are not limited to, the administration of traditional therapies, e.g., antiviral therapies such as acyclovir, valacyclovir, and famciclovir, or various combinations of antivirals.

Also included are the administration of one or more therapies to treat one or more symptoms of RSV, hMPV, and/or PIV3 and/or PIV1 infection, including, but not limited to, steroids including corticosteroids, anti-inflammatories including acetaminophen or ibuprofen, pain-relief agents, creams or lotions to relieve itching, cool compresses, or various combinations thereof. In some aspects, 1, 2, 3, 4, 5, or more of the foregoing therapeutic agents may be excluded.

Such combination therapy includes administration of a single pharmaceutical dosage formulation of a composition of the invention and one or more additional active agents, as well as administration of the composition of the invention and each active agent in its own separate pharmaceutical dosage formulation. For example, a composition of the invention and the other active agent can be administered to the patient together in a single dosage composition such as an injection or tablet or capsule, or each agent administered in separate oral dosage formulations. Where separate dosage formulations are used, the compounds of the invention and one or more additional active agents can be administered at essentially the same time, e.g., concurrently, or at separately staggered times, e.g., sequentially; combination therapy is understood to include all these regimens.

In one aspect, it is contemplated that a vaccine and/or therapy is used in conjunction with antiviral treatment. Alternatively, the vaccine and/or therapy may precede or follow treatment with another agent by intervals ranging from minutes to weeks. In aspects where the other agents and/or vaccines are administered separately, one would generally ensure that a significant period of time did not expire between the time of each delivery, such that the agent and immunogenic composition would still be able to exert an advantageously combined effect on the subject. In such aspects, it is contemplated that one may administer both modalities within or within about 12-24 h of each other or within or within about 6-12 h of each other (e.g., within at least, at most, exactly, or between (inclusive or exclusive) any two of 1, 2, 3, 4, 5, 6, 7, 8, 9, 10, 11, 12, 13, 14, 15, 16, 17, 18, 19, 20, 21, 22, 23, or 24 hours). In some situations, it may be desirable to extend the time period for administration significantly, where several days (2, 3, 4, 5, 6, 7, or more) to several weeks (1, 2, 3, 4, 5, 6, 7, 8, or more) lapse between the respective administrations.

Various combinations may be employed, for example antiviral therapy “A” and immunogenic polypeptide given as part of an immune therapy regime “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the immunogenic compositions of the present disclosure to a patient/subject will follow general protocols for the administration of such compounds, taking into account the toxicity, if any, of the RSV, hMPV, PIV1 or PIV3 RNA vaccine composition, or other compositions described herein. It is expected that the treatment cycles would be repeated as necessary. It also is contemplated that various standard therapies, such as hydration, may be applied in combination with the described therapy.

### D. Administration

Administration of the compositions described herein may be carried out via any of the accepted modes of administration of agents for serving similar utilities. In some aspects, a pharmaceutical composition described herein may be administered intranasally, subcutaneously, intradermally or intramuscularly. In specific aspects, the RNA molecules and/or RNA-LNP compositions are administered intramuscularly. In certain aspects, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In one aspect, the pharmaceutical composition is formulated for intramuscular administration. In another aspect, the pharmaceutical composition is formulated for systemic administration, e.g., for intravenous administration. In some aspects, 1, 2, 3, or more of the foregoing administration routes may be excluded.

Pharmaceutical compositions may be formulated into preparations in solid, semi-solid, liquid, lyophilized, frozen, and/or gaseous forms, such as tablets, capsules, powders, granules, ointments, solutions, suspensions, suppositories, injections, inhalants, gels, microspheres, and aerosols. In some aspects, 1, 2, 3, or more of the foregoing preparations may be excluded. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intramuscular, intradermal, intranasal, intrasternal injection, or infusion techniques. Pharmaceutical compositions described herein are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound in aerosol form may hold a plurality of dosage units. The composition to be administered will, in any event, contain a therapeutically and/or prophylactically effective amount of a compound within the scope of this disclosure, or a pharmaceutically acceptable salt thereof, for treatment of a disease or condition of interest in accordance with the teachings described herein.

A pharmaceutical composition within the scope of this disclosure may be in the form of a solid or liquid and may be frozen or lyophilized. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalatory administration. In some aspects, when intended for oral administration, the pharmaceutical composition is in either solid or liquid form, where semi-solid, semi-liquid, suspension, and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present or exclude: binders such as carboxymethylcellulose, ethyl cellulose, microcrystalline cellulose, gum tragacanth, or gelatin; excipients such as starch, lactose, or dextrins; disintegrating agents such as alginic acid, sodium alginate, PRIMOJEL®, corn starch and the like; lubricants such as magnesium stearate or STEROTEX®; glidants such as colloidal silicon dioxide; sweetening agents such as sucrose or saccharin; a flavoring agent such as peppermint, methyl salicylate, or orange flavoring; and a coloring agent. When the pharmaceutical composition is in the form of a capsule, for example, a gelatin capsule, it may contain, in addition to materials of the above type, a liquid carrier such as polyethylene glycol or oil. In some aspects, 1, 2, 3, or more of the foregoing elements may be excluded from a solid composition.

The pharmaceutical composition may be in the form of a liquid, for example, an elixir, syrup, solution, emulsion or suspension. The liquid may be for oral administration or for delivery by injection, as two examples. In some aspects, when intended for oral administration, compositions contain, in addition to the present compounds, one or more of a sweetening agent, preservatives, dye/colorant, and flavor enhancer. In a composition intended to be administered by injection, one or more of a surfactant, preservative, wetting agent, dispersing agent, suspending agent, buffer, stabilizer, and isotonic agent may be included or excluded.

A liquid pharmaceutical composition, whether it be a solution, suspension, or other like form, may include or exclude one or more of the following adjuvants: sterile diluents such as water for injection, saline solution, e.g., physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose; agents to act as cryoprotectants such as sucrose or trehalose. The parenteral preparation may be enclosed in ampoules, disposable syringes, or multiple dose vials made of glass or plastic. In one aspect, physiological saline is the adjuvant. In one aspect, an injectable pharmaceutical composition is sterile. A liquid pharmaceutical composition intended for either parenteral or oral administration should contain an amount of a compound such that a suitable dosage will be obtained.

The pharmaceutical composition may include various materials, which modify the physical form of a solid or liquid dosage unit. For example, the composition may include materials that form a coating shell around the active ingredients. The materials that form the coating shell are typically inert, and may be, for example, sugar, shellac, or other enteric coating agents.

The pharmaceutical composition may include dosage units that can be administered as an aerosol. The term aerosol denote a variety of systems ranging from those of colloidal nature to systems consisting of pressurized packages. Delivery may be by a liquefied or compressed gas or by a suitable pump system that dispenses the active ingredients. Aerosols of compounds may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions may be prepared by methodology well known in the pharmaceutical art. For example, a pharmaceutical composition intended to be administered by injection may be prepared by combining the nucleic acid with sterile distilled water or saline or other carrier so as to form a solution. A surfactant may be added to facilitate the formation of a homogeneous solution or suspension. Surfactants are compounds that non-covalently interact with a compound consistent with the teachings herein so as to facilitate dissolution or homogeneous suspension of the compound in the aqueous delivery system.

The pharmaceutical compositions according to the present disclosure, or their pharmaceutically acceptable salts, are generally applied in a “therapeutically effective amount” or a “prophylactically effective amount” and in “a pharmaceutically acceptable preparation.” The term “pharmaceutically acceptable” refers to the non-toxicity of a material which does not interact with the action of the active component of the pharmaceutical composition. The terms “therapeutically effective amount” and “prophylactically effective amount” refer to the amount which achieves a desired reaction or a desired effect alone or together with further doses. In the case of the treatment of a particular disease, in one aspect, the desired reaction relates to inhibition of the course of the disease. This comprises slowing down the progress of the disease and, in particular, interrupting and/or reversing the progress of the disease. The desired reaction in a treatment of a disease may also be delay of the onset and/or a prevention of the onset of said disease or said condition.

The compositions within the scope of the disclosure are administered in a therapeutically and/or prophylactically effective amount, which will vary depending upon a variety of factors including the activity of the specific therapeutic and/or prophylactic agent employed; the metabolic stability and length of action of the therapeutic and/or prophylactic agent; the individual parameters of the patient, including the age, body weight, general health, gender, and diet of the patient; the mode, time, and/or duration of administration; the rate of excretion; the drug combination; the severity of the particular disorder or condition; and the subject undergoing therapy. In some aspects, 1, 2, 3, 4, 5, or more of the factors may be excluded from determining a therapeutically and/or prophylactically effective amount. Accordingly, the doses administered of the compositions described herein may depend on various of such parameters. In the case that a reaction in a patient is insufficient with an initial dose, higher doses (or effectively higher doses achieved by a different, more localized route of administration) may be used. In some aspects, compositions (e.g., RNA-LNP compositions) may or may not be administered at dosage levels sufficient to deliver at least, at most, exactly, or between (inclusive or exclusive) any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg per kg, of subject body weight per day, one or more times a day, per week, per month, etc. to obtain the desired therapeutic, diagnostic, prophylactic, and/or imaging effect (see, e.g., the range of unit doses described in International Publication No. WO2013/078199, herein incorporated by reference in its entirety).

In some aspects, compositions (e.g., RNA-LNP compositions) may or may not be administered at a total dose of or at dosage levels sufficient to deliver a total dose of at least, at most, exactly, or between (inclusive or exclusive) any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg per day, one or more times a day, per week, per month, etc. to obtain the desired therapeutic, diagnostic, prophylactic, and/or imaging effect.

In specific aspects, compositions (e.g., RNA-LNP compositions) may or may not be administered at a total dose of or at dosage levels sufficient to deliver a total dose of at least, at most, exactly, or between (inclusive or exclusive) any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL RNA encapsulated in LNP.

In exemplary aspects, compositions (e.g., RNA-LNP compositions) may or may not be administered at dose levels of at least, at most, exactly, or between (inclusive or exclusive) any two of 0.01, 0.15, 0.30, 0.45, 0.60, 0.75, or 0.90 mg/mL RNA encapsulated in LNP. In exemplary aspects, compositions (e.g., RNA-LNP compositions) may or may not be administered at dose levels of at least, at most, exactly, or between (inclusive or exclusive) any two of 0.01, 0.15, 0.30, 0.45, 0.60, 0.75, or 0.90 mg or 10, 15, 30, 45, 60, 75, 90, or 100 μg RNA encapsulated in LNP.

In specific aspects, compositions (e.g., RNA-LNP compositions) may or may not be administered at a total dose of or at dosage levels sufficient to deliver a total dose of at least, at most, exactly, or between (inclusive or exclusive) any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL RNA encapsulated in LNP.

In exemplary aspects, compositions (e.g., RNA-LNP compositions) may or may not be administered at dose levels of at least, at most, exactly, or between (inclusive or exclusive) any two of 1, 15, 30, 45, 60, 75, 90, 100 or higher μg/mL RNA encapsulated in LNP. In exemplary aspects, compositions (e.g., RNA-LNP compositions) may or may not be administered at dose levels of at least, at most, exactly, or between (inclusive or exclusive) any two of 1, 15, 30, 45, 60, 75, 90, 100 or higher μg RNA encapsulated in LNP.

The desired dosage may be delivered every two weeks, every three weeks, every four weeks, every 2 months, every three months, every 6 months, every year, etc. In certain aspects, the desired dosage may be delivered using a single-dose administration. In certain aspects, the desired dosage may be delivered using multiple administrations (e.g., two, three, four, five, six, seven, eight, nine, ten, eleven, twelve, thirteen, fourteen, or more administrations). When multiple administrations are employed, split dosing regimens may be used. The time of administration between the initial administration of the composition and a subsequent administration of the composition may be, but is not limited to, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In some aspects, compositions (e.g., RNA-LNP compositions) may be administered in a single dose. In some aspects, compositions (e.g., RNA-LNP compositions) may be administered twice (e.g., Day 0 and on or about Day 7, Day 0 and on or about Day 14, Day 0 and on or about Day 21, Day 0 and on or about Day 28, Day 0 and on or about Day 60, Day 0 and on or about Day 90, Day 0 and on or about Day 120, Day 0 and on or about Day 150, Day 0 and on or about Day 180, Day 0 and on or about 1 month later, Day 0 and on or about 2 months later, Day 0 and on or about 3 months later, Day 0 and on or about 6 months later, Day 0 and on or about 9 months later, Day 0 and on or about 12 months later, Day 0 and on or about 18 months later, Day 0 and on or about 2 years later, Day 0 and on or about 5 years later, or Day 0 and on or about 10 years later), with each administration at a total dose of or at dosage levels sufficient to deliver a total dose of at least, at most, exactly, or between (inclusive or exclusive) any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg RNA encapsulated in LNP. Higher and lower dosages and frequency of administration are encompassed by the present disclosure. For example, compositions (e.g., RNA-LNP compositions) may be administered three or four times.

Periodic boosters at intervals of 1, 2, 3, 4 or 5 years or more may be desirable to maintain protective levels of the antibodies. As used herein, the term “booster” refers to an extra administration of a composition (e.g., a RNA-LNP composition). A booster may be given after an earlier administration of the composition.

In some aspects, the compositions (e.g., RNA-LNP compositions) are or are not administered to a subject as a single dose of at least, at most, exactly, or between (inclusive or exclusive) any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg of RNA encapsulated in LNP. In some aspects, the compositions (e.g., RNA-LNP compositions) are or are not administered the subject as a single dose of at least, at most, exactly, or between (inclusive or exclusive) any two of 1 μg, 15 μg, 30 μg, 45 μg, 60 μg, 75 μg, 90 μg, 100 μg or higher of RNA encapsulated in LNP.

In some aspects, the compositions (e.g., RNA-LNP compositions) are or are not administered to a subject as two doses of at least, at most, exactly, or between (inclusive or exclusive) any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg of RNA encapsulated in LNP. In some aspects, the compositions (e.g., RNA-LNP compositions) are or are not administered the subject as two doses of at least, at most, exactly, or between (inclusive or exclusive) any two of 1 μg, 15 μg, 30 μg, 45 μg, 60 μg, 75 μg, 90 μg, 100 μg or higher of RNA encapsulated in LNP.

In specific aspects, compositions (e.g., RNA-LNP compositions) may or may not be administered twice (e.g., Day 0 and Day 28, Day 0 and Day 60, Day 0 and Day 180, Day 0 and 2 months later, Day 0 and 6 months later, Day 0 and one year later, etc.), with each administration at a total dose of or at dosage levels sufficient to deliver a total dose of at least, at most, exactly, or between (inclusive or exclusive) any two of 1 μg, 15 μg, 30 μg, 45 μg, 60 μg, 75 μg, 90 μg, 100 μg or higher RNA encapsulated in LNP.

### IX. Methods of Use

Provided herein are compositions (e.g., pharmaceutical compositions comprising RNA molecules and/or RNA-LNPs), methods, kits and reagents for prevention and/or treatment of in humans and other mammals.

RNA compositions (e.g., RNA-LNP compositions) may be used as therapeutic and/or prophylactic agents. They may be used in medicine to prevent and/or treat infectious disease. In exemplary aspects, the RNA compositions of this invention are used to provide prophylactic protection from acute lower respiratory infection (ALRI) or lower respiratory tract disease (LRTD) of any genotype, strain, or isolate. It is envisioned that there may be situations where persons are at risk for infection with more than one strain of RSV, hMPV, PIV1 or PIV3. RNA compositions (e.g., RNA-LNP compositions) are particularly amenable to combination vaccination approaches due to a number of factors including, but not limited to, speed of manufacture, ability to rapidly tailor vaccines to accommodate perceived geographical threat, and the like. Moreover, because the RNA compositions (e.g., RNA-LNP compositions) utilize the human body to produce the antigenic protein, the RNA compositions (e.g., RNA-LNP compositions) are amenable to the production of larger, more complex antigenic proteins, allowing for proper folding, surface expression, antigen presentation, etc. in the human subject. To protect against more than one strain of RSV, a combination RSV RNA composition can be administered that includes RNA encoding at least one antigenic polypeptide protein (or antigenic portion thereof) of a first RSV antigen and further includes RNA encoding at least one antigenic polypeptide protein (or antigenic portion thereof) of a second antigen, for example RSV A and RSV B. To protect against more than one respiratory virus, a combination RNA composition can be administered that includes RNA encoding at least one antigenic polypeptide protein (or antigenic portion thereof) of a first antigen and further includes RNA encoding at least one antigenic polypeptide protein (or antigenic portion thereof) of a second antigen, for example RSV A, RSV B, hMPV A, hMPV B, and/or PIV1 and PIV3. The vaccines of the present disclosure may be used to prevent RSV, hMPV, PIV1 and/or PIV3 infection-associated illness (including pneumonia and bronchitis) and may be particularly useful for prevention and/or treatment of immunocompromised and elderly patients to prevent or to reduce the severity and/or duration of viral respiratory infection.

In some aspects, the RNA compositions (e.g., RNA-LNP compositions) of the disclosure are administered to a subject (e.g., a mammalian subject, such as a human subject), and the RNA polynucleotides are translated in vivo to produce an antigenic polypeptide. The RNA compositions (e.g., RNA-LNP compositions) may be induced for translation of a polypeptide (e.g., antigen or immunogen) in a cell, tissue or organism. In exemplary embodiments, such translation occurs in vivo, although there can be envisioned embodiments where such translation occurs ex vivo, in culture or in vitro. In exemplary embodiments, the cell, tissue or organism is contacted with an effective amount of a RNA composition (e.g., a RNA-LNP composition) including an RNA molecule having at least one a translatable region encoding an antigenic polypeptide (e.g., a RSV, hMPV, PIV1 and/or PIV3 antigen).

In some aspects, the RNA compositions of the disclosure may be used to prime immune effector cells, for example, to activate peripheral blood mononuclear cells (PBMCs) ex vivo, which are then infused (re-infused) into a subject.

In some aspects, after administration of a RNA molecule described herein, e.g., formulated as RNA-LNPs, at least a portion of the RNA is delivered to a target cell.

In some aspects, at least a portion of the RNA is delivered to the cytosol of the target cell. In some aspects, the RNA is translated by the target cell to produce the polypeptide or protein it encodes. In some aspects, the target cell is a spleen cell. In some aspects, the target cell is an antigen presenting cell such as a professional antigen presenting cell in the spleen. In some aspects, the target cell is a dendritic cell and/or macrophage. RNA molecules such as RNA-LNPs described herein may be used for delivering RNA to such target cell. Accordingly, the present disclosure also relates to a method for delivering RNA to a target cell in a subject comprising the administration of the RNA-particles described herein to the subject.

In some aspects, the RNA is delivered to the cytosol of the target cell. In some aspects, the RNA is translated by the target cell to produce the polypeptide or protein encoded by the RNA. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, may be referred to as encoding the protein or other product of that gene or cDNA.

In some aspects, nucleic acid compositions described herein, e.g., compositions comprising a RNA-LNP, are characterized by (e.g., when administered to a subject) an induced and/or boosted immune response as a function of antigen production in the cell. Increased antigen production may be demonstrated by, e.g., increased cell transfection (the percentage of cells transfected with the RNA vaccine), increased protein translation from the polynucleotide, decreased nucleic acid degradation (as demonstrated, for example, by increased duration of protein translation from a modified polynucleotide), and/or altered antigen specific immune response of the host cell.

In some aspects, the disclosure relates to a method of inducing an immune response against RSV, hMPV, PIV1 and/or PIV3 in a subject. The method includes administering to the subject an effective amount of an RNA molecule, RNA-LNP and/or composition as described herein to produce an immune response against RSV, hMPV, PIV1 and/or PIV3.

In another aspect, the disclosure relates to a method of vaccinating a subject. The method includes administering to the subject in need thereof an effective amount of an RNA molecule, RNA-LNP and/or composition described herein.

In another aspect, the disclosure relates to a method of treating and/or preventing an infectious disease. The method includes administering to the subject an effective amount of an RNA molecule RNA-LNP and/or composition as described herein.

In another aspect, the disclosure relates to a method of treating and/or preventing and/or reducing the severity of a infection and/or illness caused by RSV, hMPV, PIV1 and/or PIV3. The method includes administering to the subject an effective amount of an RNA molecule, RNA-LNP and/or composition as described herein.

In another aspect, the disclosure relates to a method of treating and/or preventing and/or reducing the severity of an infectious disease in a subject by, for example, inducing an immune response to an infectious disease in the subject. In some aspects, the method includes administering a priming composition that includes an effective amount of an RNA molecule, RNA-LNP and/or composition described herein, and administering a booster composition including an effective amount of an RNA molecule, RNA-LNP and/or composition. In some aspects, the composition elicits an immune response including an antibody response. In some aspects, the composition elicits an immune response including a T cell response and/or a B cell response. In some aspects, an immune response comprises a T cell response and a B cell response. In some aspects, the composition elicits a neutralizing immune response. A neutralizing immune response is an immune response that is a neutralizing antibody response and/or an effective neutralizing T cell response. In some embodiments a neutralizing antibody response produces a level of antibodies that meet or exceed a seroprotection threshold. In some aspects, the composition elicits an effective T cell response. An effective T cell response is a response which produces a baseline level of infectious disease-activated and/or infectious disease-specific T cells including CD8+ and CD4+ T helper type 1 cells. In some aspects, the effective T cells comprises a high proportion of CD8+ T cells and/or CD4+ T cells, relative to a baseline level (in a naive subject). In some embodiments these T cells are differentiated towards an early-differentiated memory phenotype with co-expression of CD27 and CD28.

In another aspect, the disclosure relates to a method of treating and/or preventing and/or reducing the severity of an infection and/or illness caused by RSV, hMPV, PIV1 and/or PIV3 in a subject by, for example, inducing an immune response to RSV A, RSV B, hMPV A, hMPV B, PIV1 and/or PIV3 in the subject. In some aspects, the method includes administering a priming composition that includes an effective amount of an RNA molecule, RNA-LNP and/or composition described herein, and administering a booster composition including an effective amount of an RNA molecule RNA-LNP and/or composition as described herein. In some aspects, the composition elicits an immune response including an antibody response. In some aspects, the composition elicits an immune response including a T cell response and/or a B cell response. In some aspects, an immune response comprises a T cell response and a B cell response. In some aspects, the composition elicits a neutralizing immune response. A neutralizing immune response is an immune response that is a neutralizing antibody response and/or an effective neutralizing T cell response. In some embodiments a neutralizing antibody response produces a level of antibodies that meet or exceed a seroprotection threshold. In some aspects, the composition elicits an effective T cell response. An effective T cell response is a response which produces a baseline level of infectious disease-activated and/or infectious disease-specific T cells including CD8+ and CD4+ T helper type 1 cells. In some aspects, the effective T cells comprises a high proportion of CD8+ T cells and/or CD4+ T cells, relative to a baseline level (in a naive subject). In some embodiments these T cells are differentiated towards an early-differentiated memory phenotype with co-expression of CD27 and CD28.

The methods disclosed herein may involve administering to the subject a RNA-LNP composition comprising at least one RNA molecule having an open reading frame encoding at least one antigenic polypeptide, thereby inducing in the subject an immune response specific to the antigenic polypeptide, wherein anti-antigenic polypeptide antibody titer in the subject is increased following vaccination relative to anti-antigenic polypeptide antibody titer in a subject vaccinated with a prophylactically effective dose (e.g., a therapeutically effective dose that prevents infection with the virus at a clinically acceptable level) of a traditional vaccine. An “anti-antigenic polypeptide antibody” is a serum antibody the binds specifically to the antigenic polypeptide. In some aspects, the anti-antigenic polypeptide antibody titer in the subject is or is not increased at least, at most, between (inclusive or exclusive) any two of, or exactly 1, 2, 3, 4, 5, 6, 7, 8, 9, or 10 log following administration of the RNA-LNP composition relative to anti-antigenic polypeptide antibody titer in a subject administered a prophylactically effective dose of a traditional composition against the virus (e.g., the standard of care dose of a recombinant or purified protein vaccine, a live attenuated or inactivated antigen vaccine, or a VLP vaccine). In some aspects, the anti-antigenic polypeptide antibody titer in the subject is or is not increased at least, at most, between (inclusive or exclusive) any two of, or exactly 1-, 2-, 3-, 4-, 5-, 6-, 7-, 8-, 9-, 10-, 100-, or 1000-fold following administration of the RNA-LNP composition relative to anti-antigenic polypeptide antibody titer in a subject administered a prophylactically effective dose of a traditional composition against the antigen (e.g., the standard of care dose of a recombinant or purified protein vaccine, a live attenuated or inactivated antigen vaccine, or a VLP vaccine).

In some aspects, an effective amount of a RNA-LNP composition comprising at least one RNA molecule having an open reading frame encoding at least one antigenic polypeptide results in a 2-fold to 200-fold (e.g., at least, at most, exactly, or between (inclusive or exclusive) any two of a 2-, 3-, 4-, 5-, 6-, 7-, 8-, 9-, 10-, 20-, 30-, 40-, 50-, 60-, 70-, 80-, 90-, 100-, 110-, 120-, 130-, 140-, 150-, 160-, 170-, 180-, 190-, or 200-fold) increase in serum neutralizing antibodies against the virus, relative to a traditional composition against the virus (e.g., the standard of care dose of a recombinant or purified protein vaccine, a live attenuated or inactivated antigen vaccine, or a VLP vaccine).

In some aspects, an effective amount of a RNA-LNP composition comprising at least one RNA molecule having an open reading frame encoding at least one antigenic polypeptide is a dose equivalent to an at least 2-fold reduction in a standard of care dose of a traditional composition against the virus. For example, an effective amount of a RNA-LNP composition may or may not be a dose equivalent to a 2-, 3-, 4-, 5-, 6-, 7-, 8-, 9-, 10-, 20-, 50-, 100-, 250-, 500-, or 1000-fold reduction in a standard of care dose of a traditional composition against the virus. In some embodiments, the anti-antigenic polypeptide antibody titer produced in a subject administered an effective amount of a RNA-LNP composition is equivalent to an anti-antigenic polypeptide antibody titer produced in a control subject administered the standard of care dose of a traditional composition against the virus. In some embodiments, an effective amount of a RNA-LNP composition is or is not a dose equivalent to a 2-fold to 1000-fold reduction (e.g., at least, at most, exactly, or between (inclusive or exclusive) any two of a 2-, 3-, 4-, 5-, 6-, 7-, 8-, 9-, 10-, 20-, 30-, 40-, 50-, 60-, 70-, 80-, 90-, 100-, 110-, 120-, 130-, 140-, 150-, 160-, 170-, 1280-, 190-, 200-, 210-, 220-, 230-, 240-, 250-, 260-, 270-, 280-, 290-, 300-, 310-, 320-, 330-, 340-, 350-, 360-, 370-, 380-, 390-, 400-, 410-, 420-, 430-, 440-, 450-, 4360-, 470-, 480-, 490-, 500-, 510-, 520-, 530-, 540-, 550-, 560-, 5760-, 580-, 590-, 600-, 610-, 620-, 630-, 640-, 650-, 660-, 670-, 680-, 690-, 700-, 710-, 720-, 730-, 740-, 750-, 760-, 770-, 780-, 790-, 800-, 810-, 820-, 830-, 840-, 850-, 860-, 870-, 880-, 890-, 900-, 910-, 920-, 930-, 940-, 950-, 960-, 970-, 980-, 990-, or 1000-fold reduction) in the standard of care dose of a traditional composition against the virus, wherein the anti-antigenic polypeptide antibody titer produced in the subject is equivalent to an anti-antigenic polypeptide antibody titer produced in a control subject administered the standard of care dose of a traditional composition against the virus. In some aspects, an anti-antigenic polypeptide antibody titer produced in the subject is equivalent to an anti-antigenic polypeptide antibody titer produced in a control subject administered the standard of care dose of a traditional composition against virus.

A traditional composition against a virus, as used herein, refers to a composition other than the RNA molecules, RNA-LNPs and/or compositions described herein. For instance, a traditional composition includes, but is not limited, to live microorganism vaccines, killed microorganism vaccines, attenuated vaccines, subunit vaccines, protein antigen vaccines containing recombinant protein produced in a heterologous expression system or purified from large amounts of the pathogenic organism, DNA vaccines, virus-like particle (VLP) vaccines containing viral capsid proteins (e.g., pre- and/or post-fusion F proteins) but lacking viral genome, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA). A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a traditional composition against a virus that a physician/clinician or other medical professional would administer to a subject to treat and/or prevent viral infection, or a viral-related condition, while following the standard of care guideline for treating and/or preventing viral infection, or a viral-related condition.

In some aspects, an RNA molecule, RNA-LNP and/or composition described herein (e.g., a RNA-LNP composition comprising at least one RNA molecule having an open reading frame encoding at least one antigenic polypeptide) produces prophylactically- and/or therapeutically-efficacious levels, concentrations and/or titers of antigen-specific antibodies in the blood or serum of a subject. As defined herein, the term antibody titer refers to the amount of antigen-specific antibody produces in a subject, e.g., a human subject. In exemplary embodiments, antibody titer is expressed as the inverse of the greatest dilution (in a serial dilution) that still gives a positive result. In exemplary aspects, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In exemplary aspects, an efficacious RNA molecule, RNA-LNP and/or composition described herein (e.g., a RNA-LNP composition comprising at least one RNA molecule having an open reading frame encoding at least one antigenic polypeptide) produces an antibody titer of greater than 1:10, greater that 1:100, greater than 1:400, greater than 1:1000, greater than 1:2000, greater than 1:3000, greater than 1:4000, greater than 1:5000, greater than 1:6000, greater than 1:7500, or greater than 1:10000. In exemplary aspects, the antibody titer is produced or reached by 10 days following vaccination, by 20 days following vaccination, by 30 days following vaccination, by 40 days following vaccination, or by 50 or more days following vaccination. In exemplary aspects, the titer is produced or reached following a single dose of vaccine administered to the subject. In other aspects, the titer is produced or reached following multiple doses, e.g., following a first and a second dose (e.g., a booster dose).

The methods disclosed herein may involve administering to the subject a RNA-LNP composition comprising at least one RNA molecule having an open reading frame encoding at least one antigenic polypeptide, thereby inducing in the subject an immune response specific to an antigenic polypeptide, wherein the immune response in the subject is equivalent to an immune response in a subject administered with a traditional composition against the virus that is or is not at least, at most, in between (inclusive or exclusive) any two of, or exactly 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 times the dosage level relative to the RNA composition.

In some aspects, the RNA molecule, RNA-LNP and/or composition is used as a vaccine. In some aspects, the RNA molecule, RNA-LNP and/or composition may be used in various therapeutic or prophylactic methods for preventing, treating or ameliorating of acute lower respiratory infection (ALRI) or lower respiratory tract disease (LRTD), or a disorder related to respiratory illness, including pneumonia and bronchitis.

In some aspects, the RNA molecule, RNA-LNP and/or composition may be used in various therapeutic or prophylactic methods for preventing, treating or ameliorating of acute lower respiratory infection (ALRI) or lower respiratory tract disease (LRTD), including pneumonia and bronchitis.

RNA compositions may be administered prophylactically to healthy subjects. In some aspects, the subject is immunocompetent. In some aspects, the subject is immunocompromised.

In some aspects, the RNA molecule, RNA-LNP and/or composition is administered in a single dose. In some aspects, a second, third or fourth dose may be given. In some aspects, the RNA molecule, RNA-LNP and/or composition is administered in multiple doses.

In some aspects, the RNA molecule, RNA-LNP and/or composition is administered intramuscularly (IM) or intradermally (ID).

The present disclosure further provides a kit comprising the RNA molecule, RNA-LNP, and/or composition.

In some aspects, the RNA molecule, RNA-LNP and/or composition described herein is administered to a subject that is less than about 1 years old, or about 1 years old to about 10 years old, or about 10 years old to about 20 years old, or about 20 years old to about 50 years old, or about 60 years old to about 70 years old, or older.

In some aspects the subject is at least, at most, exactly, or between any two of less than 1 year of age, greater than 1 year of age, greater than 5 years of age, greater than 10 years of age, greater than 20 years of age, greater than 30 years of age, greater than 40 years of age, greater than 50 years of age, greater than 60 years of age, greater than 70 years of age, or older. In some aspects, the subject is greater than 50 years of age.

In some aspects the subject is at least, at most, exactly, or between (inclusive or exclusive) any two of about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older. In some aspects, the subject may be about 50 years of age or older.

In some embodiments, the composition of the present invention comprises a nucleic acid encoding a respiratory syncytial virus subtype A (RSV A) F protein mutant. In some embodiments, the composition of the present invention further comprises a nucleic acid encoding a respiratory syncytial virus subtype B (RSV B) F protein mutant. In further embodiments, the composition of the present invention further comprises nucleic acids encoding a RSV A F protein mutant and a RSV B F protein mutant. In some embodiments, the composition of the present invention further comprises a nucleic acid encoding a hMPV A F protein mutant. In some embodiments, the composition of the present invention further comprises a nucleic acid encoding a hMPV B F protein mutant. In further embodiments, the composition of the present invention further comprises nucleic acids encoding a hMPV A F protein mutant and a hMPV B F protein mutant. hMPV F protein mutants are described in any of PCT Publication Nos. WO16103238, WO20234300, WO21222639, WO22076669, WO22214678, WO23102373, WO23110618, WO23217988, and WO23102388, the full disclosures of which are herein incorporated by reference in their entirety for all purposes.

In some embodiments, the composition of the present invention further comprises a nucleic acid encoding a PIV1 F protein mutant. In some embodiments, the composition of the present invention further comprises a nucleic acid encoding a PIV3 F protein mutant. In further embodiments, the composition of the present invention further comprises nucleic acids encoding a PIV1 F protein mutant and a PIV3 F protein mutant. PIV1 and PIV3 F protein mutants are described in any of PCT Publication Nos. WO2018081289, and WO2022207839, the full disclosures of which are herein incorporated by reference in their entirety for all purposes.

The vaccines provided by the present disclosure may be used together with one or more other vaccines. For example, in adults they may be used together with an influenza vaccine, Prevnar, tetanus vaccine, diphtheria vaccine, COVID19 vaccine and pertussis vaccine. For pediatric use, vaccines provided by the present disclosure may be used with any other vaccine indicated for pediatric patients.

### X. Clinical Studies

The RNA-LNP vaccines of the present disclosure comprise nucleoside-modified mRNA encoding RSV A, RSV B, hMPV A, hMPV B and/or PIV3 and/or PIV1 prefusion F (preF) polypeptide and/or PIV3 and/or PIV1 HN polypeptide (modified RNA; modRNA). The RNA-LNP vaccines may comprise RNA comprising a single-stranded, 5′-capped and polyadenylated modified RNA that is translated after entering the cell. The RNA comprises an open reading frame (ORF) that encodes variations of the polypeptide. Further, as described herein, the RNA may comprise structural elements, such as untranslated regions (UTRs), optimized for high efficacy of the RNA. The RNA-LNPs may comprise RNA as provided in Tables 27, 29, 31, 33, 35 or 37 of Example 1 disclosed herein. The RNA-LNPs may comprise RNA as provided in Tables 3, 6, 9, 12, 15 or 18 disclosed herein. The RNA may also comprise a substitution of N1-methyl-pseudouridine for uridine to decrease recognition of the vaccine RNA by innate immune sensors, such as toll-like receptors (TLRs) 7 and 8, resulting in decreased innate immune activation and increased protein translation.

The RNA molecules described herein are formulated/encapsulated into lipid nanoparticles (LNPs) to enable delivery of the RNA into host cells after, e.g., intramuscular (IM), intradermal (ID), or intranasal (IN) injection. The LNP formulation may comprise two functional lipids, ALC-0315 and ALC-0159, and two structural lipids, DSPC (1,2-distearoyl-sn-glycero-3-phosphocholine) and cholesterol. In some aspects, 1, 2, 3, or more of the forgoing lipids may be excluded from the LNP formulation. The potency of RNA vaccines is optimized by LNP encapsulation, which protects the RNA from degradation by extracellular RNases and facilitates delivery in the cell. After IM injection of RNA-LNP vaccines, the LNPs are taken up by the cells, and the RNA is released into the cytosol. In the cytosol, the RNA is translated, and the encoded viral antigen is produced.

The Examples herein demonstrate the RNA-LNP vaccines of the present disclosure are immunogenic in mice and induce both humoral and cell mediated immune responses in mice.

Clinical studies of the present disclosure evaluate the safety, tolerability, and immunogenicity of RNA-LNP vaccines against RSV, hMPV and/or PIV1 and PIV3 or any combination thereof. For example, the RNA-LNPs vaccines may be indicated for active immunization for the prevention of acute lower respiratory infection (ALRI) or lower respiratory tract disease (LRTD), including pneumonia and bronchitis caused by RSV, hMPV, PIV1 and/or PIV3 for adults (e.g., ≥45, ≥50, ≥55, ≥60, ≥70 . . . etc. years of age or 50 through 69 years of age). In another aspect, the RNA-LNPs vaccines may be indicated for active immunization for the prevention of lower respiratory tract disease (LRTD), including pneumonia and bronchitis, caused by RSV, hMPV, PIV1 and/or PIV3 for adults (e.g., ≥45, ≥50, ≥55, ≥60, ≥70 . . . etc. years of age or 50 through 69 years of age). RNA-LNP vaccines may be administered in different dose level(s), dose formulation, number of doses and dosing schedules, as described herein, including but not limited to:
As a single-dose schedule or a two-dose schedule (e.g., Day 0 and on or about 2 months after or Day 0 and on or about 6 months after)
At different dose levels (e.g., of or of about 15 μg, 30 μg, 45 μg, 60 μg, 90 μg, 100 μg or higher per administration)
At different formulations (non-lyophilized and/or lyophilized)

The RNA-LNPs may be presented as a liquid or lyophilized formulation. Administration of the RNA-LNP vaccines may or may not be dosed in the range of or of about 15 μg, 30 μg, 45 μg, 60 μg, 90 μg, 100 μg or higher per dose with an injection volume of or of about 0.25 to 1 mL (e.g., of or of about 0.25, 0.5, 1 mL). Dilution with sterile 0.9% sodium chloride (normal saline) may be required.

The objectives of RNA-LNP clinical studies may include, but are not limited to:
To describe the safety and tolerability profile of RNA-LNP vaccines administered at selected dose levels and schedules in participants.
To describe the immune responses elicited by commercially-available vaccines against viral vaccines administered at selected dose levels and schedules in participants.

In some aspects, the efficacy (or effectiveness) against RSV, hMPV and/or PIV1 and PIV3 of the RNA molecules encoding RSV A, RSV B, hMPV A, hMPV B, and/or PIV1 and PIV3 polypeptides, RNA-LNPs and compositions thereof disclosed herein is or is not greater than 50% (e.g., at least, at most, exactly, or between any two of 50%, 60%, 70%, 80%, 90%, or more). Vaccine efficacy may be assessed using standard analyses (see, e.g., Weinberg et al, J Infect Dis. 2010 Jun. 1; 201 (11): 1607-10). For example, vaccine efficacy may be measured by double-blind, randomized, clinical controlled trials such as those described herein. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$Efficacy = ( ARU - ARV ) / ARU \times 100 ; and$$

$$Efficacy = ( 1 - RR ) \times 100.$$

Likewise, vaccine effectiveness may be assessed using standard analyses (see, e.g., Weinberg et al., J Infect Dis. 2010 Jun. 1; 201 (11): 1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the real-world outcomes of hospitalizations, ambulatory visits, and/or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - OR ) \times 100.$$

In some aspects, efficacy of the RNA-LNPs and compositions thereof is at least 60% relative to unvaccinated control subjects. For example, efficacy may be at least, at most, exactly, or between (inclusive or exclusive) any two of 65%, 70%, 75%, 80%, 85%, 95%, 98%, or 100% relative to unvaccinated control subjects.

### EXAMPLES

Below are examples of specific aspects for carrying out the present disclosure. The following examples are included to demonstrate aspects of the disclosure. The examples are offered for illustrative purposes only and are not intended to limit the scope of the present disclosure in any way. It should be appreciated by those of skill in the art that the techniques disclosed in the examples which follow represent techniques discovered by the inventor to function well in the practice of the disclosure. However, those of skill in the art should, in light of the present disclosure, appreciate that many changes may be made in the specific aspects which are disclosed and still obtain a like or similar result without departing from the spirit and scope of the disclosure. Efforts have been made to ensure accuracy with respect to numbers used (e.g., amounts, temperatures, etc.), but some experimental error and deviation should, of course, be allowed for.

### Example 1: Nucleoside-Modified Messenger RNA (modRNA) Production

DNA sequences encoding full-length antigens described herein or fragments thereof were prepared using standard molecular biology techniques and utilized for in vitro transcription reactions to generate RNA. In vitro transcription of RNA is known in the art and is described herein. DNA templates were cloned into a plasmid vector with backbone sequence elements (T7 promoter, 5′ and 3′ UTR, poly-A tail) for improved RNA stability and translational efficiency. The DNA was purified, spectrophotometrically quantified and in vitro-transcribed by T7 RNA polymerase in the presence of a trinucleotide cap1 analogue ((m₂^{7,3′-O})Gppp(m₁^{2′-O})ApG) (TriLink BioTechnologies) and with N1-methylpseudouridine (m1Ψ) instead of uridine (modified RNA (modRNA)).

Alternatively, plasmid DNA was linearized immediately following the 3′ end poly-A tail of the modRNA sequence by restriction enzyme digestion and purified by phenol-chloroform. Linearized DNA templates were transcribed into RNA using T7 polymerase, native and N1-methyl pseudouridine (m1ψ) ribonucleotides, and co-transcriptionally capped using Clean Cap reagent (Trilink). RNA in-vitro transcription reaction was stopped by addition of Turbo DNAse (Thermo Fisher) to digest template DNA and modRNA was purified by LiCl precipitation. Plasmids were amplified in Escherichia coli and purified using Qiagen Plasmid Maxi kits (Qiagen).

The exemplified modRNA sequences described herein encode full-length protein mutants comprising the ectodomain, transmembrane domain and cytoplasmic domain of the protein (unless specifically denoted that such domain(s) is/are deleted in a mutant set forth herein). In addition, the exemplified modRNA sequences are Homo sapiens-codon optimized.

Tables 26, 28, 30, and 34 describe the RSV, hMPV and/or PIV1 and/or PIV3 F, Table 32 describes PIV1 HN and Table 36 describes PIV3 HN protein antigens of the present disclosure. Tables 27, 29, 31, and 35 describe the RNA constructs that encode for RSV, hMPV and/or PIV1 and PIV3 F proteins, Table 33 describes the RNA constructs that encode for PIV1 HN proteins and Table 37 describes the RNA constructs that encode for PIV3 HN proteins of the present disclosure, and corresponding sequences, comprising a 5′ UTR, an open reading frame encoding the protein antigens of the present disclosure, a 3′ UTR and a poly-A tail.

1. RSV preF modRNA Constructs

RNA constructs generated herein (Table 27) encode RSV F protein wild-type (WT) and RSV F protein variants/mutants (e.g. RSV pre-fusion F protein). Table 26 describes WT F (WT F) and variant RSV preF mutants.

TABLE 26
RSV F proteins and description

| RSV F Protein | |
|---|---|
| Sequence | RSV F Protein Description |
| SEQ ID NO: 1 | Full Length F0 of Native RSV A2 (GenBank GI: 138251; Swiss Prot |
| | P03420) (574 aa) |
| SEQ ID NO: 2 | Full Length F0 of Native RSV B (18537 strain; GenBank GI: 138250; |
| | Swiss Prot P13843) (574 aa) |
| RSV A WT F | Full length WT RSV subtype A, F protein (574 aa) |
| SEQ ID NO: 3 | |
| RSV F 847A | Full length RSV subtype A 847 mutant F protein with substitutions |
| SEQ ID NO: 4 | A103C, I148C, S190I and D486S (574 aa) |
| RSV B WT F | Full length WT RSV subtype B, F protein (574 aa) |
| SEQ ID NO: 5 | |
| RSV F 847B | Full length RSV subtype B 847 mutant F protein with substitutions |
| SEQ ID NO: 6 | A103C, I148C, S1901 and D486S (574 aa) |
| RSV F 851A | Full length RSV subtype A 851 mutant F protein with substitutions |
| SEQ ID NO: 621 | T54H, A103C, I148C, S190I, V296I and D486S (574 aa) |
| RSV F 851B | Full length RSV subtype B 851 mutant F protein with substitutions |
| SEQ ID NO: 622 | T54H, A103C, I148C, S190I, V296I and D486S (574 aa) |
| RSV F 852A | Full length RSV subtype A 852 mutant F protein with substitutions |
| SEQ ID NO: 623 | T54H S55C, L188C and D486S (574 aa) |
| RSV F 852B | Full length RSV subtype B 852 mutant F protein with substitutions |
| SEQ ID NO: 624 | T54H S55C, L188C and D486S (574 aa) |
| RSV F 847A Ecto- | RSV subtype A 847 mutant F protein with substitutions A103C, I148C, |
| Foldon | S190I and D486S; is truncated after position 513 (TM and CT removed) |
| SEQ ID NO: 625 | and T4 Fibritin Foldon domain (27 amino acids; SEQ ID NO: 45) is |
| | added to the 847A mutant ectodomain via an SAIG linker to form a 574 |
| | aa mutant polypeptide |
| RSV F 847B Ecto- | RSV subtype B 847 mutant F protein with substitutions A103C, I148C, |
| Foldon | S190I and D486S; is truncated after position 513 (TM and CT removed) |
| SEQ ID NO: 626 | and T4 Fibritin Foldon domain (27 amino acids; SEQ ID NO: 45) is |
| | added to the 847B mutant ectodomain via an SAIG linker to form a 574 |
| | aa mutant polypeptide |

TABLE 27
RSV F modRNA constructs

| | | | | | RSV F |
|---|---|---|---|---|---|
| | 5′-UTR* | RSV [ORF] | 3′-UTR | Poly-A tail** | SEQ ID |
| RNA | SEQ ID NO | SEQ ID NO | SEQ ID NO | SEQ ID NO | NO |
| Construct | [DNA/RNA] | [DNA/RNA] | [DNA/RNA] | [DNA/RNA] | [Protein] |
| RSV F | 87/373 | 8/15 | 281/567 | 21/22 | 4 |
| 847A_5UTR_563/3UTR_2 | | | | | |
| 80A modRNA | | | | | |
| RSV F | 87/373 | 10/16 | 281/567 | 21/22 | 6 |
| 847B_5UTR_563/3UTR_2 | | | | | |
| 80A modRNA | | | | | |
| RSV F | 94/380 | 8/15 | 281/567 | 21/22 | 4 |
| 847A_5UTR_582/3UTR_2 | | | | | |
| 80A modRNA | | | | | |
| RSV F 847B | 94/380 | 10/16 | 281/567 | 21/22 | 6 |
| 5UTR_582/3UTR_2 | | | | | |
| 80A modRNA | | | | | |
| RSV F | 99/385 | 8/15 | 281/567 | 21/22 | 4 |
| 847A_5UTR_599/3UTR_2 | | | | | |
| 80A modRNA | | | | | |
| RSV F | 99/385 | 10/16 | 281/567 | 21/22 | 6 |
| 847B_5UTR_599/3UTR_2 | | | | | |
| 80A modRNA | | | | | |
| RSV F | 87/373 | 627/633 | 281/567 | 21/22 | 621 |
| 851A_5UTR_563/3UTR_2 | | | | | |
| 80A modRNA | | | | | |
| RSV F | 87/373 | 628/634 | 281/567 | 21/22 | 622 |
| 851B_5UTR_563/3UTR_2 | | | | | |
| 80A modRNA | | | | | |
| RSV F | 94/380 | 627/633 | 281/567 | 21/22 | 621 |
| 851A_5UTR_582/3UTR_2 | | | | | |
| 80A modRNA | | | | | |
| RSV F 851B | 94/380 | 628/634 | 281/567 | 21/22 | 622 |
| 5UTR_582/3UTR_2 | | | | | |
| 80A modRNA | | | | | |
| RSV F | 99/385 | 627/633 | 281/567 | 21/22 | 621 |
| 851A_5UTR_599/3UTR_2 | | | | | |
| 80A modRNA | | | | | |
| RSV F | 99/385 | 628/634 | 281/567 | 21/22 | 622 |
| 851B_5UTR_599/3UTR_2 | | | | | |
| 80A modRNA | | | | | |
| RSV F | 87/373 | 629/635 | 281/567 | 21/22 | 623 |
| 852A_5UTR_563/3UTR_2 | | | | | |
| 80A modRNA | | | | | |
| RSV F | 87/373 | 630/636 | 281/567 | 21/22 | 624 |
| 852B_5UTR_563/3UTR_2 | | | | | |
| 80A modRNA | | | | | |
| RSV F | 94/380 | 629/635 | 281/567 | 21/22 | 623 |
| 852A_5UTR_582/3UTR_2 | | | | | |
| 80A modRNA | | | | | |
| RSV F 852B | 94/380 | 630/636 | 281/567 | 21/22 | 624 |
| 5UTR_582/3UTR_2 | | | | | |
| 80A modRNA | | | | | |
| RSV F | 99/385 | 629/635 | 281/567 | 21/22 | 623 |
| 852A_5UTR_599/3UTR_2 | | | | | |
| 80A modRNA | | | | | |
| RSV F | 99/385 | 630/636 | 281/567 | 21/22 | 624 |
| 852B_5UTR_599/3UTR_2 | | | | | |
| 80A modRNA | | | | | |
| RSV F 847A Ecto- | 87/373 | 631/637 | 281/567 | 21/22 | 625 |
| Foldon_5UTR_563/3UTR_2 | | | | | |
| 80A modRNA | | | | | |
| RSV F 847B Ecto- | 87/373 | 632/638 | 281/567 | 21/22 | 626 |
| Foldon_5UTR_563/3UTR_2 | | | | | |
| 80A modRNA | | | | | |
| RSV F 847A Ecto- | 94/380 | 631/637 | 281/567 | 21/22 | 625 |
| Foldon_5UTR_582/3UTR_2 | | | | | |
| 80A modRNA | | | | | |
| RSV F 847B Ecto- | 94/380 | 632/638 | 281/567 | 21/22 | 626 |
| Foldon | | | | | |
| 5UTR_582/3UTR_280A | | | | | |
| modRNA | | | | | |
| RSV F 847A Ecto- | 99/385 | 631/637 | 281/567 | 21/22 | 625 |
| Foldon_5UTR_599/3UTR_2 | | | | | |
| 80A modRNA | | | | | |
| RSV F 847B Ecto- | 99/385 | 632/638 | 281/567 | 21/22 | 626 |
| Foldon_5UTR_599/3UTR_2 | | | | | |
| 80A modRNA | | | | | |

*5′ UTR sequence includes 5′ cap sequence
**Poly-A tail length may contain +1/−1 A

2. hMPV preF modRNA Constructs

RNA constructs generated herein (Table 29) encode hMPV F protein wild-type (WT) and hMPV F protein variants/mutants (e.g. hMPV pre-fusion F protein). Table 28 describes WT F (WT 5 F) and variant hMPV preF mutants.

TABLE 28
hMPV F proteins and description

| hMPV F Protein | |
|---|---|
| Sequence | hMPV F Protein Description |
| hMPV A WT F | Full Length F0 of Native hMPV A2b (GenBank GI: ACJ53569.1) (539 |
| SEQ ID NO: 639 | aa) |
| hMPV B WT F | Full Length F0 of Native hMPV B2 (GenBank GI: QDA18370.1) (539 |
| SEQ ID NO: 640 | aa) |
| hMPV F hMPV021 A | Full length hMPV subtype A hMPV021 mutant F protein with |
| SEQ ID NO: 641 | substitutions Q100R, S101R, and D185P (539 aa) |
| hMPV F hMPV029 A | Full length hMPV subtype A hMPV029 mutant F protein with |
| SEQ ID NO: 642 | substitutions Q100R, S101R, L110C, T127C, A140C, A147C, N153C, |
| | D185P, L219K, V231I, N322C, T365C, E453Q, and V463C (539 aa) |
| hMPV F hMPV198 A | Full length hMPV subtype A hMPV198 mutant F protein with |
| SEQ ID NO: 643 | substitutions V84C, deletion of residues at positions 89-112 replaced |
| | with a GSGGSG linker, A140C, A147C, D185P, A249C, D454C, and |
| | V458C (521 aa) |
| hMPV F hMPV189 B | Full length hMPV subtype B hMPV189 mutant F protein with |
| SEQ ID NO: 644 | substitutions Q100R, S101R, L110C, T127C, A140C, A147C, N153C, |
| | A185P, L219K, V231I, N322C, T365C, E453Q, and V463C (539 aa) |
| hMPV F hMPV194 B | Full length hMPV subtype B hMPV194 mutant F protein with |
| SEQ ID NO: 645 | substitutions V84C, deletion of residues at positions 89-112 replaced |
| | with a GSGGSG linker, A140C, A147C, A185P, A249C, D454C, and |
| | V458C (521 aa) |
| hMPV F hMPV029 A | hMPV subtype A hMPV029 mutant F protein with substitutions |
| Ecto-foldon | Q100R, S101R, L110C, T127C, A140C, A147C, N153C, D185P, |
| SEQ ID NO: 646 | L219K, V231I, N322C, T365C, E453Q, and V463C; truncated after |
| | position 489 (TM and CT removed) and T4 Fibritin Foldon domain (27 |
| | amino acids; SEQ ID NO: 45) is added to the hMPV029 mutant |
| | ectodomain via an SAIG linker to form a 520 aa mutant polypeptide |
| hMPV F hMPV189 B | hMPV subtype B hMPV189 mutant F protein with substitutions |
| Ecto-foldon | Q100R, S101R, L110C, T127C, A140C, A147C, N153C, A185P, |
| SEQ ID NO: 647 | L219K, V231I, N322C, T365C, E453Q, and V463C; truncated after |
| | position 489 (TM and CT removed) and T4 Fibritin Foldon domain (27 |
| | amino acids; SEQ ID NO: 45) is added to the hMPV189 mutant |
| | ectodomain via an SAIG linker to form a 520 aa mutant polypeptide |
| hMPV F hMPV198 A | hMPV subtype A hMPV198 mutant F protein with substitutions V84C, |
| Ecto-foldon | d89-112 replaced with GSGGSG Linker, A140C, A147C, D185P, |
| SEQ ID NO: 715 | A249C, D454C, and V458C; truncated after position 489 (TM and CT |
| | removed) and T4 Fibritin Foldon domain (27 amino acids; SEQ ID NO: |
| | 45) is added to the hMPV198 mutant ectodomain via an SAIG linker |
| | to form a 502 aa mutant polypeptide |
| hMPV F hMPV194 B | hMPV subtype B hMPV194 mutant F protein with V84C, d89-112 |
| Ecto-foldon | replaced with GSGGSG Linker, A140C, A147C, A185P, A249C, |
| SEQ ID NO: 716 | D454C, and V458C; truncated after position 489 (TM and CT |
| | removed) and T4 Fibritin Foldon domain (27 amino acids; SEQ ID NO: |
| | 45) is added to the hMPV194 mutant ectodomain via an SAIG linker |
| | to form a 502 aa mutant polypeptide |
| hMPV F hMPV164 B | Full length hMPV subtype B hMPV164 mutant F protein with |
| SEQ ID NO: 721 | substitutions Q100R, S101R, and A185P (539 aa) |

TABLE 29
hMPV F modRNA constructs

| | | | | | hMPV F |
|---|---|---|---|---|---|
| | 5′-UTR* | hMPV [ORF] | 3′-UTR | Poly-A tail** | SEQ ID |
| RNA | SEQ ID NO | SEQ ID NO | SEQ ID NO | SEQ ID NO | NO |
| Construct | [DNA/RNA] | [DNA/RNA] | [DNA/RNA] | [DNA/RNA] | [Protein] |
| hMPV029_5UTR_582/ | 94/380 | 650/662 | 281/567 | 21/22 | 642 |
| 3UTR_2 80A | | | | | |
| modRNA | | | | | |
| hMPV198_5UTR_582/ | 94/380 | 651/663 | 281/567 | 21/22 | 643 |
| 3UTR_2 80A | | | | | |
| modRNA | | | | | |
| hMPV189_5UTR_582/ | 94/380 | 653/664 | 281/567 | 21/22 | 644 |
| 3UTR_2 80A | | | | | |
| modRNA | | | | | |
| hMPV194_5UTR_582/ | 94/380 | 654/665 | 281/567 | 21/22 | 645 |
| 3UTR_2 80A | | | | | |
| modRNA | | | | | |
| hMPV021_5UTR_562/ | 86/372 | 649/661 | 281/567 | 21/22 | 641 |
| 3UTR_2 80A | | | | | |
| modRNA | | | | | |
| hMPV021_5UTR_563/ | 87/373 | 649/661 | 281/567 | 21/22 | 641 |
| 3UTR_2 80A | | | | | |
| modRNA | | | | | |
| hMPV021_5UTR_576/ | 88/374 | 649/661 | 281/567 | 21/22 | 641 |
| 3UTR_2 80A | | | | | |
| modRNA | | | | | |
| hMPV021_5UTR_579/ | 71/357 | 649/661 | 281/567 | 21/22 | 641 |
| 3UTR_2 80A | | | | | |
| modRNA | | | | | |
| hMPV021_5UTR_581/ | 92/378 | 649/661 | 281/567 | 21/22 | 641 |
| 3UTR_2 80A | | | | | |
| modRNA | | | | | |
| hMPV021_5UTR_582/ | 94/380 | 649/661 | 281/567 | 21/22 | 641 |
| 3UTR_2 80A | | | | | |
| modRNA | | | | | |
| hMPV021_5UTR_583/ | 98/384 | 649/661 | 281/567 | 21/22 | 641 |
| 3UTR_2 80A | | | | | |
| modRNA | | | | | |

*5′ UTR sequence includes 5′ cap sequence
**Poly-A tail length may contain +1/−1 A

3. PIV1 preF modRNA Constructs

RNA constructs generated herein (Table 31) encode PIV1 protein wild-type (WT) and PIV1 F protein variants/mutants (e.g. PIV1 pre-fusion F protein). Table 30 describes WT F (WT F) and variant PIV1 preF mutants.

TABLE 30
PIV1 F proteins and description

| PIV1 F Protein | |
|---|---|
| Sequence | PIV1 F Protein Description |
| PIV1 WT F | Full Length F0 of Native PIV1 (GenBank GI: AFP49460.1) (555 aa) |
| SEQ ID NO: 672 | |
| PIV1 F PIV1047 | Full length PIV1 PIV1047 mutant F protein with substitutions Q92C, |
| SEQ ID NO: 673 | F113G, F114S, G134C, A466L, S473L, and A480L (555 aa) |
| PIV1 F PIV1069 | Full length PIV1 PIV1069 mutant F protein with substitutions F113G, |
| SEQ ID NO: 674 | F114S, G134A, A466L, and S473L (555 aa) |
| PIV1 F PIV1047 | PIV1 PIV1047 mutant F protein with substitutions Q92C, F113G, |
| Ecto-Foldon | F114S, G134C, A466L, S473L, and A480L; is truncated after position |
| SEQ ID NO: 675 | 480 (TM and CT removed) and T4 Fibritin Foldon domain (27 amino |
| | acids; SEQ ID NO: 45) is added to the PIV1047 mutant ectodomain via |
| | an SAIG linker to form a 511 aa mutant polypeptide |
| PIV1 F PIV1069 | PIV1 PIV1069 mutant F protein with substitutions F113G, F114S, |
| Ecto-Foldon | G134A, A466L, and S473L; is truncated after position 477 (TM and CT |
| SEQ ID NO: 676 | removed) and T4 Fibritin Foldon domain (27 amino acids; SEQ ID NO: |
| | 45) is added to the PIV1069 mutant ectodomain via an SAIG linker to |
| | form a 508 aa mutant polypeptide |

TABLE 31
PIV1 F modRNA constructs

| | | PIV1 F | | | PIV1 F |
|---|---|---|---|---|---|
| | 5′-UTR* | [ORF] | 3′-UTR | Poly-A tail** | SEQ ID |
| RNA | SEQ ID NO | SEQ ID NO | SEQ ID NO | SEQ ID NO | NO |
| Construct | [DNA/RNA] | [DNA/RNA] | [DNA/RNA] | [DNA/RNA] | [Protein] |
| PIV1047_5UTR_576/ | 88/374 | 678/684 | 281/567 | 21/22 | 673 |
| 3UTR_2 80A | | | | | |
| modRNA | | | | | |
| PIV1069_5UTR_576/ | 88/374 | 679/685 | 281/567 | 21/22 | 674 |
| 3UTR_2 80A | | | | | |
| modRNA | | | | | |
| PIV1047_5UTR_562/ | 86/372 | 678/684 | 281/567 | 21/22 | 673 |
| 3UTR_2 80A | | | | | |
| modRNA | | | | | |
| PIV1047_5UTR_579/ | 71/357 | 678/684 | 281/567 | 21/22 | 673 |
| 3UTR_2 80A | | | | | |
| modRNA | | | | | |
| PIV1047_5UTR_582/ | 94/380 | 678/684 | 281/567 | 21/22 | 673 |
| 3UTR_2 80A | | | | | |
| modRNA | | | | | |
| PIV1047_5UTR_583/ | 98/384 | 678/684 | 281/567 | 21/22 | 673 |
| 3UTR_2 80A | | | | | |
| modRNA | | | | | |

*5′ UTR sequence includes 5′ cap sequence
**Poly-A tail length may contain +1/−1 A

4. PIV1 HN modRNA Constructs

RNA constructs are set forth in Table 33 which encode PIV1 HN protein wild-type (WT) and PIV1 HN protein variants/mutants. Table 32 describes WT PIV1 HN (WT HN) and variant PIV1 HN mutants.

TABLE 32
PIV1 HN proteins and description

| PIV1 HN Protein | |
|---|---|
| Sequence | PIV1 HN Protein Description |
| PIV1 WT HN | Full length of WT PIV1 HN protein (GenBank GI: ATI99865.1) (575 |
| PIV1083 | aa) |
| SEQ ID NO: 750 | |
| PIV1 HN PIV1084 | Full length PIV1 PIV1084 mutant HN protein with deletion of residues |
| SEQ ID NO: 756 | at positions 57-84 of SEQ ID NO: 750 (547 aa) |
| PIV1 HN PIV1085 | Full length PIV1 PIV1085 mutant HN protein with deletion of residues |
| SEQ ID NO: 757 | at positions 57-129 at SEQ ID NO: 750 (502 aa) |

TABLE 33
PIV1 HN modRNA constructs

| | | PIV1 HN | | | PIV1 HN |
|---|---|---|---|---|---|
| | 5′-UTR* | [ORF] | 3′-UTR | Poly-A tail** | SEQ ID |
| RNA | SEQ ID NO | SEQ ID NO | SEQ ID NO | SEQ ID NO | NO |
| Construct | [DNA/RNA] | [DNA/RNA] | [DNA/RNA] | [DNA/RNA] | [Protein] |
| PIV1083_5UTR_579/ | 71/357 | 758/761 | 281/567 | 21/22 | 750 |
| 3UTR_2 80A modRNA | | | | | |
| PIV1084_5UTR_579/ | 71/357 | 759/762 | 281/567 | 21/22 | 756 |
| 3UTR_2 80A | | | | | |
| PIV1085_5UTR_579/ | 71/357 | 760/763 | 281/567 | 21/22 | 757 |
| 3UTR_2 80A modRNA | | | | | |
| PIV1083_5UTR_582/ | 94/380 | 758/761 | 281/567 | 21/22 | 750 |
| 3UTR_2 80A modRNA | | | | | |
| PIV1084_5UTR_582/ | 94/380 | 759/762 | 281/567 | 21/22 | 756 |
| 3UTR_2 80A modRNA | | | | | |
| PIV1085_5UTR_582/ | 94/380 | 760/763 | 281/567 | 21/22 | 757 |
| 3UTR_2 80A modRNA | | | | | |

*5′ UTR sequence includes 5′ cap sequence
**Poly-A tail length may contain +1/−1 A

5. PIV3 preF modRNA Constructs

RNA constructs generated herein (Table 35) encode PIV3 protein wild-type (WT) and PIV3 F protein variants/mutants (e.g. PIV3 pre-fusion F protein). Table 34 describes WT F (WT F) and variant PIV3 preF mutants.

TABLE 34
PIV3 F proteins and description

| PIV3 F Protein | |
|---|---|
| Sequence | PIV3 F Protein Description |
| PIV3 WT F | Full length F0 of Native PIV3, F protein (GenBank GI: AGT75285.1) |
| SEQ ID NO: 690 | (539 aa) |
| PIV3 F PIV3135 | Full length PIV3 PIV3135 mutant F protein with substitutions E209C |
| SEQ ID NO: 691 | and L234C (539 aa) |
| PIV3 F PIV3140 | Full length PIV3 PIV3140 mutant F protein with substitutions S160C, |
| SEQ ID NO: 692 | V170C, E209C, L234C, A463L, and S470L (539 aa) |
| PIV3 F PIV3135 | PIV3 PIV3135 mutant F protein with substitutions E209C and L234C; |
| Ecto-Foldon | is truncated after position 481 (TM and CT removed) and T4 Fibritin |
| SEQ ID NO: 693 | Foldon domain (27 amino acids; SEQ ID NO: 45) is added to the |
| | PIV3135 mutant ectodomain via an SAIG linker to form a 512 aa mutant |
| | polypeptide |
| PIV3 F PIV3008 | Full length PIV3 PIV3008 mutant F protein with substitutions Q162C, |
| SEQ ID NO: 712 | L168C, I213C, G230C, A463V, and I474Y (539 aa) |
| PIV3 F PIV3140 | PIV3 PIV3140 mutant F protein with substitutions S160C, V170C, |
| Ecto-Foldon | E209C, L234C, A463L, and S470L; is truncated after position 481 (TM |
| SEQ ID NO: 694 | and CT removed) and T4 Fibritin Foldon domain (27 amino acids; SEQ |
| | ID NO: 45) is added to the PIV3140 mutant ectodomain via an SAIG |
| | linker to form a 512 aa mutant polypeptide |

TABLE 35
PIV3 F modRNA constructs

| | | | | | PIV3 F |
|---|---|---|---|---|---|
| | 5′-UTR* | PIV3 F [ORF] | 3′-UTR | Poly-A tail** | SEQ ID |
| RNA | SEQ ID NO | SEQ ID NO | SEQ ID NO | SEQ ID NO | NO |
| Construct | [DNA/RNA] | [DNA/RNA] | [DNA/RNA] | [DNA/RNA] | [Protein] |
| PIV3135_5UTR_579/ | 71/357 | 696/704 | 281/567 | 21/22 | 691 |
| 3UTR_2 80A | | | | | |
| modRNA | | | | | |
| PIV3140_5UTR_579/ | 71/357 | 697/705 | 281/567 | 21/22 | 692 |
| 3UTR_2 80A | | | | | |
| modRNA | | | | | |
| PIV3135_5UTR_582/ | 94/380 | 696/704 | 281/567 | 21/22 | 691 |
| 3UTR_2 80A | | | | | |
| modRNA | | | | | |
| PIV3140_5UTR_582/ | 94/380 | 697/705 | 281/567 | 21/22 | 692 |
| 3UTR_2 80A | | | | | |
| modRNA | | | | | |
| PIV3008_5UTR_562/ | 86/372 | 713/714 | 281/567 | 21/22 | 712 |
| 3UTR_2 80A | | | | | |
| modRNA | | | | | |
| PIV3008_5UTR_563/ | 87/373 | 713/714 | 281/567 | 21/22 | 712 |
| 3UTR_2 80A | | | | | |
| modRNA | | | | | |
| PIV3008_5UTR_576/ | 88/374 | 713/714 | 281/567 | 21/22 | 712 |
| 3UTR_2 80A | | | | | |
| modRNA | | | | | |
| PIV3008_5UTR_579/ | 71/357 | 713/714 | 281/567 | 21/22 | 712 |
| 3UTR_2 80A | | | | | |
| modRNA | | | | | |
| PIV3008_5UTR_582/ | 94/380 | 713/714 | 281/567 | 21/22 | 712 |
| 3UTR_2 80A | | | | | |
| modRNA | | | | | |
| PIV3008_5UTR_583/ | 98/384 | 713/714 | 281/567 | 21/22 | 712 |
| 3UTR_2 80A | | | | | |
| modRNA | | | | | |

*5′ UTR sequence includes 5′ cap sequence
**Poly-A tail length may contain +1/−1 A

6. PIV3 HN modRNA Constructs

RNA constructs generated herein (Table 37) encode PIV3 HN protein wild-type (WT) and PIV3 HN protein variants/mutants. Table 36 describes WT HN (WT HN) and variant PIV3 HN mutants.

TABLE 36
PIV3 HN proteins and description

| PIV3 HN Protein | |
|---|---|
| Sequence | PIV3 HN Protein Description |
| PIV3 WT HN | Full length of WT PIV3 HN protein (GenBank GI: AGT75286.1) (572 aa) |
| PIV3223 | |
| SEQ ID NO: 724 | |
| PIV3 HN | Full length PIV3 PIV3224 mutant HN protein with deletion of residues at |
| PIV3224 | positions 59-88 of SEQ ID NO: 724 (542 aa) |
| SEQ ID NO: 725 | |
| PIV3 HN | Full length PIV3 PIV3225 mutant HN protein with deletion of residues at |
| PIV3225 | positions 59-130 of SEQ ID NO: 724 (500 aa) |
| SEQ ID NO: 726 | |

TABLE 37
PIV3 HN modRNA constructs

| | | PIV3 HN | | | PIV3 HN |
|---|---|---|---|---|---|
| | 5′-UTR* | [ORF] | 3′-UTR | Poly-A tail** | SEQ ID |
| RNA | SEQ ID NO | SEQ ID NO | SEQ ID NO | SEQ ID NO | NO |
| Construct | [DNA/RNA] | [DNA/RNA] | [DNA/RNA] | [DNA/RNA] | [Protein] |
| PIV3223_5UTR_579/ | 71/357 | 727/736 | 281/567 | 21/22 | 724 |
| 3UTR_2 80A modRNA | | | | | |
| PIV3224_5UTR_579/ | 71/357 | 728/737 | 281/567 | 21/22 | 725 |
| 3UTR_2 80A | | | | | |
| PIV3225_5UTR_579/ | 71/357 | 729/738 | 281/567 | 21/22 | 726 |
| 3UTR_2 80A modRNA | | | | | |
| PIV3223_5UTR_582/ | 94/380 | 727/736 | 281/567 | 21/22 | 724 |
| 3UTR_2 80A modRNA | | | | | |
| PIV3224_5UTR_582/ | 94/380 | 728/737 | 281/567 | 21/22 | 725 |
| 3UTR_2 80A modRNA | | | | | |
| PIV3225_5UTR_582/ | 94/380 | 729/738 | 281/567 | 21/22 | 726 |
| 3UTR_2 80A modRNA | | | | | |

*5′ UTR sequence includes 5′ cap sequence
**Poly-A tail length may contain +1/−1 A

### Example 2: modRNA Formulation into mRNA-Lipid-Nanoparticle (modRNA-LNP) for LNP1

The LNP1 formulation contains 2 functional lipids, ALC-0315 (ionizable cationic lipid) and ALC-0159 (PEG-lipid), and 2 structural lipids DSPC (1,2distearoyl-sn-glycero-3-phosphocholine) and cholesterol. The physicochemical properties and the structures of the 4 lipids are shown in Table 38 below.

Lipid nanoparticles were prepared and tested according to the general procedures described in U.S. Pat. No. 9,737,619 (PCT Pub. No. WO2015/199952) and U.S. Pat. No. 10,166,298 (WO 2017/075531) and WO2020/146805, each of which is hereby incorporated by reference in its entirety.

Briefly, modRNA-LNPs were formulated by combining a modRNA-containing aqueous phase and a lipid-containing organic phase using a T-mixer. The organic phase was prepared by solubilizing a mixture of ionizable lipid, phospholipid, polyethylene glyco-lipid, and cholesterol at a pre-determined ratio in ethanol (e.g., at a molar ratio of about 47.5:10:40.7:1.8). The organic phase and aqueous phase were mixed by syringe pumps. The resultant solution was dialyzed against 10 mM Tris buffer (pH 7.4). Post-dialysis solution was concentrated and spiked with cryoprotectant to form a final modRNA-LNP solution.

TABLE 38
Lipids in the LNP1 Formulation

| | Molecular | Molecular Formula | |
|---|---|---|---|
| Lipid (CAS | Weight | Physical state and | |
| number) | [Da] | Storage condition | Chemical name and structure |
| Cationic Lipid ALC-0315 (Not applicable) | 766 | C₄₈H₉₅NO₅ Liquid (oil) −20° C. | (4-hydroxybutyl)azanediyl)bis(hexane-6,1- diyl)bis(2-hexyldecanoate 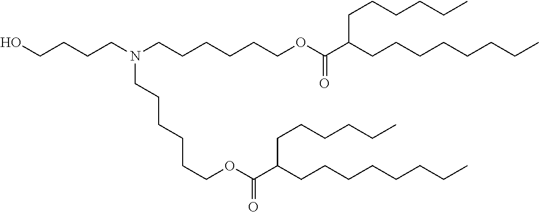 [SMILES: CCCCCCCCC(CCCCCC)C(=O)OCCCCCCN(CCCCO)CCCCCCOC(=O)C(CCCCCC)CCCCCCCC] |
| PEG-Lipid ALC-0159 (1849616- 42-7) | ~2400- 2600 | C₃₀H₆₀NO(C₂H₄O)NOCH₃ n = 45-50 Solid −20° C. | 2-[(polyethylene gylcol)-2000]-N,N- ditetradecyclacetamide 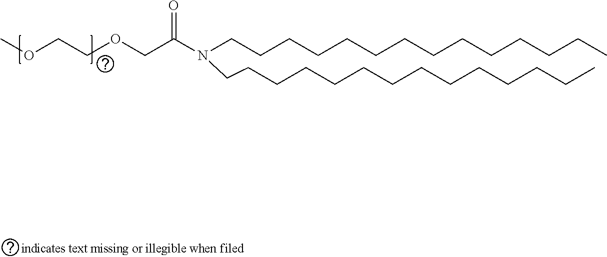 |
| DSPC (816-94-4) | 790 | C₃₀H₈₈NO₈P Solid −20° C. | 1,2-Distearoyl-sn-glycero-3-phosphocholine 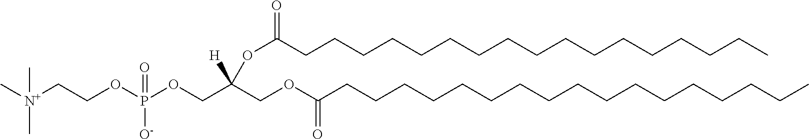 [SMILES: CCCCCCCCCCCCCCCCCC(=O)OC[C@H](COP(=O)([O-])OCC[N+](C)(C)C)OC(=O)CCCCCCCCCCCCCCCCC] |
| Cholesterol (57-88-5) | 387 | C₂₇H₄₆O Solid −20° C. | 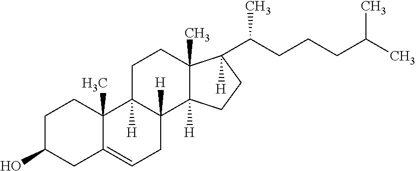 |

CAS = Chemical Abstract Service; DSPC = 1,2-disteroyl-sn-glycero-3-phosphocholine

### Example 3. Evaluation of Different UTR Designs in modRNA Encoding Full-Length RSV F Protein in Mice

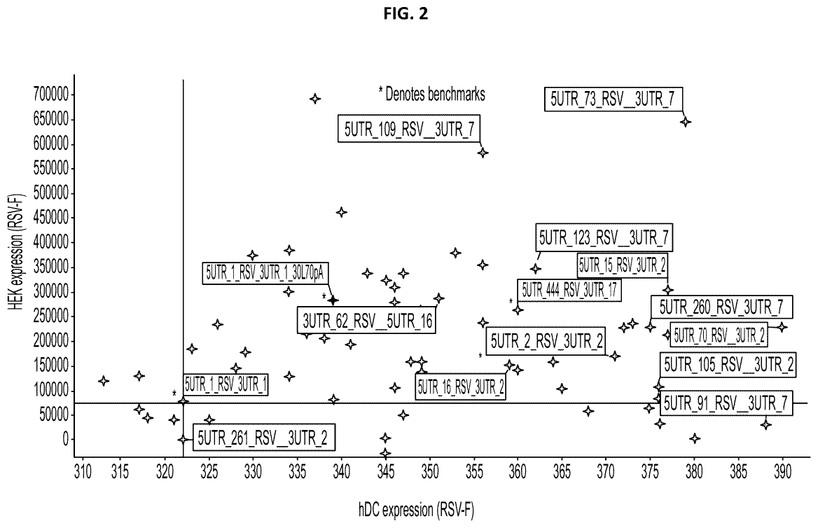

135 unique UTR constructs were tested for RSV-F expression in both HEK (human embryonic kidney) cells and hDCs (human dendritic cells) in multiple subsets. A representative dataset of 64 constructs were tested for RSV-F expression (FIG. 2) in both HEK and hDCs. A majority of the UTR constructs yielded higher expression compared to the WHO UTRs (5UTR_1_RSV_3UTR_1) (5′UTR sequence SEQ ID NO: 111 and 3′UTR sequence SEQ ID NO: 280) and the human hemoglobin (5UTR_2_RSV_3UTR_2) (5′UTR sequence SEQ ID NO: 73 and 3′UTR sequence SEQ ID NO: 281) benchmarks.

Based on expression in HEK and hDC cells, 11 constructs were chosen for testing by assessing immunogenicity (neutralizing antibody response) of RSV-F encoding modRNA-LNPs in multiple rounds of screening. Not all sequences from the in silico design methods worked equally well. A low-performing UTR, 5UTR_261_RSV_3UTR_2 (5′ UTR sequence SEQ ID NO: 59), was included as a low-expression control, and to evaluate translation of results from in vitro protein expression to in vivo immunogenicity (FIG. 2, labeled constructs). Drug substance and subsequently drug product was generated for a total of 15 constructs which included the four benchmarks: WHO (5UTR_1/3UTR_1, or SEQ ID NO: 111 and SEQ ID NO:280) with both the contiguous (80A) and the interrupted (30L⁷⁰, consisting of 30A, then the linker of SEQ ID NO: 23, then an additional 70A) polyA, another marketed modRNA UTRs as benchmarks (5UTR_444, SEQ ID NO:90 and 3UTR_17, SEQ ID NO:287), and human hemoglobin UTRs (5UTR_2 and 3UTR_2, or SEQ ID NO:73 and SEQ ID NO:281) (Table 39).

TABLE 39
Quality attributes of RSV modRNA LNPs generated for in vivo study

| | | | | LNP | | | |
|---|---|---|---|---|---|---|---|
| Drug substance | | % | % | size | | RNA conc | Encapsulation |
| construct | UTR construct | Integrity | Capped | (nm) | PDI | (ng/ul) | % |
| UTR-OPT-1027 | WHO/WHO-80A | 91.20 | 86.70 | 80.9 | 0.24 | 10.134 | 93 |
| UTR-OPT-1028 | hHBB/hHBB-80A | 93.20 | 96.40 | 67.4 | 0.13 | 0.156 | 97 |
| UTR-OPT-1030 | 5UTR_261/hHBB-80A | 84.10 | 94.70 | 60.7 | 0.08 | 0.174 | 98 |
| UTR-OPT-1032 | 5UTR_260/hHBB-80A | 86.70 | 94.50 | 68.1 | 0.17 | 0.146 | 97 |
| UTR-OPT-1036 | 5UTR_15/hHBB-80A | 83.70 | 94.60 | 61.2 | 0.09 | 0.149 | 99 |
| UTR-OPT-1037 | 5UTR_16/hHBB-80A | 90.90 | 92.50 | 64.2 | 0.12 | 0.151 | 98 |
| mRNA-RSV-0012 | 5UTR_70/hHBB-80A | 92.70 | 93.80 | 63.2 | 0.13 | 0.154 | 99 |
| mRNA-RSV-0019 | 5UTR_105/hHBB-80A | 91.10 | 94.80 | 56.2 | 0.06 | 0.148 | 99 |
| mRNA-RSV-0037 | 5UTR_73/3UTR_7-80A | 92.80 | 93.80 | 71.2 | 0.14 | 0.142 | 91 |
| mRNA-RSV-0039 | 5UTR_91/3UTR_7 -80A | 92.60 | 96.20 | 64.2 | 0.19 | 0.157 | 98 |
| mRNA-RSV-0041 | 5UTR_109/3UTR_7 -80A | 92.40 | 94.50 | 60.6 | 0.09 | 0.15 | 99 |
| mRNA-RSV-0043 | 5UTR_123/3UTR_7-80A | 91.90 | 97.20 | 60.4 | 0.07 | 0.155 | 98 |
| mRNA-RSV-0050 | 5UTR_16/hHBB_3AES-80A | 90.90 | 94.80 | 71.8 | 0.17 | 0.13 | 90 |
| mRNA-RSV-0051 | WHO/WHO-30L70A | 83.20 | 82.80 | 65.7 | 0.13 | 0.132 | 98 |
| UTR-OPT-1094 | 5UTR_444/3UTR_17-80A | 90.1 | 92.80 | 58.7 | 0.1 | 0.19 | 97 |

All modRNA LNPs were of high quality with >80% full length capped RNAs, with high encapsulation efficiency within lipid nanoparticles of low polydispersity index (Table 39). Furthermore, in vitro expression of these modRNA LNPs was tested in HEK293T cells and hDCs (Table 40).

TABLE 40
In vitro expression profile of UTR candidates for RSV-F mouse study

| | | HEK293T | |
|---|---|---|---|
| | | Relative | hDC |
| | | Expression | Relative |
| Name | UTR construct | (run2) | Expression |
| UTR-OPT-1027 | WHO_WHO_80pA | 100 | 100 |
| UTR-OPT-1028 | hHBB_hHBB | 70.86 | 107.58 |
| UTR-OPT-1030 | 5UTR_261_RSV_3UTR_hHBB | 2.01 | 10.93 |
| UTR-OPT-1032 | 5UTR_260_RSV_3UTR_7 | 80.08 | 181.1 |
| UTR-OPT-1036 | 5UTR_15_RSV_3UTR_hHBB | 84.91 | 160.38 |
| UTR-OPT-1037 | 5UTR_16_RSV_3UTR_hHBB | 76.65 | 152.1 |
| mRNA-RSV-0012 | 5UTR_70_RSV_3UTR_hHBB | 86.03 | 175.53 |
| mRNA-RSV-0037 | 5UTR_73_RSV_3UTR_7 | 110.06 | 298.7 |
| mRNA-RSV-0039 | 5UTR_91_RSV_3UTR_7 | 59.88 | 129.68 |
| mRNA-RSV-0043 | 5UTR_123_RSV_3UTR_7 | 62.74 | 183.08 |
| mRNA-RSV-0019 | 5UTR_105_RSV_3UTR_hHBB | 68.88 | 170.78 |
| mRNA-RSV-0041 | 5UTR_109_RSV_3UTR_7 | 68.17 | 255.49 |
| mRNA-RSV-0050 | 5UTR_16_RSV_3hHBB_3AES | 44.87 | 263.3 |
| mRNA-RSV-0051 | WHO_WHO_30L70 | 61.76 | 151.63 |
| UTR-OPT-1094 | 5UTR_444_RSV_3UTR_17 | 45.6 | 55.32 |

There was a reasonable agreement in performance of the 5′ UTRs in HEK cells in both of the experiments (FIG. 2 and Table 40), e.g., the highest expression in both experiments was observed for mRNA-RSV-0037 (5UTR_73/3UTR_7). Additionally, the low expressing control UTR-OPT-1030 (5UTR_261/3UTR_2) also performed similarly in both experiments. In human dendritic cells, the novel UTR constructs performed better than the WHO benchmark with 80 polyA, except for the low expressing control, which was only about 10% as potent.

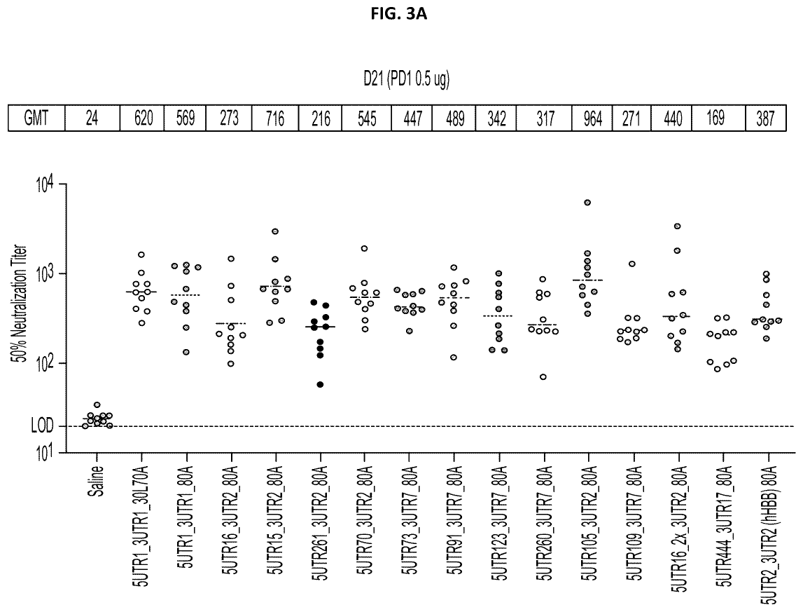

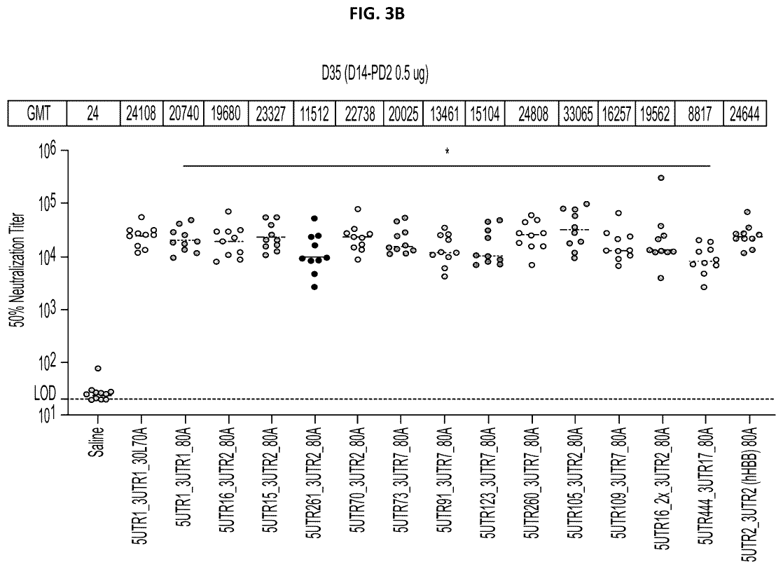

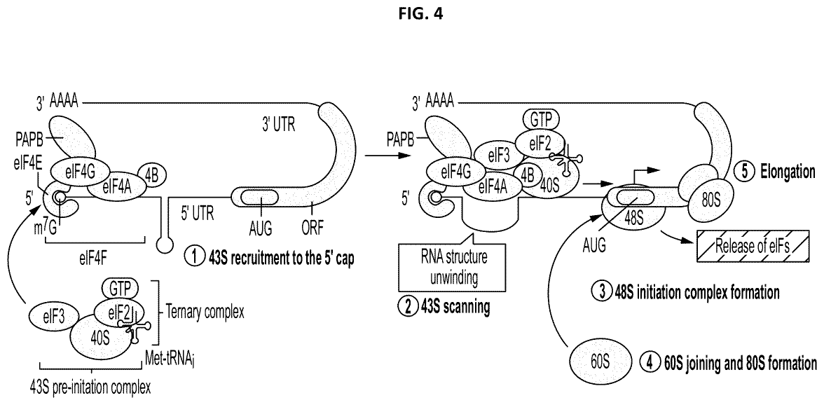

Drug products were then transferred for immunogenicity testing in mice. Groups of female BALB/C mice received RSV-F modRNA-LNPs representing different UTR candidates intramuscularly on day 0 and day 21. Sera collected on day 21 (post dose 1, PD1) and day 35 (14 days PD2) were assessed for RSV neutralizing response. After the first dose, it was found that several top performing UTRs from the in vitro screening induced higher or similar levels of 50% neutralizing titers compared to several benchmark UTRs including the human hemoglobin (hHBB 5UTR_2/3UTR_2), WHO (5UTR_1/3UTR_1), and marketed Covid-19 vaccine modRNA benchmark UTRs with both contiguous and interrupted polyA (FIG. 3A). Particularly, 5UTR_105 (SEQ ID NO:55) and 5UTR_15 (SEQ ID NO:50) had higher neutralizing titer levels than at least one of the WHO benchmarks, albeit not statistically significant. Consistent with the in vitro expression results, the low expressing control 5UTR_261 (SEQ ID NO:59)/hHBB (SEQ ID NO: 281) showed lower levels of neutralizing antibody titers. The UTR benchmark (5UTR_444/3UTR_17) based on a marketed vaccine modRNA also showed poor response in mice which was even lower than the 5UTR_261 low-performing control.

Interestingly, after the second dose (day 35), other novel UTRs induced a neutralizing response comparable to top-performing UTRs in post-dose 1 (FIG. 3B). For example, the 5UTR_260 (SEQ ID NO:58) and the hHBB benchmark (5UTR_2, SEQ ID NO:73) showed similar neutralizing antibody responses as novel-designed UTRs such as 5UTR_15 (SEQ ID NO:50) and 5UTR_105 (SEQ ID NO:55). Consistent with previous observations in vitro and PD-1 data, 5UTR_261 induced a lower immune response. Furthermore, the data indicate that benchmark UTRs designed by others may not be useful in a gene-agnostic manner.

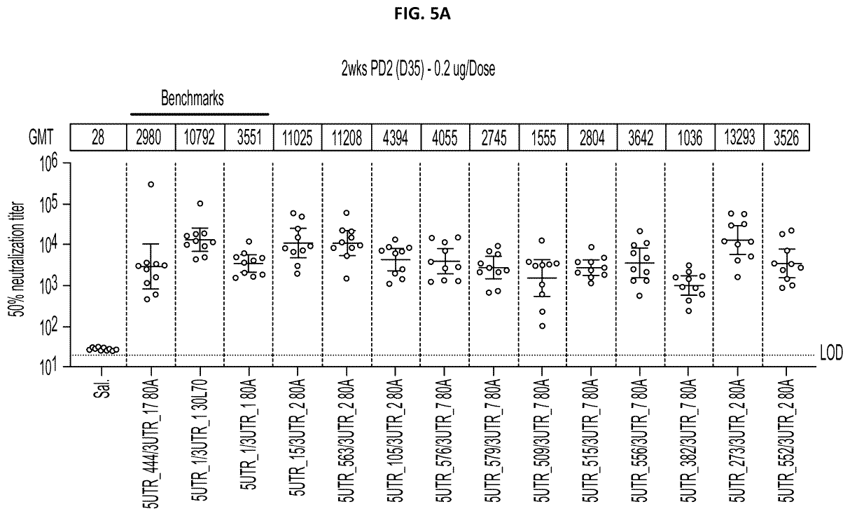

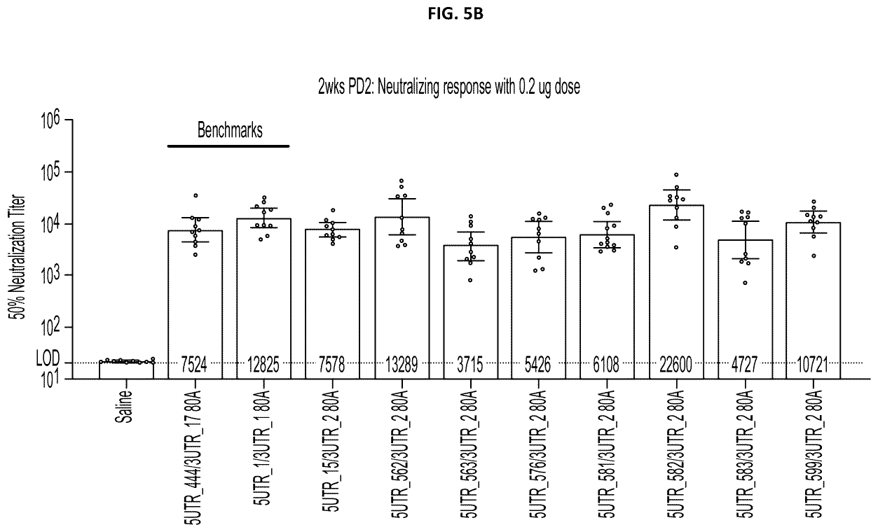

Based on immunogenicity data described in FIG. 3A-3B, several additional novel UTRs were designed and tested for immunogenicity in mice. For example, the seed sequence for 5UTR_562 (SEQ ID NO:86) and 5UTR_563 (SEQ ID NO:87) is 5UTR_15 (SEQ ID NO:50); this parent UTR showed robust immunogenicity in mice using RSV-F as the antigen. Remarkably, 5UTR_563 (SEQ ID NO:87) showed comparable immunogenicity to its parent 5UTR_15 (SEQ ID NO: 50) (FIG. 5A) and 5UTR_562 was the second-best performer in an additional follow-up immunogenicity study in mice (FIG. 5B, below). The RSV 50% geometric mean neutralizing titers for 5UTR_563 (FIG. 5A) were over 3-fold above 5UTR_1/3UTR_1 benchmark (WHO/WHO-80A) and close to 4-fold higher than the 5UTR_444/3UTR_17 benchmark. UTR construct 5UTR_273 (SEQ ID NO:78) was among the best performers with over 4-fold higher neutralizing titers compared to the WHO/WHO-80A benchmark. The 5UTR_273 UTR has a unique feature in that it contains three upstream start codons in the untranslated region itself, which are all in-frame of the start site downstream of the Kozak sequence. The 5UTR_105 sequence has one such upstream in-frame start codon. However, initiation of translation at an alternate upstream start site may add unwanted amino acids to the protein terminus. Therefore, UTR variants based on 5UTR_105 and 5UTR_273 were designed, in which these upstream start codons were removed. A subsequent mouse study (FIG. 5B) examined additional UTR variants (including 5UTR_576 based on 5UTR_105, and 5UTR_581/582/583/599 based on 5UTR_273). In this study, notably, RSV-F modRNA-LNP based on 5UTR_582 induced robust neutralizing responses higher than the 5UTR_1/3UTR_1 benchmark as well as other UTRs.

These examples also demonstrate that minimizing secondary structures in the 5′ UTR, can drive robust expression of the encoded gene both in vitro and in vivo. For example, the activity (data not shown) of the 5UTR_15 (SEQ ID NO:50) was comparable to benchmark sequences and also to a previously published UTR sequence (NCA7d/TPRKB-80A), which was engineered for high expression ((Zeng et al., Adv Mater 32 (40): e2004452 [2020]). (The NCA7d sequence used herein, 5UTR_5, is modified from Table S2 of Zeng to use a GCCACC Kozak motif, and to contain a 5′ end suitable for cleancap reagent). This UTR also robustly expressed RSV-F protein in vitro and elicited strong RSV immune responses in mice. Variants of 5UTR_15 (including 5UTR_562 (SEQ ID NO: 86) and 5UTR_563 (SEQ ID NO:87)) that were also designed to minimize secondary structure also gave robust RSV neutralizing responses in mice (FIG. 5B, see 5UTR_562). Together, these results suggest that rational designs may generate UTRs that can drive expression of multiple genes of interest that yield potent immune responses to the target protein of interest encoded from the mRNA.

### In Vitro Protocols

In vitro Expression (IVE) for the modRNA LNP drug products was assessed by transfecting HEK293F cells with a dose response curves and staining for antibodies, RSV mAb1 specific for the trimeric RSV prefusion F-protein and L4-6 specific for total RSV F-protein. These antibodies have been shown to recognize both the A-strain and B-strain RSV F-protein and were used in the assay with either permeabilizing or non-permeabilizing conditions to assess the total cell vs cell surface content of the RSV F-protein. The description of the protocol below is for total staining (surface+intracellular proteins), for surface only staining protocol, the fixation and wash buffers are changed to remove the permeabilization agent and all other steps and reagents are the same. In brief, 96-well culture plates were seeded with HEK293F cells at a density of 2.5×10⁵ cells/well and were placed in a shaking incubator (350 RPM, 37° C., humidified, 8% CO2) while the samples titrations were prepared. The LNP drug product was diluted in DPBS to a concentration of 160 ng/uL and serially diluted 11-points with a 2.5 dilution factor. After the sample preparation was completed, the 96-well culture plate was removed from the incubator and 50 uL of each point of the diluted LNP were added to duplicate wells of the 96-well culture plate to generate a titration curve ranging from 4,000 ng/well-0.42 ng/well. The 96-well culture plate was placed back into the shaking incubator overnight. After incubation, 250 uL of cells are transferred to a 96-well u-bottom polystyrene plate and pelleted using a swinging bucket centrifuge at 500 radial centrifugal force (rcf) for 5 minutes (min) at room temperature (RT). The supernatant is aspirated, and cells resuspended in 100 uL solution of Aqua405 live/dead stain [Invitrogen, cat #L34966]. The plate is incubated 15 to 30 min at room temperature, protected from light. After incubation, cells are washed with wash buffer [BD, cat #51-2091KZ] and pelleted using swinging bucket centrifuge at 500 rcf for 5 min at RT. The supernatant is aspirated, cells are resuspended in 100 uL fixation/permeabilization buffer [BD, cat #51-2091KZ] and the plate is incubated 30 minutes at 2-8° C., protected from light. Once incubation is complete, the cells are pelleted using a swinging bucket centrifuge at 500 rcf for 5 min at RT. The supernatant is aspirated, and cells resuspended with 250 uL wash buffer, the washing of the cell pellet is repeated twice. After the final wash step, the cells are pelleted, supernatant aspirated, and resuspended in 50 uL of primary antibody solution. The plates are sealed and incubated for 45 minutes at 2-8° C., protected from light. Once incubation is completed, the cells are pelleted using a swinging bucket centrifuge at 500 rcf for 5 min at RT. The supernatant is aspirated, and cells resuspended with 250 uL wash buffer, the washing of the cell pellet is repeated twice. After the final wash step, the cells are pelleted, supernatant aspirated, and resuspended in 50 uL of secondary antibody solution. The plates are sealed and incubated for 45 minutes at 2-8° C., protected from light. Once the incubation is completed the cells are pelleted using a swinging bucket centrifuge at 500 rcf for 5 min at RT. The supernatant is removed and cells resuspended with 250 uL wash buffer, this is repeated twice. After the final wash step cells are pelleted, supernatant removed, and resuspended in 200 uL of wash buffer and data acquired by flow cytometry.

### In Vivo Protocol (Mouse Study)

Female BALB/c mice were immunized with RSV prefusion F (847) in bivalent protein subunit version (RSV 847A+847B) as described in WO2017/109629 or modRNA-LNP formulation described herein either as monovalent (RSV 847A) or bivalent (RSV 847A+847B) at different doses on day 0 and day 21. Immunogenicity was evaluated by measuring RSV neutralizing antibody response. Serum was collected on day 21 and day 35 (2 weeks post dose 2, PD2) in a RSV neutralization assay (see below).

### RSV Neutralization Assay

The RSV microneutralization assay is a 3-day assay using A549 cells (human alveolar basal epithelial cells) to measure functional antibodies in serum that neutralize RSV activity, preventing infection of a host cell monolayer. On Day 0, A549 cells (human alveolar basal epithelial cells; ATCC, cat #CCL-185) are seeded in 96-well tissue-culture treated plates at 2.5×10⁴ cells per well and incubated for at least 20 hours to form a confluent monolayer. On Day 1, diluted virus (RSV A, M37; RSV B, B18537; 500 FFU/well) is added to 3-fold serial dilutions of heat inactivated test serum prepared in duplicate and incubated for 1 hour to allow antibodies to bind to the virus. The neutralization reaction is then transferred onto the prepared A549 cell monolayers and incubated for 2 hours. Additional media is supplemented onto the plates prior to an overnight incubation (at least 16 hours). On Day 2, the plates are fixed with methanol and stained with a human anti-RSV F (Sobi, Synagis) primary antibody followed by an Alexa 488 fluorescently labeled secondary antibody to detect viral foci. A 50% neutralization titer is calculated as the last reciprocal serum dilution at which 50% of the virus is neutralized compared to wells containing virus only. A titer is reported as the geometric mean titer (GMT) of the two replicate titers of each sample. The assay titer range is 20 to 43,740. Any samples with a titer >43,740 are prediluted and repeated to extend the upper titer limit. Any samples below the lower limit of detection (LLOD) are reported at LLOD of 20.

### Example 4: Evaluation of Different UTR Designs in modRNA Encoding Full-Length hMPV F Protein in Mice

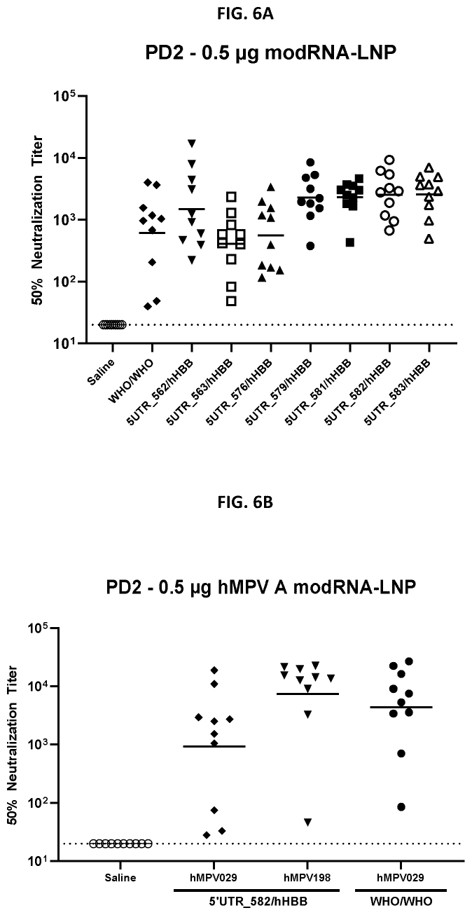

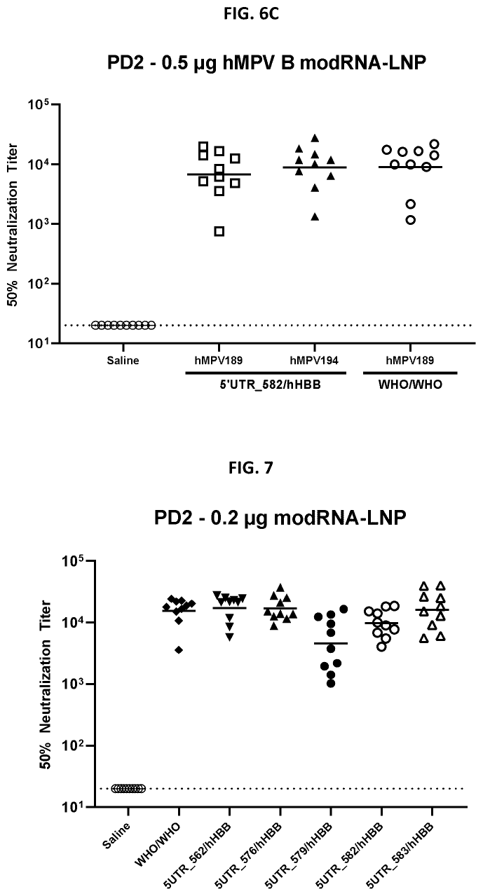

Formulated modRNA-LNPs were prepared as described in Examples 1 and 2 above. One lower dose level was selected in order to provide more sensitive results and potentially differentiate different UTR designs. Female Balb/c mice were immunized with 0.5 μg of LNP-formulated modRNA encoding tested UTR designs in combination with full-length hMPV A F mutant hMPV021 as the gene of interest (GOI). Immunizations were given intramuscularly at weeks 0 and 3 (Table 41). Post-dose 1 (PD1, week 3) and post-dose 2 (PD2, week 5) sera were evaluated in an hMPV neutralization assay described below. Briefly, neutralizing titers were determined as the serum dilution factor resulting in a 50% reduction in infectious units. Results are reported as the geometric mean titer from 10 mice per group. Fold rise in 50% geometric mean titers are reported as the ratio of post-dose 2 (PD2) of the novel UTR designs to the WHO/WHO benchmark within each group (Table 42). Overall, all UTR designs tested elicited a neutralizing response following two immunizations in mice compared with the saline reference (FIG. 6A). Among the novel UTR designs, 5UTR_579/hHBB, 5UTR_581/hHBB, 5UTR_582/hHBB, and 5UTR_583/hHBB showed at least 3-fold higher neutralizing GMTs than the benchmark UTR, suggesting that these UTR designs are more immunogenic than the benchmark UTR.

TABLE 41
Immunization schedule of the murine immunogenicity
study comparing novel UTR designs in combination
with full-length hMPV A F encoded from modRNA.

| hMPV A F protein modRNA dose | 0.5 μg modRNA-LNP |
|---|---|
| Vaccination | Weeks 0, 3 |
| Bleed | Week 3 (PD1) |
| | Week 5 (PD2) |

TABLE 42
Geometric mean and fold-rise of 50% neutralizing
titers of Balb/c mice following two immunizations
with modRNA-LNP of hMPV A F protein.

0.5 μg modRNA-LNP

| UTR-Design | GMT | Fold rise |
|---|---|---|
| WHO/WHO | 609 | 1.0 |
| 5UTR_562/hHBB | 1474 | 2.4 |
| 5UTR_563/hHBB | 409 | 0.7 |
| 5UTR_576/hHBB | 558 | 0.9 |
| 5UTR_579/hHBB | 2264 | 3.7 |
| 5UTR_581/hHBB | 2299 | 3.8 |
| 5UTR_582/hHBB | 2536 | 4.2 |
| 5UTR_583/hHBB | 2587 | 4.2 |

hMPV Neutralization Assay

The hMPV microneutralization assay quantitatively measures functional antibodies in serum that neutralize hMPV activity, preventing infection of a host cell monolayer. On Day 0, A549 cells (human alveolar basal epithelial cells; ATCC, cat #CCL-185) are seeded in 96-well tissue-culture treated plates at 2.5×10\hat{ }4 cells per well and incubated for 16-24 hours to form a confluent monolayer. On Day 1, diluted virus (hMPV A, GFP CAN97-83; hMPV B, TN/83-1211; 500 FFU/well) is added to 3-fold serial dilutions of heat inactivated test serum prepared in duplicate and incubated for 1 hour at 37° C./5% CO2 to allow antibodies to bind to the virus. The neutralization reaction is then transferred onto the prepared A549 cell monolayers. On Day 2, plates are fixed after 22-26 hours of infection at 37° C./5% CO2. hMPV A plates are fixed with 4% PFA and infected cells are detected by GFP fluorescence. hMPV B plates are fixed with methanol and stained with a chimeric rabbit Fc/human Fab anti-hMPV F primary antibody followed by an Alexa 488 fluorescently labeled secondary antibody to detect viral foci. Fluorescently labeled infected cells are quantified using a CTL-ImmunoSpot Analyzer. A 50% neutralization titer is calculated as the last reciprocal serum dilution at which 50% of the virus is neutralized compared to wells containing virus only. A titer is reported as the geometric mean titer (GMT) of the two replicate titers of each sample. The assay titer range is 20 to 43,740. Any samples with a titer >43,740 are prediluted and repeated to extend the upper titer limit. Any samples below the lower limit of detection (LLOD) are reported at LLOD of 20.

### Example 5: Evaluation of Different UTR Designs with modRNA Encoding Full-Length PIV1 F Protein in Mice

Formulated modRNA-LNPs were prepared as described in Examples 1 and 2 above. One lower dose level was selected in order to provide more sensitive results and potentially differentiate different UTR designs. Female Balb/c mice were immunized with 0.2 μg of LNP-formulated modRNA encoding tested UTR designs in combination with full-length PIV1 F protein mutant PIV1047 as gene of interest (GOI). Immunizations were given intramuscularly at weeks 0 and 3 (Table 43). Post-dose 1 (PD1, week 3) and post-dose 2 (PD2, week 5) sera were evaluated in an PIV1 neutralization assay as described below. Briefly, neutralizing titers were determined as the serum dilution factor resulting in a 50% reduction in infectious units. Results are reported as the geometric mean titer from 10 mice per group. Fold rise in 50% geometric mean titers are reported as the ratio of post-dose 2 (PD2) of the novel UTR designs to the WHO/WHO benchmark within each group (Table 44). Overall, all UTR designs tested elicited a neutralizing response following two immunizations in mice compared with the saline reference (FIG. 7). Among the novel UTR designs, 5UTR_562/hHBB, 5UTR_576/hHBB, and 5UTR_583/hHBB showed comparable immunogenicity to the benchmark UTR, with the first two UTRs demonstrating highest neutralizing antibody responses.

TABLE 43
Immunization schedule of the murine immunogenicity
study comparing novel UTR designs in combination
with full-length PIV1 F encoded from modRNA.

| PIV1 F protein modRNA dose | 0.2 μg modRNA-LNP |
|---|---|
| Vaccination | Weeks 0, 3 |
| Bleed | Week 3 (PD1) |
| | Week 5 (PD2) |

TABLE 44
Geometric mean and fold-rise of 50% neutralizing
titers of Balb/c mice following two immunizations
(PD2) with modRNA-LNP of PIV1 F protein.

0.2 μg modRNA-LNP

| UTR-Design | GMT | Fold rise |
|---|---|---|
| WHO/WHO | 15462 | 1.0 |
| 5UTR_562/hHBB | 17184 | 1.1 |
| 5UTR_576/hHBB | 17011 | 1.1 |
| 5UTR_579/hHBB | 4580 | 0.3 |
| 5UTR_582/hHBB | 9728 | 0.6 |
| 5UTR_583/hHBB | 16100 | 1.0 |

### PIV1 Neutralization Assay

The PIV1 microneutralization assay quantitatively measures functional antibodies in serum that neutralize PIV1 activity, preventing infection of a host cell monolayer. On Day 0, A549 cells (human alveolar basal epithelial cells; ATCC, cat #CCL-185) are seeded in 96-well tissue-culture treated plates at 2.5×10\hat{ }4 cells per well and incubated for 16-24 hours to form a confluent monolayer. On Day 1, diluted virus (PIV1-GFP WA/20993/1964 Strain; 500 FFU/well) is added to serial dilutions of heat inactivated test serum prepared in duplicate and incubated for 1 hour at 37° C./5% CO2 to allow antibodies to bind to the virus. The neutralization reaction is then transferred onto the prepared A549 cell monolayers. On Day 2, plates are fixed after 22-26 hours of infection at 37° C./5% CO2. PIV1 plates are fixed with 4% PFA and infected cells are detected by GFP fluorescence. Fluorescently labeled infected cells are quantified using a CTL-ImmunoSpot Analyzer. A 50% neutralization titer is calculated as the last reciprocal serum dilution at which 50% of the virus is neutralized compared to wells containing virus only. A titer is reported as the geometric mean titer (GMT) of the two replicate titers of each sample. The assay titer range is 20 to 43,740. Any samples with a titer >43,740 are prediluted and repeated to extend the upper titer limit. Any samples below the lower limit of detection (LLOD) are reported at LLOD of 20.

### Example 6: Evaluation of Different UTR Designs with modRNA Encoding Full-Length PIV3 F Protein in Mice

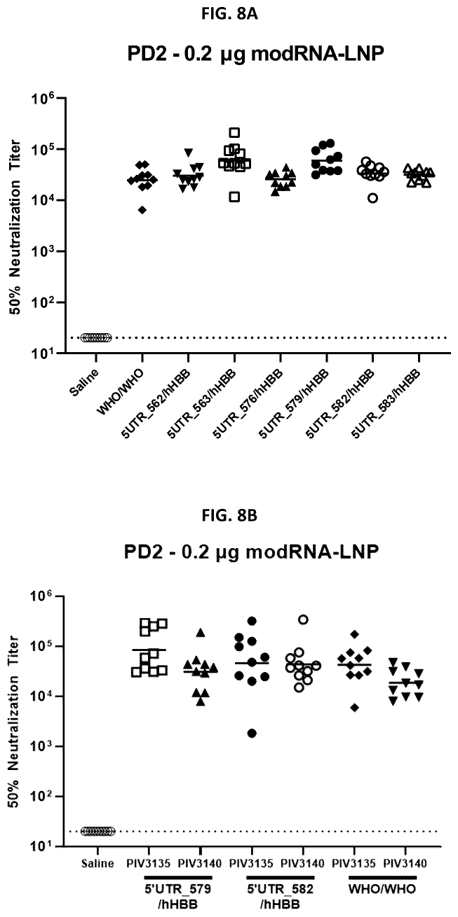

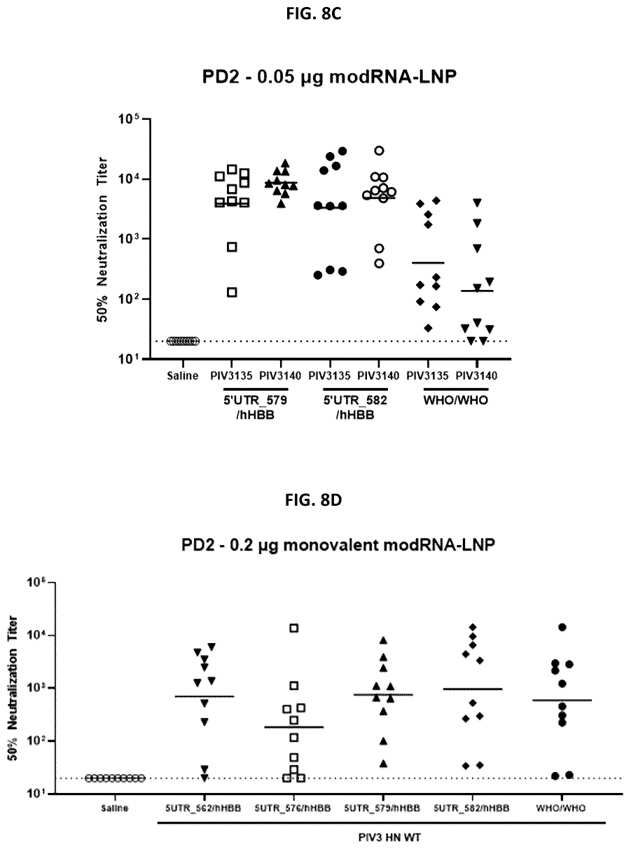

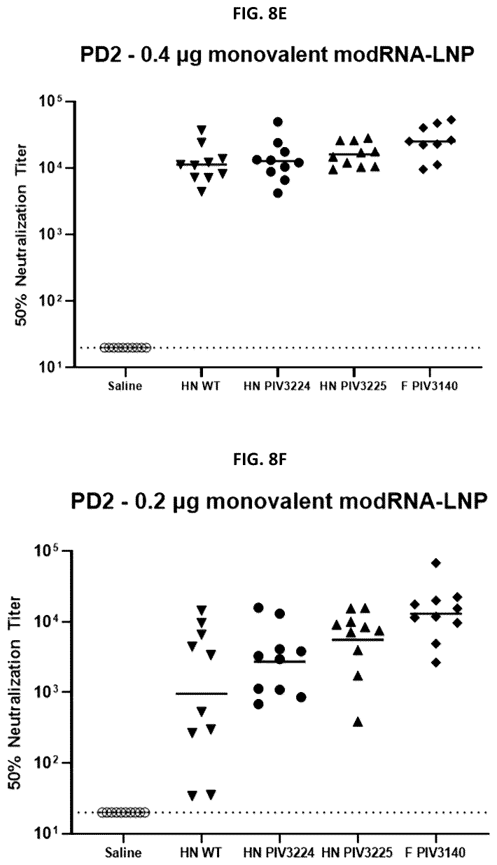

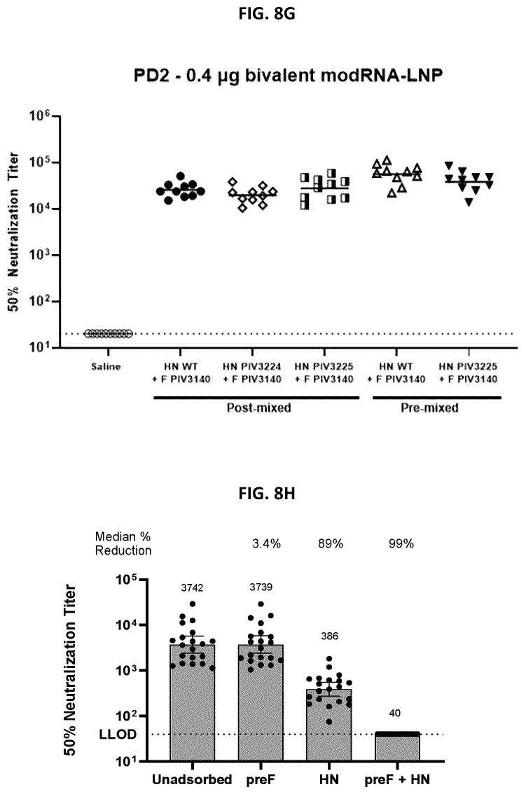

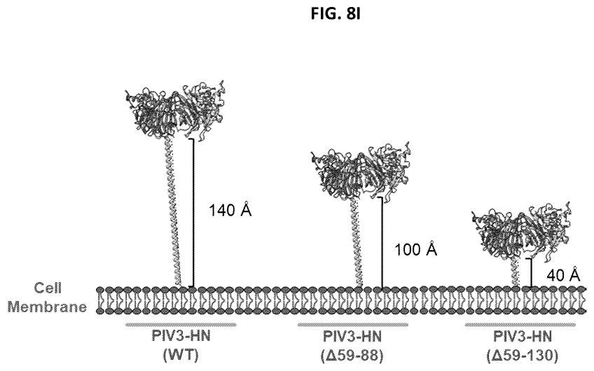

Formulated modRNA-LNPs were prepared as described in Examples 1 and 2 above. One lower dose level was selected in order to provide more sensitive results and potentially differentiate different UTR designs. Female Balb/c mice were immunized with 0.2 μg of LNP-formulated modRNA encoding tested UTR designs in combination with full-length PIV3 F protein mutant PIV3008 as gene of interest (GOI). Immunizations were given intramuscularly at weeks 0 and 3 (Table 45). Post-dose 1 (PD1, week 3) and post-dose 2 (PD2, week 5) sera were evaluated in an PIV3 neutralization assay as described below. Briefly, neutralizing titers were determined as the serum dilution factor resulting in a 50% reduction in infectious units. Results are reported as the geometric mean titer from 10 mice per group. Fold rise in 50% geometric mean titers are reported as the ratio of post-dose 2 (PD2) of the novel UTR designs to the WHO/WHO benchmark UTR within each group (Table 46). Overall, all UTR designs tested elicited a neutralizing response following two immunizations in mice compared with the saline reference (FIG. 8A). All novel UTR designs showed better or comparable neutralizing GMTs to the benchmark UTR.

TABLE 45
Immunization schedule of the murine immunogenicity
study comparing novel UTR designs in combination with
full-length PIV3 F protein encoded from modRNA.

| PIV3 F protein modRNA dose | 0.2 μg modRNA-LNP |
|---|---|
| Vaccination | Weeks 0, 3 |
| Bleed | Week 3 (PD1) |
| | Week 5 (PD2) |

TABLE 46
Geometric mean and fold-rise of 50% neutralizing
titers of Balb/c mice following two immunizations
(PD2) with modRNA-LNP of PIV3 F protein.

0.2 μg modRNA-
LNP

| UTR-Design | GMT | Fold rise |
|---|---|---|
| WHO/WHO | 24512 | 1.0 |
| 5UTR_562/hHBB | 30036 | 1.2 |
| 5UTR_563/hHBB | 59646 | 2.4 |
| 5UTR_576/hHBB | 25582 | 1.0 |
| 5UTR_579/hHBB | 59285 | 2.4 |
| 5UTR_582/hHBB | 33067 | 1.3 |
| 5UTR_583/hHBB | 31593 | 1.3 |

### PIV3 Neutralization Assay

The PIV3 microneutralization assay quantitatively measures functional antibodies in serum that neutralize PIV3 activity, preventing infection of a host cell monolayer. On Day 0, A549 cells (human alveolar basal epithelial cells; ATCC, cat #CCL-185) are seeded in 384-well tissue-culture treated plates at 1.25×10\hat{ }4 cells per well and incubated for 16-24 hours to form a confluent monolayer. On Day 1, diluted virus (PIV3-GFP P1 JS Strain; 500 FFU/well) is added to serial dilutions of heat inactivated test serum prepared in duplicate and incubated for 45 min at 37° C./5% CO2 to allow antibodies to bind to the virus. The neutralization reaction is then transferred onto the prepared A549 cell monolayers. On Day 2, plates are fixed after 24 hours of infection at 34° C./5% CO2. PIV3 plates are fixed with 4% PFA and infected cells are detected by GFP fluorescence. Fluorescently labeled infected cells are quantified using a CTL-ImmunoSpot Analyzer. A 50% neutralization titer is calculated as the last reciprocal serum dilution at which 50% of the virus is neutralized compared to wells containing virus only. A titer is reported as the geometric mean titer (GMT) of the two replicate titers of each sample. The assay titer range is 20 to 43,740. Any samples with a titer >43,740 are prediluted and repeated to extend the upper titer limit. Any samples below the lower limit of detection (LLOD) are reported at LLOD of 20.

### Example 7: Design and Selection of modRNA Encoding Full-Length PIV3 F and/or PIV3 HN Proteins

A. Selection of PIV3 preF and/or HN as Vaccine Target Antigens

The PIV3 F protein has been a long-standing target of prophylactic vaccine development based on its role of facilitating the fusion of viral and host-cell membranes. Potent neutralizing antibodies targeting the PIV3 F protein have been reported (Boonyaratanakornkit et al. 2021; Stewart-Jones et al. 2018) and are demonstrated in this Example hereinbelow, which further support vaccine development efforts targeting PIV3 F protein. Unlike RSV and hMPV in the pneumovirinae sub-family, viruses in the paramyxovirineae sub-family utilize HN protein as the host-receptor binding protein. HN has been demonstrated to be a highly immunogenic surface glycoprotein and can also elicit neutralizing antibodies (Henrickson and Portner 1990, Miller et al. 2024, Suryadevara et al. 2024, van Wyke Coelingh et al. 1985). HN-binding antibodies have been reported as the predominant antibody population after natural infection in young children.

To better understand the distribution of neutralizing antibodies post-natural infection, a panel of 20 adult seropositive sera were evaluated for neutralization against PIV3 with or without depletion against purified PIV3 preF, PIV3 HN, or a 1:1 combination of preF and HN antigens. Human adult donor sera (n=20) were pre-adsorbed with 10 μg/mL of stabilized His-tagged prefusion F (Stewart-Jones et al. 2018), HN head domain, or their combination. Antibodies bound to the proteins were removed using nickel beads, and sera from unadsorbed and adsorbed samples were tested in a PIV3 neutralization assay.

The results suggest neutralizing antibodies against PIV3 HN is the predominant population of the neutralizing response after natural infection (FIG. 8H). Depletion of antibodies against preF and HN completely abolished any detectable neutralizing activities of the human sera. Using human sera collected after natural infection as a reference suggests the combination of PIV3 preF and HN antigens would provide the most optimal protection against PIV3 infection and both antigens should be considered as PIV3 vaccine targets.

B. PIV3 F modRNA UTR Selection

The sequences and modRNA constructs of various PIV3 F proteins, including PIV3 F PIV3135 and PIV3 F PIV3140, comprising the WHO/WHO benchmark 5′UTR/3′UTR are described in Examples 27, 29, 30 and 31 of WO2024/154048, which is hereby incorporated by reference herein in its entirety. In the mouse study described in Example 31 of WO2024/154048 the various PIV3 F protein modRNA constructs comprising the WHO/WHO benchmark UTRs were evaluated for immunogenicity. BALB/c mice were immunized twice with modRNAs carrying different PIV3 preF variants at 0.2 μg dose level. The modRNA encoding the full-length WT was included for comparison. Immunizations were given intramuscularly at weeks 0 and 3. Post-dose 2 (PD2, week 5) sera were evaluated in a PIV3 neutralization assay. Results are reported as the geometric mean titer from 10 mice per group of post-dose 2 (PD2). Both PIV3135 and PIV3140 variants demonstrated approximately 8-fold higher neutralizing titers as compared to the WT construct (Table 47).

TABLE 47
Geometric mean of 50% neutralizing titers of BALB/c mice following
two immunizations (PD2) with modRNA-LNP of PIV3 F mutants

| Mutant ID | GMT |
|---|---|
| WT | 3172 |
| PIV3031 | 3694 |
| PIV3135 | 27695 |
| PIV3138 | 5600 |
| PIV3140 | 24683 |
| PIV3141 | 9881 |
| PIV3165 | 9249 |
| PIV3167 | 14792 |
| Saline | 20 |

The follow up study set forth herein evaluated the immunogenicity of modRNA constructs with selected combination of lead UTRs and PIV3 preF designs in mice. Formulated modRNA-LNPs were prepared as described in Examples 1 and 2 above. In this study, novel 5′UTR/3′UTR designs 5UTR_579/hHBB and 5UTR_582/hHBB, with either full-length PIV3 F PIV3135 or PIV3 F PIV3140 as the gene of interest (GOI) were tested. Two dose levels were selected to aid differentiation of immune responses among the different UTR designs. Female BALB/c mice were immunized with 0.05 μg or 0.2 μg of LNP-formulated modRNA encoding either WHO/WHO benchmark 5′UTR/3′UTR or the novel 5′UTR/3′UTR designs 5UTR_579/hHBB and 5UTR_582/hHBB, with either full-length PIV3 F PIV3135 or PIV3 F PIV3140 as the gene of interest (GOI). Immunizations were given intramuscularly at weeks 0 and 3 (Table 48). Post-dose 2 (PD2, week 5) sera were evaluated in a PIV3 neutralization assay as described in Example 3 with minor modifications. Results are reported as the geometric mean titer from 10 mice per group of post-dose 2 (PD2) (Table 49).

TABLE 48
Immunization schedule of the murine immunogenicity
study comparing UTR designs in combination with full-
length PIV3 F top candidates encoded from modRNA.

| | |
|---|---|
| PIV3 F protein modRNA dose | 0.05 μg or 0.2 μg modRNA-LNP |
| Vaccination | Weeks 0, 3 |
| Bleed | Week 5 (PD2) |

TABLE 49
Geometric mean of 50% neutralizing titers of BALB/c mice following
two immunizations (PD2) with modRNA-LNP of PIV3 F mutants

| PIV3 F | | 0.2 μg modRNA-LNP | 0.05 μg modRNA-LNP |
|---|---|---|---|
| Mutant | UTR Design | GMT | GMT |
| PIV3135 | WHO/WHO | 42303 | 401 |
| PIV3140 | WHO/WHO | 18527 | 137 |
| PIV3135 | 5UTR_579/hHBB | 84313 | 3877 |
| PIV3140 | 5UTR_579/hHBB | 30607 | 8678 |
| PIV3135 | 5UTR_582/hHBB | 45660 | 3312 |
| PIV3140 | 5UTR_582/hHBB | 43409 | 4813 |
| Saline | N/A | 20 | 20 |

Overall, all PIV3 F mutants elicited neutralizing responses following two immunizations in mice compared with the saline group (Table 49, FIG. 8B and FIG. 8C). PIV3135 and PIV3140 modRNAs containing 5UTR_579/hHBB and 5UTR_582/hHBB novel UTRs elicited robust and comparable PIV3 neutralizing antibody responses at both the 0.05 μg and 0.2 μg dose levels. While the neutralizing responses of the PIV3 modRNAs with novel UTRs and WHO/WHO benchmark UTR were comparable at 0.2 μg dose level, the differences became more apparent at the 0.05 μg dose. For instance, at the low dose of 0.05 μg, PIV3135 and PIV3140 containing 5UTR_579/hHBB demonstrated a 9.6-fold and 62.7-fold higher neutralizing antibody responses when compared to their corresponding WHO/WHO design (FIG. 8C). The mouse immunogenicity data supports the use of PIV3 F mutant PIV3135 and PIV3140 modRNAs in combination with 5UTR_579/hHBB or 5UTR_582/hHBB as PIV3 vaccine candidates.

C. PIV3 HN modRNA

PIV3 HN is a 572 amino acid type II membrane protein that exists as a covalently linked dimer composed of a N-terminal intravirion region or intracellular domain (amino acids 1 to 31), a transmembrane domain (amino acids 32 to 52), and an ectodomain comprising a stalk region (amino acids 53 to 146) and a globular head region (amino acids 147 to 572), which carries an active site forsialic acid binding and cleavage (Lawrence et al. 2004, Marcink et al. 2020, Marcink et al. 2023, Moscona 2005). The PIV3 HN proteins are well conserved and an example sequence of the PIV3 HN portion is provided in SEQ ID NO: 724 (Strain: HPIV3/MEX/2545/2006, accession number: AGT75286). SEQ ID NO:724 is a 572 amino acid sequence.

It has been observed that by forming a complex with the F protein on the virion surface, HN stabilizes the preF prior to receptor engagement and induces F conformational change for cell membrane fusion upon host cell attachment (Chang and Dutch 2012). One of the dimeric globular heads of HN has been observed to cap the apex of preF through an extended loop interaction, forming an outer HN layer followed by an inner preF layer on the PIV3 virion (Marcink et al. 2020, Marcink et al. 2023).

Considering the architecture of HN-preF complex and its display on the virion surface, the position and the long stalk region of the HN protein could potentially hinder the display of both preF and HN when they are expressed as membrane-bound antigens through LNP-formulated modRNAs. Accordingly, two HN variants containing varying truncations (e.g. deletions) at the stalk region at amino acid residues 59-88 and 59-130 (designated A59-88 and A59-130, respectively) were designed (FIG. 8I) and compared against the wild-type HN in a mouse immunogenicity study (Table 51).

This mouse study evaluated two key objectives: 1) the immunogenicity of PIV3 HN in combination with novel UTRs as compared to a benchmark UTR (WHO/WHO), and 2) the immunogenicity of PIV3 HN mutant designs as compared with WT. Formulated modRNA-LNPs were prepared as described in Example 1 and 2 above. Female BALB/c mice (N=10) were immunized with 0.2 μg or 0.4 μg of LNP-formulated modRNA encoding either PIV3 HN WT, mutant PIV3224 or PIV3225, expressed with WHO/WHO benchmark UTR or novel UTR pairs (e.g. 5UTR_562/hHBB, 5UTR_576/hHBB, 5UTR_579/hHBB and 5UTR_582/hHBB). Immunizations were given intramuscularly at weeks 0 and 3 (Table 50). Post-dose 2 (PD2, week 5) sera were evaluated in an PIV3 neutralization assay as described in Example 3 with minor modifications. Results are reported as the geometric mean titer from 10 mice per group of post-dose 2 (PD2) (Table 51).

TABLE 50
Immunization schedule of the murine immunogenicity
study comparing PIV3 HN and F encoded from modRNA.

| | |
|---|---|
| PIV3 F and HN modRNA dose | 0.2 μg, 0.4 μg modRNA-LNP |
| Vaccination | Weeks 0, 3 |
| Bleed | Week 5 (PD2) |

TABLE 51
Geometric mean of 50% neutralizing titers of BALB/c
mice following two immunizations (PD2) with modRNA-
LNP comprising PIV3 F and HN alone or combined.

| | | 0.4 μg | 0.2 μg |
|---|---|---|---|
| PIV3 F | | modRNA- | modRNA- |
| and HN | | LNP | LNP |
| designs | UTR Design | GMT | GMT |
| PIV3 HN WT | WHO/WHO | N/A | 588 |
| PIV3 HN WT | 5UTR_562/hHBB | N/A | 700 |
| PIV3 HN WT | 5UTR_576/hHBB | N/A | 183 |
| PIV3 HN WT | 5UTR_579/hHBB | N/A | 750 |
| PIV3 HN WT | 5UTR_582/hHBB | 11281 | 959 |
| PIV3 HN | 5UTR_582/hHBB | 12723 | 2723 |
| PIV3224 | | | |
| (Δ59-88) | | | |
| PIV3 HN | 5UTR_582/hHBB | 15985 | 5529 |
| PIV3225 | | | |
| (Δ59-130) | | | |
| Saline | N/A | 20 | 20 |

“Δ” denotes deletion of amino acids at indicated positions of a polypeptide

Overall, all PIV3 HN modRNAs elicited a neutralizing response following two immunizations in mice compared with the saline reference (Table 51, FIG. 8D-8G). For monovalent RNA-LNP at the low 0.2 μg dosage, compared to WHO/WHO benchmark UTR, all PIV3 HN WT expressed through novel UTRs elicited comparable neutralizing antibody titers, except for 5UTR_576/hHBB which showed 3.2-fold lower neutralizing responses (FIG. 8D). This mouse immunogenicity data supports the use of novel UTRs with PIV3 HN.

While the neutralizing responses of PIV3 HN WT and mutants were similar at 0.4 μg dose level, the differences became more pronounced at the 0.2 μg dose (Table 51, FIG. 8E). At 0.2 μg dose level, the most truncated variant HN mutant PIV3225 (A59-130) elicited 5.8-fold and 2.0-fold higher neutralizing titers than WT and PIV3224 (A59-88), respectively (FIG. 8F). This suggests that PIV3225 was the more immunogenic form of PIV3 HN at the lower dosage.

### Example 8: Design and Selection of modRNA Encoding Full-Length hMPV F Proteins

A. Evaluation of Selected UTR Designs with modRNA Encoding Full-Length hMPV F Protein in Mice

This study evaluated the immunogenicity of modRNA constructs with selected combination of lead UTRs and hMPV A/B preF design in mice. Formulated modRNA-LNPs were prepared as described in Examples 1 and 2 above. One lower dose level was selected in order to provide more sensitive results and potentially differentiate different UTR designs. Female BALB/c mice were immunized with 0.5 μg of LNP-formulated modRNA encoding either WHO/WHO benchmark UTR or 5UTR_582/hHBB, with full-length hMPV A F protein mutant hMPV029 or hMPV198, or hMPV B F protein mutant hMPV189 or hMPV194 as the gene of interest (GOI). Immunizations were given intramuscularly at weeks 0 and 3 (Table 52). Post-dose 2 (PD2, week 5) sera were evaluated in hMPV A or hMPV B neutralization assay as described in Example 3 with minor. Results are reported as the geometric mean titer from 10 mice per group of post-dose 2 (PD2) (Table 53 and 54).

TABLE 52
Immunization schedule of the murine immunogenicity
study comparing UTR designs in combination with full-
length hMPV F top candidates encoded from modRNA.

| | |
|---|---|
| hMPV F protein modRNA dose | 0.5 μg modRNA-LNP |
| Vaccination | Weeks 0, 3 |
| Bleed | Week 5 (PD2) |

TABLE 53
Geometric mean of 50% neutralizing titers against
hMPV A of BALB/c mice following two immunizations
(PD2) with modRNA-LNP of hMPV A F mutants.

| | | 0.5 μg modRNA-LNP |
|---|---|---|
| hMPV F Mutant | UTR Design | GMT |
| hMPV029 A | WHO/WHO | 4383 |
| hMPV029 A | 5UTR_582/hHBB | 928 |
| hMPV198 A | 5UTR_582/hHBB | 7414 |
| Saline | N/A | 20 |

TABLE 54
Geometric mean of 50% neutralizing titers against
hMPV B of BALB/c mice following two immunizations
(PD2) with modRNA-LNP of hMPV B F mutants.

| | | 0.5 μg modRNA-LNP |
|---|---|---|
| hMPV F Mutant | UTR Design | GMT |
| hMPV189 B | WHO/WHO | 8894 |
| hMPV189 B | 5UTR_582/hHBB | 6740 |
| hMPV194 B | 5UTR_582/hHBB | 8781 |
| Saline | N/A | 20 |

Overall, all hMPV F mutants elicited a neutralizing response following two immunizations in mice (Table 53 and 54, FIG. 6B and FIG. 6C). For hMPV A F mutant hMPV198 encoded through 5UTR_582/hHBB showed 1.7-fold and 8-fold higher neutralizing responses than hMPV029 expressed through 5UTR_582/hHBB and WHO/WHO, respectively (FIG. 6B). For hMPV B, both F mutants (hMPV189 and hMPV194) expressed through 5UTR_582/hHBB showed similar neutralizing antibody responses when compared with hMPV189 expressed through WHO/WHO benchmark UTR (FIG. 6C). The mouse immunogenicity data support the use of 5UTR_582/hHBB and the hMPV preF designs as hMPV vaccine candidates.

### Example 9: Antigen Sequences

Sequences of the antigens/polypeptides, DNA and RNA of the present invention are provided in Tables 1-18. The sequences may comprise any stop codon, including but not limited to the stop codons provided in the Tables below.

### A. RSV Sequences

TABLE 1
RSV F Polypeptides

| ID | SEQ ID NO: |
|---|---|
| Full Length F0 of Native RSV A2 (GenBank GI: | 1 |
| 138251; Swiss Prot P03420) | |
| Full Length F0 of Native RSV B (18537 strain; | 2 |
| GenBank GI: 138250; Swiss Prot P13843) | |
| RSV A WT F | 3 |
| RSV F 847A | 4 |
| RSV B WT F | 5 |
| RSV F 847B | 6 |
| RSV F 851A | 621 |
| RSV F 851B | 622 |
| RSV F 852A | 623 |
| RSV F 852B | 624 |
| RSV F 847A Ecto-Foldon | 625 |
| RSV F 847B Ecto-Foldon | 626 |

TABLE 2
RSV F DNA

| | | SEQ |
|---|---|---|
| ID | | ID NO: |
| RSV A WT F | | 7 |
| TGA stop codon; (Amino acid SEQ ID NO: 3) | | |
| RSV F 847A | | 8 |
| TGA stop codon; (Amino acid SEQ ID NO: 4) | | |
| RSV B WT F | | 9 |
| TGA stop codon; (Amino acid SEQ ID NO: 5) | | |
| RSV F 847B | | 10 |
| TGA stop codon; (Amino acid SEQ ID NO: 6) | | |
| RSV F 851A | | 627 |
| TGA stop codon; (Amino acid SEQ ID NO: 621) | | |
| RSV F 851B | | 628 |
| TGA stop codon; (Amino acid SEQ ID NO: 622) | | |
| RSV F 852A | | 629 |
| TGA stop codon; (Amino acid SEQ ID NO: 623) | | |
| RSV F 852B | | 630 |
| TGA stop codon; (Amino acid SEQ ID NO: 624) | | |
| RSV F 847A Ecto-Foldon | | 631 |
| TGA stop codon; (Amino acid SEQ ID NO: 625) | | |
| RSV F 847B Ecto-Foldon | | 632 |
| TGA stop codon; (Amino acid SEQ ID NO: 626) | | |
| | | SEQ |
| ID | RSV Sequence | ID NO: |
| RSV F | AGGAAATAAGAGAGGATAAGACGACTAAGGAGACATAC | 11 |
| 847A_5UTR_563 | AGAATAAGAAACAGGCAGCCACCATGGAACTGCCCATCC | |
| /3UTR_2 80A | TGAAAACAAACGCCATCACCACCATCCTGGCCGCCGTGAC | |
| RSV F 847A | ACTGTGTTTTGCCAGCAGCCAGAACATCACCGAGGAATTC | |
| DNA | TACCAGAGCACCTGTAGCGCCGTGTCCAAGGGCTATCTG | |
| Underline = 5′ | AGCGCCCTTAGAACCGGCTGGTACACCAGCGTGATCACC | |
| cap; bold = | ATCGAGCTGAGCAACATCAAAGAAAACAAGTGCAACGGCA | |
| 5′UTR | CCGACGCCAAAGTGAAGCTGATCAAGCAAGAGCTGGACA | |
| (5UTR_563) and | AGTACAAGAACGCCGTGACCGAACTGCAGCTGCTGATGC | |
| 3′ UTR | AGTCTACCCCTGCCTGCAATAGCAGAGCCAGACGGGAAC | |
| (3UTR_2); | TGCCTAGATTCATGAACTACACCCTGAACAACACCAAGAA | |
| italics = | CACCAACGTGACCCTGAGCAAGAAGCGGAAGCGGAGATT | |
| KOZAK | CCTGGGCTTTCTGCTCGGAGTGGGAAGCGCCTGCGCCTC | |
| sequence; | TGGAATCGCCGTGTCTAAAGTGCTGCACCTGGAAGGCGA | |
| lowercase = | AGTGAACAAGATCAAGAGCGCCCTGCTGAGCACCAACAA | |
| polyA tail | GGCCGTGGTGTCTCTGAGCAATGGCGTGTCCGTGCTGAC | |
| | CATCAAGGTGCTGGACCTGAAGAACTACATCGACAAACAG | |
| | CTGCTGCCCATCGTCAACAAGCAGAGCTGCAGCATCAGC | |
| | AACATCGAGACAGTGATCGAGTTCCAGCAGAAGAACAACC | |
| | GGCTGCTGGAAATCACCCGCGAGTTCTCTGTGAATGCCG | |
| | GCGTGACCACACCTGTGTCCACCTACATGCTGACCAACAG | |
| | CGAGCTGCTGTCCCTGATCAACGACATGCCCATCACCAAC | |
| | GACCAGAAAAAGCTGATGAGCAGCAACGTGCAGATCGTG | |
| | CGGCAGCAGAGCTACTCCATCATGAGCATTATCAAAGAAG | |
| | AGGTGCTGGCCTACGTGGTGCAGCTGCCTCTGTATGGCG | |
| | TGATCGATACCCCTTGCTGGAAGCTGCACACAAGCCCTCT | |
| | GTGCACCACCAACACCAAAGAGGGCTCCAACATCTGCCT | |
| | GACCAGAACCGACAGAGGCTGGTACTGCGATAATGCCGG | |
| | CAGCGTCTCATTCTTCCCACAAGCCGAGACATGCAAGGTG | |
| | CAGAGCAACCGGGTGTTCTGCGACACCATGAACAGCCTG | |
| | ACACTGCCCTCCGAAGTGAATCTGTGCAATATCGACATCT | |
| | TCAACCCGAAGTACGACTGCAAGATCATGACCTCCAAGAC | |
| | CGACGTGTCCAGCAGTGTGATCACCTCTCTGGGCGCCAT | |
| | CGTGTCCTGTTACGGCAAGACCAAGTGCACCGCCAGCAA | |
| | CAAGAACCGGGGCATCATCAAGACCTTCAGCAACGGCTG | |
| | CGACTACGTGTCCAACAAAGGCGTGGACACCGTGTCTGT | |
| | GGGCAACACCCTGTACTACGTGAACAAGCAAGAAGGCAA | |
| | GAGCCTGTACGTGAAGGGCGAGCCCATCATCAACTTCTAC | |
| | GACCCTCTGGTGTTCCCCAGCAGCGAGTTCGATGCCAGC | |
| | ATCTCCCAAGTGAACGAGAAGATCAACCAGAGCCTGGCCT | |
| | TCATCAGAAAGTCCGATGAGCTGCTGCACAACGTGAACGC | |
| | CGGCAAGTCCACCACCAATATCATGATCACGACCATCATC | |
| | ATCGTGATTATCGTGATCCTGCTGGCTCTGATCGCCGTGG | |
| | GCCTGCTGCTGTATTGCAAGGCCAGATCTACCCCAGTGAC | |
| | TCTGTCCAAGGATCAGCTGAGCGGCATCAACAATATCGCC | |
| | TTCTCCAACTGATGAGCTCGCTTTCTTGCTGTCCAATTTCT | |
| | ATTAAAGGTTCCTTTGTTCCCTAAGTCCAACTACTAAACT | |
| | GGGGGATATTATGAAGGGCCTTGAGCATCTGGATTCTGC | |
| | CTAATAAAAAACATTTATTTTCATTGCAAaaaaaaaaaa | |
| | aaaaaaaaaaaaaaaaaaaaaaaaaaaaaaaaaaaaaaa | |
| | aaaaaaaaaaaaaaaaaaaaaaaaaaaaaaa | |
| RSV F | AGGAAATAAGAGAGGATAAGACGACTAAGGAGACATAC | 12 |
| 847B_5UTR_563 | AGAATAAGAAACAGGCAGCCACCATGGAACTGCTGATCC | |
| /3UTR_2 80A | ACAGAAGCAGCGCCATCTTTCTGACCCTGGCCATCAACGC | |
| RSV F 847B | CCTGTACCTGACCAGCAGCCAGAACATCACCGAGGAATTC | |
| DNA | TACCAGAGCACCTGTAGCGCCGTGTCCAGAGGCTACTTTA | |
| Underline = 5′ | GCGCCCTGAGAACCGGCTGGTACACCAGCGTGATCACCA | |
| cap; bold = | TCGAGCTGAGCAACATCAAAGAAACGAAGTGCAACGGCA | |
| 5′UTR | CCGACACCAAAGTGAAGCTGATCAAGCAAGAGCTGGACA | |
| (5UTR_563) and | AGTACAAGAACGCCGTGACCGAACTGCAGCTGCTGATGC | |
| 3′ UTR | AGAATACCCCTGCCTGCAACAACCGGGCCAGAAGAGAAG | |
| (3UTR_2); italics | CCCCTCAGTACATGAACTACACCATCAACACCACCAAGAA | |
| = KOZAK | CCTGAACGTGTCCATCAGCAAGAAGCGGAAGCGGCGGTT | |
| sequence; | CCTGGGCTTTCTGCTTGGAGTGGGAAGCGCCTGCGCCAG | |
| lowercase = | CGGAATCGCCGTGTCTAAAGTGCTGCACCTGGAAGGCGA | |
| polyA tail | AGTGAACAAGATCAAGAATGCCCTGCTGAGCACCAACAAG | |
| | GCCGTGGTGTCTCTGAGCAATGGCGTGTCCGTGCTGACC | |
| | ATCAAGGTGCTGGACCTGAAGAACTACATCAACAACCAGC | |
| | TGCTGCCCATCGTGAACCAGCAGAGCTGCCGGATCAGCA | |
| | ACATCGAGACAGTGATCGAGTTCCAGCAGAAGAACAGCA | |
| | GGCTGCTGGAAATCACCCGCGAGTTCTCTGTGAATGCCG | |
| | GCGTGACAACACCCCTGAGCACCTACATGCTGACCAACA | |
| | GCGAGCTGCTGTCCCTGATCAACGACATGCCCATCACCAA | |
| | CGACCAGAAAAAGCTGATGAGCAGCAACGTGCAGATCGT | |
| | GCGGCAGCAGTCCTACAGCATCATGAGCATTATCAAAGAA | |
| | GAGGTGCTGGCCTACGTGGTGCAGCTGCCTATCTACGGC | |
| | GTGATCGATACCCCTTGCTGGAAGCTGCACACAAGCCCTC | |
| | TGTGCACCACCAATATCAAAGAGGGCTCCAACATCTGCCT | |
| | GACCAGAACCGACAGAGGCTGGTACTGCGATAATGCCGG | |
| | CAGCGTCTCATTCTTCCCACAAGCCGATACCTGCAAGGTG | |
| | CAGAGCAACCGGGTGTTCTGCGACACCATGAACAGCCTG | |
| | ACACTGCCCTCTGAGGTGTCCCTGTGCAACACCGACATCT | |
| | TCAACTCTAAGTACGACTGCAAGATCATGACCAGCAAGAC | |
| | CGATATCAGCTCCTCCGTGATCACAAGCCTGGGCGCCATC | |
| | GTGTCCTGTTACGGCAAGACCAAGTGCACCGCCAGCAAC | |
| | AAGAACCGGGGCATCATCAAGACCTTCAGCAACGGCTGC | |
| | GACTACGTGTCCAACAAAGGCGTGGACACCGTGTCTGTG | |
| | GGCAACACCCTGTACTACGTGAACAAGCTGGAAGGGAAG | |
| | AACCTGTATGTGAAGGGCGAGCCCATCATCAACTACTACG | |
| | ACCCTCTGGTGTTCCCCAGCAGCGAGTTCGATGCCAGCAT | |
| | CAGCCAAGTGAACGAGAAGATCAACCAGAGCCTGGCCTT | |
| | CATCAGGCGGAGCGACGAACTGCTGCACAATGTGAACAC | |
| | CGGCAAGTCCACCACAAACATCATGATCACCGCCATCATC | |
| | ATCGTGATCATTGTGGTGCTGCTGAGCCTGATCGCCATCG | |
| | GCCTGCTGCTGTATTGCAAGGCCAAGAACACCCCAGTGA | |
| | CACTGAGCAAGGATCAGCTGAGCGGCATCAACAATATCGC | |
| | CTTCTCCAAGTGATGAGCTCGCTTTCTTGCTGTCCAATTTC | |
| | TATTAAAGGTTCCTTTGTTCCCTAAGTCCAACTACTAAAC | |
| | TGGGGGATATTATGAAGGGCCTTGAGCATCTGGATTCTG | |
| | CCTAATAAAAAACATTTATTTTCATTGCAAaaaaaaaaa | |
| | aaaaaaaaaaaaaaaaaaaaaaaaaaaaaaaaaaaaaaa | |
| | aaaaaaaaaaaaaaaaaaaaaaaaaaaaaaaa | |
| RSV F | AGGTGTCAGAGTTTAACTTGAAGACTATTTCTAGGGATAA | 13 |
| 847A_5UTR_582 | TATCCCTGCCACCATGGAACTGCCCATCCTGAAAACAAAC | |
| /3UTR_2 80A | GCCATCACCACCATCCTGGCCGCCGTGACACTGTGTTTTG | |
| RSV F 847A | CCAGCAGCCAGAACATCACCGAGGAATTCTACCAGAGCA | |
| DNA | CCTGTAGCGCCGTGTCCAAGGGCTATCTGAGCGCCCTTA | |
| Underline = 5′ | GAACCGGCTGGTACACCAGCGTGATCACCATCGAGCTGA | |
| cap; bold = | GCAACATCAAAGAAAACAAGTGCAACGGCACCGACGCCA | |
| 5′UTR | AAGTGAAGCTGATCAAGCAAGAGCTGGACAAGTACAAGAA | |
| (5UTR_582) and | CGCCGTGACCGAACTGCAGCTGCTGATGCAGTCTACCCC | |
| 3′ UTR | TGCCTGCAATAGCAGAGCCAGACGGGAACTGCCTAGATT | |
| (3UTR_2); italics | CATGAACTACACCCTGAACAACACCAAGAACACCAACGTG | |
| = KOZAK | ACCCTGAGCAAGAAGCGGAAGCGGAGATTCCTGGGCTTT | |
| sequence; | CTGCTCGGAGTGGGAAGCGCCTGCGCCTCTGGAATCGCC | |
| lowercase = | GTGTCTAAAGTGCTGCACCTGGAAGGCGAAGTGAACAAG | |
| polyA tail | ATCAAGAGCGCCCTGCTGAGCACCAACAAGGCCGTGGTG | |
| | TCTCTGAGCAATGGCGTGTCCGTGCTGACCATCAAGGTGC | |
| | TGGACCTGAAGAACTACATCGACAAACAGCTGCTGCCCAT | |
| | CGTCAACAAGCAGAGCTGCAGCATCAGCAACATCGAGAC | |
| | AGTGATCGAGTTCCAGCAGAAGAACAACCGGCTGCTGGA | |
| | AATCACCCGCGAGTTCTCTGTGAATGCCGGCGTGACCACA | |
| | CCTGTGTCCACCTACATGCTGACCAACAGCGAGCTGCTGT | |
| | CCCTGATCAACGACATGCCCATCACCAACGACCAGAAAAA | |
| | GCTGATGAGCAGCAACGTGCAGATCGTGCGGCAGCAGAG | |
| | CTACTCCATCATGAGCATTATCAAAGAAGAGGTGCTGGCC | |
| | TACGTGGTGCAGCTGCCTCTGTATGGCGTGATCGATACCC | |
| | CTTGCTGGAAGCTGCACACAAGCCCTCTGTGCACCACCAA | |
| | CACCAAAGAGGGCTCCAACATCTGCCTGACCAGAACCGA | |
| | CAGAGGCTGGTACTGCGATAATGCCGGCAGCGTCTCATT | |
| | CTTCCCACAAGCCGAGACATGCAAGGTGCAGAGCAACCG | |
| | GGTGTTCTGCGACACCATGAACAGCCTGACACTGCCCTCC | |
| | GAAGTGAATCTGTGCAATATCGACATCTTCAACCCGAAGT | |
| | ACGACTGCAAGATCATGACCTCCAAGACCGACGTGTCCAG | |
| | CAGTGTGATCACCTCTCTGGGCGCCATCGTGTCCTGTTAC | |
| | GGCAAGACCAAGTGCACCGCCAGCAACAAGAACCGGGGC | |
| | ATCATCAAGACCTTCAGCAACGGCTGCGACTACGTGTCCA | |
| | ACAAAGGCGTGGACACCGTGTCTGTGGGCAACACCCTGT | |
| | ACTACGTGAACAAGCAAGAAGGCAAGAGCCTGTACGTGAA | |
| | GGGCGAGCCCATCATCAACTTCTACGACCCTCTGGTGTTC | |
| | CCCAGCAGCGAGTTCGATGCCAGCATCTCCCAAGTGAAC | |
| | GAGAAGATCAACCAGAGCCTGGCCTTCATCAGAAAGTCCG | |
| | ATGAGCTGCTGCACAACGTGAACGCCGGCAAGTCCACCA | |
| | CCAATATCATGATCACGACCATCATCATCGTGATTATCGTG | |
| | ATCCTGCTGGCTCTGATCGCCGTGGGCCTGCTGCTGTATT | |
| | GCAAGGCCAGATCTACCCCAGTGACTCTGTCCAAGGATCA | |
| | GCTGAGCGGCATCAACAATATCGCCTTCTCCAACTGATGA | |
| | GCTCGCTTTCTTGCTGTCCAATTTCTATTAAAGGTTCCTT | |
| | TGTTCCCTAAGTCCAACTACTAAACTGGGGGATATTATGA | |
| | AGGGCCTTGAGCATCTGGATTCTGCCTAATAAAAAACAT | |
| | TTATTTTCATTGCAAaaaaaaaaaaaaaaaaaaaaaaaa | |
| | aaaaaaaaaaaaaaaaaaaaaaaaaaaaaaaaaaaaaaa | |
| | aaaaaaaaaaaaaaaaa | |
| RSV F | AGGTGTCAGAGTTTAACTTGAAGACTATTTCTAGGGATAA | 14 |
| 847B_5UTR_582 | TATCCCTGCCACCATGGAACTGCTGATCCACAGAAGCAGC | |
| /3UTR_2 80A | GCCATCTTTCTGACCCTGGCCATCAACGCCCTGTACCTGA | |
| RSV F 847B | CCAGCAGCCAGAACATCACCGAGGAATTCTACCAGAGCA | |
| DNA | CCTGTAGCGCCGTGTCCAGAGGCTACTTTAGCGCCCTGA | |
| Underline = 5′ | GAACCGGCTGGTACACCAGCGTGATCACCATCGAGCTGA | |
| cap; bold = | GCAACATCAAAGAAACGAAGTGCAACGGCACCGACACCA | |
| 5′UTR | AAGTGAAGCTGATCAAGCAAGAGCTGGACAAGTACAAGAA | |
| (5UTR_582) and | CGCCGTGACCGAACTGCAGCTGCTGATGCAGAATACCCC | |
| 3′ UTR (3UTR_2); | TGCCTGCAACAACCGGGCCAGAAGAGAAGCCCCTCAGTA | |
| italics | CATGAACTACACCATCAACACCACCAAGAACCTGAACGTG | |
| = KOZAK | TCCATCAGCAAGAAGCGGAAGCGGCGGTTCCTGGGCTTT | |
| sequence; | CTGCTTGGAGTGGGAAGCGCCTGCGCCAGCGGAATCGCC | |
| lowercase = | GTGTCTAAAGTGCTGCACCTGGAAGGCGAAGTGAACAAG | |
| polyA tail | ATCAAGAATGCCCTGCTGAGCACCAACAAGGCCGTGGTG | |
| | TCTCTGAGCAATGGCGTGTCCGTGCTGACCATCAAGGTGC | |
| | TGGACCTGAAGAACTACATCAACAACCAGCTGCTGCCCAT | |
| | CGTGAACCAGCAGAGCTGCCGGATCAGCAACATCGAGAC | |
| | AGTGATCGAGTTCCAGCAGAAGAACAGCAGGCTGCTGGA | |
| | AATCACCCGCGAGTTCTCTGTGAATGCCGGCGTGACAACA | |
| | CCCCTGAGCACCTACATGCTGACCAACAGCGAGCTGCTG | |
| | TCCCTGATCAACGACATGCCCATCACCAACGACCAGAAAA | |
| | AGCTGATGAGCAGCAACGTGCAGATCGTGCGGCAGCAGT | |
| | CCTACAGCATCATGAGCATTATCAAAGAAGAGGTGCTGGC | |
| | CTACGTGGTGCAGCTGCCTATCTACGGCGTGATCGATACC | |
| | CCTTGCTGGAAGCTGCACACAAGCCCTCTGTGCACCACCA | |
| | ATATCAAAGAGGGCTCCAACATCTGCCTGACCAGAACCGA | |
| | CAGAGGCTGGTACTGCGATAATGCCGGCAGCGTCTCATT | |
| | CTTCCCACAAGCCGATACCTGCAAGGTGCAGAGCAACCG | |
| | GGTGTTCTGCGACACCATGAACAGCCTGACACTGCCCTCT | |
| | GAGGTGTCCCTGTGCAACACCGACATCTTCAACTCTAAGT | |
| | ACGACTGCAAGATCATGACCAGCAAGACCGATATCAGCTC | |
| | CTCCGTGATCACAAGCCTGGGCGCCATCGTGTCCTGTTAC | |
| | GGCAAGACCAAGTGCACCGCCAGCAACAAGAACCGGGGC | |
| | ATCATCAAGACCTTCAGCAACGGCTGCGACTACGTGTCCA | |
| | ACAAAGGCGTGGACACCGTGTCTGTGGGCAACACCCTGT | |
| | ACTACGTGAACAAGCTGGAAGGGAAGAACCTGTATGTGAA | |
| | GGGCGAGCCCATCATCAACTACTACGACCCTCTGGTGTTC | |
| | CCCAGCAGCGAGTTCGATGCCAGCATCAGCCAAGTGAAC | |
| | GAGAAGATCAACCAGAGCCTGGCCTTCATCAGGCGGAGC | |
| | GACGAACTGCTGCACAATGTGAACACCGGCAAGTCCACC | |
| | ACAAACATCATGATCACCGCCATCATCATCGTGATCATTGT | |
| | GGTGCTGCTGAGCCTGATCGCCATCGGCCTGCTGCTGTA | |
| | TTGCAAGGCCAAGAACACCCCAGTGACACTGAGCAAGGA | |
| | TCAGCTGAGCGGCATCAACAATATCGCCTTCTCCAAGTGA | |
| | TGAGCTCGCTTTCTTGCTGTCCAATTTCTATTAAAGGTTCC | |
| | TTTGTTCCCTAAGTCCAACTACTAAACTGGGGGATATTAT | |
| | GAAGGGCCTTGAGCATCTGGATTCTGCCTAATAAAAAAC | |
| | ATTTATTTTCATTGCAAaaaaaaaaaaaaaaaaaaaaaa | |
| | aaaaaaaaaaaaaaaaaaaaaaaaaaaaaaaaaaaaaaa | |
| | aaaaaaaaaaaaaaaaaaa | |

TABLE 3
RSV F RNA

| | | SEQ |
|---|---|---|
| ID | | ID NO: |
| RSV F 847A | | 15 |
| UGA stop codon; (Amino acid SEQ ID NO: 4) | | |
| RSV F 847B | | 16 |
| UGA stop codon; (Amino acid SEQ ID NO: 6) | | |
| RSV F 851A | | 633 |
| UGA stop codon; (Amino acid SEQ ID NO: 621) | | |
| RSV F 851B | | 634 |
| UGA stop codon; (Amino acid SEQ ID NO: 622) | | |
| RSV F 852A | | 635 |
| UGA stop codon; (Amino acid SEQ ID NO: 623) | | |
| RSV F 852B | | 636 |
| UGA stop codon; (Amino acid SEQ ID NO: 624) | | |
| RSV F 847A Ecto-Foldon | | 637 |
| UGA stop codon; (Amino acid SEQ ID NO: 625) | | |
| RSV F 847B Ecto-Foldon | | 638 |
| UGA stop codon; (Amino acid SEQ ID NO: 626) | | |
| RSV A WT F | | 780 |
| UGA stop codon; (Amino acid SEQ ID NO: 3) | | |
| RSV B WT F | | 781 |
| UGA stop codon; (Amino acid SEQ ID NO: 5) | | |
| | | SEQ |
| ID | Sequence | ID NO: |
| RSV F | AGGAAAUAAGAGAGGAUAAGACGACUAAGGAGACAUACAG | 17 |
| 847A_5UTR | AAUAAGAAACAGGCAGCCACCAUGGAACUGCCCAUCCUGAA | |
| 563/3UTR_2 | AACAAACGCCAUCACCACCAUCCUGGCCGCCGUGACACUGU | |
| 80A_ | GUUUUGCCAGCAGCCAGAACAUCACCGAGGAAUUCUACCAG | |
| modRNA | AGCACCUGUAGCGCCGUGUCCAAGGGCUAUCUGAGCGCCC | |
| Underline = 5′ | UUAGAACCGGCUGGUACACCAGCGUGAUCACCAUCGAGCUG | |
| cap; bold = | AGCAACAUCAAAGAAAACAAGUGCAACGGCACCGACGCCAAA | |
| 5′UTR | GUGAAGCUGAUCAAGCAAGAGCUGGACAAGUACAAGAACGC | |
| (5UTR_563) | CGUGACCGAACUGCAGCUGCUGAUGCAGUCUACCCCUGCCU | |
| and 3′ UTR | GCAAUAGCAGAGCCAGACGGGAACUGCCUAGAUUCAUGAAC | |
| (3UTR_2); | UACACCCUGAACAACACCAAGAACACCAACGUGACCCUGAGC | |
| italics = | AAGAAGCGGAAGCGGAGAUUCCUGGGCUUUCUGCUCGGAG | |
| KOZAK | UGGGAAGCGCCUGCGCCUCUGGAAUCGCCGUGUCUAAAGU | |
| sequence; | GCUGCACCUGGAAGGCGAAGUGAACAAGAUCAAGAGCGCCC | |
| lowercase = | UGCUGAGCACCAACAAGGCCGUGGUGUCUCUGAGCAAUGG | |
| polyA tail | CGUGUCCGUGCUGACCAUCAAGGUGCUGGACCUGAAGAACU | |
| | ACAUCGACAAACAGCUGCUGCCCAUCGUCAACAAGCAGAGC | |
| | UGCAGCAUCAGCAACAUCGAGACAGUGAUCGAGUUCCAGCA | |
| | GAAGAACAACCGGCUGCUGGAAAUCACCCGCGAGUUCUCUG | |
| | UGAAUGCCGGCGUGACCACACCUGUGUCCACCUACAUGCUG | |
| | ACCAACAGCGAGCUGCUGUCCCUGAUCAACGACAUGCCCAU | |
| | CACCAACGACCAGAAAAAGCUGAUGAGCAGCAACGUGCAGA | |
| | UCGUGCGGCAGCAGAGCUACUCCAUCAUGAGCAUUAUCAAA | |
| | GAAGAGGUGCUGGCCUACGUGGUGCAGCUGCCUCUGUAUG | |
| | GCGUGAUCGAUACCCCUUGCUGGAAGCUGCACACAAGCCCU | |
| | CUGUGCACCACCAACACCAAAGAGGGCUCCAACAUCUGCCU | |
| | GACCAGAACCGACAGAGGCUGGUACUGCGAUAAUGCCGGCA | |
| | GCGUCUCAUUCUUCCCACAAGCCGAGACAUGCAAGGUGCAG | |
| | AGCAACCGGGUGUUCUGCGACACCAUGAACAGCCUGACACU | |
| | GCCCUCCGAAGUGAAUCUGUGCAAUAUCGACAUCUUCAACC | |
| | CGAAGUACGACUGCAAGAUCAUGACCUCCAAGACCGACGUG | |
| | UCCAGCAGUGUGAUCACCUCUCUGGGCGCCAUCGUGUCCU | |
| | GUUACGGCAAGACCAAGUGCACCGCCAGCAACAAGAACCGG | |
| | GGCAUCAUCAAGACCUUCAGCAACGGCUGCGACUACGUGUC | |
| | CAACAAAGGCGUGGACACCGUGUCUGUGGGCAACACCCUGU | |
| | ACUACGUGAACAAGCAAGAAGGCAAGAGCCUGUACGUGAAG | |
| | GGCGAGCCCAUCAUCAACUUCUACGACCCUCUGGUGUUCCC | |
| | CAGCAGCGAGUUCGAUGCCAGCAUCUCCCAAGUGAACGAGA | |
| | AGAUCAACCAGAGCCUGGCCUUCAUCAGAAAGUCCGAUGAG | |
| | CUGCUGCACAACGUGAACGCCGGCAAGUCCACCACCAAUAU | |
| | CAUGAUCACGACCAUCAUCAUCGUGAUUAUCGUGAUCCUGC | |
| | UGGCUCUGAUCGCCGUGGGCCUGCUGCUGUAUUGCAAGGC | |
| | CAGAUCUACCCCAGUGACUCUGUCCAAGGAUCAGCUGAGCG | |
| | GCAUCAACAAUAUCGCCUUCUCCAACUGAUGAGCUCGCUUU | |
| | CUUGCUGUCCAAUUUCUAUUAAAGGUUCCUUUGUUCCCUA | |
| | AGUCCAACUACUAAACUGGGGGAUAUUAUGAAGGGCCUUG | |
| | AGCAUCUGGAUUCUGCCUAAUAAAAAACAUUUAUUUUCAUU | |
| | GCAAaaaaaaaaaaaaaaaaaaaaaaaaaaaaaaaaaaaaa | |
| | aaaaaaaaaaaaaaaaaaaaaaaaaaaaaaaaaaaaaaa | |
| | aaaa | |
| RSV F | AGGAAAUAAGAGAGGAUAAGACGACUAAGGAGACAUACAG | 18 |
| 847B_5UTR | AAUAAGAAACAGGCAGCCACCAUGGAACUGCUGAUCCACAG | |
| 563/3UTR_2 | AAGCAGCGCCAUCUUUCUGACCCUGGCCAUCAACGCCCUGU | |
| 80A_modRN | ACCUGACCAGCAGCCAGAACAUCACCGAGGAAUUCUACCAG | |
| A | AGCACCUGUAGCGCCGUGUCCAGAGGCUACUUUAGCGCCC | |
| Underline = 5′ | UGAGAACCGGCUGGUACACCAGCGUGAUCACCAUCGAGCUG | |
| cap; bold = 5′ | AGCAACAUCAAAGAAACGAAGUGCAACGGCACCGACACCAAA | |
| UTR | GUGAAGCUGAUCAAGCAAGAGCUGGACAAGUACAAGAACGC | |
| (5UTR_563) | CGUGACCGAACUGCAGCUGCUGAUGCAGAAUACCCCUGCCU | |
| and 3′ UTR | GCAACAACCGGGCCAGAAGAGAAGCCCCUCAGUACAUGAAC | |
| (3UTR_2); | UACACCAUCAACACCACCAAGAACCUGAACGUGUCCAUCAG | |
| italics = | CAAGAAGCGGAAGCGGCGGUUCCUGGGCUUUCUGCUUGGA | |
| KOZAK | GUGGGAAGCGCCUGCGCCAGCGGAAUCGCCGUGUCUAAAG | |
| sequence; | UGCUGCACCUGGAAGGCGAAGUGAACAAGAUCAAGAAUGCC | |
| lowercase = | CUGCUGAGCACCAACAAGGCCGUGGUGUCUCUGAGCAAUG | |
| polyA tail | GCGUGUCCGUGCUGACCAUCAAGGUGCUGGACCUGAAGAA | |
| | CUACAUCAACAACCAGCUGCUGCCCAUCGUGAACCAGCAGA | |
| | GCUGCCGGAUCAGCAACAUCGAGACAGUGAUCGAGUUCCAG | |
| | CAGAAGAACAGCAGGCUGCUGGAAAUCACCCGCGAGUUCUC | |
| | UGUGAAUGCCGGCGUGACAACACCCCUGAGCACCUACAUGC | |
| | UGACCAACAGCGAGCUGCUGUCCCUGAUCAACGACAUGCCC | |
| | AUCACCAACGACCAGAAAAAGCUGAUGAGCAGCAACGUGCA | |
| | GAUCGUGCGGCAGCAGUCCUACAGCAUCAUGAGCAUUAUCA | |
| | AAGAAGAGGUGCUGGCCUACGUGGUGCAGCUGCCUAUCUA | |
| | CGGCGUGAUCGAUACCCCUUGCUGGAAGCUGCACACAAGCC | |
| | CUCUGUGCACCACCAAUAUCAAAGAGGGCUCCAACAUCUGC | |
| | CUGACCAGAACCGACAGAGGCUGGUACUGCGAUAAUGCCGG | |
| | CAGCGUCUCAUUCUUCCCACAAGCCGAUACCUGCAAGGUGC | |
| | AGAGCAACCGGGUGUUCUGCGACACCAUGAACAGCCUGACA | |
| | CUGCCCUCUGAGGUGUCCCUGUGCAACACCGACAUCUUCAA | |
| | CUCUAAGUACGACUGCAAGAUCAUGACCAGCAAGACCGAUA | |
| | UCAGCUCCUCCGUGAUCACAAGCCUGGGCGCCAUCGUGUC | |
| | CUGUUACGGCAAGACCAAGUGCACCGCCAGCAACAAGAACC | |
| | GGGGCAUCAUCAAGACCUUCAGCAACGGCUGCGACUACGUG | |
| | UCCAACAAAGGCGUGGACACCGUGUCUGUGGGCAACACCCU | |
| | GUACUACGUGAACAAGCUGGAAGGGAAGAACCUGUAUGUGA | |
| | AGGGCGAGCCCAUCAUCAACUACUACGACCCUCUGGUGUUC | |
| | CCCAGCAGCGAGUUCGAUGCCAGCAUCAGCCAAGUGAACGA | |
| | GAAGAUCAACCAGAGCCUGGCCUUCAUCAGGCGGAGCGACG | |
| | AACUGCUGCACAAUGUGAACACCGGCAAGUCCACCACAAAC | |
| | AUCAUGAUCACCGCCAUCAUCAUCGUGAUCAUUGUGGUGCU | |
| | GCUGAGCCUGAUCGCCAUCGGCCUGCUGCUGUAUUGCAAG | |
| | GCCAAGAACACCCCAGUGACACUGAGCAAGGAUCAGCUGAG | |
| | CGGCAUCAACAAUAUCGCCUUCUCCAAGUGAUGAGCUCGCU | |
| | UUCUUGCUGUCCAAUUUCUAUUAAAGGUUCCUUUGUUCCC | |
| | UAAGUCCAACUACUAAACUGGGGGAUAUUAUGAAGGGCCU | |
| | UGAGCAUCUGGAUUCUGCCUAAUAAAAAACAUUUAUUUUC | |
| | AUUGCAAaaaaaaaaaaaaaaaaaaaaaaaaaaaaaaaaa | |
| | aaaaaaaaaaaaaaaaaaaaaaaaaaaaaaaaaaaaaaaa | |
| | aaaaaaa | |
| RSV F | AGGUGUCAGAGUUUAACUUGAAGACUAUUUCUAGGGAUAA | 19 |
| 847A_5UTR_ | UAUCCCUGCCACCAUGGAACUGCCCAUCCUGAAAACAAACG | |
| 582/3UTR_2 | CCAUCACCACCAUCCUGGCCGCCGUGACACUGUGUUUUGCC | |
| 80A_modRN | AGCAGCCAGAACAUCACCGAGGAAUUCUACCAGAGCACCUG | |
| A | UAGCGCCGUGUCCAAGGGCUAUCUGAGCGCCCUUAGAACC | |
| Underline = 5′ | GGCUGGUACACCAGCGUGAUCACCAUCGAGCUGAGCAACAU | |
| cap; bold = | CAAAGAAAACAAGUGCAACGGCACCGACGCCAAAGUGAAGC | |
| 5′UTR | UGAUCAAGCAAGAGCUGGACAAGUACAAGAACGCCGUGACC | |
| (5UTR_582) | GAACUGCAGCUGCUGAUGCAGUCUACCCCUGCCUGCAAUAG | |
| and 3′ UTR | CAGAGCCAGACGGGAACUGCCUAGAUUCAUGAACUACACCC | |
| (3UTR_2); | UGAACAACACCAAGAACACCAACGUGACCCUGAGCAAGAAG | |
| italics = | CGGAAGCGGAGAUUCCUGGGCUUUCUGCUCGGAGUGGGAA | |
| KOZAK | GCGCCUGCGCCUCUGGAAUCGCCGUGUCUAAAGUGCUGCA | |
| sequence; | CCUGGAAGGCGAAGUGAACAAGAUCAAGAGCGCCCUGCUGA | |
| lowercase = | GCACCAACAAGGCCGUGGUGUCUCUGAGCAAUGGCGUGUC | |
| polyA tail | CGUGCUGACCAUCAAGGUGCUGGACCUGAAGAACUACAUCG | |
| | ACAAACAGCUGCUGCCCAUCGUCAACAAGCAGAGCUGCAGC | |
| | AUCAGCAACAUCGAGACAGUGAUCGAGUUCCAGCAGAAGAA | |
| | CAACCGGCUGCUGGAAAUCACCCGCGAGUUCUCUGUGAAUG | |
| | CCGGCGUGACCACACCUGUGUCCACCUACAUGCUGACCAAC | |
| | AGCGAGCUGCUGUCCCUGAUCAACGACAUGCCCAUCACCAA | |
| | CGACCAGAAAAAGCUGAUGAGCAGCAACGUGCAGAUCGUGC | |
| | GGCAGCAGAGCUACUCCAUCAUGAGCAUUAUCAAAGAAGAG | |
| | GUGCUGGCCUACGUGGUGCAGCUGCCUCUGUAUGGCGUGA | |
| | UCGAUACCCCUUGCUGGAAGCUGCACACAAGCCCUCUGUGC | |
| | ACCACCAACACCAAAGAGGGCUCCAACAUCUGCCUGACCAG | |
| | AACCGACAGAGGCUGGUACUGCGAUAAUGCCGGCAGCGUC | |
| | UCAUUCUUCCCACAAGCCGAGACAUGCAAGGUGCAGAGCAA | |
| | CCGGGUGUUCUGCGACACCAUGAACAGCCUGACACUGCCCU | |
| | CCGAAGUGAAUCUGUGCAAUAUCGACAUCUUCAACCCGAAG | |
| | UACGACUGCAAGAUCAUGACCUCCAAGACCGACGUGUCCAG | |
| | CAGUGUGAUCACCUCUCUGGGCGCCAUCGUGUCCUGUUAC | |
| | GGCAAGACCAAGUGCACCGCCAGCAACAAGAACCGGGGCAU | |
| | CAUCAAGACCUUCAGCAACGGCUGCGACUACGUGUCCAACA | |
| | AAGGCGUGGACACCGUGUCUGUGGGCAACACCCUGUACUA | |
| | CGUGAACAAGCAAGAAGGCAAGAGCCUGUACGUGAAGGGCG | |
| | AGCCCAUCAUCAACUUCUACGACCCUCUGGUGUUCCCCAGC | |
| | AGCGAGUUCGAUGCCAGCAUCUCCCAAGUGAACGAGAAGAU | |
| | CAACCAGAGCCUGGCCUUCAUCAGAAAGUCCGAUGAGCUGC | |
| | UGCACAACGUGAACGCCGGCAAGUCCACCACCAAUAUCAUG | |
| | AUCACGACCAUCAUCAUCGUGAUUAUCGUGAUCCUGCUGGC | |
| | UCUGAUCGCCGUGGGCCUGCUGCUGUAUUGCAAGGCCAGA | |
| | UCUACCCCAGUGACUCUGUCCAAGGAUCAGCUGAGCGGCAU | |
| | CAACAAUAUCGCCUUCUCCAACUGAUGAGCUCGCUUUCUUG | |
| | CUGUCCAAUUUCUAUUAAAGGUUCCUUUGUUCCCUAAGUC | |
| | CAACUACUAAACUGGGGGAUAUUAUGAAGGGCCUUGAGCA | |
| | UCUGGAUUCUGCCUAAUAAAAAACAUUUAUUUUCAUUGCA | |
| | Aaaaaaaaaaaaaaaaaaaaaaaaaaaaaaaaaaaaaaaa | |
| | aaaaaaaaaaaaaaaaaaaaaaaaaaaaaaaaaaaaaaaaa | |
| RSV F | AGGUGUCAGAGUUUAACUUGAAGACUAUUUCUAGGGAUAA | 20 |
| 847B_5UTR_ | UAUCCCUGCCACCAUGGAACUGCUGAUCCACAGAAGCAGCG | |
| 582/3UTR_2 | CCAUCUUUCUGACCCUGGCCAUCAACGCCCUGUACCUGACC | |
| 80A_modRN | AGCAGCCAGAACAUCACCGAGGAAUUCUACCAGAGCACCUG | |
| A | UAGCGCCGUGUCCAGAGGCUACUUUAGCGCCCUGAGAACC | |
| Underline = 5′ | GGCUGGUACACCAGCGUGAUCACCAUCGAGCUGAGCAACAU | |
| cap; bold = | CAAAGAAACGAAGUGCAACGGCACCGACACCAAAGUGAAGC | |
| 5′UTR | UGAUCAAGCAAGAGCUGGACAAGUACAAGAACGCCGUGACC | |
| (5UTR_582) | GAACUGCAGCUGCUGAUGCAGAAUACCCCUGCCUGCAACAA | |
| and 3′ UTR | CCGGGCCAGAAGAGAAGCCCCUCAGUACAUGAACUACACCA | |
| (3UTR_2); | UCAACACCACCAAGAACCUGAACGUGUCCAUCAGCAAGAAG | |
| italics = | CGGAAGCGGCGGUUCCUGGGCUUUCUGCUUGGAGUGGGAA | |
| KOZAK | GCGCCUGCGCCAGCGGAAUCGCCGUGUCUAAAGUGCUGCA | |
| sequence; | CCUGGAAGGCGAAGUGAACAAGAUCAAGAAUGCCCUGCUGA | |
| lowercase = | GCACCAACAAGGCCGUGGUGUCUCUGAGCAAUGGCGUGUC | |
| polyA tail | CGUGCUGACCAUCAAGGUGCUGGACCUGAAGAACUACAUCA | |
| | ACAACCAGCUGCUGCCCAUCGUGAACCAGCAGAGCUGCCGG | |
| | AUCAGCAACAUCGAGACAGUGAUCGAGUUCCAGCAGAAGAA | |
| | CAGCAGGCUGCUGGAAAUCACCCGCGAGUUCUCUGUGAAUG | |
| | CCGGCGUGACAACACCCCUGAGCACCUACAUGCUGACCAAC | |
| | AGCGAGCUGCUGUCCCUGAUCAACGACAUGCCCAUCACCAA | |
| | CGACCAGAAAAAGCUGAUGAGCAGCAACGUGCAGAUCGUGC | |
| | GGCAGCAGUCCUACAGCAUCAUGAGCAUUAUCAAAGAAGAG | |
| | GUGCUGGCCUACGUGGUGCAGCUGCCUAUCUACGGCGUGA | |
| | UCGAUACCCCUUGCUGGAAGCUGCACACAAGCCCUCUGUGC | |
| | ACCACCAAUAUCAAAGAGGGCUCCAACAUCUGCCUGACCAG | |
| | AACCGACAGAGGCUGGUACUGCGAUAAUGCCGGCAGCGUCU | |
| | CAUUCUUCCCACAAGCCGAUACCUGCAAGGUGCAGAGCAAC | |
| | CGGGUGUUCUGCGACACCAUGAACAGCCUGACACUGCCCUC | |
| | UGAGGUGUCCCUGUGCAACACCGACAUCUUCAACUCUAAGU | |
| | ACGACUGCAAGAUCAUGACCAGCAAGACCGAUAUCAGCUCC | |
| | UCCGUGAUCACAAGCCUGGGCGCCAUCGUGUCCUGUUACG | |
| | GCAAGACCAAGUGCACCGCCAGCAACAAGAACCGGGGCAUC | |
| | AUCAAGACCUUCAGCAACGGCUGCGACUACGUGUCCAACAA | |
| | AGGCGUGGACACCGUGUCUGUGGGCAACACCCUGUACUAC | |
| | GUGAACAAGCUGGAAGGGAAGAACCUGUAUGUGAAGGGCGA | |
| | GCCCAUCAUCAACUACUACGACCCUCUGGUGUUCCCCAGCA | |
| | GCGAGUUCGAUGCCAGCAUCAGCCAAGUGAACGAGAAGAUC | |
| | AACCAGAGCCUGGCCUUCAUCAGGCGGAGCGACGAACUGCU | |
| | GCACAAUGUGAACACCGGCAAGUCCACCACAAACAUCAUGA | |
| | UCACCGCCAUCAUCAUCGUGAUCAUUGUGGUGCUGCUGAGC | |
| | CUGAUCGCCAUCGGCCUGCUGCUGUAUUGCAAGGCCAAGAA | |
| | CACCCCAGUGACACUGAGCAAGGAUCAGCUGAGCGGCAUCA | |
| | ACAAUAUCGCCUUCUCCAAGUGAUGAGCUCGCUUUCUUGCU | |
| | GUCCAAUUUCUAUUAAAGGUUCCUUUGUUCCCUAAGUCCA | |
| | ACUACUAAACUGGGGGAUAUUAUGAAGGGCCUUGAGCAUC | |
| | UGGAUUCUGCCUAAUAAAAAACAUUUAUUUUCAUUGCAAaa | |
| | aaaaaaaaaaaaaaaaaaaaaaaaaaaaaaaaaaaaaaaa | |
| | aaaaaaaaaaaaaaaaaaaaaaaaaaaaaaaaaaaaaa | |

B. hMPV Sequences

TABLE 4
hMPV F Polypeptides

| ID | SEQ ID NO: |
|---|---|
| Full Length F0 of Native hMPV A2b (GenBank GI: | 639 |
| ACJ53569.1) | |
| Full Length F0 of Native hMPV B2 (GenBank GI: | 640 |
| QDA18370.1) | |
| hMPV F hMPV021 A | 641 |
| hMPV F hMPV029 A | 642 |
| hMPV F hMPV198 A | 643 |
| hMPV F hMPV189 B | 644 |
| hMPV F hMPV194 B | 645 |
| hMPV F hMPV029 A Ecto-foldon | 646 |
| hMPV F hMPV189 B Ecto-foldon | 647 |
| hMPV F hMPV198 B Ecto-foldon | 715 |
| hMPV F hMPV194 B Ecto-foldon | 716 |
| hMPV F hMPV164 B | 721 |

TABLE 5
hMPV F DNA

| | | SEQ |
|---|---|---|
| ID | | ID NO: |
| hMPV A WT F | | 648 |
| TGA stop codon; (Amino acid SEQ ID NO: 639) | | |
| hMPV F hMPV021 A | | 649 |
| TGA stop codon; (Amino acid SEQ ID NO: 641) | | |
| hMPV F hMPV029 A | | 650 |
| TGA stop codon; (Amino acid SEQ ID NO:642) | | |
| hMPV F hMPV198 A | | 651 |
| TGA stop codon; (Amino acid SEQ ID NO: 643) | | |
| hMPV B WT F | | 652 |
| TGA stop codon; (Amino acid SEQ ID NO: 640) | | |
| hMPV F hMPV189 B | | 653 |
| TGA stop codon; (Amino acid SEQ ID NO: 644) | | |
| hMPV F hMPV194 B | | 654 |
| TGA stop codon; (Amino acid SEQ ID NO: 645) | | |
| hMPV F hMPV029 A Ecto-foldon | | 659 |
| TGA stop codon; (Amino acid SEQ ID NO: 646) | | |
| hMPV F hMPV189 B Ecto-foldon | | 660 |
| TGA stop codon; (Amino acid SEQ ID NO: 647) | | |
| hMPV F hMPV198 A Ecto-foldon | | 717 |
| TGA stop codon; (Amino acid SEQ ID NO: 715) | | |
| hMPV F hMPV194 B Ecto-foldon | | 718 |
| TGA stop codon; (Amino acid SEQ ID NO: 716) | | |
| hMPV F hMPV164 B | | 722 |
| TGA stop codon; (Amino acid SEQ ID NO: 721) | | |
| | | SEQ |
| ID | hMPV Sequence | ID NO: |
| hMPV029_5UT | AGGTGTCAGAGTTTAACTTGAAGACTATTTCTAGGGATAA | 655 |
| R_582/3UTR_2 | TATCCCTGCCACCATGAGCTGGAAGGTGGTCATCATCTTCA | |
| 80A | GCCTGCTGATCACCCCTCAGCACGGCCTGAAAGAGAGCTA | |
| hMPV F | CCTGGAAGAAAGCTGCAGCACCATCACCGAGGGCTACCTG | |
| hMPV029 A | AGCGTGCTGAGAACCGGCTGGTACACCAACGTGTTCACCC | |
| DNA | TGGAAGTGGGCGACGTGGAAAACCTGACCTGTGCCGATG | |
| Underline = 5′ | GACCCAGCCTGATCAAGACCGAGCTGGACCTGACAAAGAG | |
| cap; bold = | CGCCCTGCGGGAACTGAAAACCGTGTCTGCAGATCAGCTG | |
| 5′UTR | GCCAGAGAGGAACAGATCGAGAACCCCAGACGGCGGAGA | |
| (5UTR_582) and | TTCGTGCTGGGAGCTATCGCCTGCGGAGTTGCTACAGCTG | |
| 3′ UTR | CTGCTGTGACAGCTGGCGTGGCCATTGCCAAGTGCATCCG | |
| (3UTR_2); | GCTGGAAAGCGAAGTGACCGCCATCAAGAACTGCCTGAAA | |
| italics = KOZAK | AAGACCAACGAGTGCGTGTCTACCCTCGGCTGCGGCGTTA | |
| sequence; | GAGTGCTGGCCACAGCCGTCCGCGAGCTGAAGGATTTCGT | |
| lowercase = | GTCCAAGAACCTGACCAGGGCCATCAACAAGAACAAGTGC | |
| polyA tail | GACATCCCCGACCTGAAGATGGCCGTGTCCTTCAGCCAGT | |
| (Amino acid | TCAACCGGCGGTTCCTGAATGTCGTGCGGCAGTTCTCTGA | |
| SEQ ID NO: | CAACGCCGGCATCACACCAGCCATCAGCAAGGATCTGATG | |
| 642) | ACCGATGCCGAACTGGCTAGAGCCATCTCCAACATGCCTA | |
| | CATCTGCCGGCCAGATCAAGCTGATGCTGGAAAACAGAGC | |
| | CATGGTCCGACGGAAAGGCTTCGGCATCCTGATCGGCGTG | |
| | TACGGCAGCAGCGTGATCTACATGGTGCAGCTGCCTATCT | |
| | TCGGCGTGATCGACACCCCTTGCTGGATCGTGAAAGCCGC | |
| | TCCTAGCTGCAGCGAGAAGAAGGGCAATTACGCCTGCCTG | |
| | CTGAGAGAGGACCAAGGCTGGTACTGTCAGAATGCCGGCA | |
| | GCACCGTGTACTACCCCTGCGAGAAGGACTGCGAGACAAG | |
| | AGGCGACCACGTGTTCTGTGATACCGCCGCTGGAATCAAC | |
| | GTGGCCGAGCAGAGCAAAGAGTGCAACATCAACATCAGCA | |
| | CCACAAACTACCCCTGCAAGGTGTCCTGCGGCAGACACCC | |
| | TATCAGCATGGTGGCTCTGTCTCCACTGGGAGCCCTGGTG | |
| | GCTTGTTATAAGGGCGTGTCCTGTAGCATCGGCAGCAACA | |
| | GAGTGGGCATCATCAAGCAGCTGAACAAGGGCTGCTCCTA | |
| | CATCACCAACCAGGACGCCGATACCGTGACCATCGACAAT | |
| | ACCGTGTATCAGCTGAGCAAGGTGGAAGGCGAACAGCACG | |
| | TGATCAAGGGCAGACCTGTGTCCAGCAGCTTCGACCCCGT | |
| | GAAGTTCCCCCAGGACCAGTTCAATGTGGCCCTGGACCAG | |
| | TGCTTCGAGAACATCGAGAACTCTCAGGCTCTGGTGGACC | |
| | AGTCCAACCGGATTCTGTCTAGCGCCGAGAAGGGAAACAC | |
| | CGGCTTCATCATCGTGATCATCCTGATTGCCGTGCTGGGC | |
| | TCCAGCATGATCCTGGTGTCCATCTTTATCATCATCAAAAA | |
| | GACGAAGAAGCCCACAGGCGCCCCTCCAGAACTGTCTGG | |
| | CGTGACCAACAATGGCTTCATCCCTCACAGCTGATGAGCT | |
| | CGCTTTCTTGCTGTCCAATTTCTATTAAAGGTTCCTTTGTT | |
| | CCCTAAGTCCAACTACTAAACTGGGGGATATTATGAAGGGC | |
| | CTTGAGCATCTGGATTCTGCCTAATAAAAAACATTTATTTT | |
| | CATTGCAAaaaaaaaaaaaaaaaaaaaaaaaaaaaaaaaaaa | |
| | aaaaaaaaaaaaaaaaaaaaaaaaaaaaaaaaaaaaaaaaa | |
| | aaaaa | |
| hMPV198_5UT | AGGTGTCAGAGTTTAACTTGAAGACTATTTCTAGGGATAA | 656 |
| R_582/3UTR_2 | TATCCCTGCCACCATGAGCTGGAAGGTGGTCATCATCTTCA | |
| 80A | GCCTGCTGATCACCCCTCAGCACGGCCTGAAAGAGAGCTA | |
| hMPV F | CCTGGAAGAAAGCTGCAGCACCATCACCGAGGGCTACCTG | |
| hMPV198 A | AGCGTGCTGAGAACCGGCTGGTACACCAACGTGTTCACCC | |
| DNA | TGGAAGTGGGCGACGTGGAAAACCTGACCTGTGCCGATG | |
| Underline = 5′ | GACCCAGCCTGATCAAGACCGAGCTGGACCTGACAAAGAG | |
| cap; bold = | CGCCCTGCGGGAACTGAAAACCTGCTCTGCAGATCAGGGC | |
| 5′UTR | AGCGGCGGCAGCGGCGCTACAGCTGCTGCTGTGACAGCT | |
| (5UTR_582) and | GGCGTGGCCATTGCCAAGACCATCCGGCTGGAAAGCGAA | |
| 3′ UTR | GTGACCGCCATCAAGAACTGCCTGAAAAAGACCAACGAGT | |
| (3UTR_2); | GCGTGTCTACCCTCGGCAATGGCGTTAGAGTGCTGGCCAC | |
| italics = KOZAK | AGCCGTCCGCGAGCTGAAGGATTTCGTGTCCAAGAACCTG | |
| sequence; | ACCAGGGCCATCAACAAGAACAAGTGCGACATCCCCGACC | |
| lowercase = | TGAAGATGGCCGTGTCCTTCAGCCAGTTCAACCGGCGGTT | |
| polyA tail | CCTGAATGTCGTGCGGCAGTTCTCTGACAACGCCGGCATC | |
| (Amino acid | ACACCAGCCATCAGCCTGGATCTGATGACCGATGCCGAAC | |
| SEQ ID NO: | TGGCTAGAGCCGTGTCCAACATGCCTACATCTGCCGGCCA | |
| 643) | GATCAAGCTGATGCTGGAAAACAGATGCATGGTCCGACGG | |
| | AAAGGCTTCGGCATCCTGATCGGCGTGTACGGCAGCAGCG | |
| | TGATCTACATGGTGCAGCTGCCTATCTTCGGCGTGATCGA | |
| | CACCCCTTGCTGGATCGTGAAAGCCGCTCCTAGCTGCAGC | |
| | GAGAAGAAGGGCAATTACGCCTGCCTGCTGAGAGAGGACC | |
| | AAGGCTGGTACTGTCAGAATGCCGGCAGCACCGTGTACTA | |
| | CCCCAACGAGAAGGACTGCGAGACAAGAGGCGACCACGT | |
| | GTTCTGTGATACCGCCGCTGGAATCAACGTGGCCGAGCAG | |
| | AGCAAAGAGTGCAACATCAACATCAGCACCACAAACTACCC | |
| | CTGCAAGGTGTCCACCGGCAGACACCCTATCAGCATGGTG | |
| | GCTCTGTCTCCACTGGGAGCCCTGGTGGCTTGTTATAAGG | |
| | GCGTGTCCTGTAGCATCGGCAGCAACAGAGTGGGCATCAT | |
| | CAAGCAGCTGAACAAGGGCTGCTCCTACATCACCAACCAG | |
| | GACGCCGATACCGTGACCATCGACAATACCGTGTATCAGC | |
| | TGAGCAAGGTGGAAGGCGAACAGCACGTGATCAAGGGCA | |
| | GACCTGTGTCCAGCAGCTTCGACCCCGTGAAGTTCCCCGA | |
| | GTGCCAGTTCAATTGCGCCCTGGACCAGGTGTTCGAGAAC | |
| | ATCGAGAACTCTCAGGCTCTGGTGGACCAGTCCAACCGGA | |
| | TTCTGTCTAGCGCCGAGAAGGGAAACACCGGCTTCATCAT | |
| | CGTGATCATCCTGATTGCCGTGCTGGGCTCCAGCATGATC | |
| | CTGGTGTCCATCTTTATCATCATCAAAAAGACGAAGAAGCC | |
| | CACAGGCGCCCCTCCAGAACTGTCTGGCGTGACCAACAAT | |
| | GGCTTCATCCCTCACAGCTGATGAGCTCGCTTTCTTGCTGT | |
| | CCAATTTCTATTAAAGGTTCCTTTGTTCCCTAAGTCCAACT | |
| | ACTAAACTGGGGGATATTATGAAGGGCCTTGAGCATCTG | |
| | GATTCTGCCTAATAAAAAACATTTATTTTCATTGCAAaaaaa | |
| | aaaaaaaaaaaaaaaaaaaaaaaaaaaaaaaaaaaaaaaaa | |
| | aaaaaaaaaaaaaaaaaaaaaaaaaaaaaaaaaa | |
| hMPV189_5UT | AGGTGTCAGAGTTTAACTTGAAGACTATTTCTAGGGATAA | 657 |
| R_582/3UTR_2 | TATCCCTGCCACCATGAGCTGGAAAGTCATGATCATCATCA | |
| 80A | GCCTGCTGATCACCCCTCAGCACGGCCTGAAAGAGAGCTA | |
| hMPV F | CCTGGAAGAAAGCTGCAGCACCATCACCGAGGGCTACCTG | |
| hMPV189 B | AGCGTGCTGAGAACCGGCTGGTACACCAACGTGTTCACCC | |
| DNA | TGGAAGTGGGCGACGTGGAAAACCTGACCTGCACAGATGG | |
| Underline = 5′ | CCCCAGCCTGATCAAGACCGAGCTGGATCTGACAAAGAGC | |
| cap; bold = | GCCCTGCGCGAGCTGAAAACCGTGTCTGCAGATCAGCTGG | |
| 5′UTR | CCAGAGAGGAACAGATCGAGAACCCCAGACGGCGGAGAT | |
| (5UTR_582) and | TCGTGCTGGGAGCTATCGCCTGCGGAGTTGCTACAGCTGC | |
| 3′ UTR | TGCTGTGACAGCCGGAATCGCCATTGCCAAGTGCATCCGG | |
| (3UTR_2); | CTGGAAAGCGAAGTGAACGCCATCAAGGGCTGCCTGAAAA | |
| italics = KOZAK | CCACCAACGAGTGCGTGTCTACCCTCGGCTGCGGTGTTAG | |
| sequence; | AGTGCTGGCCACAGCCGTGCGGGAACTGAAAGAATTCGTG | |
| lowercase = | TCCAAGAACCTGACCAGCGCCATCAACAAGAACAAGTGCG | |
| polyA tail | ACATTCCCGACCTGAAGATGGCCGTGTCCTTCAGCCAGTT | |
| (Amino acid | CAACCGGCGGTTCCTGAATGTCGTGCGGCAGTTCTCTGAC | |
| SEQ ID NO: | AACGCCGGCATCACACCAGCCATTAGCAAGGACCTGATGA | |
| 644) | ACGACGCCGAACTGGCTAGAGCCATCTCTTACATGCCTAC | |
| | CTCTGCCGGCCAGATCAAGCTGATGCTGGAAAACAGAGCC | |
| | ATGGTCCGACGGAAAGGCTTCGGCATCCTGATCGGCGTGT | |
| | ACGGCAGCAGCGTGATCTACATGGTGCAGCTGCCTATCTT | |
| | CGGCGTGATCAACACCCCTTGCTGGATCATCAAGGCCGCT | |
| | CCTAGCTGCAGCGAGAAGGACGGCAATTACGCCTGCCTGC | |
| | TGAGAGAGGACCAAGGCTGGTACTGCAAGAATGCCGGCA | |
| | GCACCGTGTACTACCCCTGCGAGAAGGATTGCGAGACACG | |
| | GGGCGATCACGTGTTCTGTGATACAGCCGCCGGAATCAAC | |
| | GTGGCCGAGCAGAGCAGAGAGTGCAACATCAACATCAGCA | |
| | CCACAAACTACCCCTGCAAGGTGTCCTGCGGCAGACACCC | |
| | TATCAGCATGGTGGCTCTGTCTCCACTGGGAGCCCTGGTG | |
| | GCTTGTTATAAGGGCGTGTCCTGTAGCATCGGCAGCAATC | |
| | AAGTGGGCATCATCAAGCAGCTGCCCAAGGGCTGCTCCTA | |
| | CATCACCAATCAGGACGCCGACACCGTGACCATCGACAAT | |
| | ACCGTGTATCAGCTGAGCAAGGTGGAAGGCGAACAGCACG | |
| | TGATCAAGGGCAGACCTGTGTCCAACAGCTTCGACCCCAT | |
| | CAGATTCCCCCAGGACCAGTTCAATGTGGCCCTGGACCAG | |
| | TGCTTCGAGAGCATCGAGAATAGCCAGGCTCTGGTGGACC | |
| | AGTCCAACAAGATCCTGAACTCCGCCGAGAAGGGCAACAC | |
| | CGGCTTCATCATCGTGATCATCCTGATTGCCGTGCTGGGC | |
| | CTGACCATGATCAGCGTGTCCATCATCATTATCATCAAGAA | |
| | AACGCGGAAGCCCGCCGGCGCCCCTCCAGAACTTAATGG | |
| | CGTGACCAACGGCGGCTTCATTCCCCACTCTTGATGAGCT | |
| | CGCTTTCTTGCTGTCCAATTTCTATTAAAGGTTCCTTTGTT | |
| | CCCTAAGTCCAACTACTAAACTGGGGGATATTATGAAGGGC | |
| | CTTGAGCATCTGGATTCTGCCTAATAAAAAACATTTATTTT | |
| | CATTGCAAaaaaaaaaaaaaaaaaaaaaaaaaaaaaaaaaa | |
| | aaaaaaaaaaaaaaaaaaaaaaaaaaaaaaaaaaaaaaaa | |
| | aaaaaaa | |
| hMPV194_5UT | AGGTGTCAGAGTTTAACTTGAAGACTATTTCTAGGGATAA | 658 |
| R_582/3UTR_2 | TATCCCTGCCACCATGAGCTGGAAAGTCATGATCATCATCA | |
| 80A | GCCTGCTGATCACCCCTCAGCACGGCCTGAAAGAGAGCTA | |
| hMPV F | CCTGGAAGAAAGCTGCAGCACCATCACCGAGGGCTACCTG | |
| hMPV194 B | AGCGTGCTGAGAACCGGCTGGTACACCAACGTGTTCACCC | |
| DNA | TGGAAGTGGGCGACGTGGAAAACCTGACCTGCACAGATGG | |
| Underline = 5′ | CCCCAGCCTGATCAAGACCGAGCTGGATCTGACAAAGAGC | |
| cap; bold = | GCCCTGCGCGAGCTGAAAACCTGCTCTGCAGATCAGGGCA | |
| 5′UTR | GCGGCGGCAGCGGCGCTACAGCTGCTGCTGTGACAGCCG | |
| (5UTR_582) and | GAATCGCCATTGCCAAGACCATCCGGCTGGAAAGCGAAGT | |
| 3′ UTR | GAACGCCATCAAGGGCTGCCTGAAAACCACCAACGAGTGC | |
| (3UTR_2); | GTGTCTACCCTCGGCAACGGTGTTAGAGTGCTGGCCACAG | |
| italics = KOZAK | CCGTGCGGGAACTGAAAGAATTCGTGTCCAAGAACCTGAC | |
| sequence; | CAGCGCCATCAACAAGAACAAGTGCGACATTCCCGACCTG | |
| lowercase = | AAGATGGCCGTGTCCTTCAGCCAGTTCAACCGGCGGTTCC | |
| polyA tail | TGAATGTCGTGCGGCAGTTCTCTGACAACGCCGGCATCAC | |
| (Amino acid | ACCAGCCATTAGCCTGGACCTGATGAACGACGCCGAACTG | |
| SEQ ID NO: | GCTAGAGCCGTGTCTTACATGCCTACCTCTGCCGGCCAGA | |
| 645) | TCAAGCTGATGCTGGAAAACAGATGCATGGTCCGACGGAA | |
| | AGGCTTCGGCATCCTGATCGGCGTGTACGGCAGCAGCGT | |
| | GATCTACATGGTGCAGCTGCCTATCTTCGGCGTGATCAACA | |
| | CCCCTTGCTGGATCATCAAGGCCGCTCCTAGCTGCAGCGA | |
| | GAAGGACGGCAATTACGCCTGCCTGCTGAGAGAGGACCAA | |
| | GGCTGGTACTGCAAGAATGCCGGCAGCACCGTGTACTACC | |
| | CCAACGAGAAGGATTGCGAGACACGGGGCGATCACGTGTT | |
| | CTGTGATACAGCCGCCGGAATCAACGTGGCCGAGCAGAG | |
| | CAGAGAGTGCAACATCAACATCAGCACCACAAACTACCCCT | |
| | GCAAGGTGTCCACCGGCAGACACCCTATCAGCATGGTGGC | |
| | TCTGTCTCCACTGGGAGCCCTGGTGGCTTGTTATAAGGGC | |
| | GTGTCCTGTAGCATCGGCAGCAATCAAGTGGGCATCATCA | |
| | AGCAGCTGCCCAAGGGCTGCTCCTACATCACCAATCAGGA | |
| | CGCCGACACCGTGACCATCGACAATACCGTGTATCAGCTG | |
| | AGCAAGGTGGAAGGCGAACAGCACGTGATCAAGGGCAGA | |
| | CCTGTGTCCAACAGCTTCGACCCCATCAGATTCCCCGAGT | |
| | GCCAGTTCAATTGCGCCCTGGACCAGGTGTTCGAGAGCAT | |
| | CGAGAATAGCCAGGCTCTGGTGGACCAGTCCAACAAGATC | |
| | CTGAACTCCGCCGAGAAGGGCAACACCGGCTTCATCATCG | |
| | TGATCATCCTGATTGCCGTGCTGGGCCTGACCATGATCAG | |
| | CGTGTCCATCATCATTATCATCAAGAAAACGCGGAAGCCCG | |
| | CCGGCGCCCCTCCAGAACTTAATGGCGTGACCAACGGCG | |
| | GCTTCATTCCCCACTCTTGATGAGCTCGCTTTCTTGCTGTC | |
| | CAATTTCTATTAAAGGTTCCTTTGTTCCCTAAGTCCAACTA | |
| | CTAAACTGGGGGATATTATGAAGGGCCTTGAGCATCTGG | |
| | ATTCTGCCTAATAAAAAACATTTATTTTCATTGCAAaaaa | |
| | aaaaaaaaaaaaaaaaaaaaaaaaaaaaaaaaaaaaaaaa | |
| | aaaaaaaaaaaaaaaaaaaaaaaaaaaaaaaaaaaa | |

TABLE 6
hMPV F RNA

| | SEQ ID |
|---|---|
| ID | NO: |
| hMPV F hMPV021 A | 661 |
| UGA stop codon; (Amino acid SEQ ID NO: 641) | |
| hMPV F hMPV029 A | 662 |
| UGA stop codon; (Amino acid SEQ ID NO: 642) | |
| hMPV F hMPV198 A | 663 |
| UGA stop codon; (Amino acid SEQ ID NO: 643) | |
| hMPV F hMPV189 B | 664 |
| UGA stop codon; (Amino acid SEQ ID NO: 644) | |
| hMPV F hMPV194 B | 665 |
| UGA stop codon; (Amino acid SEQ ID NO: 645) | |
| hMPV F hMPV029 A Ecto-foldon | 670 |
| UGA stop codon; (Amino acid SEQ ID NO: 646) | |
| hMPV F hMPV189 B Ecto-foldon | 671 |
| UGA stop codon; (Amino acid SEQ ID NO: 647) | |
| hMPV F hMPV198 A Ecto-foldon | 719 |
| UGA stop codon; (Amino acid SEQ ID NO: 715) | |
| hMPV F hMPV194 B Ecto-foldon | 720 |
| UGA stop codon; (Amino acid SEQ ID NO: 716) | |
| hMPV F hMPV164 B | 723 |
| UGA stop codon; (Amino acid SEQ ID NO: 721) | |
| hMPV A WT F | 764 |
| UGA stop codon; (Amino acid SEQ ID NO: 639) | |
| hMPV B WT F | 765 |
| UGA stop codon; (Amino acid SEQ ID NO: 640) | |

| | | SEQ ID |
|---|---|---|
| ID | Sequence | NO: |
| hMPV029_5UTR_582/ | AGGUGUCAGAGUUUAACUUGAAGACUAUUUCUAGGGAUA | 666 |
| 3UTR_2 80A_modRNA | AUAUCCCUGCCACCAUGAGCUGGAAGGUGGUCAUCAUCU | |
| Underline = 5′ cap; | UCAGCCUGCUGAUCACCCCUCAGCACGGCCUGAAAGAGA | |
| bold = 5′UTR | GCUACCUGGAAGAAAGCUGCAGCACCAUCACCGAGGGCU | |
| (5UTR_582) and | ACCUGAGCGUGCUGAGAACCGGCUGGUACACCAACGUGU | |
| 3′ UTR (3UTR_2); | UCACCCUGGAAGUGGGCGACGUGGAAAACCUGACCUGUG | |
| italics = KOZAK | CCGAUGGACCCAGCCUGAUCAAGACCGAGCUGGACCUGA | |
| sequence; | CAAAGAGCGCCCUGCGGGAACUGAAAACCGUGUCUGCAG | |
| lowercase = | AUCAGCUGGCCAGAGAGGAACAGAUCGAGAACCCCAGAC | |
| polyA tail | GGCGGAGAUUCGUGCUGGGAGCUAUCGCCUGCGGAGUUG | |
| (Amino acid | CUACAGCUGCUGCUGUGACAGCUGGCGUGGCCAUUGCCA | |
| SEQ ID NO: 642) | AGUGCAUCCGGCUGGAAAGCGAAGUGACCGCCAUCAAGA | |
| | ACUGCCUGAAAAAGACCAACGAGUGCGUGUCUACCCUCG | |
| | GCUGCGGCGUUAGAGUGCUGGCCACAGCCGUCCGCGAGC | |
| | UGAAGGAUUUCGUGUCCAAGAACCUGACCAGGGCCAUCA | |
| | ACAAGAACAAGUGCGACAUCCCCGACCUGAAGAUGGCCG | |
| | UGUCCUUCAGCCAGUUCAACCGGCGGUUCCUGAAUGUCG | |
| | UGCGGCAGUUCUCUGACAACGCCGGCAUCACACCAGCCA | |
| | UCAGCAAGGAUCUGAUGACCGAUGCCGAACUGGCUAGAG | |
| | CCAUCUCCAACAUGCCUACAUCUGCCGGCCAGAUCAAGC | |
| | UGAUGCUGGAAAACAGAGCCAUGGUCCGACGGAAAGGCU | |
| | UCGGCAUCCUGAUCGGCGUGUACGGCAGCAGCGUGAUCU | |
| | ACAUGGUGCAGCUGCCUAUCUUCGGCGUGAUCGACACCC | |
| | CUUGCUGGAUCGUGAAAGCCGCUCCUAGCUGCAGCGAGA | |
| | AGAAGGGCAAUUACGCCUGCCUGCUGAGAGAGGACCAAG | |
| | GCUGGUACUGUCAGAAUGCCGGCAGCACCGUGUACUACC | |
| | CCUGCGAGAAGGACUGCGAGACAAGAGGCGACCACGUGU | |
| | UCUGUGAUACCGCCGCUGGAAUCAACGUGGCCGAGCAGA | |
| | GCAAAGAGUGCAACAUCAACAUCAGCACCACAAACUACC | |
| | CCUGCAAGGUGUCCUGCGGCAGACACCCUAUCAGCAUGG | |
| | UGGCUCUGUCUCCACUGGGAGCCCUGGUGGCUUGUUAUA | |
| | AGGGCGUGUCCUGUAGCAUCGGCAGCAACAGAGUGGGCA | |
| | UCAUCAAGCAGCUGAACAAGGGCUGCUCCUACAUCACCA | |
| | ACCAGGACGCCGAUACCGUGACCAUCGACAAUACCGUGU | |
| | AUCAGCUGAGCAAGGUGGAAGGCGAACAGCACGUGAUCA | |
| | AGGGCAGACCUGUGUCCAGCAGCUUCGACCCCGUGAAGU | |
| | UCCCCCAGGACCAGUUCAAUGUGGCCCUGGACCAGUGCU | |
| | UCGAGAACAUCGAGAACUCUCAGGCUCUGGUGGACCAGU | |
| | CCAACCGGAUUCUGUCUAGCGCCGAGAAGGGAAACACCG | |
| | GCUUCAUCAUCGUGAUCAUCCUGAUUGCCGUGCUGGGCU | |
| | CCAGCAUGAUCCUGGUGUCCAUCUUUAUCAUCAUCAAAA | |
| | AGACGAAGAAGCCCACAGGCGCCCCUCCAGAACUGUCUG | |
| | GCGUGACCAACAAUGGCUUCAUCCCUCACAGCUGAUGAG | |
| | CUCGCUUUCUUGCUGUCCAAUUUCUAUUAAAGGUUCCUU | |
| | UGUUCCCUAAGUCCAACUACUAAACUGGGGGAUAUUAUG | |
| | AAGGGCCUUGAGCAUCUGGAUUCUGCCUAAUAAAAAACA | |
| | UUUAUUUUCAUUGCAAaaaaaaaaaaaaaaaaaaaaaaa | |
| | aaaaaaaaaaaaaaaaaaaaaaaaaaaaaaaaaaaaaaa | |
| | aaaaaaaaaaaaaaaaaa | |
| hMPV198_5UTR_582/ | AGGUGUCAGAGUUUAACUUGAAGACUAUUUCUAGGGAUA | 667 |
| 3UTR_2 80A_modRNA | AUAUCCCUGCCACCAUGAGCUGGAAGGUGGUCAUCAUCU | |
| Underline = 5′ cap; | UCAGCCUGCUGAUCACCCCUCAGCACGGCCUGAAAGAGA | |
| bold = 5′UTR | GCUACCUGGAAGAAAGCUGCAGCACCAUCACCGAGGGCU | |
| (5UTR_582) and | ACCUGAGCGUGCUGAGAACCGGCUGGUACACCAACGUGU | |
| 3′ UTR( 3UTR_2); | UCACCCUGGAAGUGGGCGACGUGGAAAACCUGACCUGUG | |
| italics = KOZAK | CCGAUGGACCCAGCCUGAUCAAGACCGAGCUGGACCUGA | |
| sequence; | CAAAGAGCGCCCUGCGGGAACUGAAAACCUGCUCUGCAG | |
| lowercase = | AUCAGGGCAGCGGCGGCAGCGGCGCUACAGCUGCUGCUG | |
| polyA tail | UGACAGCUGGCGUGGCCAUUGCCAAGACCAUCCGGCUGG | |
| (Amino acid | AAAGCGAAGUGACCGCCAUCAAGAACUGCCUGAAAAAGA | |
| SEQ ID NO: 643) | CCAACGAGUGCGUGUCUACCCUCGGCAAUGGCGUUAGAG | |
| | UGCUGGCCACAGCCGUCCGCGAGCUGAAGGAUUUCGUGU | |
| | CCAAGAACCUGACCAGGGCCAUCAACAAGAACAAGUGCG | |
| | ACAUCCCCGACCUGAAGAUGGCCGUGUCCUUCAGCCAGU | |
| | UCAACCGGCGGUUCCUGAAUGUCGUGCGGCAGUUCUCUG | |
| | ACAACGCCGGCAUCACACCAGCCAUCAGCCUGGAUCUGA | |
| | UGACCGAUGCCGAACUGGCUAGAGCCGUGUCCAACAUGC | |
| | CUACAUCUGCCGGCCAGAUCAAGCUGAUGCUGGAAAACA | |
| | GAUGCAUGGUCCGACGGAAAGGCUUCGGCAUCCUGAUCG | |
| | GCGUGUACGGCAGCAGCGUGAUCUACAUGGUGCAGCUGC | |
| | CUAUCUUCGGCGUGAUCGACACCCCUUGCUGGAUCGUGA | |
| | AAGCCGCUCCUAGCUGCAGCGAGAAGAAGGGCAAUUACG | |
| | CCUGCCUGCUGAGAGAGGACCAAGGCUGGUACUGUCAGA | |
| | AUGCCGGCAGCACCGUGUACUACCCCAACGAGAAGGACU | |
| | GCGAGACAAGAGGCGACCACGUGUUCUGUGAUACCGCCG | |
| | CUGGAAUCAACGUGGCCGAGCAGAGCAAAGAGUGCAACA | |
| | UCAACAUCAGCACCACAAACUACCCCUGCAAGGUGUCCA | |
| | CCGGCAGACACCCUAUCAGCAUGGUGGCUCUGUCUCCAC | |
| | UGGGAGCCCUGGUGGCUUGUUAUAAGGGCGUGUCCUGUA | |
| | GCAUCGGCAGCAACAGAGUGGGCAUCAUCAAGCAGCUGA | |
| | ACAAGGGCUGCUCCUACAUCACCAACCAGGACGCCGAUA | |
| | CCGUGACCAUCGACAAUACCGUGUAUCAGCUGAGCAAGG | |
| | UGGAAGGCGAACAGCACGUGAUCAAGGGCAGACCUGUGU | |
| | CCAGCAGCUUCGACCCCGUGAAGUUCCCCGAGUGCCAGU | |
| | UCAAUUGCGCCCUGGACCAGGUGUUCGAGAACAUCGAGA | |
| | ACUCUCAGGCUCUGGUGGACCAGUCCAACCGGAUUCUGU | |
| | CUAGCGCCGAGAAGGGAAACACCGGCUUCAUCAUCGUGA | |
| | UCAUCCUGAUUGCCGUGCUGGGCUCCAGCAUGAUCCUGG | |
| | UGUCCAUCUUUAUCAUCAUCAAAAAGACGAAGAAGCCCA | |
| | CAGGCGCCCCUCCAGAACUGUCUGGCGUGACCAACAAUG | |
| | GCUUCAUCCCUCACAGCUGAUGAGCUCGCUUUCUUGCUG | |
| | UCCAAUUUCUAUUAAAGGUUCCUUUGUUCCCUAAGUCCA | |
| | ACUACUAAACUGGGGGAUAUUAUGAAGGGCCUUGAGCAU | |
| | CUGGAUUCUGCCUAAUAAAAAACAUUUAUUUUCAUUGCA | |
| | Aaaaaaaaaaaaaaaaaaaaaaaaaaaaaaaaaaaaaaa | |
| | aaaaaaaaaaaaaaaaaaaaaaaaaaaaaaaaaaaaaaa | |
| | aaa | |
| hMPV189_5UTR_582/ | AGGUGUCAGAGUUUAACUUGAAGACUAUUUCUAGGGAUA | 668 |
| 3UTR_2 80A_modRNA | AUAUCCCUGCCACCAUGAGCUGGAAAGUCAUGAUCAUCA | |
| Underline = 5′ cap; | UCAGCCUGCUGAUCACCCCUCAGCACGGCCUGAAAGAGA | |
| bold = 5′UTR | GCUACCUGGAAGAAAGCUGCAGCACCAUCACCGAGGGCU | |
| (5UTR_582) and | ACCUGAGCGUGCUGAGAACCGGCUGGUACACCAACGUGU | |
| 3′ UTR (3UTR_2); | UCACCCUGGAAGUGGGCGACGUGGAAAACCUGACCUGCA | |
| italics = KOZAK | CAGAUGGCCCCAGCCUGAUCAAGACCGAGCUGGAUCUGA | |
| sequence; | CAAAGAGCGCCCUGCGCGAGCUGAAAACCGUGUCUGCAG | |
| lowercase = | AUCAGCUGGCCAGAGAGGAACAGAUCGAGAACCCCAGAC | |
| polyA tail | GGCGGAGAUUCGUGCUGGGAGCUAUCGCCUGCGGAGUUG | |
| (Amino acid | CUACAGCUGCUGCUGUGACAGCCGGAAUCGCCAUUGCCA | |
| SEQ ID NO: 644) | AGUGCAUCCGGCUGGAAAGCGAAGUGAACGCCAUCAAGG | |
| | GCUGCCUGAAAACCACCAACGAGUGCGUGUCUACCCUCG | |
| | GCUGCGGUGUUAGAGUGCUGGCCACAGCCGUGCGGGAAC | |
| | UGAAAGAAUUCGUGUCCAAGAACCUGACCAGCGCCAUCA | |
| | ACAAGAACAAGUGCGACAUUCCCGACCUGAAGAUGGCCG | |
| | UGUCCUUCAGCCAGUUCAACCGGCGGUUCCUGAAUGUCG | |
| | UGCGGCAGUUCUCUGACAACGCCGGCAUCACACCAGCCA | |
| | UUAGCAAGGACCUGAUGAACGACGCCGAACUGGCUAGAG | |
| | CCAUCUCUUACAUGCCUACCUCUGCCGGCCAGAUCAAGC | |
| | UGAUGCUGGAAAACAGAGCCAUGGUCCGACGGAAAGGCU | |
| | UCGGCAUCCUGAUCGGCGUGUACGGCAGCAGCGUGAUCU | |
| | ACAUGGUGCAGCUGCCUAUCUUCGGCGUGAUCAACACCC | |
| | CUUGCUGGAUCAUCAAGGCCGCUCCUAGCUGCAGCGAGA | |
| | AGGACGGCAAUUACGCCUGCCUGCUGAGAGAGGACCAAG | |
| | GCUGGUACUGCAAGAAUGCCGGCAGCACCGUGUACUACC | |
| | CCUGCGAGAAGGAUUGCGAGACACGGGGCGAUCACGUGU | |
| | UCUGUGAUACAGCCGCCGGAAUCAACGUGGCCGAGCAGA | |
| | GCAGAGAGUGCAACAUCAACAUCAGCACCACAAACUACC | |
| | CCUGCAAGGUGUCCUGCGGCAGACACCCUAUCAGCAUGG | |
| | UGGCUCUGUCUCCACUGGGAGCCCUGGUGGCUUGUUAUA | |
| | AGGGCGUGUCCUGUAGCAUCGGCAGCAAUCAAGUGGGCA | |
| | UCAUCAAGCAGCUGCCCAAGGGCUGCUCCUACAUCACCA | |
| | AUCAGGACGCCGACACCGUGACCAUCGACAAUACCGUGU | |
| | AUCAGCUGAGCAAGGUGGAAGGCGAACAGCACGUGAUCA | |
| | AGGGCAGACCUGUGUCCAACAGCUUCGACCCCAUCAGAU | |
| | UCCCCCAGGACCAGUUCAAUGUGGCCCUGGACCAGUGCU | |
| | UCGAGAGCAUCGAGAAUAGCCAGGCUCUGGUGGACCAGU | |
| | CCAACAAGAUCCUGAACUCCGCCGAGAAGGGCAACACCG | |
| | GCUUCAUCAUCGUGAUCAUCCUGAUUGCCGUGCUGGGCC | |
| | UGACCAUGAUCAGCGUGUCCAUCAUCAUUAUCAUCAAGA | |
| | AAACGCGGAAGCCCGCCGGCGCCCCUCCAGAACUUAAUG | |
| | GCGUGACCAACGGCGGCUUCAUUCCCCACUCUUGAUGAG | |
| | CUCGCUUUCUUGCUGUCCAAUUUCUAUUAAAGGUUCCUU | |
| | UGUUCCCUAAGUCCAACUACUAAACUGGGGGAUAUUAUG | |
| | AAGGGCCUUGAGCAUCUGGAUUCUGCCUAAUAAAAAACA | |
| | UUUAUUUUCAUUGCAAaaaaaaaaaaaaaaaaaaaaaaa | |
| | aaaaaaaaaaaaaaaaaaaaaaaaaaaaaaaaaaaaaaa | |
| | aaaaaaaaaaaaaaaaaa | |
| hMPV194_5UTR_582/ | AGGUGUCAGAGUUUAACUUGAAGACUAUUUCUAGGGAUA | 669 |
| 3UTR_2 80A_modRNA | AUAUCCCUGCCACCAUGAGCUGGAAAGUCAUGAUCAUCA | |
| Underline = 5′ cap; | UCAGCCUGCUGAUCACCCCUCAGCACGGCCUGAAAGAGA | |
| bold = 5′UTR | GCUACCUGGAAGAAAGCUGCAGCACCAUCACCGAGGGCU | |
| (5UTR_582) and | ACCUGAGCGUGCUGAGAACCGGCUGGUACACCAACGUGA | |
| 3′ UTR (3UTR_2); | CAGAUGGCCCCAGCCUGAUCAAGACCGAGCUGGAUCUGA | |
| italics = KOZAK | CAAAGAGCGCCCUGCGCGAGCUGAAAACCUGCUCUGCAA | |
| sequence; | GUGCUGGCCACAGCCGUGCGGGAACUGAAAGAAUUCGUG | |
| lowercase = | UCCAAGAACCUGACCAGCGCCAUCAACAAGAACAAGUGG | |
| polyA tail | UUCAACCGGCGGUUCCUGAAUGUCGUGCGGCAGUUCUCA | |
| (Amino acid | CCAACGAGUGCGUGUCUACCCUCGGCAACGGUGUUAGCG | |
| SEQ ID NO: 645) | ACAUUCCCGACCUGAAGAUGGCCGUGUCCUUCAGCCAGA | |
| | UCAGGGCAGCGGCGGCAGCGGCGCUACAGCUGCUGCUGU | |
| | GACAGCCGGAAUCGCCAUUGCCAAGACCAUCCGGCUGGA | |
| | AAGCGAAGUGAACGCCAUCAAGGGCUGCCUGAAAACUUC | |
| | ACCCUGGAAGUGGGCGACGUGGAAAACCUGACCUGCCUG | |
| | ACAACGCCGGCAUCACACCAGCCAUUAGCCUGGACCUGA | |
| | UGAACGACGCCGAACUGGCUAGAGCCGUGUCUUACAUGC | |
| | CUACCUCUGCCGGCCAGAUCAAGCUGAUGCUGGAAAACA | |
| | GAUGCAUGGUCCGACGGAAAGGCUUCGGCAUCCUGAUCG | |
| | GCGUGUACGGCAGCAGCGUGAUCUACAUGGUGCAGCUGC | |
| | CUAUCUUCGGCGUGAUCAACACCCCUUGCUGGAUCAUCA | |
| | AGGCCGCUCCUAGCUGCAGCGAGAAGGACGGCAAUUACG | |
| | CCUGCCUGCUGAGAGAGGACCAAGGCUGGUACUGCAAGA | |
| | AUGCCGGCAGCACCGUGUACUACCCCAACGAGAAGGAUU | |
| | GCGAGACACGGGGCGAUCACGUGUUCUGUGAUACAGCCG | |
| | CCGGAAUCAACGUGGCCGAGCAGAGCAGAGAGUGCAACA | |
| | UCAACAUCAGCACCACAAACUACCCCUGCAAGGUGUCCA | |
| | CCGGCAGACACCCUAUCAGCAUGGUGGCUCUGUCUCCAC | |
| | UGGGAGCCCUGGUGGCUUGUUAUAAGGGCGUGUCCUGUA | |
| | GCAUCGGCAGCAAUCAAGUGGGCAUCAUCAAGCAGCUGC | |
| | CCAAGGGCUGCUCCUACAUCACCAAUCAGGACGCCGACA | |
| | CCGUGACCAUCGACAAUACCGUGUAUCAGCUGAGCAAGG | |
| | UGGAAGGCGAACAGCACGUGAUCAAGGGCAGACCUGUGU | |
| | CCAACAGCUUCGACCCCAUCAGAUUCCCCGAGUGCCAGU | |
| | UCAAUUGCGCCCUGGACCAGGUGUUCGAGAGCAUCGAGA | |
| | AUAGCCAGGCUCUGGUGGACCAGUCCAACAAGAUCCUGA | |
| | ACUCCGCCGAGAAGGGCAACACCGGCUUCAUCAUCGUGA | |
| | UCAUCCUGAUUGCCGUGCUGGGCCUGACCAUGAUCAGCG | |
| | UGUCCAUCAUCAUUAUCAUCAAGAAAACGCGGAAGCCCG | |
| | CCGGCGCCCCUCCAGAACUUAAUGGCGUGACCAACGGCG | |
| | GCUUCAUUCCCCACUCUUGAUGAGCUCGCUUUCUUGCUG | |
| | UCCAAUUUCUAUUAAAGGUUCCUUUGUUCCCUAAGUCCA | |
| | ACUACUAAACUGGGGGAUAUUAUGAAGGGCCUUGAGCAU | |
| | CUGGAUUCUGCCUAAUAAAAAACAUUUAUUUUCAUUGCA | |
| | Aaaaaaaaaaaaaaaaaaaaaaaaaaaaaaaaaaaaaaa | |
| | aaaaaaaaaaaaaaaaaaaaaaaaaaaaaaaaaaaaaaa | |
| | aaa | |

### C. PIV1 Sequences

TABLE 7
PIV1 F Polypeptides

| ID | SEQ ID NO: |
|---|---|
| Full Length F0 of PIV1 (GenBank GI: AFP49460.1) | 672 |
| PIV1 F PIV1047 | 673 |
| PIV1 F PIV1069 | 674 |
| PIV1 F PIV1047 Ecto-Foldon | 675 |
| PIV1 F PIV1069 Ecto-Foldon | 676 |

TABLE 8
PIV1 F DNA

| | SEQ |
|---|---|
| ID | ID NO: |
| PIV1 WT F | 677 |
| TGA stop codon; (Amino acid SEQ ID NO: 672) | |
| PIV1 F PIV1047 | 678 |
| TGA stop codon; (Amino acid SEQ ID NO: 673) | |
| PIV1 F PIV1069 | 679 |
| TGA stop codon; (Amino acid SEQ ID NO: 674) | |
| PIV1 F PIV1047 Ecto-Foldon | 682 |
| TGA stop codon; (Amino acid SEQ ID NO: 675) | |
| PIV1 F PIV1069 Ecto-Foldon | 683 |
| TGA stop codon; (Amino acid SEQ ID NO: 676) | |

| | | SEQ ID |
|---|---|---|
| ID | PIV1 Sequence | NO: |
| PIV1047_5UTR576/ | AGGAGGACTGGTCGAACCTGCATAGTGATCATAAGGTCAG | 680 |
| 3UTR_2 80A | CATAGCCACCATGCAGAGCAGCGAGATCCTGCTGCTGGTG | |
| PIV1 F PIV1047 | TACAGCTCTCTGCTGCTGAGCAGCTCCCTGTGTCAGATCC | |
| DNA | CCGTGGACAAGCTGAGCAACGTGGGCGTGATCATCAACGA | |
| Underline = 5′ cap; | GGGCAAGCTGCTGAAGATCGCCGGCAGCTACGAGAGCCGG | |
| bold = 5′ UTR | TACATCGTGCTGTCTCTGGTGCCCAGCATCGACCTGCAGG | |
| (5UTR_576) and | ATGGCTGTGGCACCACACAGATCATCCAGTACAAGAACCT | |
| 3′ UTR (3UTR_2); | GCTGAACCGGCTGCTGATCCCTCTGAAGGATGCCCTGGAT | |
| italics = KOZAK | CTGTGCGAGAGCCTGATCACCATCACCAACGACACCACCG | |
| sequence; | TGACCAACGATAACCCTCAGACCAGAGGCAGCGGCGCCGT | |
| lowercase = | GATCGGAACAATCGCCCTGGGAGTTGCTACCGCCGCTCAG | |
| polyA tail | ATCACAGCCTGCATTGCTCTGGCCGAAGCCAGAGAGGCCA | |
| (Amino acid | GAAAGGATATCGCCCTGATCAAGGACAGCATCGTGAAAAC | |
| SEQ ID NO: 673) | CCACAACAGCGTGGAATTCATCCAGCGCGGCATCGGCGAG | |
| | CAGATCATTGCCCTGAAAACCCTGCAGGACTTCGTGAACG | |
| | ACGAGATCAGACCCGCCATCGGAGAGCTGAGATGCGAGAC | |
| | AACAGCCCTGAAGCTGGGCATCAAGCTGACCCAGCACTAC | |
| | AGCGAACTGGCCACCGCCTTTAGCAGCAACCTGGGCACAA | |
| | TCGGCGAGAAGTCCCTGACACTGCAGGCCCTGAGCAGCCT | |
| | GTACAGCGCCAATATCACAGAGATCCTGTCCACCATCAAG | |
| | AAGGACAAGAGCGACATCTACGACATCATCTACACCGAGC | |
| | AAGTGAAGGGCACCGTGATCGACGTGGACCTGGAAAAGTA | |
| | CATGGTCACCCTGCTGGTCAAGATCCCCATCCTGTCTGAG | |
| | ATCCCTGGCGTGCTGATCTACAGAGCCAGCAGCATCAGCT | |
| | ACAACATCGAGGGCGAAGAATGGCACGTGGCAATCCCCAA | |
| | CTACATCATCAACAAGGCCAGCAGCCTCGGCGGAGCCGAT | |
| | GTGACAAATTGCATCGAGAGCAAGCTGGCCTACATCTGCC | |
| | CCAGAGATCCCACACAGCTGATCCCCGACAACCAGCAGAA | |
| | GTGCATCCTGGGCGACGTGTCCAAGTGTCCTGTGACCAAA | |
| | GTGATCAACAACCTGGTGCCTAAGTTCGCCTTCATCAACG | |
| | GCGGCGTGGTGGCCAACTGTATCGCCAGCACATGCACATG | |
| | CGGCACCAACAGAATCCCCGTGAACCAGGACAGATCCAAG | |
| | GGCGTGACCTTCCTGACCTACACCAACTGTGGCCTGATCG | |
| | GCATCAATGGCATCGAGCTGTACGCCAACAAGCGGGGCAG | |
| | AGATACCACCTGGGGCAACCAGATCATCAAAGTGGGCCCT | |
| | GCCGTGTCCATCCGGCCTGTGGATATCTCTCTGAACCTGG | |
| | CCAGCCTGACCAACTTTCTGGAAGAACTGAAGACCGAACT | |
| | CATGAAGCTGAGAGCCATCATCTCTGCCGTTGGCGGCTGG | |
| | CACAACAAAGAGAGCACCCAGATCATTATCATCATCATCG | |
| | TCTGCATCCTGATCATCATTATCTGCGGCATCCTGTACTA | |
| | CCTGTACAGAGTGCGGAGACTGCTCATCATGATCAACAGC | |
| | ACCAACAATAGCCCCATCAACGCCTACACACTGGAAAGCC | |
| | GGATGAAGAACCCCTACATGGGCAACCACAGCAACTGATG | |
| | AGCTCGCTTTCTTGCTGTCCAATTTCTATTAAAGGTTCCT | |
| | TTGTTCCCTAAGTCCAACTACTAAACTGGGGGATATTATG | |
| | AAGGGCCTTGAGCATCTGGATTCTGCCTAATAAAAAACAT | |
| | TTATTTTCATTGCAAaaaaaaaaaaaaaaaaaaaaaaaaa | |
| | aaaaaaaaaaaaaaaaaaaaaaaaaaaaaaaaaaaaaaaa | |
| | aaaaaaaaaaaaaaa | |
| PIV1069_5UTR_576/ | AGGAGGACTGGTCGAACCTGCATAGTGATCATAAGGTCAG | 681 |
| 3UTR_2 80A | CATAGCCACCATGCAGAGCAGCGAGATCCTGCTGCTGGTG | |
| PIV1 F PIV1069 | TACAGCTCTCTGCTGCTGAGCAGCTCCCTGTGTCAGATCC | |
| Underline = 5′ cap; | CCGTGGACAAGCTGAGCAACGTGGGCGTGATCATCAACGA | |
| bold = 5′UTR | GGGCAAGCTGCTGAAGATCGCCGGCAGCTACGAGAGCCGG | |
| (5UTR_576) and | TACATCGTGCTGTCTCTGGTGCCCAGCATCGACCTGCAGG | |
| 3′ UTR (3UTR_2); | ATGGCTGTGGCACCACACAGATCATCCAGTACAAGAACCT | |
| italics = KOZAK | GCTGAACCGGCTGCTGATCCCTCTGAAGGATGCCCTGGAT | |
| sequence; | CTGCAAGAGAGCCTGATCACCATCACCAACGACACCACCG | |
| lowercase = | TGACCAACGATAACCCTCAGACCAGAGGCAGCGGCGCCGT | |
| polyA tail | GATCGGAACAATCGCCCTGGGAGTTGCTACCGCCGCTCAG | |
| (Amino acid | ATCACAGCCGCCATTGCTCTGGCCGAAGCCAGAGAGGCCA | |
| SEQ ID NO: 674) | GAAAGGATATCGCCCTGATCAAGGACAGCATCGTGAAAAC | |
| | CCACAACAGCGTGGAATTCATCCAGCGCGGCATCGGCGAG | |
| | CAGATCATTGCCCTGAAAACCCTGCAGGACTTCGTGAACG | |
| | ACGAGATCAGACCCGCCATCGGAGAGCTGAGATGCGAGAC | |
| | AACAGCCCTGAAGCTGGGCATCAAGCTGACCCAGCACTAC | |
| | AGCGAACTGGCCACCGCCTTTAGCAGCAACCTGGGCACAA | |
| | TCGGCGAGAAGTCCCTGACACTGCAGGCCCTGAGCAGCCT | |
| | GTACAGCGCCAATATCACAGAGATCCTGTCCACCATCAAG | |
| | AAGGACAAGAGCGACATCTACGACATCATCTACACCGAGC | |
| | AAGTGAAGGGCACCGTGATCGACGTGGACCTGGAAAAGTA | |
| | CATGGTCACCCTGCTGGTCAAGATCCCCATCCTGTCTGAG | |
| | ATCCCTGGCGTGCTGATCTACAGAGCCAGCAGCATCAGCT | |
| | ACAACATCGAGGGCGAAGAATGGCACGTGGCAATCCCCAA | |
| | CTACATCATCAACAAGGCCAGCAGCCTCGGCGGAGCCGAT | |
| | GTGACAAATTGCATCGAGAGCAAGCTGGCCTACATCTGCC | |
| | CCAGAGATCCCACACAGCTGATCCCCGACAACCAGCAGAA | |
| | GTGCATCCTGGGCGACGTGTCCAAGTGTCCTGTGACCAAA | |
| | GTGATCAACAACCTGGTGCCTAAGTTCGCCTTCATCAACG | |
| | GCGGCGTGGTGGCCAACTGTATCGCCAGCACATGCACATG | |
| | CGGCACCAACAGAATCCCCGTGAACCAGGACAGATCCAAG | |
| | GGCGTGACCTTCCTGACCTACACCAACTGTGGCCTGATCG | |
| | GCATCAATGGCATCGAGCTGTACGCCAACAAGCGGGGCAG | |
| | AGATACCACCTGGGGCAACCAGATCATCAAAGTGGGCCCT | |
| | GCCGTGTCCATCCGGCCTGTGGATATCTCTCTGAACCTGG | |
| | CCAGCCTGACCAACTTTCTGGAAGAACTGAAGACCGAACT | |
| | CATGAAGGCCAGAGCCATCATCTCTGCCGTTGGCGGCTGG | |
| | CACAACAAAGAGAGCACCCAGATCATTATCATCATCATCG | |
| | TCTGCATCCTGATCATCATTATCTGCGGCATCCTGTACTA | |
| | CCTGTACAGAGTGCGGAGACTGCTCATCATGATCAACAGC | |
| | ACCAACAATAGCCCCATCAACGCCTACACACTGGAAAGCC | |
| | GGATGAAGAACCCCTACATGGGCAACCACAGCAACTGATG | |
| | AGCTCGCTTTCTTGCTGTCCAATTTCTATTAAAGGTTCCT | |
| | TTGTTCCCTAAGTCCAACTACTAAACTGGGGGATATTATG | |
| | AAGGGCCTTGAGCATCTGGATTCTGCCTAATAAAAAACAT | |
| | TTATTTTCATTGCAAaaaaaaaaaaaaaaaaaaaaaaaaa | |
| | aaaaaaaaaaaaaaaaaaaaaaaaaaaaaaaaaaaaaaaa | |
| | aaaaaaaaaaaaaaa | |

TABLE 9
PIV1 F RNA

| | SEQ ID |
|---|---|
| ID | NO: |
| PIV1 WT F | 767 |
| UGA stop codon; (Amino acid SEQ ID NO: 672) | |
| PIV1 F PIV1047 | 684 |
| UGA stop codon; (Amino acid SEQ ID NO: 673) | |
| PIV1 F PIV1069 | 685 |
| UGA stop codon; (Amino acid SEQ ID NO: 674) | |
| PIV1 F PIV1047 Ecto-Foldon | 688 |
| UGA stop codon; (Amino acid SEQ ID NO: 675) | |
| PIV1 F PIV1069 Ecto-Foldon | 689 |
| UGA stop codon; (Amino acid SEQ ID NO: 676) | |

| | | SEQ ID |
|---|---|---|
| ID | Sequence | NO: |
| PIV1047_5UTR576/ | AGGAGGACUGGUCGAACCUGCAUAGUGAUCAUAAGGUC | 686 |
| 3UTR_2 80A_modRNA | AGCAUAGCCACCAUGCAGAGCAGCGAGAUCCUGCUGCU | |
| Underline = 5′ cap; | GGUGUACAGCUCUCUGCUGCUGAGCAGCUCCCUGUGUC | |
| bold = 5′ UTR | AGAUCCCCGUGGACAAGCUGAGCAACGUGGGCGUGAUC | |
| (5UTR_576) and | AUCAACGAGGGCAAGCUGCUGAAGAUCGCCGGCAGCUA | |
| 3′ UTR (3UTR_2); | CGAGAGCCGGUACAUCGUGCUGUCUCUGGUGCCCAGCA | |
| italics = KOZAK | UCGACCUGCAGGAUGGCUGUGGCACCACACAGAUCAUC | |
| sequence; | CAGUACAAGAACCUGCUGAACCGGCUGCUGAUCCCUCU | |
| lowercase = | GAAGGAUGCCCUGGAUCUGUGCGAGAGCCUGAUCACCA | |
| polyA tail | UCACCAACGACACCACCGUGACCAACGAUAACCCUCAG | |
| (Amino acid | ACCAGAGGCAGCGGCGCCGUGAUCGGAACAAUCGCCCU | |
| SEQ ID NO: 673) | GGGAGUUGCUACCGCCGCUCAGAUCACAGCCUGCAUUG | |
| | CUCUGGCCGAAGCCAGAGAGGCCAGAAAGGAUAUCGCC | |
| | CUGAUCAAGGACAGCAUCGUGAAAACCCACAACAGCGU | |
| | GGAAUUCAUCCAGCGCGGCAUCGGCGAGCAGAUCAUUG | |
| | CCCUGAAAACCCUGCAGGACUUCGUGAACGACGAGAUC | |
| | AGACCCGCCAUCGGAGAGCUGAGAUGCGAGACAACAGC | |
| | CCUGAAGCUGGGCAUCAAGCUGACCCAGCACUACAGCG | |
| | AACUGGCCACCGCCUUUAGCAGCAACCUGGGCACAAUC | |
| | GGCGAGAAGUCCCUGACACUGCAGGCCCUGAGCAGCCU | |
| | GUACAGCGCCAAUAUCACAGAGAUCCUGUCCACCAUCA | |
| | AGAAGGACAAGAGCGACAUCUACGACAUCAUCUACACC | |
| | GAGCAAGUGAAGGGCACCGUGAUCGACGUGGACCUGGA | |
| | AAAGUACAUGGUCACCCUGCUGGUCAAGAUCCCCAUCC | |
| | UGUCUGAGAUCCCUGGCGUGCUGAUCUACAGAGCCAGC | |
| | AGCAUCAGCUACAACAUCGAGGGCGAAGAAUGGCACGU | |
| | GGCAAUCCCCAACUACAUCAUCAACAAGGCCAGCAGCC | |
| | UCGGCGGAGCCGAUGUGACAAAUUGCAUCGAGAGCAAG | |
| | CUGGCCUACAUCUGCCCCAGAGAUCCCACACAGCUGAU | |
| | CCCCGACAACCAGCAGAAGUGCAUCCUGGGCGACGUGU | |
| | CCAAGUGUCCUGUGACCAAAGUGAUCAACAACCUGGUG | |
| | CCUAAGUUCGCCUUCAUCAACGGCGGCGUGGUGGCCAA | |
| | CUGUAUCGCCAGCACAUGCACAUGCGGCACCAACAGAA | |
| | UCCCCGUGAACCAGGACAGAUCCAAGGGCGUGACCUUC | |
| | CUGACCUACACCAACUGUGGCCUGAUCGGCAUCAAUGG | |
| | CAUCGAGCUGUACGCCAACAAGCGGGGCAGAGAUACCA | |
| | CCUGGGGCAACCAGAUCAUCAAAGUGGGCCCUGCCGUG | |
| | UCCAUCCGGCCUGUGGAUAUCUCUCUGAACCUGGCCAG | |
| | CCUGACCAACUUUCUGGAAGAACUGAAGACCGAACUCA | |
| | UGAAGCUGAGAGCCAUCAUCUCUGCCGUUGGCGGCUGG | |
| | CACAACAAAGAGAGCACCCAGAUCAUUAUCAUCAUCAU | |
| | CGUCUGCAUCCUGAUCAUCAUUAUCUGCGGCAUCCUGU | |
| | ACUACCUGUACAGAGUGCGGAGACUGCUCAUCAUGAUC | |
| | AACAGCACCAACAAUAGCCCCAUCAACGCCUACACACU | |
| | GGAAAGCCGGAUGAAGAACCCCUACAUGGGCAACCACA | |
| | GCAACUGAUGAGCUCGCUUUCUUGCUGUCCAAUUUCUA | |
| | UUAAAGGUUCCUUUGUUCCCUAAGUCCAACUACUAAAC | |
| | UGGGGGAUAUUAUGAAGGGCCUUGAGCAUCUGGAUUCU | |
| | GCCUAAUAAAAAACAUUUAUUUUCAUUGCAAaaaaaaa | |
| | aaaaaaaaaaaaaaaaaaaaaaaaaaaaaaaaaaaaaa | |
| | aaaaaaaaaaaaaaaaaaaaaaaaaaaaaaaaaaa | |
| PIV1069_5UTR576/ | AGGAGGACUGGUCGAACCUGCAUAGUGAUCAUAAGGUC | 687 |
| 3UTR_2 80A_modRNA | AGCAUAGCCACCAUGCAGAGCAGCGAGAUCCUGCUGCU | |
| Underline = 5′ cap; | GGUGUACAGCUCUCUGCUGCUGAGCAGCUCCCUGUGUC | |
| bold = 5′ UTR | AGAUCCCCGUGGACAAGCUGAGCAACGUGGGCGUGAUC | |
| (5UTR_576) and | AUCAACGAGGGCAAGCUGCUGAAGAUCGCCGGCAGCUA | |
| 3′ UTR (3UTR_2); | CGAGAGCCGGUACAUCGUGCUGUCUCUGGUGCCCAGCA | |
| italics = KOZAK | UCGACCUGCAGGAUGGCUGUGGCACCACACAGAUCAUC | |
| sequence; | CAGUACAAGAACCUGCUGAACCGGCUGCUGAUCCCUCU | |
| lowercase = | GAAGGAUGCCCUGGAUCUGCAAGAGAGCCUGAUCACCA | |
| polyA tail | UCACCAACGACACCACCGUGACCAACGAUAACCCUCAG | |
| (Amino acid | ACCAGAGGCAGCGGCGCCGUGAUCGGAACAAUCGCCCU | |
| SEQ ID NO: 674) | GGGAGUUGCUACCGCCGCUCAGAUCACAGCCGCCAUUG | |
| | CUCUGGCCGAAGCCAGAGAGGCCAGAAAGGAUAUCGCC | |
| | CUGAUCAAGGACAGCAUCGUGAAAACCCACAACAGCGU | |
| | GGAAUUCAUCCAGCGCGGCAUCGGCGAGCAGAUCAUUG | |
| | CCCUGAAAACCCUGCAGGACUUCGUGAACGACGAGAUC | |
| | AGACCCGCCAUCGGAGAGCUGAGAUGCGAGACAACAGC | |
| | CCUGAAGCUGGGCAUCAAGCUGACCCAGCACUACAGCG | |
| | AACUGGCCACCGCCUUUAGCAGCAACCUGGGCACAAUC | |
| | GGCGAGAAGUCCCUGACACUGCAGGCCCUGAGCAGCCU | |
| | GUACAGCGCCAAUAUCACAGAGAUCCUGUCCACCAUCA | |
| | AGAAGGACAAGAGCGACAUCUACGACAUCAUCUACACC | |
| | GAGCAAGUGAAGGGCACCGUGAUCGACGUGGACCUGGA | |
| | AAAGUACAUGGUCACCCUGCUGGUCAAGAUCCCCAUCC | |
| | UGUCUGAGAUCCCUGGCGUGCUGAUCUACAGAGCCAGC | |
| | AGCAUCAGCUACAACAUCGAGGGCGAAGAAUGGCACGU | |
| | GGCAAUCCCCAACUACAUCAUCAACAAGGCCAGCAGCC | |
| | UCGGCGGAGCCGAUGUGACAAAUUGCAUCGAGAGCAAG | |
| | CUGGCCUACAUCUGCCCCAGAGAUCCCACACAGCUGAU | |
| | CCCCGACAACCAGCAGAAGUGCAUCCUGGGCGACGUGU | |
| | CCAAGUGUCCUGUGACCAAAGUGAUCAACAACCUGGUG | |
| | CCUAAGUUCGCCUUCAUCAACGGCGGCGUGGUGGCCAA | |
| | CUGUAUCGCCAGCACAUGCACAUGCGGCACCAACAGAA | |
| | UCCCCGUGAACCAGGACAGAUCCAAGGGCGUGACCUUC | |
| | CUGACCUACACCAACUGUGGCCUGAUCGGCAUCAAUGG | |
| | CAUCGAGCUGUACGCCAACAAGCGGGGCAGAGAUACCA | |
| | CCUGGGGCAACCAGAUCAUCAAAGUGGGCCCUGCCGUG | |
| | UCCAUCCGGCCUGUGGAUAUCUCUCUGAACCUGGCCAG | |
| | CCUGACCAACUUUCUGGAAGAACUGAAGACCGAACUCA | |
| | UGAAGGCCAGAGCCAUCAUCUCUGCCGUUGGCGGCUGG | |
| | CACAACAAAGAGAGCACCCAGAUCAUUAUCAUCAUCAU | |
| | CGUCUGCAUCCUGAUCAUCAUUAUCUGCGGCAUCCUGU | |
| | ACUACCUGUACAGAGUGCGGAGACUGCUCAUCAUGAUC | |
| | AACAGCACCAACAAUAGCCCCAUCAACGCCUACACACU | |
| | GGAAAGCCGGAUGAAGAACCCCUACAUGGGCAACCACA | |
| | GCAACUGAUGAGCUCGCUUUCUUGCUGUCCAAUUUCUA | |
| | UUAAAGGUUCCUUUGUUCCCUAAGUCCAACUACUAAAC | |
| | UGGGGGAUAUUAUGAAGGGCCUUGAGCAUCUGGAUUCU | |
| | GCCUAAUAAAAAACAUUUAUUUUCAUUGCAAaaaaaaa | |
| | aaaaaaaaaaaaaaaaaaaaaaaaaaaaaaaaaaaaaa | |
| | aaaaaaaaaaaaaaaaaaaaaaaaaaaaaaaaaaa | |

TABLE 10
PIV1 HN Polypeptides

| ID | SEQ ID NO: |
|---|---|
| Full Length PIV1 HN WT PIV1083 | 750 |
| (GenBank GI: ATI99865.1) | |
| (Reference sequence) | |
| Full Length PIV1 HN WT | 751 |
| (GenBank GI: AAA46845.1) | |
| Full Length PIV1 HN WT | 752 |
| (GenBank GI: AFP49353.1) | |
| Full Length PIV1 HN WT | 753 |
| (GenBank GI: BAK09330.1) | |
| Full Length PIV1 HN WT | 754 |
| (GenBank GI: AXR70621.1) | |
| Full Length PIV1 HN WT | 755 |
| (GenBank GI: AAC23946.1) | |
| MY-U370/12_PIV1 HN_GA_Hs_optimized_del57-84 | 756 |
| PIV1084 | |
| MY-U370/12_PIV1 HN_GA_Hs_optimized_del57-129 | 757 |
| PIV1085 | |

TABLE 11
PIV1 HN DNA

| | SEQ ID |
|---|---|
| ID | NO: |
| PIV1 WT HN PIV1083 | 758 |
| TGA stop codon; (Amino acid SEQ ID NO: 750) | |
| PIV1 HN PIV1084 | 759 |
| TGA stop codon; (Amino acid SEQ ID NO: 756) | |
| PIV1 HN PIV1085 | 760 |
| TGA stop codon; (Amino acid SEQ ID NO: 757) | |

SEQ ID

| ID | Sequence | NO: |
|---|---|---|
| PIV1083_5UTR_579/ | AGGAGGACTGGGCGAACCTGCATAGTGATCATAAGGTCATCA | 768 |
| 3UTR_2 80A | TAGCCACCATGGCCGAGAAGGGCAAGACCATCAGCAGCTACT | |
| PIV1 HN WT | GGTCCACCACCAGAAACGACAACAGCACCGTGAACACCCACA | |
| PIV1083 | TCAACACCCCAGCCGGCAGAACCTACATCTGGCTGCTGATCG | |
| DNA | CCACCACCATGCACGCTGCCCTGAGCCTGATCATCATGATCC | |
| Underline = 5′ cap; | TGTGCATCGACCTCATCATCAAGCAGGATACCTGCATGAAGA | |
| bold = 5′UTR | CCAACATCATGACCGTGTCCAGCGTGAACGAGAGCGCCAAGA | |
| (5UTR_579) and | CAATCAAAGAGACAATCACCGAGCTGATCCGGCAAGAAGTGA | |
| 3′ UTR (3UTR_2); | TCAGCCGGACCATCAACATCCAGTCCTCTGTGCAGAGCGGCA | |
| italics = KOZAK | TCCCCATCCTGCTGAACAAGCAGAGCAGAGATCTGACCCAGC | |
| sequence; | TGATCGAGAAGTCCTGCAACAAGCAAGAGCTGGCCCAGATCT | |
| lowercase = polyA | GCGAGAACACAATCGCCATTCACCACGCCGACGGCATCACCC | |
| tail | CTCTGGACCCTCACGATTTTTGGAGATGCCCTGTGGGCGAGC | |
| (Amino acid | CCCTGCTGAGCAACAACCCCAATATCAGCCTGCTGCCTGGAC | |
| SEQ ID NO: 750) | CTTCTCTGCTGAGCGGCAGCACAACAATCTCTGGCTGCGTCA | |
| | GACTGCCCAGCCTGTCTATCGGAGATGCCATCTACGCCTACA | |
| | GCAGCAACCTGATCACACAGGGCTGCGCCGACATCGGCAAGA | |
| | GCTATCAGGTTCTGCAGCTGGGCTACATCAGCCTGAACAGCG | |
| | ACATGTACCCCGATCTGAACCCCGTGATCTCCCACACCTACG | |
| | ACATCAACGACAACCGCAAGTCCTGCTCCGTGATCGCCGCTG | |
| | GCACAAGAGGATACCAGCTGTGTAGCCTGCCTACCGTGAACG | |
| | AAACCACCGACTACAGCTCCGAGGGCATCGAGGACCTGGTGT | |
| | TCGACATCCTGGACCTGAAGGGAAAGACAAAGAGCCACCGGT | |
| | ACAAGAACGAGGACATCACCTTCGATCACCCCTTCAGCGCCA | |
| | TGTATCCTAGCGTCGGCAGCGGCATCAAGATCGAGAACACCC | |
| | TGGTGTTTCTCGGCTACGGCGGCCTGACAACACCTCTGCAGG | |
| | GCAACACCAAATGCGTGATCAAGAGCTGCCCTAACGTGAACC | |
| | AGTCCGTGTGCAACGACGCCCTGAAGATCACCTGGCTGAAGA | |
| | AACGGCAGGTCGTGAACGTGCTGATCCACATTAACAACTACC | |
| | TGAGCGACAGGCCCAAGATCGTGGTGGAAACAATCCCCATCA | |
| | CACAGAACTACCTGGGCGCCGAAGGCAGACTGCTGAAGCTGG | |
| | GCAAGAAGATCTACATCTACACCAGAAGCAGCGGCTGGCACT | |
| | CCAACCTGCAGATCGGCAGCATCGATATCAACAAGCCCATGA | |
| | CCATCAATTGGGCCCCTCACAAGGTGCTGAGCAGACCCGGAA | |
| | ATCCCGACTGCAACTGGTTCAACAAGTGCCCCAGAGAATGCA | |
| | TCAGCGGCGTGTACACCGACGCCTATCCTCTGAGCCCCGACG | |
| | CCTTTAACGTGGCCACCACAACACTGTACGCCAACACCAGCA | |
| | GAGTGAACCCCACCATCATGTACAGCTCCACCAGCAAGATCA | |
| | TCAACATGCTGAGACTGAAGAACGGCCAGCTGGAAGCCGCCT | |
| | ACACCACCACAAGCTGCATCACCCACTTCGGCAAGGGCTACT | |
| | GCTTCCACATCGTGGAAATCAACCAGACCTCTCTGAACACAC | |
| | TGCAGCCCATGCTGTTCAAGACCAGCATTCCCAAGATCTGCA | |
| | AAGTGACCAGCTGATGAGCTCGCTTTCTTGCTGTCCAATTTC | |
| | TATTAAAGGTTCCTTTGTTCCCTAAGTCCAACTACTAAACTG | |
| | GGGGATATTATGAAGGGCCTTGAGCATCTGGATTCTGCCTAA | |
| | TAAAAAACATTTATTTTCATTGCAAaaaaaaaaaaaaaaaaa | |
| | aaaaaaaaaaaaaaaaaaaaaaaaaaaaaaaaaaaaaaaaaa | |
| | aaaaaaaaaaaaaaaaaaaaa | |
| PIV1084_5UTR_579/ | AGGAGGACTGGGCGAACCTGCATAGTGATCATAAGGTCATCA | 769 |
| 3UTR_2 80A | TAGCCACCATGGCCGAGAAGGGCAAGACCATCAGCAGCTACT | |
| PIV1 HN | GGTCCACCACCAGAAACGACAACAGCACCGTGAACACCCACA | |
| del57-84 | TCAACACCCCAGCCGGCAGAACCTACATCTGGCTGCTGATCG | |
| PIV1084 | CCACCACCATGCACGCTGCCCTGAGCCTGATCATCATGATCC | |
| DNA | TGTGCATCGAGACAATCACCGAGCTGATCCGGCAAGAAGTGA | |
| Underline = 5′ cap; | TCAGCCGGACCATCAACATCCAGTCCTCTGTGCAGAGCGGCA | |
| bold = 5′UTR | TCCCCATCCTGCTGAACAAGCAGAGCAGAGATCTGACCCAGC | |
| (5UTR_579) | TGATCGAGAAGTCCTGCAACAAGCAAGAGCTGGCCCAGATCT | |
| and | GCGAGAACACAATCGCCATTCACCACGCCGACGGCATCACCC | |
| 3′ UTR (3UTR_2); | CTCTGGACCCTCACGATTTTTGGAGATGCCCTGTGGGCGAGC | |
| italics = KOZAK | CCCTGCTGAGCAACAACCCCAATATCAGCCTGCTGCCTGGAC | |
| sequence; | CTTCTCTGCTGAGCGGCAGCACAACAATCTCTGGCTGCGTCA | |
| lowercase = | GACTGCCCAGCCTGTCTATCGGAGATGCCATCTACGCCTACA | |
| polyA tail | GCAGCAACCTGATCACACAGGGCTGCGCCGACATCGGCAAGA | |
| (Amino acid | GCTATCAGGTTCTGCAGCTGGGCTACATCAGCCTGAACAGCG | |
| SEQ ID NO: 756) | ACATGTACCCCGATCTGAACCCCGTGATCTCCCACACCTACG | |
| | ACATCAACGACAACCGCAAGTCCTGCTCCGTGATCGCCGCTG | |
| | GCACAAGAGGATACCAGCTGTGTAGCCTGCCTACCGTGAACG | |
| | AAACCACCGACTACAGCTCCGAGGGCATCGAGGACCTGGTGT | |
| | TCGACATCCTGGACCTGAAGGGAAAGACAAAGAGCCACCGGT | |
| | ACAAGAACGAGGACATCACCTTCGATCACCCCTTCAGCGCCA | |
| | TGTATCCTAGCGTCGGCAGCGGCATCAAGATCGAGAACACCC | |
| | TGGTGTTTCTCGGCTACGGCGGCCTGACAACACCTCTGCAGG | |
| | GCAACACCAAATGCGTGATCAAGAGCTGCCCTAACGTGAACC | |
| | AGTCCGTGTGCAACGACGCCCTGAAGATCACCTGGCTGAAGA | |
| | AACGGCAGGTCGTGAACGTGCTGATCCACATTAACAACTACC | |
| | TGAGCGACAGGCCCAAGATCGTGGTGGAAACAATCCCCATCA | |
| | CACAGAACTACCTGGGCGCCGAAGGCAGACTGCTGAAGCTGG | |
| | GCAAGAAGATCTACATCTACACCAGAAGCAGCGGCTGGCACT | |
| | CCAACCTGCAGATCGGCAGCATCGATATCAACAAGCCCATGA | |
| | CCATCAATTGGGCCCCTCACAAGGTGCTGAGCAGACCCGGAA | |
| | ATCCCGACTGCAACTGGTTCAACAAGTGCCCCAGAGAATGCA | |
| | TCAGCGGCGTGTACACCGACGCCTATCCTCTGAGCCCCGACG | |
| | CCTTTAACGTGGCCACCACAACACTGTACGCCAACACCAGCA | |
| | GAGTGAACCCCACCATCATGTACAGCTCCACCAGCAAGATCA | |
| | TCAACATGCTGAGACTGAAGAACGGCCAGCTGGAAGCCGCCT | |
| | ACACCACCACAAGCTGCATCACCCACTTCGGCAAGGGCTACT | |
| | GCTTCCACATCGTGGAAATCAACCAGACCTCTCTGAACACAC | |
| | TGCAGCCCATGCTGTTCAAGACCAGCATTCCCAAGATCTGCA | |
| | AAGTGACCAGCTGATGAGCTCGCTTTCTTGCTGTCCAATTTC | |
| | TATTAAAGGTTCCTTTGTTCCCTAAGTCCAACTACTAAACTG | |
| | GGGGATATTATGAAGGGCCTTGAGCATCTGGATTCTGCCTAA | |
| | TAAAAAACATTTATTTTCATTGCAAaaaaaaaaaaaaaaaaa | |
| | aaaaaaaaaaaaaaaaaaaaaaaaaaaaaaaaaaaaaaaaaa | |
| | aaaaaaaaaaaaaaaaaaaaa | |
| PIV1085_5UTR_579/ | AGGAGGACTGGGCGAACCTGCATAGTGATCATAAGGTCATCA | 770 |
| 3UTR_2 80A | TAGCCACCATGGCCGAGAAGGGCAAGACCATCAGCAGCTACT | |
| PIV1 HN | GGTCCACCACCAGAAACGACAACAGCACCGTGAACACCCACA | |
| del57-129 | TCAACACCCCAGCCGGCAGAACCTACATCTGGCTGCTGATCG | |
| PIV1085 | CCACCACCATGCACGCTGCCCTGAGCCTGATCATCATGATCC | |
| DNA | TGTGCATCAACAAGCAAGAGCTGGCCCAGATCTGCGAGAACA | |
| Underline = 5′ cap; | CAATCGCCATTCACCACGCCGACGGCATCACCCCTCTGGACC | |
| bold = 5′UTR | CTCACGATTTTTGGAGATGCCCTGTGGGCGAGCCCCTGCTGA | |
| (5UTR_579) and | GCAACAACCCCAATATCAGCCTGCTGCCTGGACCTTCTCTGC | |
| 3′ UTR (3UTR_2); | TGAGCGGCAGCACAACAATCTCTGGCTGCGTCAGACTGCCCA | |
| italics = KOZAK | GCCTGTCTATCGGAGATGCCATCTACGCCTACAGCAGCAACC | |
| sequence; | TGATCACACAGGGCTGCGCCGACATCGGCAAGAGCTATCAGG | |
| lowercase = | TTCTGCAGCTGGGCTACATCAGCCTGAACAGCGACATGTACC | |
| polyA tail | CCGATCTGAACCCCGTGATCTCCCACACCTACGACATCAACG | |
| (Amino acid | ACAACCGCAAGTCCTGCTCCGTGATCGCCGCTGGCACAAGAG | |
| SEQ ID NO: 757) | GATACCAGCTGTGTAGCCTGCCTACCGTGAACGAAACCACCG | |
| | ACTACAGCTCCGAGGGCATCGAGGACCTGGTGTTCGACATCC | |
| | TGGACCTGAAGGGAAAGACAAAGAGCCACCGGTACAAGAACG | |
| | AGGACATCACCTTCGATCACCCCTTCAGCGCCATGTATCCTA | |
| | GCGTCGGCAGCGGCATCAAGATCGAGAACACCCTGGTGTTTC | |
| | TCGGCTACGGCGGCCTGACAACACCTCTGCAGGGCAACACCA | |
| | AATGCGTGATCAAGAGCTGCCCTAACGTGAACCAGTCCGTGT | |
| | GCAACGACGCCCTGAAGATCACCTGGCTGAAGAAACGGCAGG | |
| | TCGTGAACGTGCTGATCCACATTAACAACTACCTGAGCGACA | |
| | GGCCCAAGATCGTGGTGGAAACAATCCCCATCACACAGAACT | |
| | ACCTGGGCGCCGAAGGCAGACTGCTGAAGCTGGGCAAGAAGA | |
| | TCTACATCTACACCAGAAGCAGCGGCTGGCACTCCAACCTGC | |
| | AGATCGGCAGCATCGATATCAACAAGCCCATGACCATCAATT | |
| | GGGCCCCTCACAAGGTGCTGAGCAGACCCGGAAATCCCGACT | |
| | GCAACTGGTTCAACAAGTGCCCCAGAGAATGCATCAGCGGCG | |
| | TGTACACCGACGCCTATCCTCTGAGCCCCGACGCCTTTAACG | |
| | TGGCCACCACAACACTGTACGCCAACACCAGCAGAGTGAACC | |
| | CCACCATCATGTACAGCTCCACCAGCAAGATCATCAACATGC | |
| | TGAGACTGAAGAACGGCCAGCTGGAAGCCGCCTACACCACCA | |
| | CAAGCTGCATCACCCACTTCGGCAAGGGCTACTGCTTCCACA | |
| | TCGTGGAAATCAACCAGACCTCTCTGAACACACTGCAGCCCA | |
| | TGCTGTTCAAGACCAGCATTCCCAAGATCTGCAAAGTGACCA | |
| | GCTGATGAGCTCGCTTTCTTGCTGTCCAATTTCTATTAAAGG | |
| | TTCCTTTGTTCCCTAAGTCCAACTACTAAACTGGGGGATATT | |
| | ATGAAGGGCCTTGAGCATCTGGATTCTGCCTAATAAAAAACA | |
| | TTTATTTTCATTGCAAaaaaaaaaaaaaaaaaaaaaaaaaaa | |
| | aaaaaaaaaaaaaaaaaaaaaaaaaaaaaaaaaaaaaaaaaa | |
| | aaaaaaaaaaaa | |
| PIV1083_5UTR_582/ | AGGTGTCAGAGTTTAACTTGAAGACTATTTCTAGGGATAATA | 771 |
| 3UTR_2 80A | TCCCTGCCACCATGGCCGAGAAGGGCAAGACCATCAGCAGCT | |
| PIV1 HN WT | ACTGGTCCACCACCAGAAACGACAACAGCACCGTGAACACCC | |
| PIV1083 | ACATCAACACCCCAGCCGGCAGAACCTACATCTGGCTGCTGA | |
| DNA | TCGCCACCACCATGCACGCTGCCCTGAGCCTGATCATCATGA | |
| Underline = 5′ cap; | TCCTGTGCATCGACCTCATCATCAAGCAGGATACCTGCATGA | |
| bold = 5′ UTR | AGACCAACATCATGACCGTGTCCAGCGTGAACGAGAGCGCCA | |
| (5UTR_582) and | AGACAATCAAAGAGACAATCACCGAGCTGATCCGGCAAGAAG | |
| 3′ UTR (3UTR_2); | TGATCAGCCGGACCATCAACATCCAGTCCTCTGTGCAGAGCG | |
| italics = KOZAK | GCATCCCCATCCTGCTGAACAAGCAGAGCAGAGATCTGACCC | |
| sequence; | AGCTGATCGAGAAGTCCTGCAACAAGCAAGAGCTGGCCCAGA | |
| lowercase = | TCTGCGAGAACACAATCGCCATTCACCACGCCGACGGCATCA | |
| polyA tail | CCCCTCTGGACCCTCACGATTTTTGGAGATGCCCTGTGGGCG | |
| (Amino acid | AGCCCCTGCTGAGCAACAACCCCAATATCAGCCTGCTGCCTG | |
| SEQ ID NO: 750) | GACCTTCTCTGCTGAGCGGCAGCACAACAATCTCTGGCTGCG | |
| | TCAGACTGCCCAGCCTGTCTATCGGAGATGCCATCTACGCCT | |
| | ACAGCAGCAACCTGATCACACAGGGCTGCGCCGACATCGGCA | |
| | AGAGCTATCAGGTTCTGCAGCTGGGCTACATCAGCCTGAACA | |
| | GCGACATGTACCCCGATCTGAACCCCGTGATCTCCCACACCT | |
| | ACGACATCAACGACAACCGCAAGTCCTGCTCCGTGATCGCCG | |
| | CTGGCACAAGAGGATACCAGCTGTGTAGCCTGCCTACCGTGA | |
| | ACGAAACCACCGACTACAGCTCCGAGGGCATCGAGGACCTGG | |
| | TGTTCGACATCCTGGACCTGAAGGGAAAGACAAAGAGCCACC | |
| | GGTACAAGAACGAGGACATCACCTTCGATCACCCCTTCAGCG | |
| | CCATGTATCCTAGCGTCGGCAGCGGCATCAAGATCGAGAACA | |
| | CCCTGGTGTTTCTCGGCTACGGCGGCCTGACAACACCTCTGC | |
| | AGGGCAACACCAAATGCGTGATCAAGAGCTGCCCTAACGTGA | |
| | ACCAGTCCGTGTGCAACGACGCCCTGAAGATCACCTGGCTGA | |
| | AGAAACGGCAGGTCGTGAACGTGCTGATCCACATTAACAACT | |
| | ACCTGAGCGACAGGCCCAAGATCGTGGTGGAAACAATCCCCA | |
| | TCACACAGAACTACCTGGGCGCCGAAGGCAGACTGCTGAAGC | |
| | TGGGCAAGAAGATCTACATCTACACCAGAAGCAGCGGCTGGC | |
| | ACTCCAACCTGCAGATCGGCAGCATCGATATCAACAAGCCCA | |
| | TGACCATCAATTGGGCCCCTCACAAGGTGCTGAGCAGACCCG | |
| | GAAATCCCGACTGCAACTGGTTCAACAAGTGCCCCAGAGAAT | |
| | GCATCAGCGGCGTGTACACCGACGCCTATCCTCTGAGCCCCG | |
| | ACGCCTTTAACGTGGCCACCACAACACTGTACGCCAACACCA | |
| | GCAGAGTGAACCCCACCATCATGTACAGCTCCACCAGCAAGA | |
| | TCATCAACATGCTGAGACTGAAGAACGGCCAGCTGGAAGCCG | |
| | CCTACACCACCACAAGCTGCATCACCCACTTCGGCAAGGGCT | |
| | ACTGCTTCCACATCGTGGAAATCAACCAGACCTCTCTGAACA | |
| | CACTGCAGCCCATGCTGTTCAAGACCAGCATTCCCAAGATCT | |
| | GCAAAGTGACCAGCTGATGAGCTCGCTTTCTTGCTGTCCAAT | |
| | TTCTATTAAAGGTTCCTTTGTTCCCTAAGTCCAACTACTAAA | |
| | CTGGGGGATATTATGAAGGGCCTTGAGCATCTGGATTCTGCC | |
| | TAATAAAAAACATTTATTTTCATTGCAAaaaaaaaaaaaaaa | |
| | aaaaaaaaaaaaaaaaaaaaaaaaaaaaaaaaaaaaaaaaaa | |
| | aaaaaaaaaaaaaaaaaaaaaaaa | |
| PIV1084_5UTR_582/ | AGGTGTCAGAGTTTAACTTGAAGACTATTTCTAGGGATAATA | 772 |
| 3UTR_2 80A | TCCCTGCCACCATGGCCGAGAAGGGCAAGACCATCAGCAGCT | |
| PIV1 HN | ACTGGTCCACCACCAGAAACGACAACAGCACCGTGAACACCC | |
| del57-84 | ACATCAACACCCCAGCCGGCAGAACCTACATCTGGCTGCTGA | |
| PIV1084 | TCGCCACCACCATGCACGCTGCCCTGAGCCTGATCATCATGA | |
| DNA | TCCTGTGCATCGAGACAATCACCGAGCTGATCCGGCAAGAAG | |
| Underline = 5′ cap; | TGATCAGCCGGACCATCAACATCCAGTCCTCTGTGCAGAGCG | |
| bold = 5′UTR | GCATCCCCATCCTGCTGAACAAGCAGAGCAGAGATCTGACCC | |
| (5UTR_582) and | AGCTGATCGAGAAGTCCTGCAACAAGCAAGAGCTGGCCCAGA | |
| 3′ UTR (3UTR_2); | TCTGCGAGAACACAATCGCCATTCACCACGCCGACGGCATCA | |
| italics = KOZAK | CCCCTCTGGACCCTCACGATTTTTGGAGATGCCCTGTGGGCG | |
| sequence; | AGCCCCTGCTGAGCAACAACCCCAATATCAGCCTGCTGCCTG | |
| lowercase = | GACCTTCTCTGCTGAGCGGCAGCACAACAATCTCTGGCTGCG | |
| polyA tail | TCAGACTGCCCAGCCTGTCTATCGGAGATGCCATCTACGCCT | |
| (Amino acid | ACAGCAGCAACCTGATCACACAGGGCTGCGCCGACATCGGCA | |
| SEQ ID NO: 756) | AGAGCTATCAGGTTCTGCAGCTGGGCTACATCAGCCTGAACA | |
| | GCGACATGTACCCCGATCTGAACCCCGTGATCTCCCACACCT | |
| | ACGACATCAACGACAACCGCAAGTCCTGCTCCGTGATCGCCG | |
| | CTGGCACAAGAGGATACCAGCTGTGTAGCCTGCCTACCGTGA | |
| | ACGAAACCACCGACTACAGCTCCGAGGGCATCGAGGACCTGG | |
| | TGTTCGACATCCTGGACCTGAAGGGAAAGACAAAGAGCCACC | |
| | GGTACAAGAACGAGGACATCACCTTCGATCACCCCTTCAGCG | |
| | CCATGTATCCTAGCGTCGGCAGCGGCATCAAGATCGAGAACA | |
| | CCCTGGTGTTTCTCGGCTACGGCGGCCTGACAACACCTCTGC | |
| | AGGGCAACACCAAATGCGTGATCAAGAGCTGCCCTAACGTGA | |
| | ACCAGTCCGTGTGCAACGACGCCCTGAAGATCACCTGGCTGA | |
| | AGAAACGGCAGGTCGTGAACGTGCTGATCCACATTAACAACT | |
| | ACCTGAGCGACAGGCCCAAGATCGTGGTGGAAACAATCCCCA | |
| | TCACACAGAACTACCTGGGCGCCGAAGGCAGACTGCTGAAGC | |
| | TGGGCAAGAAGATCTACATCTACACCAGAAGCAGCGGCTGGC | |
| | ACTCCAACCTGCAGATCGGCAGCATCGATATCAACAAGCCCA | |
| | TGACCATCAATTGGGCCCCTCACAAGGTGCTGAGCAGACCCG | |
| | GAAATCCCGACTGCAACTGGTTCAACAAGTGCCCCAGAGAAT | |
| | GCATCAGCGGCGTGTACACCGACGCCTATCCTCTGAGCCCCG | |
| | ACGCCTTTAACGTGGCCACCACAACACTGTACGCCAACACCA | |
| | GCAGAGTGAACCCCACCATCATGTACAGCTCCACCAGCAAGA | |
| | TCATCAACATGCTGAGACTGAAGAACGGCCAGCTGGAAGCCG | |
| | CCTACACCACCACAAGCTGCATCACCCACTTCGGCAAGGGCT | |
| | ACTGCTTCCACATCGTGGAAATCAACCAGACCTCTCTGAACA | |
| | CACTGCAGCCCATGCTGTTCAAGACCAGCATTCCCAAGATCT | |
| | GCAAAGTGACCAGCTGATGAGCTCGCTTTCTTGCTGTCCAAT | |
| | TTCTATTAAAGGTTCCTTTGTTCCCTAAGTCCAACTACTAAA | |
| | CTGGGGGATATTATGAAGGGCCTTGAGCATCTGGATTCTGCC | |
| | TAATAAAAAACATTTATTTTCATTGCAAaaaaaaaaaaaaaa | |
| | aaaaaaaaaaaaaaaaaaaaaaaaaaaaaaaaaaaaaaaaaa | |
| | aaaaaaaaaaaaaaaaaaaaaaaa | |
| PIV1085_5UTR_582/ | AGGTGTCAGAGTTTAACTTGAAGACTATTTCTAGGGATAATA | 773 |
| 3UTR_2 80A | TCCCTGCCACCATGGCCGAGAAGGGCAAGACCATCAGCAGCT | |
| PIV1 HN | ACTGGTCCACCACCAGAAACGACAACAGCACCGTGAACACCC | |
| del57-129 | ACATCAACACCCCAGCCGGCAGAACCTACATCTGGCTGCTGA | |
| PIV1085 | TCGCCACCACCATGCACGCTGCCCTGAGCCTGATCATCATGA | |
| DNA | TCCTGTGCATCAACAAGCAAGAGCTGGCCCAGATCTGCGAGA | |
| Underline = 5′ cap; | ACACAATCGCCATTCACCACGCCGACGGCATCACCCCTCTGG | |
| bold = 5′UTR | ACCCTCACGATTTTTGGAGATGCCCTGTGGGCGAGCCCCTGC | |
| (5UTR_582) and | TGAGCAACAACCCCAATATCAGCCTGCTGCCTGGACCTTCTC | |
| 3′ UTR (3UTR_2); | TGCTGAGCGGCAGCACAACAATCTCTGGCTGCGTCAGACTGC | |
| italics = KOZAK | CCAGCCTGTCTATCGGAGATGCCATCTACGCCTACAGCAGCA | |
| sequence; | ACCTGATCACACAGGGCTGCGCCGACATCGGCAAGAGCTATC | |
| lowercase = | AGGTTCTGCAGCTGGGCTACATCAGCCTGAACAGCGACATGT | |
| polyA tail | ACCCCGATCTGAACCCCGTGATCTCCCACACCTACGACATCA | |
| (Amino acid | ACGACAACCGCAAGTCCTGCTCCGTGATCGCCGCTGGCACAA | |
| SEQ ID NO: 757) | GAGGATACCAGCTGTGTAGCCTGCCTACCGTGAACGAAACCA | |
| | CCGACTACAGCTCCGAGGGCATCGAGGACCTGGTGTTCGACA | |
| | TCCTGGACCTGAAGGGAAAGACAAAGAGCCACCGGTACAAGA | |
| | ACGAGGACATCACCTTCGATCACCCCTTCAGCGCCATGTATC | |
| | CTAGCGTCGGCAGCGGCATCAAGATCGAGAACACCCTGGTGT | |
| | TTCTCGGCTACGGCGGCCTGACAACACCTCTGCAGGGCAACA | |
| | CCAAATGCGTGATCAAGAGCTGCCCTAACGTGAACCAGTCCG | |
| | TGTGCAACGACGCCCTGAAGATCACCTGGCTGAAGAAACGGC | |
| | AGGTCGTGAACGTGCTGATCCACATTAACAACTACCTGAGCG | |
| | ACAGGCCCAAGATCGTGGTGGAAACAATCCCCATCACACAGA | |
| | ACTACCTGGGCGCCGAAGGCAGACTGCTGAAGCTGGGCAAGA | |
| | AGATCTACATCTACACCAGAAGCAGCGGCTGGCACTCCAACC | |
| | TGCAGATCGGCAGCATCGATATCAACAAGCCCATGACCATCA | |
| | ATTGGGCCCCTCACAAGGTGCTGAGCAGACCCGGAAATCCCG | |
| | ACTGCAACTGGTTCAACAAGTGCCCCAGAGAATGCATCAGCG | |
| | GCGTGTACACCGACGCCTATCCTCTGAGCCCCGACGCCTTTA | |
| | ACGTGGCCACCACAACACTGTACGCCAACACCAGCAGAGTGA | |
| | ACCCCACCATCATGTACAGCTCCACCAGCAAGATCATCAACA | |
| | TGCTGAGACTGAAGAACGGCCAGCTGGAAGCCGCCTACACCA | |
| | CCACAAGCTGCATCACCCACTTCGGCAAGGGCTACTGCTTCC | |
| | ACATCGTGGAAATCAACCAGACCTCTCTGAACACACTGCAGC | |
| | CCATGCTGTTCAAGACCAGCATTCCCAAGATCTGCAAAGTGA | |
| | CCAGCTGATGAGCTCGCTTTCTTGCTGTCCAATTTCTATTAA | |
| | AGGTTCCTTTGTTCCCTAAGTCCAACTACTAAACTGGGGGAT | |
| | ATTATGAAGGGCCTTGAGCATCTGGATTCTGCCTAATAAAAA | |
| | ACATTTATTTTCATTGCAAaaaaaaaaaaaaaaaaaaaaaaa | |
| | aaaaaaaaaaaaaaaaaaaaaaaaaaaaaaaaaaaaaaaaaa | |
| | aaaaaaaaaaaaaaa | |

TABLE 12
PIV1 HN RNA

| | SEQ ID |
|---|---|
| ID | NO: |
| PIV1 WT HN PIV1083 | 761 |
| UGA stop codon; (Amino acid SEQ ID NO: 750) | |
| PIV1 HN PIV1084 | 762 |
| UGA stop codon; (Amino acid SEQ ID NO: 756) | |
| PIV1 HN PIV1085 | 763 |
| UGA stop codon; (Amino acid SEQ ID NO: 757) | |

| | | SEQ ID |
|---|---|---|
| ID | Sequence | NO: |
| PIV1083_5UTR_579/ | AGGAGGACUGGGCGAACCUGCAUAGUGAUCAUAAGGUCAUCA | 774 |
| 3UTR_2 80A_modRNA | UAGCCACCAUGGCCGAGAAGGGCAAGACCAUCAGCAGCUACU | |
| PIV1 HN WT | GGUCCACCACCAGAAACGACAACAGCACCGUGAACACCCACA | |
| PIV1083 | UCAACACCCCAGCCGGCAGAACCUACAUCUGGCUGCUGAUCG | |
| Underline = 5′cap; | CCACCACCAUGCACGCUGCCCUGAGCCUGAUCAUCAUGAUCC | |
| bold = 5′UTR | UGUGCAUCGACCUCAUCAUCAAGCAGGAUACCUGCAUGAAGA | |
| (5UTR_579) and | CCAACAUCAUGACCGUGUCCAGCGUGAACGAGAGCGCCAAGA | |
| 3′UTR (3UTR_2); | CAAUCAAAGAGACAAUCACCGAGCUGAUCCGGCAAGAAGUGA | |
| italics = KOZAK | UCAGCCGGACCAUCAACAUCCAGUCCUCUGUGCAGAGCGGCA | |
| sequence; | UCCCCAUCCUGCUGAACAAGCAGAGCAGAGAUCUGACCCAGC | |
| lowercase = | UGAUCGAGAAGUCCUGCAACAAGCAAGAGCUGGCCCAGAUCU | |
| polyA tail | GCGAGAACACAAUCGCCAUUCACCACGCCGACGGCAUCACCC | |
| (Amino acid | CUCUGGACCCUCACGAUUUUUGGAGAUGCCCUGUGGGCGAGC | |
| SEQ ID NO: 750) | CCCUGCUGAGCAACAACCCCAAUAUCAGCCUGCUGCCUGGAC | |
| | CUUCUCUGCUGAGCGGCAGCACAACAAUCUCUGGCUGCGUCA | |
| | GACUGCCCAGCCUGUCUAUCGGAGAUGCCAUCUACGCCUACA | |
| | GCAGCAACCUGAUCACACAGGGCUGCGCCGACAUCGGCAAGA | |
| | GCUAUCAGGUUCUGCAGCUGGGCUACAUCAGCCUGAACAGCG | |
| | ACAUGUACCCCGAUCUGAACCCCGUGAUCUCCCACACCUACG | |
| | ACAUCAACGACAACCGCAAGUCCUGCUCCGUGAUCGCCGCUG | |
| | GCACAAGAGGAUACCAGCUGUGUAGCCUGCCUACCGUGAACG | |
| | AAACCACCGACUACAGCUCCGAGGGCAUCGAGGACCUGGUGU | |
| | UCGACAUCCUGGACCUGAAGGGAAAGACAAAGAGCCACCGGU | |
| | ACAAGAACGAGGACAUCACCUUCGAUCACCCCUUCAGCGCCA | |
| | UGUAUCCUAGCGUCGGCAGCGGCAUCAAGAUCGAGAACACCC | |
| | UGGUGUUUCUCGGCUACGGCGGCCUGACAACACCUCUGCAGG | |
| | GCAACACCAAAUGCGUGAUCAAGAGCUGCCCUAACGUGAACC | |
| | AGUCCGUGUGCAACGACGCCCUGAAGAUCACCUGGCUGAAGA | |
| | AACGGCAGGUCGUGAACGUGCUGAUCCACAUUAACAACUACC | |
| | UGAGCGACAGGCCCAAGAUCGUGGUGGAAACAAUCCCCAUCA | |
| | CACAGAACUACCUGGGCGCCGAAGGCAGACUGCUGAAGCUGG | |
| | GCAAGAAGAUCUACAUCUACACCAGAAGCAGCGGCUGGCACU | |
| | CCAACCUGCAGAUCGGCAGCAUCGAUAUCAACAAGCCCAUGA | |
| | CCAUCAAUUGGGCCCCUCACAAGGUGCUGAGCAGACCCGGAA | |
| | AUCCCGACUGCAACUGGUUCAACAAGUGCCCCAGAGAAUGCA | |
| | UCAGCGGCGUGUACACCGACGCCUAUCCUCUGAGCCCCGACG | |
| | CCUUUAACGUGGCCACCACAACACUGUACGCCAACACCAGCA | |
| | GAGUGAACCCCACCAUCAUGUACAGCUCCACCAGCAAGAUCA | |
| | UCAACAUGCUGAGACUGAAGAACGGCCAGCUGGAAGCCGCCU | |
| | ACACCACCACAAGCUGCAUCACCCACUUCGGCAAGGGCUACU | |
| | GCUUCCACAUCGUGGAAAUCAACCAGACCUCUCUGAACACAC | |
| | UGCAGCCCAUGCUGUUCAAGACCAGCAUUCCCAAGAUCUGCA | |
| | AAGUGACCAGCUGAUGAGCUCGCUUUCUUGCUGUCCAAUUUC | |
| | UAUUAAAGGUUCCUUUGUUCCCUAAGUCCAACUACUAAACUG | |
| | GGGGAUAUUAUGAAGGGCCUUGAGCAUCUGGAUUCUGCCUAA | |
| | UAAAAAACAUUUAUUUUCAUUGCAAaaaaaaaaaaaaaaaaa | |
| | aaaaaaaaaaaaaaaaaaaaaaaaaaaaaaaaaaaaaaaaaa | |
| | aaaaaaaaaaaaaaaaaaaaa | |
| PIV1084_5UTR_579/ | AGGAGGACUGGGCGAACCUGCAUAGUGAUCAUAAGGUCAUCA | 775 |
| 3UTR_2 80A_modRNA | UAGCCACCAUGGCCGAGAAGGGCAAGACCAUCAGCAGCUACU | |
| PIV1 HN | GGUCCACCACCAGAAACGACAACAGCACCGUGAACACCCACA | |
| del57-84 | UCAACACCCCAGCCGGCAGAACCUACAUCUGGCUGCUGAUCG | |
| PIV1084 | CCACCACCAUGCACGCUGCCCUGAGCCUGAUCAUCAUGAUCC | |
| Underline = 5′cap; | UGUGCAUCGAGACAAUCACCGAGCUGAUCCGGCAAGAAGUGA | |
| bold = 5′UTR | UCAGCCGGACCAUCAACAUCCAGUCCUCUGUGCAGAGCGGCA | |
| (5UTR_579) and | UCCCCAUCCUGCUGAACAAGCAGAGCAGAGAUCUGACCCAGC | |
| 3′UTR (3UTR_2); | UGAUCGAGAAGUCCUGCAACAAGCAAGAGCUGGCCCAGAUCU | |
| italics = KOZAK | GCGAGAACACAAUCGCCAUUCACCACGCCGACGGCAUCACCC | |
| sequence; | CUCUGGACCCUCACGAUUUUUGGAGAUGCCCUGUGGGCGAGC | |
| lowercase = | CCCUGCUGAGCAACAACCCCAAUAUCAGCCUGCUGCCUGGAC | |
| polyA tail | CUUCUCUGCUGAGCGGCAGCACAACAAUCUCUGGCUGCGUCA | |
| (Amino acid | GACUGCCCAGCCUGUCUAUCGGAGAUGCCAUCUACGCCUACA | |
| SEQ ID NO: 756) | GCAGCAACCUGAUCACACAGGGCUGCGCCGACAUCGGCAAGA | |
| | GCUAUCAGGUUCUGCAGCUGGGCUACAUCAGCCUGAACAGCG | |
| | ACAUGUACCCCGAUCUGAACCCCGUGAUCUCCCACACCUACG | |
| | ACAUCAACGACAACCGCAAGUCCUGCUCCGUGAUCGCCGCUG | |
| | GCACAAGAGGAUACCAGCUGUGUAGCCUGCCUACCGUGAACG | |
| | AAACCACCGACUACAGCUCCGAGGGCAUCGAGGACCUGGUGU | |
| | UCGACAUCCUGGACCUGAAGGGAAAGACAAAGAGCCACCGGU | |
| | ACAAGAACGAGGACAUCACCUUCGAUCACCCCUUCAGCGCCA | |
| | UGUAUCCUAGCGUCGGCAGCGGCAUCAAGAUCGAGAACACCC | |
| | UGGUGUUUCUCGGCUACGGCGGCCUGACAACACCUCUGCAGG | |
| | GCAACACCAAAUGCGUGAUCAAGAGCUGCCCUAACGUGAACC | |
| | AGUCCGUGUGCAACGACGCCCUGAAGAUCACCUGGCUGAAGA | |
| | AACGGCAGGUCGUGAACGUGCUGAUCCACAUUAACAACUACC | |
| | UGAGCGACAGGCCCAAGAUCGUGGUGGAAACAAUCCCCAUCA | |
| | CACAGAACUACCUGGGCGCCGAAGGCAGACUGCUGAAGCUGG | |
| | GCAAGAAGAUCUACAUCUACACCAGAAGCAGCGGCUGGCACU | |
| | CCAACCUGCAGAUCGGCAGCAUCGAUAUCAACAAGCCCAUGA | |
| | CCAUCAAUUGGGCCCCUCACAAGGUGCUGAGCAGACCCGGAA | |
| | AUCCCGACUGCAACUGGUUCAACAAGUGCCCCAGAGAAUGCA | |
| | UCAGCGGCGUGUACACCGACGCCUAUCCUCUGAGCCCCGACG | |
| | CCUUUAACGUGGCCACCACAACACUGUACGCCAACACCAGCA | |
| | GAGUGAACCCCACCAUCAUGUACAGCUCCACCAGCAAGAUCA | |
| | UCAACAUGCUGAGACUGAAGAACGGCCAGCUGGAAGCCGCCU | |
| | ACACCACCACAAGCUGCAUCACCCACUUCGGCAAGGGCUACU | |
| | GCUUCCACAUCGUGGAAAUCAACCAGACCUCUCUGAACACAC | |
| | UGCAGCCCAUGCUGUUCAAGACCAGCAUUCCCAAGAUCUGCA | |
| | AAGUGACCAGCUGAUGAGCUCGCUUUCUUGCUGUCCAAUUUC | |
| | UAUUAAAGGUUCCUUUGUUCCCUAAGUCCAACUACUAAACUG | |
| | GGGGAUAUUAUGAAGGGCCUUGAGCAUCUGGAUUCUGCCUAA | |
| | UAAAAAACAUUUAUUUUCAUUGCAAaaaaaaaaaaaaaaaaa | |
| | aaaaaaaaaaaaaaaaaaaaaaaaaaaaaaaaaaaaaaaaaa | |
| | aaaaaaaaaaaaaaaaaaaaa | |
| PIV1085_5UTR_579/ | AGGAGGACUGGGCGAACCUGCAUAGUGAUCAUAAGGUCAUCA | 776 |
| 3UTR_2 80A_modRNA | UAGCCACCAUGGCCGAGAAGGGCAAGACCAUCAGCAGCUACU | |
| PIV1 HN | GGUCCACCACCAGAAACGACAACAGCACCGUGAACACCCACA | |
| del57-129 | UCAACACCCCAGCCGGCAGAACCUACAUCUGGCUGCUGAUCG | |
| PIV1085 | CCACCACCAUGCACGCUGCCCUGAGCCUGAUCAUCAUGAUCC | |
| Underline = 5′cap; | UGUGCAUCAACAAGCAAGAGCUGGCCCAGAUCUGCGAGAACA | |
| bold = 5′UTR | CAAUCGCCAUUCACCACGCCGACGGCAUCACCCCUCUGGACC | |
| (5UTR_579) and | CUCACGAUUUUUGGAGAUGCCCUGUGGGCGAGCCCCUGCUGA | |
| 3′UTR (3UTR_2); | GCAACAACCCCAAUAUCAGCCUGCUGCCUGGACCUUCUCUGC | |
| italics = KOZAK | UGAGCGGCAGCACAACAAUCUCUGGCUGCGUCAGACUGCCCA | |
| sequence; | GCCUGUCUAUCGGAGAUGCCAUCUACGCCUACAGCAGCAACC | |
| lowercase = | UGAUCACACAGGGCUGCGCCGACAUCGGCAAGAGCUAUCAGG | |
| polyA tail | UUCUGCAGCUGGGCUACAUCAGCCUGAACAGCGACAUGUACC | |
| (Amino acid | CCGAUCUGAACCCCGUGAUCUCCCACACCUACGACAUCAACG | |
| SEQ ID NO: 757) | ACAACCGCAAGUCCUGCUCCGUGAUCGCCGCUGGCACAAGAG | |
| | GAUACCAGCUGUGUAGCCUGCCUACCGUGAACGAAACCACCG | |
| | ACUACAGCUCCGAGGGCAUCGAGGACCUGGUGUUCGACAUCC | |
| | UGGACCUGAAGGGAAAGACAAAGAGCCACCGGUACAAGAACG | |
| | AGGACAUCACCUUCGAUCACCCCUUCAGCGCCAUGUAUCCUA | |
| | GCGUCGGCAGCGGCAUCAAGAUCGAGAACACCCUGGUGUUUC | |
| | UCGGCUACGGCGGCCUGACAACACCUCUGCAGGGCAACACCA | |
| | AAUGCGUGAUCAAGAGCUGCCCUAACGUGAACCAGUCCGUGU | |
| | GCAACGACGCCCUGAAGAUCACCUGGCUGAAGAAACGGCAGG | |
| | UCGUGAACGUGCUGAUCCACAUUAACAACUACCUGAGCGACA | |
| | GGCCCAAGAUCGUGGUGGAAACAAUCCCCAUCACACAGAACU | |
| | ACCUGGGCGCCGAAGGCAGACUGCUGAAGCUGGGCAAGAAGA | |
| | UCUACAUCUACACCAGAAGCAGCGGCUGGCACUCCAACCUGC | |
| | AGAUCGGCAGCAUCGAUAUCAACAAGCCCAUGACCAUCAAUU | |
| | GGGCCCCUCACAAGGUGCUGAGCAGACCCGGAAAUCCCGACU | |
| | GCAACUGGUUCAACAAGUGCCCCAGAGAAUGCAUCAGCGGCG | |
| | UGUACACCGACGCCUAUCCUCUGAGCCCCGACGCCUUUAACG | |
| | UGGCCACCACAACACUGUACGCCAACACCAGCAGAGUGAACC | |
| | CCACCAUCAUGUACAGCUCCACCAGCAAGAUCAUCAACAUGC | |
| | UGAGACUGAAGAACGGCCAGCUGGAAGCCGCCUACACCACCA | |
| | CAAGCUGCAUCACCCACUUCGGCAAGGGCUACUGCUUCCACA | |
| | UCGUGGAAAUCAACCAGACCUCUCUGAACACACUGCAGCCCA | |
| | UGCUGUUCAAGACCAGCAUUCCCAAGAUCUGCAAAGUGACCA | |
| | GCUGAUGAGCUCGCUUUCUUGCUGUCCAAUUUCUAUUAAAGG | |
| | UUCCUUUGUUCCCUAAGUCCAACUACUAAACUGGGGGAUAUU | |
| | AUGAAGGGCCUUGAGCAUCUGGAUUCUGCCUAAUAAAAAACA | |
| | UUUAUUUUCAUUGCAAaaaaaaaaaaaaaaaaaaaaaaaaaa | |
| | aaaaaaaaaaaaaaaaaaaaaaaaaaaaaaaaaaaaaaaaaa | |
| | aaaaaaaaaaaa | |
| PIV1083_5UTR_582/ | AGGUGUCAGAGUUUAACUUGAAGACUAUUUCUAGGGAUAAUA | 777 |
| 3UTR_2 80A_modRNA | UCCCUGCCACCAUGGCCGAGAAGGGCAAGACCAUCAGCAGCU | |
| PIV1 HN WT | ACUGGUCCACCACCAGAAACGACAACAGCACCGUGAACACCC | |
| PIV1083 | ACAUCAACACCCCAGCCGGCAGAACCUACAUCUGGCUGCUGA | |
| Underline = 5′cap; | UCGCCACCACCAUGCACGCUGCCCUGAGCCUGAUCAUCAUGA | |
| bold = 5′UTR | UCCUGUGCAUCGACCUCAUCAUCAAGCAGGAUACCUGCAUGA | |
| (5UTR_582) and | AGACCAACAUCAUGACCGUGUCCAGCGUGAACGAGAGCGCCA | |
| 3′UTR (3UTR_2); | AGACAAUCAAAGAGACAAUCACCGAGCUGAUCCGGCAAGAAG | |
| italics = KOZAK | UGAUCAGCCGGACCAUCAACAUCCAGUCCUCUGUGCAGAGCG | |
| sequence; | GCAUCCCCAUCCUGCUGAACAAGCAGAGCAGAGAUCUGACCC | |
| lowercase = | AGCUGAUCGAGAAGUCCUGCAACAAGCAAGAGCUGGCCCAGA | |
| polyA tail | UCUGCGAGAACACAAUCGCCAUUCACCACGCCGACGGCAUCA | |
| (Amino acid | CCCCUCUGGACCCUCACGAUUUUUGGAGAUGCCCUGUGGGCG | |
| SEQ ID NO: 750) | AGCCCCUGCUGAGCAACAACCCCAAUAUCAGCCUGCUGCCUG | |
| | GACCUUCUCUGCUGAGCGGCAGCACAACAAUCUCUGGCUGCG | |
| | UCAGACUGCCCAGCCUGUCUAUCGGAGAUGCCAUCUACGCCU | |
| | ACAGCAGCAACCUGAUCACACAGGGCUGCGCCGACAUCGGCA | |
| | AGAGCUAUCAGGUUCUGCAGCUGGGCUACAUCAGCCUGAACA | |
| | GCGACAUGUACCCCGAUCUGAACCCCGUGAUCUCCCACACCU | |
| | ACGACAUCAACGACAACCGCAAGUCCUGCUCCGUGAUCGCCG | |
| | CUGGCACAAGAGGAUACCAGCUGUGUAGCCUGCCUACCGUGA | |
| | ACGAAACCACCGACUACAGCUCCGAGGGCAUCGAGGACCUGG | |
| | UGUUCGACAUCCUGGACCUGAAGGGAAAGACAAAGAGCCACC | |
| | GGUACAAGAACGAGGACAUCACCUUCGAUCACCCCUUCAGCG | |
| | CCAUGUAUCCUAGCGUCGGCAGCGGCAUCAAGAUCGAGAACA | |
| | CCCUGGUGUUUCUCGGCUACGGCGGCCUGACAACACCUCUGC | |
| | AGGGCAACACCAAAUGCGUGAUCAAGAGCUGCCCUAACGUGA | |
| | ACCAGUCCGUGUGCAACGACGCCCUGAAGAUCACCUGGCUGA | |
| | AGAAACGGCAGGUCGUGAACGUGCUGAUCCACAUUAACAACU | |
| | ACCUGAGCGACAGGCCCAAGAUCGUGGUGGAAACAAUCCCCA | |
| | UCACACAGAACUACCUGGGCGCCGAAGGCAGACUGCUGAAGC | |
| | UGGGCAAGAAGAUCUACAUCUACACCAGAAGCAGCGGCUGGC | |
| | ACUCCAACCUGCAGAUCGGCAGCAUCGAUAUCAACAAGCCCA | |
| | UGACCAUCAAUUGGGCCCCUCACAAGGUGCUGAGCAGACCCG | |
| | GAAAUCCCGACUGCAACUGGUUCAACAAGUGCCCCAGAGAAU | |
| | GCAUCAGCGGCGUGUACACCGACGCCUAUCCUCUGAGCCCCG | |
| | ACGCCUUUAACGUGGCCACCACAACACUGUACGCCAACACCA | |
| | GCAGAGUGAACCCCACCAUCAUGUACAGCUCCACCAGCAAGA | |
| | UCAUCAACAUGCUGAGACUGAAGAACGGCCAGCUGGAAGCCG | |
| | CCUACACCACCACAAGCUGCAUCACCCACUUCGGCAAGGGCU | |
| | ACUGCUUCCACAUCGUGGAAAUCAACCAGACCUCUCUGAACA | |
| | CACUGCAGCCCAUGCUGUUCAAGACCAGCAUUCCCAAGAUCU | |
| | GCAAAGUGACCAGCUGAUGAGCUCGCUUUCUUGCUGUCCAAU | |
| | UUCUAUUAAAGGUUCCUUUGUUCCCUAAGUCCAACUACUAAA | |
| | CUGGGGGAUAUUAUGAAGGGCCUUGAGCAUCUGGAUUCUGCC | |
| | UAAUAAAAAACAUUUAUUUUCAUUGCAAaaaaaaaaaaaaaa | |
| | aaaaaaaaaaaaaaaaaaaaaaaaaaaaaaaaaaaaaaaaaa | |
| | aaaaaaaaaaaaaaaaaaaaaaaa | |
| PIV1084_5UTR_582/ | AGGUGUCAGAGUUUAACUUGAAGACUAUUUCUAGGGAUAAUA | 778 |
| 3UTR_2 80A_modRNA | UCCCUGCCACCAUGGCCGAGAAGGGCAAGACCAUCAGCAGCU | |
| PIV1 HN | ACUGGUCCACCACCAGAAACGACAACAGCACCGUGAACACCC | |
| del57-84 | ACAUCAACACCCCAGCCGGCAGAACCUACAUCUGGCUGCUGA | |
| PIV1084 | UCGCCACCACCAUGCACGCUGCCCUGAGCCUGAUCAUCAUGA | |
| Underline = 5′cap; | UCCUGUGCAUCGAGACAAUCACCGAGCUGAUCCGGCAAGAAG | |
| bold = 5′UTR | UGAUCAGCCGGACCAUCAACAUCCAGUCCUCUGUGCAGAGCG | |
| (5UTR_582) and | GCAUCCCCAUCCUGCUGAACAAGCAGAGCAGAGAUCUGACCC | |
| 3′UTR (3UTR_2); | AGCUGAUCGAGAAGUCCUGCAACAAGCAAGAGCUGGCCCAGA | |
| italics = KOZAK | UCUGCGAGAACACAAUCGCCAUUCACCACGCCGACGGCAUCA | |
| sequence; | CCCCUCUGGACCCUCACGAUUUUUGGAGAUGCCCUGUGGGCG | |
| lowercase = | AGCCCCUGCUGAGCAACAACCCCAAUAUCAGCCUGCUGCCUG | |
| polyA tail | GACCUUCUCUGCUGAGCGGCAGCACAACAAUCUCUGGCUGCG | |
| (Amino acid | UCAGACUGCCCAGCCUGUCUAUCGGAGAUGCCAUCUACGCCU | |
| SEQ ID NO: 756) | ACAGCAGCAACCUGAUCACACAGGGCUGCGCCGACAUCGGCA | |
| | AGAGCUAUCAGGUUCUGCAGCUGGGCUACAUCAGCCUGAACA | |
| | GCGACAUGUACCCCGAUCUGAACCCCGUGAUCUCCCACACCU | |
| | ACGACAUCAACGACAACCGCAAGUCCUGCUCCGUGAUCGCCG | |
| | CUGGCACAAGAGGAUACCAGCUGUGUAGCCUGCCUACCGUGA | |
| | ACGAAACCACCGACUACAGCUCCGAGGGCAUCGAGGACCUGG | |
| | UGUUCGACAUCCUGGACCUGAAGGGAAAGACAAAGAGCCACC | |
| | GGUACAAGAACGAGGACAUCACCUUCGAUCACCCCUUCAGCG | |
| | CCAUGUAUCCUAGCGUCGGCAGCGGCAUCAAGAUCGAGAACA | |
| | CCCUGGUGUUUCUCGGCUACGGCGGCCUGACAACACCUCUGC | |
| | AGGGCAACACCAAAUGCGUGAUCAAGAGCUGCCCUAACGUGA | |
| | ACCAGUCCGUGUGCAACGACGCCCUGAAGAUCACCUGGCUGA | |
| | AGAAACGGCAGGUCGUGAACGUGCUGAUCCACAUUAACAACU | |
| | ACCUGAGCGACAGGCCCAAGAUCGUGGUGGAAACAAUCCCCA | |
| | UCACACAGAACUACCUGGGCGCCGAAGGCAGACUGCUGAAGC | |
| | UGGGCAAGAAGAUCUACAUCUACACCAGAAGCAGCGGCUGGC | |
| | ACUCCAACCUGCAGAUCGGCAGCAUCGAUAUCAACAAGCCCA | |
| | UGACCAUCAAUUGGGCCCCUCACAAGGUGCUGAGCAGACCCG | |
| | GAAAUCCCGACUGCAACUGGUUCAACAAGUGCCCCAGAGAAU | |
| | GCAUCAGCGGCGUGUACACCGACGCCUAUCCUCUGAGCCCCG | |
| | ACGCCUUUAACGUGGCCACCACAACACUGUACGCCAACACCA | |
| | GCAGAGUGAACCCCACCAUCAUGUACAGCUCCACCAGCAAGA | |
| | UCAUCAACAUGCUGAGACUGAAGAACGGCCAGCUGGAAGCCG | |
| | CCUACACCACCACAAGCUGCAUCACCCACUUCGGCAAGGGCU | |
| | ACUGCUUCCACAUCGUGGAAAUCAACCAGACCUCUCUGAACA | |
| | CACUGCAGCCCAUGCUGUUCAAGACCAGCAUUCCCAAGAUCU | |
| | GCAAAGUGACCAGCUGAUGAGCUCGCUUUCUUGCUGUCCAAU | |
| | UUCUAUUAAAGGUUCCUUUGUUCCCUAAGUCCAACUACUAAA | |
| | CUGGGGGAUAUUAUGAAGGGCCUUGAGCAUCUGGAUUCUGCC | |
| | UAAUAAAAAACAUUUAUUUUCAUUGCAAaaaaaaaaaaaaaa | |
| | aaaaaaaaaaaaaaaaaaaaaaaaaaaaaaaaaaaaaaaaaa | |
| | aaaaaaaaaaaaaaaaaaaaaaaa | |
| PIV1085_5UTR_582/ | AGGUGUCAGAGUUUAACUUGAAGACUAUUUCUAGGGAUAAUA | 779 |
| 3UTR_2 80A_modRNA | UCCCUGCCACCAUGGCCGAGAAGGGCAAGACCAUCAGCAGCU | |
| PIV1 HN | ACUGGUCCACCACCAGAAACGACAACAGCACCGUGAACACCC | |
| del57-129 | ACAUCAACACCCCAGCCGGCAGAACCUACAUCUGGCUGCUGA | |
| PIV1085 | UCGCCACCACCAUGCACGCUGCCCUGAGCCUGAUCAUCAUGA | |
| Underline = 5′cap; | UCCUGUGCAUCAACAAGCAAGAGCUGGCCCAGAUCUGCGAGA | |
| bold = 5′UTR | ACACAAUCGCCAUUCACCACGCCGACGGCAUCACCCCUCUGG | |
| (5UTR_582) and | ACCCUCACGAUUUUUGGAGAUGCCCUGUGGGCGAGCCCCUGC | |
| 3′UTR (3UTR_2); | UGAGCAACAACCCCAAUAUCAGCCUGCUGCCUGGACCUUCUC | |
| italics = KOZAK | UGCUGAGCGGCAGCACAACAAUCUCUGGCUGCGUCAGACUGC | |
| sequence; | CCAGCCUGUCUAUCGGAGAUGCCAUCUACGCCUACAGCAGCA | |
| lowercase = | ACCUGAUCACACAGGGCUGCGCCGACAUCGGCAAGAGCUAUC | |
| polyA tail | AGGUUCUGCAGCUGGGCUACAUCAGCCUGAACAGCGACAUGU | |
| (Amino acid | ACCCCGAUCUGAACCCCGUGAUCUCCCACACCUACGACAUCA | |
| SEQ ID NO: 757) | ACGACAACCGCAAGUCCUGCUCCGUGAUCGCCGCUGGCACAA | |
| | GAGGAUACCAGCUGUGUAGCCUGCCUACCGUGAACGAAACCA | |
| | CCGACUACAGCUCCGAGGGCAUCGAGGACCUGGUGUUCGACA | |
| | UCCUGGACCUGAAGGGAAAGACAAAGAGCCACCGGUACAAGA | |
| | ACGAGGACAUCACCUUCGAUCACCCCUUCAGCGCCAUGUAUC | |
| | CUAGCGUCGGCAGCGGCAUCAAGAUCGAGAACACCCUGGUGU | |
| | UUCUCGGCUACGGCGGCCUGACAACACCUCUGCAGGGCAACA | |
| | CCAAAUGCGUGAUCAAGAGCUGCCCUAACGUGAACCAGUCCG | |
| | UGUGCAACGACGCCCUGAAGAUCACCUGGCUGAAGAAACGGC | |
| | AGGUCGUGAACGUGCUGAUCCACAUUAACAACUACCUGAGCG | |
| | ACAGGCCCAAGAUCGUGGUGGAAACAAUCCCCAUCACACAGA | |
| | ACUACCUGGGCGCCGAAGGCAGACUGCUGAAGCUGGGCAAGA | |
| | AGAUCUACAUCUACACCAGAAGCAGCGGCUGGCACUCCAACC | |
| | UGCAGAUCGGCAGCAUCGAUAUCAACAAGCCCAUGACCAUCA | |
| | AUUGGGCCCCUCACAAGGUGCUGAGCAGACCCGGAAAUCCCG | |
| | ACUGCAACUGGUUCAACAAGUGCCCCAGAGAAUGCAUCAGCG | |
| | GCGUGUACACCGACGCCUAUCCUCUGAGCCCCGACGCCUUUA | |
| | ACGUGGCCACCACAACACUGUACGCCAACACCAGCAGAGUGA | |
| | ACCCCACCAUCAUGUACAGCUCCACCAGCAAGAUCAUCAACA | |
| | UGCUGAGACUGAAGAACGGCCAGCUGGAAGCCGCCUACACCA | |
| | CCACAAGCUGCAUCACCCACUUCGGCAAGGGCUACUGCUUCC | |
| | ACAUCGUGGAAAUCAACCAGACCUCUCUGAACACACUGCAGC | |
| | CCAUGCUGUUCAAGACCAGCAUUCCCAAGAUCUGCAAAGUGA | |
| | CCAGCUGAUGAGCUCGCUUUCUUGCUGUCCAAUUUCUAUUAA | |
| | AGGUUCCUUUGUUCCCUAAGUCCAACUACUAAACUGGGGGAU | |
| | AUUAUGAAGGGCCUUGAGCAUCUGGAUUCUGCCUAAUAAAAA | |
| | ACAUUUAUUUUCAUUGCAAaaaaaaaaaaaaaaaaaaaaaaa | |
| | aaaaaaaaaaaaaaaaaaaaaaaaaaaaaaaaaaaaaaaaaa | |
| | aaaaaaaaaaaaaaa | |

### D. PIV3 Sequences

TABLE 13
PIV3 F Polypeptides

| ID | SEQ ID NO: |
|---|---|
| Full Length F0 of PIV3 (GenBank GI: AGT75285.1) | 690 |
| PIV3 F PIV3135 | 691 |
| PIV3 F PIV3140 | 692 |
| PIV3 F PIV3135 Ecto-Foldon | 693 |
| PIV3 F PIV3140 Ecto-Foldon | 694 |
| PIV3 F PIV3008 | 712 |

TABLE 14
PIV3 F DNA

| | | SEQ ID |
|---|---|---|
| ID | | NO: |
| PIV3 WT F | | 695 |
| TGA stop codon; (Amino acid SEQ ID NO: 690) | | |
| PIV3 F PIV3135 | | 696 |
| TGA stop codon; (Amino acid SEQ ID NO: 691) | | |
| PIV3 F PIV3140 | | 697 |
| TGA stop codon; (Amino acid SEQ ID NO: 692) | | |
| PIV3 F PIV3135 Ecto-Foldon | | 702 |
| TGA stop codon; (Amino acid SEQ ID NO: 693) | | |
| PIV3 F PIV3140 Ecto-Foldon | | 703 |
| TGA stop codon; (Amino acid SEQ ID NO: 694) | | |
| PIV3 F PIV3008 | | 713 |
| TGA stop codon; (Amino acid SEQ ID NO: 712) | | |
| | | SEQ ID |
| ID | Sequence | NO: |
| PIV3135_5UTR_ | AGGAGGACTGGGCGAACCTGCATAGTGATCATAAGGTCATCA | 698 |
| 579/3UTR_ | TAGCCACCATGCTGATCTCCATCCTGCTGATCATCACCACAAT | |
| 2 80A | GATCATGGCCAGCCACTGCCAGATCGACATCACCAAGCTGCAG | |
| PIV3 F | CACGTGGGCGTGCTGGTCAATAGCCCTAAGGGCATGAAGATC | |
| PIV3135 | AGCCAGAACTTCGAGACACGCTACCTGATCCTGTCTCTGATCC | |
| DNA | CCAAGATCGAGGACAGCAACAGCTGCGGCGACCAGCAGATCA | |
| Underline = | AGCAGTACAAGCGGCTGCTGGACAGACTGATCATCCCTCTGTA | |
| 5′ cap; bold = | CGACGGCCTGCGGCTGCAGAAAGATGTGATCGTGACCAATCA | |
| 5′UTR | AGAGAGCAACGAGAACACAGACCCCAGAACCGAGAGATTCTTC | |
| (5UTR_563) | GGCGGCGTGATCGGCACAATCGCCCTGGGAGTTGCTACAAGC | |
| and 3′ UTR | GCCCAGATTACAGCCGCCGTGGCTCTGGTGGAAGCCAAGCAG | |
| (3UTR_2); | GCCAGAAGCGACATCGAGAAGCTGAAAGAGGCCATCCGGGAC | |
| italics = | ACCAACAAGGCCGTGCAGTCTGTGCAAAGCAGCGTGGGCAAT | |
| KOZAK | CTGATCGTGGCCATTAAGAGCGTGCAGGACTACGTGAACAAAG | |
| sequence; | AGATCGTCCCCTCTATCGCCAGACTGGGATGTGAAGCTGCCG | |
| lowercase = | GACTGCAGCTGGGAATTGCCCTGACACAGCACTACAGCTGCCT | |
| polyA tail | GACCAACATCTTCGGCGACAACATCGGCAGCCTGCAAGAGAA | |
| (Amino acid | GGGCATTAAGCTGCAGGGAATCGCCAGCTGCTACCGCACCAA | |
| SEQ ID NO: | CATCACCGAGATCTTCACCACCAGCACCGTGGATAAGTACGAC | |
| 691) | ATCTACGACCTGCTGTTCACCGAGAGCATCAAAGTGCGCGTGA | |
| | TCGACGTGGACCTGAACGACTACAGCATCACCCTGCAAGTGCG | |
| | GCTGCCTCTGCTGACCAGACTGCTGAACACCCAGATCTACAAG | |
| | GTGGACAGCATCTCCTACAACATCCAGAACCGCGAGTGGTACA | |
| | TCCCACTGCCTAGCCACATTATGACCAAGGGCGCCTTTCTCGG | |
| | CGGAGCCGATGTGAAAGAGTGCATCGAGGCCTTCAGCAGCTA | |
| | CATCTGCCCTAGCGATCCTGGCTTCGTGCTGAACCACGAGATG | |
| | GAAAGCTGCCTGAGCGGCAACATCTCTCAGTGCCCTAGAACCA | |
| | CCGTGACCTCCGACATCGTGCCCAGATACGCCTTTGTGAATGG | |
| | CGGCGTGGTGGCCAACTGCATCACCACCACCTGTACCTGCAA | |
| | CGGCATCGGCAACCGGATCAACCAGCCTCCAGATCAGGGCGT | |
| | GAAGATTATCACCCACAAAGAGTGTAACACCATCGGCATCAAC | |
| | GGCATGCTGTTCAATACCAACAAAGAGGGCACCCTGGCCTTCT | |
| | ACACCCCTGACGATATCACCCTGAACAACAGCGTGGCCCTGGA | |
| | TCCTATCGACATCTCCATCGAGCTGAACAAGGCCAAGAGCGAC | |
| | CTGGAAGAAAGCAAAGAGTGGATCCGGCGGAGCAACCAGAAG | |
| | CTGGATAGCATCGGAAGCTGGCACCAGAGCAGCACCACCATC | |
| | ATCGTGATCCTGATTATGATGATTATCCTGTTCATCATCAACATT | |
| | ACCATCATCACGATCGCCATCAAGTACTACCGGATCCAGAAAC | |
| | GGAACCGCGTGGACCAGAATGACAAGCCCTACGTGCTGACAA | |
| | ACAAGTGATGAGCTCGCTTTCTTGCTGTCCAATTTCTATTAAAG | |
| | GTTCCTTTGTTCCCTAAGTCCAACTACTAAACTGGGGGATATT | |
| | ATGAAGGGCCTTGAGCATCTGGATTCTGCCTAATAAAAAACA | |
| | TTTATTTTCATTGCAAaaaaaaaaaaaaaaaaaaaaaaaaaaaaaaaaaaa | |
| | aaaaaaaaaaaaaaaaaaaaaaaaaaaaaaaaaaaaaaaaaaaaa | |
| PIV3140_5UTR_ | AGGAGGACTGGGCGAACCTGCATAGTGATCATAAGGTCATCA | 699 |
| 579/3UTR_ | TAGCCACCATGCTGATCTCCATCCTGCTGATCATCACCACAAT | |
| 2 80A | GATCATGGCCAGCCACTGCCAGATCGACATCACCAAGCTGCAG | |
| PIV3 F | CACGTGGGCGTGCTGGTCAATAGCCCTAAGGGCATGAAGATC | |
| PIV3140 | AGCCAGAACTTCGAGACACGCTACCTGATCCTGTCTCTGATCC | |
| Underline = | CCAAGATCGAGGACAGCAACAGCTGCGGCGACCAGCAGATCA | |
| 5′ cap; bold = | AGCAGTACAAGCGGCTGCTGGACAGACTGATCATCCCTCTGTA | |
| 5′UTR | CGACGGCCTGCGGCTGCAGAAAGATGTGATCGTGACCAATCA | |
| (5UTR_563) | AGAGAGCAACGAGAACACAGACCCCAGAACCGAGAGATTCTTC | |
| and 3′ UTR | GGCGGCGTGATCGGCACAATCGCCCTGGGAGTTGCTACAAGC | |
| (3UTR_2); | GCCCAGATTACAGCCGCCGTGGCTCTGGTGGAAGCCAAGCAG | |
| italics = | GCCAGAAGCGACATCGAGAAGCTGAAAGAGGCCATCCGGGAC | |
| KOZAK | ACCAACAAGGCCGTGCAGTGCGTGCAAAGCAGCGTGGGCAAT | |
| sequence; | CTGATCTGCGCCATTAAGAGCGTGCAGGACTACGTGAACAAAG | |
| lowercase = | AGATCGTCCCCTCTATCGCCAGACTGGGATGTGAAGCTGCCG | |
| polyA tail | GACTGCAGCTGGGAATTGCCCTGACACAGCACTACAGCTGCCT | |
| (Amino acid | GACCAACATCTTCGGCGACAACATCGGCAGCCTGCAAGAGAA | |
| SEQ ID NO: | GGGCATTAAGCTGCAGGGAATCGCCAGCTGCTACCGCACCAA | |
| 692) | CATCACCGAGATCTTCACCACCAGCACCGTGGATAAGTACGAC | |
| | ATCTACGACCTGCTGTTCACCGAGAGCATCAAAGTGCGCGTGA | |
| | TCGACGTGGACCTGAACGACTACAGCATCACCCTGCAAGTGCG | |
| | GCTGCCTCTGCTGACCAGACTGCTGAACACCCAGATCTACAAG | |
| | GTGGACAGCATCTCCTACAACATCCAGAACCGCGAGTGGTACA | |
| | TCCCACTGCCTAGCCACATTATGACCAAGGGCGCCTTTCTCGG | |
| | CGGAGCCGATGTGAAAGAGTGCATCGAGGCCTTCAGCAGCTA | |
| | CATCTGCCCTAGCGATCCTGGCTTCGTGCTGAACCACGAGATG | |
| | GAAAGCTGCCTGAGCGGCAACATCTCTCAGTGCCCTAGAACCA | |
| | CCGTGACCTCCGACATCGTGCCCAGATACGCCTTTGTGAATGG | |
| | CGGCGTGGTGGCCAACTGCATCACCACCACCTGTACCTGCAA | |
| | CGGCATCGGCAACCGGATCAACCAGCCTCCAGATCAGGGCGT | |
| | GAAGATTATCACCCACAAAGAGTGTAACACCATCGGCATCAAC | |
| | GGCATGCTGTTCAATACCAACAAAGAGGGCACCCTGGCCTTCT | |
| | ACACCCCTGACGATATCACCCTGAACAACAGCGTGGCCCTGGA | |
| | TCCTATCGACATCTCCATCGAGCTGAACAAGCTCAAGAGCGAC | |
| | CTGGAAGAACTGAAAGAGTGGATCCGGCGGAGCAACCAGAAG | |
| | CTGGATAGCATCGGAAGCTGGCACCAGAGCAGCACCACCATC | |
| | ATCGTGATCCTGATTATGATGATTATCCTGTTCATCATCAACATT | |
| | ACCATCATCACGATCGCCATCAAGTACTACCGGATCCAGAAAC | |
| | GGAACCGCGTGGACCAGAATGACAAGCCCTACGTGCTGACAA | |
| | ACAAGTGATGAGCTCGCTTTCTTGCTGTCCAATTTCTATTAAAG | |
| | GTTCCTTTGTTCCCTAAGTCCAACTACTAAACTGGGGGATATT | |
| | ATGAAGGGCCTTGAGCATCTGGATTCTGCCTAATAAAAAACA | |
| | TTTATTTTCATTGCAAaaaaaaaaaaaaaaaaaaaaaaaaaaaaaaaaaaa | |
| | aaaaaaaaaaaaaaaaaaaaaaaaaaaaaaaaaaaaaaaaaaaaa | |
| PIV3135_5UTR_ | AGGTGTCAGAGTTTAACTTGAAGACTATTTCTAGGGATAATAT | 700 |
| 582/3UTR_ | CCCTGCCACCATGCTGATCTCCATCCTGCTGATCATCACCACA | |
| 2 80A | ATGATCATGGCCAGCCACTGCCAGATCGACATCACCAAGCTGC | |
| PIV3 F | AGCACGTGGGCGTGCTGGTCAATAGCCCTAAGGGCATGAAGA | |
| PIV3135 | TCAGCCAGAACTTCGAGACACGCTACCTGATCCTGTCTCTGAT | |
| DNA | CCCCAAGATCGAGGACAGCAACAGCTGCGGCGACCAGCAGAT | |
| Underline = | CAAGCAGTACAAGCGGCTGCTGGACAGACTGATCATCCCTCTG | |
| 5′ cap; bold = | TACGACGGCCTGCGGCTGCAGAAAGATGTGATCGTGACCAATC | |
| 5′UTR | AAGAGAGCAACGAGAACACAGACCCCAGAACCGAGAGATTCTT | |
| (5UTR_582) | CGGCGGCGTGATCGGCACAATCGCCCTGGGAGTTGCTACAAG | |
| and 3′ UTR | CGCCCAGATTACAGCCGCCGTGGCTCTGGTGGAAGCCAAGCA | |
| (3UTR_2); | GGCCAGAAGCGACATCGAGAAGCTGAAAGAGGCCATCCGGGA | |
| italics = | CACCAACAAGGCCGTGCAGTCTGTGCAAAGCAGCGTGGGCAA | |
| KOZAK | TCTGATCGTGGCCATTAAGAGCGTGCAGGACTACGTGAACAAA | |
| sequence; | GAGATCGTCCCCTCTATCGCCAGACTGGGATGTGAAGCTGCC | |
| lowercase = | GGACTGCAGCTGGGAATTGCCCTGACACAGCACTACAGCTGC | |
| polyA tail | CTGACCAACATCTTCGGCGACAACATCGGCAGCCTGCAAGAGA | |
| (Amino acid | AGGGCATTAAGCTGCAGGGAATCGCCAGCTGCTACCGCACCA | |
| SEQ ID NO: | ACATCACCGAGATCTTCACCACCAGCACCGTGGATAAGTACGA | |
| 691) | CATCTACGACCTGCTGTTCACCGAGAGCATCAAAGTGCGCGTG | |
| | ATCGACGTGGACCTGAACGACTACAGCATCACCCTGCAAGTGC | |
| | GGCTGCCTCTGCTGACCAGACTGCTGAACACCCAGATCTACAA | |
| | GGTGGACAGCATCTCCTACAACATCCAGAACCGCGAGTGGTAC | |
| | ATCCCACTGCCTAGCCACATTATGACCAAGGGCGCCTTTCTCG | |
| | GCGGAGCCGATGTGAAAGAGTGCATCGAGGCCTTCAGCAGCT | |
| | ACATCTGCCCTAGCGATCCTGGCTTCGTGCTGAACCACGAGAT | |
| | GGAAAGCTGCCTGAGCGGCAACATCTCTCAGTGCCCTAGAACC | |
| | ACCGTGACCTCCGACATCGTGCCCAGATACGCCTTTGTGAATG | |
| | GCGGCGTGGTGGCCAACTGCATCACCACCACCTGTACCTGCA | |
| | ACGGCATCGGCAACCGGATCAACCAGCCTCCAGATCAGGGCG | |
| | TGAAGATTATCACCCACAAAGAGTGTAACACCATCGGCATCAA | |
| | CGGCATGCTGTTCAATACCAACAAAGAGGGCACCCTGGCCTTC | |
| | TACACCCCTGACGATATCACCCTGAACAACAGCGTGGCCCTGG | |
| | ATCCTATCGACATCTCCATCGAGCTGAACAAGGCCAAGAGCGA | |
| | CCTGGAAGAAAGCAAAGAGTGGATCCGGCGGAGCAACCAGAA | |
| | GCTGGATAGCATCGGAAGCTGGCACCAGAGCAGCACCACCAT | |
| | CATCGTGATCCTGATTATGATGATTATCCTGTTCATCATCAACAT | |
| | TACCATCATCACGATCGCCATCAAGTACTACCGGATCCAGAAA | |
| | CGGAACCGCGTGGACCAGAATGACAAGCCCTACGTGCTGACA | |
| | AACAAGTGATGAGCTCGCTTTCTTGCTGTCCAATTTCTATTAAA | |
| | GGTTCCTTTGTTCCCTAAGTCCAACTACTAAACTGGGGGATAT | |
| | TATGAAGGGCCTTGAGCATCTGGATTCTGCCTAATAAAAAAC | |
| | ATTTATTTTCATTGCAAaaaaaaaaaaaaaaaaaaaaaaaaaaaaaaaaaa | |
| | aaaaaaaaaaaaaaaaaaaaaaaaaaaaaaaaaaaaaaaaaaaaaa | |
| PIV3140_5UTR_ | AGGTGTCAGAGTTTAACTTGAAGACTATTTCTAGGGATAATAT | |
| 582/3UTR_ | CCCTGCCACCATGCTGATCTCCATCCTGCTGATCATCACCACA | |
| 2 80A | ATGATCATGGCCAGCCACTGCCAGATCGACATCACCAAGCTGC | 701 |
| PIV3 F | AGCACGTGGGCGTGCTGGTCAATAGCCCTAAGGGCATGAAGA | |
| PIV3140 | TCAGCCAGAACTTCGAGACACGCTACCTGATCCTGTCTCTGAT | |
| DNA | CCCCAAGATCGAGGACAGCAACAGCTGCGGCGACCAGCAGAT | |
| Underline = | CAAGCAGTACAAGCGGCTGCTGGACAGACTGATCATCCCTCTG | |
| 5′ cap; bold = | TACGACGGCCTGCGGCTGCAGAAAGATGTGATCGTGACCAATC | |
| 5′UTR | AAGAGAGCAACGAGAACACAGACCCCAGAACCGAGAGATTCTT | |
| (5UTR_582) | CGGCGGCGTGATCGGCACAATCGCCCTGGGAGTTGCTACAAG | |
| and 3′ UTR | CGCCCAGATTACAGCCGCCGTGGCTCTGGTGGAAGCCAAGCA | |
| (3UTR_2); | GGCCAGAAGCGACATCGAGAAGCTGAAAGAGGCCATCCGGGA | |
| italics = | CACCAACAAGGCCGTGCAGTGCGTGCAAAGCAGCGTGGGCAA | |
| KOZAK | TCTGATCTGCGCCATTAAGAGCGTGCAGGACTACGTGAACAAA | |
| sequence; | GAGATCGTCCCCTCTATCGCCAGACTGGGATGTGAAGCTGCC | |
| lowercase = | GGACTGCAGCTGGGAATTGCCCTGACACAGCACTACAGCTGC | |
| polyA tail | CTGACCAACATCTTCGGCGACAACATCGGCAGCCTGCAAGAGA | |
| (Amino acid | AGGGCATTAAGCTGCAGGGAATCGCCAGCTGCTACCGCACCA | |
| SEQ ID NO: | ACATCACCGAGATCTTCACCACCAGCACCGTGGATAAGTACGA | |
| 692) | CATCTACGACCTGCTGTTCACCGAGAGCATCAAAGTGCGCGTG | |
| | ATCGACGTGGACCTGAACGACTACAGCATCACCCTGCAAGTGC | |
| | GGCTGCCTCTGCTGACCAGACTGCTGAACACCCAGATCTACAA | |
| | GGTGGACAGCATCTCCTACAACATCCAGAACCGCGAGTGGTAC | |
| | ATCCCACTGCCTAGCCACATTATGACCAAGGGCGCCTTTCTCG | |
| | GCGGAGCCGATGTGAAAGAGTGCATCGAGGCCTTCAGCAGCT | |
| | ACATCTGCCCTAGCGATCCTGGCTTCGTGCTGAACCACGAGAT | |
| | GGAAAGCTGCCTGAGCGGCAACATCTCTCAGTGCCCTAGAACC | |
| | ACCGTGACCTCCGACATCGTGCCCAGATACGCCTTTGTGAATG | |
| | GCGGCGTGGTGGCCAACTGCATCACCACCACCTGTACCTGCA | |
| | ACGGCATCGGCAACCGGATCAACCAGCCTCCAGATCAGGGCG | |
| | TGAAGATTATCACCCACAAAGAGTGTAACACCATCGGCATCAA | |
| | CGGCATGCTGTTCAATACCAACAAAGAGGGCACCCTGGCCTTC | |
| | TACACCCCTGACGATATCACCCTGAACAACAGCGTGGCCCTGG | |
| | ATCCTATCGACATCTCCATCGAGCTGAACAAGCTCAAGAGCGA | |
| | CCTGGAAGAACTGAAAGAGTGGATCCGGCGGAGCAACCAGAA | |
| | GCTGGATAGCATCGGAAGCTGGCACCAGAGCAGCACCACCAT | |
| | CATCGTGATCCTGATTATGATGATTATCCTGTTCATCATCAACAT | |
| | TACCATCATCACGATCGCCATCAAGTACTACCGGATCCAGAAA | |
| | CGGAACCGCGTGGACCAGAATGACAAGCCCTACGTGCTGACA | |
| | AACAAGTGATGAGCTCGCTTTCTTGCTGTCCAATTTCTATTAAA | |
| | GGTTCCTTTGTTCCCTAAGTCCAACTACTAAACTGGGGGATAT | |
| | TATGAAGGGCCTTGAGCATCTGGATTCTGCCTAATAAAAAAC | |
| | ATTTATTTTCATTGCAAaaaaaaaaaaaaaaaaaaaaaaaaaaaaaaaaaa | |
| | aaaaaaaaaaaaaaaaaaaaaaaaaaaaaaaaaaaaaaaaaaaaaa | |

TABLE 15
PIV3 F RNA

| | | SEQ ID |
|---|---|---|
| ID | | NO: |
| PIV3 WT F | | 766 |
| UGA stop codon; (Amino acid SEQ ID NO: 690) | | |
| PIV3 F PIV3135 | | 704 |
| UGA stop codon; (Amino acid SEQ ID NO: 691) | | |
| PIV3 F PIV3140 | | 705 |
| UGA stop codon; (Amino acid SEQ ID NO: 692) | | |
| PIV3 F PIV3135 Ecto-Foldon | | 710 |
| UGA stop codon; (Amino acid SEQ ID NO: 693) | | |
| PIV3 F PIV3140 Ecto-Foldon | | 711 |
| UGA stop codon; (Amino acid SEQ ID NO: 694) | | |
| PIV3 F PIV3008 | | 714 |
| UGA stop codon; (Amino acid SEQ ID NO: 712) | | |
| | | SEQ ID |
| ID | Sequence | NO: |
| PIV3135_5UTR_ | AGGAGGACUGGGCGAACCUGCAUAGUGAUCAUAAGGUCAUC | 706 |
| 579/3UT | AUAGCCACCAUGCUGAUCUCCAUCCUGCUGAUCAUCACCACA | |
| 2 80A_R_ | AUGAUCAUGGCCAGCCACUGCCAGAUCGACAUCACCAAGCUG | |
| modRNA | CAGCACGUGGGCGUGCUGGUCAAUAGCCCUAAGGGCAUGAA | |
| Underline = | GAUCAGCCAGAACUUCGAGACACGCUACCUGAUCCUGUCUCU | |
| 5′ cap; | GAUCCCCAAGAUCGAGGACAGCAACAGCUGCGGCGACCAGCA | |
| bold = 5′UTR | GAUCAAGCAGUACAAGCGGCUGCUGGACAGACUGAUCAUCCC | |
| (5UTR_579) | UCUGUACGACGGCCUGCGGCUGCAGAAAGAUGUGAUCGUGA | |
| and 3′ UTR | CCAAUCAAGAGAGCAACGAGAACACAGACCCCAGAACCGAGA | |
| (3UTR_2); | GAUUCUUCGGCGGCGUGAUCGGCACAAUCGCCCUGGGAGUU | |
| italics = | GCUACAAGCGCCCAGAUUACAGCCGCCGUGGCUCUGGUGGA | |
| KOZAK | AGCCAAGCAGGCCAGAAGCGACAUCGAGAAGCUGAAAGAGGC | |
| sequence; | CAUCCGGGACACCAACAAGGCCGUGCAGUCUGUGCAAAGCA | |
| lowercase = | GCGUGGGCAAUCUGAUCGUGGCCAUUAAGAGCGUGCAGGAC | |
| polyA tail | UACGUGAACAAAGAGAUCGUCCCCUCUAUCGCCAGACUGGGA | |
| (Amino acid | UGUGAAGCUGCCGGACUGCAGCUGGGAAUUGCCCUGACACA | |
| SEQ ID NO: | GCACUACAGCUGCCUGACCAACAUCUUCGGCGACAACAUCGG | |
| 691) | CAGCCUGCAAGAGAAGGGCAUUAAGCUGCAGGGAAUCGCCA | |
| | GCUGCUACCGCACCAACAUCACCGAGAUCUUCACCACCAGCA | |
| | CCGUGGAUAAGUACGACAUCUACGACCUGCUGUUCACCGAGA | |
| | GCAUCAAAGUGCGCGUGAUCGACGUGGACCUGAACGACUAC | |
| | AGCAUCACCCUGCAAGUGCGGCUGCCUCUGCUGACCAGACU | |
| | GCUGAACACCCAGAUCUACAAGGUGGACAGCAUCUCCUACAA | |
| | CAUCCAGAACCGCGAGUGGUACAUCCCACUGCCUAGCCACAU | |
| | UAUGACCAAGGGCGCCUUUCUCGGCGGAGCCGAUGUGAAAG | |
| | AGUGCAUCGAGGCCUUCAGCAGCUACAUCUGCCCUAGCGAU | |
| | CCUGGCUUCGUGCUGAACCACGAGAUGGAAAGCUGCCUGAG | |
| | CGGCAACAUCUCUCAGUGCCCUAGAACCACCGUGACCUCCGA | |
| | CAUCGUGCCCAGAUACGCCUUUGUGAAUGGCGGCGUGGUGG | |
| | CCAACUGCAUCACCACCACCUGUACCUGCAACGGCAUCGGCA | |
| | ACCGGAUCAACCAGCCUCCAGAUCAGGGCGUGAAGAUUAUCA | |
| | CCCACAAAGAGUGUAACACCAUCGGCAUCAACGGCAUGCUGU | |
| | UCAAUACCAACAAAGAGGGCACCCUGGCCUUCUACACCCCUG | |
| | ACGAUAUCACCCUGAACAACAGCGUGGCCCUGGAUCCUAUCG | |
| | ACAUCUCCAUCGAGCUGAACAAGGCCAAGAGCGACCUGGAAG | |
| | AAAGCAAAGAGUGGAUCCGGCGGAGCAACCAGAAGCUGGAUA | |
| | GCAUCGGAAGCUGGCACCAGAGCAGCACCACCAUCAUCGUGA | |
| | UCCUGAUUAUGAUGAUUAUCCUGUUCAUCAUCAACAUUACCA | |
| | UCAUCACGAUCGCCAUCAAGUACUACCGGAUCCAGAAACGGA | |
| | ACCGCGUGGACCAGAAUGACAAGCCCUACGUGCUGACAAACA | |
| | AGUGAUGAGCUCGCUUUCUUGCUGUCCAAUUUCUAUUAAAG | |
| | GUUCCUUUGUUCCCUAAGUCCAACUACUAAACUGGGGGAUA | |
| | UUAUGAAGGGCCUUGAGCAUCUGGAUUCUGCCUAAUAAAAA | |
| | ACAUUUAUUUUCAUUGCAAaaaaaaaaaaaaaaaaaaaaaaaaaaaaa | |
| | aaaaaaaaaaaaaaaaaaaaaaaaaaaaaaaaaaaaaaaaaaaaaaaaaaa | |
| PIV3140_5UTR_ | AGGAGGACUGGGCGAACCUGCAUAGUGAUCAUAAGGUCAUC | 707 |
| 579/3UTR_ | AUAGCCACCAUGCUGAUCUCCAUCCUGCUGAUCAUCACCACA | |
| 2 80A_ | AUGAUCAUGGCCAGCCACUGCCAGAUCGACAUCACCAAGCUG | |
| modRNA | CAGCACGUGGGCGUGCUGGUCAAUAGCCCUAAGGGCAUGAA | |
| Underline = | GAUCAGCCAGAACUUCGAGACACGCUACCUGAUCCUGUCUCU | |
| 5′ cap; bold | GAUCCCCAAGAUCGAGGACAGCAACAGCUGCGGCGACCAGCA | |
| = 5′ UTR | GAUCAAGCAGUACAAGCGGCUGCUGGACAGACUGAUCAUCCC | |
| (5UTR_579) | UCUGUACGACGGCCUGCGGCUGCAGAAAGAUGUGAUCGUGA | |
| and 3′ UTR | CCAAUCAAGAGAGCAACGAGAACACAGACCCCAGAACCGAGA | |
| (3UTR_2); | GAUUCUUCGGCGGCGUGAUCGGCACAAUCGCCCUGGGAGUU | |
| italics = | GCUACAAGCGCCCAGAUUACAGCCGCCGUGGCUCUGGUGGA | |
| KOZAK | AGCCAAGCAGGCCAGAAGCGACAUCGAGAAGCUGAAAGAGGC | |
| sequence; | CAUCCGGGACACCAACAAGGCCGUGCAGUGCGUGCAAAGCA | |
| lowercase = | GCGUGGGCAAUCUGAUCUGCGCCAUUAAGAGCGUGCAGGAC | |
| play tail | UACGUGAACAAAGAGAUCGUCCCCUCUAUCGCCAGACUGGGA | |
| (Amino acid | UGUGAAGCUGCCGGACUGCAGCUGGGAAUUGCCCUGACACA | |
| SEQ ID NO: | GCACUACAGCUGCCUGACCAACAUCUUCGGCGACAACAUCGG | |
| 692) | CAGCCUGCAAGAGAAGGGCAUUAAGCUGCAGGGAAUCGCCA | |
| | GCUGCUACCGCACCAACAUCACCGAGAUCUUCACCACCAGCA | |
| | CCGUGGAUAAGUACGACAUCUACGACCUGCUGUUCACCGAGA | |
| | GCAUCAAAGUGCGCGUGAUCGACGUGGACCUGAACGACUAC | |
| | AGCAUCACCCUGCAAGUGCGGCUGCCUCUGCUGACCAGACU | |
| | GCUGAACACCCAGAUCUACAAGGUGGACAGCAUCUCCUACAA | |
| | CAUCCAGAACCGCGAGUGGUACAUCCCACUGCCUAGCCACAU | |
| | UAUGACCAAGGGCGCCUUUCUCGGCGGAGCCGAUGUGAAAG | |
| | AGUGCAUCGAGGCCUUCAGCAGCUACAUCUGCCCUAGCGAU | |
| | CCUGGCUUCGUGCUGAACCACGAGAUGGAAAGCUGCCUGAG | |
| | CGGCAACAUCUCUCAGUGCCCUAGAACCACCGUGACCUCCGA | |
| | CAUCGUGCCCAGAUACGCCUUUGUGAAUGGCGGCGUGGUGG | |
| | CCAACUGCAUCACCACCACCUGUACCUGCAACGGCAUCGGCA | |
| | ACCGGAUCAACCAGCCUCCAGAUCAGGGCGUGAAGAUUAUCA | |
| | CCCACAAAGAGUGUAACACCAUCGGCAUCAACGGCAUGCUGU | |
| | UCAAUACCAACAAAGAGGGCACCCUGGCCUUCUACACCCCUG | |
| | ACGAUAUCACCCUGAACAACAGCGUGGCCCUGGAUCCUAUCG | |
| | ACAUCUCCAUCGAGCUGAACAAGCUCAAGAGCGACCUGGAAG | |
| | AACUGAAAGAGUGGAUCCGGCGGAGCAACCAGAAGCUGGAUA | |
| | GCAUCGGAAGCUGGCACCAGAGCAGCACCACCAUCAUCGUGA | |
| | UCCUGAUUAUGAUGAUUAUCCUGUUCAUCAUCAACAUUACCA | |
| | UCAUCACGAUCGCCAUCAAGUACUACCGGAUCCAGAAACGGA | |
| | ACCGCGUGGACCAGAAUGACAAGCCCUACGUGCUGACAAACA | |
| | AGUGAUGAGCUCGCUUUCUUGCUGUCCAAUUUCUAUUAAAG | |
| | GUUCCUUUGUUCCCUAAGUCCAACUACUAAACUGGGGGAUA | |
| | UUAUGAAGGGCCUUGAGCAUCUGGAUUCUGCCUAAUAAAAA | |
| | ACAUUUAUUUUCAUUGCAAaaaaaaaaaaaaaaaaaaaaaaaaaaaaa | |
| | aaaaaaaaaaaaaaaaaaaaaaaaaaaaaaaaaaaaaaaaaaaaaaaaaaa | |
| PIV3135_5UTR_ | AGGUGUCAGAGUUUAACUUGAAGACUAUUUCUAGGGAUAAU | 708 |
| 582/3UTR_ | AUCCCUGCCACCAUGCUGAUCUCCAUCCUGCUGAUCAUCACC | |
| 2 80A_ | ACAAUGAUCAUGGCCAGCCACUGCCAGAUCGACAUCACCAAG | |
| modRNA | CUGCAGCACGUGGGCGUGCUGGUCAAUAGCCCUAAGGGCAU | |
| Underline = | GAAGAUCAGCCAGAACUUCGAGACACGCUACCUGAUCCUGUC | |
| 5′ cap; | UCUGAUCCCCAAGAUCGAGGACAGCAACAGCUGCGGCGACCA | |
| bold = 5′UTR | GCAGAUCAAGCAGUACAAGCGGCUGCUGGACAGACUGAUCAU | |
| (5UTR_582) | CCCUCUGUACGACGGCCUGCGGCUGCAGAAAGAUGUGAUCG | |
| and 3′ UTR | UGACCAAUCAAGAGAGCAACGAGAACACAGACCCCAGAACCG | |
| (3UTR_2); | AGAGAUUCUUCGGCGGCGUGAUCGGCACAAUCGCCCUGGGA | |
| italics = | GUUGCUACAAGCGCCCAGAUUACAGCCGCCGUGGCUCUGGU | |
| KOZAK | GGAAGCCAAGCAGGCCAGAAGCGACAUCGAGAAGCUGAAAGA | |
| sequence; | GGCCAUCCGGGACACCAACAAGGCCGUGCAGUCUGUGCAAA | |
| lowercase = | GCAGCGUGGGCAAUCUGAUCGUGGCCAUUAAGAGCGUGCAG | |
| polyA tail | GACUACGUGAACAAAGAGAUCGUCCCCUCUAUCGCCAGACUG | |
| (Amino acid | GGAUGUGAAGCUGCCGGACUGCAGCUGGGAAUUGCCCUGAC | |
| SEQ ID NO: | ACAGCACUACAGCUGCCUGACCAACAUCUUCGGCGACAACAU | |
| 691) | CGGCAGCCUGCAAGAGAAGGGCAUUAAGCUGCAGGGAAUCG | |
| | CCAGCUGCUACCGCACCAACAUCACCGAGAUCUUCACCACCA | |
| | GCACCGUGGAUAAGUACGACAUCUACGACCUGCUGUUCACC | |
| | GAGAGCAUCAAAGUGCGCGUGAUCGACGUGGACCUGAACGA | |
| | CUACAGCAUCACCCUGCAAGUGCGGCUGCCUCUGCUGACCA | |
| | GACUGCUGAACACCCAGAUCUACAAGGUGGACAGCAUCUCCU | |
| | ACAACAUCCAGAACCGCGAGUGGUACAUCCCACUGCCUAGCC | |
| | ACAUUAUGACCAAGGGCGCCUUUCUCGGCGGAGCCGAUGUG | |
| | AAAGAGUGCAUCGAGGCCUUCAGCAGCUACAUCUGCCCUAGC | |
| | GAUCCUGGCUUCGUGCUGAACCACGAGAUGGAAAGCUGCCU | |
| | GAGCGGCAACAUCUCUCAGUGCCCUAGAACCACCGUGACCUC | |
| | CGACAUCGUGCCCAGAUACGCCUUUGUGAAUGGCGGCGUGG | |
| | UGGCCAACUGCAUCACCACCACCUGUACCUGCAACGGCAUCG | |
| | GCAACCGGAUCAACCAGCCUCCAGAUCAGGGCGUGAAGAUUA | |
| | UCACCCACAAAGAGUGUAACACCAUCGGCAUCAACGGCAUGC | |
| | UGUUCAAUACCAACAAAGAGGGCACCCUGGCCUUCUACACCC | |
| | CUGACGAUAUCACCCUGAACAACAGCGUGGCCCUGGAUCCUA | |
| | UCGACAUCUCCAUCGAGCUGAACAAGGCCAAGAGCGACCUGG | |
| | AAGAAAGCAAAGAGUGGAUCCGGCGGAGCAACCAGAAGCUGG | |
| | AUAGCAUCGGAAGCUGGCACCAGAGCAGCACCACCAUCAUCG | |
| | UGAUCCUGAUUAUGAUGAUUAUCCUGUUCAUCAUCAACAUUA | |
| | CCAUCAUCACGAUCGCCAUCAAGUACUACCGGAUCCAGAAAC | |
| | GGAACCGCGUGGACCAGAAUGACAAGCCCUACGUGCUGACAA | |
| | ACAAGUGAUGAGCUCGCUUUCUUGCUGUCCAAUUUCUAUUA | |
| | AAGGUUCCUUUGUUCCCUAAGUCCAACUACUAAACUGGGGG | |
| | AUAUUAUGAAGGGCCUUGAGCAUCUGGAUUCUGCCUAAUAA | |
| | AAAACAUUUAUUUUCAUUGCAAaaaaaaaaaaaaaaaaaaaaaaaaa | |
| | aaaaaaaaaaaaaaaaaaaaaaaaaaaaaaaaaaaaaaaaaaaaaaaaaaaaaa | |
| | a | |
| PIV3140_5UTR_ | AGGUGUCAGAGUUUAACUUGAAGACUAUUUCUAGGGAUAAU | 709 |
| 582/3UTR_ | AUCCCUGCCACCAUGCUGAUCUCCAUCCUGCUGAUCAUCACC | |
| 2 80A_ | ACAAUGAUCAUGGCCAGCCACUGCCAGAUCGACAUCACCAAG | |
| modRNA | CUGCAGCACGUGGGCGUGCUGGUCAAUAGCCCUAAGGGCAU | |
| Underline = | GAAGAUCAGCCAGAACUUCGAGACACGCUACCUGAUCCUGUC | |
| 5′ cap; | UCUGAUCCCCAAGAUCGAGGACAGCAACAGCUGCGGCGACCA | |
| bold = 5′UTR | GCAGAUCAAGCAGUACAAGCGGCUGCUGGACAGACUGAUCAU | |
| (5UTR_582) | CCCUCUGUACGACGGCCUGCGGCUGCAGAAAGAUGUGAUCG | |
| and 3′ UTR | UGACCAAUCAAGAGAGCAACGAGAACACAGACCCCAGAACCG | |
| (3UTR_2); | AGAGAUUCUUCGGCGGCGUGAUCGGCACAAUCGCCCUGGGA | |
| italics = | GUUGCUACAAGCGCCCAGAUUACAGCCGCCGUGGCUCUGGU | |
| KOZAK | GGAAGCCAAGCAGGCCAGAAGCGACAUCGAGAAGCUGAAAGA | |
| sequence; | GGCCAUCCGGGACACCAACAAGGCCGUGCAGUGCGUGCAAA | |
| lowercase = | GCAGCGUGGGCAAUCUGAUCUGCGCCAUUAAGAGCGUGCAG | |
| polyA tail | GACUACGUGAACAAAGAGAUCGUCCCCUCUAUCGCCAGACUG | |
| (Amino acid | GGAUGUGAAGCUGCCGGACUGCAGCUGGGAAUUGCCCUGAC | |
| SEQ ID NO: | ACAGCACUACAGCUGCCUGACCAACAUCUUCGGCGACAACAU | |
| 692) | CGGCAGCCUGCAAGAGAAGGGCAUUAAGCUGCAGGGAAUCG | |
| | CCAGCUGCUACCGCACCAACAUCACCGAGAUCUUCACCACCA | |
| | GCACCGUGGAUAAGUACGACAUCUACGACCUGCUGUUCACC | |
| | GAGAGCAUCAAAGUGCGCGUGAUCGACGUGGACCUGAACGA | |
| | CUACAGCAUCACCCUGCAAGUGCGGCUGCCUCUGCUGACCA | |
| | GACUGCUGAACACCCAGAUCUACAAGGUGGACAGCAUCUCCU | |
| | ACAACAUCCAGAACCGCGAGUGGUACAUCCCACUGCCUAGCC | |
| | ACAUUAUGACCAAGGGCGCCUUUCUCGGCGGAGCCGAUGUG | |
| | AAAGAGUGCAUCGAGGCCUUCAGCAGCUACAUCUGCCCUAGC | |
| | GAUCCUGGCUUCGUGCUGAACCACGAGAUGGAAAGCUGCCU | |
| | GAGCGGCAACAUCUCUCAGUGCCCUAGAACCACCGUGACCUC | |
| | CGACAUCGUGCCCAGAUACGCCUUUGUGAAUGGCGGCGUGG | |
| | UGGCCAACUGCAUCACCACCACCUGUACCUGCAACGGCAUCG | |
| | GCAACCGGAUCAACCAGCCUCCAGAUCAGGGCGUGAAGAUUA | |
| | UCACCCACAAAGAGUGUAACACCAUCGGCAUCAACGGCAUGC | |
| | UGUUCAAUACCAACAAAGAGGGCACCCUGGCCUUCUACACCC | |
| | CUGACGAUAUCACCCUGAACAACAGCGUGGCCCUGGAUCCUA | |
| | UCGACAUCUCCAUCGAGCUGAACAAGCUCAAGAGCGACCUGG | |
| | AAGAACUGAAAGAGUGGAUCCGGCGGAGCAACCAGAAGCUG | |
| | GAUAGCAUCGGAAGCUGGCACCAGAGCAGCACCACCAUCAUC | |
| | GUGAUCCUGAUUAUGAUGAUUAUCCUGUUCAUCAUCAACAUU | |
| | ACCAUCAUCACGAUCGCCAUCAAGUACUACCGGAUCCAGAAA | |
| | CGGAACCGCGUGGACCAGAAUGACAAGCCCUACGUGCUGAC | |
| | AAACAAGUGAUGAGCUCGCUUUCUUGCUGUCCAAUUUCUAU | |
| | UAAAGGUUCCUUUGUUCCCUAAGUCCAACUACUAAACUGGG | |
| | GGAUAUUAUGAAGGGCCUUGAGCAUCUGGAUUCUGCCUAAU | |
| | AAAAAACAUUUAUUUUCAUUGCAAaaaaaaaaaaaaaaaaaaaaaaa | |
| | aaaaaaaaaaaaaaaaaaaaaaaaaaaaaaaaaaaaaaaaaaaaaaaaaaaaaa | |
| | aaa | |

TABLE 16
PIV3 HN Polypeptides

| ID | SEQ ID NO: |
|---|---|
| Full Length of PIV3 WT HN PIV3223 (GenBank GI: AGT75286.1) | 724 |
| PIV3 HN PIV3224 | 725 |
| PIV3 HN PIV3225 | 726 |
| Full Length of PIV3 WT HN (GenBank GI: AGT75294.1) | 745 |
| Full Length of PIV3 WT HN (GenBank GI: AGT75270.1) | 746 |
| Full Length of PIV3 WT HN (GenBank GI: AAA46849.1) | 747 |
| Full Length of PIV3 WT HN (GenBank GI: CAA81294.1) | 748 |
| Full Length of PIV3 WT HN (GenBank GI: ABZ85673.1) | 749 |

TABLE 17
PIV3 HN DNA

| | | SEQ ID |
|---|---|---|
| ID | | NO: |
| PIV3 WT HN PIV3223 | | 727 |
| TGA stop codon; (Amino acid SEQ ID NO: 724) | | |
| PIV3 HN PIV3224 | | 728 |
| TGA stop codon; (Amino acid SEQ ID NO: 725) | | |
| PIV3 HN PIV3225 | | 729 |
| TGA stop codon; (Amino acid SEQ ID NO: 726) | | |
| | | SEQ ID |
| ID | Sequence | NO: |
| PIV3223_5UTR_ | AGGAGGACTGGGCGAACCTGCATAGTGATCATAAGGTCATCA | 730 |
| 579/3UTR_ | TAGCCACCATGGAATACTGGAAGCACACCAACCACGGCAAGG | |
| 2 80A | ACGCCGGCAATGAGCTGGAAACAAGCACAGCTACCCACGGCA | |
| PIV3 HN WT | ACAAGCTGACCAACAAGATCACCTACATCCTGTGGACCATCAC | |
| PIV3223 | ACTGGTGCTGCTGAGCATCGTGTTCATCATCGTGCTGACCAAT | |
| DNA | AGCATCAAGAGCGAGAAGGCCAGAGAGAGCCTGCTGCAGGAC | |
| Underline = | ATCAACAACGAGTTCATGGAAGTGACCGAGAAGATCCAGGTGG | |
| 5′ cap; bold = | CCAGCGACAACACCAACGACCTGATTCAGAGCGGCGTGAACA | |
| 5′UTR | CCCGGCTGCTGACAATTCAGAGCCACGTGCAGAACTATATCCC | |
| (5UTR_579) | CATCAGCCTGACACAGCAGATCAGCGACCTGCGGAAGTTCATC | |
| and 3′ UTR | AGCGAGATCACCATCCGGAACGACAATCAAGAGGTGCCACCA | |
| (3UTR_2); | CAGCGGATCACCCACGATGTGGGAATCAAGCCTCTGAACCCC | |
| italics = | GACGACTTCTGGCGGTGTACATCTGGCCTGCCTAGCCTGATGA | |
| KOZAK | AGACCCCTAAGATCCGGCTGATGCCTGGACCTGGACTGCTGG | |
| sequence; | CCATGCCTACAACAGTGGATGGCTGTGTGCGGACCCCTAGCCT | |
| lowercase = | GGTCATCAACGATCTGATCTACGCCTACACCAGCAACCTGATC | |
| polyA tail | ACCAGAGGCTGCCAGGATATCGGCAAGAGCTACCAGGTGCTG | |
| (Amino acid | CAGATCGGCATCATCACCGTGAACAGCGATCTGGTGCCCGAC | |
| SEQ ID NO: | CTGAATCCTCGGATCAGCCACACCTTCAACATCAACGACAACC | |
| 724) | GGAAGTCCTGTTCTCTGGCCCTGCTGAACACCGACGTGTACCA | |
| | GCTGTGTAGCACCCCTAAGGTGGACGAGAGAAGCGACTATGC | |
| | CAGCAGCGGCATCGAGGATATCGTGCTGGACATCGTGAACTAC | |
| | GACGGCAGCATCAGCACCACCAGATTCAAGAACAACAACATCA | |
| | GCTTCGACCAGCCTTACGCCGCACTGTACCCTAGTGTTGGCCC | |
| | TGGCATCTACTACAAGGGCAAGATCATCTTCCTCGGCTACGGC | |
| | GGCCTGGAACACCCCATCAATGAGAACGCCATCTGCAACACCA | |
| | CCGGCTGTCCTGGCAAGACCCAGAGAGACTGCAATCAGGCCA | |
| | GCCACTCTCCATGGTTCAGCGACAGACGGATGGTCAACTCTAT | |
| | CATCGTGGTGGACAAGGGCCTGAACAGCGTGCCCAAGCTGAA | |
| | AGTGTGGACAATCAGCATGCGGCAGAACTACTGGGGCAGCGA | |
| | AGGCAGACTGCTGCTGCTGGGAAACAAGATCTACATCTACACC | |
| | CGGTCCACCAGCTGGCACAGCAAACTGCAGCTGGGAATCATC | |
| | GACATCACCGACTACAGCGACATCCGGATCAAGTGGACCTGG | |
| | CACAACGTGCTGAGCAGACCCGGCAACAATGAGTGTCCTTGG | |
| | GGCCACTCTTGCCCCGATGGATGTATCACCGGCGTGTACACC | |
| | GACGCCTATCCACTGAATCCTACCGGCTCCATCGTGTCCAGCG | |
| | TGATCCTGGACAGCCAGAAAAGCAGAGTGAACCCCGTGATCAC | |
| | ATACAGCACCGCCACCGAGAGAGTGAACGAGCTGGCCATCAG | |
| | AAACAAGACCCTGAGCGCCGGCTACACCACCACAAGCTGCATC | |
| | ACACACTACAACAAGGGCTACTGCTTCCACATCGTCGAGATCA | |
| | ACCACAAGTCCCTGAACACCTTCCAGCCTATGCTGTTCAAGAC | |
| | AGAGATCCCCAAGAGCTGCTCCTGATGAGCTCGCTTTCTTGCT | |
| | GTCCAATTTCTATTAAAGGTTCCTTTGTTCCCTAAGTCCAACTA | |
| | CTAAACTGGGGGATATTATGAAGGGCCTTGAGCATCTGGATT | |
| | CTGCCTAATAAAAAACATTTATTTTCATTGCAAaaaaaaaaaaaaa | |
| | aaaaaaaaaaaaaaaaaaaaaaaaaaaaaaaaaaaaaaaaaaaaaaaaaaaaaa | |
| | aaaaaaaaaaaaa | |
| PIV3224_5UTR_ | AGGAGGACTGGGCGAACCTGCATAGTGATCATAAGGTCATCA | 731 |
| 579/3UTR_ | TAGCCACCATGGAATACTGGAAGCACACCAACCACGGCAAGG | |
| 2 80A | ACGCCGGCAATGAGCTGGAAACAAGCACAGCTACCCACGGCA | |
| PIV3 HN | ACAAGCTGACCAACAAGATCACCTACATCCTGTGGACCATCAC | |
| PIV3224 | ACTGGTGCTGCTGAGCATCGTGTTCATCATCGTGCTGACCAAT | |
| DNA | AGCATCAAGAGCGACCTGATTCAGAGCGGCGTGAACACCCGG | |
| Underline = | CTGCTGACAATTCAGAGCCACGTGCAGAACTATATCCCCATCA | |
| 5′ cap; bold = | GCCTGACACAGCAGATCAGCGACCTGCGGAAGTTCATCAGCG | |
| 5′UTR | AGATCACCATCCGGAACGACAATCAAGAGGTGCCACCACAGC | |
| (5UTR_579) | GGATCACCCACGATGTGGGAATCAAGCCTCTGAACCCCGACG | |
| and 3′ UTR | ACTTCTGGCGGTGTACATCTGGCCTGCCTAGCCTGATGAAGAC | |
| (3UTR_2); | CCCTAAGATCCGGCTGATGCCTGGACCTGGACTGCTGGCCAT | |
| italics = | GCCTACAACAGTGGATGGCTGTGTGCGGACCCCTAGCCTGGT | |
| KOZAK | CATCAACGATCTGATCTACGCCTACACCAGCAACCTGATCACC | |
| sequence; | AGAGGCTGCCAGGATATCGGCAAGAGCTACCAGGTGCTGCAG | |
| lowercase = | ATCGGCATCATCACCGTGAACAGCGATCTGGTGCCCGACCTGA | |
| polyA tail | ATCCTCGGATCAGCCACACCTTCAACATCAACGACAACCGGAA | |
| (Amino acid | GTCCTGTTCTCTGGCCCTGCTGAACACCGACGTGTACCAGCTG | |
| SEQ ID NO: | TGTAGCACCCCTAAGGTGGACGAGAGAAGCGACTATGCCAGC | |
| 725) | AGCGGCATCGAGGATATCGTGCTGGACATCGTGAACTACGAC | |
| | GGCAGCATCAGCACCACCAGATTCAAGAACAACAACATCAGCT | |
| | TCGACCAGCCTTACGCCGCACTGTACCCTAGTGTTGGCCCTGG | |
| | CATCTACTACAAGGGCAAGATCATCTTCCTCGGCTACGGCGGC | |
| | CTGGAACACCCCATCAATGAGAACGCCATCTGCAACACCACCG | |
| | GCTGTCCTGGCAAGACCCAGAGAGACTGCAATCAGGCCAGCC | |
| | ACTCTCCATGGTTCAGCGACAGACGGATGGTCAACTCTATCAT | |
| | CGTGGTGGACAAGGGCCTGAACAGCGTGCCCAAGCTGAAAGT | |
| | GTGGACAATCAGCATGCGGCAGAACTACTGGGGCAGCGAAGG | |
| | CAGACTGCTGCTGCTGGGAAACAAGATCTACATCTACACCCGG | |
| | TCCACCAGCTGGCACAGCAAACTGCAGCTGGGAATCATCGACA | |
| | TCACCGACTACAGCGACATCCGGATCAAGTGGACCTGGCACAA | |
| | CGTGCTGAGCAGACCCGGCAACAATGAGTGTCCTTGGGGCCA | |
| | CTCTTGCCCCGATGGATGTATCACCGGCGTGTACACCGACGCC | |
| | TATCCACTGAATCCTACCGGCTCCATCGTGTCCAGCGTGATCC | |
| | TGGACAGCCAGAAAAGCAGAGTGAACCCCGTGATCACATACAG | |
| | CACCGCCACCGAGAGAGTGAACGAGCTGGCCATCAGAAACAA | |
| | GACCCTGAGCGCCGGCTACACCACCACAAGCTGCATCACACA | |
| | CTACAACAAGGGCTACTGCTTCCACATCGTCGAGATCAACCAC | |
| | AAGTCCCTGAACACCTTCCAGCCTATGCTGTTCAAGACAGAGA | |
| | TCCCCAAGAGCTGCTCCTGATGAGCTCGCTTTCTTGCTGTCCA | |
| | ATTTCTATTAAAGGTTCCTTTGTTCCCTAAGTCCAACTACTAAA | |
| | CTGGGGGATATTATGAAGGGCCTTGAGCATCTGGATTCTGCC | |
| | TAATAAAAAACATTTATTTTCATTGCAAaaaaaaaaaaaaaaaaaaaa | |
| | aaaaaaaaaaaaaaaaaaaaaaaaaaaaaaaaaaaaaaaaaaaaaaaaaaaaaa | |
| | aaaaaa | |
| PIV3225_5U | AGGAGGACTGGGCGAACCTGCATAGTGATCATAAGGTCATCA | 732 |
| TR_579/3UT | TAGCCACCATGGAATACTGGAAGCACACCAACCACGGCAAGG | |
| R_2 80A | ACGCCGGCAATGAGCTGGAAACAAGCACAGCTACCCACGGCA | |
| PIV3 HN | ACAAGCTGACCAACAAGATCACCTACATCCTGTGGACCATCAC | |
| PIV3225 | ACTGGTGCTGCTGAGCATCGTGTTCATCATCGTGCTGACCAAT | |
| DNA | AGCATCAAGAGCCGGAACGACAATCAAGAGGTGCCACCACAG | |
| Underline = | CGGATCACCCACGATGTGGGAATCAAGCCTCTGAACCCCGAC | |
| 5′ cap; bold = | GACTTCTGGCGGTGTACATCTGGCCTGCCTAGCCTGATGAAGA | |
| 5′UTR | CCCCTAAGATCCGGCTGATGCCTGGACCTGGACTGCTGGCCA | |
| (5UTR_579) | TGCCTACAACAGTGGATGGCTGTGTGCGGACCCCTAGCCTGG | |
| and 3′ UTR | TCATCAACGATCTGATCTACGCCTACACCAGCAACCTGATCAC | |
| (3UTR_2); | CAGAGGCTGCCAGGATATCGGCAAGAGCTACCAGGTGCTGCA | |
| italics = | GATCGGCATCATCACCGTGAACAGCGATCTGGTGCCCGACCT | |
| KOZAK | GAATCCTCGGATCAGCCACACCTTCAACATCAACGACAACCGG | |
| sequence; | AAGTCCTGTTCTCTGGCCCTGCTGAACACCGACGTGTACCAGC | |
| lowercase = | TGTGTAGCACCCCTAAGGTGGACGAGAGAAGCGACTATGCCA | |
| (Amino acid | GCAGCGGCATCGAGGATATCGTGCTGGACATCGTGAACTACG | |
| SEQ ID NO: | ACGGCAGCATCAGCACCACCAGATTCAAGAACAACAACATCAG | |
| 726) | CTTCGACCAGCCTTACGCCGCACTGTACCCTAGTGTTGGCCCT | |
| | GGCATCTACTACAAGGGCAAGATCATCTTCCTCGGCTACGGCG | |
| | GCCTGGAACACCCCATCAATGAGAACGCCATCTGCAACACCAC | |
| | CGGCTGTCCTGGCAAGACCCAGAGAGACTGCAATCAGGCCAG | |
| | CCACTCTCCATGGTTCAGCGACAGACGGATGGTCAACTCTATC | |
| | ATCGTGGTGGACAAGGGCCTGAACAGCGTGCCCAAGCTGAAA | |
| | GTGTGGACAATCAGCATGCGGCAGAACTACTGGGGCAGCGAA | |
| | GGCAGACTGCTGCTGCTGGGAAACAAGATCTACATCTACACCC | |
| | GGTCCACCAGCTGGCACAGCAAACTGCAGCTGGGAATCATCG | |
| | ACATCACCGACTACAGCGACATCCGGATCAAGTGGACCTGGCA | |
| | CAACGTGCTGAGCAGACCCGGCAACAATGAGTGTCCTTGGGG | |
| | CCACTCTTGCCCCGATGGATGTATCACCGGCGTGTACACCGAC | |
| | GCCTATCCACTGAATCCTACCGGCTCCATCGTGTCCAGCGTGA | |
| | TCCTGGACAGCCAGAAAAGCAGAGTGAACCCCGTGATCACATA | |
| | CAGCACCGCCACCGAGAGAGTGAACGAGCTGGCCATCAGAAA | |
| | CAAGACCCTGAGCGCCGGCTACACCACCACAAGCTGCATCAC | |
| | ACACTACAACAAGGGCTACTGCTTCCACATCGTCGAGATCAAC | |
| | CACAAGTCCCTGAACACCTTCCAGCCTATGCTGTTCAAGACAG | |
| | AGATCCCCAAGAGCTGCTCCTGATGAGCTCGCTTTCTTGCTGT | |
| | CCAATTTCTATTAAAGGTTCCTTTGTTCCCTAAGTCCAACTACT | |
| | AAACTGGGGGATATTATGAAGGGCCTTGAGCATCTGGATTCT | |
| | GCCTAATAAAAAACATTTATTTTCATTGCAAaaaaaaaaaaaaaaaa | |
| | aaaaaaaaaaaaaaaaaaaaaaaaaaaaaaaaaaaaaaaaaaaaaaaaaaaaaa | |
| | aaaaaaaaaa | |
| PIV3223_5UTR_ | AGGTGTCAGAGTTTAACTTGAAGACTATTTCTAGGGATAATAT | 733 |
| 582/3UTR_ | CCCTGCCACCATGGAATACTGGAAGCACACCAACCACGGCAA | |
| 2 80A | GGACGCCGGCAATGAGCTGGAAACAAGCACAGCTACCCACGG | |
| PIV3 HN WT | CAACAAGCTGACCAACAAGATCACCTACATCCTGTGGACCATC | |
| PIV3223 | ACACTGGTGCTGCTGAGCATCGTGTTCATCATCGTGCTGACCA | |
| DNA | ATAGCATCAAGAGCGAGAAGGCCAGAGAGAGCCTGCTGCAGG | |
| Underline = | ACATCAACAACGAGTTCATGGAAGTGACCGAGAAGATCCAGGT | |
| 5′ cap; bold = | GGCCAGCGACAACACCAACGACCTGATTCAGAGCGGCGTGAA | |
| 5′UTR | CACCCGGCTGCTGACAATTCAGAGCCACGTGCAGAACTATATC | |
| (5UTR_582) | CCCATCAGCCTGACACAGCAGATCAGCGACCTGCGGAAGTTCA | |
| and 3′ UTR | TCAGCGAGATCACCATCCGGAACGACAATCAAGAGGTGCCACC | |
| (3UTR_2); | ACAGCGGATCACCCACGATGTGGGAATCAAGCCTCTGAACCCC | |
| italics = | GACGACTTCTGGCGGTGTACATCTGGCCTGCCTAGCCTGATGA | |
| KOZAK | AGACCCCTAAGATCCGGCTGATGCCTGGACCTGGACTGCTGG | |
| sequence; | CCATGCCTACAACAGTGGATGGCTGTGTGCGGACCCCTAGCCT | |
| lowercase = | GGTCATCAACGATCTGATCTACGCCTACACCAGCAACCTGATC | |
| polyA tail | ACCAGAGGCTGCCAGGATATCGGCAAGAGCTACCAGGTGCTG | |
| (Amino acid | CAGATCGGCATCATCACCGTGAACAGCGATCTGGTGCCCGAC | |
| SEQ ID NO: | CTGAATCCTCGGATCAGCCACACCTTCAACATCAACGACAACC | |
| 724) | GGAAGTCCTGTTCTCTGGCCCTGCTGAACACCGACGTGTACCA | |
| | GCTGTGTAGCACCCCTAAGGTGGACGAGAGAAGCGACTATGC | |
| | CAGCAGCGGCATCGAGGATATCGTGCTGGACATCGTGAACTAC | |
| | GACGGCAGCATCAGCACCACCAGATTCAAGAACAACAACATCA | |
| | GCTTCGACCAGCCTTACGCCGCACTGTACCCTAGTGTTGGCCC | |
| | TGGCATCTACTACAAGGGCAAGATCATCTTCCTCGGCTACGGC | |
| | GGCCTGGAACACCCCATCAATGAGAACGCCATCTGCAACACCA | |
| | CCGGCTGTCCTGGCAAGACCCAGAGAGACTGCAATCAGGCCA | |
| | GCCACTCTCCATGGTTCAGCGACAGACGGATGGTCAACTCTAT | |
| | CATCGTGGTGGACAAGGGCCTGAACAGCGTGCCCAAGCTGAA | |
| | AGTGTGGACAATCAGCATGCGGCAGAACTACTGGGGCAGCGA | |
| | AGGCAGACTGCTGCTGCTGGGAAACAAGATCTACATCTACACC | |
| | CGGTCCACCAGCTGGCACAGCAAACTGCAGCTGGGAATCATC | |
| | GACATCACCGACTACAGCGACATCCGGATCAAGTGGACCTGG | |
| | CACAACGTGCTGAGCAGACCCGGCAACAATGAGTGTCCTTGG | |
| | GGCCACTCTTGCCCCGATGGATGTATCACCGGCGTGTACACC | |
| | GACGCCTATCCACTGAATCCTACCGGCTCCATCGTGTCCAGCG | |
| | TGATCCTGGACAGCCAGAAAAGCAGAGTGAACCCCGTGATCAC | |
| | ATACAGCACCGCCACCGAGAGAGTGAACGAGCTGGCCATCAG | |
| | AAACAAGACCCTGAGCGCCGGCTACACCACCACAAGCTGCATC | |
| | ACACACTACAACAAGGGCTACTGCTTCCACATCGTCGAGATCA | |
| | ACCACAAGTCCCTGAACACCTTCCAGCCTATGCTGTTCAAGAC | |
| | AGAGATCCCCAAGAGCTGCTCCTGATGAGCTCGCTTTCTTGCT | |
| | GTCCAATTTCTATTAAAGGTTCCTTTGTTCCCTAAGTCCAACTA | |
| | CTAAACTGGGGGATATTATGAAGGGCCTTGAGCATCTGGATT | |
| | CTGCCTAATAAAAAACATTTATTTTCATTGCAAaaaaaaaaaaaaa | |
| | aaaaaaaaaaaaaaaaaaaaaaaaaaaaaaaaaaaaaaaaaaaaaaaaaaaaaa | |
| | aaaaaaaaaaaaa | |
| PIV3224_5UTR_ | AGGTGTCAGAGTTTAACTTGAAGACTATTTCTAGGGATAATAT | 734 |
| 582/3UTR_ | CCCTGCCACCATGGAATACTGGAAGCACACCAACCACGGCAA | |
| 2 80A | GGACGCCGGCAATGAGCTGGAAACAAGCACAGCTACCCACGG | |
| PIV3 HN | CAACAAGCTGACCAACAAGATCACCTACATCCTGTGGACCATC | |
| PIV3224 | ACACTGGTGCTGCTGAGCATCGTGTTCATCATCGTGCTGACCA | |
| DNA | ATAGCATCAAGAGCGACCTGATTCAGAGCGGCGTGAACACCC | |
| Underline = | GGCTGCTGACAATTCAGAGCCACGTGCAGAACTATATCCCCAT | |
| 5′ cap; bold = | CAGCCTGACACAGCAGATCAGCGACCTGCGGAAGTTCATCAG | |
| 5′UTR | CGAGATCACCATCCGGAACGACAATCAAGAGGTGCCACCACA | |
| (5UTR_582) | GCGGATCACCCACGATGTGGGAATCAAGCCTCTGAACCCCGA | |
| and 3′ UTR | CGACTTCTGGCGGTGTACATCTGGCCTGCCTAGCCTGATGAAG | |
| (3UTR_2); | ACCCCTAAGATCCGGCTGATGCCTGGACCTGGACTGCTGGCC | |
| italics = | ATGCCTACAACAGTGGATGGCTGTGTGCGGACCCCTAGCCTG | |
| KOZAK | GTCATCAACGATCTGATCTACGCCTACACCAGCAACCTGATCA | |
| sequence; | CCAGAGGCTGCCAGGATATCGGCAAGAGCTACCAGGTGCTGC | |
| lowercase = | AGATCGGCATCATCACCGTGAACAGCGATCTGGTGCCCGACCT | |
| polyA tail | GAATCCTCGGATCAGCCACACCTTCAACATCAACGACAACCGG | |
| (Amino acid | AAGTCCTGTTCTCTGGCCCTGCTGAACACCGACGTGTACCAGC | |
| SEQ ID NO: | TGTGTAGCACCCCTAAGGTGGACGAGAGAAGCGACTATGCCA | |
| 725) | GCAGCGGCATCGAGGATATCGTGCTGGACATCGTGAACTACG | |
| | ACGGCAGCATCAGCACCACCAGATTCAAGAACAACAACATCAG | |
| | CTTCGACCAGCCTTACGCCGCACTGTACCCTAGTGTTGGCCCT | |
| | GGCATCTACTACAAGGGCAAGATCATCTTCCTCGGCTACGGCG | |
| | GCCTGGAACACCCCATCAATGAGAACGCCATCTGCAACACCAC | |
| | CGGCTGTCCTGGCAAGACCCAGAGAGACTGCAATCAGGCCAG | |
| | CCACTCTCCATGGTTCAGCGACAGACGGATGGTCAACTCTATC | |
| | ATCGTGGTGGACAAGGGCCTGAACAGCGTGCCCAAGCTGAAA | |
| | GTGTGGACAATCAGCATGCGGCAGAACTACTGGGGCAGCGAA | |
| | GGCAGACTGCTGCTGCTGGGAAACAAGATCTACATCTACACCC | |
| | GGTCCACCAGCTGGCACAGCAAACTGCAGCTGGGAATCATCG | |
| | ACATCACCGACTACAGCGACATCCGGATCAAGTGGACCTGGCA | |
| | CAACGTGCTGAGCAGACCCGGCAACAATGAGTGTCCTTGGGG | |
| | CCACTCTTGCCCCGATGGATGTATCACCGGCGTGTACACCGAC | |
| | GCCTATCCACTGAATCCTACCGGCTCCATCGTGTCCAGCGTGA | |
| | TCCTGGACAGCCAGAAAAGCAGAGTGAACCCCGTGATCACATA | |
| | CAGCACCGCCACCGAGAGAGTGAACGAGCTGGCCATCAGAAA | |
| | CAAGACCCTGAGCGCCGGCTACACCACCACAAGCTGCATCAC | |
| | ACACTACAACAAGGGCTACTGCTTCCACATCGTCGAGATCAAC | |
| | CACAAGTCCCTGAACACCTTCCAGCCTATGCTGTTCAAGACAG | |
| | AGATCCCCAAGAGCTGCTCCTGATGAGCTCGCTTTCTTGCTGT | |
| | CCAATTTCTATTAAAGGTTCCTTTGTTCCCTAAGTCCAACTACT | |
| | AAACTGGGGGATATTATGAAGGGCCTTGAGCATCTGGATTCT | |
| | GCCTAATAAAAAACATTTATTTTCATTGCAAaaaaaaaaaaaaaaaa | |
| | aaaaaaaaaaaaaaaaaaaaaaaaaaaaaaaaaaaaaaaaaaaaaaaaaaaaaa | |
| | aaaaaaaaaa | |
| PIV3225_5UTR_ | AGGTGTCAGAGTTTAACTTGAAGACTATTTCTAGGGATAATAT | 735 |
| 582/3UTR_ | CCCTGCCACCATGGAATACTGGAAGCACACCAACCACGGCAA | |
| 2 80A | GGACGCCGGCAATGAGCTGGAAACAAGCACAGCTACCCACGG | |
| PIV3 HN | CAACAAGCTGACCAACAAGATCACCTACATCCTGTGGACCATC | |
| PIV3225 | ACACTGGTGCTGCTGAGCATCGTGTTCATCATCGTGCTGACCA | |
| DNA | ATAGCATCAAGAGCCGGAACGACAATCAAGAGGTGCCACCACA | |
| Underline = | GCGGATCACCCACGATGTGGGAATCAAGCCTCTGAACCCCGA | |
| 5′ cap; bold = | CGACTTCTGGCGGTGTACATCTGGCCTGCCTAGCCTGATGAAG | |
| 5′UTR | ACCCCTAAGATCCGGCTGATGCCTGGACCTGGACTGCTGGCC | |
| (5UTR_582) | ATGCCTACAACAGTGGATGGCTGTGTGCGGACCCCTAGCCTG | |
| and 3′ UTR | GTCATCAACGATCTGATCTACGCCTACACCAGCAACCTGATCA | |
| (3UTR_2); | CCAGAGGCTGCCAGGATATCGGCAAGAGCTACCAGGTGCTGC | |
| italics = | AGATCGGCATCATCACCGTGAACAGCGATCTGGTGCCCGACCT | |
| KOZAK | GAATCCTCGGATCAGCCACACCTTCAACATCAACGACAACCGG | |
| sequence; | AAGTCCTGTTCTCTGGCCCTGCTGAACACCGACGTGTACCAGC | |
| lowercase = | TGTGTAGCACCCCTAAGGTGGACGAGAGAAGCGACTATGCCA | |
| polyA tail | GCAGCGGCATCGAGGATATCGTGCTGGACATCGTGAACTACG | |
| (Amino acid | ACGGCAGCATCAGCACCACCAGATTCAAGAACAACAACATCAG | |
| SEQ ID NO: | CTTCGACCAGCCTTACGCCGCACTGTACCCTAGTGTTGGCCCT | |
| 726) | GGCATCTACTACAAGGGCAAGATCATCTTCCTCGGCTACGGCG | |
| | GCCTGGAACACCCCATCAATGAGAACGCCATCTGCAACACCAC | |
| | CGGCTGTCCTGGCAAGACCCAGAGAGACTGCAATCAGGCCAG | |
| | CCACTCTCCATGGTTCAGCGACAGACGGATGGTCAACTCTATC | |
| | ATCGTGGTGGACAAGGGCCTGAACAGCGTGCCCAAGCTGAAA | |
| | GTGTGGACAATCAGCATGCGGCAGAACTACTGGGGCAGCGAA | |
| | GGCAGACTGCTGCTGCTGGGAAACAAGATCTACATCTACACCC | |
| | GGTCCACCAGCTGGCACAGCAAACTGCAGCTGGGAATCATCG | |
| | ACATCACCGACTACAGCGACATCCGGATCAAGTGGACCTGGCA | |
| | CAACGTGCTGAGCAGACCCGGCAACAATGAGTGTCCTTGGGG | |
| | CCACTCTTGCCCCGATGGATGTATCACCGGCGTGTACACCGAC | |
| | GCCTATCCACTGAATCCTACCGGCTCCATCGTGTCCAGCGTGA | |
| | TCCTGGACAGCCAGAAAAGCAGAGTGAACCCCGTGATCACATA | |
| | CAGCACCGCCACCGAGAGAGTGAACGAGCTGGCCATCAGAAA | |
| | CAAGACCCTGAGCGCCGGCTACACCACCACAAGCTGCATCAC | |
| | ACACTACAACAAGGGCTACTGCTTCCACATCGTCGAGATCAAC | |
| | CACAAGTCCCTGAACACCTTCCAGCCTATGCTGTTCAAGACAG | |
| | AGATCCCCAAGAGCTGCTCCTGATGAGCTCGCTTTCTTGCTGT | |
| | CCAATTTCTATTAAAGGTTCCTTTGTTCCCTAAGTCCAACTACT | |
| | AAACTGGGGGATATTATGAAGGGCCTTGAGCATCTGGATTCT | |
| | GCCTAATAAAAAACATTTATTTTCATTGCAAaaaaaaaaaaaaaaaa | |
| | aaaaaaaaaaaaaaaaaaaaaaaaaaaaaaaaaaaaaaaaaaaaaaaaaaaaaa | |
| | aaaaaaaaaa | |

TABLE 18
PIV3 HN RNA

| | | SEQ ID |
|---|---|---|
| ID | | NO: |
| PIV3 WT HN PIV3223 | | 736 |
| UGA stop codon; (Amino acid SEQ ID NO: 724) | | |
| PIV3 HN PIV3224 | | 737 |
| UGA stop codon; (Amino acid SEQ ID NO: 725) | | |
| PIV3 HN PIV3225 | | 738 |
| UGA stop codon; (Amino acid SEQ ID NO: 726) | | |
| | | SEQ ID |
| ID | Sequence | NO: |
| PIV3223_5UTR_ | AGGAGGACUGGGCGAACCUGCAUAGUGAUCAUAAGGUCA | 739 |
| 579/3UTR_ | UCAUAGCCACCAUGGAAUACUGGAAGCACACCAACCACGG | |
| 2 80A_ | CAAGGACGCCGGCAAUGAGCUGGAAACAAGCACAGCUACC | |
| modRNA | CACGGCAACAAGCUGACCAACAAGAUCACCUACAUCCUGU | |
| Underline = | GGACCAUCACACUGGUGCUGCUGAGCAUCGUGUUCAUCAU | |
| 5′ cap; bold = | CGUGCUGACCAAUAGCAUCAAGAGCGAGAAGGCCAGAGAG | |
| 5′UTR | AGCCUGCUGCAGGACAUCAACAACGAGUUCAUGGAAGUGA | |
| (5UTR_579) | CCGAGAAGAUCCAGGUGGCCAGCGACAACACCAACGACCU | |
| and 3′ UTR | GAUUCAGAGCGGCGUGAACACCCGGCUGCUGACAAUUCAG | |
| (3UTR_2); | AGCCACGUGCAGAACUAUAUCCCCAUCAGCCUGACACAGC | |
| italics = | AGAUCAGCGACCUGCGGAAGUUCAUCAGCGAGAUCACCAU | |
| KOZAK | CCGGAACGACAAUCAAGAGGUGCCACCACAGCGGAUCACC | |
| sequence; | CACGAUGUGGGAAUCAAGCCUCUGAACCCCGACGACUUCU | |
| lowercase = | GGCGGUGUACAUCUGGCCUGCCUAGCCUGAUGAAGACCC | |
| polyA tail | CUAAGAUCCGGCUGAUGCCUGGACCUGGACUGCUGGCCA | |
| (Amino acid | UGCCUACAACAGUGGAUGGCUGUGUGCGGACCCCUAGCC | |
| SEQ ID NO: | UGGUCAUCAACGAUCUGAUCUACGCCUACACCAGCAACCU | |
| 724) | GAUCACCAGAGGCUGCCAGGAUAUCGGCAAGAGCUACCAG | |
| | GUGCUGCAGAUCGGCAUCAUCACCGUGAACAGCGAUCUG | |
| | GUGCCCGACCUGAAUCCUCGGAUCAGCCACACCUUCAACA | |
| | UCAACGACAACCGGAAGUCCUGUUCUCUGGCCCUGCUGAA | |
| | CACCGACGUGUACCAGCUGUGUAGCACCCCUAAGGUGGAC | |
| | GAGAGAAGCGACUAUGCCAGCAGCGGCAUCGAGGAUAUCG | |
| | UGCUGGACAUCGUGAACUACGACGGCAGCAUCAGCACCAC | |
| | CAGAUUCAAGAACAACAACAUCAGCUUCGACCAGCCUUAC | |
| | GCCGCACUGUACCCUAGUGUUGGCCCUGGCAUCUACUACA | |
| | AGGGCAAGAUCAUCUUCCUCGGCUACGGCGGCCUGGAAC | |
| | ACCCCAUCAAUGAGAACGCCAUCUGCAACACCACCGGCUG | |
| | UCCUGGCAAGACCCAGAGAGACUGCAAUCAGGCCAGCCAC | |
| | UCUCCAUGGUUCAGCGACAGACGGAUGGUCAACUCUAUCA | |
| | UCGUGGUGGACAAGGGCCUGAACAGCGUGCCCAAGCUGA | |
| | AAGUGUGGACAAUCAGCAUGCGGCAGAACUACUGGGGCAG | |
| | CGAAGGCAGACUGCUGCUGCUGGGAAACAAGAUCUACAUC | |
| | UACACCCGGUCCACCAGCUGGCACAGCAAACUGCAGCUGG | |
| | GAAUCAUCGACAUCACCGACUACAGCGACAUCCGGAUCAA | |
| | GUGGACCUGGCACAACGUGCUGAGCAGACCCGGCAACAAU | |
| | GAGUGUCCUUGGGGCCACUCUUGCCCCGAUGGAUGUAUC | |
| | ACCGGCGUGUACACCGACGCCUAUCCACUGAAUCCUACCG | |
| | GCUCCAUCGUGUCCAGCGUGAUCCUGGACAGCCAGAAAAG | |
| | CAGAGUGAACCCCGUGAUCACAUACAGCACCGCCACCGAG | |
| | AGAGUGAACGAGCUGGCCAUCAGAAACAAGACCCUGAGCG | |
| | CCGGCUACACCACCACAAGCUGCAUCACACACUACAACAA | |
| | GGGCUACUGCUUCCACAUCGUCGAGAUCAACCACAAGUCC | |
| | CUGAACACCUUCCAGCCUAUGCUGUUCAAGACAGAGAUCC | |
| | CCAAGAGCUGCUCCUGAUGAGCUCGCUUUCUUGCUGUCC | |
| | AAUUUCUAUUAAAGGUUCCUUUGUUCCCUAAGUCCAACU | |
| | ACUAAACUGGGGGAUAUUAUGAAGGGCCUUGAGCAUCUG | |
| | GAUUCUGCCUAAUAAAAAACAUUUAUUUUCAUUGCAAaaa | |
| | aaaaaaaaaaaaaaaaaaaaaaaaaaaaaaaaaaaaaaaaaaaaaaaaaaaa | |
| | aaaaaaaaaaaaaaaaaaaaaaaaa | |
| PIV3224_5UTR_ | AGGAGGACUGGGCGAACCUGCAUAGUGAUCAUAAGGUCA | 740 |
| 579/3UTR_ | UCAUAGCCACCAUGGAAUACUGGAAGCACACCAACCACGG | |
| 2 80A_ | CAAGGACGCCGGCAAUGAGCUGGAAACAAGCACAGCUACC | |
| modRNA | CACGGCAACAAGCUGACCAACAAGAUCACCUACAUCCUGU | |
| Underline = | GGACCAUCACACUGGUGCUGCUGAGCAUCGUGUUCAUCAU | |
| 5′ cap; bold = | CGUGCUGACCAAUAGCAUCAAGAGCGACCUGAUUCAGAGC | |
| 5′ UTR | GGCGUGAACACCCGGCUGCUGACAAUUCAGAGCCACGUG | |
| (5UTR_579) | CAGAACUAUAUCCCCAUCAGCCUGACACAGCAGAUCAGCG | |
| and 3′ UTR | ACCUGCGGAAGUUCAUCAGCGAGAUCACCAUCCGGAACGA | |
| (3UTR_2); | CAAUCAAGAGGUGCCACCACAGCGGAUCACCCACGAUGUG | |
| italics = | GGAAUCAAGCCUCUGAACCCCGACGACUUCUGGCGGUGUA | |
| KOZAK | CAUCUGGCCUGCCUAGCCUGAUGAAGACCCCUAAGAUCCG | |
| sequence; | GCUGAUGCCUGGACCUGGACUGCUGGCCAUGCCUACAAC | |
| lowercase = | AGUGGAUGGCUGUGUGCGGACCCCUAGCCUGGUCAUCAA | |
| play tail | CGAUCUGAUCUACGCCUACACCAGCAACCUGAUCACCAGA | |
| (Amino acid | GGCUGCCAGGAUAUCGGCAAGAGCUACCAGGUGCUGCAG | |
| SEQ ID NO: | AUCGGCAUCAUCACCGUGAACAGCGAUCUGGUGCCCGACC | |
| 725) | UGAAUCCUCGGAUCAGCCACACCUUCAACAUCAACGACAA | |
| | CCGGAAGUCCUGUUCUCUGGCCCUGCUGAACACCGACGU | |
| | GUACCAGCUGUGUAGCACCCCUAAGGUGGACGAGAGAAGC | |
| | GACUAUGCCAGCAGCGGCAUCGAGGAUAUCGUGCUGGAC | |
| | AUCGUGAACUACGACGGCAGCAUCAGCACCACCAGAUUCA | |
| | AGAACAACAACAUCAGCUUCGACCAGCCUUACGCCGCACU | |
| | GUACCCUAGUGUUGGCCCUGGCAUCUACUACAAGGGCAAG | |
| | AUCAUCUUCCUCGGCUACGGCGGCCUGGAACACCCCAUCA | |
| | AUGAGAACGCCAUCUGCAACACCACCGGCUGUCCUGGCAA | |
| | GACCCAGAGAGACUGCAAUCAGGCCAGCCACUCUCCAUGG | |
| | UUCAGCGACAGACGGAUGGUCAACUCUAUCAUCGUGGUG | |
| | GACAAGGGCCUGAACAGCGUGCCCAAGCUGAAAGUGUGGA | |
| | CAAUCAGCAUGCGGCAGAACUACUGGGGCAGCGAAGGCAG | |
| | ACUGCUGCUGCUGGGAAACAAGAUCUACAUCUACACCCGG | |
| | UCCACCAGCUGGCACAGCAAACUGCAGCUGGGAAUCAUCG | |
| | ACAUCACCGACUACAGCGACAUCCGGAUCAAGUGGACCUG | |
| | GCACAACGUGCUGAGCAGACCCGGCAACAAUGAGUGUCCU | |
| | UGGGGCCACUCUUGCCCCGAUGGAUGUAUCACCGGCGUG | |
| | UACACCGACGCCUAUCCACUGAAUCCUACCGGCUCCAUCG | |
| | UGUCCAGCGUGAUCCUGGACAGCCAGAAAAGCAGAGUGAA | |
| | CCCCGUGAUCACAUACAGCACCGCCACCGAGAGAGUGAAC | |
| | GAGCUGGCCAUCAGAAACAAGACCCUGAGCGCCGGCUACA | |
| | CCACCACAAGCUGCAUCACACACUACAACAAGGGCUACUG | |
| | CUUCCACAUCGUCGAGAUCAACCACAAGUCCCUGAACACC | |
| | UUCCAGCCUAUGCUGUUCAAGACAGAGAUCCCCAAGAGCU | |
| | GCUCCUGAUGAGCUCGCUUUCUUGCUGUCCAAUUUCUAU | |
| | UAAAGGUUCCUUUGUUCCCUAAGUCCAACUACUAAACUG | |
| | GGGGAUAUUAUGAAGGGCCUUGAGCAUCUGGAUUCUGCC | |
| | UAAUAAAAAACAUUUAUUUUCAUUGCAAaaaaaaaaaaaaaaa | |
| | aaaaaaaaaaaaaaaaaaaaaaaaaaaaaaaaaaaaaaaaaaaaaaaaaaaa | |
| | aaaaaaaaaaaaa | |
| PIV3225_5UTR_ | AGGAGGACUGGGCGAACCUGCAUAGUGAUCAUAAGGUCA | 741 |
| 579/3UTR_ | UCAUAGCCACCAUGGAAUACUGGAAGCACACCAACCACGG | |
| 2 80A_ | CAAGGACGCCGGCAAUGAGCUGGAAACAAGCACAGCUACC | |
| modRNA | CACGGCAACAAGCUGACCAACAAGAUCACCUACAUCCUGU | |
| Underline = | GGACCAUCACACUGGUGCUGCUGAGCAUCGUGUUCAUCAU | |
| 5′ cap; bold = | CGUGCUGACCAAUAGCAUCAAGAGCCGGAACGACAAUCAA | |
| 5′ UTR | GAGGUGCCACCACAGCGGAUCACCCACGAUGUGGGAAUCA | |
| (5UTR_579) | AGCCUCUGAACCCCGACGACUUCUGGCGGUGUACAUCUG | |
| and 3′ UTR | GCCUGCCUAGCCUGAUGAAGACCCCUAAGAUCCGGCUGAU | |
| (3UTR_2); | GCCUGGACCUGGACUGCUGGCCAUGCCUACAACAGUGGA | |
| italics = | UGGCUGUGUGCGGACCCCUAGCCUGGUCAUCAACGAUCU | |
| KOZAK | GAUCUACGCCUACACCAGCAACCUGAUCACCAGAGGCUGC | |
| sequence; | CAGGAUAUCGGCAAGAGCUACCAGGUGCUGCAGAUCGGCA | |
| lowercase = | UCAUCACCGUGAACAGCGAUCUGGUGCCCGACCUGAAUCC | |
| play tail | UCGGAUCAGCCACACCUUCAACAUCAACGACAACCGGAAG | |
| (Amino acid | UCCUGUUCUCUGGCCCUGCUGAACACCGACGUGUACCAG | |
| SEQ ID NO: | CUGUGUAGCACCCCUAAGGUGGACGAGAGAAGCGACUAUG | |
| 726) | CCAGCAGCGGCAUCGAGGAUAUCGUGCUGGACAUCGUGA | |
| | ACUACGACGGCAGCAUCAGCACCACCAGAUUCAAGAACAA | |
| | CAACAUCAGCUUCGACCAGCCUUACGCCGCACUGUACCCU | |
| | AGUGUUGGCCCUGGCAUCUACUACAAGGGCAAGAUCAUCU | |
| | UCCUCGGCUACGGCGGCCUGGAACACCCCAUCAAUGAGAA | |
| | CGCCAUCUGCAACACCACCGGCUGUCCUGGCAAGACCCAG | |
| | AGAGACUGCAAUCAGGCCAGCCACUCUCCAUGGUUCAGCG | |
| | ACAGACGGAUGGUCAACUCUAUCAUCGUGGUGGACAAGGG | |
| | CCUGAACAGCGUGCCCAAGCUGAAAGUGUGGACAAUCAGC | |
| | AUGCGGCAGAACUACUGGGGCAGCGAAGGCAGACUGCUG | |
| | CUGCUGGGAAACAAGAUCUACAUCUACACCCGGUCCACCA | |
| | GCUGGCACAGCAAACUGCAGCUGGGAAUCAUCGACAUCAC | |
| | CGACUACAGCGACAUCCGGAUCAAGUGGACCUGGCACAAC | |
| | GUGCUGAGCAGACCCGGCAACAAUGAGUGUCCUUGGGGC | |
| | CACUCUUGCCCCGAUGGAUGUAUCACCGGCGUGUACACC | |
| | GACGCCUAUCCACUGAAUCCUACCGGCUCCAUCGUGUCCA | |
| | GCGUGAUCCUGGACAGCCAGAAAAGCAGAGUGAACCCCGU | |
| | GAUCACAUACAGCACCGCCACCGAGAGAGUGAACGAGCUG | |
| | GCCAUCAGAAACAAGACCCUGAGCGCCGGCUACACCACCA | |
| | CAAGCUGCAUCACACACUACAACAAGGGCUACUGCUUCCA | |
| | CAUCGUCGAGAUCAACCACAAGUCCCUGAACACCUUCCAG | |
| | CCUAUGCUGUUCAAGACAGAGAUCCCCAAGAGCUGCUCCU | |
| | GAUGAGCUCGCUUUCUUGCUGUCCAAUUUCUAUUAAAGG | |
| | UUCCUUUGUUCCCUAAGUCCAACUACUAAACUGGGGGAU | |
| | AUUAUGAAGGGCCUUGAGCAUCUGGAUUCUGCCUAAUAA | |
| | AAAACAUUUAUUUUCAUUGCAAaaaaaaaaaaaaaaaaaaaaaaa | |
| | aaaaaaaaaaaaaaaaaaaaaaaaaaaaaaaaaaaaaaaaaaaaaaaaaaaa | |
| | aaaaa | |
| PIV3223_5UTR_ | AGGUGUCAGAGUUUAACUUGAAGACUAUUUCUAGGGAUA | 742 |
| 582/3UTR_ | AUAUCCCUGCCACCAUGGAAUACUGGAAGCACACCAACCA | |
| 2 80A_ | CGGCAAGGACGCCGGCAAUGAGCUGGAAACAAGCACAGCU | |
| modRNA | ACCCACGGCAACAAGCUGACCAACAAGAUCACCUACAUCC | |
| Underline = | UGUGGACCAUCACACUGGUGCUGCUGAGCAUCGUGUUCA | |
| 5′ cap; bold = | UCAUCGUGCUGACCAAUAGCAUCAAGAGCGAGAAGGCCAG | |
| 5′UTR | AGAGAGCCUGCUGCAGGACAUCAACAACGAGUUCAUGGAA | |
| (5UTR_582) | GUGACCGAGAAGAUCCAGGUGGCCAGCGACAACACCAACG | |
| and 3′ UTR | ACCUGAUUCAGAGCGGCGUGAACACCCGGCUGCUGACAAU | |
| (3UTR_2); | UCAGAGCCACGUGCAGAACUAUAUCCCCAUCAGCCUGACA | |
| italics = | CAGCAGAUCAGCGACCUGCGGAAGUUCAUCAGCGAGAUCA | |
| KOZAK | CCAUCCGGAACGACAAUCAAGAGGUGCCACCACAGCGGAU | |
| sequence; | CACCCACGAUGUGGGAAUCAAGCCUCUGAACCCCGACGAC | |
| lowercase = | UUCUGGCGGUGUACAUCUGGCCUGCCUAGCCUGAUGAAG | |
| polyA tail | ACCCCUAAGAUCCGGCUGAUGCCUGGACCUGGACUGCUG | |
| (Amino acid | GCCAUGCCUACAACAGUGGAUGGCUGUGUGCGGACCCCU | |
| SEQ ID NO: | AGCCUGGUCAUCAACGAUCUGAUCUACGCCUACACCAGCA | |
| 724) | ACCUGAUCACCAGAGGCUGCCAGGAUAUCGGCAAGAGCUA | |
| | CCAGGUGCUGCAGAUCGGCAUCAUCACCGUGAACAGCGAU | |
| | CUGGUGCCCGACCUGAAUCCUCGGAUCAGCCACACCUUCA | |
| | ACAUCAACGACAACCGGAAGUCCUGUUCUCUGGCCCUGCU | |
| | GAACACCGACGUGUACCAGCUGUGUAGCACCCCUAAGGUG | |
| | GACGAGAGAAGCGACUAUGCCAGCAGCGGCAUCGAGGAUA | |
| | UCGUGCUGGACAUCGUGAACUACGACGGCAGCAUCAGCAC | |
| | CACCAGAUUCAAGAACAACAACAUCAGCUUCGACCAGCCU | |
| | UACGCCGCACUGUACCCUAGUGUUGGCCCUGGCAUCUAC | |
| | UACAAGGGCAAGAUCAUCUUCCUCGGCUACGGCGGCCUG | |
| | GAACACCCCAUCAAUGAGAACGCCAUCUGCAACACCACCG | |
| | GCUGUCCUGGCAAGACCCAGAGAGACUGCAAUCAGGCCAG | |
| | CCACUCUCCAUGGUUCAGCGACAGACGGAUGGUCAACUCU | |
| | AUCAUCGUGGUGGACAAGGGCCUGAACAGCGUGCCCAAG | |
| | CUGAAAGUGUGGACAAUCAGCAUGCGGCAGAACUACUGGG | |
| | GCAGCGAAGGCAGACUGCUGCUGCUGGGAAACAAGAUCUA | |
| | CAUCUACACCCGGUCCACCAGCUGGCACAGCAAACUGCAG | |
| | CUGGGAAUCAUCGACAUCACCGACUACAGCGACAUCCGGA | |
| | UCAAGUGGACCUGGCACAACGUGCUGAGCAGACCCGGCAA | |
| | CAAUGAGUGUCCUUGGGGCCACUCUUGCCCCGAUGGAUG | |
| | UAUCACCGGCGUGUACACCGACGCCUAUCCACUGAAUCCU | |
| | ACCGGCUCCAUCGUGUCCAGCGUGAUCCUGGACAGCCAG | |
| | AAAAGCAGAGUGAACCCCGUGAUCACAUACAGCACCGCCA | |
| | CCGAGAGAGUGAACGAGCUGGCCAUCAGAAACAAGACCCU | |
| | GAGCGCCGGCUACACCACCACAAGCUGCAUCACACACUAC | |
| | AACAAGGGCUACUGCUUCCACAUCGUCGAGAUCAACCACA | |
| | AGUCCCUGAACACCUUCCAGCCUAUGCUGUUCAAGACAGA | |
| | GAUCCCCAAGAGCUGCUCCUGAUGAGCUCGCUUUCUUGC | |
| | UGUCCAAUUUCUAUUAAAGGUUCCUUUGUUCCCUAAGUC | |
| | CAACUACUAAACUGGGGGAUAUUAUGAAGGGCCUUGAGC | |
| | AUCUGGAUUCUGCCUAAUAAAAAACAUUUAUUUUCAUUG | |
| | CAAaaaaaaaaaaaaaaaaaaaaaaaaaaaaaaaaaaaaaaaaaaaaaaaa | |
| | aaaaaaaaaaaaaaaaaaaaaaaaaaaaaaaa | |
| PIV3224_5UTR_ | AGGUGUCAGAGUUUAACUUGAAGACUAUUUCUAGGGAUA | 743 |
| 582/3UTR_ | AUAUCCCUGCCACCAUGGAAUACUGGAAGCACACCAACCA | |
| 2 80A_ | CGGCAAGGACGCCGGCAAUGAGCUGGAAACAAGCACAGCU | |
| modRNA | ACCCACGGCAACAAGCUGACCAACAAGAUCACCUACAUCC | |
| Underline = | UGUGGACCAUCACACUGGUGCUGCUGAGCAUCGUGUUCA | |
| 5′ cap; bold = | UCAUCGUGCUGACCAAUAGCAUCAAGAGCGACCUGAUUCA | |
| 5′UTR | GAGCGGCGUGAACACCCGGCUGCUGACAAUUCAGAGCCAC | |
| (5UTR_582) | GUGCAGAACUAUAUCCCCAUCAGCCUGACACAGCAGAUCA | |
| and 3′ UTR | GCGACCUGCGGAAGUUCAUCAGCGAGAUCACCAUCCGGAA | |
| (3UTR_2); | CGACAAUCAAGAGGUGCCACCACAGCGGAUCACCCACGAU | |
| italics = | GUGGGAAUCAAGCCUCUGAACCCCGACGACUUCUGGCGG | |
| KOZAK | UGUACAUCUGGCCUGCCUAGCCUGAUGAAGACCCCUAAGA | |
| sequence; | UCCGGCUGAUGCCUGGACCUGGACUGCUGGCCAUGCCUA | |
| lowercase = | CAACAGUGGAUGGCUGUGUGCGGACCCCUAGCCUGGUCA | |
| polyA tail | UCAACGAUCUGAUCUACGCCUACACCAGCAACCUGAUCAC | |
| (Amino acid | CAGAGGCUGCCAGGAUAUCGGCAAGAGCUACCAGGUGCU | |
| SEQ ID NO: | GCAGAUCGGCAUCAUCACCGUGAACAGCGAUCUGGUGCCC | |
| 725) | GACCUGAAUCCUCGGAUCAGCCACACCUUCAACAUCAACG | |
| | ACAACCGGAAGUCCUGUUCUCUGGCCCUGCUGAACACCGA | |
| | CGUGUACCAGCUGUGUAGCACCCCUAAGGUGGACGAGAG | |
| | AAGCGACUAUGCCAGCAGCGGCAUCGAGGAUAUCGUGCU | |
| | GGACAUCGUGAACUACGACGGCAGCAUCAGCACCACCAGA | |
| | UUCAAGAACAACAACAUCAGCUUCGACCAGCCUUACGCCG | |
| | CACUGUACCCUAGUGUUGGCCCUGGCAUCUACUACAAGGG | |
| | CAAGAUCAUCUUCCUCGGCUACGGCGGCCUGGAACACCCC | |
| | AUCAAUGAGAACGCCAUCUGCAACACCACCGGCUGUCCUG | |
| | GCAAGACCCAGAGAGACUGCAAUCAGGCCAGCCACUCUCC | |
| | AUGGUUCAGCGACAGACGGAUGGUCAACUCUAUCAUCGUG | |
| | GUGGACAAGGGCCUGAACAGCGUGCCCAAGCUGAAAGUG | |
| | UGGACAAUCAGCAUGCGGCAGAACUACUGGGGCAGCGAAG | |
| | GCAGACUGCUGCUGCUGGGAAACAAGAUCUACAUCUACAC | |
| | CCGGUCCACCAGCUGGCACAGCAAACUGCAGCUGGGAAUC | |
| | AUCGACAUCACCGACUACAGCGACAUCCGGAUCAAGUGGA | |
| | CCUGGCACAACGUGCUGAGCAGACCCGGCAACAAUGAGUG | |
| | UCCUUGGGGCCACUCUUGCCCCGAUGGAUGUAUCACCGG | |
| | CGUGUACACCGACGCCUAUCCACUGAAUCCUACCGGCUCC | |
| | AUCGUGUCCAGCGUGAUCCUGGACAGCCAGAAAAGCAGAG | |
| | UGAACCCCGUGAUCACAUACAGCACCGCCACCGAGAGAGU | |
| | GAACGAGCUGGCCAUCAGAAACAAGACCCUGAGCGCCGGC | |
| | UACACCACCACAAGCUGCAUCACACACUACAACAAGGGCUA | |
| | CUGCUUCCACAUCGUCGAGAUCAACCACAAGUCCCUGAAC | |
| | ACCUUCCAGCCUAUGCUGUUCAAGACAGAGAUCCCCAAGA | |
| | GCUGCUCCUGAUGAGCUCGCUUUCUUGCUGUCCAAUUUC | |
| | UAUUAAAGGUUCCUUUGUUCCCUAAGUCCAACUACUAAA | |
| | CUGGGGGAUAUUAUGAAGGGCCUUGAGCAUCUGGAUUCU | |
| | GCCUAAUAAAAAACAUUUAUUUUCAUUGCAAaaaaaaaaaaa | |
| | aaaaaaaaaaaaaaaaaaaaaaaaaaaaaaaaaaaaaaaaaaaaaaaaaaaa | |
| | aaaaaaaaaaaaaaaaa | |
| PIV3225_5UTR_ | AGGUGUCAGAGUUUAACUUGAAGACUAUUUCUAGGGAUA | 744 |
| 582/3UTR_ | AUAUCCCUGCCACCAUGGAAUACUGGAAGCACACCAACCA | |
| 2 80A_ | CGGCAAGGACGCCGGCAAUGAGCUGGAAACAAGCACAGCU | |
| modRNA | ACCCACGGCAACAAGCUGACCAACAAGAUCACCUACAUCC | |
| Underline = | UGUGGACCAUCACACUGGUGCUGCUGAGCAUCGUGUUCA | |
| 5′ cap; bold = | UCAUCGUGCUGACCAAUAGCAUCAAGAGCCGGAACGACAA | |
| 5′UTR | UCAAGAGGUGCCACCACAGCGGAUCACCCACGAUGUGGGA | |
| (5UTR_582) | AUCAAGCCUCUGAACCCCGACGACUUCUGGCGGUGUACAU | |
| and 3′ UTR | CUGGCCUGCCUAGCCUGAUGAAGACCCCUAAGAUCCGGCU | |
| (3UTR_2); | GAUGCCUGGACCUGGACUGCUGGCCAUGCCUACAACAGU | |
| italics = | GGAUGGCUGUGUGCGGACCCCUAGCCUGGUCAUCAACGA | |
| KOZAK | UCUGAUCUACGCCUACACCAGCAACCUGAUCACCAGAGGC | |
| sequence; | UGCCAGGAUAUCGGCAAGAGCUACCAGGUGCUGCAGAUC | |
| lowercase = | GGCAUCAUCACCGUGAACAGCGAUCUGGUGCCCGACCUGA | |
| polyA tail | AUCCUCGGAUCAGCCACACCUUCAACAUCAACGACAACCG | |
| (Amino acid | GAAGUCCUGUUCUCUGGCCCUGCUGAACACCGACGUGUA | |
| SEQ ID NO: | CCAGCUGUGUAGCACCCCUAAGGUGGACGAGAGAAGCGAC | |
| 726) | UAUGCCAGCAGCGGCAUCGAGGAUAUCGUGCUGGACAUC | |
| | GUGAACUACGACGGCAGCAUCAGCACCACCAGAUUCAAGA | |
| | ACAACAACAUCAGCUUCGACCAGCCUUACGCCGCACUGUA | |
| | CCCUAGUGUUGGCCCUGGCAUCUACUACAAGGGCAAGAUC | |
| | AUCUUCCUCGGCUACGGCGGCCUGGAACACCCCAUCAAUG | |
| | AGAACGCCAUCUGCAACACCACCGGCUGUCCUGGCAAGAC | |
| | CCAGAGAGACUGCAAUCAGGCCAGCCACUCUCCAUGGUUC | |
| | AGGGCCUGAACAGCGUGCCCAAGCUGAAAGUGUGGACAAU | |
| | AGCGACAGACGGAUGGUCAACUCUAUCAUCGUGGUGGACA | |
| | CAGCAUGCGGCAGAACUACUGGGGCAGCGAAGGCAGACU | |
| | GCUGCUGCUGGGAAACAAGAUCUACAUCUACACCCGGUCC | |
| | ACCAGCUGGCACAGCAAACUGCAGCUGGGAAUCAUCGACA | |
| | UCACCGACUACAGCGACAUCCGGAUCAAGUGGACCUGGCA | |
| | CAACGUGCUGAGCAGACCCGGCAACAAUGAGUGUCCUUGG | |
| | GGCCACUCUUGCCCCGAUGGAUGUAUCACCGGCGUGUAC | |
| | ACCGACGCCUAUCCACUGAAUCCUACCGGCUCCAUCGUGU | |
| | CCAGCGUGAUCCUGGACAGCCAGAAAAGCAGAGUGAACCC | |
| | CGUGAUCACAUACAGCACCGCCACCGAGAGAGUGAACGAG | |
| | CUGGCCAUCAGAAACAAGACCCUGAGCGCCGGCUACACCA | |
| | CCACAAGCUGCAUCACACACUACAACAAGGGCUACUGCUU | |
| | CCACAUCGUCGAGAUCAACCACAAGUCCCUGAACACCUUC | |
| | CAGCCUAUGCUGUUCAAGACAGAGAUCCCCAAGAGCUGCU | |
| | CCUGAUGAGCUCGCUUUCUUGCUGUCCAAUUUCUAUUAA | |
| | AGGUUCCUUUGUUCCCUAAGUCCAACUACUAAACUGGGG | |
| | GAUAUUAUGAAGGGCCUUGAGCAUCUGGAUUCUGCCUAA | |
| | UAAAAAACAUUUAUUUUCAUUGCAAaaaaaaaaaaaaaaaaaaa | |
| | aaaaaaaaaaaaaaaaaaaaaaaaaaaaaaaaaaaaaaaaaaaaaaaaaaaa | |
| | aaaaaaaaa | |

### Example 10. Immunogenicity Evaluation of modRNA-LNPs Combining Novel Compositions [PRL-Flu-Ms-2024-14]

This study evaluated the immunogenicity of novel LNP formulations compared with benchmark (ALC-315/DSPC/ALC-159/Cholesterol) LNP formulation using modRNA encoding HA/California, RSV preF and H1N1 A/California vaccine antigens.

The LNP formulations tested in this study are set forth in Table 55.

TABLE 55

| | Lipid Components | | | |
|---|---|---|---|---|
| | Ionizable | Polymer | Phospholipid | Sterol |
| LNP Formulation | (mol %) | (mol %) | (mol %) | (mol %) |
| Chol | ALC-315 | ALC-159 | DSPC | Cholesterol |
| [benchmark] | (47.5) | (1.8) | (10.0) | (40.7) |
| Sito/Chol = 6:4 | ALC-315 | ALC-159 | DSPC | Sito/Chol = 6/4 |
| | (47.5) | (1.8) | (10.0) | (24.4/16.3) |
| ESM-1 | ALC-315 | ALC-159 | ESM | Sitosterol |
| | (30.0) | (1.8) | (40.0) | (28.2) |
| ESM-2 | ALC-315 | ALC-159 | ESM | Sitosterol |
| | (35.0) | (1.8) | (35.0) | (28.2) |

Study details (Table 56): 10 week old female Balb/c mice were immunized on 0, 28 days with Cali/HA modRNA-LNP or on 0, 21 days with RSV A preF modRNA-LNP having the following LNP formulations described herein: (i) benchmark, (ii) sitosterol/cholesterol (6:4), or (iii) combined sphingomyelin/sitosterol. Each LNP formulation was then evaluated for immunogenicity in the mice using neutralization assays that are well-known in the art. All modRNA-LNPs were stored at 5° C. before administration.

TABLE 56

| | | | | Dose | Vax | Bleed |
|---|---|---|---|---|---|---|
| Grp | Sterol Description | | Antigen | Vol/Route | day | day |
| 1 | Saline | | modRSV | | | |
| 2 | Cholesterol [benchmark] | 0.2 | | 50 μL/IM | 0, 21 | 21, 35 |
| 3 | Sito/Chol = 6:4 | 0.2 | | | | |
| 4 | ESM-1 (Cat/ESM = 30/40) | 0.2 | | | | |
| | [sitosterol] | | | | | |
| 5 | ESM-2 (Cat/ESM = 35/35) | 0.2 | | | | |
| | [sitosterol] | | | | | |
| 8 | Saline | N/A | mod HA/Cali | | | |
| 9 | Cholesterol [benchmark] | 0.2 | | 50 μL/IM | 0, 28 | 21, 42 |
| 10 | Sito/Chol = 6:4 | 0.2 | | | | |
| 11 | ESM-1 (Cat/ESM = 30/40) | 0.2 | | | | |
| | [sitosterol] | | | | | |
| 12 | ESM-2 (Cat/ESM = 35/35) | 0.2 | | | | |
| | [sitosterol] | | | | | |
| | Saline | N/A | mod H1N1 A/Cali | | | |
| | Cholesterol [benchmark] | 0.2 | | 50 μL/IM | 0, 28 | 21, 42 |
| | Sito/Chol = 6:4 | 0.2 | | | | |
| | ESM-1 (Cat/ESM = 30/40) | 0.2 | | | | |
| | [sitosterol] | | | | | |
| | ESM-2 (Cat/ESM = 35/35) | 0.2 | | | | |
| | [sitosterol] | | | | | |

### Results

TABLE 57
Formulation Analytical Data

| | | | | | | | | DP |
|---|---|---|---|---|---|---|---|---|
| | Sterol | | | | DP size | | DP conc | HEK293T |
| Grp | Description | Antigen | % FA | % EE | (d · nM) | PDI | (μg/ml) | EC50 |
| 2 | Cholesterol [benchmark] | modRSV | 93 | 97 | 65 | 0.04 | 137 | 31.7 |
| 3 | Sito/Chol = 6:4 | | 90 | 93 | 93 | 0.05 | 131 | 0.694 |
| 4 | ESM-1 (Cat/ESM = 30/40) | | 93 | 90 | 99 | 0.06 | 113 | 1.32 |
| 5 | ESM-2 (Cat/ESM = 35/35) | | 93 | 89 | 103 | 0.05 | 116 | 0.645 |
| 9 | Cholesterol [benchmark] | mod | 84 | 98 | 64 | 0.03 | 138 | 2.7 |
| 10 | Sito/Chol = 6:4 | HA/Cali | 82 | 95 | 90 | 0.04 | 141 | 0.091 |
| 11 | ESM-1 (Cat/ESM = 30/40) | | 86 | 92 | 99 | 0.08 | 127 | 0.18 |
| 12 | ESM-2 (Cat/ESM = 35/35) | | 86 | 91 | 101 | 0.05 | 126 | 0.086 |

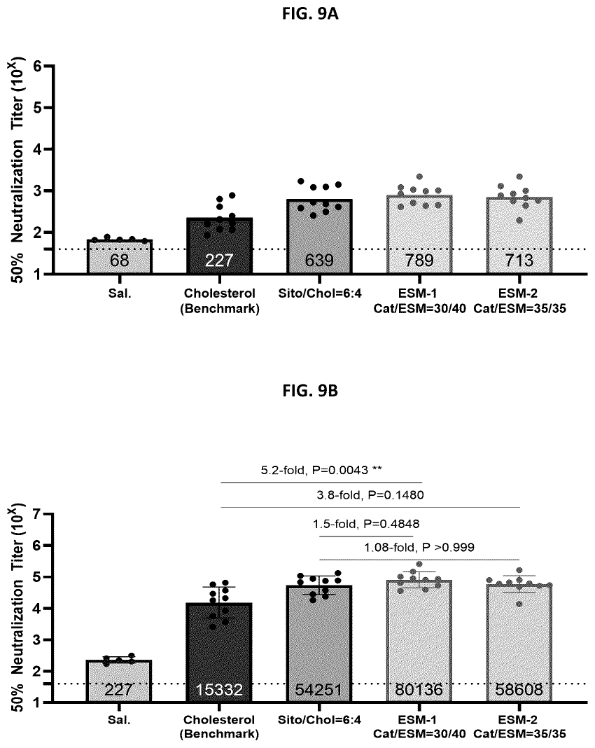

ESM formulations increased immunogenicity of Flu HA/Cali up to 5.2-fold compared to benchmark (cholesterol) and comparable or slightly higher titers than the sitosterol/cholesterol formulation (Sito/Chol=6:4) as shown in FIG. 9A (3 weeks PD1) and FIG. 9B (2 weeks PD2).

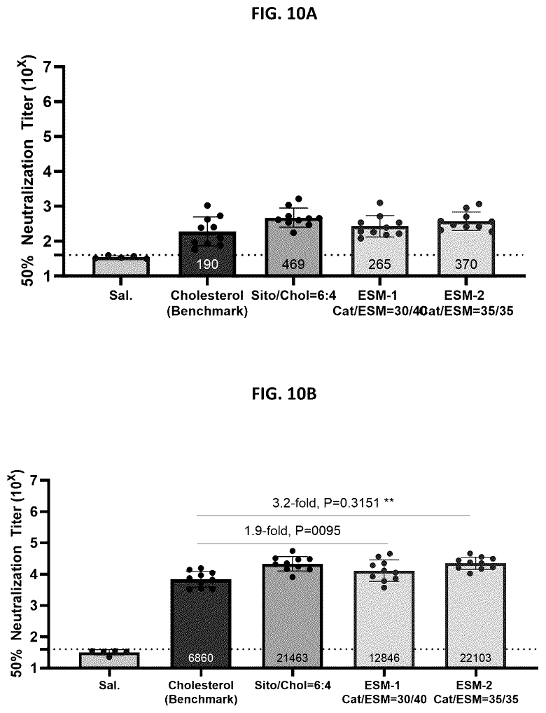

ESM formulations increased immunogenicity of RSV A preF up to 3.2-fold compared to benchmark (cholesterol) and comparable or slightly higher titers than the sitosterol/cholesterol formulation (Sito/Chol=6:4) as shown in FIG. 10A (3 weeks PD1) and FIG. 10B (2 weeks PD2).

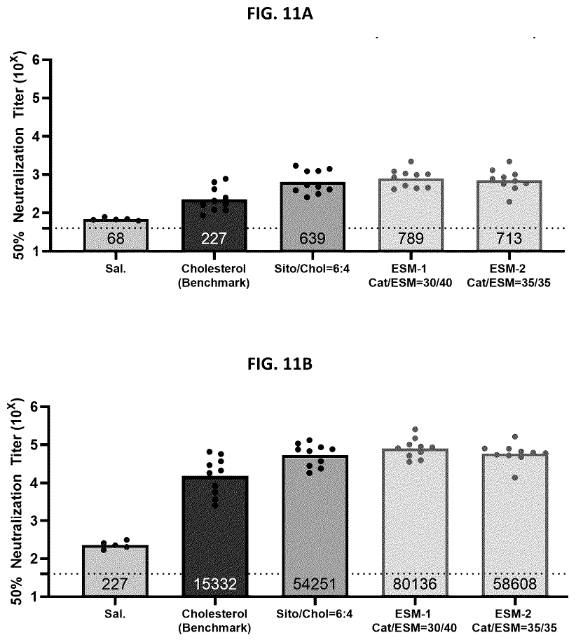

H1N1 A/California modRNA formulations were tested in mice as described in Table 56. Results show that ESM formulations increased immunogenicity of H1N1 A/California compared to benchmark (cholesterol) and comparable or slightly higher titers than the sitosterol/cholesterol formulation (Sito/Chol=6:4) as shown in FIG. 11A (3 weeks PD1) and FIG. 11B (2 weeks PD2).

### Example 11—Lyophilization of Mod RSVpreF RNA-Encapsulated LNP with Sodium Oleate

The formulation below uses water as the reconstitution diluent and was assessed for Size, PDI, EE, IVE, and immunogenicity. The results shown in Table 59 demonstrate that lyophilization of modRNA LNP formulations is feasible.

TABLE 58

| Attributes | Lyo presentation |
|---|---|
| DP storage temperature (° C.) | 5 |
| Vial size | 2 mL |
| DP conc. (mg/mL) | 0.1 (modRSV) |
| DP fill volume (mL) | 0.5 |
| DP matrix | 10 mM Tris in 300 mM Sucrose, and |
| | sodium oleate (0 ug/mL [O:R 0], 200 |
| | ug/mL [O:R 2], 400 ug/mL [O:R 4], |
| | and 800 ug/mL [O:R 8]), pH 7.4 |
| Diluent/Reconstituent | Water |
| Recon. Vol. (mL) | 0.47 |
| Post dilution/recon. DP | mRNA RSV w/0.47 mL recon - |
| concentration (mg/mL) | 100 ug/mL |

TABLE 59
Analytical Data: modRSVpreF mRNA-
encapsulated LNP w/sodium oleate

| | | | | IVE |
|---|---|---|---|---|
| Formulation | | FA | Encap- | (% +/EC50) |
| [Oleate:RNA] | Size/PDI | (Main/LMS) | sulation | (256 ng) |
| modRSV Control | 76/0.14 | 91/NMT 3% | 94 | 74/43 |
| Liquid [O:R 0] | | | | |
| modRSV Control | 85/0.14 | 92/NMT 3% | 79 | 75/34 |
| Lyo [O:R 0] | | | | |
| modRSV w/200 | 77/0.12 | 91/NMT 3% | 90 | 71/56 |
| ug/mL sodium | | | | |
| oleate | | | | |
| Liquid [O:R 2] | | | | |
| modRSV w/200 | 83/0.15 | 92/NMT 3% | 59 | 67/76 |
| ug/mL sodium | | | | |
| oleate | | | | |
| Lyo [O:R 2] | | | | |
| modRSV w/400 | 74/0.06 | 92/NMT 3% | 84 | 72/44 |
| ug/mL sodium | | | | |
| oleate | | | | |
| Liquid [O:R 4] | | | | |
| modRSV w/400 | 78/0.11 | 90/NMT 3% | 50 | 66/74 |
| ug/mL sodium | | | | |
| oleate | | | | |
| Lyo [O:R 4] | | | | |
| modRSV w/800 | 78/0.08 | 91/NMT 3% | 82 | 80/32 |
| ug/mL sodium | | | | |
| oleate | | | | |
| Liquid [O:R 8] | | | | |
| modRSV w/800 | 79/0.10 | 89/NMT 3% | 70 | 75/50 |
| ug/mL sodium | | | | |
| oleate | | | | |
| Lyo [O:R 8] | | | | |

In Tables 58 and 59, the term “Liquid” indicates the respective formulation prior to lyophilization, e.g., “pre-lyophilization”; the term “Lyo” indicates that the respective formulation is lyophilized.

### Example 12—Sodium Oleate Improves Liquid Stability of Mod RSVpreF RNA-Encapsulated LNP

Samples of RSVmodRNA (preF A) in a benchmark LNP as set forth below were used: 0.1 mg/mL modRSV A drug product in 10 mM Tris and 300 mM Sucrose having either
no sodium oleate (control, [O:R 0])
0.4 mg/mL sodium oleate [O:R 4]
0.8 mg/mL sodium oleate [O:R 8]

The resulting samples were staged as never frozen (NF) and 1× freeze/thaw (F/T) at 5° C. over a 6 month period of time. LNPs comprising a high quality ALC-0315 lipid having low starting glyoxal and aldehyde impurities was used as a comparator, which is designated “HQ Lipid” or “Lower Impurity Lipid” tested herein at an O:R of 0.

As shown in Tables 60 and 61 sodium oleate improves the overall stability, reduces adduct formation, and maintains expression at 5° C. for benchmark LNPs that were never frozen (liquid) or underwent freeze/thaw.

TABLE 60
Refrigerated Stability (5° C.) for Never Frozen Formulations

| | | | Integrity | | | | | | |
|---|---|---|---|---|---|---|---|---|---|
| RSVmodRNA- | | | Main | Late | Protein | | | | |
| LNP Sample | | EE | Peak to | Migration | Expression | IVE | Diam | | % |
| [Oleate:RNA] | TP/Temp | (%) | Total (%) | Peaks (%) | (%) | (EC50) | (nm) | PDI | Adducts |
| O:R 0 | T = 0, 5 C. | 98 | 93 | 3 | 70 | 79 | 71 | 0.08 | 3 |
| O:R 4 | T = 0, 5 C. | 89 | 91 | 3 | 73 | 72 | 74 | 0.1 | 3 |
| O:R 8 | T = 0, 5 C. | 90 | 91 | 3 | 79 | 49 | 72 | 0.05 | 2 |
| Low Impurity | T = 0, 5 C. | 97 | 93 | 3 | 69 | 86 | 75 | 0.08 | 2 |
| Lipid | | | | | | | | | |
| O:R 0 | 3 month, | 95 | 85 | 5 | 82 | 61 | 68 | 0.09 | 22 |
| | 5° C. | | | | | | | | |
| O:R 4 | 3 month, | 87 | 84 | 3 | 84 | 61 | 74 | 0.11 | 10 |
| | 5° C. | | | | | | | | |
| O:R 8 | 3 month, | 88 | 82 | 3 | 91 | 41 | 72 | 0.04 | 4 |
| | 5° C. | | | | | | | | |
| Low Impurity | 3 month, | 95 | 86 | 4 | 89 | 48 | 75 | 0.1 | 8 |
| Lipid | 5° C. | | | | | | | | |
| O:R 0 | 4.5 month, | 95 | 82 | 9 | 74 | 57 | 69 | 0.08 | 25 |
| | 5° C. | | | | | | | | |
| O:R 4 | 4.5 month, | 88 | 81 | 5 | 81 | 36 | 72 | 0.07 | 16 |
| | 5° C. | | | | | | | | |
| O:R 8 | 4.5 month, | 89 | 81 | 3 | 80 | 35 | 72 | 0.03 | 5 |
| | 5° C. | | | | | | | | |
| Low Impurity | 4.5 month, | 94 | 85 | 7 | 76 | 44 | 74 | 0.08 | 19 |
| Lipid | 5° C. | | | | | | | | |
| JO:R 0 | 6 month, | 97 | 83 | 7 | 91 | 31 | 69 | 0.08 | 26 |
| | 5° C. | | | | | | | | |
| O:R 4 | 6 month, | 89 | 80 | 4 | 95 | 20 | 72 | 0.05 | 24 |
| | 5° C. | | | | | | | | |
| O:R 8 | 6 month, | 90 | 80 | 3 | 94 | 21 | 72 | 0.05 | 7 |
| | 5° C. | | | | | | | | |
| Low Impurity | 6 month, | 97 | 81 | 9 | 91 | 28 | 75 | 0.09 | 22 |
| Lipid | 5° C. | | | | | | | | |

Table 60 shows stability results of never frozen modRSV A DP with sodium oleate, namely, that the NF DP with sodium oleate was stable up to 6 months at 5° C., exhibited less adduct formation and higher T=0 expression (IVE).

TABLE 61
Post Freeze/Thaw Stability (5° C.)

| | | | Integrity | Late | | | | | |
|---|---|---|---|---|---|---|---|---|---|
| | | | Main | Migration | Protein | | | | |
| | | | Peak to | Peaks | Expression | EC50 | Diam | | |
| Sample | TP/Temp | EE(%) | Total (%) | (%) | (%) | (IVE) | (nm) | PDI | % Adducts |
| O:R 0 | Freeze/Thaw, | 97 | 93 | 3 | 70 | 83 | 70 | 0.09 | 1 |
| | T = 0 | | | | | | | | |
| O:R 4 | Freeze/Thaw, | 89 | 93 | 3 | 74 | 67 | 79 | 0.13 | 1 |
| | T = 0 | | | | | | | | |
| O:R 8 | Freeze/Thaw, | 90 | 93 | 3 | 85 | 47 | 73 | 0.06 | 1 |
| | T = 0 | | | | | | | | |
| O:R 0 | Freeze/Thaw | 94 | 85 | 4 | 69 | 105 | 84 | 0.26 | 15 |
| | +3 month, | | | | | | | | |
| | 5° C. | | | | | | | | |
| O:R 4 | Freeze/Thaw | 88 | 83 | 3 | 84 | 47 | 75 | 0.09 | 10 |
| | +3 month, | | | | | | | | |
| | 5° C. | | | | | | | | |
| O:R 8 | Freeze/Thaw | 90 | 81 | 3 | 88 | 40 | 73 | 0.05 | 4 |
| | +3 month, | | | | | | | | |
| | 5° C. | | | | | | | | |
| O:R 0 | Freeze/Thaw | 92 | 84 | 7 | 65 | 120 | 98 | 0.39 | 22 |
| | +4.5 month, | | | | | | | | |
| | 5° C. | | | | | | | | |
| O:R 4 | Freeze/Thaw | 86 | 82 | 4 | 79 | 34 | 73 | 0.08 | 12 |
| | +4.5 month, | | | | | | | | |
| | 5° C. | | | | | | | | |
| O:R 8 | Freeze/Thaw | 89 | 82 | 3 | 89 | 22 | 73 | 0.04 | 6 |
| | +4.5 month, | | | | | | | | |
| | 5° C. | | | | | | | | |
| O:R 0 | Freeze/Thaw | 88 | 78 | 11 | 71 | 109 | 109 | 0.44 | 32 |
| | +6 month, | | | | | | | | |
| | 5° C. | | | | | | | | |
| O:R 4 | Freeze/Thaw | 89 | 79 | 3 | 94 | \|25 | 75 | 0.11 | 18 |
| | +6 month, | | | | | | | | |
| | 5° C. | | | | | | | | |
| O:R 8 | Freeze/Thaw | 90 | 80 | 3 | 95 | 23 | 72 | 0.07 | 7 |
| | +6 month, | | | | | | | | |
| | 5° C. | | | | | | | | |

Table 61 shows stability results for frozen modRSV A DP which was thawed and stored at 5 C up to 6 months. The results show that sodium oleate improves the overall stability, reduces adduct formation and maintains expression at 5° C. for benchmark LNPs that have undergone freeze/thaw.

### Example 13—Alternative Cationic Lipids in LNP Formulations Comprising Oleate

### Samples Prepared:

0.1 mg/mL modRSV A drug product in 10 mM Tris and 300 mM Sucrose
LNP comprising:
   ATX Cationic Lipid, b-sitosterol/Chol (6:4), DSPC, and ALC-0159 [O:R 0],
   ATX Cationic Lipid, b-sitosterol/Chol (6:4), DSPC, and ALC-0159 w/ oleic acid [O:R 8],
   A9 Cationic Lipid, b-sitosterol/Chol (6:4), DSPC, and ALC-0159 [O:R 0], or
   A9 Cationic Lipid, b-sitosterol/Chol (6:4), DSPC, and ALC-0159 w/ oleic acid [O:R 8].

Samples included ATX (Arcturus) and A9 (Acuitas) cationic lipids which were tested as 5° C. (never frozen) and −80° C. (1×F/T to 5° C.). LNPs made with b-sitosterol typically undergo higher particle size changes post freeze/thaw which is mitigated when the formulations include sodium oleate/oleic acid (oleate). An additional benefit of including oleate is that lipid-RNA adducts are suppressed on liquid storage (see Table 62). Accordingly, the benefits of adding oleate to the DP are maintained regardless of the cationic lipid used to form the LNP.

TABLE 62
Analytical Data

| | | | Integrity | | |
|---|---|---|---|---|---|
| Sample | Temp | EE | (Main/LMS) | Size/PDI | % Adduct |
| ATX | 5° C. | >97% | 88%/4% | 106/0.08 | 22 |
| ATX | −80° C. | 73% | 88%/4% | 163/0.20 | N/T |
| ATX | 5° C. | 79% | 88%/3% | 78/0.10 | 8 |
| w/oleate | | | | | |
| ATX | −80° C. | 79% | 89%/3% | 77/0.11 | N/T |
| w/oleate | | | | | |
| A9 | 5° C. | 94% | 83%/9% | 108/0.07 | 13 |
| A9 | −80° C. | 90% | 86%/7% | 168/0.42 | N/T |
| A9 | 5° C. | 86% | 90%/3% | 79/0.06 | 5 |
| w/oleate | | | | | |
| A9 | −80° C. | 86% | 89%/3% | 79/0.06 | N/T |
| w/oleate | | | | | |

N/T = not tested

### Example 14—PRL-RSV-Ms-2024-08: Evaluation of RSV preF modRNA Encapsulated with LNPs Comprising PF-07929032 (9032)

In this in vivo study, mice were dosed according to the details set forth in Table 63 and results 2 weeks post dose 2 (2WPD2) shown in Table 64.

TABLE 63
In vivo study

| | Cationic Lipid/LNP | | Dose | | |
|---|---|---|---|---|---|
| | formulation +/− | Dose | Volume/ | Vax | Bleed |
| Mice | sodium oleate (SO) [O:R] | (μg) | Route | (Day) | (Day) |
| 10 | Saline | — | 50 μL/IM | 0.21 | 21, 35 |
| 10 | ALC-0315 [O:R 0]* | 0.2 | 50 μL/IM | 0.21 | 21, 35 |
| 10 | PFE-07929032 [O:R 0]* | 0.2 | 50 μL/IM | 0.21 | 21, 35 |
| 10 | PF-07929032 high impurity | 0.2 | 50 μL/IM | 0.21 | 21, 35 |
| | batch [O:R 0]* | | | | |
| 10 | ALC-0315/Sito/SO | 0.2 | 50 μL/IM | 0.21 | 21, 35 |
| | [O:R 8]** | | | | |
| 10 | PF-07929032/Sito/SO | 0.2 | 50 μL/IM | 0.21 | 21, 35 |
| | [O:R 8]** | | | | |

*0.24 mg/mL modRSV preF A drug product in 10 mM Tris and 300 mM Sucrose with LNP: Cationic Lipid (ALC-0315 or PF-07929032), Cholesterol, DSPC, and ALC-0159, (O:R 0)
**0.24 mg/mL modRSV preF A drug product in 10 mM Tris and 300 mM Sucrose with LNP: Cationic lipid (ALC-0315 or PF-07929032), b-sitosterol/Chol (6:4 mol ratio), DSPC, and ALC-0159, w/2 mg/mL sodium oleate (O:R 8) added via spike process.

TABLE 64
2WPD2 50% Boost Neutralization Titers for RSV A and RSV B

| Sample | RSV | RSV |
|---|---|---|
| (oleate:RNA ratio) | A | B |
| ALC-0315* (O:R 0) | 10158 | 4205 |
| 9032* (O:R 0) | 23200 | 6751 |
| 9032 High Imp* | 33699 | 11995 |
| (O:R 0) | | |
| ALC-0315/sito/SO** | 60190 | 24400 |
| (O:R 8) | | |
| 9032/sito/SO** | 37765 | 9666 |
| (O:R 8) | | |

The results show that ALC-0315 was more immunogenic than PFE-07929032 (9032) for both RSV A and RSV B response in the formulations described herein. The impurity level of PFE-07929032 had no impact on RSV immunogenicity. There was ˜6-fold increase in response from ALC-0315 LNP formulation with the incorporation of β-sitosterol and sodium oleate.

### Example 15—Alternative Fatty Acids in LNP Formulations

### Samples Prepared:

0.1 mg/mL modRSV A drug product in 10 mM Tris and 300 mM Sucrose
LNP comprising:
   ALC-0315, b-sitosterol/Chol (6:4), DSPC, and ALC-0159 w/ arachidonic acid
   ALC-0315, b-sitosterol/Chol (6:4), DSPC, and ALC-0159 w/ eruric acid
   ALC-0315, b-sitosterol/Chol (6:4), DSPC, and ALC-0159 w/ linolenic
   ALC-0315, b-sitosterol/Chol (6:4), DSPC, and ALC-0159 w/ ricinoleic acid
   ALC-0315, b-sitosterol/Chol (6:4), DSPC, and ALC-0159 w/ palmitoleic acid
   ALC-0315, b-sitosterol/Chol (6:4), DSPC, and ALC-0159 w/ linoleic acid

Resulting samples [all O:R 8] were tested as 5 C (never frozen) and −80 C (1×F/T). LNPs made with b-sitosterol typically undergo higher particle size changes post freeze/thaw which is mitigated when the formulations include mono-, di-, and poly-unsaturated fatty acids. An additional benefit of including fatty acids is that lipid-RNA adducts are suppressed on liquid storage (see Table 65).

TABLE 65
Analytical Data [O:R 8]**

| Sample LNP | | Integrity | | |
|---|---|---|---|---|
| Fatty acid | EE | (Main/LMS) | Size/PDI | % Adduct |
| Arachidonic | 85% | 91%/NMT 3% | 92/0.03 | 1 |
| (liquid) | | | | |
| Arachidonic | 85% | 91%/NMT 3% | 91/0.02 | N/T |
| (freeze/thaw) | | | | |
| Eruric | 90% | 89%/4% | 82/0.05 | 4 (benefit may be |
| (liquid) | | | | seen over time) |
| Eruric | 89% | 90%/3% | 83/0.07 | N/T |
| (freeze/thaw) | | | | |
| Linolenic | 87% | 90%/NMT 3% | 90/0.04 | 2 |
| (liquid) | | | | |
| Linolenic | 88% | 92%/NMT 3% | 90/0.05 | N/T |
| (freeze/thaw) | | | | |
| Ricinoleic | 54% | 89%/NMT 3% | 113/0.10 | 2 |
| (liquid) | | | | |
| Ricinoleic | 46% | 91%/NMT 3% | 113/0.07 | N/T |
| (freeze/thaw) | | | | |
| Palmitoleic | 89% | 89%/NMT 3% | 88/0.04 | 1 |
| (liquid) | | | | |
| Palmitoleic | 89% | 91%/NMT 3% | 89/0.03 | N/T |
| (freeze/thaw) | | | | |
| Linoleic | 94% | 92%/NMT 3% | 87/0.04 | 1 |
| (liquid) | | | | |
| Linoleic | 71% | 91%/NMT 3% | 117/0.13 | N/T |
| (freeze/thaw) | | | | |
| Non-FA | 93% | 91%/NMT 3% | 80/0.16 | 3 |
| Comparator | | | | |
| (liquid) | | | | |
| Non-FA | N/T | N/T | 104/0.20 | 2 |
| Comparator | | | | |
| (freeze/thaw) | | | | |

N/T = not tested

LNP formulations containing fatty acids or derivatives or salts thereof result in better maintained particle size after completion of the respective freezing and thawing process with the exception of linoleic acid compared to the non-fatty acid containing comparator. LNP formulations containing fatty acids or derivatives or salts thereof result in better maintained polydispersity after completion of the respective freezing and thawing process with the exception of linoleic acid compared to the non-fatty acid containing comparator. LNP formulations containing fatty acids or derivatives or salts thereof result in better maintained encapsulation after completion of the respective freezing and thawing process with the exception of linoleic acid. It is expected that similar benefits would be found over time when using unsaturated mono-, di-, and polyunsaturated fatty acids or derivatives or salts thereof.

### Example 16: Combination and Formulation Strategies of RSV, HMPV, and PIV3 Vaccine Components

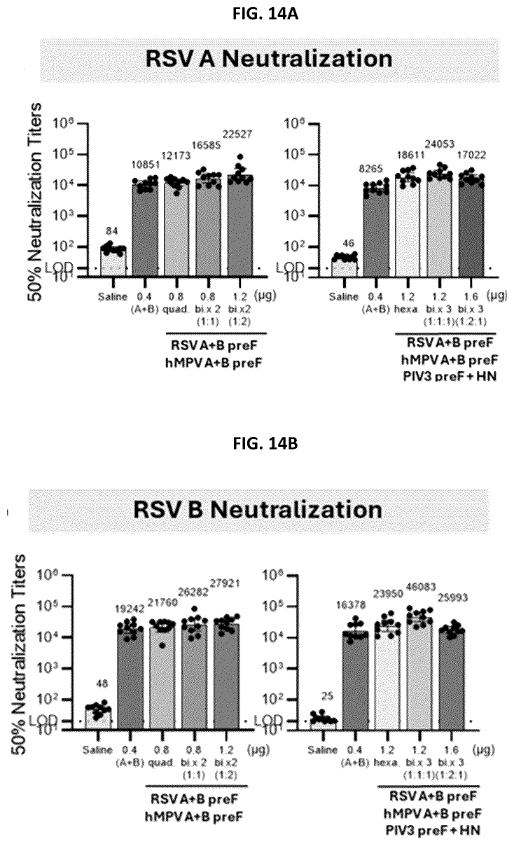

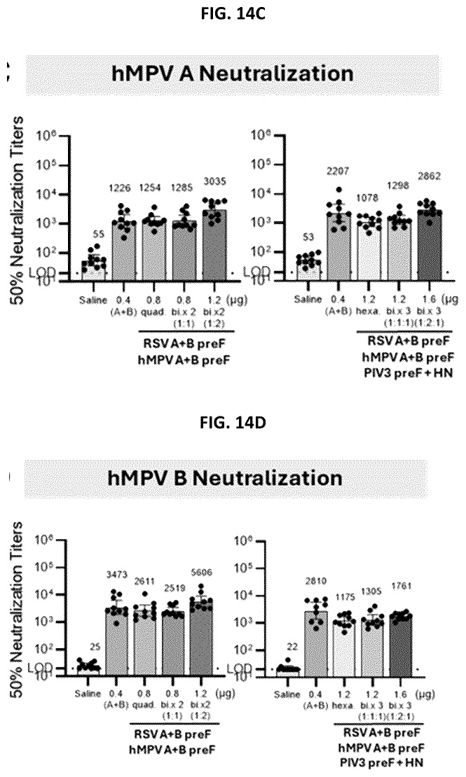

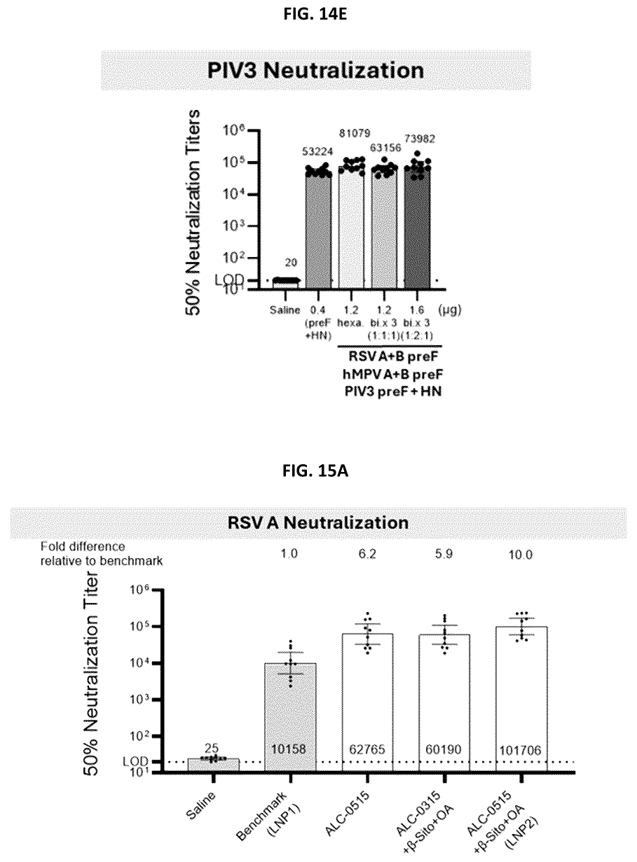

This Example describes the combination of up to six modRNA components in one drug product formulation. Immunogenicity of different modRNA combination formulation approaches of modRNA RSVpreF A+B, hMPVpreF A+B, and PIV3 preF+HN components was evaluated using the benchmark LNP (LNP1, described in Example 2). In the modRNA combinations, both a quadrivalent formulation, consisting of equal weight ratios of RSVpreF A and B and hMPVpreF A and B modRNAs, and a hexavalent combination, consisting of equal weight ratios of RSVpreF A and B, hMPVpreF A and B, PIV3preF and PIV3 HN, were compared to RSV, hMPV or PIV3 alone groups. Mice were immunized with two doses of co-formulated bivalent RSV A+B at 0.4 μg dose (1:1 ratio), co-formulated quadrivalent RSVpreF A+B and hMPVpreF A+B at 0.8 μg dose (1:1:1:1 ratio, referred to as “quad.” in FIG. 14A-14D), a 1:1 mixture of co-formulated bivalent RSVpreF A+B with co-formulated bivalent hMPVpreF A+B at 0.8 μg dose (1:1 and 1:1 ratio, referred to as “bi. x 2 (1:1)” in FIG. 14A-14D), or a 1:2 mixture of co-formulated bivalent RSVpreF A+B with two-fold more co-formulated bivalent hMPVpreF A+B at 1.2 μg dose (1:1 and 2:2 ratio, referred to as “bi. x 2 (1:2)” in FIG. 14A-14D). RSV neutralization titers of the two quadrivalent groups were comparable with the bivalent RSV alone group (FIGS. 14A and 14B). Increasing the total amount of hMPVpreF by two-fold did not negatively impact the RSV A or RSV B titers.

Using a similar experimental design, combinations with PIV3 preF and HN were also tested in a separate study as follows: (1:1:1:1:1:1 RNA weight ratio, referred to as “hexa.” in FIG. 14A-14E), a 1:1:1 mixture of co-formulated bivalent RSVpreF A+B, bivalent hMPVpreF A+B, and bivalent PIV3preF and PIV3HN (1:1, 1:1, and 1:1 RNA weight ratio, referred as “bi. x 3 (1:1:1)” in FIG. 14A-14E), or a 1:2:1 mixture of co-formulated bivalent RSVpreF A+B, 2-fold bivalent hMPVpreF A+B, bivalent PIV3preF and PIV3HN (1:1, 2:2, and 1:1 ratio, referred to as “bi. x 3 (1:2:1)” in FIG. 14A-14E). The neutralization titers of the hexavalent combination groups induced statistically comparable neutralization titers, but both groups trended higher compared with the RSVpreF A+B group, which could potentially be attributed to the adjuvant effect of higher total dose of LNP-formulated modRNA in the hexavalent group. Increasing the hMPV components in the hexavalent combination (“bi. x 3 (1:2:1)” led to lower trend of RSV A neutralizing titer (FIG. 14A). RSV B neutralization results also demonstrated similar trends across the groups (FIG. 14B). The hMPV A and B neutralization data is plotted and presented in the identical fashion with RSV A neutralization results (FIGS. 14C and 14D). At Day 35, the quadrivalent combination groups demonstrated comparable neutralization titers compared with the bivalent hMPV alone group.

The hexavalent combination groups induced comparable neutralizing titers, but both groups trended lower compared with the bivalent hMPV A+B group. Increasing the hMPV component by 2-fold (“bi. x2 (1:2)” or “bi. x3 (1:2:1)”) increased the hMPV A neutralization titers by approximately two-fold in both the quadrivalent and hexavalent groups, while this trend is less evident in the hMPV B neutralization titer in the hexavalent group. Finally, the PIV3 neutralization titers of the hexavalent combination groups were comparable with the bivalent PIV3 preF+HN group (FIG. 14E). Overall, these data demonstrated the feasibility of combining multiple modRNA-based antigens, including both the quadrivalent and hexavalent formulations, without impacting the immunogenicity of each component.

### Example 17: Selection of Novel LNP Formulations

Novel LNP formulations were developed by replacing or incorporating new LNP components such as ionizable cationic lipids or cholesterol analogs with excipients such as fatty acids or salts thereof that may improve immunogenicity. Ionizable cationic lipid ALC-0515 was found to improve immunogenicity when used as a replacement for ALC-0315. Additionally, a naturally-occurring cholesterol analog called β-sitosterol, in combination with cholesterol at a specific ratio, was shown to enhance immunogenicity with oleic acid, which is a fatty acid excipient that has the potential to stabilize the formulation as previously described in PCT Internation Application No. PCT/IB2025/052355 filed Mar. 4, 2025, which is hereby incorporated by reference herein in its entirety.

Using RSV A preF encoded modRNA as a model, the following LNP formulations were tested:
i. the benchmark formulation (ALC-0315/ALC-0159/DSPC/cholesterol, referred to as LNP1),
ii. ALC-0515 replacement formulation (ALC-0515/ALC-0159/DSPC/cholesterol),
iii. β-sitosterol:cholesterol with oleic acid mixture (ALC-0315/ALC-0159/DSPC/β-sitosterol:cholesterol/oleic acid), and
iv. a combination of both ALC-0515 replacement and β-sitosterol:cholesterol with oleic acid mixture, wherein the molar ratio of β-sitosterol:cholesterol is 6:4. (ALC-0515/ALC-0159/DSPC/cholesterol:β-sitosterol/oleic acid, herein referred to as LNP2).
   The lipid excipients in the LNP formulations are further described in Table 66.

TABLE 66
Lipid Excipients in the LNP Formulations

| | | | Physical | |
|---|---|---|---|---|
| | Molecular | | State and | |
| | Weight | Molecular | Storage | Chemical Name (Synonyms) and |
| Lipid | [Da] | Formula | Condition | Structure |
| ALC- 0315^{a} | 766 | C48H95N O5 | Liquid (oil) | ((4-hydroxybutyl)azanediyl)bis(hexane-6,1- diyl)bis(2-hexyldecanoate) 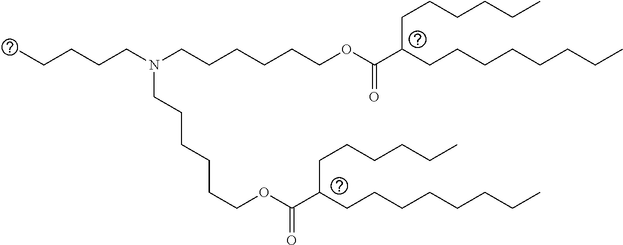 [SMILES: CCCCCCCCC(CCCCCC)C(=O)OCCCCCCN(CCCCC)CCCCCCOC(=O)C(CCCCCC)CCCCCCCC] |
| ALC- 0159^{b} | 2100- 2700 | (C2H4O)n C31H63N O2 | Solid −20° C. | 2-[(polyethylene glycol)-2000]- N,N-ditetradecylacetamide 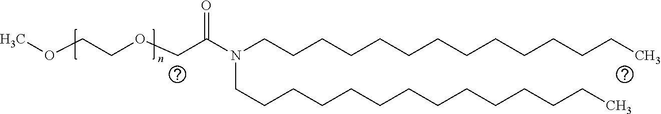 |
| DSPC^{c} | 790 | C44H88N O8P | Solid −20° C. | 1,2-distearoyl-sn-glycero-3-phosphocholine 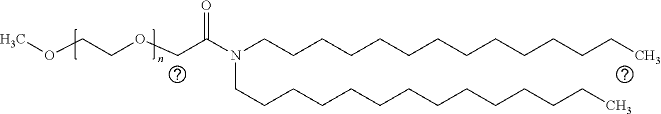 |
| Cholesterol^{d} | 387 | C27H46O | Solid −20° C. | 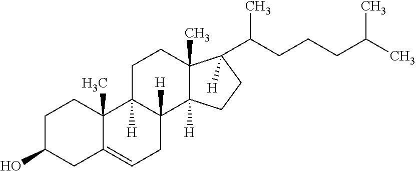 |
| ALC- 0515^{e} | 948.6 | C59H117 N3O5 | Liquid (oil) | bis(2-hexyldecyl) 6,6′-({2-[methyl(4- octanamidobutyl)amino]ethyl}azanediyl) dihexanoate 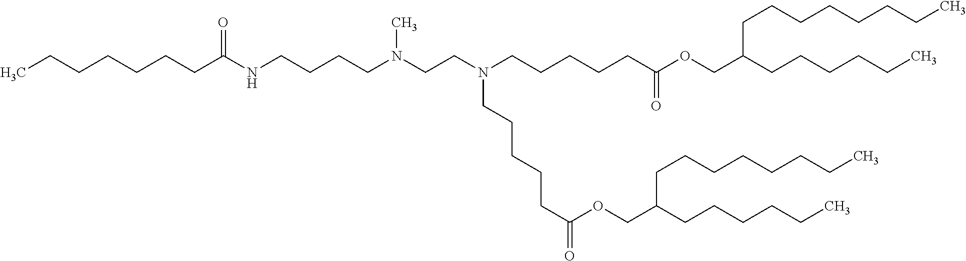 [SMILES: CCCCCCCCC(CCCCCC)COC(=O)CCCCCN(CCCCCC(=O)OCC(CCCCCC)CCCCCCCC)CCN(C)CCCCNC(=O)CCCCCCC] |
| beta- Sitosterol^{f} | 414.7 | C29H50O | Solid | (−)-beta-Sitosterol; 22,23- Dihydrostigmasterol 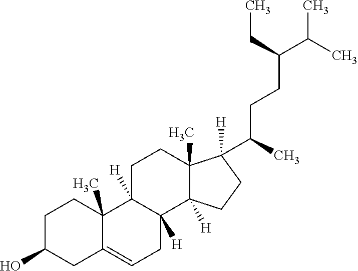 [SMILES: CC[C@H](CC[C@@H](C)[C@H]1CC[C@H]2[C@@H]3CC=C4C[C@@H](O)CC[C@]4(C)[C@H]3CC[C@]12C)C(C)C] |
| Oleic acid^{g} | 282.47 | C18H34O2 | Liquid (Oil) | (9Z)-octadec-9-enoic acid; (Z)-9- Octadecenoic acid 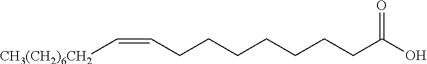 |

^{a}. CAS Number 2036272-55-4
^{b}. CAS Number 1849616-42-7
^{c}. CAS Number 816-94-4
^{d}. CAS Number 57-88-5
^{e}. CAS Number 3032888-43-7
^{f}. CAS Number 83-46-5
^{g}. CAS Number 112-80-1
Asterisks (*) indicate chiral centers for ALC-0315.

LNP formulations (i) and (ii) may be prepared according to the procedure set forth in Example 2.

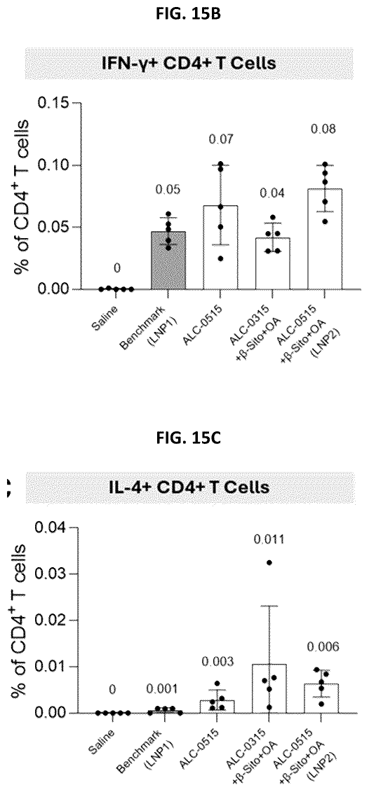

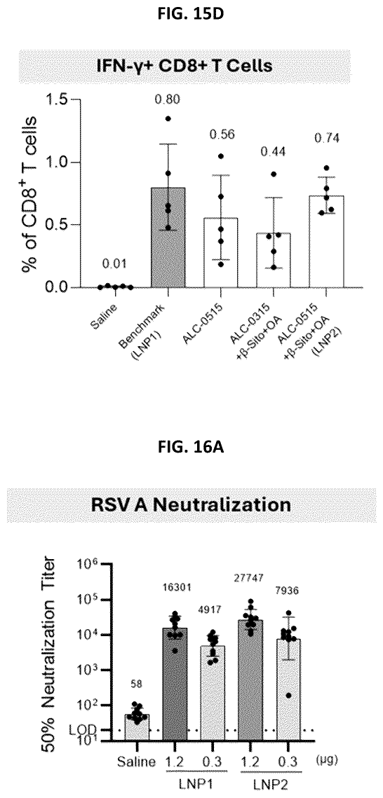

LNP formulations (iii) and (iv) may be prepared according to the procedures described in, for example Examples 16 and 17 and FIGS. 15A & 15B of International Application No. PCT/IB2025/052355 filed Mar. 4, 2025, which is hereby incorporated by reference herein in its entirety. Some exemplary methods include, but are not limited to:
(i) mixing one or more nucleic acids (aqueous phase) with the lipids of an LNP (organic phase), filtering the mixture via Tangential Flow Filtration/BBR, dissolving a fatty acid salt in a buffer or dissolving a fatty acid mixed with a compound in a buffer to make a fatty acid salt, in an amount that will achieve the desired O:R ratio of the composition, compounding the buffer solution with the filtered mixture, which then undergoes terminal filtration;
(ii) mixing one or more nucleic acids (aqueous phase) with an organic phase comprising the lipids of an LNP and a fatty acid in an amount that will achieve the desired O:R ratio of the composition, filtering the mixture via Tangential Flow Filtration/BBR, compounding the filtered mixture which then undergoes terminal filtration;
(iii) mixing one or more nucleic acids (aqueous phase) with the lipids of an LNP (organic phase), mixing a fatty acid salt or a fatty acid with a compound to make a fatty acid salt with a buffer in an amount that will achieve the desired O:R ratio of the composition (quench buffer), combining the two mixtures and filtering via Tangential Flow Filtration/BBR, compounding the filtered mixture which then undergoes terminal filtration; or
(iv) mixing one or more nucleic acids (aqueous phase) with an organic phase comprising the lipids of an LNP and a fatty acid salt in an amount that will achieve the desired O:R ratio of the composition, adding a quench buffer having pH 7.4 and filtering the mixture via Tangential Flow Filtration/BBR, compounding the filtered mixture which then undergoes terminal filtration.

RSV naïve female BALB/c mice were administrated two doses of 0.2 μg LNP-formulated modRNA as follows: female BALB/c mice (N=10 per group) were immunized IM with 0.2 μg of RSV A preF modRNA formulated in various LNP formulations described above. Mice were vaccinated on a two-dose schedule at Day 0 (DO) and Day 21 (D21) and serum was collected on Day 35 (D35/2 weeks post-dose 2). (FIG. 15A). On Day 35, all of the novel LNP formulations (LNP formulations ii-iv) demonstrated improved RSV neutralizing titers as compared to the LNP1 formulation. The LNP2 formulation containing both ALC-0515 and cholesterol:β-sitosterol with oleic acid mixture demonstrated a 10-fold improvement compared to the LNP1 formulation. Robust CD4+ Th-1 biased cell-mediated immunity was observed for both the LNP1 and LNP2 formulations, as indicated by elevated RSV-F-specific IFN-γ CD4+ compared to low RSV-F-specific IL-4 CD4+ T-cell responses (FIGS. 15B and 15C). RSV-F-specific IFN-γ CD8+ cell responses were also similar between the formulations (FIG. 15D). Overall, LNP2 demonstrated a significant increase in humoral response and comparable levels of the cellular immune response.

### Example 18—Combination of Hexavalent modRNAs Formulated in Two LNP Formulations

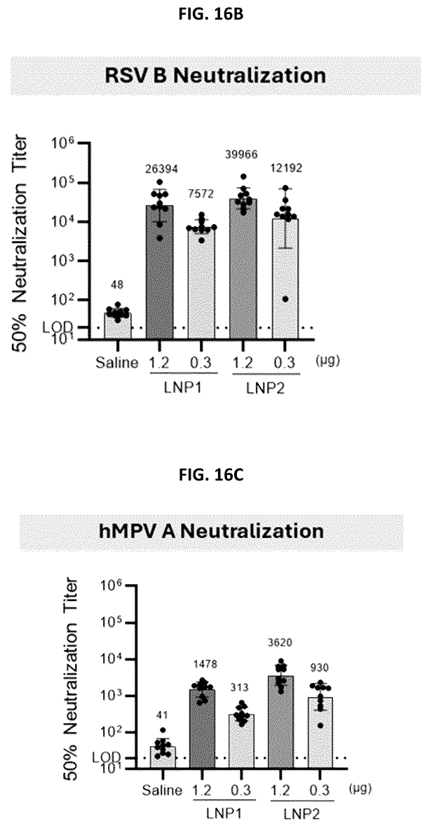

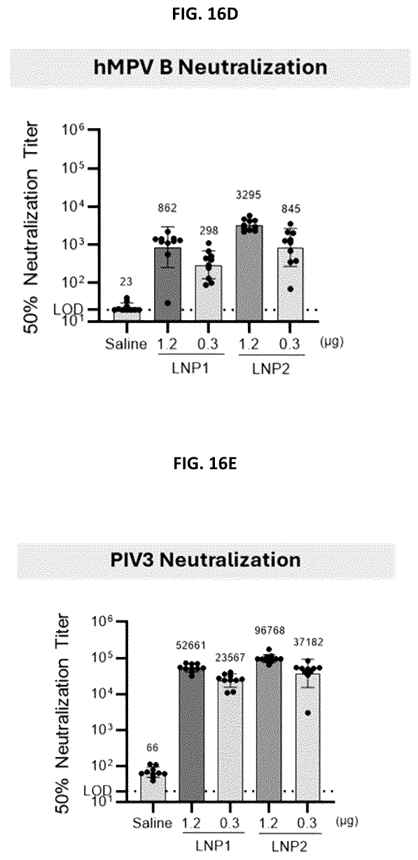

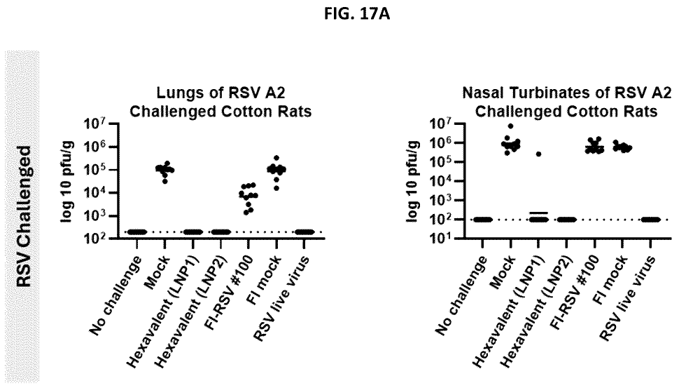

LNP2 was further evaluated in context of the hexavalent modRNAs, consisting of equal mRNA weight ratios (1:1:1:1:1:1) of co-formulated RSVpreF A, RSVpreF B, hMPVpreF A, hMPVpreF B, PIV3preF and PIV3HN. The benchmark LNP presentation, LNP1, was prepared in an identical manner for comparison. Naïve BALB/c mice received two doses of 1.2 μg or 0.3 μg of the hexavalent modRNAs formulated in either LNP1 or LNP2 at Days 0 and 21. On Day 35, both LNP formulations induced robust and comparable neutralizing responses against RSV A, RSV B, hMPV A, hMPV B, and PIV3 at both dose levels FIG. 16A-16E). LNP2-formulated groups trended higher compared with LNP1-formulated groups with close to 2-fold higher neutralizing responses across RSV A, RSV B, hMPV A, hMPV B, and PIV3. These results demonstrate that co-formulated hexavalent modRNA, LNP1 and LNP2 both induced robust neutralizing responses across RSV, hMPV and PIV3. LNP2 demonstrated more robust neutralizing responses compared to LNP1.

### Example 19—Demonstration of Efficacy and Absence of VAERD with modRNA RSV/hMPV/PIV3 in Various Formulation in the Cotton Rat Model

Cotton rats were immunized intramuscularly with saline (“Mock”), 45 μg RSV/hMPV/PIV3 modRNA (LNP1) vaccine, 45 μg RSV/hMPV/PIV3 modRNA (LNP2), 1:100 FI-RSV lot 100, or 1:100 FI-mock on Day 0 and 28. Control groups included cotton rats that received intranasal administration of live RSV (A2 strain), hMPV (TN/94-49 strain, A2 subgroup) or PIV3 (C243 strain) virus on Day 0. All groups were challenged on Day 49 with live RSV, hMPV or PIV3; lung and nasal turbinates were collected on 5 days following RSV and hMPV challenge and 4 days following PIV3 challenge.

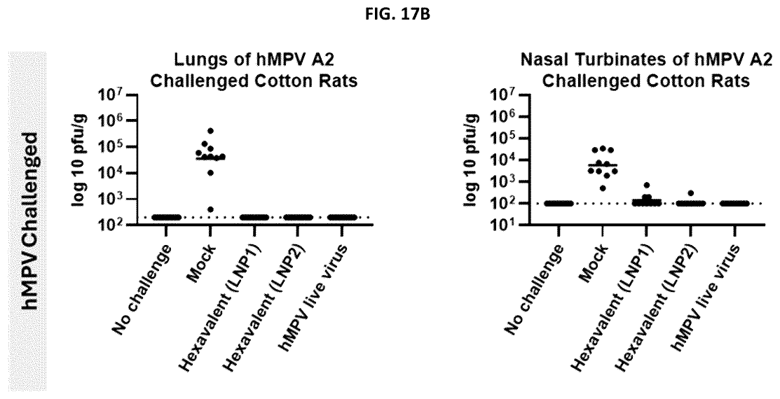

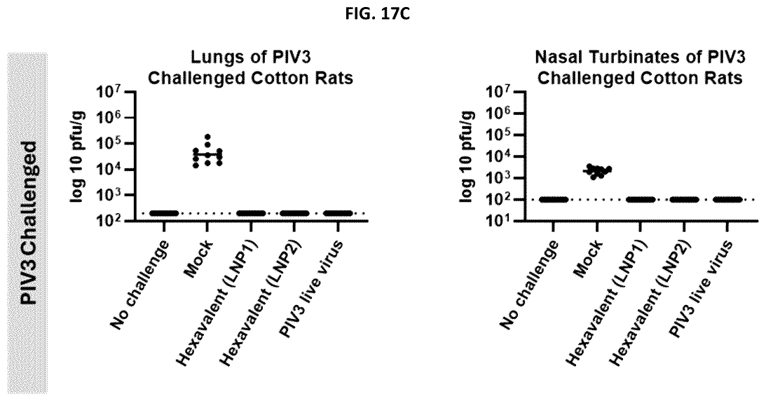

Viral loads from lungs and nasal turbinates were assessed five days after RSV and hMPV challenge or four days after PIV3 challenge. Immunization with the two LNP-modRNA vaccine candidates completely protected both the upper and lower respiratory tract from live RSV challenge (FIG. 17A-17C). The FI-RSV lot 100 group demonstrated partial protection of the lungs from RSV challenge and no protection in the nasal turbinates. In the hMPV challenge groups, both LNP-modRNA vaccine candidates also provided complete protection in the lower respiratory tract. No protection was observed in the Mock group that received saline control vaccination and challenged. In the PIV3 challenged groups, the LNP-modRNA vaccine candidates provided complete protection of the upper and lower respiratory tracts with baseline viral loads. All vaccine candidates provided complete or near-complete protection in both the lower and upper respiratory tract against RSV, hMPV and PIV3 challenge.

The neutralizing antibody titers are assessed by microneutralization assays against RSV A, RSV B, hMPV A, hMPV B, and PIV3 as described in Examples 3, 4 and 6, respectively.

### Lung and Nasal Turbinate Viral Titration

Individual lung and nasal turbinate homogenates were clarified by centrifugation and diluted 1:10 and 1:100 in EMEM. Confluent cell monolayers were infected in duplicates with 50 μL per well starting with undiluted (neat) samples followed by diluted homogenates in 24-well plates. After one hour incubation at 37° C. in a 5% CO₂ incubator, wells were overlaid with 0.75% methylcellulose medium, and plates were stored into a 37° C. 5% CO₂ incubator. After 4 days of incubation, the overlay was removed and the cells were fixed with 0.1% crystal violet stain for one hour, then rinsed, and air-dried. Plaques were counted and virus titers were expressed as plaque forming units (PFU) per gram of tissue. Viral titers in a group were calculated as the geometric mean±standard error for all animals in that group at a given time.

### Example 20—modRNA Respiratory Combo Suspension for Injection Drug Product Formulations

### 1. Product Description

The modRNA respiratory combination vaccine comprises drug products of modRNA bivalent Respiratory Syncytial Virus (RSV) PreF A & B, modRNA bivalent Human Metapneumovirus (hMPV) PreF A & B and/or modRNA bivalent Parainfluenza Virus (PIV3) PreF & HN and/or modRNA bivalent Parainfluenza Virus (PIV1) PreF & HN containing LNP1 or LNP2 lipid formulation compositions. Alternatively, the modRNA respiratory combination vaccine comprises drug products of modRNA bivalent Respiratory Syncytial Virus (RSV) PreF A & B, modRNA bivalent Human Metapneumovirus (hMPV) PreF A & B and/or modRNA Parainfluenza Virus (PIV3) PreF and/or modRNA Parainfluenza Virus (PIV1) PreF containing LNP1 or LNP2 lipid formulation compositions. In another embodiment, the modRNA respiratory combination vaccine comprises drug product of hexavalent or quadrivalent modRNA bivalent Respiratory Syncytial Virus (RSV) PreF A & B, modRNA bivalent Human Metapneumovirus (hMPV) PreF A & B and/or modRNA bivalent Parainfluenza Virus (PIV3) PreF & HN and/or modRNA bivalent Parainfluenza Virus (PIV1) PreF & HN containing LNP1 or LNP2 lipid formulation compositions.

Drug product (DP) formulations containing the description “LNP1” refers to drug product containing the following lipids: ALC-0315, ALC-0159, DSPC and cholesterol, which is described in Example 2.

Drug product formulations containing the description “LNP2” refers to drug product containing the following lipids: ALC-515, ALC-0159, DSPC, cholesterol, beta-sitosterol and oleic acid, which is described in Example 17.

The presentations of a frozen liquid drug product are listed below:
1. LNP1 DP suspension; containing bivalent RSV mRNA (1:1 mRNA weight ratio RSV preF A/B) Suspension for Injection (herein referred to as Bivalent RSV LNP1 DP),
2. LNP1 DP suspension; containing bivalent mRNA hMPV (1:1 mRNA weight ratio hMPV preF A/B) Suspension for Injection (herein referred to as Bivalent hMPV LNP1 DP),
3. LNP1 DP suspension; containing bivalent mRNA PIV3 (1:1 mRNA weight ratio PIV3 preF/HN), Suspension for Injection (herein referred to as Bivalent PIV3 LNP1 DP),
4. LNP1 DP suspension; containing bivalent mRNA PIV1 (1:1 mRNA weight ratio PIV1 preF/HN), Suspension for Injection (herein referred to as Bivalent PIV1 LNP1 DP),
5. LNP2 DP suspension; containing Bivalent RSV mRNA (1:1 mRNA weight ratio RSV preF A/B) Suspension for Injection (herein referred to as Bivalent RSV LNP2 DP),
6. LNP2 DP suspension; containing bivalent mRNA hMPV (1:1 mRNA weight ratio hMPV PreF A/B) Suspension for Injection (herein referred to as Bivalent hMPV LNP2 DP),
7. LNP2 DP suspension; containing bivalent mRNA PIV3 (1:1 mRNA weight ratio PIV3 preF/HN), Suspension for Injection (herein referred to as Bivalent PIV3 LNP2 DP), and/or
8. LNP2 DP suspension; containing bivalent mRNA PIV1 (1:1 mRNA weight ratio PIV1 preF/HN), Suspension for Injection (herein referred to as Bivalent PIV1 LNP2 DP).

The drug product is a preservative-free, sterile dispersion of lipid nanoparticles (LNPs) in aqueous cryoprotectant buffer for intramuscular administration. The vaccine drug product is formulated in 10 mM Tris buffer, 300 mM sucrose, pH 7.4.

The DP may be frozen (−80° C.). Alternatively, the DP comprising oleic acid or sodium oleate (e.g. LNP2) may be non-frozen/never frozen/liquid (NF) and stored at a refrigerated temperature (5° C.) or frozen then thawed (F/T) and stored at a refrigerated temperature (5° C.).

Filtered drug substance is stored at −15 to −25° C. The drug substance is stored frozen and is thawed, formulated into lipid nanoparticles, buffer exchanged via tangential flow filtration, filtered and then concentration is adjusted, and sucrose is added as a cryoprotectant to obtain the drug product.

### 2. Batch Formula

The batch formula for the LNP1 drug product consists of ALC-0315, ALC-0159, DSPC, cholesterol, sucrose, Tromethamine, Tris (hydroxymethyl) aminomethane hydrochloride and Water for Injection.

The batch formula for the LNP2 drug product consists of ALC-0515, ALC-0159, DSPC, cholesterol, beta-sitosterol, oleic acid, sucrose, Tromethamine, Tris (hydroxymethyl) aminomethane hydrochloride and Water for Injection.

The formulas for drug product suspensions are shown in Tables 67-74 for a Respiratory Combination Vaccine.

TABLE 67
Composition of Bivalent RSV LNP1 Drug
Product (Frozen Liquid Drug Product)

| Name of Ingredient | Function |
|---|---|
| Drug Substance: modRNA | Active ingredient |
| RSV PreF A | |
| Drug Substance: modRNA | Active ingredient |
| RSV PreF B | |
| ALC-0315^{a} | Functional lipid |
| ALC-0159^{b} | Functional lipid |
| DSPC^{c} | Structural lipid |
| Cholesterol | Structural lipid |
| Sucrose | Cryoprotectant |
| Tromethamine (Tris base) | Buffer component |
| Tris (hydroxymethyl) | Buffer component |
| aminomethane | |
| hydrochloride | |
| (Tris HCl) | |
| Water for Injection | Solvent |

^{a}ALC-0315 = ((4-hydroxybutyl)azanediyl)bis(hexane-6,1-diyl)bis(2-hexyldecanoate)
^{b}ALC-0159 = 2-[(polyethylene glycol)-2000]-N,N-ditetradecylacetamide
^{c}DSPC = 1,2-Distearoyl-sn-glycero-3-phosphocholine

TABLE 68
Composition of Bivalent hMPV LNP1 Drug
Product (Frozen Liquid Drug Product)

| Name of Ingredient | Function |
|---|---|
| Drug Substance: modRNA | Active ingredient |
| hMPV PreF A | |
| Drug Substance: modRNA | Active ingredient |
| hMPV PreF B | |
| ALC-0315^{a} | Functional lipid |
| ALC-0159^{b} | Functional lipid |
| DSPC^{c} | Structural lipid |
| Cholesterol | Structural lipid |
| Sucrose | Cryoprotectant |
| Tromethamine (Tris base) | Buffer component |
| Tris (hydroxymethyl) | Buffer component |
| aminomethane | |
| hydrochloride | |
| (Tris HCl) | |
| Water for Injection | Solvent |

^{a}ALC-0315 = ((4-hydroxybutyl)azanediyl)bis(hexane-6,1-diyl)bis(2-hexyldecanoate)
^{b}ALC-0159 = 2-[(polyethylene glycol)-2000]-N,N-ditetradecylacetamide
^{c}DSPC = 1,2-Distearoyl-sn-glycero-3-phosphocholine

TABLE 69
Composition of Bivalent PIV3 LNP1 Drug
Product (Frozen Liquid Drug Product)

| Name of Ingredient | Function |
|---|---|
| Drug Substance: modRNA | Active ingredient |
| PIV3 preF | |
| Drug Substance: modRNA | Active ingredient |
| PIV3 HN | |
| ALC-0315^{a} | Functional lipid |
| ALC-0159^{b} | Functional lipid |
| DSPC^{c} | Structural lipid |
| Cholesterol | Structural lipid |
| Sucrose | Cryoprotectant |
| Tromethamine (Tris base) | Buffer component |
| Tris (hydroxymethyl) | Buffer component |
| aminomethane | |
| hydrochloride | |
| (Tris HCl) | |
| Water for Injection | Solvent |

^{a}ALC-0315 = ((4-hydroxybutyl)azanediyl)bis(hexane-6,1-diyl)bis(2-hexyldecanoate)
^{b}ALC-0159 = 2-[(polyethylene glycol)-2000]-N,N-ditetradecylacetamide
^{c}DSPC = 1,2-Distearoyl-sn-glycero-3-phosphocholine

TABLE 70
Composition of Bivalent RSV LNP2 Drug
Product (Frozen Liquid Drug Product)

| Name of Ingredient | Function |
|---|---|
| Drug Substance: modRNA | Active ingredient |
| RSV PreF A | |
| Drug Substance: modRNA | Active ingredient |
| RSV PreF B | |
| ALC-0515^{a} | Functional lipid |
| ALC-0159^{b} | Functional lipid |
| DSPC^{c} | Structural lipid |
| Beta-sitosterol | Structural lipid |
| Cholesterol | Structural lipid |
| Oleic Acid | Structural lipid |
| Sucrose | Cryoprotectant |
| Tromethamine (Tris base) | Buffer component |
| Tris (hydroxymethyl) | Buffer component |
| aminomethane | |
| hydrochloride | |
| (Tris HCl) | |
| Water for Injection | Solvent |

^{a}ALC-0515 = bis(2-hexyldecyl) 6,6′-({2-[methyl(4-octanamidobutyl)amino]ethyl}azanediyl)dihexanoate
^{b}ALC-0159 = 2-[(polyethylene glycol)-2000]-N,N-ditetradecylacetamide
^{c}DSPC = 1,2-Distearoyl-sn-glycero-3-phosphocholine

TABLE 71
Composition of Bivalent hMPV LNP2 Drug
Product (Frozen Liquid Drug Product)

| Name of Ingredient | Function |
|---|---|
| Drug Substance: modRNA | Active ingredient |
| hMPV PreF A | |
| Drug Substance: modRNA | Active ingredient |
| hMPV PreF B | |
| ALC-0515^{a} | Functional lipid |
| ALC-0159^{b} | Functional lipid |
| DSPC^{c} | Structural lipid |
| beta-sitosterol | Structural lipid |
| Cholesterol | Structural lipid |
| Oleic Acid | Structural lipid |
| Sucrose | Cryoprotectant |
| Tromethamine (Tris base) | Buffer component |
| Tris (hydroxymethyl) | Buffer component |
| aminomethane | |
| hydrochloride | |
| (Tris HCl) | |
| Water for Injection | Solvent |

^{a}ALC-0515 = bis(2-hexyldecyl) 6,6′-({2-[methyl(4-octanamidobutyl)amino]ethyl}azanediyl)dihexanoate
^{b}ALC-0159 = 2-[(polyethylene glycol)-2000]-N,N-ditetradecylacetamide
^{c}DSPC = 1,2-Distearoyl-sn-glycero-3-phosphocholine

TABLE 72
Composition of Bivalent PIV3 LNP2 Drug
Product (Frozen Liquid Drug Product)

| Name of Ingredient | Function |
|---|---|
| Drug Substance: modRNA | Active ingredient |
| PIV3 preF | |
| Drug Substance: modRNA | Active ingredient |
| PIV3-HN | |
| ALC-0515^{a} | Functional lipid |
| ALC-0159^{b} | Functional lipid |
| DSPC^{c} | Structural lipid |
| beta-sitosterol | Structural lipid |
| Cholesterol | Structural lipid |
| Oleic Acid | Structural lipid |
| Sucrose | Cryoprotectant |
| Tromethamine (Tris base) | Buffer component |
| Tris (hydroxymethyl) | Buffer component |
| aminomethane | |
| hydrochloride | |
| (Tris HCl) | |
| Water for Injection | Solvent |

^{a}ALC-0515 = bis(2-hexyldecyl) 6,6′-({2-[methyl(4-octanamidobutyl)amino]ethyl}azanediyl)dihexanoate
^{b}ALC-0159 = 2-[(polyethylene glycol)-2000]-N,N-ditetradecylacetamide
^{c}DSPC = 1,2-Distearoyl-sn-glycero-3-phosphocholine

TABLE 73
Composition of Bivalent PIV1 LNP1 Drug
Product (Frozen Liquid Drug Product)

| Name of Ingredient | Function |
|---|---|
| Drug Substance: modRNA | Active ingredient |
| PIV1 preF | |
| Drug Substance: modRNA | Active ingredient |
| PIV1 HN | |
| ALC-0315^{a} | Functional lipid |
| ALC-0159^{b} | Functional lipid |
| DSPC^{c} | Structural lipid |
| Cholesterol | Structural lipid |
| Sucrose | Cryoprotectant |
| Tromethamine (Tris base) | Buffer component |
| Tris (hydroxymethyl) | Buffer component |
| aminomethane | |
| hydrochloride | |
| (Tris HCl) | |
| Water for Injection | Solvent |

^{a}ALC-0315 = ((4-hydroxybutyl)azanediyl)bis(hexane-6,1-diyl)bis(2-hexyldecanoate)
^{b}ALC-0159 = 2-[(polyethylene glycol)-2000]-N,N-ditetradecylacetamide
^{c}DSPC = 1,2-Distearoyl-sn-glycero-3-phosphocholine

TABLE 74
Composition of Bivalent PIV3 LNP2 Drug
Product (Frozen Liquid Drug Product)

| Name of Ingredient | Function |
|---|---|
| Drug Substance: modRNA | Active ingredient |
| PIV1 preF | |
| Drug Substance: modRNA | Active ingredient |
| PIV1-HN | |
| ALC-0515^{a} | Functional lipid |
| ALC-0159^{b} | Functional lipid |
| DSPC^{c} | Structural lipid |
| beta-sitosterol | Structural lipid |
| Cholesterol | Structural lipid |
| Oleic Acid | Structural lipid |
| Sucrose | Cryoprotectant |
| Tromethamine (Tris base) | Buffer component |
| Tris (hydroxymethyl) | Buffer component |
| aminomethane | |
| hydrochloride | |
| (Tris HCl) | |
| Water for Injection | Solvent |

^{a}ALC-0515 = bis(2-hexyldecyl) 6,6′-({2-[methyl(4-octanamidobutyl)amino]ethyl}azanediyl)dihexanoate
^{b}ALC-0159 = 2-[(polyethylene glycol)-2000]-N,N-ditetradecylacetamide
^{c}DSPC = 1,2-Distearoyl-sn-glycero-3-phosphocholine

### 3. Instructions for Preparing Dosing Solutions

Vials of each DP are transferred directly from −80° C. and allowed to thaw before administration. To obtain the 15 μg dose, 1.4 mL of 0.9% sodium chloride is added to 0.68 mL of 0.18 mg/mL bivalent DP to obtain a final dosing concentration of 0.06 mg/mL. Methods for preparation of additional doses (e.g. 30, 45, 60, 75, 90 and 100 μg) are known by one of skill in the art.

### 4. Excipients

Excipients present in the DPs are shown in Tables 67-72 hereinabove. The choice of buffer and excipients is based on early phase formulation development work. Tromethamine (Tris base) and Tris Hydrochloride (Tris HCl) are buffer components used in pharmaceuticals and suitable to achieve the desired product pH. Sucrose was selected for its stabilizing effect to enable storage in the frozen state.

The lipid excipients in the drug products are further described in Table 73.

TABLE 75
Lipid Excipients in the Drug Product

| | | | Physical | |
|---|---|---|---|---|
| | Molecular | | State and | |
| | Weight | Molecular | Storage | Chemical Name (Synonyms) and |
| Lipid | [Da] | Formula | Condition | Structure |
| ALC-0315^{a} | 766 | C48H95NO5 | Liquid (oil) | ((4-hydroxybutyl)azanediyl)bis(hexane-6,1-diyl)bis(2- hexyldecanoate) 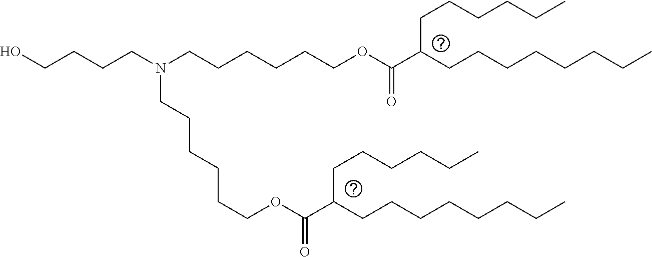 [SMILES: CCCCCCCCC(CCCCCC)C(=O)OCCCCCCN(CCCCO)CCCCCCOC(=O)C(CCCCCC)CCCCCCCC] |
| ALC-0159^{b} | 2100- 2700 | (C2H4O)n C31H63NO2 | Solid −20° C. | 2-[(polyethylene glycol)-2000]-N,N- ditetradecylacetamide 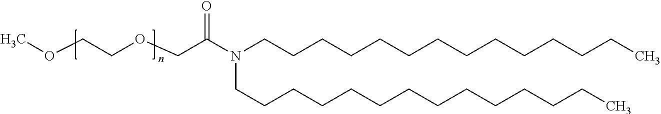 |
| DSPC^{c} | 790 | C44H88NO8 P | Solid −20° C. | 1,2-distearoyl-sn-glycero-3-phosphocholine 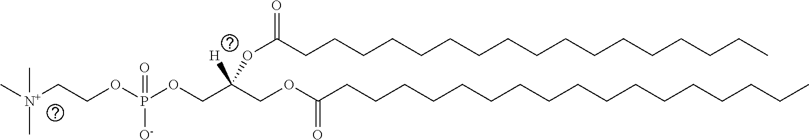 [SMILES: CCCCCCCCCCCCCCCCCC(=O)OC[C@H](COP(=O)([O-])OCC[N+](C)(C)C)OC(=O)CCCCCCCCCCCCCCCCC] |
| Cholesterol^{d} | 387 | C27H46O | Solid −20° C. | 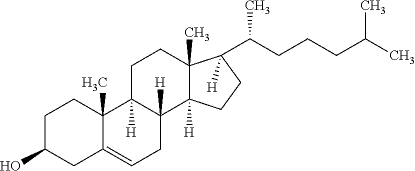 |
| ALC-0515^{e} | 948.6 | C59H117N3 O5 | Liquid (oil) | bis(2-hexyldecyl) 6,6′-({2-[methyl(4- octanamidobutyl)amino]ethyl}azanediyl)dihexanoate 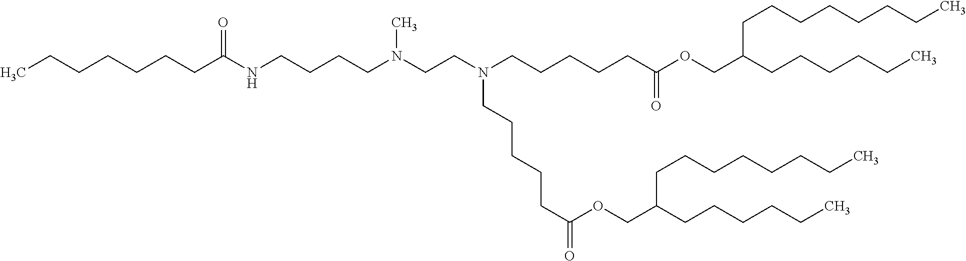 [SMILES: CCCCCCCCC(CCCCCC)COC(=O)CCCCCN(CCCCCC(=O)OCC(CCCCCC)CCCCCCCC)CCN(C)CCCCNC(=O)CCCCCCC] |
| beta- Sitosterol^{f} | 414.7 | C29H50O | Solid | (−)-beta-Sitosterol; 22,23-Dihydrostigmasterol 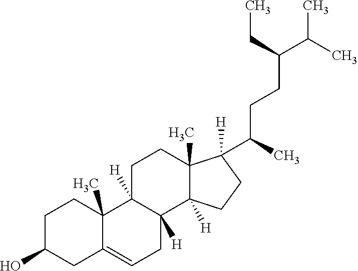 [SMILES: CC[C@H](CC[C@@H](C)[C@H]1CC[C@H]2[C@@H]3CC=C4C[C@@H](O)CC[C@]4(C)[C@H]3CC[C@]12C)C(C)C] |
| Oleic acid^{g} | 282.47 | C18H34O2 | Liquid (Oil) | (9Z)-octadec-9-enoic acid; (Z)-9-Octadecenoic acid 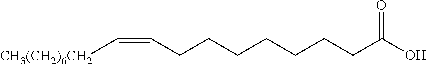 |

^{a}. CAS Number 2036272-55-4
^{b}. CAS Number 1849616-42-7
^{c}. CAS Number 816-94-4
^{d}. CAS Number 57-88-5
^{e}. CAS Number 3032888-43-7
^{f}. CAS Number 83-46-5
^{g}. CAS Number 112-80-1
Asterisks (*) indicate chiral centers for ALC-0315.

All of the methods disclosed and claimed herein can be made and executed without undue experimentation in light of the present disclosure. While the compositions and methods of this disclosure have been described in terms of certain aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

The contents of all cited references (including literature references, issued patents, published patent applications, and GENBANK® Accession numbers as cited throughout this application) recited in the application, to the extent that they provide exemplary procedural or other details supplementary to those set forth herein, are hereby specifically and expressly incorporated by reference. When definitions of terms in documents that are incorporated by reference herein conflict with those used herein, the definitions used herein govern.

1. An RNA molecule comprising at least one open reading frame encoding a human Metapneumovirus (hMPV) fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.

2. The RNA molecule of claim 1, wherein the hMPV F polypeptide comprises at least one mutation relative to the wild-type hMPV F polypeptide, wherein the mutation is selected from:
(1) Q100R, S101R, and D185P;
(2) Q100R, S101R, L110C, T127C, A140C, A147C, N153C, D185P, L219K, V231I, N322C, T365C, E453Q, and V463C;
(3) V84C, A140C, A147C, D185P, A249C, D454C, and V458C, and having deletions of amino acids at positions 89-112 replaced with a GSGGSG linker beginning at position 89;
(4) Q100R, S101R, L110C, T127C, A140C, A147C, N153C, A185P, L219K, V231I, N322C, T365C, E453Q, and V463C; or
(5) V84C, A140C, A147C, A185P, A249C, D454C, and V458C, and having deletions of amino acids at positions 89-112 replaced with a GSGGSG linker beginning at position 89, wherein the amino acid positions correspond to the amino acid sequence set forth in SEQ ID NO: 639.

3.-4. (canceled)

5. An RNA molecule comprising at least one open reading frame encoding a parainfluenza virus type 3 (PIV3) fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.

6. The RNA molecule of claim 5, wherein the PIV3 F polypeptide comprises at least one mutation relative to the wild-type PIV3 F polypeptide, wherein the mutation is selected from:
(1) E209C and L234C;
(2) S160C, V170C, E209C, L234C, A463L, and S470L; or
(3) Q162C, L168C, I213C, G230C, A463V, and I474Y,
wherein the amino acid positions correspond to the amino acid sequence set forth in SEQ ID NO: 690.

7.-8. (canceled)

9. An RNA molecule comprising at least one open reading frame encoding a parainfluenza virus type 3 (PIV3) hemagglutinin-neuraminidase protein (HN) polypeptide.

10. The RNA molecule of claim 9, wherein the PIV3 HN polypeptide comprises at least one mutation relative to the wild-type PIV3 HN polypeptide, wherein the mutation is selected from:
i) a deletion of residues at positions 59-88; or
ii) a deletion of residues at positions 59-130,
wherein the amino acid positions correspond to the amino acid sequence set forth in SEQ ID NO: 724.

11.-13. (canceled)

14. An RNA molecule comprising at least one open reading frame encoding a parainfluenza virus type 1 (PIV1) fusion protein F (F) polypeptide and a 5′ untranslated region (5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.

15. The RNA molecule of claim 14, wherein the PIV1 F polypeptide comprises at least one mutation relative to the wild-type PIV1 F polypeptide, wherein the mutation is selected from:
(1) Q92C, F113G, F114S, G134C, A466L, S473L, and A480L; or
(2) F113G, F114S, G134A, A466L, and S473L,
wherein the amino acid positions correspond to the amino acid sequence set forth in SEQ ID NO: 672.

16.-18. (canceled)

19. An RNA molecule comprising at least one open reading frame encoding a parainfluenza virus type 1 (PIV1) hemagglutinin-neuraminidase protein (HN) polypeptide.

20. The RNA molecule of claim 19, wherein the PIV1 HN polypeptide comprises at least one mutation relative to the wild-type PIV1 HN polypeptide, wherein the mutation is selected from:
i) a deletion of residues at positions 57-84; or
ii) a deletion of residues at positions 57-129,
wherein the amino acid positions correspond to the amino acid sequence set forth in SEQ ID NO: 750.

21.-31. (canceled)

32. An immunogenic composition comprising at least one RNA molecule of claim 1, wherein the RNA molecule is formulated in a lipid nanoparticle (LNP) thereby forming an RNA-LNP, and optionally wherein the immunogenic composition further comprises an RNA molecule comprising at least one open reading frame encoding a respiratory syncytial virus (RSV) fusion protein F (F) polypeptide and a 5′ untranslated region (RSV 5′ UTR), wherein the 5′ UTR comprises a nucleic acid sequence at least 90% identical to a nucleic acid sequence selected from the group consisting of SEQ ID NOs: 336-344, 356-358, 360, 362-367, 370-375, and 378-396.

33. (canceled)

34. The RNA molecule of claim 32, wherein the RSV F polypeptide comprises at least one mutation relative to the wild-type RSV F polypeptide, wherein the mutation is selected from:
(1) A103C, I148C, S190I and D486S;
(2) T54H, A103C, I148C, S190I, V296I and D486S; or
(3) T54H S55C, L188C and D486S,
wherein the amino acid positions correspond to the amino acid sequence set forth in SEQ ID NO: 1.

35.-42. (canceled)

43. The immunogenic composition of claim 32, wherein the immunogenic composition comprises one or more RNA-LNPs selected from:
i. an RNA-LNP comprising one RNA molecule,
ii. an RNA-LNP comprising two or more co-formulated RNA molecules that do not encode the same antigen (pre-mixed), or
iii. a mixture of two or more RNA-LNPs selected from (i) or (ii) (post-mix).

44. The immunogenic composition of claim 43 comprising:
i) a first and a second RNA molecule;
ii) a first, a second and a third RNA molecule;
iii) a first, a second, a third and a fourth RNA molecule;
iv) a first, a second, a third, a fourth, and a fifth RNA molecule;
v) a first, a second, a third, a fourth, a fifth, and a sixth RNA molecule;
vi) a first, a second, a third, a fourth, a fifth, a sixth, and a seventh RNA molecule; or
vii) a first, a second, a third, a fourth, a fifth, a sixth, a seventh, and an eighth RNA molecule,
wherein the first, second, third, fourth, fifth, sixth, seventh and eighth RNA molecules of the immunogenic composition do not encode the same antigen and are formulated in one LNP or in separate LNPs, and further wherein
i. the first antigen is an hMPV preF A polypeptide;
ii. the second antigen is an hMPV preF B polypeptide;
iii. the third antigen is an RSV preF A polypeptide;
iv. the fourth antigen is an RSV preF B polypeptide;
V. the fifth antigen is a PIV3 preF polypeptide;
vi. the sixth antigen is a PIV3 HN polypeptide;
vii. the seventh antigen is a PIV1 F polypeptide; and
viii. the eighth antigen is a PIV1 HN polypeptide.

45. The immunogenic composition of claim 32, wherein lipid nanoparticle comprises:
(i) at least one of a steroid or steroid analog or a mixture of a steroid analog and a steroid,
(ii) a neutral lipid,
(iii) a PEGylated lipid, and
(iv) a cationic lipid.

46.-47. (canceled)

48. The immunogenic composition of claim 45, wherein the mixture of a steroid analog and a steroid comprises a mixture of beta-sitosterol and cholesterol having a molar ratio of beta-sitosterol:cholesterol of 6:4.

49. (canceled)

50. The immunogenic composition of claim 32, wherein
the immunogenic composition further comprises a fatty acid, a derivative or salt thereof.

51. (canceled)

52. The immunogenic composition of claim 50, wherein the immunogenic composition has a fatty acid or salt thereof to RNA weight ratio (O:R) of at least 1.5:1.

53. The immunogenic composition of claim 52, wherein the immunogenic composition has an O:R selected from about 1.5:1, about 2:1, about 3:1, about 4:1, about 6:1, about 6.5:1, about 8:1, about 10:1, about 12:1, about 13:1 or about 24:1.

54.-60. (canceled)

61. The immunogenic composition of claim 44, wherein the immunogenic composition is administered in an effective amount to induce an immune response in a subject administered at least one dose of the vaccine.

62. The immunogenic composition of claim 61, wherein the efficacy of the vaccine in vaccinated subjects is at least 60%, relative to unvaccinated subjects, following a single dose of the vaccine.

63. The immunogenic composition of claim 44, wherein the effective amount is sufficient to produce protective levels of neutralizing antibody against the antigenic hMPV F protein and/or the antigenic PIV3 F protein and/or the antigenic PIV3 HN protein and/or the antigenic RSV F protein and/or the antigenic PIV1 F protein and/or the antigenic PIV1 HN protein as measured in serum of a subject vaccinated with at least one dose of the vaccine.

64. (canceled)

65. A mutant of a wild-type parainfluenza virus type 1 (PIV1) HN polypeptide, wherein the mutant comprises at least one amino acid mutation relative to the amino acid sequence of the wild type PIV1 HN polypeptide, wherein the mutation is selected from the group consisting of: i) a deletion of residues at positions 57-84; and ii) a deletion of residues at positions 57-129, wherein the amino acid positions are numbered according to SEQ ID NO: 750.

66. A mutant of a wild-type parainfluenza virus type 3 (PIV3) HN polypeptide, wherein the mutant comprises at least one amino acid mutation relative to the amino acid sequence of the wild type PIV3 HN polypeptide, wherein the mutation is selected from the group consisting of: i) a deletion of residues at positions 59-88; and ii) a deletion of residues at positions 59-130, wherein the amino acid positions are numbered according to SEQ ID NO: 724.

67. A method of inducing an immune response against RSV, hMPV and/or PIV3 and/or PIV1 in a subject, comprising administering to the subject an effective amount of the immunogenic composition of claim 44.

68. A method of preventing, treating or ameliorating an infection, disease or condition associated with RSV, hMPV and/or PIV3 and/or PIV1 in a subject, comprising administering to a subject an effective amount of the immunogenic composition of claim 44.

69.-71. (canceled)
